SEQUENCE LISTING (1059 sequences)

SEQ ID NO: 1 - protein, 10 aa, synthetic construct
  REGION 1..10: note = HLA-E CDR-L1
SASSSVSYMH

SEQ ID NO: 2 - protein, 7 aa, synthetic construct
  REGION 1..7: note = HLA-E CDR-L2
GTSNLAS

SEQ ID NO: 3 - protein, 9 aa, synthetic construct
  REGION 1..9: note = HLA-E CDR-L3
QQRSRYPFT

SEQ ID NO: 4 - protein, 7 aa, synthetic construct
  REGION 1..7: note = HLA-E CDR-H1
GFSLTSY

SEQ ID NO: 5 - protein, 5 aa, synthetic construct
  REGION 1..5: note = HLA-E CDR-H2
WTGGT

SEQ ID NO: 6 - protein, 13 aa, synthetic construct
  REGION 1..13: note = HLA-E CDR-H3
DGDSYNREAWFAY

SEQ ID NO: 7 - protein, 106 aa, synthetic construct
  REGION 1..106: note = HLA-E light chain
QIVLTQSPAVISASPGEKVILTCSASSSVSYMHWFQQKPGTSPKLWIYGTSNLASGVPARFSGGGSGTSYSLTISRMEAEDAATYYCQQRSRYPFTFGSGTKLEIK

SEQ ID NO: 8 - protein, 121 aa, synthetic construct
  REGION 1..121: note = HLE-E heavy chain
QVQLRESGPSLVKPSQTLSLTCTVSGFSLTSYGVHWVRQPPGKGLEWLGVIWTGGTTNYNTALMSRLSITKDNSKSQVFLKMNSLQTDDTAIYYCARDGDSYNREAWFAYWGQGTLVTVSA

SEQ ID NO: 9 - skipped

SEQ ID NO: 10 - protein, 7 aa, synthetic construct
  REGION 1..7: note = HLA-A11 heavy chain CDR1
SGGYYWS

SEQ ID NO: 11 - protein, 7 aa, synthetic construct
  REGION 1..7: note = HLA-A11 heavy chain CDR1
TSGVGVG

SEQ ID NO: 12 - protein, 5 aa, synthetic construct
  REGION 1..5: note = HLA-A11 heavy chain CDR1
SYAMH

SEQ ID NO: 13 - protein, 5 aa, synthetic construct
  REGION 1..5: note = HLA-A11 heavy chain CDR1
SYDMH

SEQ ID NO: 14 - protein, 5 aa, synthetic construct
  REGION 1..5: note = HLA-A11 heavy chain CDR1
SYWMH

SEQ ID NO: 15 - protein, 16 aa, synthetic construct
  REGION 1..16: note = HLA-A11 heavy chain CDR2
YIYYSGSTYYNPSLKS

SEQ ID NO: 16 - protein, 16 aa, synthetic construct
  REGION 1..16: note = HLA-A11 heavy chain CDR2
LIYWNDDKRYSPSLKS

SEQ ID NO: 17 - protein, 17 aa, synthetic construct
  REGION 1..17: note = HLA-A11 heavy chain CDR2
WINAGNGNTKYSQKFQG

SEQ ID NO: 18 - protein, 16 aa, synthetic construct
  REGION 1..16: note = HLA-A11 heavy chain CDR2
AIGTAGDTYYPGSVKG

SEQ ID NO: 19 - protein, 17 aa, synthetic construct
  REGION 1..17: note = HLA-A11 heavy chain CDR2
RINSDGSSTSYADSVKG

SEQ ID NO: 20 - protein, 9 aa, synthetic construct
  REGION 1..9: note = HLA-A11 heavy chain CDR3
HYYYYSMDV

SEQ ID NO: 21 - protein, 9 aa, synthetic construct
  REGION 1..9: note = HLA-A11 heavy chain CDR3
HYYYYYLDV

SEQ ID NO: 22 - protein, 8 aa, synthetic construct
  REGION 1..8: note = HLA-A11 heavy chain CDR3
HYYYYMDV

SEQ ID NO: 23 - protein, 9 aa, synthetic construct
  REGION 1..9: note = HLA-A11 heavy chain CDR3
HYYYYYMDV

SEQ ID NO: 24 - protein, 9 aa, synthetic construct
  REGION 1..9: note = HLA-A11 heavy chain CDR3
KTTSFYFDY

SEQ ID NO: 25 - protein, 10 aa, synthetic construct
  REGION 1..10: note = HLA-A11 heavy chain CDR3
RHMRLSCFDY

SEQ ID NO: 26 - protein, 12 aa, synthetic construct
  REGION 1..12: note = HLA-A11 heavy chain CDR3
EGNGANPDAFDI

SEQ ID NO: 27 - protein, 11 aa, synthetic construct
  REGION 1..11: note = HLA-A11 heavy chain CDR3
DLPGSYWYFDL

SEQ ID NO: 28 - protein, 10 aa, synthetic construct
  REGION 1..10: note = HLA-A11 heavy chain CDR3
GVLLYNWFDP

SEQ ID NO: 29 - protein, 11 aa, synthetic construct
  REGION 1..11: note = HLA-A11 light chain CDR1
RASQSISSYLN

SEQ ID NO: 30 - protein, 7 aa, synthetic construct
  REGION 1..7: note = HLA-A11 light chain CDR2
AASSLQS

SEQ ID NO: 31 - protein, 9 aa, synthetic construct
  REGION 1..9: note = HLA-A11 light chain CDR3
QQSYSTPLT

SEQ ID NO: 32 - protein, 119 aa, synthetic construct
  REGION 1..119: note = HLA-A11 heavy chain
QVQLQESGPGLVKPSQTLSLTCTVSGGSISSGGYYWSWIRQPPGKGLEWIGYIYYSGSTYYNPSLKSRVTISVDTSKNQFSLKLSSVTAADTAVYYCARHYYYYSMDVWGKGTTVTVSS

SEQ ID NO: 33 - protein, 120 aa, synthetic construct
  REGION 1..120: note = HLA-A11 heavy chain
QITLKESGPTLVKPTQTLTLTCTFSGFSLSTSGVGVGWIRQPPGKALEWLALIYWNDDKRYSPSLKSRLTITKDTSKNQVVLTMTNMDPVDTATYYCAHRHMRLSCFDYWGQGTLVTVSS

SEQ ID NO: 34 - protein, 121 aa, synthetic construct
  REGION 1..121: note = HLA-A11 heavy chain
QVQLVQSGAEVKKPGASVKVSCKASGYTFTSYAMHWVRQAPGQRLEWMGWINAGNGNTKYSQKFQGRVTITRDTSASTAYMELSSLRSEDTAVYYCAREGNGANPDAFDIWGQGTMVTVSS

SEQ ID NO: 35 - protein, 119 aa, synthetic construct
  REGION 1..119: note = HLA-A11 heavy chain
EVQLVESGGGLVQPGGSLRLSCAASGFTFSSYDMHWVRQATGKGLEWVSAIGTAGDTYYPGSVKGRFTISRENAKNSLYLQMNSLRAGDTAVYYCARDLPGSYWYFDLWGRGTLVTVSS

SEQ ID NO: 36 - protein, 119 aa, synthetic construct
  REGION 1..119: note = HLA-A11 heavy chain
QVQLQESGPGLVKPSQTLSLTCTVSGGSISSGGYYWSWIRQPPGKGLEWIGYIYYSGSTYYNPSLKSRVTISVDTSKNQFSLKLSSVTAADTAVYYCARHYYYYYLDVWGKGTTVTVSS

SEQ ID NO: 37 - protein, 119 aa, synthetic construct
  REGION 1..119: note = HLA-A11 heavy chain
EVQLVESGGGLVQPGGSLRLSCAASGFTFSSYWMHWVRQAPGKGLVWVSRINSDGSSTSYADSVKGRFTISRDNAKNTLYLQMNSLRAEDTAVYYCCLGVLLYNWFDPWGQGTLVTVSS

SEQ ID NO: 38 - protein, 118 aa, synthetic construct
  REGION 1..118: note = HLA-A11 heavy chain
QVQLQESGPGLVKPSQTLSLTCTVSGGSISSGGYYWSWIRQPPGKGLEWIGYIYYSGSTYYNPSLKSRVTISVDTSKNQFSLKLSSVTAADTAVYYCARHYYYYMDVWGKGTTVTVSS

SEQ ID NO: 39 - protein, 119 aa, synthetic construct
  REGION 1..119: note = HLA-A11 heavy chain
QITLKESGPTLVKPTQTLTLTCTFSGFSLSTSGVGVGWIRQPPGKALEWLALIYWNDDKRYSPSLKSRLTITKDTSKNQVVLTMTNMDPVDTATYYCAHKTTSFYFDYWGQGTLVTVSS

SEQ ID NO: 40 - protein, 119 aa, synthetic construct
  REGION 1..119: note = HLA-A11 heavy chain
QVQLQESGPGLVKPSQTLSLTCTVSGGSISSGGYYWSWIRQPPGKGLEWIGYIYYSGSTYYNPSLKSRVTISVDTSKNQFSLKLSSVTAADTAVYYCARHYYYYYMDVWGKGTTVTVSS

SEQ ID NO: 41 - protein, 107 aa, synthetic construct [sequence omitted]

SEQ ID NO: 42 - DNA, 357 bp, synthetic construct
  misc_feature 1..357: note = HLA-A11 heavy chain
  source 1..357: mol_type = other DNA
caggtgcagctgcaggaaagcggccctggcctggtgaaacccagccagaccctgagcctgacctgcacagtgtccggcggctcgatcagcagcggcggctactactggtcctggatcagacagccccctggcaagggcctggaatggatcggctacatctactacagcggcagcacctactacaaccccagcctgaagtccagagtgaccatcagcgtggacaccagcaagaaccagttcagcctgaagctgagcagcgtgacagccgccgacaccgctgtgtattactgtgcgagacactactactactactccatggacgtctggggcaaagggaccacggtcaccgtgtcctca

SEQ ID NO: 43 - DNA, 360 bp, synthetic construct
  misc_feature 1..360: note = HLA-A11 heavy chain
  source 1..360: mol_type = other DNA
cagatcaccctgaaagagtccggccccaccctggtgaaacccacccagaccctgaccctgacatgcaccttcagcggcttcagcctgagcacctctggcgtgggcgtgggctggatcagacagcctcccggcaaggccctggaatggctggccctgatctactggaacgacgacaagcggtacagccccagcctgaagtcccggctgaccatcaccaaggacacctcgaagaaccaggtggtgctgaccatgacaaacatggaccccgtggacaccgccacatattactgtgcacacagacacatgcgtttaagctgttttgactactggggccagggaaccctggtcaccgtgtcctca

SEQ ID NO: 44 - DNA, 363 bp, synthetic construct
  misc_feature 1..363: note = HLA-A11 heavy chain
  source 1..363: mol_type = other DNA
caggtgcagctggtgcagtctggcgccgaagtgaagaaacctggcgcctccgtgaaggtgtcctgcaaggccagcggctacaccttcaccagctacgccatgcactgggttcgacaggcccctggccagagactggaatggatgggctggatcaacgccggcaacggcaacaccaagtacagccagaaattccagggcagagtgaccatcacccgggacaccagcgccagcaccgcctacatggaactgagcagcctgcggagcgaggacaccgctgtgtattactgtgcgagagaaggaaatggtgccaaccctgatgcttttgatatctggggccaagggacaatggtcaccgtgtcctca

SEQ ID NO: 45 - DNA, 357 bp, synthetic construct
  misc_feature 1..357: note = HLA-A11 heavy chain
  source 1..357: mol_type = other DNA
gaagtgcagctggtggaaagcggcggaggcctggtgcagcctggcggcagcctgagactgtcttgcgccgccagcggcttcaccttcagcagctacgacatgcactgggtccgccaggccaccggcaagggactggaatgggtgtccgccatcggcacagccggcgacacttactaccccggcagcgtgaagggccggttcaccatcagcagagagaacgccaagaacagcctgtacctgcagatgaacagccttcgagccggcgataccgccgtgtattactgtgcaagagatctccctggtagctactggtacttcgatctctggggccgtggcaccctggtcactgtgtcctca

SEQ ID NO: 46 - DNA, 357 bp, synthetic construct
  misc_feature 1..357: note = HLA-A11 heavy chain
  source 1..357: mol_type = other DNA
caggtgcagctgcaggaaagcggccctggcctggtgaaacccagccagaccctgagcctgacctgcacagtgtccggcggctcgatcagcagcggcggctactactggtcctggatcagacagccccctggcaagggcctggaatggatcggctacatctactacagcggcagcacctactacaaccccagcctgaagtccagagtgaccatcagcgtggacaccagcaagaaccagttcagcctgaagctgagcagcgtgacagccgccgacaccgctgtgtattactgtgcgagacactactactactactacctggacgtctggggcaaagggaccacggtcaccgtgtcctca

SEQ ID NO: 47 - DNA, 357 bp, synthetic construct
  misc_feature 1..357: note = HLA-A11 heavy chain
  source 1..357: mol_type = other DNA
gaagtgcagctggtggaaagcggcggaggcctggtgcagcctggcggcagcctgagactgtcttgcgccgccagcggcttcaccttcagcagctactggatgcactgggtccgccaggcccctggcaagggactggtctgggtgtctcgaatcaacagcgacggcagcagcaccagctacgccgacagcgtgaagggccggttcaccatcagccgggacaacgccaagaacaccctgtacctgcagatgaacagcctgcgggccgaggacaccgccgtgtattactgttgtttgggtgttttattatacaactggttcgacccctggggccagggaaccctggtcaccgtgtcctca

SEQ ID NO: 48 - DNA, 354 bp, synthetic construct
  misc_feature 1..354: note = HLA-A11 heavy chain
  source 1..354: mol_type = other DNA
caggtgcagctgcaggaaagcggccctggcctggtgaaacccagccagaccctgagcctgacctgcacagtgtccggcggctcgatcagcagcggcggctactactggtcctggatcagacagccccctggcaagggcctggaatggatcggctacatctactacagcggcagcacctactacaaccccagcctgaagtccagagtgaccatcagcgtggacaccagcaagaaccagttcagcctgaagctgagcagcgtgacagccgccgacaccgctgtgtattactgtgcgagacactactactactacatggacgtctggggcaaagggaccacggtcaccgtgtcctca

SEQ ID NO: 49 - DNA, 357 bp, synthetic construct
  misc_feature 1..357: note = HLA-A11 heavy chain
  source 1..357: mol_type = other DNA
cagatcaccctgaaagagtccggccccaccctggtgaaacccacccagaccctgaccctgacatgcaccttcagcggcttcagcctgagcacctctggcgtgggcgtgggctggatcagacagcctcccggcaaggccctggaatggctggccctgatctactggaacgacgacaagcggtacagccccagcctgaagtcccggctgaccatcaccaaggacacctcgaagaaccaggtggtgctgaccatgacaaacatggaccccgtggacaccgccacatattactgtgcacacaaaacgacgtcgttttactttgactactggggccagggaaccctggtcaccgtgtcctca

SEQ ID NO: 50 - DNA, 357 bp, synthetic construct
  misc_feature 1..357: note = HLA-A11 heavy chain
  source 1..357: mol_type = other DNA
caggtgcagctgcaggaaagcggccctggcctggtgaaacccagccagaccctgagcctgacctgcacagtgtccggcggctcgatcagcagcggcggctactactggtcctggatcagacagccccctggcaagggcctggaatggatcggctacatctactacagcggcagcacctactacaaccccagcctgaagtccagagtgaccatcagcgtggacaccagcaagaaccagttcagcctgaagctgagcagcgtgacagccgccgacaccgctgtgtattactgtgcgagacactactactactactacatggacgtctggggcaaagggaccacggtcaccgtgtcctca

SEQ ID NO: 51 - DNA, 321 bp, synthetic construct
  misc_feature 1..321: note = HLA-A11 light chain
  source 1..321: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctcactttcggcggcggaacaaaggtggagatcaag

SEQ ID NO: 52 - protein, 243 aa, synthetic construct
  REGION 1..243: note = HLA-A11 antigen binding domain
QVQLQESGPGLVKPSQTLSLTCTVSGGSISSGGYYWSWIRQPPGKGLEWIGYIYYSGSTYYNPSLKSRVTISVDTSKNQFSLKLSSVTAADTAVYYCARHYYYYSMDVWGKGTTVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 53 - protein, 244 aa, synthetic construct
  REGION 1..244: note = HLA-A11 antigen binding domain
QITLKESGPTLVKPTQTLTLTCTFSGFSLSTSGVGVGWIRQPPGKALEWLALIYWNDDKRYSPSLKSRLTITKDTSKNQVVLTMTNMDPVDTATYYCAHRHMRLSCFDYWGQGTLVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 54 - protein, 245 aa, synthetic construct
  REGION 1..245: note = HLA-A11 antigen binding domain
QVQLVQSGAEVKKPGASVKVSCKASGYTFTSYAMHWVRQAPGQRLEWMGWINAGNGNTKYSQKFQGRVTITRDTSASTAYMELSSLRSEDTAVYYCAREGNGANPDAFDIWGQGTMVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 55 - protein, 243 aa, synthetic construct
  REGION 1..243: note = HLA-A11 antigen binding domain
EVQLVESGGGLVQPGGSLRLSCAASGFTFSSYDMHWVRQATGKGLEWVSAIGTAGDTYYPGSVKGRFTISRENAKNSLYLQMNSLRAGDTAVYYCARDLPGSYWYFDLWGRGTLVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 56 - protein, 243 aa, synthetic construct
  REGION 1..243: note = HLA-A11 antigen binding domain
QVQLQESGPGLVKPSQTLSLTCTVSGGSISSGGYYWSWIRQPPGKGLEWIGYIYYSGSTYYNPSLKSRVTISVDTSKNQFSLKLSSVTAADTAVYYCARHYYYYYLDVWGKGTTVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 57 - protein, 243 aa, synthetic construct
  REGION 1..243: note = HLA-A11 antigen binding domain
EVQLVESGGGLVQPGGSLRLSCAASGFTFSSYWMHWVRQAPGKGLVWVSRINSDGSSTSYADSVKGRFTISRDNAKNTLYLQMNSLRAEDTAVYYCCLGVLLYNWFDPWGQGTLVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 58 - protein, 242 aa, synthetic construct
  REGION 1..242: note = HLA-A11 antigen binding domain
QVQLQESGPGLVKPSQTLSLTCTVSGGSISSGGYYWSWIRQPPGKGLEWIGYIYYSGSTYYNPSLKSRVTISVDTSKNQFSLKLSSVTAADTAVYYCARHYYYYMDVWGKGTTVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 59 - protein, 243 aa, synthetic construct
  REGION 1..243: note = HLA-A11 antigen binding domain
QITLKESGPTLVKPTQTLTLTCTFSGFSLSTSGVGVGWIRQPPGKALEWLALIYWNDDKRYSPSLKSRLTITKDTSKNQVVLTMTNMDPVDTATYYCAHKTTSFYFDYWGQGTLVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 60 - protein, 243 aa, synthetic construct
  REGION 1..243: note = HLA-A11 antigen binding domain
QVQLQESGPGLVKPSQTLSLTCTVSGGSISSGGYYWSWIRQPPGKGLEWIGYIYYSGSTYYNPSLKSRVTISVDTSKNQFSLKLSSVTAADTAVYYCARHYYYYYMDVWGKGTTVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 61-62 - skipped (2 entries)

SEQ ID NO: 63 - protein, 247 aa, synthetic construct
  REGION 1..247: note = HLA-A:02 scFv
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPRTSGGGTKLEIKGGGGSGGGGSGGGGSGGQVQLQQSGPELVKPGASVRISCKASGYTFTSYHIHWVKQRPGQGLEWIGWIYPGNVNTEYNEKFKGKATLTADKSSSTAYMHLSSLTSEDSAVYFCAREEITYAMDYWGQGTSVTVSS

SEQ ID NO: 64 - protein, 247 aa, synthetic construct
  REGION 1..247: note = HLA-A:02 scFv
QVQLVQSGAEVKKPGSSVKVSCKASGYTFTSYHIHWVRQAPGQGLEWMGWIYPGNVNTEYNEKFKGKATITADKSTSTAYMELSSLRSEDTAVYYCAREEITYAMDYWGQGTTVTVSSGGGGSGGGGSGGGGSGGEIVLTQSPGTLSLSPGERATLSCRSSQSIVHSNGNTYLEWYQQKPGQAPRLLIYKVSNRFSGIPDRFSGSGSGTDFTLTISRLEPEDFAVYYCFQGSHVPRTFGGGTKVEIK

SEQ ID NO: 65 - protein, 247 aa, synthetic construct
  REGION 1..247: note = HLA-A:02 scFv
QVQLVQSGAEVKKPGSSVKVSCKASGYTFTSYHIHWVRQAPGQGLEWMGWIYPGNVNTEYNEKFKGKATITADKSTSTAYMELSSLRSEDTAVYYCAREEITYAMDYWGQGTTVTVSSGGGGSGGGGSGGGGSGGDIVMTQTPLSLPVTPGEPASISCRSSQSIVHSNGNTYLEWYLQKPGQSPQLLIYKVSNRFSGVPDRFSGSGSGTDFTLKISRVEAEDVGVYYCFQGSHVPRTFGGGTKVEIK

SEQ ID NO: 66 - protein, 247 aa, synthetic construct
  REGION 1..247: note = HLA-A:02 scFv
EVQLVESGGGLVKPGGSLRLSCAASGYTFTSYHIHWVRQAPGKGLEWVGWIYPGNVNTEYNEKFKGRFTISRDDSKNTLYLQMNSLKTEDTAVYYCAREEITYAMDYWGQGTTVTVSSGGGGSGGGGSGGGGSGGDIQMTQSPSSLSASVGDRVTITCRSSQSIVHSNGNTYLEWYQQKPGKAPKLLIYKVSNRFSGVPSRFSGSGSGTDFTLTISSLQPEDFATYYCFQGSHVPRTFGGGTKVEIK

SEQ ID NO: 67 - protein, 247 aa, synthetic construct
  REGION 1..247: note = HLA-A:02 scFv
QVQLVQSGAEVKKPGSSVKVSCKASGYTFTSYHIHWVRQAPGQGLEWIGWIYPGNVNTEYNEKFKGKATITADESTNTAYMELSSLRSEDTAVYYCAREEITYAMDYWGQGTLVTVSSGGGGSGGGGSGGGGSGGDIQMTQSPSTLSASVGDRVTITCRSSQSIVHSNGNTYLEWYQQKPGKAPKLLIYKVSNRFSGVPARFSGSGSGTEFTLTISSLQPDDFATYYCFQGSHVPRTFGQGTKVEVK

SEQ ID NO: 68 - protein, 247 aa, synthetic construct
  REGION 1..247: note = HLA-A:02 scFv
QVQLVQSGAEVKKPGSSVKVSCKASGYTFTSYHMHWVRQAPGQGLEWIGYIYPGNVNTEYNEKFKGKATLTADKSTNTAYMELSSLRSEDTAVYFCAREEITYAMDYWGQGTLVTVSSGGGGSGGGGSGGGGSGGDVQMTQSPSTLSASVGDRVTITCSSSQSIVHSNGNTYMEWYQQKPGKAPKLLIYKVSNRFSGVPDRFSGSGSGTEFTLTISSLQPDDFATYYCHQGSHVPRTFGQGTKVEVK

SEQ ID NO: 69 - protein, 247 aa, synthetic construct
  REGION 1..247: note = HLA-A:02 scFv
QVQLQQSGPELVKPGASVKMSCKASGYTFTSYHIQWVKQRPGQGLEWIGWIYPGDGSTQYNEKFKGKTTLTADKSSSTAYMLLSSLTSEDSAIYFCAREGTYYAMDYWGQGTSVTVSSGGGGSGGGGSGGGGSGGDVLMTQTPLSLPVSLGDQVSISCRSSQSIVHSNGNTYLEWYLQKPGQSPKLLIYKVSNRFSGVPDRFSGSGSGTDFTLKISRVEAEDLGVYYCFQGSHVPRTFGGGTKLEIK

SEQ ID NO: 70 - protein, 247 aa, synthetic construct
  REGION 1..247: note = HLA-A:02 scFv
QLQLQESGPGLVKPSETLSLTCTVSGYTFTSYHIQWIRQPPGKGLEWIGWIYPGDGSTQYNEKFKGRATISVDTSKNQFSLNLDSVSAADTAIYYCAREGTYYAMDYWGKGSTVTVSSGGGGSGGGGSGGGGSGGDIQMTQSPSSLSASVGDRVTITCRSSQSIVHSNGNTYLEWYQQKPGKAPKLLIYKVSNRFSGVPSRFSGSGSGTDFTFTISSLQPEDIATYYCFQGSHVPRTFGPGTKVDIK

SEQ ID NO: 71 - protein, 247 aa, synthetic construct
  REGION 1..247: note = HLA-A:02 scFv
EVQLVQSGAELKKPGSSVKVSCKASGYTFTSYHIQWVKQAPGQGLEWIGWIYPGDGSTQYNEKFKGKATLTVDKSTNTAYMELSSLRSEDTAVYYCAREGTYYAMDYWGQGTLVTVSSGGGGSGGGGSGGGGSGGDIQMTQSPSTLSASVGDRVTITCRSSQSIVHSNGNTYLEWYQQKPGKAPKLLIYKVSNRFSGVPSRFSGSGSGTDFTLTISSLQPDDFATYYCFQGSHVPRTFGQGTKVEVK

SEQ ID NO: 72 - protein, 247 aa, synthetic construct
  REGION 1..247: note = HLA-A:02 scFv
QVQLVQSGAEVKKPGSSVKVSCKASGYTFTSYHIQWVRQAPGQGLEWMGWIYPGDGSTQYNEKFKGRVTITADKSTSTAYMELSSLRSEDTAVYYCAREGTYYAMDYWGQGTTVTVSSGGGGSGGGGSGGGGSGGEIVLTQSPGTLSLSPGERATLSCRSSQSIVHSNGNTYLEWYQQKPGQAPRLLIYKVSNRFSGIPDRFSGSGSGTDFTLTISRLEPEDFAVYYCFQGSHVPRTFGGGTKVEIK

SEQ ID NO: 73 - protein, 247 aa, synthetic construct
  REGION 1..247: note = HLA-A:02 scFv
QVTLKQSGAEVKKPGSSVKVSCTASGYTFTSYHVSWVRQAPGQGLEWLGRIYPGDGSTQYNEKFKGKVTITADKSMDTSFMELTSLTSEDTAVYYCAREGTYYAMDLWGQGTLVTVSSGGGGSGGGGSGGGGSGGEIVLTQSPGTLSLSPGERATLSCRSSQSIVHSNGNTYLAWYQQKPGQAPRLLISKVSNRFSGVPDRFSGSGSGTDFTLTISRLEPEDFAVYYCQQGSHVPRTFGGGTKVEIK

SEQ ID NO: 74 - protein, 247 aa, synthetic construct
  REGION 1..247: note = HLA-A:02 scFv
QVQLVQSGAEVKKPGASVKVSCKASGYTFTSYHMHWVRQAPGQRLEWMGWIYPGDGSTQYNEKFKGKVTITRDTSASTAYMELSSLRSEDTAVYYCAREGTYYAMDYWGQGTLVTVSSGGGGSGGGGSGGGGSGGDIVMTQTPLSLPVTPGEPASISCRSSQSIVHSNGNTYLDWYLQKPGQSPQLLIYKVSNRFSGVPDRFSGSGSGTDFTLKISRVEAEDVGVYYCMQGSHVPRTFGGGTKVEIK

SEQ ID NO: 75 - DNA, 741 bp, synthetic construct
  misc_feature 1..741: note = HLA-A:02 scFv
  source 1..741: mol_type = other DNA
gatgttttgatgacccaaactccactctccctgcctgtcagtcttggagatcaagcctccatctcttgcagatctagtcagagcattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagtagagtggaggctgaggatctgggagtttattactgctttcaaggttcacatgttcctcggacgtccggtggaggcaccaagctggaaatcaaaggcggaggtggaagcggagggggaggatctggcggcggaggaagcggaggccaggtccagctgcagcagtctggacctgagctggtgaagcctggggcttcagtgaggatatcctgcaaggcttctggctacaccttcacaagttaccatatacattgggtgaagcagaggcctggacagggacttgagtggattggatggatttatcctggaaatgttaatactgagtacaatgagaagttcaagggcaaggccacactgactgcagacaaatcgtccagcacagcctacatgcacctcagcagcctgacctctgaggactctgcggtctatttctgtgccagagaggagattacctatgctatggactactggggtcaaggaacctcagtcaccgtgtcctca

SEQ ID NO: 76 - DNA, 741 bp, synthetic construct
  misc_feature 1..741: note = HLA-A:02 scFv
  source 1..741: mol_type = other DNA
caggtgcagctggtgcagtctggggctgaggtgaagaagcctgggtcctcagtgaaggtttcctgcaaggcttctggatacaccttcactagctatcatatacattgggtgcgccaggcccccggacaagggcttgagtggatgggatggatctaccctggcaatgttaacacagaatataatgagaagttcaagggcaaagccaccattaccgcggacaaatccacgagcacagcctacatggagctgagcagcctgagatctgaagacacggctgtgtattactgtgcgagggaggaaattacctacgctatggactactggggccagggaaccacagtcaccgtgtcctcaggcggaggtggaagcggagggggaggatctggcggcggaggaagcggaggcgagattgtattgacccagagcccaggcaccctgagcctctctccaggagagcgggccaccctcagttgtagatccagtcagagtattgtacacagtaatgggaacacctatttggaatggtatcagcagaaaccaggtcaagccccaagattgctcatctacaaagtctctaacagatttagtggtattccagacaggttcagcggttccggaagtggtactgatttcaccctcacgatctccaggctcgagccagaagatttcgccgtttattactgttttcaaggttcacatgtgccgcgcacattcggtgggggtactaaagtagaaatcaaa

SEQ ID NO: 77 - DNA, 741 bp, synthetic construct
  misc_feature 1..741: note = HLA-A:02 scFv
  source 1..741: mol_type = other DNA
caggtgcagctggtgcagtctggggctgaggtgaagaagcctgggtcctcagtgaaggtttcctgcaaggcttctggatacaccttcactagctatcatatacattgggtgcgccaggcccccggacaagggcttgagtggatgggatggatctaccctggcaatgttaacacagaatataatgagaagttcaagggcaaagccaccattaccgcggacaaatccacgagcacagcctacatggagctgagcagcctgagatctgaagacacggctgtgtattactgtgcgagggaggaaattacctacgctatggactactggggccagggaaccacagtcaccgtgtcctcaggcggaggtggaagcggagggggaggatctggcggcggaggaagcggaggcgacattgtaatgacccagaccccactcagcctgcccgtcactccaggagagccggccagcatcagttgtagatccagtcagagtattgtacacagtaatgggaacacctatttggaatggtatctgcagaaaccaggtcaatccccacaattgctcatctacaaagtctctaacagatttagtggtgtaccagacaggttcagcggttccggaagtggtactgatttcaccctcaagatctccagggtcgaggcagaagatgtcggcgtttattactgttttcaaggttcacatgtgccgcgcacattcggtgggggtactaaagtagaaatcaaa

SEQ ID NO: 78 - DNA, 741 bp, synthetic construct
  misc_feature 1..741: note = HLA-A:02 scFv
  source 1..741: mol_type = other DNA
gaggtgcagctggtggagtctgggggtgggctggtgaagcctgggggctcactgaggctttcctgcgcggcttctggatacaccttcactagctatcatatacattgggtgcgccaggcccccggaaaagggcttgagtgggtgggatggatctaccctggcaatgttaacacagaatataatgagaagttcaagggcagattcaccattagcagggacgattccaagaacacactctacctgcagatgaacagcctgaaaactgaagacacggctgtgtattactgtgcgagggaggaaattacctacgctatggactactggggccagggaaccacagtcaccgtgtcctcaggcggaggtggaagcggagggggaggatctggcggcggaggaagcggaggcgacattcaaatgacccagagcccatccagcctgagcgcatctgtaggtgaccgggtcaccatcacttgtagatccagtcagagtattgtacacagtaatgggaacacctatttggaatggtatcagcagaaaccaggtaaagccccaaaattgctcatctacaaagtctctaacagatttagtggtgtaccaagcaggttcagcggttccggaagtggtactgatttcaccctcacgatctcctctctccagccagaagatttcgccacttattactgttttcaaggttcacatgtgccgcgcacattcggtgggggtactaaagtagaaatcaaa

SEQ ID NO: 79 - DNA, 741 bp, synthetic construct
  misc_feature 1..741: note = HLA-A:02 scFv
  source 1..741: mol_type = other DNA
caggtgcagctggtgcagtctggggctgaggtgaagaagcctgggtcctcagtgaaggtttcctgcaaggcttctggatacaccttcactagctatcatatacattgggtgcgccaggcccccggacaagggcttgagtggatcggatggatctaccctggcaatgttaacacagaatataatgagaagttcaagggcaaagccaccattaccgcggacgaatccacgaacacagcctacatggagctgagcagcctgagatctgaagacacggctgtgtattactgtgcgagggaggaaattacctacgctatggactactggggccagggaaccctggtcaccgtgtcctcaggcggaggtggaagcggagggggaggatctggcggcggaggaagcggaggcgacattcaaatgacccagagcccatccaccctgagcgcatctgtaggtgaccgggtcaccatcacttgtagatccagtcagagtattgtacacagtaatgggaacacctatttggaatggtatcagcagaaaccaggtaaagccccaaaattgctcatctacaaagtctctaacagatttagtggtgtaccagccaggttcagcggttccggaagtggtactgaattcaccctcacgatctcctctctccagccagatgatttcgccacttattactgttttcaaggttcacatgtgccgcgcacattcggtcagggtactaaagtagaagtcaaa

SEQ ID NO: 80 - DNA, 741 bp, synthetic construct
  misc_feature 1..741: note = HLA-A:02 scFv
  source 1..741: mol_type = other DNA
caggtgcagctggtgcagtctggggctgaggtgaagaagcctgggtcctcagtgaaggtttcctgcaaggcttctggatacaccttcactagctatcatatgcattgggtgcgccaggcccccggacaagggcttgagtggatcggatacatctaccctggcaatgttaacacagaatataatgagaagttcaagggcaaagccacccttaccgcggacaaatccacgaacacagcctacatggagctgagcagcctgagatctgaagacacggctgtgtatttctgtgcgagggaggaaattacctacgctatggactactggggccagggaaccctggtcaccgtgtcctcaggcggaggtggaagcggagggggaggatctggcggcggaggaagcggaggcgacgttcaaatgacccagagcccatccaccctgagcgcatctgtaggtgaccgggtcaccatcacttgtagctccagtcagagtattgtacacagtaatgggaacacctatatggaatggtatcagcagaaaccaggtaaagccccaaaattgctcatctacaaagtctctaacagatttagtggtgtaccagacaggttcagcggttccggaagtggtactgaattcaccctcacgatctcctctctccagccagatgatttcgccacttattactgtcatcaaggttcacatgtgccgcgcacattcggtcagggtactaaagtagaagtcaaa

SEQ ID NO: 81 - DNA, 741 bp, synthetic construct
  misc_feature 1..741: note = HLA-A:02 scFv
  source 1..741: mol_type = other DNA
caggtgcagctgcagcagtctgggcctgagctggtgaagcctggggcctcagtgaagatgtcctgcaaggcttctggatacaccttcactagctatcatatccagtgggtgaagcagaggcctggacaagggcttgagtggatcggatggatctaccctggcgatggtagtacacagtataatgagaagttcaagggcaaaaccacccttaccgcggacaaatcctccagcacagcctacatgttgctgagcagcctgacctctgaagactctgctatctatttctgtgcgagggaggggacctactacgctatggactactggggccagggaacctcagtcaccgtgtcctcaggcggaggtggaagcggagggggaggatctggcggcggaggaagcggaggcgatgttttgatgacccagactccactctccctgcctgtctctcttggagaccaagtctccatctcttgtagatccagtcagagtattgtacacagtaatgggaacacctatttagaatggtatctgcagaaaccaggtcagtctccaaagttgctcatctacaaagtctctaacagatttagtggtgtaccagacaggttcagcggttccggaagtggtactgatttcaccctcaagatctcgagagtggaggctgaggatctgggagtttattactgttttcaaggttcacatgtgccgcgcacattcggtggaggtactaaactggaaatcaaa

SEQ ID NO: 82 - DNA, 741 bp, synthetic construct
  misc_feature 1..741: note = HLA-A:02 scFv
  source 1..741: mol_type = other DNA
cagctgcagctgcaggagtctgggcccgggctggtgaagccttcggaaacgctgagcctcacctgcacggtttctggatacaccttcaccagctatcatatccagtggatccgacagccccctggaaaagggcttgagtggatcggatggatctaccctggcgatggttcaacacagtacaatgagaagttcaagggcagagccacgattagcgtggacacatccaagaaccaattctccctgaacctggacagcgtgagtgctgcggacacggccatttattactgtgcgagagagggaacttactacgctatggactactggggcaaagggagcacggtcaccgtgtcctcaggcggaggtggaagcggagggggaggatctggcggcggaggaagcggaggcgacatccagatgacccagagcccaagctccctgagtgcgtccgtgggcgaccgcgtgaccatcacttgcagatcctctcagtccatcgtgcactccaacggcaacacgtacctcgagtggtaccagcagaagcccgggaaggccccgaaactgctcatctacaaggtgagcaaccggttctccggcgtccccagccgcttctcagggtccggctcggggacggatttcaccttcacgattagcagcttgcagcccgaagacatcgccacgtactactgctttcagggaagtcacgtgccgcgtaccttcgggccgggcacgaaagtggatattaag

SEQ ID NO: 83 - DNA, 741 bp, synthetic construct
  misc_feature 1..741: note = HLA-A:02 scFv
  source 1..741: mol_type = other DNA
gaggtgcagctggtgcagtctggggccgagctgaagaagcctgggtcctcggtgaaggtgtcctgcaaggcttctggatacaccttcaccagctatcatatccagtgggtaaaacaggcccctggacaagggcttgagtggatcggatggatctaccctggcgatggttcaacacagtacaatgagaagttcaagggcaaagccacgcttaccgtggacaaatccacgaacacagcctacatggagctgagcagcctgagatctgaggacacggccgtatattactgtgcgagagagggaacttactacgctatggactactggggccaagggaccctggtcaccgtgtcctcaggcggaggtggaagcggagggggaggatctggcggcggaggaagcggaggcgacatccagatgacccagagcccatccaccctgagtgcgtccgtgggcgaccgcgtgaccatcacttgcagatcctctcagtccatcgtgcactccaacggcaacacgtacctcgagtggtaccagcagaagcccgggaaggccccgaaactgctcatctacaaggtgagcaaccggttctccggcgtccccagccgcttctcagggtccggctcggggacggatttcaccctcacgattagcagcttgcagcccgatgacttcgccacgtactactgctttcagggaagtcacgtgccgcgtaccttcgggcagggcacgaaagtggaagttaag

SEQ ID NO: 84 - DNA, 741 bp, synthetic construct
  misc_feature 1..741: note = HLA-A:02 scFv
  source 1..741: mol_type = other DNA
caggtgcagctggtgcagtctggggccgaggtgaagaagcctgggtcctcggtgaaggtgtcctgcaaggcttctggatacaccttcaccagctatcatatccagtgggtacgacaggcccctggacaagggcttgagtggatgggatggatctaccctggcgatggttcaacacagtacaatgagaagttcaagggcagagtcacgattaccgcggacaaatccacgagcacagcctacatggagctgagcagcctgagatctgaggacacggccgtatattactgtgcgagagagggaacttactacgctatggactactggggccaagggaccacggtcaccgtgtcctcaggcggaggtggaagcggagggggaggatctggcggcggaggaagcggaggcgagatcgtcctgacccagagcccagggaccctgagtttgtccccgggcgagcgcgcgaccctcagttgcagatcctctcagtccatcgtgcactccaacggcaacacgtacctcgagtggtaccagcagaagcccgggcaggccccgcgactgctcatctacaaggtgagcaaccggttctccggcatccccgaccgcttctcagggtccggctcggggacggatttcaccctcacgattagccgcttggagcccgaagacttcgccgtgtactactgctttcagggaagtcacgtgccgcgtaccttcggggggggcacgaaagtggaaattaag

SEQ ID NO: 85 - DNA, 741 bp, synthetic construct
  misc_feature 1..741: note = HLA-A:02 scFv
  source 1..741: mol_type = other DNA
caggtgaccctgaagcagtctggggccgaggtgaagaagcctgggtcctcggtgaaggtgtcctgcacggcttctggatacaccttcaccagctatcatgtcagctgggtacgacaggcccctggacaagggcttgagtggttgggaaggatctaccctggcgatggttcaacacagtacaatgagaagttcaagggcaaagtcacgattaccgcggacaaatccatggacacatccttcatggagctgaccagcctgacatctgaggacacggccgtatattactgtgcgagagagggaacttactacgctatggacctctggggccaagggaccctggtcaccgtgtcctcaggcggaggtggaagcggagggggaggatctggcggcggaggaagcggaggcgagatcgtcctgacccagagcccagggaccctgagtttgtccccgggcgagcgcgcgaccctcagttgcagatcctctcagtccatcgtgcactccaacggcaacacgtacctcgcgtggtaccagcagaagcccgggcaggccccgcgactgctcatctccaaggtgagcaaccggttctccggcgtccccgaccgcttctcagggtccggctcggggacggatttcaccctcacgattagccgcttggagcccgaagacttcgccgtgtactactgccaacagggaagtcacgtgccgcgtaccttcggggggggcacgaaagtggaaattaag

SEQ ID NO: 86 - DNA, 741 bp, synthetic construct
  misc_feature 1..741: note = HLA-A:02 scFv
  source 1..741: mol_type = other DNA
caggtgcagctggtgcagtctggggccgaggtgaagaagcctggggcctcggtgaaggtgtcctgcaaggcttctggatacaccttcaccagctatcatatgcactgggtacgacaggcccctggacaaaggcttgagtggatgggatggatctaccctggcgatggttcaacacagtacaatgagaagttcaagggcaaagtcacgattacccgggacacatccgcgagcacagcctacatggagctgagcagcctgagatctgaggacacggccgtatattactgtgcgagagagggaacttactacgctatggactactggggccaagggaccctggtcaccgtgtcctcaggcggaggtggaagcggagggggaggatctggcggcggaggaagcggaggcgacatcgtcatgacccagaccccactgtccctgcctgtgaccccgggcgagcccgcgagcatcagttgcagatcctctcagtccatcgtgcactccaacggcaacacgtacctcgactggtacctgcagaagcccgggcagtccccgcaactgctcatctacaaggtgagcaaccggttctccggcgtccccgaccgcttctcagggtccggctcggggacggatttcaccctcaagattagccgcgtggaggccgaagacgtcggcgtgtactactgcatgcagggaagtcacgtgccgcgtaccttcggggggggcacgaaagtggaaattaag

SEQ ID NO: 87 - protein, 16 aa, synthetic construct
  REGION 1..16: note = CDR-L1
RSSQSIVHSNGNTYLE

SEQ ID NO: 88 - protein, 16 aa, synthetic construct
  REGION 1..16: note = CDR-L1
SSSQSIVHSNGNTYME

SEQ ID NO: 89 - protein, 16 aa, synthetic construct
  REGION 1..16: note = CDR-L1
RSSQSIVHSNGNTYLD

SEQ ID NO: 90 - protein, 12 aa, synthetic construct
  REGION 1..12: note = CDR-L2
KVSNRFSGVPDR

SEQ ID NO: 91 - protein, 12 aa, synthetic construct
  REGION 1..12: note = CDR-L2
KVSNRFSGVPAR

SEQ ID NO: 92 - protein, 9 aa, synthetic construct
  REGION 1..9: note = CDR-L3
FQGSHVPRT

SEQ ID NO: 93 - protein, 9 aa, synthetic construct
  REGION 1..9: note = CDR-L3
HQGSHVPRT

SEQ ID NO: 94 - protein, 9 aa, synthetic construct
  REGION 1..9: note = CDR-L3
MQGSHVPRT

SEQ ID NO: 95 - protein, 12 aa, synthetic construct
  REGION 1..12: note = CDR-H1
ASGYTFTSYHIH

SEQ ID NO: 96 - protein, 12 aa, synthetic construct
  REGION 1..12: note = CDR-H1
ASGYTFTSYHMH

SEQ ID NO: 97 - protein, 11 aa, synthetic construct
  REGION 1..11: note = CDR-H1
SGYTFTSYHMH

SEQ ID NO: 98 - protein, 18 aa, synthetic construct
  REGION 1..18: note = CDR-H2
WIYPGNVNTEYNEKFKGK

SEQ ID NO: 99 - protein, 18 aa, synthetic construct
  REGION 1..18: note = CDR-H2
YIYPGNVNTEYNEKFKGK

SEQ ID NO: 100 - protein, 17 aa, synthetic construct
  REGION 1..17: note = CDR-H2
WIYPGDGSTQYNEKFKG

SEQ ID NO: 101 - protein, 9 aa, synthetic construct
  REGION 1..9: note = CDR-H3
EEITYAMDY

SEQ ID NO: 102 - protein, 9 aa, synthetic construct
  REGION 1..9: note = CDR-H3
EGTYYAMDY

SEQ ID NO: 103 - protein, 10 aa, synthetic construct
  REGION 1..10: note = LB-CYBA-1Y
  SITE 10: note = Xaa is Tyr or His
STMERWGQKX

SEQ ID NO: 104 - protein, 11 aa, synthetic construct
  REGION 1..11: note = LB-OAS1-1R
  SITE 7: note = Xaa is Arg or Thr
ETDDPRXYQKY

SEQ ID NO: 105 - protein, 9 aa, synthetic construct
  REGION 1..9: note = HA-1/A2
  SITE 3: note = Xaa is His or Arg
VLXDDLLEA

SEQ ID NO: 106 - protein, 9 aa, synthetic construct
  REGION 1..9: note = HA-2
  SITE 9: note = Xaa is Val or Met
YIGEVLVSX

SEQ ID NO: 107 - protein, 9 aa, synthetic construct
  REGION 1..9: note = HA-8
  SITE 1: note = Xaa is Arg or Pro
XTLDKVLEV

SEQ ID NO: 108 - protein, 9 aa, synthetic construct
  REGION 1..9: note = HA-3
  SITE 2: note = Xaa is Thr or Met
VXEPGTAQY

SEQ ID NO: 109 - protein, 11 aa, synthetic construct
  REGION 1..11: note = HwA11-S
  SITE 6: note = Xaa is Ser or Thr
CIPPDXLLFPA

SEQ ID NO: 110 - protein, 11 aa, synthetic construct
  REGION 1..11: note = LB-ADIR-1F
  SITE 9: note = Xaa is Phe or Ser
SVAPALALXPA

SEQ ID NO: 111 - protein, 9 aa, synthetic construct
  REGION 1..9: note = LB-HIVEP1-1S
  SITE 6: note = Xaa is Ser or Asn
SLPKHXVTI

SEQ ID NO: 112 - protein, 9 aa, synthetic construct
  REGION 1..9: note = LB-NISCH-1A
  SITE 7: note = Xaa is Ala or Val
ALAPAPXEV

SEQ ID NO: 113 - protein, 9 aa, synthetic construct
  REGION 1..9: note = LB-SSR1-1S
  SITE 1: note = Xaa is Ser or Leu
XLAVAQDLT

SEQ ID NO: 114 - protein, 10 aa, synthetic construct
  REGION 1..10: note = LB-WNK1-1I
  SITE 7: note = Xaa is Ile or Met
RTLSPEXITV

SEQ ID NO: 115 - protein, 10 aa, synthetic construct
  REGION 1..10: note = T4A
  SITE 10: note = Xaa is Ala or Glu
GLYTYWSAGX

SEQ ID NO: 116 - protein, 9 aa, synthetic construct
  REGION 1..9: note = UTA2-1
  SITE 3: note = Xaa is Leu or Pro
QLXNSVLTL

SEQ ID NO: 117 - protein, 9 aa, synthetic construct
  REGION 1..9: note = LB-CLYBL-1Y
  SITE 5: note = Xaa is Tyr or Asp
SLAAXIPRL

SEQ ID NO: 118 - protein, 10 aa, synthetic construct
  REGION 1..10: note = TRIM22
  SITE 7: note = Xaa is Cys or Arg
MAVPPCXIGV

SEQ ID NO: 119 - protein, 11 aa, synthetic construct
  REGION 1..11: note = PARP10-1L
  SITE 3: note = Xaa is Leu or Pro
GLXGQEGLVEI

SEQ ID NO: 120 - protein, 9 aa, synthetic construct
  REGION 1..9: note = FAM119A-1T
  SITE 8: note = Xaa is Thr or Ile
AMLERQFXV

SEQ ID NO: 121 - protein, 9 aa, synthetic construct
  REGION 1..9: note = GLRX3-1S
  SITE 3: note = Xaa is Ser or Pro
FLXSANEHL

SEQ ID NO: 122 - protein, 9 aa, synthetic construct
  REGION 1..9: note = HNF4G-1M
  SITE 2: note = Xaa is Met or Ile
MXYKDILLL

SEQ ID NO: 123 - protein, 9 aa, synthetic construct
  REGION 1..9: note = HMMR-1V
  SITE 6: note = Xaa is Val or Ala
SLQEKXAKA

SEQ ID NO: 124 - protein, 9 aa, synthetic construct
  REGION 1..9: note = BCL2A1
  SITE 4: note = Xaa is Asn or Lys
VLQXVAFSV

SEQ ID NO: 125 - protein, 10 aa, synthetic construct
  REGION 1..10: note = CDC26-1F
  SITE 1: note = Xaa is Phe or Ser
XVAGTQEVFV

SEQ ID NO: 126 - protein, 10 aa, synthetic construct
  REGION 1..10: note = APOBEC3F-1S/A
  SITE 3: note = Xaa is Ser or Ala
FLXEHPNVTL

SEQ ID NO: 127 - protein, 9 aa, synthetic construct
  REGION 1..9: note = LB-PRCP-1D
  SITE 8: note = Xaa is Asp or Glu
FMWDVAEXL

SEQ ID NO: 128 - protein, 11 aa, synthetic construct
  REGION 1..11: note = LB-PRCP-1D
  SITE 8: note = Xaa is Asp or Glu
FMWDVAEXLKA

SEQ ID NO: 129 - protein, 9 aa, synthetic construct
  REGION 1..9: note = LB-CCL4-1T
  SITE 7: note = Xaa is Thr or Ser
CADPSEXWV

SEQ ID NO: 130 - protein, 9 aa, synthetic construct
  REGION 1..9: note = LB-NCAPD3-1Q
  SITE 3: note = Xaa is Gln or Arg
WLXGVVPVV

SEQ ID NO: 131 - protein, 9 aa, synthetic construct
  REGION 1..9: note = LB-NDC80-1P
  SITE 7: note = Xaa is Pro or Ala
HLEEQIXKV

SEQ ID NO: 132 - protein, 9 aa, synthetic construct
  REGION 1..9: note = LB-TTK-1D
  SITE 4: note = Xaa is Asp or Glu
RLHXGRVFV

SEQ ID NO: 133 - protein, 9 aa, synthetic construct
  REGION 1..9: note = WDR27-1L
  SITE 2: note = Xaa is Leu or Pro
SXDDHVVAV

SEQ ID NO: 134 - protein, 11 aa, synthetic construct
  REGION 1..11: note = MIIP
  SITE 8: note = Xaa is Lys or Glu
SEESAVPXRSW

SEQ ID NO: 135 - protein, 10 aa, synthetic construct
  REGION 1..10: note = MIIP
  SITE 7: note = Xaa is Lys or Glu
EESAVPXRSW

SEQ ID NO: 136 - protein, 9 aa, synthetic construct
  REGION 1..9: note = LB-DHX33-1C
  SITE 9: note = Xaa is Cys or Arg
YLYEGGISX

SEQ ID NO: 137 - protein, 10 aa, synthetic construct
  REGION 1..10: note = PANE1
RVWDLPGVLK

SEQ ID NO: 138 - protein, 10 aa, synthetic construct
  REGION 1..10: note = SP110
  SITE 4: note = Xaa is Arg or Gly
SLPXGTSTPK

SEQ ID NO: 139 - protein, 9 aa, synthetic construct
  REGION 1..9: note = ACC-1C/Y
  SITE 5: note = Xaa is Tyr or Cys
DYLQXVLQI

SEQ ID NO: 140 - protein, 9 aa, synthetic construct
  REGION 1..9: note = P2RX7
  SITE 6: note = Xaa is His or Arg
WFHHCXPKY

SEQ ID NO: 141 - protein, 9 aa, synthetic construct
  REGION 1..9: note = ACC-4
  SITE 9: note = Xaa is Arg or Gly
ATLPLLCAX

SEQ ID NO: 142 - protein, 10 aa, synthetic construct
  REGION 1..10: note = ACC-5
  SITE 10: note = Xaa is Arg or Gly
WATLPLLCAX

SEQ ID NO: 143 - protein, 9 aa, synthetic construct
  REGION 1..9: note = AKAP13
  SITE 9: note = Xaa is Met or Thr
APAGVREVX

SEQ ID NO: 144 - protein, 10 aa, synthetic construct
  REGION 1..10: note = LB-APOBEC3B-1K
  SITE 1: note = Xaa is Lys or Glu
XPQYHAEMCF

SEQ ID NO: 145 - protein, 9 aa, synthetic construct
  REGION 1..9: note = APOBEC3H
  SITE 5: note = Xaa is Lys or Glu
KPQQXGLRL

SEQ ID NO: 146 - protein, 9 aa, synthetic construct
  REGION 1..9: note = LB-ARHGDIB-1R
  SITE 7: note = Xaa is Arg or Pro
LPRACWXEA

SEQ ID NO: 147 - protein, 11 aa, synthetic construct
  REGION 1..11: note = LB-BCAT2-1R
  SITE 3: note = Xaa is Arg or Thr
QPXRALLFVIL

SEQ ID NO: 148 - protein, 11 aa, synthetic construct
  REGION 1..11: note = BFAR
  SITE 11: note = Xaa is Met or Arg
APNTGRANQQX

SEQ ID NO: 149 - protein, 11 aa, synthetic construct
  REGION 1..11: note = C14orf169
  SITE 4: note = Xaa is Ala or Val
RPRXPTEELAL

SEQ ID NO: 150 - protein, 11 aa, synthetic construct
  REGION 1..11: note = LB-C16ORF-1R
  SITE 1: note = Xaa is Arg or Trp
XPCPSVGLSFL

SEQ ID NO: 151 - protein, 9 aa, synthetic construct
  REGION 1..9: note = C18orf21
  SITE 6: note = Xaa is Ala or Thr
NPATPXSKL

SEQ ID NO: 152 - protein, 10 aa, synthetic construct
  REGION 1..10: note = LB-EBI3-1I
  SITE 8: note = Xaa is Ile or Val
RPRARYYXQV

SEQ ID NO: 153 - protein, 9 aa, synthetic construct
  REGION 1..9: note = POP1
  SITE 7: note = Xaa is Asn or Lys
LPQKKSXAL

SEQ ID NO: 154 - protein, 9 aa, synthetic construct
  REGION 1..9: note = SCRIB
  SITE 7: note = Xaa is Leu or Pro
LPQQPPXSL

SEQ ID NO: 155 - protein, 9 aa, synthetic construct
  REGION 1..9: note = MTRR
  SITE 5: note = Xaa is Ser or Leu
SPASXRTDL

SEQ ID NO: 156 - protein, 9 aa, synthetic construct
  REGION 1..9: note = LLGL2
  SITE 5: note = Xaa is Arg or His
SPSLXILAI

SEQ ID NO: 157 - protein, 11 aa, synthetic construct
  REGION 1..11: note = LB-ECGF-1H
  SITE 3: note = Xaa is His or Arg
RPXAIRRPLAL

SEQ ID NO: 158 - protein, 11 aa, synthetic construct
  REGION 1..11: note = LB-ERAP1-1R
  SITE 3: note = Xaa is Arg or Pro
HPXQEQIALLA

SEQ ID NO: 159 - protein, 9 aa, synthetic construct
  REGION 1..9: note = LB-ERAP1-1R
  SITE 3: note = Xaa is Arg or Pro
HPXQEQIAL

SEQ ID NO: 160 - protein, 9 aa, synthetic construct
  REGION 1..9: note = LB-FUCA2-1V
  SITE 5: note = Xaa is Val or Met
RLRQXGSWL

SEQ ID NO: 161 - protein, 9 aa, synthetic construct
  REGION 1..9: note = LB-GEMIN4-1V
  SITE 9: note = Xaa is Val or Glu
FPALRFVEX

SEQ ID NO: 162 - protein, 11 aa, synthetic construct
  REGION 1..11: note = HDGF
  SITE 11: note = Xaa is Leu or Pro
LPMEVEKNSTX

SEQ ID NO: 163 - protein, 11 aa, synthetic construct
  REGION 1..11: note = LB-PDCD11-1F
  SITE 8: note = Xaa is Phe or Leu
GPDSSKTXLCL

SEQ ID NO: 164 - protein, 9 aa, synthetic construct
  REGION 1..9: note = LB-PFAS-1P
  SITE 2: note = Xaa is Pro or Ser
AXGHTRRKL

SEQ ID NO: 165 - protein, 10 aa, synthetic construct
  REGION 1..10: note = LB-TEP1-1S
  SITE 9: note = Xaa is Ser or Pro
APDGAKVAXL

SEQ ID NO: 166 - protein, 11 aa, synthetic construct
  REGION 1..11: note = LB-TMEM8A-1I
  SITE 7: note = Xaa is Ile or Val
RPRSVTXQPLL

SEQ ID NO: 167 - protein, 10 aa, synthetic construct
  REGION 1..10: note = LB-USP15-1I
  SITE 8: note = Xaa is Ile or Thr
MPSHLRNXLL

SEQ ID NO: 168 - protein, 9 aa, synthetic construct
  REGION 1..9: note = LRH-1
TPNQRQNVC

SEQ ID NO: 169 - protein, 9 aa, synthetic construct
  REGION 1..9: note = LB-MOB3A-1C
  SITE 1: note = Xaa is Cys or Ser
XPRPGTWTC

SEQ ID NO: 170 - protein, 11 aa, synthetic construct
  REGION 1..11: note = LB-ZDHHC6-1Y
  SITE 4: note = Xaa is Tyr or His
RPRXWILLVKI

SEQ ID NO: 171 - protein, 10 aa, synthetic construct
  REGION 1..10: note = ZAPHIR
IPRDSWWVEL

SEQ ID NO: 172 - protein, 9 aa, synthetic construct
  REGION 1..9: note = HEATR1
  SITE 7: note = Xaa is Glu or Gly
ISKERAXAL

SEQ ID NO: 173 - protein, 9 aa, synthetic construct
  REGION 1..9: note = LB-GSTP1-1V
  SITE 7: note = Xaa is Val or Ile
DLRCKYXSL

SEQ ID NO: 174 - protein, 9 aa, synthetic construct
  REGION 1..9: note = HA-1/B60
  SITE 6: note = Xaa is His or Arg
KECVLXDDL

SEQ ID NO: 175 - protein, 9 aa, synthetic construct
  REGION 1..9: note = LB-SON-1R
  SITE 6: note = Xaa is Arg or Cys
SETKQXTVL

SEQ ID NO: 176 - protein, 9 aa, synthetic construct
  REGION 1..9: note = LB-SWAP70-1Q
  SITE 6: note = Xaa is Gln or Glu
MEQLEXLEL

SEQ ID NO: 177 - protein, 9 aa, synthetic construct
  REGION 1..9: note = LB-TRIP10-1EPC
  SITE 2: note = Xaa is Glu or Gly
  SITE 3: note = Xaa is Pro or Ser
  SITE 7: note = Xaa is Cys or Gly
GXXQDLXTL

SEQ ID NO: 178 - protein, 9 aa, synthetic construct
  REGION 1..9: note = LB-NUP133-1R
  SITE 8: note = Xaa is Arg or Gln
SEDLILCXL

SEQ ID NO: 179 - protein, 11 aa, synthetic construct
  REGION 1..11: note = LB-ZNFX1-1Q
  SITE 8: note = Xaa is Gln or His
NEIEDVWXLDL

SEQ ID NO: 180 - protein, 11 aa, synthetic construct
  REGION 1..11: note = SLC1A5
  SITE 3: note = Xaa is Ala or Pro
AEXTANGGLAL

SEQ ID NO: 181 - protein, 10 aa, synthetic construct
  REGION 1..10: note = ACC-2
  SITE 6: note = Xaa is Asp or Gly
KEFEDXIINW

SEQ ID NO: 182 - protein, 11 aa, synthetic construct
  REGION 1..11: note = ACC-6
MEIFIEVFSHF

SEQ ID NO: 183 - protein, 10 aa, synthetic construct
  REGION 1..10: note = HB-1H/Y
  SITE 8: note = Xaa is His or Tyr
EEKRGSLXVW

SEQ ID NO: 184 - protein, 9 aa, synthetic construct
  REGION 1..9: note = DPH1
  SITE 2: note = Xaa is Val or Leu
SXLPEVDVW

SEQ ID NO: 185 - protein, 9 aa, synthetic construct
  REGION 1..9: note = UGT2B17/A02
CVATMIFMI

SEQ ID NO: 186 - protein, 10 aa, synthetic construct
  REGION 1..10: note = UGT2B17/A29
AELLNIPFLY

SEQ ID NO: 187 - protein, 10 aa, synthetic construct
  REGION 1..10: note = UGT2B17/B44
AELLNIPFLY

SEQ ID NO: 188 - protein, 9 aa, synthetic construct
  REGION 1..9: note = DFFRY
  SITE 4: note = Xaa is Cys or Ser
IVDXLTEMY

SEQ ID NO: 189 - protein, 9 aa, synthetic construct
  REGION 1..9: note = SMCY
FIDSYICQV

SEQ ID NO: 190 - protein, 11 aa, synthetic construct
  REGION 1..11: note = TMSB4Y
EVLLRPGLHFR

SEQ ID NO: 191 - protein, 11 aa, synthetic construct
  REGION 1..11: note = SMCY
  SITE 3: note = Xaa is Ser or Ala
  SITE 8: note = Xaa is Arg or Gln
SPXVDKAXAEL

SEQ ID NO: 192 - protein, 8 aa, synthetic construct
  REGION 1..8: note = UTY
  SITE 5: note = Xaa is His or Arg
  SITE 7: note = Xaa is Asp or Asn
LPHNXTXL

SEQ ID NO: 193 - protein, 9 aa, synthetic construct
  REGION 1..9: note = RPS4Y
  SITE 8: note = Xaa is Val or Leu
TIRYPDPXI

SEQ ID NO: 194 - protein, 10 aa, synthetic construct
  REGION 1..10: note = UTY
  SITE 1: note = Xaa is Arg or Gly
  SITE 6: note = Xaa is Glu or Ala
  SITE 8: note = Xaa is Val or Pro
XESEEXSXSL

SEQ ID NO: 195 - protein, 9 aa, synthetic construct
  REGION 1..9: note = HA-1
VLHDDLLEA

SEQ ID NO: 196 - protein, 19 aa, synthetic construct
  REGION 1..19: note = P2A peptide
ATNFSLLKQAGDVEENPGP

SEQ ID NO: 197 - protein, 608 aa, synthetic construct
  REGION 1..608: note = C-003754 KP7 HA-1H TCRalpha T48C P2A KP7 HA-1H TCRbeta S57C
MVKIRQFLLAILWLQLSCVSAAKNEVEQSPQNLTAQEGEFITINCSYSVGISALHWLQQHPGGGIVSLFMLSSGKKKHGRLIATINIQEKHSSLHITASHPRDSAVYICAVRSVSGAGSYQLTFGKGTKLSVIPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGATNFSLLKQAGDVEENPGPMGTSLLCWMALCLLGADHADTGVSQNPRHKITKRGQNVTFRCDPISEHNRLYWYRQTLGQGPEFLTYFQNEAQLEKSRLLSDRFSAERPKGSFSTLEIQRTEQGDSAMYLCASSIDSFNEQFFGPGTRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 198 - protein, 250 aa, synthetic construct
  REGION 1..250: note = HA-1H TCR alpha
MVKIRQFLLAILWLQLSCVSAAKNEVEQSPQNLTAQEGEFITINCSYSVGISALHWLQQHPGGGIVSLFMLSSGKKKHGRLIATINIQEKHSSLHITASHPRDSAVYICAVRSVSGAGSYQLTFGKGTKLSVIPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLS

SEQ ID NO: 199 - protein, 276 aa, synthetic construct
  REGION 1..276: note = HA-1(H) TCRbeta
MGTSLLCWMALCLLGADHADTGVSQNPRHKITKRGQNVTFRCDPISEHNRLYWYRQTLGQGPEFLTYFQNEAQLEKSRLLSDRFSAERPKGSFSTLEIQRTEQGDSAMYLCASSIDSFNEQFFGPGTRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLS

SEQ ID NO: 200 - protein, 440 aa, synthetic construct
  REGION 1..440: note = C-003755 KP7 HA-1H FTCRalpha LIR1 TICD
MVKIRQFLLAILWLQLSCVSAAKNEVEQSPQNLTAQEGEFITINCSYSVGISALHWLQQHPGGGIVSLFMLSSGKKKHGRLIATINIQEKHSSLHITASHPRDSAVYICAVRSVSGAGSYQLTFGKGTKLSVIPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 201 - protein, 466 aa, synthetic construct
  REGION 1..466: note = C-003756 KP7 HA-1H FTCRbeta LIR1 TICD
MGTSLLCWMALCLLGADHADTGVSQNPRHKITKRGQNVTFRCDPISEHNRLYWYRQTLGQGPEFLTYFQNEAQLEKSRLLSDRFSAERPKGSFSTLEIQRTEQGDSAMYLCASSIDSFNEQFFGPGTRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 202 - DNA, 1827 bp, synthetic construct
  misc_feature 1..1827: note = C-003754 KP7 HA-1H TCRalpha T48C P2A KP7 HA-1H TCRbeta S57C
  source 1..1827: mol_type = other DNA
atggtgaagatccggcaatttttgttggctattttgtggcttcagctaagctgtgtaagtgccgccaaaaatgaagtggagcagagtcctcagaacctgactgcccaggaaggagaatttatcacaatcaactgcagttactcggtaggaataagtgccttacactggctgcaacagcatccaggaggaggcattgtttccttgtttatgctgagctcagggaagaagaagcatggaagattaattgccacaataaacatacaggaaaagcacagctccctgcacatcacagcctcccatcccagagactctgccgtctacatctgtgctgtcagaagcgtgtccggggccggctcctaccagctcacctttgggaaggggaccaaattatcagtcattccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaatgtgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagcggatccggagccaccaacttcagcctgctgaagcaggccggcgacgtggaggagaaccccggccccatgggcaccagcctcctctgctggatggccctgtgtctcctgggggcagatcacgcagatactggagtctcccagaaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcagcatcgactccttcaacgagcagttcttcgggccgggcaccaggctcacggtcctcgaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtctgcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 203 - DNA, 750 bp, synthetic construct
  misc_feature 1..750: note = HA-1H TCR alpha
  source 1..750: mol_type = other DNA
atggtgaagatccggcaatttttgttggctattttgtggcttcagctaagctgtgtaagtgccgccaaaaatgaagtggagcagagtcctcagaacctgactgcccaggaaggagaatttatcacaatcaactgcagttactcggtaggaataagtgccttacactggctgcaacagcatccaggaggaggcattgtttccttgtttatgctgagctcagggaagaagaagcatggaagattaattgccacaataaacatacaggaaaagcacagctccctgcacatcacagcctcccatcccagagactctgccgtctacatctgtgctgtcagaagcgtgtccggggccggctcctaccagctcacctttgggaaggggaccaaattatcagtcattccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaatgtgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtca

SEQ ID NO: 204 - DNA, 828 bp, synthetic construct
  misc_feature 1..828: note = HA-1H TCR beta
  source 1..828: mol_type = other DNA
atgggcaccagcctcctctgctggatggccctgtgtctcctgggggcagatcacgcagatactggagtctcccagaaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcagcatcgactccttcaacgagcagttcttcgggccgggcaccaggctcacggtcctcgaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtctgcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtct

SEQ ID NO: 205 - DNA, 1279 bp, synthetic construct
  misc_feature 1..1279: note = C-003755 KP7 HA-1H FTCRalpha LIR1 TICD
  source 1..1279: mol_type = other DNA
atggtgaagatccggcaatttttgttggctattttgtggcttcagctaagctgtgtaagtgccgccaaaaatgaagtggagcagagtcctcagaacctgactgcccaggaaggagaatttatcacaatcaactgcagttactcggtaggaataagtgccttacactggctgcaacagcatccaggaggaggcattgtttccttgtttatgctgagctcagggaagaagaagcatggaagattaattgccacaataaacatacaggaaaagcacagctccctgcacatcacagcctcccatcccagagactctgccgtctacatctgtgctgtcagaagcgtgtccggggccggctcctaccagctcacctttgggaaggggaccaaattatcagtcattccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaatgtgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagttgtgatcggcatcttggtggccgtcatcctactgctcctcctcctcctcctcctcttcctcatcctccgacatcgacgtcagggcaaacactggacatcgacccagagaaaggctgatttccaacatcctgcaggggctgtggggccagagcccacagacagaggcctgcagtggaggtccagcccagctgccgatgcccaggaagaaaacctctatgctgccgtgaagcacacacagcctgaggatggggtggagatggacactcggagcccacacgatgaagacccccaggcagtgacgtatgccgaggtgaaacactccagacctaggagagaaatggcctctcctccttccccactgtctggggaattcctggacacaaaggacagacaggcggaagaggacaggcagatggacactgaggctgctgcatctgaagccccccaggatgtgacctacgcccagctgcacagcttgaccctcagacgggaggcaactgagcctcctccatcccaggaagggccct

SEQ ID NO: 206 - DNA, 1401 bp, synthetic construct
  misc_feature 1..1401: note = C-003756 KP7 HA-1H FTCRbeta LIR1 TICD
  source 1..1401: mol_type = other DNA
atgggcaccagcctcctctgctggatggccctgtgtctcctgggggcagatcacgcagatactggagtctcccagaaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcagcatcgactccttcaacgagcagttcttcgggccgggcaccaggctcacggtcctcgaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtctgcacagacccgcagcccctcaaggagcagcccgccctcaatgactccagatactgcctgagcagccgcctgagggtgtcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgttgtgatcggcatcttggtggccgtcatcctactgctcctcctcctcctcctcctcttcctcatcctccgacatcgacgtcagggcaaacactggacatcgacccagagaaaggctgatttccaacatcctgcaggggctgtggggccagagcccacagacagaggcctgcagtggaggtccagcccagctgccgatgcccaggaagaaaacctctatgctgccgtgaagcacacacagcctgaggatggggtggagatggacactcggagcccacacgatgaagacccccaggcagtgacgtatgccgaggtgaaacactccagacctaggagagaaatggcctctcctccttccccactgtctggggaattcctggacacaaaggacagacaggcggaagaggacaggcagatggacactgaggctgctgcatctgaagccccccaggatgtgacctacgcccagctgcacagcttgaccctcagacgggaggcaactgagcctcctccatcccaggaagggccctctccagctgtgcccagcatctacgccactctggccatccactag

SEQ ID NO: 207 - protein, 269 aa, synthetic construct
  REGION 1..269: note = HLA-A:02 scFv
MDMRVPAQLLGLLLLWLRGARCDVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPRTSGGGTKLEIKGGGGSGGGGSGGGGSGGQVQLQQSGPELVKPGASVRISCKASGYTFTSYHIHWVKQRPGQGLEWIGWIYPGNVNTEYNEKFKGKATLTADKSSSTAYMHLSSLTSEDSAVYFCAREEITYAMDYWGQGTSVTVSS

SEQ ID NO: 208 - protein, 807 aa, synthetic construct
  REGION 1..807: note = HLA-A:02 scFv
ATGGACATGAGGGTCCCCGCTCAGCTCCTGGGGCTCCTGCTACTCTGGCTCCGAGGTGCCAGATGTGATGTTTTGATGACCCAAACTCCACTCTCCCTGCCTGTCAGTCTTGGAGATCAAGCCTCCATCTCTTGCAGATCTAGTCAGAGCATTGTACATAGTAATGGAAACACCTATTTAGAATGGTACCTGCAGAAACCAGGCCAGTCTCCAAAGCTCCTGATCTACAAAGTTTCCAACCGATTTTCTGGGGTCCCAGACAGGTTCAGTGGCAGTGGATCAGGGACAGATTTCACACTCAAGATCAGTAGAGTGGAGGCTGAGGATCTGGGAGTTTATTACTGCTTTCAAGGTTCACATGTTCCTCGGACGTCCGGTGGAGGCACCAAGCTGGAAATCAAAGGCGGAGGTGGAAGCGGAGGGGGAGGATCTGGCGGCGGAGGAAGCGGAGGCCAGGTCCAGCTGCAGCAGTCTGGACCTGAGCTGGTGAAGCCTGGGGCTTCAGTGAGGATATCCTGCAAGGCTTCTGGCTACACCTTCACAAGTTACCATATACATTGGGTGAAGCAGAGGCCTGGACAGGGACTTGAGTGGATTGGATGGATTTATCCTGGAAATGTTAATACTGAGTACAATGAGAAGTTCAAGGGCAAGGCCACACTGACTGCAGACAAATCGTCCAGCACAGCCTACATGCACCTCAGCAGCCTGACCTCTGAGGACTCTGCGGTCTATTTCTGTGCCAGAGAGGAGATTACCTATGCTATGGACTACTGGGGTCAAGGAACCTCAGTCACCGTGTCCTCA

SEQ ID NO: 209 - protein, 269 aa, synthetic construct
  REGION 1..269: note = HLA-A:02 scFv
MDMRVPAQLLGLLLLWLRGARCQVQLVQSGAEVKKPGSSVKVSCKASGYTFTSYHIHWVRQAPGQGLEWIGWIYPGNVNTEYNEKFKGKATITADESTNTAYMELSSLRSEDTAVYYCAREEITYAMDYWGQGTLVTVSSGGGGSGGGGSGGGGSGGDIQMTQSPSTLSASVGDRVTITCRSSQSIVHSNGNTYLEWYQQKPGKAPKLLIYKVSNRFSGVPARFSGSGSGTEFTLTISSLQPDDFATYYCFQGSHVPRTFGQGTKVEVK

SEQ ID NO: 210 - protein, 807 aa, synthetic construct
  REGION 1..807: note = HLA-A:02 scFv
ATGGACATGAGGGTCCCCGCTCAGCTCCTGGGGCTCCTGCTACTCTGGCTCCGAGGTGCCAGATGTCAGGTGCAGCTGGTGCAGTCTGGGGCTGAGGTGAAGAAGCCTGGGTCCTCAGTGAAGGTTTCCTGCAAGGCTTCTGGATACACCTTCACTAGCTATCATATACATTGGGTGCGCCAGGCCCCCGGACAAGGGCTTGAGTGGATCGGATGGATCTACCCTGGCAATGTTAACACAGAATATAATGAGAAGTTCAAGGGCAAAGCCACCATTACCGCGGACGAATCCACGAACACAGCCTACATGGAGCTGAGCAGCCTGAGATCTGAAGACACGGCTGTGTATTACTGTGCGAGGGAGGAAATTACCTACGCTATGGACTACTGGGGCCAGGGAACCCTGGTCACCGTGTCCTCAGGCGGAGGTGGAAGCGGAGGGGGAGGATCTGGCGGCGGAGGAAGCGGAGGCGACATTCAAATGACCCAGAGCCCATCCACCCTGAGCGCATCTGTAGGTGACCGGGTCACCATCACTTGTAGATCCAGTCAGAGTATTGTACACAGTAATGGGAACACCTATTTGGAATGGTATCAGCAGAAACCAGGTAAAGCCCCAAAATTGCTCATCTACAAAGTCTCTAACAGATTTAGTGGTGTACCAGCCAGGTTCAGCGGTTCCGGAAGTGGTACTGAATTCACCCTCACGATCTCCTCTCTCCAGCCAGATGATTTCGCCACTTATTACTGTTTTCAAGGTTCACATGTGCCGCGCACATTCGGTCAGGGTACTAAAGTAGAAGTCAAA

SEQ ID NO: 211 - protein, 269 aa, synthetic construct
  REGION 1..269: note = HLA-A:02 scFv
MDMRVPAQLLGLLLLWLRGARCQVQLVQSGAEVKKPGSSVKVSCKASGYTFTSYHMHWVRQAPGQGLEWIGYIYPGNVNTEYNEKFKGKATLTADKSTNTAYMELSSLRSEDTAVYFCAREEITYAMDYWGQGTLVTVSSGGGGSGGGGSGGGGSGGDVQMTQSPSTLSASVGDRVTITCSSSQSIVHSNGNTYMEWYQQKPGKAPKLLIYKVSNRFSGVPDRFSGSGSGTEFTLTISSLQPDDFATYYCHQGSHVPRTFGQGTKVEVK

SEQ ID NO: 212 - protein, 807 aa, synthetic construct
  REGION 1..807: note = HLA-A:02 scFv
ATGGACATGAGGGTCCCCGCTCAGCTCCTGGGGCTCCTGCTACTCTGGCTCCGAGGTGCCAGATGTCAGGTGCAGCTGGTGCAGTCTGGGGCTGAGGTGAAGAAGCCTGGGTCCTCAGTGAAGGTTTCCTGCAAGGCTTCTGGATACACCTTCACTAGCTATCATATGCATTGGGTGCGCCAGGCCCCCGGACAAGGGCTTGAGTGGATCGGATACATCTACCCTGGCAATGTTAACACAGAATATAATGAGAAGTTCAAGGGCAAAGCCACCCTTACCGCGGACAAATCCACGAACACAGCCTACATGGAGCTGAGCAGCCTGAGATCTGAAGACACGGCTGTGTATTTCTGTGCGAGGGAGGAAATTACCTACGCTATGGACTACTGGGGCCAGGGAACCCTGGTCACCGTGTCCTCAGGCGGAGGTGGAAGCGGAGGGGGAGGATCTGGCGGCGGAGGAAGCGGAGGCGACGTTCAAATGACCCAGAGCCCATCCACCCTGAGCGCATCTGTAGGTGACCGGGTCACCATCACTTGTAGCTCCAGTCAGAGTATTGTACACAGTAATGGGAACACCTATATGGAATGGTATCAGCAGAAACCAGGTAAAGCCCCAAAATTGCTCATCTACAAAGTCTCTAACAGATTTAGTGGTGTACCAGACAGGTTCAGCGGTTCCGGAAGTGGTACTGAATTCACCCTCACGATCTCCTCTCTCCAGCCAGATGATTTCGCCACTTATTACTGTCATCAAGGTTCACATGTGCCGCGCACATTCGGTCAGGGTACTAAAGTAGAAGTCAAA

SEQ ID NO: 213-218 - skipped (6 entries)

SEQ ID NO: 219 - protein, 45 aa, synthetic construct
  REGION 1..45: note = CD8alpha hinge
TTTPAPRPPTPAPTIASQPLSLRPEACRPAAGGAVHTRGLDFACD

SEQ ID NO: 220 - DNA, 135 bp, synthetic construct
  misc_feature 1..135: note = CD8alpha hinge
  source 1..135: mol_type = other DNA
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 221 - protein, 41 aa, synthetic construct
  REGION 1..41: note = CD28 hinge
CTIEVMYPPPYLDNEKSNGTIIHVKGKHLCPSPLFPGPSKP

SEQ ID NO: 222 - DNA, 123 bp, synthetic construct
  misc_feature 1..123: note = CD28 hinge
  source 1..123: mol_type = other DNA
tgtaccattgaagttatgtatcctcctccttacctagacaatgagaagagcaatggaaccattatccatgtgaaagggaaacacctttgtccaagtcccctatttcccggaccttctaagccc

SEQ ID NO: 223 - DNA, 729 bp, synthetic construct
  misc_feature 1..729: note = HLA-A11 scFv
  source 1..729: mol_type = other DNA
caggtgcagctgcaggaaagcggccctggcctggtgaaacccagccagaccctgagcctgacctgcacagtgtccggcggctcgatcagcagcggcggctactactggtcctggatcagacagccccctggcaagggcctggaatggatcggctacatctactacagcggcagcacctactacaaccccagcctgaagtccagagtgaccatcagcgtggacaccagcaagaaccagttcagcctgaagctgagcagcgtgacagccgccgacaccgctgtgtattactgtgcgagacactactactactactccatggacgtctggggcaaagggaccacggtcaccgtgtcctcaggcggaggtggaagcggagggggaggatctggcggcggaggaagcggaggcgacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctcactttcggcggcggaacaaaggtggagatcaag

SEQ ID NO: 224 - DNA, 732 bp, synthetic construct
  misc_feature 1..732: note = HLA-A11 scFv
  source 1..732: mol_type = other DNA
cagatcaccctgaaagagtccggccccaccctggtgaaacccacccagaccctgaccctgacatgcaccttcagcggcttcagcctgagcacctctggcgtgggcgtgggctggatcagacagcctcccggcaaggccctggaatggctggccctgatctactggaacgacgacaagcggtacagccccagcctgaagtcccggctgaccatcaccaaggacacctcgaagaaccaggtggtgctgaccatgacaaacatggaccccgtggacaccgccacatattactgtgcacacagacacatgcgtttaagctgttttgactactggggccagggaaccctggtcaccgtgtcctcaggcggaggtggaagcggagggggaggatctggcggcggaggaagcggaggcgacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctcactttcggcggcggaacaaaggtggagatcaag

SEQ ID NO: 225 - DNA, 735 bp, synthetic construct
  misc_feature 1..735: note = HLA-A11 scFv
  source 1..735: mol_type = other DNA
caggtgcagctggtgcagtctggcgccgaagtgaagaaacctggcgcctccgtgaaggtgtcctgcaaggccagcggctacaccttcaccagctacgccatgcactgggttcgacaggcccctggccagagactggaatggatgggctggatcaacgccggcaacggcaacaccaagtacagccagaaattccagggcagagtgaccatcacccgggacaccagcgccagcaccgcctacatggaactgagcagcctgcggagcgaggacaccgctgtgtattactgtgcgagagaaggaaatggtgccaaccctgatgcttttgatatctggggccaagggacaatggtcaccgtgtcctcaggcggaggtggaagcggagggggaggatctggcggcggaggaagcggaggcgacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctcactttcggcggcggaacaaaggtggagatcaag

SEQ ID NO: 226 - DNA, 729 bp, synthetic construct
  misc_feature 1..729: note = HLA-A11 scFv
  source 1..729: mol_type = other DNA
gaagtgcagctggtggaaagcggcggaggcctggtgcagcctggcggcagcctgagactgtcttgcgccgccagcggcttcaccttcagcagctacgacatgcactgggtccgccaggccaccggcaagggactggaatgggtgtccgccatcggcacagccggcgacacttactaccccggcagcgtgaagggccggttcaccatcagcagagagaacgccaagaacagcctgtacctgcagatgaacagccttcgagccggcgataccgccgtgtattactgtgcaagagatctccctggtagctactggtacttcgatctctggggccgtggcaccctggtcactgtgtcctcaggcggaggtggaagcggagggggaggatctggcggcggaggaagcggaggcgacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctcactttcggcggcggaacaaaggtggagatcaag

SEQ ID NO: 227 - DNA, 729 bp, synthetic construct
  misc_feature 1..729: note = HLA-A11 scFv
  source 1..729: mol_type = other DNA
caggtgcagctgcaggaaagcggccctggcctggtgaaacccagccagaccctgagcctgacctgcacagtgtccggcggctcgatcagcagcggcggctactactggtcctggatcagacagccccctggcaagggcctggaatggatcggctacatctactacagcggcagcacctactacaaccccagcctgaagtccagagtgaccatcagcgtggacaccagcaagaaccagttcagcctgaagctgagcagcgtgacagccgccgacaccgctgtgtattactgtgcgagacactactactactactacctggacgtctggggcaaagggaccacggtcaccgtgtcctcaggcggaggtggaagcggagggggaggatctggcggcggaggaagcggaggcgacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctcactttcggcggcggaacaaaggtggagatcaag

SEQ ID NO: 228 - DNA, 729 bp, synthetic construct
  misc_feature 1..729: note = HLA-A11 scFv
  source 1..729: mol_type = other DNA
gaagtgcagctggtggaaagcggcggaggcctggtgcagcctggcggcagcctgagactgtcttgcgccgccagcggcttcaccttcagcagctactggatgcactgggtccgccaggcccctggcaagggactggtctgggtgtctcgaatcaacagcgacggcagcagcaccagctacgccgacagcgtgaagggccggttcaccatcagccgggacaacgccaagaacaccctgtacctgcagatgaacagcctgcgggccgaggacaccgccgtgtattactgttgtttgggtgttttattatacaactggttcgacccctggggccagggaaccctggtcaccgtgtcctcaggcggaggtggaagcggagggggaggatctggcggcggaggaagcggaggcgacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctcactttcggcggcggaacaaaggtggagatcaag

SEQ ID NO: 229 - DNA, 726 bp, synthetic construct
  misc_feature 1..726: note = HLA-A11 scFv
  source 1..726: mol_type = other DNA
caggtgcagctgcaggaaagcggccctggcctggtgaaacccagccagaccctgagcctgacctgcacagtgtccggcggctcgatcagcagcggcggctactactggtcctggatcagacagccccctggcaagggcctggaatggatcggctacatctactacagcggcagcacctactacaaccccagcctgaagtccagagtgaccatcagcgtggacaccagcaagaaccagttcagcctgaagctgagcagcgtgacagccgccgacaccgctgtgtattactgtgcgagacactactactactacatggacgtctggggcaaagggaccacggtcaccgtgtcctcaggcggaggtggaagcggagggggaggatctggcggcggaggaagcggaggcgacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctcactttcggcggcggaacaaaggtggagatcaag

SEQ ID NO: 230 - DNA, 729 bp, synthetic construct
  misc_feature 1..729: note = HLA-A11 scFv
  source 1..729: mol_type = other DNA
cagatcaccctgaaagagtccggccccaccctggtgaaacccacccagaccctgaccctgacatgcaccttcagcggcttcagcctgagcacctctggcgtgggcgtgggctggatcagacagcctcccggcaaggccctggaatggctggccctgatctactggaacgacgacaagcggtacagccccagcctgaagtcccggctgaccatcaccaaggacacctcgaagaaccaggtggtgctgaccatgacaaacatggaccccgtggacaccgccacatattactgtgcacacaaaacgacgtcgttttactttgactactggggccagggaaccctggtcaccgtgtcctcaggcggaggtggaagcggagggggaggatctggcggcggaggaagcggaggcgacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctcactttcggcggcggaacaaaggtggagatcaag

SEQ ID NO: 231 - DNA, 729 bp, synthetic construct
  misc_feature 1..729: note = HLA-A11 scFv
  source 1..729: mol_type = other DNA
caggtgcagctgcaggaaagcggccctggcctggtgaaacccagccagaccctgagcctgacctgcacagtgtccggcggctcgatcagcagcggcggctactactggtcctggatcagacagccccctggcaagggcctggaatggatcggctacatctactacagcggcagcacctactacaaccccagcctgaagtccagagtgaccatcagcgtggacaccagcaagaaccagttcagcctgaagctgagcagcgtgacagccgccgacaccgctgtgtattactgtgcgagacactactactactactacatggacgtctggggcaaagggaccacggtcaccgtgtcctcaggcggaggtggaagcggagggggaggatctggcggcggaggaagcggaggcgacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctcactttcggcggcggaacaaaggtggagatcaag

SEQ ID NO: 232 - protein, 27 aa, synthetic construct
  REGION 1..27: note = CD28 transmembrane
FWVLVVVGGVLACYSLLVTVAFIIFWV

SEQ ID NO: 233 - DNA, 81 bp, synthetic construct
  misc_feature 1..81: note = CD28 transmembrane
  source 1..81: mol_type = other DNA
ttctgggtgctggtcgttgtgggcggcgtgctggcctgctacagcctgctggtgacagtggccttcatcatcttttgggtg

SEQ ID NO: 234 - protein, 25 aa, synthetic construct
  REGION 1..25: note = IL-2Rbeta transmembrane domain
IPWLGHLLVGLSGAFGFIILVYLLI

SEQ ID NO: 235 - DNA, 75 bp, synthetic construct
  misc_feature 1..75: note = IL-2Rbeta transmembrane domain
  source 1..75: mol_type = other DNA
attccgtggctcggccacctcctcgtgggcctcagcggggcttttggcttcatcatcttagtgtacttgctgatc

SEQ ID NO: 236 - protein, 21 aa, synthetic construct
  REGION 1..21: note = CD8 transmembrane domain
IYIWAPLAGTCGVLLLSLVIT

SEQ ID NO: 237 - DNA, 63 bp, synthetic construct
  misc_feature 1..63: note = CD8 transmembrane domain
  source 1..63: mol_type = other DNA
atctacatctgggcgcccttggccgggacttgtggggtccttctcctgtcactggttatcacc

SEQ ID NO: 238 - protein, 112 aa, synthetic construct
  REGION 1..112: note = CD3zeta activation domain
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 239 - DNA, 336 bp, synthetic construct
  misc_feature 1..336: note = CD3zeta activation domain
  source 1..336: mol_type = other DNA
agagtgaagttcagcaggagcgcagacgcccccgcgtacaagcagggccagaaccagctctataacgagctcaatctaggacgaagagaggagtacgatgttttggacaagcgtagaggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggactcagtacagccaccaaggacacctacgacgcccttcacatgcaggccctgccccctcgc

SEQ ID NO: 240 - skipped

SEQ ID NO: 241 - protein, 43 aa, synthetic construct
  REGION 1..43: note = CD3zeta activation domain
RVKFSRSADAPAYQQGQNQLYNELNLGRREEYDVLHMQALPPR

SEQ ID NO: 242 - DNA, 129 bp, synthetic construct
  misc_feature 1..129: note = CD3zeta activation domain
  source 1..129: mol_type = other DNA
agagtgaagttcagcaggagcgcagacgcccccgcgtaccagcagggccagaaccagctctataacgagctcaatctaggacgaagagaggagtacgatgttttgcacatgcaggccctgccccctcgc

SEQ ID NO: 243 - protein, 41 aa, synthetic construct
  REGION 1..41: note = CD28 costimulatory domain
RSKRSRLLHSDYMNMTPRRPGPTRKHYQPYAPPRDFAAYRS

SEQ ID NO: 244 - DNA, 123 bp, synthetic construct
  misc_feature 1..123: note = CD28 costimulatory domain
  source 1..123: mol_type = other DNA
aggagcaagcggagcagactgctgcacagcgactacatgaacatgaccccccggaggcctggccccacccggaagcactaccagccctacgcccctcccagggatttcgccgcctaccggagc

SEQ ID NO: 245 - protein, 94 aa, synthetic construct
  REGION 1..94: note = IL-2-Rbeta intracellular domain
NCRNTGPWLKKVLKCNTPDPSKFFSQLSSEHGGDVQKWLSSPFPSSSFSPGGLAPEISPLEVLERDKVTQLLPLNTDAYLSLQELQGQDPTHLV

SEQ ID NO: 246 - DNA, 282 bp, synthetic construct
  misc_feature 1..282: note = IL-2-Rbeta intracellular domain
  source 1..282: mol_type = other DNA
aactgcaggaacaccgggccatggctgaagaaggtcctgaagtgtaacaccccagacccctcgaagttcttttcccagctgagctcagagcatggaggcgacgtccagaagtggctctcttcgcccttcccctcatcgtccttcagccctggcggcctggcacctgagatctcgccactagaagtgctggagagggacaaggtgacgcagctgctccccctgaacactgatgcctacttgtctctccaagaactccagggtcaggacccaactcacttggtg

SEQ ID NO: 247 - protein, 4 aa, synthetic construct
  REGION 1..4: note = STAT5-recruitment motif(s)
YLSL

SEQ ID NO: 248 - protein, 200 aa, synthetic construct
  REGION 1..200: note = CD28-4-1BB-CD3z
RSKRSRLLHSDYMNMTPRRPGPTRKHYQPYAPPRDFAAYRSRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 249 - protein, 603 aa, synthetic construct
  REGION 1..603: note = CD28-4-1BB-CD3z
AGGAGTAAGAGGAGCAGGCTCCTGCACAGTGACTACATGAACATGACTCCCCGCCGCCCCGGGCCCACCCGCAAGCATTACCAGCCCTATGCCCCACCACGCGACTTCGCAGCCTATCGCTCCCGTTTCTCTGTTGTTAAACGGGGCAGAAAGAAGCTCCTGTATATATTCAAACAACCATTTATGAGACCAGTACAAACTACTCAAGAGGAAGATGGCTGTAGCTGCCGATTTCCAGAAGAAGAAGAAGGAGGATGTGAACTGAGAGTGAAGTTCAGCAGGAGCGCAGACGCCCCCGCGTACCAGCAGGGCCAGAACCAGCTCTATAACGAGCTCAATCTAGGACGAAGAGAGGAGTACGATGTTTTGGACAAGAGACGTGGCCGGGACCCTGAGATGGGGGGAAAGCCGAGAAGGAAGAACCCTCAGGAAGGCCTGTACAATGAACTGCAGAAAGATAAGATGGCGGAGGCCTACAGTGAGATTGGGATGAAAGGCGAGCGCCGGAGGGGCAAGGGGCACGATGGCCTTTACCAGGGTCTCAGTACAGCCACCAAGGACACCTACGACGCCCTTCACATGCAGGCCCTGCCCCCTCGCTAG

SEQ ID NO: 250 - protein, 153 aa, synthetic construct
  REGION 1..153: note = CD28-CD3z
RSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 251 - protein, 462 aa, synthetic construct
  REGION 1..462: note = CD28-CD3z
AGGAGCAAGCGGAGCAGACTGCTGCACAGCGACTACATGAACATGACCCCCCGGAGGCCTGGCCCCACCCGGAAGCACTACCAGCCCTACGCCCCTCCCAGGGATTTCGCCGCCTACCGGAGCAGAGTGAAGTTCAGCAGGAGCGCAGACGCCCCCGCGTACAAGCAGGGCCAGAACCAGCTCTATAACGAGCTCAATCTAGGACGAAGAGAGGAGTACGATGTTTTGGACAAGCGTAGAGGCCGGGACCCTGAGATGGGGGGAAAGCCGAGAAGGAAGAACCCTCAGGAAGGCCTGTACAATGAACTGCAGAAAGATAAGATGGCGGAGGCCTACAGTGAGATTGGGATGAAAGGCGAGCGCCGGAGGGGCAAGGGGCACGATGGCCTTTACCAGGGACTCAGTACAGCCACCAAGGACACCTACGACGCCCTTCACATGCAGGCCCTGCCCCCTCGCTAG

SEQ ID NO: 252 - protein, 157 aa, synthetic construct
  REGION 1..157: note = 4-1BB-CD3z
KRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 253 - DNA, 474 bp, synthetic construct
  misc_feature 1..474: note = 4-1BB-CD3z
  source 1..474: mol_type = other DNA
aaacggggcagaaagaagctcctgtatatattcaaacaaccatttatgagaccagtacaaactactcaagaggaagatggctgtagctgccgatttccagaagaagaagaaggaggatgtgaactgggcggtggcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctag

SEQ ID NO: 254 - protein, 253 aa, synthetic construct
  REGION 1..253: note = Inhibitory Receptor Hinge, Transmembrane and Intracellular Domains
YGSQSSKPYLLTHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 255 - protein, 762 aa, synthetic construct
  REGION 1..762: note = Inhibitory Receptor Hinge, Transmembrane and Intracellular Domains
TACGGCTCACAGAGCTCCAAACCCTACCTGCTGACTCACCCCAGTGACCCCCTGGAGCTCGTGGTCTCAGGACCGTCTGGGGGCCCCAGCTCCCCGACAACAGGCCCCACCTCCACATCTGGCCCTGAGGACCAGCCCCTCACCCCCACCGGGTCGGATCCCCAGAGTGGTCTGGGAAGGCACCTGGGGGTTGTGATCGGCATCTTGGTGGCCGTCATCCTACTGCTCCTCCTCCTCCTCCTCCTCTTCCTCATCCTCCGACATCGACGTCAGGGCAAACACTGGACATCGACCCAGAGAAAGGCTGATTTCCAACATCCTGCAGGGGCTGTGGGGCCAGAGCCCACAGACAGAGGCCTGCAGTGGAGGTCCAGCCCAGCTGCCGATGCCCAGGAAGAAAACCTCTATGCTGCCGTGAAGCACACACAGCCTGAGGATGGGGTGGAGATGGACACTCGGAGCCCACACGATGAAGACCCCCAGGCAGTGACGTATGCCGAGGTGAAACACTCCAGACCTAGGAGAGAAATGGCCTCTCCTCCTTCCCCACTGTCTGGGGAATTCCTGGACACAAAGGACAGACAGGCGGAAGAGGACAGGCAGATGGACACTGAGGCTGCTGCATCTGAAGCCCCCCAGGATGTGACCTACGCCCAGCTGCACAGCTTGACCCTCAGACGGGAGGCAACTGAGCCTCCTCCATCCCAGGAAGGGCCCTCTCCAGCTGTGCCCAGCATCTACGCCACTCTGGCCATCCACTAG

SEQ ID NO: 256 - protein, 6 aa, synthetic construct
  REGION 1..6: note = ITIM
NLYAAV

SEQ ID NO: 257 - protein, 6 aa, synthetic construct
  REGION 1..6: note = ITIM
VTYAEV

SEQ ID NO: 258 - protein, 6 aa, synthetic construct
  REGION 1..6: note = ITIM
VTYAQL

SEQ ID NO: 259 - protein, 6 aa, synthetic construct
  REGION 1..6: note = ITIM
SIYATL

SEQ ID NO: 260 - skipped

SEQ ID NO: 261 - protein, 58 aa, synthetic construct
  REGION 1..58: note = LILRB1 domain
VTYAEVKHSRPRREMASPPSPLSGEFLDTKDRQAEEDRQMDTEAAASEAPQDVTYAQL

SEQ ID NO: 262 - protein, 36 aa, synthetic construct
  REGION 1..36: note = LILRB1 domain
VTYAQLHSLTLRREATEPPPSQEGPSPAVPSIYATL

SEQ ID NO: 263 - protein, 87 aa, synthetic construct
  REGION 1..87: note = LILRB1 domain
NLYAAVKHTQPEDGVEMDTRSPHDEDPQAVTYAEVKHSRPRREMASPPSPLSGEFLDTKDRQAEEDRQMDTEAAASEAPQDVTYAQL

SEQ ID NO: 264 - protein, 88 aa, synthetic construct
  REGION 1..88: note = LILRB1 domain
VTYAEVKHSRPRREMASPPSPLSGEFLDTKDRQAEEDRQMDTEAAASEAPQDVTYAQLHSLTLRREATEPPPSQEGPSPAVPSIYATL

SEQ ID NO: 265 - protein, 117 aa, synthetic construct
  REGION 1..117: note = LILRB1 domain
NLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATL

SEQ ID NO: 266 - protein, 167 aa, synthetic construct
  REGION 1..167: note = LILRB1 domain
RHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 267 - protein, 10 aa, synthetic construct
  REGION 1..10: note = TCR beta intracellular domain
MAMVKRKDSR

SEQ ID NO: 268 - DNA, 30 bp, synthetic construct
  misc_feature 1..30: note = TCR beta intracellular domain
  source 1..30: mol_type = other DNA
atggccatggtcaagagaaaggattccaga

SEQ ID NO: 269 - protein, 23 aa, synthetic construct
  REGION 1..23: note = LILRB1 transmembrane domain
VVIGILVAVILLLLLLLLLFLIL

SEQ ID NO: 270 - protein, 23 aa, synthetic construct
  REGION 1..23: note = TCR alpha transmembrane
VIGFRILLLKVAGFNLLMTLRLW

SEQ ID NO: 271 - DNA, 69 bp, synthetic construct
  misc_feature 1..69: note = TCR alpha transmembrane
  source 1..69: mol_type = other DNA
gtgattgggttccgaatcctcctcctgaaagtggccgggtttaatctgctcatgacgctgcggctgtgg

SEQ ID NO: 272 - protein, 23 aa, synthetic construct
  REGION 1..23: note = TCR beta transmembrane
TILYEILLGKATLYAVLVSALVL

SEQ ID NO: 273 - DNA, 69 bp, synthetic construct
  misc_feature 1..69: note = TCR beta transmembrane
  source 1..69: mol_type = other DNA
accatcctctatgagatcttgctagggaaggccaccttgtatgccgtgctggtcagtgccctcgtgctg

SEQ ID NO: 274 - protein, 20 aa, synthetic construct
  REGION 1..20: note = LILRB1 hinge
YGSQSSKPYLLTHPSDPLEL

SEQ ID NO: 275 - protein, 63 aa, synthetic construct
  REGION 1..63: note = LILRB1 hinge
YGSQSSKPYLLTHPSDPLELVVSGPSGGPSSPTTGPTSTSGPEDQPLTPTGSDPQSGLGRHLG

SEQ ID NO: 276 - protein, 43 aa, synthetic construct
  REGION 1..43: note = LILRB1 hinge
VVSGPSGGPSSPTTGPTSTSGPEDQPLTPTGSDPQSGLGRHLG

SEQ ID NO: 277 - protein, 86 aa, synthetic construct
  REGION 1..86: note = LILRB1 hinge transmembrane
YGSQSSKPYLLTHPSDPLELVVSGPSGGPSSPTTGPTSTSGPEDQPLTPTGSDPQSGLGRHLGVVIGILVAVILLLLLLLLLFLIL

SEQ ID NO: 278 - protein, 190 aa, synthetic construct
  REGION 1..190: note = LILRB1 transmembrane and intracellular domain
VVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 279 - protein, 233 aa, synthetic construct
  REGION 1..233: note = LILRB1 fragment
VVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 280 - protein, 670 aa, Homo sapiens
MTPILTVLICLGLSLGPRTHVQAGHLPKPTLWAEPGSVITQGSPVTLRCQGGQETQEYRLYREKKTALWITRIPQELVKKGQFPIPSITWEHAGRYRCYYGSDTAGRSESSDPLELVVTGAYIKPTLSAQPSPVVNSGGNVILQCDSQVAFDGFSLCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIHPSQEGPSPAVPSIYATLAIH

SEQ ID NO: 281 - protein, 35 aa, synthetic construct
  REGION 1..35: note = ITIM1-2
NLYAAVKHTQPEDGVEMDTRSPHDEDPQAVTYAEV

SEQ ID NO: 282 - protein, 64 aa, synthetic construct
  REGION 1..64: note = hinge
YGSQSSKPYLLTHPSDPLELVVSGPSGGPSSPTTGPTSTSGPEDQPLTPTGSDPQSGLGRHLGV

SEQ ID NO: 283 - protein, 44 aa, synthetic construct
  REGION 1..44: note = Short hinge 2
VVSGPSGGPSSPTTGPTSTSGPEDQPLTPTGSDPQSGLGRHLGV

SEQ ID NO: 284 - protein, 67 aa, synthetic construct
  REGION 1..67: note = Long hinge 1
AGSGGSGGSGGSPVPSTPPTPSPSTPPTPSPSGGSGNSSGSGGSPVPSTPPTPSPSTPPTPSPSASV

SEQ ID NO: 285 - protein, 75 aa, synthetic construct
  REGION 1..75: note = Long hinge 2
AGSGGSGGSGGSPVPSTPPTNSSSTPPTPSPSPVPSTPPTNSSSTPPTPSPSPVPSTPPTNSSSTPPTPSPSASV

SEQ ID NO: 286 - protein, 85 aa, synthetic construct
  REGION 1..85: note = 2X short hinge
VVSGPSGGPSSPTTGPTSTSGPEDQPLTPTGSDPQSGLGRHVVSGPSGGPSSPTTGPTSTSGPEDQPLTPTGSDPQSGLGRHLGV

SEQ ID NO: 287 - protein, 32 aa, synthetic construct
  REGION 1..32: note = Hinge (truncated)
TTGPTSTSGPEDQPLTPTGSDPQSGLGRHLGV

SEQ ID NO: 288 - protein, 18 aa, synthetic construct
  REGION 1..18: note = T2A self-cleaving peptide
EGRGSLLTCGDVEENPGP

SEQ ID NO: 289 - protein, 20 aa, synthetic construct
  REGION 1..20: note = E2A self-cleaving peptide
QCTNYALLKLAGDVESNPGP

SEQ ID NO: 290 - protein, 22 aa, synthetic construct
  REGION 1..22: note = F2A self-cleaving peptide
VKQTLNFDLLKLAGDVESNPGP

SEQ ID NO: 291 - protein, 358 aa, Homo sapiens [sequence omitted]

SEQ ID NO: 292 - protein, 358 aa, Homo sapiens
MVDGTLLLLLSEALALTQTWAGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 293 - protein, 17 aa, synthetic construct
  REGION 1..17: note = linker
GGGGSGGGGSGGGGSGG

SEQ ID NO: 294 - protein, 22 aa, synthetic construct
  REGION 1..22: note = signal sequence
MDMRVPAQLLGLLLLWLRGARC

SEQ ID NO: 295 - DNA, 66 bp, synthetic construct
  misc_feature 1..66: note = signal sequence
  source 1..66: mol_type = other DNA
atggacatgagggtccccgctcagctcctggggctcctgctactctggctccgaggtgccagatgt

SEQ ID NO: 296 - protein, 244 aa, synthetic construct
  REGION 1..244: note = HLA-B07 scFv
QVQLQESGPGLVKPSQTLSLTCTVSGYSITSGYSWHWIRQPPGKGLEWIGYIHFSGSTHYHPSLKSRVTISVDTSKNQFSLKLSSVTAADTAVYYCARGGVVSHYAMDCWGQGTTVTVSSGGGGSGGGGSGGGGSGGDIQMTQSPSSLSASVGDRVTITCRASENIYSNLAWYQQKPGKAPKLLIYAATYLPDGVPSRFSGSGSGTDFTLTISSLQPEDFATYYCQHFWVTPYTFGGGTKVEIK

SEQ ID NO: 297 - protein, 244 aa, synthetic construct
  REGION 1..244: note = HLA-B07 scFv
EVQLVESGGGLVKPGGSLRLSCAASGYSITSGYSWHWVRQAPGKGLEWVSYIHFSGSTHYHPSLKSRFTISRDNAKNSLYLQMNSLRAEDTAVYYCARGGVVSHYAMDCWGQGTTVTVSSGGGGSGGGGSGGGGSGGDIQMTQSPSSVSASVGDRVTITCRASENIYSNLAWYQQKPGKAPKLLIYAATYLPDGVPSRFSGSGSGTDFTLTISSLQPEDFATYYCQHFWVTPYTFGGGTKVEIK

SEQ ID NO: 298 - protein, 244 aa, synthetic construct
  REGION 1..244: note = HLA-B07 scFv
EVQLVESGGGLVKPGGSLRLSCAASGYSITSGYSWHWVRQAPGKGLEWVGYIHFSGSTHYHPSLKSRFTISRDDSKNTLYLQMNSLKTEDTAVYYCARGGVVSHYAMDCWGQGTTVTVSSGGGGSGGGGSGGGGSGGEIVLTQSPATLSLSPGERATLSCRASENIYSNLAWYQQKPGQAPRLLIYAATYLPDGIPARFSGSGSGTDFTLTISSLEPEDFAVYYCQHFWVTPYTFGGGTKVEIK

SEQ ID NO: 299 - protein, 244 aa, synthetic construct
  REGION 1..244: note = HLA-B07 scFv
QVQLQQSGPGLVKPSQTLSLTCAISGYSITSGYSWHWIRQSPSRGLEWLGYIHFSGSTHYHPSLKSRITINPDTSKNQFSLQLNSVTPEDTAVYYCARGGVVSHYAMDCWGQGTTVTVSSGGGGSGGGGSGGGGSGGEIVLTQSPATLSLSPGERATLSCRASENIYSNLAWYQQKPGQAPRLLIYAATYLPDGIPARFSGSGSGTDFTLTISRLEPEDFAVYYCQHFWVTPYTFGGGTKVEIK

SEQ ID NO: 300 - protein, 244 aa, synthetic construct
  REGION 1..244: note = HLA-B07 scFv
EVQLVESGGGLVKPGGSLRLSCAASGYSITSGYSWHWVRQAPGKGLEWVGYIHFSGSTHYHPSLKSRFTISRDDSKNTLYLQMNSLKTEDTAVYYCARGGVVSHYAMDCWGQGTTVTVSSGGGGSGGGGSGGGGSGGDIQMTQSPSSVSASVGDRVTITCRASENIYSNLAWYQQKPGKAPKLLIYAATYLPDGVPSRFSGSGSGTDFTLTISSLQPEDFATYYCQHFWVTPYTFGGGTKVEIK

SEQ ID NO: 301 - protein, 244 aa, synthetic construct
  REGION 1..244: note = HLA-B07 scFv
EVQLVESGGGLVQPGGSLRLSCAASGYSITSGYSWHWVRQAPGKGLEWVSYIHFSGSTHYHPSLKSRFTISRDNSKNTLYLQMNSLRAEDTAVYYCARGGVVSHYAMDCWGQGTTVTVSSGGGGSGGGGSGGGGSGGDIQMTQSPSSLSASVGDRVTITCRASENIYSNLAWYQQKPGKAPKLLIYAATYLPDGVPSRFSGSGSGTDFTLTISSLQPEDFATYYCQHFWVTPYTFGGGTKVEIK

SEQ ID NO: 302 - protein, 244 aa, synthetic construct
  REGION 1..244: note = HLA-B07 scFv
EVQLVESGGGLVKPGGSLRLSCAASGYSITSGYSWHWVRQAPGKGLEWVGYIHFSGSTHYHPSLKSRFTISRDDSKNTLYLQMNSLKTEDTAVYYCARGGVVSHYAMDCWGQGTTVTVSSGGGGSGGGGSGGGGSGGDIQMTQSPSSLSASVGDRVTITCRASENIYSNLAWYQQKPGKAPKLLIYAATYLPDGVPSRFSGSGSGTDFTLTISSLQPEDFATYYCQHFWVTPYTFGGGTKVEIK

SEQ ID NO: 303 - protein, 244 aa, synthetic construct
  REGION 1..244: note = HLA-B07 scFv
QVQLQQWGAGLLKPSETLSLTCAVYGYSITSGYSWHWIRQPPGKGLEWIGYIHFSGSTHYHPSLKSRVTISVDTSKNQFSLKLSSVTAADTAVYYCARGGVVSHYAMDCWGQGTTVTVSSGGGGSGGGGSGGGGSGGDIQMTQSPSSLSASVGDRVTITCRASENIYSNLAWYQQKPGKAPKLLIYAATYLPDGVPSRFSGSGSGTDFTLTISSLQPEDFATYYCQHFWVTPYTFGGGTKVEIK

SEQ ID NO: 304 - protein, 244 aa, synthetic construct
  REGION 1..244: note = HLA-B07 scFv
QVQLQESGPGLVKPSQTLSLTCTVSGYSITSGYSWHWIRQHPGKGLEWIGYIHFSGSTHYHPSLKSRVTISVDTSKNQFSLKLSSVTAADTAVYYCARGGVVSHYAMDCWGQGTTVTVSSGGGGSGGGGSGGGGSGGDIQMTQSPSSLSASVGDRVTITCRASENIYSNLAWYQQKPGKAPKLLIYAATYLPDGVPSRFSGSGSGTDFTLTISSLQPEDFATYYCQHFWVTPYTFGGGTKVEIK

SEQ ID NO: 305 - protein, 244 aa, synthetic construct
  REGION 1..244: note = HLA-B07 scFv
QVQLQQSGPGLVKPSQTLSLTCAISGYSITSGYSWHWIRQSPSRGLEWLGYIHFSGSTHYHPSLKSRITINPDTSKNQFSLQLNSVTPEDTAVYYCARGGVVSHYAMDCWGQGTTVTVSSGGGGSGGGGSGGGGSGGDIQMTQSPSSLSASVGDRVTITCRASENIYSNLAWYQQKPGKAPKLLIYAATYLPDGVPSRFSGSGSGTDFTLTISSLQPEDFATYYCQHFWVTPYTFGGGTKVEIK

SEQ ID NO: 306 - protein, 240 aa, synthetic construct
  REGION 1..240: note = HLA-C07 scFv
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAVSFDWFDPWGQGTLVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 307 - protein, 248 aa, synthetic construct
  REGION 1..248: note = HLA-C07 scFv
QVQLQESGPGLVKPSQTLSLTCTVSGGSISSGGYYWSWIRQPPGKGLEWIGYIYYSGSTYYNPSLKSRVTISVDTSKNQFSLKLSSVTAADTAVYYCARERSISPYYYYYMDVWGKGTTVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 308 - protein, 247 aa, synthetic construct
  REGION 1..247: note = HLA-C07 scFv
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARDSVIWYWFDPWGQGTLVTVSSGGGGSGGGGSGGGGSGGQSVLTQPPSASGTPGQRVTISCSGSSSNIGSNTVNWYQQLPGTAPKLLIYSNNQRPSGVPDRFSGSKSGTSASLAISGLQSEDEADYYCAAWDDSLNGWVFGGGTKLTVL

SEQ ID NO: 309 - protein, 249 aa, synthetic construct
  REGION 1..249: note = HLA-C07 scFv
QVQLQESGPGLVKPSQTLSLTCTVSGGSISSGGYYWSWIRQPPGKGLEWIGYIYYSGSTYYNPSLKSRVTISVDTSKNQFSLKLSSVTAADTAVYYCAREEILPRLSYYYYMDVWGKGTTVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 310 - protein, 245 aa, synthetic construct
  REGION 1..245: note = HLA-C07 scFv
QVQLVQSGSELKKPGASVKVSCKASGYTFTSYAMNWVRQAPGQGLEWMGWINTNTGNPTYAQGFTGRFVFSFDTSVSTAYLQICSLKAEDTAVYYCARGGRAHSSWYFDLWGRGTLVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 311 - protein, 251 aa, synthetic construct
  REGION 1..251: note = HLA-C07 scFv
QVQLQESGPGLVKPSQTLSLTCTVSGGSISSGGYYWSWIRQPPGKGLEWIGYIYYSGSTYYNPSLKSRVTISVDTSKNQFSLKLSSVTAADTAVYYCARDRIKILPRLGYYYYMDVWGKGTTVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 312 - protein, 246 aa, synthetic construct
  REGION 1..246: note = HLA-C07 scFv
QVQLQESGPGLVKPSQTLSLTCTVSGGSISSGGYYWSWIRQPPGKGLEWIGYIYYSGSTYYNPSLKSRVTISVDTSKNQFSLKLSSVTAADTAVYYCARDTVIHYYYYMDVWGKGTTVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 313 - protein, 250 aa, synthetic construct
  REGION 1..250: note = HLA-C07 scFv
QVQLQESGPGLVKPSQTLSLTCTVSGGSISSGGYYWSWIRQPPGKGLEWIGYIYYSGSTYYNPSLKSRVTISVDTSKNQFSLKLSSVTAADTAVYYCARDVIVEVFLSYYYYMDVWGKGTTVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 314 - protein, 246 aa, synthetic construct
  REGION 1..246: note = HLA-C07 scFv
QVQLQESGPGLVKPSQTLSLTCTVSGGSISSGGYYWSWIRQPPGKGLEWIGYIYYSGSTYYNPSLKSRVTISVDTSKNQFSLKLSSVTAADTAVYYCARDIFIHYYYYMDVWGKGTTVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 315 - protein, 247 aa, synthetic construct
  REGION 1..247: note = HLA-C07 scFv
EVQLVESGGGLVQPGGSLRLSCAASGFTFSSYSMNWVRQAPGKGLEWVSYISSSSSTIYYADSVKGRFTISRDNAKNSLYLQMNSLRAEDTAVYYCARDGTFYSYSPYYFDYWGQGTLVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 316 - protein, 251 aa, synthetic construct
  REGION 1..251: note = HLA-C07 scFv
QVQLQESGPGLVKPSQTLSLTCTVSGGSISSGGYYWSWIRQPPGKGLEWIGYIYYSGSTYYNPSLKSRVTISVDTSKNQFSLKLSSVTAADTAVYYCAREWIKILPRLGYYYYMDVWGKGTTVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 317 - protein, 246 aa, synthetic construct
  REGION 1..246: note = HLA-C07 scFv
QVQLQESGPGLVKPSQTLSLTCTVSGGSISSGGYYWSWIRQPPGKGLEWIGYIYYSGSTYYNPSLKSRVTISVDTSKNQFSLKLSSVTAADTAVYYCARDRSLYYYYYMDVWGKGTTVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 318 - protein, 248 aa, synthetic construct
  REGION 1..248: note = HLA-C07 scFv
QVQLQESGPGLVKPSQTLSLTCTVSGGSISSGGYYWSWIRQPPGKGLEWIGYIYYSGSTYYNPSLKSRVTISVDTSKNQFSLKLSSVTAADTAVYYCARDKILAPNYYYYMDVWGKGTTVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 319 - protein, 250 aa, synthetic construct
  REGION 1..250: note = HLA-C07 scFv
QVQLQESGPGLVKPSQTLSLTCTVSGGSISSGGYYWSWIRQPPGKGLEWIGYIYYSGSTYYNPSLKSRVTISVDTSKNQFSLKLSSVTAADTAVYYCAREKSWKYFYYYYYYMDVWGKGTTVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 320 - protein, 249 aa, synthetic construct
  REGION 1..249: note = HLA-C07 scFv
QVQLQESGPGLVKPSQTLSLTCTVSGGSISSGGYYWSWIRQPPGKGLEWIGYIYYSGSTYYNPSLKSRVTISVDTSKNQFSLKLSSVTAADTAVYYCARENTSTIPYYYYYMDVWGKGTTVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 321 - protein, 253 aa, synthetic construct
  REGION 1..253: note = HLA-C07 scFv
QVQLQESGPGLVKPSQTLSLTCTVSGGSISSGGYYWSWIRQPPGKGLEWIGYIYYSGSTYYNPSLKSRVTISVDTSKNQFSLKLSSVTAADTAVYYCAREDVDKNTSTIYYYYYYMDVWGKGTTVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 322 - protein, 250 aa, synthetic construct
  REGION 1..250: note = HLA-C07 scFv
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARDGGDIVSSSAIYWYFDLWGRGTLVTVSSGGGGSGGGGSGGGGSGGAIQLTQSPSSLSASVGDRVTITCRASQGISSALAWYQQKPGKAPKLLIYDASSLESGVPSRFSGSGSGTDFTLTISSLQPEDFATYYCQQFNSYPLTFGGGTKVEIK

SEQ ID NO: 323 - protein, 248 aa, synthetic construct
  REGION 1..248: note = HLA-C07 scFv
QVQLQESGPGLVKPSQTLSLTCTVSGGSISSGGYYWSWIRQPPGKGLEWIGYIYYSGSTYYNPSLKSRVTISVDTSKNQFSLKLSSVTAADTAVYYCARDLILPPYYYYYMDVWGKGTTVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 324 - protein, 253 aa, synthetic construct
  REGION 1..253: note = HLA-C07 scFv
QVQLQESGPGLVKPSQTLSLTCTVSGGSISSGGYYWSWIRQPPGKGLEWIGYIYYSGSTYYNPSLKSRVTISVDTSKNQFSLKLSSVTAADTAVYYCARETWIKILPRYYYYYYYMDVWGKGTTVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 325 - protein, 246 aa, synthetic construct
  REGION 1..246: note = HLA-C07 scFv
QVQLQESGPGLVKPSQTLSLTCTVSGGSISSGGYYWSWIRQPPGKGLEWIGYIYYSGSTYYNPSLKSRVTISVDTSKNQFSLKLSSVTAADTAVYYCARDLSRYYYYYMDVWGKGTTVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 326 - protein, 242 aa, synthetic construct
  REGION 1..242: note = HLA-C07 scFv
EVQLVESGGGLVQPGGSLRLSCAASGFTFSSYSMNWVRQAPGKGLEWVSYISSSSSTIYYADSVKGRFTISRDNAKNSLYLQMNSLRAEDTAVYYCAREHIVLCFDYWGQGTLVTVSSGGGGSGGGGSGGGGSGGDIQMTQSPSSLSASVGDRVTITCRASQGISSWLAWYQQKPEKAPKSLIYAASSLQSGVPSRFSGSGSGTDFTLTISSLQPEDFATYYCQQYNSYPLTFGGGTKVEIK

SEQ ID NO: 327 - protein, 249 aa, synthetic construct
  REGION 1..249: note = HLA-C07 scFv
QVQLQESGPGLVKPSQTLSLTCTVSGGSISSGGYYWSWIRQPPGKGLEWIGYIYYSGSTYYNPSLKSRVTISVDTSKNQFSLKLSSVTAADTAVYYCARDKILPRPYYYYYMDVWGKGTTVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 328 - protein, 244 aa, synthetic construct
  REGION 1..244: note = HLA-C07 scFv
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARGSNEYFQHWGQGTLVTVSSGGGGSGGGGSGGGGSGGQSALTQPPSASGSPGQSVTISCTGTSSDVGGYNYVSWYQQHPGKAPKLMIYEVSKRPSGVPDRFSGSKSGNTASLTVSGLQAEDEADYYCSSYAGSNNWVFGGGTKLTVL

SEQ ID NO: 329 - protein, 242 aa, synthetic construct
  REGION 1..242: note = HLA-C07 scFv
QVQLVQSGSELKKPGASVKVSCKASGYTFTSYAMNWVRQAPGQGLEWMGWINTNTGNPTYAQGFTGRFVFSFDTSVSTAYLQICSLKAEDTAVYYCARGTSYWYFDLWGRGTLVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 330 - protein, 248 aa, synthetic construct
  REGION 1..248: note = HLA-C07 scFv
QVQLQESGPGLVKPSQTLSLTCTVSGGSISSGGYYWSWIRQPPGKGLEWIGYIYYSGSTYYNPSLKSRVTISVDTSKNQFSLKLSSVTAADTAVYYCAREEIVEVFYYYYMDVWGKGTTVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 331 - protein, 241 aa, synthetic construct
  REGION 1..241: note = HLA-C07 scFv
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKVDDYYFDYWGQGTLVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 332 - protein, 247 aa, synthetic construct
  REGION 1..247: note = HLA-C07 scFv
EVQLVESGGGLVQPGGSLRLSCAASGFTFSSYWMHWVRQAPGKGLVWVSRINSDGSSTSYADSVKGRFTISRDNAKNTLYLQMNSLRAEDTAVYYCAWSTNILLSYTKAFDIWGQGTMVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 333 - protein, 246 aa, synthetic construct
  REGION 1..246: note = HLA-C07 scFv
QVQLQESGPGLVKPSQTLSLTCTVSGGSISSGGYYWSWIRQPPGKGLEWIGYIYYSGSTYYNPSLKSRVTISVDTSKNQFSLKLSSVTAADTAVYYCARDKTYYYYYYMDVWGKGTTVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 334 - protein, 253 aa, synthetic construct
  REGION 1..253: note = HLA-C07 scFv
QVQLQESGPGLVKPSQTLSLTCTVSGGSISSGGYYWSWIRQPPGKGLEWIGYIYYSGSTYYNPSLKSRVTISVDTSKNQFSLKLSSVTAADTAVYYCAREKYFHDKYFHDYYYYYMDVWGKGTTVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 335 - protein, 246 aa, synthetic construct
  REGION 1..246: note = HLA-C07 scFv
QVQLQESGPGLVKPSQTLSLTCTVSGGSISSGGYYWSWIRQPPGKGLEWIGYIYYSGSTYYNPSLKSRVTISVDTSKNQFSLKLSSVTAADTAVYYCARDTSVYYYYYMDVWGKGTTVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 336 - protein, 248 aa, synthetic construct
  REGION 1..248: note = HLA-C07 scFv
QVQLQESGPGLVKPSQTLSLTCTVSGGSISSGGYYWSWIRQPPGKGLEWIGYIYYSGSTYYNPSLKSRVTISVDTSKNQFSLKLSSVTAADTAVYYCAREKILPYYYYYYMDVWGKGTTVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 337 - protein, 257 aa, synthetic construct
  REGION 1..257: note = HLA-C07 scFv
EVQLVESGGGLVQPGGSLRLSCAASGFTFSSYSMNWVRQAPGKGLEWVSYISSSSSTIYYADSVKGRFTISRDNAKNSLYLQMNSLRAEDTAVYYCAIQWIYIYINPRGFIFLHDAFDIWGQGTMVTVSSGGGGSGGGGSGGGGSGGQSVLTQPPSASGTPGQRVTISCSGSSSNIGSNTVNWYQQLPGTAPKLLIYSNNQRPSGVPDRFSGSKSGTSASLAISGLQSEDEADYYCAAWDDSLNGWVFGGGTKLTVL

SEQ ID NO: 338 - protein, 248 aa, synthetic construct
  REGION 1..248: note = HLA-C07 scFv
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAKEDVDFHHDAFDIWGQGTMVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 339 - protein, 253 aa, synthetic construct
  REGION 1..253: note = HLA-C07 scFv
QVQLQESGPGLVKPSQTLSLTCTVSGGSISSGGYYWSWIRQPPGKGLEWIGYIYYSGSTYYNPSLKSRVTISVDTSKNQFSLKLSSVTAADTAVYYCAREGVDKNTSTIYYYYYYMDVWGKGTTVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 340 - protein, 241 aa, synthetic construct
  REGION 1..241: note = HLA-C07 scFv
EVQLVESGGGLVQPGGSLRLSCAASGFTFSSYSMNWVRQAPGKGLEWVSYISSSSSTIYYADSVKGRFTISRDNAKNSLYLQMNSLRAEDTAVYYCARDRRGYFDLWGRGTLVTVSSGGGGSGGGGSGGGGSGGDIQMTQSPSSLSASVGDRVTITCRASQGISSWLAWYQQKPEKAPKSLIYAASSLQSGVPSRFSGSGSGTDFTLTISSLQPEDFATYYCQQYNSYPLTFGGGTKVEIK

SEQ ID NO: 341 - protein, 248 aa, synthetic construct
  REGION 1..248: note = HLA-C07 scFv
EVQLVQSGAEVKKPGATVKISCKVSGYTFTDYYMHWVQQAPGKGLEWMGLVDPEDGETIYAEKFQGRVTITADTSTDTAYMELSSLRSEDTAVYYCATGIHVDIRSMEDWFDPWGQGTLVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 342 - protein, 246 aa, synthetic construct
  REGION 1..246: note = HLA-C07 scFv
QVQLQESGPGLVKPSQTLSLTCTVSGGSISSGGYYWSWIRQPPGKGLEWIGYIYYSGSTYYNPSLKSRVTISVDTSKNQFSLKLSSVTAADTAVYYCARDIGTSYYYYMDVWGKGTTVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 343 - protein, 249 aa, synthetic construct
  REGION 1..249: note = HLA-C07 scFv
QVQLQESGPGLVKPSQTLSLTCTVSGGSISSGGYYWSWIRQPPGKGLEWIGYIYYSGSTYYNPSLKSRVTISVDTSKNQFSLKLSSVTAADTAVYYCAREVVEVFLYYYYYMDVWGKGTTVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 344 - protein, 246 aa, synthetic construct
  REGION 1..246: note = HLA-C07 scFv
QVQLQESGPGLVKPSQTLSLTCTVSGGSISSGGYYWSWIRQPPGKGLEWIGYIYYSGSTYYNPSLKSRVTISVDTSKNQFSLKLSSVTAADTAVYYCARDLYYYYYYYMDVWGKGTTVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 345 - protein, 256 aa, synthetic construct
  REGION 1..256: note = HLA-C07 scFv
QVQLQESGPGLVKPSQTLSLTCTVSGGSISSGGYYWSWIRQPPGKGLEWIGYIYYSGSTYYNPSLKSRVTISVDTSKNQFSLKLSSVTAADTAVYYCARESWKYFYPRGSIFIHYYYYMDVWGKGTTVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 346 - protein, 248 aa, synthetic construct
  REGION 1..248: note = HLA-C07 scFv
QVQLQESGPGLVKPSQTLSLTCTVSGGSISSGGYYWSWIRQPPGKGLEWIGYIYYSGSTYYNPSLKSRVTISVDTSKNQFSLKLSSVTAADTAVYYCARDRIVEVFYYYYMDVWGKGTTVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 347 - protein, 250 aa, synthetic construct
  REGION 1..250: note = HLA-C07 scFv
QVQLQESGPGLVKPSQTLSLTCTVSGGSISSGGYYWSWIRQPPGKGLEWIGYIYYSGSTYYNPSLKSRVTISVDTSKNQFSLKLSSVTAADTAVYYCAREKYFHDWLYYYYYMDVWGKGTTVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 348 - protein, 250 aa, synthetic construct
  REGION 1..250: note = HLA-C07 scFv
QVQLQESGPGLVKPSQTLSLTCTVSGGSISSGGYYWSWIRQPPGKGLEWIGYIYYSGSTYYNPSLKSRVTISVDTSKNQFSLKLSSVTAADTAVYYCARDLVDKNTSYYYYYMDVWGKGTTVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 349 - protein, 244 aa, synthetic construct
  REGION 1..244: note = HLA-C07 scFv
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARVQNEYFQHWGQGTLVTVSSGGGGSGGGGSGGGGSGGQSALTQPPSASGSPGQSVTISCTGTSSDVGGYNYVSWYQQHPGKAPKLMIYEVSKRPSGVPDRFSGSKSGNTASLTVSGLQAEDEADYYCSSYAGSNNWVFGGGTKLTVL

SEQ ID NO: 350 - protein, 239 aa, synthetic construct
  REGION 1..239: note = HLA-C07 scFv
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCATANWFDPWGQGTLVTVSSGGGGSGGGGSGGGGSGGDIQMTQSPSSVSASVGDRVTITCRASQGISSWLAWYQQKPGKAPKLLIYAASSLQSGVPSRFSGSGSGTDFTLTISSLQPEDFATYYCQQANSFPLTFGGGTKVEIK

SEQ ID NO: 351 - protein, 244 aa, synthetic construct
  REGION 1..244: note = HLA-A03 scFv
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARERVSQRGAFDIWGQGTMVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 352 - protein, 243 aa, synthetic construct
  REGION 1..243: note = HLA-A03 scFv
EVQLVESGGGLVQPGGSLRLSCAASGFTFSSYSMNWVRQAPGKGLEWVSYISSSSSTIYYADSVKGRFTISRDNAKNSLYLQMNSLRAEDTAVYYCARGNPDKDPFDYWGQGTLVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 353 - protein, 245 aa, synthetic construct
  REGION 1..245: note = HLA-A03 scFv
QVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCARDFYCTNWYFDLWGRGTLVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 354 - protein, 244 aa, synthetic construct
  REGION 1..244: note = HLA-A03 scFv
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCARESSSGSYWYFDLWGRGTLVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 355 - protein, 250 aa, synthetic construct
  REGION 1..250: note = HLA-A03 scFv
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARDSGYKYNLYYYYYYMDVWGKGTTVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 356 - protein, 249 aa, synthetic construct
  REGION 1..249: note = HLA-A03 scFv
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARGGDLSHYYYYMDVWGKGTTVTVSSGGGGSGGGGSGGGGSGGQTVVTQEPSLTVSPGGTVTLTCASSTGAVTSGYYPNWFQQKPGQAPRALIYSTSNKHSWTPARFSGSLLGGKAALTLSGVQPEDEAEYYCLLYYGGAQWVFGGGTKLTVL

SEQ ID NO: 357 - protein, 246 aa, synthetic construct
  REGION 1..246: note = HLA-A03 scFv
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARENRRYNSCYYFDYWGQGTLVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 358 - protein, 249 aa, synthetic construct
  REGION 1..249: note = HLA-A03 scFv
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARGGDLSHYYYYLDVWGKGTTVTVSSGGGGSGGGGSGGGGSGGQTVVTQEPSLTVSPGGTVTLTCASSTGAVTSGYYPNWFQQKPGQAPRALIYSTSNKHSWTPARFSGSLLGGKAALTLSGVQPEDEAEYYCLLYYGGAQWVFGGGTKLTVL

SEQ ID NO: 359 - protein, 245 aa, synthetic construct
  REGION 1..245: note = HLA-A03 scFv
EVQLVESGGGLVQPGGSLRLSCAASGFTVSSNYMSWVRQAPGKGLEWVSVIYSGGSTYYADSVKGRFTISRDNSKNTLYLQMNSLRAEDTAVYYCARATLLSLSYDAFDIWGQGTMVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 360 - protein, 248 aa, synthetic construct
  REGION 1..248: note = HLA-A03 scFv
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARGGDLSHYYYMDVWGKGTTVTVSSGGGGSGGGGSGGGGSGGQTVVTQEPSLTVSPGGTVTLTCASSTGAVTSGYYPNWFQQKPGQAPRALIYSTSNKHSWTPARFSGSLLGGKAALTLSGVQPEDEAEYYCLLYYGGAQWVFGGGTKLTVL

SEQ ID NO: 361 - protein, 241 aa, synthetic construct
  REGION 1..241: note = HLA-A03 scFv
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARERDRWFDPWGQGTLVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 362 - protein, 247 aa, synthetic construct
  REGION 1..247: note = HLA-A03 scFv
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARETPPSLGAFDIWGQGTMVTVSSGGGGSGGGGSGGGGSGGQSALTQPPSASGSPGQSVTISCTGTSSDVGGYNYVSWYQQHPGKAPKLMIYEVSKRPSGVPDRFSGSKSGNTASLTVSGLQAEDEADYYCSSYAGSNNWVFGGGTKLTVL

SEQ ID NO: 363 - protein, 251 aa, synthetic construct
  REGION 1..251: note = HLA-A03 scFv
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCAREAYCLSDSYWYFDLWGRGTLVTVSSGGGGSGGGGSGGGGSGGQSVLTQPPSASGTPGQRVTISCSGSSSNIGSNTVNWYQQLPGTAPKLLIYSNNQRPSGVPDRFSGSKSGTSASLAISGLQSEDEADYYCAAWDDSLNGWVFGGGTKLTVL

SEQ ID NO: 364 - protein, 248 aa, synthetic construct
  REGION 1..248: note = HLA-A03 scFv
QVQLQESGPGLVKPSQTLSLTCTVSGGSISSGGYYWSWIRQPPGKGLEWIGYIYYSGSTYYNPSLKSRVTISVDTSKNQFSLKLSSVTAADTAVYYCARESWKYFYPRGYMDVWGKGTTVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 365 - protein, 248 aa, synthetic construct
  REGION 1..248: note = HLA-A01 scFv
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARGGWTAWYYYMDVWGKGTTVTVSSGGGGSGGGGSGGGGSGGQTVVTQEPSLTVSPGGTVTLTCASSTGAVTSGYYPNWFQQKPGQAPRALIYSTSNKHSWTPARFSGSLLGGKAALTLSGVQPEDEAEYYCLLYYGGAQWVFGGGTKLTVL

SEQ ID NO: 366 - protein, 244 aa, synthetic construct
  REGION 1..244: note = HLA-A01 scFv
EVQLVESGGGLVQPGGSLRLSCAASGFTFSSYSMNWVRQAPGKGLEWVSYISSSSSTIYYADSVKGRFTISRDNAKNSLYLQMNSLRAEDTAVYYCARAKYYYMDVWGKGTTVTVSSGGGGSGGGGSGGGGSGGQSVLTQPPSASGTPGQRVTISCSGSSSNIGSNTVNWYQQLPGTAPKLLIYSNNQRPSGVPDRFSGSKSGTSASLAISGLQSEDEADYYCAAWDDSLNGWVFGGGTKLTVL

SEQ ID NO: 367 - protein, 248 aa, synthetic construct
  REGION 1..248: note = HLA-A01 scFv
QVQLQESGPGLVKPSQTLSLTCTVSGGSISSGGYYWSWIRQPPGKGLEWIGYIYYSGSTYYNPSLKSRVTISVDTSKNQFSLKLSSVTAADTAVYYCARDQVDKNTYYYYMDVWGKGTTVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 368 - protein, 243 aa, synthetic construct
  REGION 1..243: note = HLA-A01 scFv
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARACQLAEYFQHWGQGTLVTVSSGGGGSGGGGSGGGGSGGDIQMTQSPSSVSASVGDRVTITCRASQGISSWLAWYQQKPGKAPKLLIYAASSLQSGVPSRFSGSGSGTDFTLTISSLQPEDFATYYCQQANSFPLTFGGGTKVEIK

SEQ ID NO: 369 - protein, 248 aa, synthetic construct
  REGION 1..248: note = HLA-A01 scFv
QVQLQESGPGLVKPSQTLSLTCTVSGGSISSGGYYWSWIRQPPGKGLEWIGYIYYSGSTYYNPSLKSRVTISVDTSKNQFSLKLSSVTAADTAVYYCARDRVDKNTSYYYMDVWGKGTTVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 370 - protein, 245 aa, synthetic construct
  REGION 1..245: note = HLA-A01 scFv
QVQLQESGPGLVKPSDTLSLTCAVSGYSISSSNWWGWIRQPPGKGLEWIGYIYYSGSTYYNPSLKSRVTMSVDTSKNQFSLKLSSVTAVDTAVYYCARRVQLKLVHWFDPWGQGTLVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 371 - protein, 245 aa, synthetic construct
  REGION 1..245: note = HLA-A01 scFv
QVQLVQSGAEVKKPGASVKVSCKASGYTFTSYDINWVRQATGQGLEWMGWMNPNSGNTGYAQKFQGRVTMTRNTSISTAYMELSSLRSEDTAVYYCATYYDYVTVFYFQHWGQGTLVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 372 - protein, 245 aa, synthetic construct
  REGION 1..245: note = HLA-A01 scFv
QLQLQESGSGLVKPSQTLSLTCAVSGGSISSGGYSWSWIRQPPGKGLEWIGYIYHSGSTYYNPSLKSRVTISVDRSKNQFSLKLSSVTAADTAVYYCARESYPSFYAFDIWGQGTMVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 373 - protein, 249 aa, synthetic construct
  REGION 1..249: note = HLA-A01 scFv
QITLKESGPTLVKPTQTLTLTCTFSGFSLSTSGVGVGWIRQPPGKALEWLALIYWNDDKRYSPSLKSRLTITKDTSKNQVVLTMTNMDPVDTATYYCAHSNMWSYSLNDYYFDYWGQGTLVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 374 - protein, 5 aa, synthetic construct
  REGION 1..5: note = CDR-H1
SYGIS

SEQ ID NO: 375 - protein, 17 aa, synthetic construct
  REGION 1..17: note = CDR-H2
WISAYNGNTNYAQKLQG

SEQ ID NO: 376 - protein, 11 aa, synthetic construct
  REGION 1..11: note = CDR-H3
ERVSQRGAFDI

SEQ ID NO: 377 - protein, 5 aa, synthetic construct
  REGION 1..5: note = CDR-H1
SYSMN

SEQ ID NO: 378 - protein, 17 aa, synthetic construct
  REGION 1..17: note = CDR-H2
YISSSSSTIYYADSVKG

SEQ ID NO: 379 - protein, 10 aa, synthetic construct
  REGION 1..10: note = CDR-H3
GNPDKDPFDY

SEQ ID NO: 380 - protein, 7 aa, synthetic construct
  REGION 1..7: note = CDR-H1
SGSYYWS

SEQ ID NO: 381 - protein, 16 aa, synthetic construct
  REGION 1..16: note = CDR-H2
YIYYSGSTNYNPSLKS

SEQ ID NO: 382 - protein, 11 aa, synthetic construct
  REGION 1..11: note = CDR-H3
DFYCTNWYFDL

SEQ ID NO: 383 - protein, 5 aa, synthetic construct
  REGION 1..5: note = CDR-H1
SYYWS

SEQ ID NO: 384 - protein, 12 aa, synthetic construct
  REGION 1..12: note = CDR-H3
ESSSGSYWYFDL

SEQ ID NO: 385 - protein, 5 aa, synthetic construct
  REGION 1..5: note = CDR-H1
SYWIG

SEQ ID NO: 386 - protein, 17 aa, synthetic construct
  REGION 1..17: note = CDR-H2
IIYPGDSDTRYSPSFQG

SEQ ID NO: 387 - protein, 17 aa, synthetic construct
  REGION 1..17: note = CDR-H3
DSGYKYNLYYYYYYMDV

SEQ ID NO: 388 - protein, 14 aa, synthetic construct
  REGION 1..14: note = CDR-L1
ASSTGAVTSGYYPN

SEQ ID NO: 389 - protein, 7 aa, synthetic construct
  REGION 1..7: note = CDR-L2
STSNKHS

SEQ ID NO: 390 - protein, 10 aa, synthetic construct
  REGION 1..10: note = CDR-L3
LLYYGGAQWV

SEQ ID NO: 391 - protein, 13 aa, synthetic construct
  REGION 1..13: note = CDR-H3
ENRRYNSCYYFDY

SEQ ID NO: 392 - protein, 13 aa, synthetic construct
  REGION 1..13: note = CDR-H3
GGDLSHYYYYLDV

SEQ ID NO: 393 - protein, 5 aa, synthetic construct
  REGION 1..5: note = CDR-H1
SNYMS

SEQ ID NO: 394 - protein, 16 aa, synthetic construct
  REGION 1..16: note = CDR-H2
VIYSGGSTYYADSVKG

SEQ ID NO: 395 - protein, 13 aa, synthetic construct
  REGION 1..13: note = CDR-H3
ATLLSLSYDAFDI

SEQ ID NO: 396 - protein, 12 aa, synthetic construct
  REGION 1..12: note = CDR-H3
GGDLSHYYYMDV

SEQ ID NO: 397 - protein, 8 aa, synthetic construct
  REGION 1..8: note = CDR-H3
ERDRWFDP

SEQ ID NO: 398 - protein, 14 aa, synthetic construct
  REGION 1..14: note = CDR-L1
TGTSSDVGGYNYVS

SEQ ID NO: 399 - protein, 7 aa, synthetic construct
  REGION 1..7: note = CDR-L2
EVSKRPS

SEQ ID NO: 400 - protein, 10 aa, synthetic construct
  REGION 1..10: note = CDR-L3
SSYAGSNNWV

SEQ ID NO: 401 - protein, 11 aa, synthetic construct
  REGION 1..11: note = CDR-H3
ETPPSLGAFDI

SEQ ID NO: 402 - protein, 13 aa, synthetic construct
  REGION 1..13: note = CDR-L1
SGSSSNIGSNTVN

SEQ ID NO: 403 - protein, 7 aa, synthetic construct
  REGION 1..7: note = CDR-L2
SNNQRPS

SEQ ID NO: 404 - protein, 11 aa, synthetic construct
  REGION 1..11: note = CDR-L3
AAWDDSLNGWV

SEQ ID NO: 405 - protein, 7 aa, synthetic construct
  REGION 1..7: note = CDR-H1
SSSYYWG

SEQ ID NO: 406 - protein, 16 aa, synthetic construct
  REGION 1..16: note = CDR-H2
SIYYSGSTYYNPSLKS

SEQ ID NO: 407 - protein, 14 aa, synthetic construct
  REGION 1..14: note = CDR-H3
EAYCLSDSYWYFDL

SEQ ID NO: 408 - protein, 14 aa, synthetic construct
  REGION 1..14: note = CDR-H3
ESWKYFYPRGYMDV

SEQ ID NO: 409 - protein, 11 aa, synthetic construct
  REGION 1..11: note = CDR-L1
RASENIYSNLA

SEQ ID NO: 410 - protein, 7 aa, synthetic construct
  REGION 1..7: note = CDR-L2
AATYLPD

SEQ ID NO: 411 - protein, 9 aa, synthetic construct
  REGION 1..9: note = CDR-L3
QHFWVTPYT

SEQ ID NO: 412 - protein, 6 aa, synthetic construct
  REGION 1..6: note = CDR-H1
SGYSWH

SEQ ID NO: 413 - protein, 16 aa, synthetic construct
  REGION 1..16: note = CDR-H2
YIHFSGSTHYHPSLKS

SEQ ID NO: 414 - protein, 11 aa, synthetic construct
  REGION 1..11: note = CDR-H3
GGVVSHYAMDC

SEQ ID NO: 415 - protein, 5 aa, synthetic construct
  REGION 1..5: note = CDR-H1
SYAMS

SEQ ID NO: 416 - protein, 17 aa, synthetic construct
  REGION 1..17: note = CDR-H2
AISGSGGSTYYADSVKG

SEQ ID NO: 417 - protein, 7 aa, synthetic construct
  REGION 1..7: note = CDR-H3
SFDWFDP

SEQ ID NO: 418 - protein, 14 aa, synthetic construct
  REGION 1..14: note = CDR-H3
ERSISPYYYYYMDV

SEQ ID NO: 419 - protein, 10 aa, synthetic construct
  REGION 1..10: note = CDR-H3
DSVIWYWFDP

SEQ ID NO: 420 - protein, 15 aa, synthetic construct
  REGION 1..15: note = CDR-H3
EEILPRLSYYYYMDV

SEQ ID NO: 421 - protein, 5 aa, synthetic construct
  REGION 1..5: note = CDR-H1
SYAMN

SEQ ID NO: 422 - protein, 17 aa, synthetic construct
  REGION 1..17: note = CDR-H2
WINTNTGNPTYAQGFTG

SEQ ID NO: 423 - protein, 12 aa, synthetic construct
  REGION 1..12: note = CDR-H3
GGRAHSSWYFDL

SEQ ID NO: 424 - protein, 17 aa, synthetic construct
  REGION 1..17: note = CDR-H3
DRIKILPRLGYYYYMDV

SEQ ID NO: 425 - protein, 12 aa, synthetic construct
  REGION 1..12: note = CDR-H3
DTVIHYYYYMDV

SEQ ID NO: 426 - protein, 16 aa, synthetic construct
  REGION 1..16: note = CDR-H3
DVIVEVFLSYYYYMDV

SEQ ID NO: 427 - protein, 12 aa, synthetic construct
  REGION 1..12: note = CDR-H3
DIFIHYYYYMDV

SEQ ID NO: 428 - protein, 14 aa, synthetic construct
  REGION 1..14: note = CDR-H3
DGTFYSYSPYYFDY

SEQ ID NO: 429 - protein, 17 aa, synthetic construct
  REGION 1..17: note = CDR-H3
EWIKILPRLGYYYYMDV

SEQ ID NO: 430 - protein, 12 aa, synthetic construct
  REGION 1..12: note = CDR-H3
DRSLYYYYYMDV

SEQ ID NO: 431 - protein, 14 aa, synthetic construct
  REGION 1..14: note = CDR-H3
DKILAPNYYYYMDV

SEQ ID NO: 432 - protein, 16 aa, synthetic construct
  REGION 1..16: note = CDR-H3
EKSWKYFYYYYYYMDV

SEQ ID NO: 433 - protein, 15 aa, synthetic construct
  REGION 1..15: note = CDR-H3
ENTSTIPYYYYYMDV

SEQ ID NO: 434 - protein, 19 aa, synthetic construct
  REGION 1..19: note = CDR-H3
EDVDKNTSTIYYYYYYMDV

SEQ ID NO: 435 - protein, 11 aa, synthetic construct
  REGION 1..11: note = CDR-L1
RASQGISSALA

SEQ ID NO: 436 - protein, 7 aa, synthetic construct
  REGION 1..7: note = CDR-L2
DASSLES

SEQ ID NO: 437 - protein, 9 aa, synthetic construct
  REGION 1..9: note = CDR-L3
QQFNSYPLT

SEQ ID NO: 438 - protein, 5 aa, synthetic construct
  REGION 1..5: note = CDR-H1
DYYMS

SEQ ID NO: 439 - protein, 17 aa, synthetic construct
  REGION 1..17: note = CDR-H2
YISSSGSTIYYADSVKG

SEQ ID NO: 440 - protein, 17 aa, synthetic construct
  REGION 1..17: note = CDR-H3
DGGDIVSSSAIYWYFDL

SEQ ID NO: 441 - protein, 14 aa, synthetic construct
  REGION 1..14: note = CDR-H3
DLILPPYYYYYMDV

SEQ ID NO: 442 - protein, 19 aa, synthetic construct
  REGION 1..19: note = CDR-H3
ETWIKILPRYYYYYYYMDV

SEQ ID NO: 443 - protein, 12 aa, synthetic construct
  REGION 1..12: note = CDR-H3
DLSRYYYYYMDV

SEQ ID NO: 444 - protein, 11 aa, synthetic construct
  REGION 1..11: note = CDR-L1
RASQGISSWLA

SEQ ID NO: 445 - protein, 9 aa, synthetic construct
  REGION 1..9: note = CDR-L3
QQYNSYPLT

SEQ ID NO: 446 - protein, 9 aa, synthetic construct
  REGION 1..9: note = CDR-H3
EHIVLCFDY

SEQ ID NO: 447 - protein, 15 aa, synthetic construct
  REGION 1..15: note = CDR-H3
DKILPRPYYYYYMDV

SEQ ID NO: 448 - protein, 8 aa, synthetic construct
  REGION 1..8: note = CDR-H3
GSNEYFQH

SEQ ID NO: 449 - protein, 9 aa, synthetic construct
  REGION 1..9: note = CDR-H3
GTSYWYFDL

SEQ ID NO: 450 - protein, 14 aa, synthetic construct
  REGION 1..14: note = CDR-H3
EEIVEVFYYYYMDV

SEQ ID NO: 451 - protein, 8 aa, synthetic construct
  REGION 1..8: note = CDR-H3
VDDYYFDY

SEQ ID NO: 452 - protein, 14 aa, synthetic construct
  REGION 1..14: note = CDR-H3
STNILLSYTKAFDI

SEQ ID NO: 453 - protein, 12 aa, synthetic construct
  REGION 1..12: note = CDR-H3
DKTYYYYYYMDV

SEQ ID NO: 454 - protein, 19 aa, synthetic construct
  REGION 1..19: note = CDR-H3
EKYFHDKYFHDYYYYYMDV

SEQ ID NO: 455 - protein, 12 aa, synthetic construct
  REGION 1..12: note = CDR-H3
DTSVYYYYYMDV

SEQ ID NO: 456 - protein, 14 aa, synthetic construct
  REGION 1..14: note = CDR-H3
EKILPYYYYYYMDV

SEQ ID NO: 457 - protein, 21 aa, synthetic construct
  REGION 1..21: note = CDR-H3
QWIYIYINPRGFIFLHDAFDI

SEQ ID NO: 458 - protein, 12 aa, synthetic construct
  REGION 1..12: note = CDR-H3
EDVDFHHDAFDI

SEQ ID NO: 459 - protein, 19 aa, synthetic construct
  REGION 1..19: note = CDR-H3
EGVDKNTSTIYYYYYYMDV

SEQ ID NO: 460 - protein, 8 aa, synthetic construct
  REGION 1..8: note = CDR-H3
DRRGYFDL

SEQ ID NO: 461 - protein, 5 aa, synthetic construct
  REGION 1..5: note = CDR-H1
DYYMH

SEQ ID NO: 462 - protein, 17 aa, synthetic construct
  REGION 1..17: note = CDR-H2
LVDPEDGETIYAEKFQG

SEQ ID NO: 463 - protein, 15 aa, synthetic construct
  REGION 1..15: note = CDR-H3
GIHVDIRSMEDWFDP

SEQ ID NO: 464 - protein, 12 aa, synthetic construct
  REGION 1..12: note = CDR-H3
DIGTSYYYYMDV

SEQ ID NO: 465 - protein, 15 aa, synthetic construct
  REGION 1..15: note = CDR-H3
EVVEVFLYYYYYMDV

SEQ ID NO: 466 - protein, 12 aa, synthetic construct
  REGION 1..12: note = CDR-H3
DLYYYYYYYMDV

SEQ ID NO: 467 - protein, 22 aa, synthetic construct
  REGION 1..22: note = CDR-H3
ESWKYFYPRGSIFIHYYYYMDV

SEQ ID NO: 468 - protein, 14 aa, synthetic construct
  REGION 1..14: note = CDR-H3
DRIVEVFYYYYMDV

SEQ ID NO: 469 - protein, 16 aa, synthetic construct
  REGION 1..16: note = CDR-H3
EKYFHDWLYYYYYMDV

SEQ ID NO: 470 - protein, 16 aa, synthetic construct
  REGION 1..16: note = CDR-H3
DLVDKNTSYYYYYMDV

SEQ ID NO: 471 - protein, 8 aa, synthetic construct
  REGION 1..8: note = CDR-H3
VQNEYFQH

SEQ ID NO: 472 - protein, 6 aa, synthetic construct
  REGION 1..6: note = CDR-H3
ANWFDP

SEQ ID NO: 473 - protein, 12 aa, synthetic construct
  REGION 1..12: note = CDR-H3
GGWTAWYYYMDV

SEQ ID NO: 474 - protein, 8 aa, synthetic construct
  REGION 1..8: note = CDR-H3
AKYYYMDV

SEQ ID NO: 475 - protein, 14 aa, synthetic construct
  REGION 1..14: note = CDR-H3
DQVDKNTYYYYMDV

SEQ ID NO: 476 - protein, 10 aa, synthetic construct
  REGION 1..10: note = CDR-H3
ACQLAEYFQH

SEQ ID NO: 477 - protein, 14 aa, synthetic construct
  REGION 1..14: note = CDR-H3
DRVDKNTSYYYMDV

SEQ ID NO: 478 - protein, 12 aa, synthetic construct
  REGION 1..12: note = CDR-H3
RVQLKLVHWFDP

SEQ ID NO: 479 - protein, 6 aa, synthetic construct
  REGION 1..6: note = CDR-H1
SSNWWG

SEQ ID NO: 480 - protein, 5 aa, synthetic construct
  REGION 1..5: note = CDR-H1
SYDIN

SEQ ID NO: 481 - protein, 17 aa, synthetic construct
  REGION 1..17: note = CDR-H2
WMNPNSGNTGYAQKFQG

SEQ ID NO: 482 - protein, 12 aa, synthetic construct
  REGION 1..12: note = CDR-H3
YYDYVTVFYFQH

SEQ ID NO: 483 - protein, 7 aa, synthetic construct
  REGION 1..7: note = CDR-H1
SGGYSWS

SEQ ID NO: 484 - protein, 16 aa, synthetic construct
  REGION 1..16: note = CDR-H2
YIYHSGSTYYNPSLKS

SEQ ID NO: 485 - protein, 11 aa, synthetic construct
  REGION 1..11: note = CDR-H3
ESYPSFYAFDI

SEQ ID NO: 486 - DNA, 81 bp, Homo sapiens; identical to SEQ ID NO: 233
  source 1..81: mol_type = unassigned DNA

SEQ ID NO: 487 - protein, 42 aa, Homo sapiens
KRGRKKLLYIFKQPFMRPVQTTQEEDGCSCRFPEEEEGGCEL

SEQ ID NO: 488 - DNA, 126 bp, Homo sapiens
  source 1..126: mol_type = unassigned DNA
aaacggggcagaaagaaactcctgtatatattcaaacaaccatttatgaggccagtacaaactactcaagaggaagatggctgtagctgccgatttccagaagaagaagaaggaggatgtgaactg

SEQ ID NO: 489 - protein, 195 aa, synthetic construct
  REGION 1..195: note = CD28, 4-1BB and CD3z domains
RSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 490 - DNA, 585 bp, synthetic construct
  misc_feature 1..585: note = CD28, 4-1BB and CD3z domains
  source 1..585: mol_type = other DNA
aggagcaagcggagtcgactgctgcacagcgactacatgaacatgaccccccggaggcctggccccacccggaagcactaccagccctacgcccctcccagggatttcgccgcctaccggagcaaacggggcagaaagaaactcctgtatatattcaaacaaccatttatgaggccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagcgtagaggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggactcagtacagccaccaaggacacctacgacgcccttcacatgcaggccctgccccctcgc

SEQ ID NO: 491 - protein, 17 aa, synthetic construct
  REGION 1..17: note = linker
GGGGSGGGGSGGGGSGG

SEQ ID NO: 492 - protein, 20 aa, synthetic construct
  REGION 1..20: note = self cleaving peptide
GSGEGRGSLLTCGDVEENPG

SEQ ID NO: 493 - DNA, 63 bp, synthetic construct
  misc_feature 1..63: note = self cleaving peptide
  source 1..63: mol_type = other DNA
ggatccggagagggcagaggcagcctgctgacatgtggcgacgtggaagagaaccctggcccc

SEQ ID NO: 494 - RNA, 943 nt, Homo sapiens
  source 1..943: mol_type = unassigned RNA
attcctgaagctgacagcattcgggccgagatgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgtaagcagcatcatggaggtttgaagatgccgcatttggattggatgaattccaaattctgcttgcttgctttttaatattgatatgcttatacacttacactttatgcacaaaatgtagggttataataatgttaacatggacatgatcttctttataattctactttgagtgctgtctccatgtttgatgtatctgagcaggttgctccacaggtagctctaggagggctggcaacttagaggtggggagcagagaattctcttatccaacatcaacatcttggtcagatttgaactcttcaatctcttgcactcaaagcttgttaagatagttaagcgtgcataagttaacttccaatttacatactctgcttagaatttgggggaaaatttagaaatataattgacaggattattggaaatttgttataatgaatgaaacattttgtcatataagattcatatttacttcttatacatttgataaagtaaggcatggttgtggttaatctggtttatttttgttccacaagttaaataaatcataaaacttga

SEQ ID NO: 495 - DNA, 6654 bp, Homo sapiens
  source 1..6654: mol_type = unassigned DNA
aagtggaggcgtcgcgctggcgggcattcctgaagctgacagcattcgggccgagatgtctcgctccgtggccttagctgtgctcgcgctactctctctttctggcctggaggctatccagcgtgagtctctcctaccctcccgctctggtccttcctctcccgctctgcaccctctgtggccctcgctgtgctctctcgctccgtgacttcccttctccaagttctccttggtggcccgccgtggggctagtccagggctggatctcggggaagcggcggggtggcctgggagtggggaagggggtgcgcacccgggacgcgcgctacttgcccctttcggcggggagcaggggagacctttggcctacggcgacgggagggtcgggacaaagtttagggcgtcgataagcgtcagagcgccgaggttgggggagggtttctcttccgctctttcgcggggcctctggctcccccagcgcagctggagtgggggacgggtaggctcgtcccaaaggcgcggcgctgaggtttgtgaacgcgtggaggggcgcttggggtctgggggaggcgtcgcccgggtaagcctgtctgctgcggctctgcttcccttagactggagagctgtggacttcgtctaggcgcccgctaagttcgcatgtcctagcacctctgggtctatgtggggccacaccgtggggaggaaacagcacgcgacgtttgtagaatgcttggctgtgatacaaagcggtttcgaataattaacttatttgttcccatcacatgtcacttttaaaaaattataagaactacccgttattgacatctttctgtgtgccaaggactttatgtgctttgcgtcatttaattttgaaaacagttatcttccgccatagataactactatggttatcttctgcctctcacagatgaagaaactaaggcaccgagattttaagaaacttaattacacaggggataaatggcagcaatcgagattgaagtcaagcctaaccagggcttttgcgggagcgcatgccttttggctgtaattcgtgcatttttttttaagaaaaacgcctgccttctgcgtgagattctccagagcaaactgggcggcatgggccctgtggtcttttcgtacagagggcttcctctttggctctttgcctggttgtttccaagatgtactgtgcctcttactttcggttttgaaaacatgagggggttgggcgtggtagcttacgcctgtaatcccagcacttagggaggccgaggcgggaggatggcttgaggtccgtagttgagaccagcctggccaacatggtgaagcctggtctctacaaaaaataataacaaaaattagccgggtgtggtggctcgtgcctgtggtcccagctgctccggtggctgaggcgggaggatctcttgagcttaggcttttgagctatcatggcgccagtgcactccagcgtgggcaacagagcgagaccctgtctctcaaaaaagaaaaaaaaaaaaaaagaaagagaaaagaaaagaaagaaagaagtgaaggtttgtcagtcaggggagctgtaaaaccattaataaagataatccaagatggttaccaagactgttgaggacgccagagatcttgagcactttctaagtacctggcaatacactaagcgcgctcaccttttcctctggcaaaacatgatcgaaagcagaatgttttgatcatgagaaaattgcatttaatttgaatacaatttatttacaacataaaggataatgtatatatcaccaccattactggtatttgctggttatgttagatgtcattttaaaaaataacaatctgatatttaaaaaaaaatcttattttgaaaatttccaaagtaatacatgccatgcatagaccatttctggaagataccacaagaaacatgtaatgatgattgcctctgaaggtctattttcctcctctgacctgtgtgtgggttttgtttttgttttactgtgggcataaattaatttttcagttaagttttggaagcttaaataactctccaaaagtcataaagccagtaactggttgagcccaaattcaaacccagcctgtctgatacttgtcctcttcttagaaaagattacagtgatgctctcacaaaatcttgccgccttccctcaaacagagagttccaggcaggatgaatctgtgctctgatccctgaggcatttaatatgttcttattattagaagctcagatgcaaagagctctcttagcttttaatgttatgaaaaaaatcaggtcttcattagattccccaatccacctcttgatggggctagtagcctttccttaatgatagggtgtttctagagagatatatctggtcaaggtggcctggtactcctccttctccccacagcctcccagacaaggaggagtagctgccttttagtgatcatgtaccctgaatataagtgtatttaaaagaattttatacacatatatttagtgtcaatctgtatatttagtagcactaacacttctcttcattttcaatgaaaaatatagagtttataatattttcttcccacttccccatggatggtctagtcatgcctctcattttggaaagtactgtttctgaaacattaggcaatatattcccaacctggctagtttacagcaatcacctgtggatgctaattaaaacgcaaatcccactgtcacatgcattactccatttgatcataatggaaagtatgttctgtcccatttgccatagtcctcacctatccctgttgtattttatcgggtccaactcaaccatttaaggtatttgccagctcttgtatgcatttaggttttgtttctttgttttttagctcatgaaattaggtacaaagtcagagaggggtctggcatataaaacctcagcagaaataaagaggttttgttgtttggtaagaacataccttgggttggttgggcacggtggctcgtgcctgtaatcccaacactttgggaggccaaggcaggctgatcacttgaagttgggagttcaagaccagcctggccaacatggtgaaatcccgtctctactgaaaatacaaaaattaaccaggcatggtggtgtgtgcctgtagtcccaggaatcacttgaacccaggaggcggaggttgcagtgagctgagatctcaccactgcacactgcactccagcctgggcaatggaatgagattccatcccaaaaaataaaaaaataaaaaaataaagaacataccttgggttgatccacttaggaacctcagataataacatctgccacgtatagagcaattgctatgtcccaggcactctactagacacttcatacagtttagaaaatcagatgggtgtagatcaaggcaggagcaggaaccaaaaagaaaggcataaacataagaaaaaaaatggaaggggtggaaacagagtacaataacatgagtaatttgatgggggctattatgaactgagaaatgaactttgaaaagtatcttggggccaaatcatgtagactcttgagtgatgtgttaaggaatgctatgagtgctgagagggcatcagaagtccttgagagcctccagagaaaggctcttaaaaatgcagcgcaatctccagtgacagaagatactgctagaaatctgctagaaaaaaaacaaaaaaggcatgtatagaggaattatgagggaaagataccaagtcacggtttattcttcaaaatggaggtggcttgttgggaaggtggaagctcatttggccagagtggaaatggaattgggagaaatcgatgaccaaatgtaaacacttggtgcctgatatagcttgacaccaagttagccccaagtgaaataccctggcaatattaatgtgtcttttcccgatattcctcaggtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtggggtaagtcttacattcttttgtaagctgctgaaagttgtgtatgagtagtcatatcataaagctgctttgatataaaaaaggtctatggccatactaccctgaatgagtcccatcccatctgatataaacaatctgcatattgggattgtcagggaatgttcttaaagatcagattagtggcacctgctgagatactgatgcacagcatggtttctgaaccagtagtttccctgcagttgagcagggagcagcagcagcacttgcacaaatacatatacactcttaacacttcttacctactggcttcctctagcttttgtggcagcttcaggtatatttagcactgaacgaacatctcaagaaggtataggcctttgtttgtaagtcctgctgtcctagcatcctataatcctggacttctccagtactttctggctggattggtatctgaggctagtaggaagggcttgttcctgctgggtagctctaaacaatgtattcatgggtaggaacagcagcctattctgccagccttatttctaaccattttagacatttgttagtacatggtattttaaaagtaaaacttaatgtcttccttttttttctccactgtctttttcatagatcgagacatgtaagcagcatcatggaggtaagtttttgaccttgagaaaatgtttttgtttcactgtcctgaggactatttatagacagctctaacatgataaccctcactatgtggagaacattgacagagtaacattttagcagggaaagaagaatcctacagggtcatgttcccttctcctgtggagtggcatgaagaaggtgtatggccccaggtatggccatattactgaccctctacagagagggcaaaggaactgccagtatggtattgcaggataaaggcaggtggttacccacattacctgcaaggctttgatctttcttctgccatttccacattggacatctctgctgaggagagaaaatgaaccactcttttcctttgtataatgttgttttattcttcagacagaagagaggagttatacagctctgcagacatcccattcctgtatggggactgtgtttgcctcttagaggttcccaggccactagaggagataaagggaaacagattgttataacttgatataatgatactataatagatgtaactacaaggagctccagaagcaagagagagggaggaacttggacttctctgcatctttagttggagtccaaaggcttttcaatgaaattctactgcccagggtacattgatgctgaaaccccattcaaatctcctgttatattctagaacagggaattgatttgggagagcatcaggaaggtggatgatctgcccagtcacactgttagtaaattgtagagccaggacctgaactctaatatagtcatgtgttacttaatgacggggacatgttctgagaaatgcttacacaaacctaggtgttgtagcctactacacgcataggctacatggtatagcctattgctcctagactacaaacctgtacagcctgttactgtactgaatactgtgggcagttgtaacacaatggtaagtatttgtgtatctaaacatagaagttgcagtaaaaatatgctattttaatcttatgagaccactgtcatatatacagtccatcattgaccaaaacatcatatcagcattttttcttctaagattttgggagcaccaaagggatacactaacaggatatactctttataatgggtttggagaactgtctgcagctacttcttttaaaaaggtgatctacacagtagaaattagacaagtttggtaatgagatctgcaatccaaataaaataaattcattgctaacctttttcttttcttttcaggtttgaagatgccgcatttggattggatgaattccaaattctgcttgcttgctttttaatattgatatgcttatacacttacactttatgcacaaaatgtagggttataataatgttaacatggacatgatcttctttataattctactttgagtgctgtctccatgtttgatgtatctgagcaggttgctccacaggtagctctaggagggctggcaacttagaggtggggagcagagaattctcttatccaacatcaacatcttggtcagatttgaactcttcaatctcttgcactcaaagcttgttaagatagttaagcgtgcataagttaacttccaatttacatactctgcttagaatttgggggaaaatttagaaatataattgacaggattattggaaatttgttataatgaatgaaacattttgtcatataagattcatatttacttcttatacatttgataaagtaaggcatggttgtggttaatctggtttatttttgttccacaagttaaataaatcataaaacttga

SEQ ID NO: 496 - DNA, 1698 bp, Homo sapiens
  source 1..1698: mol_type = unassigned DNA
cagaagcagaggggtcagggcgaagtcccagggccccaggcgtggctctcagggtctcaggccccgaaggcggtgtatggattggggagtcccagccttggggattccccaactccgcagtttcttttctccctctcccaacctatgtagggtccttcttcctggatactcacgacgcggacccagttctcactcccattgggtgtcgggtttccagagaagccaatcagtgtcgtcgcggtcgcggttctaaagtccgcacgcacccaccgggactcagattctccccagacgccgaggatggccgtcatggcgccccgaaccctcgtcctgctactctcgggggctctggccctgacccagacctgggcgggctctcactccatgaggtatttcttcacatccgtgtcccggcccggccgcggggagccccgcttcatcgcagtgggctacgtggacgacacgcagttcgtgcggttcgacagcgacgccgcgagccagaggatggagccgcgggcgccgtggatagagcaggagggtccggagtattgggacggggagacacggaaagtgaaggcccactcacagactcaccgagtggacctggggaccctgcgcggctactacaaccagagcgaggccggttctcacaccgtccagaggatgtatggctgcgacgtggggtcggactggcgcttcctccgcgggtaccaccagtacgcctacgacggcaaggattacatcgccctgaaagaggacctgcgctcttggaccgcggcggacatggcagctcagaccaccaagcacaagtgggaggcggcccatgtggcggagcagttgagagcctacctggagggcacgtgcgtggagtggctccgcagatacctggagaacgggaaggagacgctgcagcgcacggacgcccccaaaacgcatatgactcaccacgctgtctctgaccatgaagccaccctgaggtgctgggccctgagcttctaccctgcggagatcacactgacctggcagcgggatggggaggaccagacccaggacacggagctcgtggagaccaggcctgcaggggatggaaccttccagaagtgggcggctgtggtggtgccttctggacaggagcagagatacacctgccatgtgcagcatgagggtttgcccaagcccctcaccctgagatgggagccgtcttcccagcccaccatccccatcgtgggcatcattgctggcctggttctctttggagctgtgatcactggagctgtggtcgctgctgtgatgtggaggaggaagagctcagatagaaaaggagggagctactctcaggctgcaagcagtgacagtgcccagggctctgatgtgtctctcacagcttgtaaagtgtgagacagctgccttgtgtgggactgagaggcaagagttgttcctgcccttccctttgtgacttgaagaaccctgactttgtttctgcaaaggcacctgcatgtgtctgtgttcgtgtaggcataatgtgaggaggtggggagaccaccccacccccatgtccaccatgaccctcttcccacgctgacctgtgctccctccccaatcatctttcctgttccagagaggtggggctgaggtgtctccatctctgtctcaacttcatggtgcactgagctgtaacttcttccttccctattaaaa

SEQ ID NO: 497 - DNA, 3217 bp, Homo sapiens
  source 1..3217: mol_type = unassigned DNA
atggccgtcatggcgccccgaaccctcgtcctgctactctcgggggctctggccctgacccagacctgggcgggtgagtgcggggtcgggagggaaacggcctctgtggggagaagcaacgggcccgcctggcgggggcgcaggacccgggaagccgcgccgggaggagggtcgggcgggtctcagccactcctcgtccccaggctctcactccatgaggtatttcttcacatccgtgtcccggcccggccgcggggagccccgcttcatcgcagtgggctacgtggacgacacgcagttcgtgcggttcgacagcgacgccgcgagccagaggatggagccgcgggcgccgtggatagagcaggagggtccggagtattgggacggggagacacggaaagtgaaggcccactcacagactcaccgagtggacctggggaccctgcgcggctactacaaccagagcgaggccggtgagtgaccccggcccggggcgcaggtcacgacctctcatcccccacggacgggccaggtcgcccacagtctccgggtccgagatccgccccgaagccgcgggaccccgagacccttgccccgggagaggcccaggcgcctttacccggtttcattttcagtttaggccaaaaatccccccaggttggtcggggcggggcggggctcgggggaccgggctgaccgcggggtccgggccaggttctcacaccgtccagaggatgtatggctgcgacgtggggtcggactggcgcttcctccgcgggtaccaccagtacgcctacgacggcaaggattacatcgccctgaaagaggacctgcgctctttgaccgcggcggacatggcagctcagaccaccaagcacaagtgggaggcggcccatgtggcggagcagttgagagcctacctggagggcacgtgcgtggagtggctccgcagatacctggagaacgggaaggagacgctgcagcgcacgggtaccaggggccacggggcgcctccctgatcgcctgtagatctcccgggctggcctcccacaaggaggggagacaattgggaccaacactagaatatcgccctccctctggtcctgagggagaggaatcctcctgggtttccagatcctgtaccagagagtgactctgaggttccgccctgctctctgacacaattaagggataaaatctctgaaggaatgacgggaagacgatccctcgaatactgatgagtggttccctttgacacacacaggcagcagccttgggcccgtgacttttcctctcaggccttgttctctgcttcacactcaatgtgtgtgggggtctgagtccagcacttctgagtccttcagcctccactcaggtcaggaccagaagtcgctgttccctcttcagggactagaattttccacggaataggagattatcccaggtgcctgtgtccaggctggtgtctgggttctgtgctcccttccccatcccaggtgtcctgtccattctcaagatagccacatgtgtgctggaggagtgtcccatgacagatgcaaaatgcctgaatgatctgactcttcctgacagacgcccccaaaacgcatatgactcaccacgctgtctctgaccatgaagccaccctgaggtgctgggccctgagcttctaccctgcggagatcacactgacctggcagcgggatggggaggaccagacccaggacacggagctcgtggagaccaggcctgcaggggatggaaccttccagaagtgggcggctgtggtggtgccttctggacaggagcagagatacacctgccatgtgcagcatgagggtttgcccaagcccctcaccctgagatggggtaaggagggagacgggggtgtcatgtcttttagggaaagcaggagcctctctgacctttagcagggtcagggcccctcaccttcccctcttttcccagagccgtcttcccagcccaccatccccatcgtgggcatcattgctggcctggttctctttggagctgtgatcactggagctgtggtcgctgctgtgatgtggaggaggaagagctcaggtggggaaggggtgaagggtgggtctgagatttcttgtctcactgagggttccaagacccaggtagaagtgtgccctgcctcgttactgggaagcaccacccacaattatgggcctacccagcctgggccctgtgtgccagcacttactcttttgtaaagcacctgttaaaatgaaggacagatttatcaccttgattacagcggtgatgggacctgatcccagcagtcacaagtcacaggggaaggtccctgaggaccttcaggagggcggttggtccaggacccacacctgctttcttcatgtttcctgatcccgccctgggtctgcagtcacacatttctggaaacttctctgaggtccaagacttggaggttcctctaggaccttaaggccctgactcctttctggtatctcacaggacattttcttcccacagatagaaaaggagggagctactctcaggctgcaagtaagtatgaaggaggctgatgcctgaggtccttgggatattgtgtttgggagcccatgggggagctcacccaccccacaattcctcctctagccacatcttctgtgggatctgaccaggttctgtttttgttctaccccaggcagtgacagtgcccagggctctgatgtgtctctcacagcttgtaaaggtgagagcctggagggcctgatgtgtgttgggtgttgggcggaacagtggacacagctgtgctatggggtttctttccattggatgtattgagcatgcgatgggctgtttaaagtgtgacccctcactgtgacagatacgaatttgttcatgaatatttttttctatagtgtgagacagctgccttgtgtgggactgagaggcaagagttgttcctgcccttccctttgtgacttgaagaaccctgactttgtttctgcaaaggcacctgcatgtgtctgtgttcgtgtaggcataatgtgaggaggtggggagaccaccccacccccatgtccaccatgaccctcttcccacgctgacctgtgctccctccccaatcatctttcctgttccagagaggtggggctgaggtgtctccatctctgtctcaacttcatggtgcactgagctgtaacttcttccttccctattaaaa

SEQ ID NO: 498 - DNA, 83 bp, synthetic construct
  misc_feature 1..83: note = Cas9 scaffold
  source 1..83: mol_type = other DNA
gttttagagctagaaatagcaagttaaaataaggctagtccgttatcaacttgaaaaagtggcaccgagtcggtgcttttttt

SEQ ID NO: 499 - DNA, 20 bp, synthetic construct
  misc_feature 1..20: note = HLA-A targeting sequence
  source 1..20: mol_type = other DNA
tggacgacacgcagttcgtg

SEQ ID NO: 500 - DNA, 20 bp, synthetic construct
  misc_feature 1..20: note = HLA-A targeting sequence
  source 1..20: mol_type = other DNA
cagatacctggagaacggga

SEQ ID NO: 501 - DNA, 20 bp, synthetic construct
  misc_feature 1..20: note = HLA-A targeting sequence
  source 1..20: mol_type = other DNA
tcccgttctccaggtatctg

SEQ ID NO: 502 - DNA, 20 bp, synthetic construct
  misc_feature 1..20: note = HLA-A targeting sequence
  source 1..20: mol_type = other DNA
ccgccgcggtccaagagcgc

SEQ ID NO: 503 - DNA, 20 bp, synthetic construct
  misc_feature 1..20: note = HLA-A targeting sequence
  source 1..20: mol_type = other DNA
cctgcgctcttggaccgcgg

SEQ ID NO: 504 - DNA, 21 bp, synthetic construct
  misc_feature 1..21: note = HLA-A targeting sequence
  source 1..21: mol_type = other DNA
ggacctgcgctcttggaccgc

SEQ ID NO: 505 - DNA, 23 bp, synthetic construct
  misc_feature 1..23: note = HLA-A targeting sequence
  source 1..23: mol_type = other DNA
aaggagacgctgcagcgcacggg

SEQ ID NO: 506 - DNA, 23 bp, synthetic construct
  misc_feature 1..23: note = HLA-A targeting sequence
  source 1..23: mol_type = other DNA
gaaggagacgctgcagcgcacgg

SEQ ID NO: 507 - DNA, 23 bp, synthetic construct
  misc_feature 1..23: note = HLA-A targeting sequence
  source 1..23: mol_type = other DNA
gcgggcgccgtggatagagcagg

SEQ ID NO: 508 - DNA, 23 bp, synthetic construct
  misc_feature 1..23: note = HLA-A targeting sequence
  source 1..23: mol_type = other DNA
tgctctatccacggcgcccgcgg

SEQ ID NO: 509 - DNA, 23 bp, synthetic construct
  misc_feature 1..23: note = HLA-A targeting sequence
  source 1..23: mol_type = other DNA
cgatgaagcggggctccccgcgg

SEQ ID NO: 510 - DNA, 23 bp, synthetic construct
  misc_feature 1..23: note = HLA-A targeting sequence
  source 1..23: mol_type = other DNA
cgtgtcccggcccggccgcgggg

SEQ ID NO: 511 - DNA, 23 bp, synthetic construct
  misc_feature 1..23: note = HLA-A targeting sequence
  source 1..23: mol_type = other DNA
cggctccatcctctggctcgcgg

SEQ ID NO: 512 - DNA, 23 bp, synthetic construct
  misc_feature 1..23: note = HLA-A targeting sequence
  source 1..23: mol_type = other DNA
gatgtaatccttgccgtcgtagg

SEQ ID NO: 513 - DNA, 23 bp, synthetic construct
  misc_feature 1..23: note = HLA-A targeting sequence
  source 1..23: mol_type = other DNA
acagcgacgccgcgagccagagg

SEQ ID NO: 514 - DNA, 23 bp, synthetic construct
  misc_feature 1..23: note = HLA-A targeting sequence
  source 1..23: mol_type = other DNA
ggatggagccgcgggcgccgtgg

SEQ ID NO: 515 - DNA, 23 bp, synthetic construct
  misc_feature 1..23: note = HLA-A targeting sequence
  source 1..23: mol_type = other DNA
ggcgccgtggatagagcaggagg

SEQ ID NO: 516 - DNA, 23 bp, synthetic construct
  misc_feature 1..23: note = HLA-A targeting sequence
  source 1..23: mol_type = other DNA
gcgccgtggatagagcaggaggg

SEQ ID NO: 517 - DNA, 23 bp, synthetic construct
  misc_feature 1..23: note = HLA-A targeting sequence
  source 1..23: mol_type = other DNA
cggctactacaaccagagcgagg

SEQ ID NO: 518 - DNA, 23 bp, synthetic construct
  misc_feature 1..23: note = HLA-A targeting sequence
  source 1..23: mol_type = other DNA
ctggttgtagtagccgcgcaggg

SEQ ID NO: 519 - DNA, 23 bp, synthetic construct
  misc_feature 1..23: note = HLA-A targeting sequence
  source 1..23: mol_type = other DNA
tactacaaccagagcgaggccgg

SEQ ID NO: 520 - DNA, 23 bp, synthetic construct
  misc_feature 1..23: note = HLA-A targeting sequence
  source 1..23: mol_type = other DNA
ctacctggagggcacgtgcgtgg

SEQ ID NO: 521 - DNA, 23 bp, synthetic construct
  misc_feature 1..23: note = HLA-A targeting sequence
  source 1..23: mol_type = other DNA
cacgcacgtgccctccaggtagg

SEQ ID NO: 522 - DNA, 23 bp, synthetic construct
  misc_feature 1..23: note = HLA-A targeting sequence
  source 1..23: mol_type = other DNA
gcagggtccccaggtccactcgg

SEQ ID NO: 523 - DNA, 23 bp, synthetic construct
  misc_feature 1..23: note = HLA-A targeting sequence
  source 1..23: mol_type = other DNA
gtggacctggggaccctgcgcgg

SEQ ID NO: 524 - DNA, 23 bp, synthetic construct
  misc_feature 1..23: note = HLA-A targeting sequence
  source 1..23: mol_type = other DNA
tggagggcacgtgcgtggagtgg

SEQ ID NO: 525 - DNA, 23 bp, synthetic construct
  misc_feature 1..23: note = HLA-A targeting sequence
  source 1..23: mol_type = other DNA
gtatggctgcgacgtggggtcgg

SEQ ID NO: 526 - DNA, 23 bp, synthetic construct
  misc_feature 1..23: note = HLA-A targeting sequence
  source 1..23: mol_type = other DNA
ctgagctgccatgtccgccgcgg

SEQ ID NO: 527 - DNA, 23 bp, synthetic construct
  misc_feature 1..23: note = HLA-A targeting sequence
  source 1..23: mol_type = other DNA
ggattacatcgccctgaaagagg

SEQ ID NO: 528 - DNA, 23 bp, synthetic construct
  misc_feature 1..23: note = HLA-A targeting sequence
  source 1..23: mol_type = other DNA
caagtgggaggcggcccatgtgg

SEQ ID NO: 529 - DNA, 23 bp, synthetic construct
  misc_feature 1..23: note = HLA-A targeting sequence
  source 1..23: mol_type = other DNA
gtgggaggcggcccatgtggcgg

SEQ ID NO: 530 - DNA, 23 bp, synthetic construct
  misc_feature 1..23: note = HLA-A targeting sequence
  source 1..23: mol_type = other DNA
cagttgagagcctacctggaggg

SEQ ID NO: 531 - DNA, 23 bp, synthetic construct
  misc_feature 1..23: note = HLA-A targeting sequence
  source 1..23: mol_type = other DNA
gcagttgagagcctacctggagg

SEQ ID NO: 532 - DNA, 23 bp, synthetic construct
  misc_feature 1..23: note = HLA-A targeting sequence
  source 1..23: mol_type = other DNA
taccaccagtacgcctacgacgg

SEQ ID NO: 533 - DNA, 23 bp, synthetic construct
  misc_feature 1..23: note = HLA-A targeting sequence
  source 1..23: mol_type = other DNA
tgccgtcgtaggcgtactggtgg

SEQ ID NO: 534 - DNA, 23 bp, synthetic construct
  misc_feature 1..23: note = HLA-A targeting sequence
  source 1..23: mol_type = other DNA
ccagtacgcctacgacggcaagg

SEQ ID NO: 535 - DNA, 23 bp, synthetic construct
  misc_feature 1..23: note = HLA-A targeting sequence
  source 1..23: mol_type = other DNA
ggatgtgaagaaatacctcatgg

SEQ ID NO: 536 - DNA, 23 bp, synthetic construct
  misc_feature 1..23: note = HLA-A targeting sequence
  source 1..23: mol_type = other DNA
atttcttcacatccgtgtcccgg

SEQ ID NO: 537 - DNA, 23 bp, synthetic construct
  misc_feature 1..23: note = HLA-A targeting sequence
  source 1..23: mol_type = other DNA
aggcgtactggtggtacccgcgg

SEQ ID NO: 538 - DNA, 23 bp, synthetic construct
  misc_feature 1..23: note = HLA-A targeting sequence
  source 1..23: mol_type = other DNA
cgtactggtggtacccgcggagg

SEQ ID NO: 539 - DNA, 23 bp, synthetic construct
  misc_feature 1..23: note = HLA-A targeting sequence
  source 1..23: mol_type = other DNA
gaggatgtatggctgcgacgtgg

SEQ ID NO: 540 - DNA, 23 bp, synthetic construct
  misc_feature 1..23: note = HLA-A targeting sequence
  source 1..23: mol_type = other DNA
ggatgtatggctgcgacgtgggg

SEQ ID NO: 541 - DNA, 23 bp, synthetic construct
  misc_feature 1..23: note = HLA-A targeting sequence
  source 1..23: mol_type = other DNA
ctcagaccaccaagcacaagtgg

SEQ ID NO: 542 - DNA, 23 bp, synthetic construct
  misc_feature 1..23: note = HLA-A targeting sequence
  source 1..23: mol_type = other DNA
tcagaccaccaagcacaagtggg

SEQ ID NO: 543 - DNA, 23 bp, synthetic construct
  misc_feature 1..23: note = HLA-A targeting sequence
  source 1..23: mol_type = other DNA
caccaagcacaagtgggaggcgg

SEQ ID NO: 544 - DNA, 23 bp, synthetic construct
  misc_feature 1..23: note = HLA-A targeting sequence
  source 1..23: mol_type = other DNA
gaccaccaagcacaagtgggagg

SEQ ID NO: 545 - DNA, 23 bp, synthetic construct
  misc_feature 1..23: note = HLA-A targeting sequence
  source 1..23: mol_type = other DNA
gagccccgcttcatcgcagtggg

SEQ ID NO: 546 - DNA, 23 bp, synthetic construct
  misc_feature 1..23: note = HLA-A targeting sequence
  source 1..23: mol_type = other DNA
gtagcccactgcgatgaagcggg

SEQ ID NO: 547 - DNA, 23 bp, synthetic construct
  misc_feature 1..23: note = HLA-A targeting sequence
  source 1..23: mol_type = other DNA
tagcccactgcgatgaagcgggg

SEQ ID NO: 548 - DNA, 23 bp, synthetic construct
  misc_feature 1..23: note = HLA-A targeting sequence
  source 1..23: mol_type = other DNA
cgtagcccactgcgatgaagcgg

SEQ ID NO: 549 - DNA, 23 bp, synthetic construct
  misc_feature 1..23: note = HLA-A targeting sequence
  source 1..23: mol_type = other DNA
cttcatcgcagtgggctacgtgg

SEQ ID NO: 550 - DNA, 23 bp, synthetic construct
  misc_feature 1..23: note = HLA-A targeting sequence
  source 1..23: mol_type = other DNA
ggagccccgcttcatcgcagtgg

SEQ ID NO: 551 - DNA, 23 bp, synthetic construct
  misc_feature 1..23: note = HLA-A targeting sequence
  source 1..23: mol_type = other DNA
cggggagacacggaaagtgaagg

SEQ ID NO: 552 - DNA, 23 bp, synthetic construct
  misc_feature 1..23: note = HLA-A targeting sequence
  source 1..23: mol_type = other DNA
agtattgggacggggagacacgg

SEQ ID NO: 553 - DNA, 23 bp, synthetic construct
  misc_feature 1..23: note = HLA-A targeting sequence
  source 1..23: mol_type = other DNA
agggtccggagtattgggacggg

SEQ ID NO: 554 - DNA, 23 bp, synthetic construct
  misc_feature 1..23: note = HLA-A targeting sequence
  source 1..23: mol_type = other DNA
gagggtccggagtattgggacgg

SEQ ID NO: 555 - DNA, 23 bp, synthetic construct
  misc_feature 1..23: note = HLA-A targeting sequence
  source 1..23: mol_type = other DNA
ggaccctcctgctctatccacgg

SEQ ID NO: 556 - DNA, 23 bp, synthetic construct
  misc_feature 1..23: note = HLA-A targeting sequence
  source 1..23: mol_type = other DNA
gtggatagagcaggagggtccgg

SEQ ID NO: 557 - DNA, 23 bp, synthetic construct
  misc_feature 1..23: note = HLA-A targeting sequence
  source 1..23: mol_type = other DNA
agactcaccgagtggacctgggg

SEQ ID NO: 558 - DNA, 23 bp, synthetic construct
  misc_feature 1..23: note = HLA-A targeting sequence
  source 1..23: mol_type = other DNA
cactcggtgagtctgtgagtggg

SEQ ID NO: 559 - DNA, 23 bp, synthetic construct
  misc_feature 1..23: note = HLA-A targeting sequence
  source 1..23: mol_type = other DNA
cagactcaccgagtggacctggg

SEQ ID NO: 560 - DNA, 23 bp, synthetic construct
  misc_feature 1..23: note = HLA-A targeting sequence
  source 1..23: mol_type = other DNA
ccactcacagactcaccgagtgg

SEQ ID NO: 561 - DNA, 23 bp, synthetic construct
  misc_feature 1..23: note = HLA-A targeting sequence
  source 1..23: mol_type = other DNA
ccactcggtgagtctgtgagtgg

SEQ ID NO: 562 - DNA, 23 bp, synthetic construct
  misc_feature 1..23: note = HLA-A targeting sequence
  source 1..23: mol_type = other DNA
tcggactggcgcttcctccgcgg

SEQ ID NO: 563 - DNA, 23 bp, synthetic construct
  misc_feature 1..23: note = HLA-A targeting sequence
  source 1..23: mol_type = other DNA
gcagccatacatcctctggacgg

SEQ ID NO: 564 - DNA, 23 bp, synthetic construct
  misc_feature 1..23: note = HLA-A targeting sequence
  source 1..23: mol_type = other DNA
tctcaactgctccgccacatggg

SEQ ID NO: 565 - DNA, 23 bp, synthetic construct
  misc_feature 1..23: note = HLA-A targeting sequence
  source 1..23: mol_type = other DNA
accctcatgctgcacatggcagg

SEQ ID NO: 566 - DNA, 23 bp, synthetic construct
  misc_feature 1..23: note = HLA-A targeting sequence
  source 1..23: mol_type = other DNA
acctgccatgtgcagcatgaggg

SEQ ID NO: 567 - DNA, 23 bp, synthetic construct
  misc_feature 1..23: note = HLA-A targeting sequence
  source 1..23: mol_type = other DNA
cacctgccatgtgcagcatgagg

SEQ ID NO: 568 - DNA, 23 bp, synthetic construct
  misc_feature 1..23: note = HLA-A targeting sequence
  source 1..23: mol_type = other DNA
ggaggaccagacccaggacacgg

SEQ ID NO: 569 - DNA, 23 bp, synthetic construct
  misc_feature 1..23: note = HLA-A targeting sequence
  source 1..23: mol_type = other DNA
ggatggggaggaccagacccagg

SEQ ID NO: 570 - DNA, 23 bp, synthetic construct
  misc_feature 1..23: note = HLA-A targeting sequence
  source 1..23: mol_type = other DNA
gacctggcagcgggatggggagg

SEQ ID NO: 571 - DNA, 23 bp, synthetic construct
  misc_feature 1..23: note = HLA-A targeting sequence
  source 1..23: mol_type = other DNA
agatcacactgacctggcagcgg

SEQ ID NO: 572 - DNA, 23 bp, synthetic construct
  misc_feature 1..23: note = HLA-A targeting sequence
  source 1..23: mol_type = other DNA
gatcacactgacctggcagcggg

SEQ ID NO: 573 - DNA, 23 bp, synthetic construct
  misc_feature 1..23: note = HLA-A targeting sequence
  source 1..23: mol_type = other DNA
aggtcagtgtgatctccgcaggg

SEQ ID NO: 574 - DNA, 23 bp, synthetic construct
  misc_feature 1..23: note = HLA-A targeting sequence
  source 1..23: mol_type = other DNA
aagcccctcaccctgagatgggg

SEQ ID NO: 575 - DNA, 23 bp, synthetic construct
  misc_feature 1..23: note = HLA-A targeting sequence
  source 1..23: mol_type = other DNA
ctgcggagatcacactgacctgg

SEQ ID NO: 576 - DNA, 23 bp, synthetic construct
  misc_feature 1..23: note = HLA-A targeting sequence
  source 1..23: mol_type = other DNA
cagcaatgatgcccacgatgggg

SEQ ID NO: 577 - DNA, 23 bp, synthetic construct
  misc_feature 1..23: note = HLA-A targeting sequence
  source 1..23: mol_type = other DNA
ccagcaatgatgcccacgatggg

SEQ ID NO: 578 - DNA, 23 bp, synthetic construct
  misc_feature 1..23: note = HLA-A targeting sequence
  source 1..23: mol_type = other DNA
gccagcaatgatgcccacgatgg

SEQ ID NO: 579 - DNA, 23 bp, synthetic construct
  misc_feature 1..23: note = HLA-A targeting sequence
  source 1..23: mol_type = other DNA
ggatggaaccttccagaagtggg

SEQ ID NO: 580 - DNA, 23 bp, synthetic construct
  misc_feature 1..23: note = HLA-A targeting sequence
  source 1..23: mol_type = other DNA
gggatggaaccttccagaagtgg

SEQ ID NO: 581 - DNA, 23 bp, synthetic construct
  misc_feature 1..23: note = HLA-A targeting sequence
  source 1..23: mol_type = other DNA
atgcccacgatggggatggtggg

SEQ ID NO: 582 - DNA, 23 bp, synthetic construct
  misc_feature 1..23: note = HLA-A targeting sequence
  source 1..23: mol_type = other DNA
cagcccaccatccccatcgtggg

SEQ ID NO: 583 - DNA, 23 bp, synthetic construct
  misc_feature 1..23: note = HLA-A targeting sequence
  source 1..23: mol_type = other DNA
ccagcccaccatccccatcgtgg

SEQ ID NO: 584 - DNA, 23 bp, synthetic construct
  misc_feature 1..23: note = HLA-A targeting sequence
  source 1..23: mol_type = other DNA
gatgcccacgatggggatggtgg

SEQ ID NO: 585 - DNA, 23 bp, synthetic construct
  misc_feature 1..23: note = HLA-A targeting sequence
  source 1..23: mol_type = other DNA
cagggcccagcacctcagggtgg

SEQ ID NO: 586 - DNA, 23 bp, synthetic construct
  misc_feature 1..23: note = HLA-A targeting sequence
  source 1..23: mol_type = other DNA
aatgatgcccacgatggggatgg

SEQ ID NO: 587 - DNA, 23 bp, synthetic construct
  misc_feature 1..23: note = HLA-A targeting sequence
  source 1..23: mol_type = other DNA
ggccctgacccagacctgggcgg

SEQ ID NO: 588 - DNA, 23 bp, synthetic construct
  misc_feature 1..23: note = HLA-A targeting sequence
  source 1..23: mol_type = other DNA
gacccaggacacggagctcgtgg

SEQ ID NO: 589 - DNA, 23 bp, synthetic construct
  misc_feature 1..23: note = HLA-A targeting sequence
  source 1..23: mol_type = other DNA
acacggagctcgtggagaccagg

SEQ ID NO: 590 - DNA, 23 bp, synthetic construct
  misc_feature 1..23: note = HLA-A targeting sequence
  source 1..23: mol_type = other DNA
cgtggagaccaggcctgcagggg

SEQ ID NO: 591 - DNA, 23 bp, synthetic construct
  misc_feature 1..23: note = HLA-A targeting sequence
  source 1..23: mol_type = other DNA
tcgtggagaccaggcctgcaggg

SEQ ID NO: 592 - DNA, 23 bp, synthetic construct
  misc_feature 1..23: note = HLA-A targeting sequence
  source 1..23: mol_type = other DNA
agctgtgatcactggagctgtgg

SEQ ID NO: 593 - DNA, 23 bp, synthetic construct
  misc_feature 1..23: note = HLA-A targeting sequence
  source 1..23: mol_type = other DNA
aaaaggagggagctactctcagg

SEQ ID NO: 594 - DNA, 23 bp, synthetic construct
  misc_feature 1..23: note = HLA-A targeting sequence
  source 1..23: mol_type = other DNA
atgtggaggaggaagagctcagg

SEQ ID NO: 595 - DNA, 23 bp, synthetic construct
  misc_feature 1..23: note = HLA-A targeting sequence
  source 1..23: mol_type = other DNA
gtgtctctcacagcttgtaaagg

SEQ ID NO: 596 - DNA, 23 bp, synthetic construct
  misc_feature 1..23: note = HLA-A targeting sequence
  source 1..23: mol_type = other DNA
gagagacacatcagagccctggg

SEQ ID NO: 597 - DNA, 23 bp, synthetic construct
  misc_feature 1..23: note = HLA-A targeting sequence
  source 1..23: mol_type = other DNA
ctccgcagggtagaagctcaggg

SEQ ID NO: 598 - DNA, 23 bp, synthetic construct
  misc_feature 1..23: note = HLA-A targeting sequence
  source 1..23: mol_type = other DNA
ggccctgagcttctaccctgcgg

SEQ ID NO: 599 - DNA, 23 bp, synthetic construct
  misc_feature 1..23: note = HLA-A targeting sequence
  source 1..23: mol_type = other DNA
gctcagggcccagcacctcaggg

SEQ ID NO: 600 - DNA, 23 bp, synthetic construct
  misc_feature 1..23: note = HLA-A targeting sequence
  source 1..23: mol_type = other DNA
tatctctgctcctgtccagaagg

SEQ ID NO: 601 - DNA, 23 bp, synthetic construct
  misc_feature 1..23: note = HLA-A targeting sequence
  source 1..23: mol_type = other DNA
agtagcaggacgagggttcgggg

SEQ ID NO: 602 - DNA, 23 bp, synthetic construct
  misc_feature 1..23: note = HLA-A targeting sequence
  source 1..23: mol_type = other DNA
ccccgagagtagcaggacgaggg

SEQ ID NO: 603 - DNA, 23 bp, synthetic construct
  misc_feature 1..23: note = HLA-A targeting sequence
  source 1..23: mol_type = other DNA
ccctcgtcctgctactctcgggg

SEQ ID NO: 604 - DNA, 23 bp, synthetic construct
  misc_feature 1..23: note = HLA-A targeting sequence
  source 1..23: mol_type = other DNA
cctcgtcctgctactctcggggg

SEQ ID NO: 605 - DNA, 23 bp, synthetic construct
  misc_feature 1..23: note = HLA-A targeting sequence
  source 1..23: mol_type = other DNA
ctgtggtcgctgctgtgatgtgg

SEQ ID NO: 606 - DNA, 23 bp, synthetic construct
  misc_feature 1..23: note = HLA-A targeting sequence
  source 1..23: mol_type = other DNA
tcgctgctgtgatgtggaggagg

SEQ ID NO: 607 - DNA, 23 bp, synthetic construct
  misc_feature 1..23: note = HLA-A targeting sequence
  source 1..23: mol_type = other DNA
tggtcgctgctgtgatgtggagg

SEQ ID NO: 608 - DNA, 23 bp, synthetic construct
  misc_feature 1..23: note = HLA-A targeting sequence
  source 1..23: mol_type = other DNA
cacagccgcccacttctggaagg

SEQ ID NO: 609 - DNA, 23 bp, synthetic construct
  misc_feature 1..23: note = HLA-A targeting sequence
  source 1..23: mol_type = other DNA
ccagaagtgggcggctgtggtgg

SEQ ID NO: 610 - DNA, 23 bp, synthetic construct
  misc_feature 1..23: note = HLA-A targeting sequence
  source 1..23: mol_type = other DNA
tggaaccttccagaagtgggcgg

SEQ ID NO: 611 - DNA, 23 bp, synthetic construct
  misc_feature 1..23: note = HLA-A targeting sequence
  source 1..23: mol_type = other DNA
tcacagctccaaagagaaccagg

SEQ ID NO: 612 - DNA, 23 bp, synthetic construct
  misc_feature 1..23: note = HLA-A targeting sequence
  source 1..23: mol_type = other DNA
ctgaccatgaagccaccctgagg

SEQ ID NO: 613 - DNA, 23 bp, synthetic construct
  misc_feature 1..23: note = HLA-A targeting sequence
  source 1..23: mol_type = other DNA
gcaaaccctcatgctgcacatgg

SEQ ID NO: 614 - DNA, 23 bp, synthetic construct
  misc_feature 1..23: note = HLA-A targeting sequence
  source 1..23: mol_type = other DNA
tgaagccaccctgaggtgctggg

SEQ ID NO: 615 - DNA, 23 bp, synthetic construct
  misc_feature 1..23: note = HLA-A targeting sequence
  source 1..23: mol_type = other DNA
ggtgagtcatatgcgttttgggg

SEQ ID NO: 616 - DNA, 23 bp, synthetic construct
  misc_feature 1..23: note = HLA-A targeting sequence
  source 1..23: mol_type = other DNA
gtgagtcatatgcgttttggggg

SEQ ID NO: 617 - DNA, 23 bp, synthetic construct
  misc_feature 1..23: note = HLA-A targeting sequence
  source 1..23: mol_type = other DNA
cttcatggtcagagacagcgtgg

SEQ ID NO: 618 - DNA, 23 bp, synthetic construct
  misc_feature 1..23: note = HLA-A targeting sequence
  source 1..23: mol_type = other DNA
tctggccctgacccagacctggg

SEQ ID NO: 619 - DNA, 20 bp, synthetic construct
  misc_feature 1..20: note = B2M targeting sequences
  source 1..20: mol_type = other DNA
cgcgagcacagctaaggcca

SEQ ID NO: 620 - DNA, 20 bp, synthetic construct
  misc_feature 1..20: note = B2M targeting sequences
  source 1..20: mol_type = other DNA
gagtagcgcgagcacagcta

SEQ ID NO: 621 - DNA, 20 bp, synthetic construct
  misc_feature 1..20: note = B2M targeting sequences
  source 1..20: mol_type = other DNA
agggtaggagagactcacgc

SEQ ID NO: 622 - DNA, 20 bp, synthetic construct
  misc_feature 1..20: note = B2M targeting sequences
  source 1..20: mol_type = other DNA
ctgaatctttggagtacctg

SEQ ID NO: 623 - DNA, 20 bp, synthetic construct
  misc_feature 1..20: note = B2M targeting sequences
  source 1..20: mol_type = other DNA
tcacgtcatccagcagagaa

SEQ ID NO: 624 - DNA, 20 bp, synthetic construct
  misc_feature 1..20: note = B2M targeting sequences
  source 1..20: mol_type = other DNA
tcctgaattgctatgtgtct

SEQ ID NO: 625 - DNA, 20 bp, synthetic construct
  misc_feature 1..20: note = B2M targeting sequences
  source 1..20: mol_type = other DNA
aagtcaacttcaatgtcgga

SEQ ID NO: 626 - DNA, 20 bp, synthetic construct
  misc_feature 1..20: note = B2M targeting sequences
  source 1..20: mol_type = other DNA
gtcttttcccgatattcctc

SEQ ID NO: 627 - DNA, 20 bp, synthetic construct
  misc_feature 1..20: note = B2M targeting sequences
  source 1..20: mol_type = other DNA
tggagtacctgaggaatatc

SEQ ID NO: 628 - DNA, 20 bp, synthetic construct
  misc_feature 1..20: note = B2M targeting sequences
  source 1..20: mol_type = other DNA
cagcccaagatagttaagtg

SEQ ID NO: 629 - DNA, 20 bp, synthetic construct
  misc_feature 1..20: note = B2M targeting sequences
  source 1..20: mol_type = other DNA
acaaagtcacatggttcaca

SEQ ID NO: 630 - DNA, 20 bp, synthetic construct
  misc_feature 1..20: note = B2M targeting sequences
  source 1..20: mol_type = other DNA
actctctctttctggcctgg

SEQ ID NO: 631 - DNA, 20 bp, synthetic construct
  misc_feature 1..20: note = B2M targeting sequences
  source 1..20: mol_type = other DNA
tgggctgtgacaaagtcaca

SEQ ID NO: 632 - DNA, 20 bp, synthetic construct
  misc_feature 1..20: note = B2M targeting sequences
  source 1..20: mol_type = other DNA
ggccgagatgtctcgctccg

SEQ ID NO: 633 - DNA, 20 bp, synthetic construct
  misc_feature 1..20: note = B2M targeting sequences
  source 1..20: mol_type = other DNA
cagtaagtcaacttcaatgt

SEQ ID NO: 634 - DNA, 20 bp, synthetic construct
  misc_feature 1..20: note = B2M targeting sequences
  source 1..20: mol_type = other DNA
actcacgctggatagcctcc

SEQ ID NO: 635 - DNA, 20 bp, synthetic construct
  misc_feature 1..20: note = B2M targeting sequences
  source 1..20: mol_type = other DNA
catactcatctttttcagtg

SEQ ID NO: 636 - DNA, 20 bp, synthetic construct
  misc_feature 1..20: note = B2M targeting sequences
  source 1..20: mol_type = other DNA
cacagcccaagatagttaag

SEQ ID NO: 637 - DNA, 20 bp, synthetic construct
  misc_feature 1..20: note = B2M targeting sequences
  source 1..20: mol_type = other DNA
ttcagacttgtctttcagca

SEQ ID NO: 638 - DNA, 20 bp, synthetic construct
  misc_feature 1..20: note = B2M targeting sequences
  source 1..20: mol_type = other DNA
agtcacatggttcacacggc

SEQ ID NO: 639 - DNA, 20 bp, synthetic construct
  misc_feature 1..20: note = B2M targeting sequences
  source 1..20: mol_type = other DNA
atactcatctttttcagtgg

SEQ ID NO: 640 - DNA, 20 bp, synthetic construct
  misc_feature 1..20: note = B2M targeting sequences
  source 1..20: mol_type = other DNA
ggcatactcatctttttcag

SEQ ID NO: 641 - DNA, 20 bp, synthetic construct
  misc_feature 1..20: note = B2M targeting sequences
  source 1..20: mol_type = other DNA
acagcccaagatagttaagt

SEQ ID NO: 642 - DNA, 20 bp, synthetic construct
  misc_feature 1..20: note = B2M targeting sequences
  source 1..20: mol_type = other DNA
gctactctctctttctggcc

SEQ ID NO: 643 - DNA, 20 bp, synthetic construct
  misc_feature 1..20: note = B2M targeting sequences
  source 1..20: mol_type = other DNA
tggagagagaattgaaaaag

SEQ ID NO: 644 - DNA, 20 bp, synthetic construct
  misc_feature 1..20: note = B2M targeting sequences
  source 1..20: mol_type = other DNA
acttgtctttcagcaaggac

SEQ ID NO: 645 - DNA, 20 bp, synthetic construct
  misc_feature 1..20: note = B2M targeting sequences
  source 1..20: mol_type = other DNA
gaagttgacttactgaagaa

SEQ ID NO: 646 - DNA, 20 bp, synthetic construct
  misc_feature 1..20: note = B2M targeting sequences
  source 1..20: mol_type = other DNA
ggccacggagcgagacatct

SEQ ID NO: 647 - DNA, 20 bp, synthetic construct
  misc_feature 1..20: note = B2M targeting sequences
  source 1..20: mol_type = other DNA
gcatactcatctttttcagt

SEQ ID NO: 648 - DNA, 20 bp, synthetic construct
  misc_feature 1..20: note = B2M targeting sequences
  source 1..20: mol_type = other DNA
cgtgagtaaacctgaatctt

SEQ ID NO: 649 - DNA, 20 bp, synthetic construct
  misc_feature 1..20: note = B2M targeting sequences
  source 1..20: mol_type = other DNA
ttaccccacttaactatctt

SEQ ID NO: 650 - DNA, 20 bp, synthetic construct
  misc_feature 1..20: note = B2M targeting sequences
  source 1..20: mol_type = other DNA
ttggagtacctgaggaatat

SEQ ID NO: 651 - DNA, 20 bp, synthetic construct
  misc_feature 1..20: note = B2M targeting sequences
  source 1..20: mol_type = other DNA
acccagacacatagcaattc

SEQ ID NO: 652 - DNA, 20 bp, synthetic construct
  misc_feature 1..20: note = B2M targeting sequences
  source 1..20: mol_type = other DNA
tttgactttccattctctgc

SEQ ID NO: 653 - DNA, 20 bp, synthetic construct
  misc_feature 1..20: note = B2M targeting sequences
  source 1..20: mol_type = other DNA
ttcctgaattgctatgtgtc

SEQ ID NO: 654 - DNA, 20 bp, synthetic construct
  misc_feature 1..20: note = B2M targeting sequences
  source 1..20: mol_type = other DNA
ctcaggtactccaaagattc

SEQ ID NO: 655 - DNA, 20 bp, synthetic construct
  misc_feature 1..20: note = B2M targeting sequences
  source 1..20: mol_type = other DNA
cttaccccacttaactatct

SEQ ID NO: 656 - DNA, 20 bp, synthetic construct
  misc_feature 1..20: note = B2M targeting sequences
  source 1..20: mol_type = other DNA
ctcgcgctactctctctttc

SEQ ID NO: 657 - DNA, 20 bp, synthetic construct
  misc_feature 1..20: note = B2M targeting sequences
  source 1..20: mol_type = other DNA
tcgatctatgaaaaagacag

SEQ ID NO: 658 - DNA, 20 bp, synthetic construct
  misc_feature 1..20: note = B2M targeting sequences
  source 1..20: mol_type = other DNA
gagacatgtaagcagcatca

SEQ ID NO: 659 - DNA, 20 bp, synthetic construct
  misc_feature 1..20: note = B2M targeting sequences
  source 1..20: mol_type = other DNA
acatgtaagcagcatcatgg

SEQ ID NO: 660 - DNA, 20 bp, synthetic construct
  misc_feature 1..20: note = B2M targeting sequences
  source 1..20: mol_type = other DNA
gaagtcctagaatgagcgcc

SEQ ID NO: 661 - DNA, 20 bp, synthetic construct
  misc_feature 1..20: note = B2M targeting sequences
  source 1..20: mol_type = other DNA
gagcgcccggtgtcccaagc

SEQ ID NO: 662 - DNA, 20 bp, synthetic construct
  misc_feature 1..20: note = B2M targeting sequences
  source 1..20: mol_type = other DNA
agcgcccggtgtcccaagct

SEQ ID NO: 663 - DNA, 20 bp, synthetic construct
  misc_feature 1..20: note = B2M targeting sequences
  source 1..20: mol_type = other DNA
gcgcccggtgtcccaagctg

SEQ ID NO: 664 - DNA, 20 bp, synthetic construct
  misc_feature 1..20: note = B2M targeting sequences
  source 1..20: mol_type = other DNA
ctggggcgcgcaccccagat

SEQ ID NO: 665 - DNA, 20 bp, synthetic construct
  misc_feature 1..20: note = B2M targeting sequences
  source 1..20: mol_type = other DNA
gggcgcgcaccccagatcgg

SEQ ID NO: 666 - DNA, 20 bp, synthetic construct
  misc_feature 1..20: note = B2M targeting sequences
  source 1..20: mol_type = other DNA
ggcgcgcaccccagatcgga

SEQ ID NO: 667 - DNA, 20 bp, synthetic construct
  misc_feature 1..20: note = B2M targeting sequences
  source 1..20: mol_type = other DNA
catcacgagactctaagaaa

SEQ ID NO: 668 - DNA, 20 bp, synthetic construct
  misc_feature 1..20: note = B2M targeting sequences
  source 1..20: mol_type = other DNA
taagaaaaggaaactgaaaa

SEQ ID NO: 669 - DNA, 20 bp, synthetic construct
  misc_feature 1..20: note = B2M targeting sequences
  source 1..20: mol_type = other DNA
aagaaaaggaaactgaaaac

SEQ ID NO: 670 - DNA, 20 bp, synthetic construct
  misc_feature 1..20: note = B2M targeting sequences
  source 1..20: mol_type = other DNA
gaaagtccctctctctaacc

SEQ ID NO: 671 - DNA, 20 bp, synthetic construct
  misc_feature 1..20: note = B2M targeting sequences
  source 1..20: mol_type = other DNA
ctaacctggcactgcgtcgc

SEQ ID NO: 672 - DNA, 20 bp, synthetic construct
  misc_feature 1..20: note = B2M targeting sequences
  source 1..20: mol_type = other DNA
ctggcactgcgtcgctggct

SEQ ID NO: 673 - DNA, 20 bp, synthetic construct
  misc_feature 1..20: note = B2M targeting sequences
  source 1..20: mol_type = other DNA
tgcgtcgctggcttggagac

SEQ ID NO: 674 - DNA, 20 bp, synthetic construct
  misc_feature 1..20: note = B2M targeting sequences
  source 1..20: mol_type = other DNA
gctggcttggagacaggtga

SEQ ID NO: 675 - DNA, 20 bp, synthetic construct
  misc_feature 1..20: note = B2M targeting sequences
  source 1..20: mol_type = other DNA
gagacaggtgacggtccctg

SEQ ID NO: 676 - DNA, 20 bp, synthetic construct
  misc_feature 1..20: note = B2M targeting sequences
  source 1..20: mol_type = other DNA
agacaggtgacggtccctgc

SEQ ID NO: 677 - DNA, 20 bp, synthetic construct
  misc_feature 1..20: note = B2M targeting sequences
  source 1..20: mol_type = other DNA
cctgcgggccttgtcctgat

SEQ ID NO: 678 - DNA, 20 bp, synthetic construct
  misc_feature 1..20: note = B2M targeting sequences
  source 1..20: mol_type = other DNA
cgggccttgtcctgattggc

SEQ ID NO: 679 - DNA, 20 bp, synthetic construct
  misc_feature 1..20: note = B2M targeting sequences
  source 1..20: mol_type = other DNA
gggccttgtcctgattggct

SEQ ID NO: 680 - DNA, 20 bp, synthetic construct
  misc_feature 1..20: note = B2M targeting sequences
  source 1..20: mol_type = other DNA
gggcacgcgtttaatataag

SEQ ID NO: 681 - DNA, 20 bp, synthetic construct
  misc_feature 1..20: note = B2M targeting sequences
  source 1..20: mol_type = other DNA
cacgcgtttaatataagtgg

SEQ ID NO: 682 - DNA, 20 bp, synthetic construct
  misc_feature 1..20: note = B2M targeting sequences
  source 1..20: mol_type = other DNA
tataagtggaggcgtcgcgc

SEQ ID NO: 683 - DNA, 20 bp, synthetic construct
  misc_feature 1..20: note = B2M targeting sequences
  source 1..20: mol_type = other DNA
aagtggaggcgtcgcgctgg

SEQ ID NO: 684 - DNA, 20 bp, synthetic construct
  misc_feature 1..20: note = B2M targeting sequences
  source 1..20: mol_type = other DNA
agtggaggcgtcgcgctggc

SEQ ID NO: 685 - DNA, 20 bp, synthetic construct
  misc_feature 1..20: note = B2M targeting sequences
  source 1..20: mol_type = other DNA
ttcctgaagctgacagcatt

SEQ ID NO: 686 - DNA, 20 bp, synthetic construct
  misc_feature 1..20: note = B2M targeting sequences
  source 1..20: mol_type = other DNA
tcctgaagctgacagcattc

SEQ ID NO: 687 - DNA, 20 bp, synthetic construct
  misc_feature 1..20: note = B2M targeting sequences
  source 1..20: mol_type = other DNA
gcccgaatgctgtcagcttc

SEQ ID NO: 688 - DNA, 20 bp, synthetic construct
  misc_feature 1..20: note = B2M targeting sequences
  source 1..20: mol_type = other DNA
aaacgcgtgcccagccaatc

SEQ ID NO: 689 - DNA, 20 bp, synthetic construct
  misc_feature 1..20: note = B2M targeting sequences
  source 1..20: mol_type = other DNA
gtgcccagccaatcaggaca

SEQ ID NO: 690 - DNA, 20 bp, synthetic construct
  misc_feature 1..20: note = B2M targeting sequences
  source 1..20: mol_type = other DNA
ccaatcaggacaaggcccgc

SEQ ID NO: 691 - DNA, 20 bp, synthetic construct
  misc_feature 1..20: note = B2M targeting sequences
  source 1..20: mol_type = other DNA
caatcaggacaaggcccgca

SEQ ID NO: 692 - DNA, 20 bp, synthetic construct
  misc_feature 1..20: note = B2M targeting sequences
  source 1..20: mol_type = other DNA
caagccagcgacgcagtgcc

SEQ ID NO: 693 - DNA, 20 bp, synthetic construct
  misc_feature 1..20: note = B2M targeting sequences
  source 1..20: mol_type = other DNA
cgcagtgccaggttagagag

SEQ ID NO: 694 - DNA, 20 bp, synthetic construct
  misc_feature 1..20: note = B2M targeting sequences
  source 1..20: mol_type = other DNA
gcagtgccaggttagagaga

SEQ ID NO: 695 - DNA, 20 bp, synthetic construct
  misc_feature 1..20: note = B2M targeting sequences
  source 1..20: mol_type = other DNA
gagtctcgtgatgtttaaga

SEQ ID NO: 696 - DNA, 20 bp, synthetic construct
  misc_feature 1..20: note = B2M targeting sequences
  source 1..20: mol_type = other DNA
taagaaggcatgcactagac

SEQ ID NO: 697 - DNA, 20 bp, synthetic construct
  misc_feature 1..20: note = B2M targeting sequences
  source 1..20: mol_type = other DNA
aagaaggcatgcactagact

SEQ ID NO: 698 - DNA, 20 bp, synthetic construct
  misc_feature 1..20: note = B2M targeting sequences
  source 1..20: mol_type = other DNA
tgagtttgctgtctgtacat

SEQ ID NO: 699 - DNA, 20 bp, synthetic construct
  misc_feature 1..20: note = B2M targeting sequences
  source 1..20: mol_type = other DNA
tacatcggcgccctccgatc

SEQ ID NO: 700 - DNA, 20 bp, synthetic construct
  misc_feature 1..20: note = B2M targeting sequences
  source 1..20: mol_type = other DNA
acatcggcgccctccgatct

SEQ ID NO: 701 - DNA, 20 bp, synthetic construct
  misc_feature 1..20: note = B2M targeting sequences
  source 1..20: mol_type = other DNA
catcggcgccctccgatctg

SEQ ID NO: 702 - DNA, 20 bp, synthetic construct
  misc_feature 1..20: note = B2M targeting sequences
  source 1..20: mol_type = other DNA
ctggggtgcgcgccccagct

SEQ ID NO: 703 - DNA, 20 bp, synthetic construct
  misc_feature 1..20: note = B2M targeting sequences
  source 1..20: mol_type = other DNA
tggggtgcgcgccccagctt

SEQ ID NO: 704 - DNA, 20 bp, synthetic construct
  misc_feature 1..20: note = B2M targeting sequences
  source 1..20: mol_type = other DNA
cgcgccccagcttgggacac

SEQ ID NO: 705 - DNA, 20 bp, synthetic construct
  misc_feature 1..20: note = B2M targeting sequences
  source 1..20: mol_type = other DNA
gcgccccagcttgggacacc

SEQ ID NO: 706 - DNA, 20 bp, synthetic construct
  misc_feature 1..20: note = B2M targeting sequences
  source 1..20: mol_type = other DNA
caagtcacttagcatctctg

SEQ ID NO: 707 - DNA, 20 bp, synthetic construct
  misc_feature 1..20: note = B2M targeting sequences
  source 1..20: mol_type = other DNA
acagaagttctccttctgct

SEQ ID NO: 708 - DNA, 20 bp, synthetic construct
  misc_feature 1..20: note = B2M targeting sequences
  source 1..20: mol_type = other DNA
attcaaagatcttaatcttc

SEQ ID NO: 709 - DNA, 20 bp, synthetic construct
  misc_feature 1..20: note = B2M targeting sequences
  source 1..20: mol_type = other DNA
ttcaaagatcttaatcttct

SEQ ID NO: 710 - DNA, 20 bp, synthetic construct
  misc_feature 1..20: note = B2M targeting sequences
  source 1..20: mol_type = other DNA
ttttctcgaatgaaaaatgc

SEQ ID NO: 711 - DNA, 20 bp, synthetic construct
  misc_feature 1..20: note = B2M targeting sequences
  source 1..20: mol_type = other DNA
tgcaggtccgagcagttaac

SEQ ID NO: 712 - DNA, 20 bp, synthetic construct
  misc_feature 1..20: note = B2M targeting sequences
  source 1..20: mol_type = other DNA
ggtccgagcagttaactggc

SEQ ID NO: 713 - DNA, 20 bp, synthetic construct
  misc_feature 1..20: note = B2M targeting sequences
  source 1..20: mol_type = other DNA
gtccgagcagttaactggct

SEQ ID NO: 714 - DNA, 20 bp, synthetic construct
  misc_feature 1..20: note = B2M targeting sequences
  source 1..20: mol_type = other DNA
tccgagcagttaactggctg

SEQ ID NO: 715 - DNA, 20 bp, synthetic construct
  misc_feature 1..20: note = B2M targeting sequences
  source 1..20: mol_type = other DNA
agcaagtcacttagcatctc

SEQ ID NO: 716 - DNA, 20 bp, synthetic construct
  misc_feature 1..20: note = B2M targeting sequences
  source 1..20: mol_type = other DNA
gcaagtcacttagcatctct

SEQ ID NO: 717 - DNA, 20 bp, synthetic construct
  misc_feature 1..20: note = B2M targeting sequences
  source 1..20: mol_type = other DNA
tggggccagtctgcaaagcg

SEQ ID NO: 718 - DNA, 20 bp, synthetic construct
  misc_feature 1..20: note = B2M targeting sequences
  source 1..20: mol_type = other DNA
ggggccagtctgcaaagcga

SEQ ID NO: 719 - DNA, 20 bp, synthetic construct
  misc_feature 1..20: note = B2M targeting sequences
  source 1..20: mol_type = other DNA
gggccagtctgcaaagcgag

SEQ ID NO: 720 - DNA, 20 bp, synthetic construct
  misc_feature 1..20: note = B2M targeting sequences
  source 1..20: mol_type = other DNA
ggccagtctgcaaagcgagg

SEQ ID NO: 721 - DNA, 20 bp, synthetic construct
  misc_feature 1..20: note = B2M targeting sequences
  source 1..20: mol_type = other DNA
ggacaccgggcgctcattct

SEQ ID NO: 722 - DNA, 20 bp, synthetic construct
  misc_feature 1..20: note = B2M targeting sequences
  source 1..20: mol_type = other DNA
ggcgctcattctaggacttc

SEQ ID NO: 723 - DNA, 20 bp, synthetic construct
  misc_feature 1..20: note = B2M targeting sequences
  source 1..20: mol_type = other DNA
ctcattctaggacttcaggc

SEQ ID NO: 724 - DNA, 20 bp, synthetic construct
  misc_feature 1..20: note = B2M targeting sequences
  source 1..20: mol_type = other DNA
attctaggacttcaggctgg

SEQ ID NO: 725 - DNA, 20 bp, synthetic construct
  misc_feature 1..20: note = B2M targeting sequences
  source 1..20: mol_type = other DNA
ttcaggctggaggcacatta

SEQ ID NO: 726 - DNA, 20 bp, synthetic construct
  misc_feature 1..20: note = B2M targeting sequences
  source 1..20: mol_type = other DNA
tgccccctcgctttgcagac

SEQ ID NO: 727 - DNA, 20 bp, synthetic construct
  misc_feature 1..20: note = B2M targeting sequences
  source 1..20: mol_type = other DNA
gatgctaagtgacttgctaa

SEQ ID NO: 728 - DNA, 20 bp, synthetic construct
  misc_feature 1..20: note = B2M targeting sequences
  source 1..20: mol_type = other DNA
gccccagccagttaactgct

SEQ ID NO: 729 - DNA, 20 bp, synthetic construct
  misc_feature 1..20: note = B2M targeting sequences
  source 1..20: mol_type = other DNA
gcatttttcattcgagaaaa

SEQ ID NO: 730 - DNA, 20 bp, synthetic construct
  misc_feature 1..20: note = B2M targeting sequences
  source 1..20: mol_type = other DNA
tttgaatgctacctagcaga

SEQ ID NO: 731 - DNA, 20 bp, synthetic construct
  misc_feature 1..20: note = B2M targeting sequences
  source 1..20: mol_type = other DNA
ttctgtttataactacagct

SEQ ID NO: 732 - DNA, 20 bp, synthetic construct
  misc_feature 1..20: note = B2M targeting sequences
  source 1..20: mol_type = other DNA
tctgtttataactacagctt

SEQ ID NO: 733 - DNA, 21 bp, synthetic construct
  misc_feature 1..21: note = B2M shRNA
  source 1..21: mol_type = other DNA
agagaatggaaagtcaaattt

SEQ ID NO: 734 - DNA, 21 bp, synthetic construct
  misc_feature 1..21: note = B2M shRNA
  source 1..21: mol_type = other DNA
atggacatgatcttctttata

SEQ ID NO: 735 - DNA, 21 bp, synthetic construct
  misc_feature 1..21: note = B2M shRNA
  source 1..21: mol_type = other DNA
tggacatgatcttctttataa

SEQ ID NO: 736 - DNA, 21 bp, synthetic construct
  misc_feature 1..21: note = B2M shRNA
  source 1..21: mol_type = other DNA
ggacatgatcttctttataat

SEQ ID NO: 737 - DNA, 21 bp, synthetic construct
  misc_feature 1..21: note = B2M shRNA
  source 1..21: mol_type = other DNA
tgacaggattattggaaattt

SEQ ID NO: 738 - DNA, 21 bp, synthetic construct
  misc_feature 1..21: note = B2M shRNA
  source 1..21: mol_type = other DNA
ttgtggttaatctggtttatt

SEQ ID NO: 739 - DNA, 21 bp, synthetic construct
  misc_feature 1..21: note = B2M shRNA
  source 1..21: mol_type = other DNA
tgtggttaatctggtttattt

SEQ ID NO: 740 - DNA, 21 bp, synthetic construct
  misc_feature 1..21: note = B2M shRNA
  source 1..21: mol_type = other DNA
gcagagaatggaaagtcaaat

SEQ ID NO: 741 - DNA, 21 bp, synthetic construct
  misc_feature 1..21: note = B2M shRNA
  source 1..21: mol_type = other DNA
cagagaatggaaagtcaaatt

SEQ ID NO: 742 - DNA, 21 bp, synthetic construct
  misc_feature 1..21: note = B2M shRNA
  source 1..21: mol_type = other DNA
gagaatggaaagtcaaatttc

SEQ ID NO: 743 - DNA, 21 bp, synthetic construct
  misc_feature 1..21: note = B2M shRNA
  source 1..21: mol_type = other DNA
gtcacagcccaagatagttaa

SEQ ID NO: 744 - DNA, 21 bp, synthetic construct
  misc_feature 1..21: note = B2M shRNA
  source 1..21: mol_type = other DNA
tgcttatacacttacacttta

SEQ ID NO: 745 - DNA, 21 bp, synthetic construct
  misc_feature 1..21: note = B2M shRNA
  source 1..21: mol_type = other DNA
gcttatacacttacactttat

SEQ ID NO: 746 - DNA, 21 bp, synthetic construct
  misc_feature 1..21: note = B2M shRNA
  source 1..21: mol_type = other DNA
cttatacacttacactttatg

SEQ ID NO: 747 - DNA, 21 bp, synthetic construct
  misc_feature 1..21: note = B2M shRNA
  source 1..21: mol_type = other DNA
acatggacatgatcttcttta

SEQ ID NO: 748 - DNA, 21 bp, synthetic construct
  misc_feature 1..21: note = B2M shRNA
  source 1..21: mol_type = other DNA
catggacatgatcttctttat

SEQ ID NO: 749 - DNA, 21 bp, synthetic construct
  misc_feature 1..21: note = B2M shRNA
  source 1..21: mol_type = other DNA
atcaacatcttggtcagattt

SEQ ID NO: 750 - DNA, 21 bp, synthetic construct
  misc_feature 1..21: note = B2M shRNA
  source 1..21: mol_type = other DNA
cttgcactcaaagcttgttaa

SEQ ID NO: 751 - DNA, 21 bp, synthetic construct
  misc_feature 1..21: note = B2M shRNA
  source 1..21: mol_type = other DNA
agttaagcgtgcataagttaa

SEQ ID NO: 752 - DNA, 21 bp, synthetic construct
  misc_feature 1..21: note = B2M shRNA
  source 1..21: mol_type = other DNA
gcataagttaacttccaattt

SEQ ID NO: 753 - DNA, 21 bp, synthetic construct
  misc_feature 1..21: note = B2M shRNA
  source 1..21: mol_type = other DNA
tacatactctgcttagaattt

SEQ ID NO: 754 - DNA, 21 bp, synthetic construct
  misc_feature 1..21: note = B2M shRNA
  source 1..21: mol_type = other DNA
acatactctgcttagaatttg

SEQ ID NO: 755 - DNA, 21 bp, synthetic construct
  misc_feature 1..21: note = B2M shRNA
  source 1..21: mol_type = other DNA
ttgacaggattattggaaatt

SEQ ID NO: 756 - DNA, 21 bp, synthetic construct
  misc_feature 1..21: note = B2M shRNA
  source 1..21: mol_type = other DNA
gacaggattattggaaatttg

SEQ ID NO: 757 - DNA, 21 bp, synthetic construct
  misc_feature 1..21: note = B2M shRNA
  source 1..21: mol_type = other DNA
taaggcatggttgtggttaat

SEQ ID NO: 758 - DNA, 21 bp, synthetic construct
  misc_feature 1..21: note = B2M shRNA
  source 1..21: mol_type = other DNA
gttgtggttaatctggtttat

SEQ ID NO: 759 - DNA, 21 bp, synthetic construct
  misc_feature 1..21: note = B2M shRNA
  source 1..21: mol_type = other DNA
gttccacaagttaaataaatc

SEQ ID NO: 760 - DNA, 21 bp, synthetic construct
  misc_feature 1..21: note = B2M shRNA
  source 1..21: mol_type = other DNA
tccagcgtactccaaagattc

SEQ ID NO: 761 - DNA, 21 bp, synthetic construct
  misc_feature 1..21: note = B2M shRNA
  source 1..21: mol_type = other DNA
tactccaaagattcaggttta

SEQ ID NO: 762 - DNA, 21 bp, synthetic construct
  misc_feature 1..21: note = B2M shRNA
  source 1..21: mol_type = other DNA
actccaaagattcaggtttac

SEQ ID NO: 763 - DNA, 21 bp, synthetic construct
  misc_feature 1..21: note = B2M shRNA
  source 1..21: mol_type = other DNA
cacgtcatccagcagagaatg

SEQ ID NO: 764 - DNA, 21 bp, synthetic construct
  misc_feature 1..21: note = B2M shRNA
  source 1..21: mol_type = other DNA
ggtttcatccatccgacattg

SEQ ID NO: 765 - DNA, 21 bp, synthetic construct
  misc_feature 1..21: note = B2M shRNA
  source 1..21: mol_type = other DNA
ccgacattgaagttgacttac

SEQ ID NO: 766 - DNA, 21 bp, synthetic construct
  misc_feature 1..21: note = B2M shRNA
  source 1..21: mol_type = other DNA
tgaagaatggagagagaattg

SEQ ID NO: 767 - DNA, 21 bp, synthetic construct
  misc_feature 1..21: note = B2M shRNA
  source 1..21: mol_type = other DNA
gagcattcagacttgtctttc

SEQ ID NO: 768 - DNA, 21 bp, synthetic construct
  misc_feature 1..21: note = B2M shRNA
  source 1..21: mol_type = other DNA
ttcagcaaggactggtctttc

SEQ ID NO: 769 - DNA, 21 bp, synthetic construct
  misc_feature 1..21: note = B2M shRNA
  source 1..21: mol_type = other DNA
gcaaggactggtctttctatc

SEQ ID NO: 770 - DNA, 21 bp, synthetic construct
  misc_feature 1..21: note = B2M shRNA
  source 1..21: mol_type = other DNA
cgtgtgaaccatgtgactttg

SEQ ID NO: 771 - DNA, 21 bp, synthetic construct
  misc_feature 1..21: note = B2M shRNA
  source 1..21: mol_type = other DNA
ctttgtcacagcccaagatag

SEQ ID NO: 772 - DNA, 21 bp, synthetic construct
  misc_feature 1..21: note = B2M shRNA
  source 1..21: mol_type = other DNA
tcacagcccaagatagttaag

SEQ ID NO: 773 - DNA, 21 bp, synthetic construct
  misc_feature 1..21: note = B2M shRNA
  source 1..21: mol_type = other DNA
agtgggatcgagacatgtaag

SEQ ID NO: 774 - DNA, 21 bp, synthetic construct
  misc_feature 1..21: note = B2M shRNA
  source 1..21: mol_type = other DNA
aggtttgaagatgccgcattt

SEQ ID NO: 775 - DNA, 21 bp, synthetic construct
  misc_feature 1..21: note = B2M shRNA
  source 1..21: mol_type = other DNA
ggtttgaagatgccgcatttg

SEQ ID NO: 776 - DNA, 21 bp, synthetic construct
  misc_feature 1..21: note = B2M shRNA
  source 1..21: mol_type = other DNA
ttgatatgcttatacacttac

SEQ ID NO: 777 - DNA, 21 bp, synthetic construct
  misc_feature 1..21: note = B2M shRNA
  source 1..21: mol_type = other DNA
tgagtgctgtctccatgtttg

SEQ ID NO: 778 - DNA, 21 bp, synthetic construct
  misc_feature 1..21: note = B2M shRNA
  source 1..21: mol_type = other DNA
tgtctccatgtttgatgtatc

SEQ ID NO: 779 - DNA, 21 bp, synthetic construct
  misc_feature 1..21: note = B2M shRNA
  source 1..21: mol_type = other DNA
tcaacatcttggtcagatttg

SEQ ID NO: 780 - DNA, 21 bp, synthetic construct
  misc_feature 1..21: note = B2M shRNA
  source 1..21: mol_type = other DNA
tcagatttgaactcttcaatc

SEQ ID NO: 781 - DNA, 21 bp, synthetic construct
  misc_feature 1..21: note = B2M shRNA
  source 1..21: mol_type = other DNA
ttcaatctcttgcactcaaag

SEQ ID NO: 782 - DNA, 21 bp, synthetic construct
  misc_feature 1..21: note = B2M shRNA
  source 1..21: mol_type = other DNA
ttgcactcaaagcttgttaag

SEQ ID NO: 783 - DNA, 21 bp, synthetic construct
  misc_feature 1..21: note = B2M shRNA
  source 1..21: mol_type = other DNA
actcaaagcttgttaagatag

SEQ ID NO: 784 - DNA, 21 bp, synthetic construct
  misc_feature 1..21: note = B2M shRNA
  source 1..21: mol_type = other DNA
agatagttaagcgtgcataag

SEQ ID NO: 785 - DNA, 21 bp, synthetic construct
  misc_feature 1..21: note = B2M shRNA
  source 1..21: mol_type = other DNA
tgcataagttaacttccaatt

SEQ ID NO: 786 - DNA, 21 bp, synthetic construct
  misc_feature 1..21: note = B2M shRNA
  source 1..21: mol_type = other DNA
gttaacttccaatttacatac

SEQ ID NO: 787 - DNA, 21 bp, synthetic construct
  misc_feature 1..21: note = B2M shRNA
  source 1..21: mol_type = other DNA
attgacaggattattggaaat

SEQ ID NO: 788 - DNA, 21 bp, synthetic construct
  misc_feature 1..21: note = B2M shRNA
  source 1..21: mol_type = other DNA
gtaaggcatggttgtggttaa

SEQ ID NO: 789 - DNA, 21 bp, synthetic construct
  misc_feature 1..21: note = B2M shRNA
  source 1..21: mol_type = other DNA
ggttgtggttaatctggttta

SEQ ID NO: 790 - DNA, 21 bp, synthetic construct
  misc_feature 1..21: note = B2M shRNA
  source 1..21: mol_type = other DNA
ttcctgaagctgacagcattc

SEQ ID NO: 791 - DNA, 21 bp, synthetic construct
  misc_feature 1..21: note = B2M shRNA
  source 1..21: mol_type = other DNA
gctatccagcgtactccaaag

SEQ ID NO: 792 - DNA, 21 bp, synthetic construct
  misc_feature 1..21: note = B2M shRNA
  source 1..21: mol_type = other DNA
catccagcagagaatggaaag

SEQ ID NO: 793 - DNA, 21 bp, synthetic construct
  misc_feature 1..21: note = B2M shRNA
  source 1..21: mol_type = other DNA
caaatttcctgaattgctatg

SEQ ID NO: 794 - DNA, 21 bp, synthetic construct
  misc_feature 1..21: note = B2M shRNA
  source 1..21: mol_type = other DNA
attgctatgtgtctgggtttc

SEQ ID NO: 795 - DNA, 21 bp, synthetic construct
  misc_feature 1..21: note = B2M shRNA
  source 1..21: mol_type = other DNA
gaagatgccgcatttggattg

SEQ ID NO: 796 - DNA, 21 bp, synthetic construct
  misc_feature 1..21: note = B2M shRNA
  source 1..21: mol_type = other DNA
caatttacatactctgcttag

SEQ ID NO: 797 - DNA, 21 bp, synthetic construct
  misc_feature 1..21: note = B2M shRNA
  source 1..21: mol_type = other DNA
tatccagcgtactccaaagat

SEQ ID NO: 798 - DNA, 21 bp, synthetic construct
  misc_feature 1..21: note = B2M shRNA
  source 1..21: mol_type = other DNA
atccagcgtactccaaagatt

SEQ ID NO: 799 - DNA, 21 bp, synthetic construct
  misc_feature 1..21: note = B2M shRNA
  source 1..21: mol_type = other DNA
ctccaaagattcaggtttact

SEQ ID NO: 800 - DNA, 21 bp, synthetic construct
  misc_feature 1..21: note = B2M shRNA
  source 1..21: mol_type = other DNA
tgctatgtgtctgggtttcat

SEQ ID NO: 801 - DNA, 21 bp, synthetic construct
  misc_feature 1..21: note = B2M shRNA
  source 1..21: mol_type = other DNA
tttcatccatccgacattgaa

SEQ ID NO: 802 - DNA, 21 bp, synthetic construct
  misc_feature 1..21: note = B2M shRNA
  source 1..21: mol_type = other DNA
gaagttgacttactgaagaat

SEQ ID NO: 803 - DNA, 21 bp, synthetic construct
  misc_feature 1..21: note = B2M shRNA
  source 1..21: mol_type = other DNA
gaagaatggagagagaattga

SEQ ID NO: 804 - DNA, 21 bp, synthetic construct
  misc_feature 1..21: note = B2M shRNA
  source 1..21: mol_type = other DNA
agaatggagagagaattgaaa

SEQ ID NO: 805 - DNA, 21 bp, synthetic construct
  misc_feature 1..21: note = B2M shRNA
  source 1..21: mol_type = other DNA
cagcaaggactggtctttcta

SEQ ID NO: 806 - DNA, 21 bp, synthetic construct
  misc_feature 1..21: note = B2M shRNA
  source 1..21: mol_type = other DNA
agcaaggactggtctttctat

SEQ ID NO: 807 - DNA, 21 bp, synthetic construct
  misc_feature 1..21: note = B2M shRNA
  source 1..21: mol_type = other DNA
actttgtcacagcccaagata

SEQ ID NO: 808 - DNA, 21 bp, synthetic construct
  misc_feature 1..21: note = B2M shRNA
  source 1..21: mol_type = other DNA
ttgtcacagcccaagatagtt

SEQ ID NO: 809 - DNA, 21 bp, synthetic construct
  misc_feature 1..21: note = B2M shRNA
  source 1..21: mol_type = other DNA
tgtcacagcccaagatagtta

SEQ ID NO: 810 - DNA, 21 bp, synthetic construct
  misc_feature 1..21: note = B2M shRNA
  source 1..21: mol_type = other DNA
cacagcccaagatagttaagt

SEQ ID NO: 811 - DNA, 21 bp, synthetic construct
  misc_feature 1..21: note = B2M shRNA
  source 1..21: mol_type = other DNA
gcagcatcatggaggtttgaa

SEQ ID NO: 812 - DNA, 21 bp, synthetic construct
  misc_feature 1..21: note = B2M shRNA
  source 1..21: mol_type = other DNA
ccgcatttggattggatgaat

SEQ ID NO: 813 - DNA, 21 bp, synthetic construct
  misc_feature 1..21: note = B2M shRNA
  source 1..21: mol_type = other DNA
ttgagtgctgtctccatgttt

SEQ ID NO: 814 - DNA, 21 bp, synthetic construct
  misc_feature 1..21: note = B2M shRNA
  source 1..21: mol_type = other DNA
agtgctgtctccatgtttgat

SEQ ID NO: 815 - DNA, 21 bp, synthetic construct
  misc_feature 1..21: note = B2M shRNA
  source 1..21: mol_type = other DNA
ctgtctccatgtttgatgtat

SEQ ID NO: 816 - DNA, 21 bp, synthetic construct
  misc_feature 1..21: note = B2M shRNA
  source 1..21: mol_type = other DNA
tctaggagggctggcaactta

SEQ ID NO: 817 - DNA, 21 bp, synthetic construct
  misc_feature 1..21: note = B2M shRNA
  source 1..21: mol_type = other DNA
caacatcttggtcagatttga

SEQ ID NO: 818 - DNA, 21 bp, synthetic construct
  misc_feature 1..21: note = B2M shRNA
  source 1..21: mol_type = other DNA
gtcagatttgaactcttcaat

SEQ ID NO: 819 - DNA, 21 bp, synthetic construct
  misc_feature 1..21: note = B2M shRNA
  source 1..21: mol_type = other DNA
tcttgcactcaaagcttgtta

SEQ ID NO: 820 - DNA, 21 bp, synthetic construct
  misc_feature 1..21: note = B2M shRNA
  source 1..21: mol_type = other DNA
tgcactcaaagcttgttaaga

SEQ ID NO: 821 - DNA, 21 bp, synthetic construct
  misc_feature 1..21: note = B2M shRNA
  source 1..21: mol_type = other DNA
gcactcaaagcttgttaagat

SEQ ID NO: 822 - DNA, 21 bp, synthetic construct
  misc_feature 1..21: note = B2M shRNA
  source 1..21: mol_type = other DNA
cactcaaagcttgttaagata

SEQ ID NO: 823 - DNA, 21 bp, synthetic construct
  misc_feature 1..21: note = B2M shRNA
  source 1..21: mol_type = other DNA
tcaaagcttgttaagatagtt

SEQ ID NO: 824 - DNA, 21 bp, synthetic construct
  misc_feature 1..21: note = B2M shRNA
  source 1..21: mol_type = other DNA
caaagcttgttaagatagtta

SEQ ID NO: 825 - DNA, 21 bp, synthetic construct
  misc_feature 1..21: note = B2M shRNA
  source 1..21: mol_type = other DNA
gatagttaagcgtgcataagt

SEQ ID NO: 826 - DNA, 21 bp, synthetic construct
  misc_feature 1..21: note = B2M shRNA
  source 1..21: mol_type = other DNA
atagttaagcgtgcataagtt

SEQ ID NO: 827 - DNA, 21 bp, synthetic construct
  misc_feature 1..21: note = B2M shRNA
  source 1..21: mol_type = other DNA
tagttaagcgtgcataagtta

SEQ ID NO: 828 - DNA, 21 bp, synthetic construct
  misc_feature 1..21: note = B2M shRNA
  source 1..21: mol_type = other DNA
ttaagcgtgcataagttaact

SEQ ID NO: 829 - DNA, 21 bp, synthetic construct
  misc_feature 1..21: note = B2M shRNA
  source 1..21: mol_type = other DNA
taagcgtgcataagttaactt

SEQ ID NO: 830 - DNA, 21 bp, synthetic construct
  misc_feature 1..21: note = B2M shRNA
  source 1..21: mol_type = other DNA
atttacatactctgcttagaa

SEQ ID NO: 831 - DNA, 21 bp, synthetic construct
  misc_feature 1..21: note = B2M shRNA
  source 1..21: mol_type = other DNA
tttacatactctgcttagaat

SEQ ID NO: 832 - DNA, 21 bp, synthetic construct
  misc_feature 1..21: note = B2M shRNA
  source 1..21: mol_type = other DNA
acaggattattggaaatttgt

SEQ ID NO: 833 - DNA, 21 bp, synthetic construct
  misc_feature 1..21: note = B2M shRNA
  source 1..21: mol_type = other DNA
caggattattggaaatttgtt

SEQ ID NO: 834 - DNA, 21 bp, synthetic construct
  misc_feature 1..21: note = B2M shRNA
  source 1..21: mol_type = other DNA
aggcatggttgtggttaatct

SEQ ID NO: 835 - DNA, 21 bp, synthetic construct
  misc_feature 1..21: note = B2M shRNA
  source 1..21: mol_type = other DNA
cagcagagaatggaaagtcaa

SEQ ID NO: 836 - DNA, 21 bp, synthetic construct
  misc_feature 1..21: note = B2M shRNA
  source 1..21: mol_type = other DNA
tccgacattgaagttgactta

SEQ ID NO: 837 - DNA, 21 bp, synthetic construct
  misc_feature 1..21: note = B2M shRNA
  source 1..21: mol_type = other DNA
ctggtctttctatctcttgta

SEQ ID NO: 838 - DNA, 21 bp, synthetic construct
  misc_feature 1..21: note = B2M shRNA
  source 1..21: mol_type = other DNA
ccgtgtgaaccatgtgacttt

SEQ ID NO: 839 - DNA, 21 bp, synthetic construct
  misc_feature 1..21: note = B2M shRNA
  source 1..21: mol_type = other DNA
cccaagatagttaagtgggat

SEQ ID NO: 840 - DNA, 21 bp, synthetic construct
  misc_feature 1..21: note = B2M shRNA
  source 1..21: mol_type = other DNA
ggttgctccacaggtagctct

SEQ ID NO: 841 - DNA, 21 bp, synthetic construct
  misc_feature 1..21: note = B2M shRNA
  source 1..21: mol_type = other DNA
gctccacaggtagctctagga

SEQ ID NO: 842 - DNA, 21 bp, synthetic construct
  misc_feature 1..21: note = B2M shRNA
  source 1..21: mol_type = other DNA
gggagcagagaattctcttat

SEQ ID NO: 843 - DNA, 21 bp, synthetic construct
  misc_feature 1..21: note = B2M shRNA
  source 1..21: mol_type = other DNA
ggagcagagaattctcttatc

SEQ ID NO: 844 - DNA, 21 bp, synthetic construct
  misc_feature 1..21: note = B2M shRNA
  source 1..21: mol_type = other DNA
gagcagagaattctcttatcc

SEQ ID NO: 845 - DNA, 21 bp, synthetic construct
  misc_feature 1..21: note = B2M shRNA
  source 1..21: mol_type = other DNA
gagaattctcttatccaacat

SEQ ID NO: 846 - DNA, 21 bp, synthetic construct
  misc_feature 1..21: note = B2M shRNA
  source 1..21: mol_type = other DNA
gaattctcttatccaacatca

SEQ ID NO: 847 - DNA, 21 bp, synthetic construct
  misc_feature 1..21: note = B2M shRNA
  source 1..21: mol_type = other DNA
aagtggagcattcagacttgt

SEQ ID NO: 848 - DNA, 21 bp, synthetic construct
  misc_feature 1..21: note = B2M shRNA
  source 1..21: mol_type = other DNA
aaggactggtctttctatctc

SEQ ID NO: 849 - DNA, 21 bp, synthetic construct
  misc_feature 1..21: note = B2M shRNA
  source 1..21: mol_type = other DNA
aagcttgttaagatagttaag

SEQ ID NO: 850 - DNA, 21 bp, synthetic construct
  misc_feature 1..21: note = B2M shRNA
  source 1..21: mol_type = other DNA
aagcgtgcataagttaacttc

SEQ ID NO: 851 - DNA, 21 bp, synthetic construct
  misc_feature 1..21: note = B2M shRNA
  source 1..21: mol_type = other DNA
aagatgccgcatttggattgg

SEQ ID NO: 852 - DNA, 21 bp, synthetic construct
  misc_feature 1..21: note = B2M shRNA
  source 1..21: mol_type = other DNA
aagaatggagagagaattgaa

SEQ ID NO: 853 - DNA, 21 bp, synthetic construct
  misc_feature 1..21: note = B2M shRNA
  source 1..21: mol_type = other DNA
aacatcaacatcttggtcaga

SEQ ID NO: 854 - DNA, 21 bp, synthetic construct
  misc_feature 1..21: note = B2M shRNA
  source 1..21: mol_type = other DNA
aaggcatggttgtggttaatc

SEQ ID NO: 855 - DNA, 21 bp, synthetic construct
  misc_feature 1..21: note = B2M shRNA
  source 1..21: mol_type = other DNA
aagcagcatcatggaggtttg

SEQ ID NO: 856 - DNA, 21 bp, synthetic construct
  misc_feature 1..21: note = B2M shRNA
  source 1..21: mol_type = other DNA
aagatgagtatgcctgccgtg

SEQ ID NO: 857 - DNA, 21 bp, synthetic construct
  misc_feature 1..21: note = B2M shRNA
  source 1..21: mol_type = other DNA
aagttgacttactgaagaatg

SEQ ID NO: 858 - DNA, 21 bp, synthetic construct
  misc_feature 1..21: note = B2M shRNA
  source 1..21: mol_type = other DNA
aagatagttaagcgtgcataa

SEQ ID NO: 859 - DNA, 21 bp, synthetic construct
  misc_feature 1..21: note = B2M shRNA
  source 1..21: mol_type = other DNA
aacttccaatttacatactct

SEQ ID NO: 860 - DNA, 21 bp, synthetic construct
  misc_feature 1..21: note = B2M shRNA
  source 1..21: mol_type = other DNA
aacatcttggtcagatttgaa

SEQ ID NO: 861 - DNA, 21 bp, synthetic construct
  misc_feature 1..21: note = B2M shRNA
  source 1..21: mol_type = other DNA
aactcttcaatctcttgcact

SEQ ID NO: 862 - DNA, 21 bp, synthetic construct
  misc_feature 1..21: note = B2M shRNA
  source 1..21: mol_type = other DNA
aatttcctgaattgctatgtg

SEQ ID NO: 863 - DNA, 21 bp, synthetic construct
  misc_feature 1..21: note = B2M shRNA
  source 1..21: mol_type = other DNA
aatggaaagtcaaatttcctg

SEQ ID NO: 864 - DNA, 21 bp, synthetic construct
  misc_feature 1..21: note = B2M shRNA
  source 1..21: mol_type = other DNA
aaccatgtgactttgtcacag

SEQ ID NO: 865 - DNA, 21 bp, synthetic construct
  misc_feature 1..21: note = B2M shRNA
  source 1..21: mol_type = other DNA
aattgacaggattattggaaa

SEQ ID NO: 866 - DNA, 21 bp, synthetic construct
  misc_feature 1..21: note = B2M shRNA
  source 1..21: mol_type = other DNA
aattctcttatccaacatcaa

SEQ ID NO: 867 - DNA, 21 bp, synthetic construct
  misc_feature 1..21: note = B2M shRNA
  source 1..21: mol_type = other DNA
aaagtggagcattcagacttg

SEQ ID NO: 868 - DNA, 21 bp, synthetic construct
  misc_feature 1..21: note = B2M shRNA
  source 1..21: mol_type = other DNA
aaagtcaaatttcctgaattg

SEQ ID NO: 869 - DNA, 21 bp, synthetic construct
  misc_feature 1..21: note = B2M shRNA
  source 1..21: mol_type = other DNA
gttgctccacaggtagctcta

SEQ ID NO: 870 - DNA, 21 bp, synthetic construct
  misc_feature 1..21: note = B2M shRNA
  source 1..21: mol_type = other DNA
aatttacatactctgcttaga

SEQ ID NO: 871 - DNA, 46 bp, synthetic construct
  misc_feature 1..46: note = B2M shRNA
  source 1..46: mol_type = other DNA
gcactcaaagcttgttaagatcgaaatcttaacaagctttgagtgc

SEQ ID NO: 872 - DNA, 46 bp, synthetic construct
  misc_feature 1..46: note = B2M shRNA
  source 1..46: mol_type = other DNA
gttaacttccaatttacataccgaagtatgtaaattggaagttaac

SEQ ID NO: 873 - DNA, 21 bp, synthetic construct
  misc_feature 1..21: note = HLA-A shRNA
  source 1..21: mol_type = other DNA
cttcttccttccctattaaaa

SEQ ID NO: 874 - DNA, 21 bp, synthetic construct
  misc_feature 1..21: note = HLA-A shRNA
  source 1..21: mol_type = other DNA
tctcactccatgaggtatttc

SEQ ID NO: 875 - DNA, 21 bp, synthetic construct
  misc_feature 1..21: note = HLA-A shRNA
  source 1..21: mol_type = other DNA
ctctcactccatgaggtattt

SEQ ID NO: 876 - DNA, 21 bp, synthetic construct
  misc_feature 1..21: note = HLA-A shRNA
  source 1..21: mol_type = other DNA
gaggaggaagagctcagatag

SEQ ID NO: 877 - DNA, 21 bp, synthetic construct
  misc_feature 1..21: note = HLA-A shRNA
  source 1..21: mol_type = other DNA
gctctcactccatgaggtatt

SEQ ID NO: 878 - DNA, 21 bp, synthetic construct
  misc_feature 1..21: note = HLA-A shRNA
  source 1..21: mol_type = other DNA
aggattacatcgccctgaaag

SEQ ID NO: 879 - DNA, 21 bp, synthetic construct
  misc_feature 1..21: note = HLA-A shRNA
  source 1..21: mol_type = other DNA
acaccgtccagaggatgtatg

SEQ ID NO: 880 - DNA, 21 bp, synthetic construct
  misc_feature 1..21: note = HLA-A shRNA
  source 1..21: mol_type = other DNA
agggtccttcttcctggatac

SEQ ID NO: 881 - DNA, 21 bp, synthetic construct
  misc_feature 1..21: note = HLA-A shRNA
  source 1..21: mol_type = other DNA
cctacgacggcaaggattaca

SEQ ID NO: 882 - DNA, 21 bp, synthetic construct
  misc_feature 1..21: note = HLA-A shRNA
  source 1..21: mol_type = other DNA
tcactccatgaggtatttctt

SEQ ID NO: 883 - DNA, 21 bp, synthetic construct
  misc_feature 1..21: note = HLA-A shRNA
  source 1..21: mol_type = other DNA
ctacgacggcaaggattacat

SEQ ID NO: 884 - DNA, 21 bp, synthetic construct
  misc_feature 1..21: note = HLA-A shRNA
  source 1..21: mol_type = other DNA
ctcactccatgaggtatttct

SEQ ID NO: 885 - DNA, 21 bp, synthetic construct
  misc_feature 1..21: note = HLA-A shRNA
  source 1..21: mol_type = other DNA
ggaggaagagctcagatagaa

SEQ ID NO: 886 - DNA, 21 bp, synthetic construct
  misc_feature 1..21: note = HLA-A shRNA
  source 1..21: mol_type = other DNA
cacaccgtccagaggatgtat

SEQ ID NO: 887 - DNA, 21 bp, synthetic construct
  misc_feature 1..21: note = HLA-A shRNA
  source 1..21: mol_type = other DNA
cacgctgtctctgaccatgaa

SEQ ID NO: 888 - DNA, 21 bp, synthetic construct
  misc_feature 1..21: note = HLA-A shRNA
  source 1..21: mol_type = other DNA
ctggacaggagcagagataca

SEQ ID NO: 889 - DNA, 21 bp, synthetic construct
  misc_feature 1..21: note = HLA-A shRNA
  source 1..21: mol_type = other DNA
tggaggaggaagagctcagat

SEQ ID NO: 890 - DNA, 21 bp, synthetic construct
  misc_feature 1..21: note = HLA-A shRNA
  source 1..21: mol_type = other DNA
ggctctcactccatgaggtat

SEQ ID NO: 891 - DNA, 21 bp, synthetic construct
  misc_feature 1..21: note = HLA-A shRNA
  source 1..21: mol_type = other DNA
catctctgtctcaacttcatg

SEQ ID NO: 892 - DNA, 21 bp, synthetic construct
  misc_feature 1..21: note = HLA-A shRNA
  source 1..21: mol_type = other DNA
tacgacggcaaggattacatc

SEQ ID NO: 893 - DNA, 21 bp, synthetic construct
  misc_feature 1..21: note = HLA-A shRNA
  source 1..21: mol_type = other DNA
ggattacatcgccctgaaaga

SEQ ID NO: 894 - DNA, 21 bp, synthetic construct
  misc_feature 1..21: note = HLA-A shRNA
  source 1..21: mol_type = other DNA
gattacatcgccctgaaagag

SEQ ID NO: 895 - DNA, 21 bp, synthetic construct
  misc_feature 1..21: note = HLA-A shRNA
  source 1..21: mol_type = other DNA
ctcagaccaccaagcacaagt

SEQ ID NO: 896 - DNA, 21 bp, synthetic construct
  misc_feature 1..21: note = HLA-A shRNA
  source 1..21: mol_type = other DNA
tcacaccgtccagaggatgta

SEQ ID NO: 897 - DNA, 21 bp, synthetic construct
  misc_feature 1..21: note = HLA-A shRNA
  source 1..21: mol_type = other DNA
actccatgaggtatttcttca

SEQ ID NO: 898 - DNA, 21 bp, synthetic construct
  misc_feature 1..21: note = HLA-A shRNA
  source 1..21: mol_type = other DNA
cactccatgaggtatttcttc

SEQ ID NO: 899 - DNA, 21 bp, synthetic construct
  misc_feature 1..21: note = HLA-A shRNA
  source 1..21: mol_type = other DNA
ccatgaggtatttcttcacat

SEQ ID NO: 900 - DNA, 21 bp, synthetic construct
  misc_feature 1..21: note = HLA-A shRNA
  source 1..21: mol_type = other DNA
acttcttccttccctattaaa

SEQ ID NO: 901 - DNA, 21 bp, synthetic construct
  misc_feature 1..21: note = HLA-A shRNA
  source 1..21: mol_type = other DNA
gtgtctctcacagcttgtaaa

SEQ ID NO: 902 - DNA, 21 bp, synthetic construct
  misc_feature 1..21: note = HLA-A shRNA
  source 1..21: mol_type = other DNA
ctgtgttcgtgtaggcataat

SEQ ID NO: 903 - DNA, 21 bp, synthetic construct
  misc_feature 1..21: note = HLA-A shRNA
  source 1..21: mol_type = other DNA
tgtgttcgtgtaggcataatg

SEQ ID NO: 904 - DNA, 21 bp, synthetic construct
  misc_feature 1..21: note = HLA-A shRNA
  source 1..21: mol_type = other DNA
taacttcttccttccctatta

SEQ ID NO: 905 - DNA, 21 bp, synthetic construct
  misc_feature 1..21: note = HLA-A shRNA
  source 1..21: mol_type = other DNA
tctggacaggagcagagatac

SEQ ID NO: 906 - DNA, 21 bp, synthetic construct
  misc_feature 1..21: note = HLA-A shRNA
  source 1..21: mol_type = other DNA
ttgctggcctggttctctttg

SEQ ID NO: 907 - DNA, 21 bp, synthetic construct
  misc_feature 1..21: note = HLA-A shRNA
  source 1..21: mol_type = other DNA
tgtctctcacagcttgtaaag

SEQ ID NO: 908 - DNA, 21 bp, synthetic construct
  misc_feature 1..21: note = HLA-A shRNA
  source 1..21: mol_type = other DNA
acttgaagaaccctgactttg

SEQ ID NO: 909 - DNA, 21 bp, synthetic construct
  misc_feature 1..21: note = HLA-A shRNA
  source 1..21: mol_type = other DNA
gaagaaccctgactttgtttc

SEQ ID NO: 910 - DNA, 21 bp, synthetic construct
  misc_feature 1..21: note = HLA-A shRNA
  source 1..21: mol_type = other DNA
tctgtgttcgtgtaggcataa

SEQ ID NO: 911 - DNA, 21 bp, synthetic construct
  misc_feature 1..21: note = HLA-A shRNA
  source 1..21: mol_type = other DNA
catggtgcactgagctgtaac

SEQ ID NO: 912 - DNA, 21 bp, synthetic construct
  misc_feature 1..21: note = HLA-A shRNA
  source 1..21: mol_type = other DNA
gtaacttcttccttccctatt

SEQ ID NO: 913 - DNA, 21 bp, synthetic construct
  misc_feature 1..21: note = HLA-A shRNA
  source 1..21: mol_type = other DNA
catgtgcagcatgagggtttg

SEQ ID NO: 914 - DNA, 21 bp, synthetic construct
  misc_feature 1..21: note = HLA-A shRNA
  source 1..21: mol_type = other DNA
ttgttcctgcccttccctttg

SEQ ID NO: 915 - DNA, 21 bp, synthetic construct
  misc_feature 1..21: note = HLA-A shRNA
  source 1..21: mol_type = other DNA
acccagttctcactcccattg

SEQ ID NO: 916 - DNA, 21 bp, synthetic construct
  misc_feature 1..21: note = HLA-A shRNA
  source 1..21: mol_type = other DNA
gggtttccagagaagccaatc

SEQ ID NO: 917 - DNA, 21 bp, synthetic construct
  misc_feature 1..21: note = HLA-A shRNA
  source 1..21: mol_type = other DNA
ttctccctctcccaacctatg

SEQ ID NO: 918 - DNA, 21 bp, synthetic construct
  misc_feature 1..21: note = HLA-A shRNA
  source 1..21: mol_type = other DNA
gtctctcacagcttgtaaagt

SEQ ID NO: 919 - DNA, 21 bp, synthetic construct
  misc_feature 1..21: note = HLA-A shRNA
  source 1..21: mol_type = other DNA
tgtgtctctcacagcttgtaa

SEQ ID NO: 920 - DNA, 21 bp, synthetic construct
  misc_feature 1..21: note = HLA-A shRNA
  source 1..21: mol_type = other DNA
gaggaagagctcagatagaaa

SEQ ID NO: 921 - DNA, 21 bp, synthetic construct
  misc_feature 1..21: note = HLA-A shRNA
  source 1..21: mol_type = other DNA
tgaagaaccctgactttgttt

SEQ ID NO: 922 - DNA, 21 bp, synthetic construct
  misc_feature 1..21: note = HLA-A shRNA
  source 1..21: mol_type = other DNA
ttgaagaaccctgactttgtt

SEQ ID NO: 923 - DNA, 21 bp, synthetic construct
  misc_feature 1..21: note = HLA-A shRNA
  source 1..21: mol_type = other DNA
gtgttcgtgtaggcataatgt

SEQ ID NO: 924 - DNA, 21 bp, synthetic construct
  misc_feature 1..21: note = HLA-A shRNA
  source 1..21: mol_type = other DNA
tggtgcactgagctgtaactt

SEQ ID NO: 925 - DNA, 21 bp, synthetic construct
  misc_feature 1..21: note = HLA-A shRNA
  source 1..21: mol_type = other DNA
ctccctctcccaacctatgta

SEQ ID NO: 926 - DNA, 21 bp, synthetic construct
  misc_feature 1..21: note = HLA-A shRNA
  source 1..21: mol_type = other DNA
aggaggaagagctcagataga

SEQ ID NO: 927 - DNA, 21 bp, synthetic construct
  misc_feature 1..21: note = HLA-A shRNA
  source 1..21: mol_type = other DNA
acctatgtagggtccttcttc

SEQ ID NO: 928 - DNA, 21 bp, synthetic construct
  misc_feature 1..21: note = HLA-A shRNA
  source 1..21: mol_type = other DNA
gggtccttcttcctggatact

SEQ ID NO: 929 - DNA, 21 bp, synthetic construct
  misc_feature 1..21: note = HLA-A shRNA
  source 1..21: mol_type = other DNA
ggtccttcttcctggatactc

SEQ ID NO: 930 - DNA, 21 bp, synthetic construct
  misc_feature 1..21: note = HLA-A shRNA
  source 1..21: mol_type = other DNA
gtccttcttcctggatactca

SEQ ID NO: 931 - DNA, 21 bp, synthetic construct
  misc_feature 1..21: note = HLA-A shRNA
  source 1..21: mol_type = other DNA
aagccaatcagtgtcgtcgcg

SEQ ID NO: 932 - DNA, 21 bp, synthetic construct
  misc_feature 1..21: note = HLA-A shRNA
  source 1..21: mol_type = other DNA
aagaggacctgcgctcttgga

SEQ ID NO: 933 - DNA, 21 bp, synthetic construct
  misc_feature 1..21: note = HLA-A shRNA
  source 1..21: mol_type = other DNA
aagtgtgagacagctgccttg

SEQ ID NO: 934 - DNA, 21 bp, synthetic construct
  misc_feature 1..21: note = HLA-A shRNA
  source 1..21: mol_type = other DNA
aaggcacctgcatgtgtctgt

SEQ ID NO: 935 - DNA, 21 bp, synthetic construct
  misc_feature 1..21: note = HLA-A shRNA
  source 1..21: mol_type = other DNA
aatcatctttcctgttccaga

SEQ ID NO: 936 - DNA, 21 bp, synthetic construct
  misc_feature 1..21: note = HLA-A shRNA
  source 1..21: mol_type = other DNA
aaaggcacctgcatgtgtctg

SEQ ID NO: 937 - DNA, 21 bp, synthetic construct
  misc_feature 1..21: note = HLA-A shRNA
  source 1..21: mol_type = other DNA
aaagaggacctgcgctcttgg

SEQ ID NO: 938 - DNA, 21 bp, synthetic construct
  misc_feature 1..21: note = HLA-A shRNA
  source 1..21: mol_type = other DNA
aaacgcatatgactcaccacg

SEQ ID NO: 939 - DNA, 19 bp, synthetic construct
  misc_feature 1..19: note = HLA-A shRNA
  source 1..19: mol_type = other DNA
ggaagagctcagatagaaa

SEQ ID NO: 940 - DNA, 19 bp, synthetic construct
  misc_feature 1..19: note = HLA-A shRNA
  source 1..19: mol_type = other DNA
gggagacacggaaagtgaa

SEQ ID NO: 941 - DNA, 21 bp, synthetic construct
  misc_feature 1..21: note = HLA-A shRNA
  source 1..21: mol_type = other DNA
cacctgccatgtgcagcatga

SEQ ID NO: 942 - DNA, 21 bp, synthetic construct
  misc_feature 1..21: note = HLA-A shRNA
  source 1..21: mol_type = other DNA
ggagatcacactgacctggca

SEQ ID NO: 943 - DNA, 20 bp, synthetic construct
  misc_feature 1..20: note = HLA-A shRNA
  source 1..20: mol_type = other DNA
ggattacatcgccctgaaag

SEQ ID NO: 944 - DNA, 20 bp, synthetic construct
  misc_feature 1..20: note = HLA-A shRNA
  source 1..20: mol_type = other DNA
gcaggagggtccggagtatt

SEQ ID NO: 945 - DNA, 20 bp, synthetic construct
  misc_feature 1..20: note = HLA-A shRNA
  source 1..20: mol_type = other DNA
ggacggggagacacggaaag

SEQ ID NO: 946 - DNA, 19 bp, synthetic construct
  misc_feature 1..19: note = HLA-A shRNA
  source 1..19: mol_type = other DNA
gaaagtgaaggcccactca

SEQ ID NO: 947 - DNA, 20 bp, synthetic construct
  misc_feature 1..20: note = HLA-A shRNA
  source 1..20: mol_type = other DNA
gatacctggagaacgggaag

SEQ ID NO: 948 - DNA, 20 bp, synthetic construct
  misc_feature 1..20: note = HLA-A shRNA
  source 1..20: mol_type = other DNA
gctgtggtggtgccttctgg

SEQ ID NO: 949 - DNA, 20 bp, synthetic construct
  misc_feature 1..20: note = HLA-A shRNA
  source 1..20: mol_type = other DNA
gctactacaaccagagcgag

SEQ ID NO: 950 - DNA, 19 bp, synthetic construct
  misc_feature 1..19: note = HLA-A shRNA
  source 1..19: mol_type = other DNA
gtggctccgcagatacctg

SEQ ID NO: 951 - DNA, 19 bp, synthetic construct
  misc_feature 1..19: note = HLA-A shRNA
  source 1..19: mol_type = other DNA
gccaatcagtgtcgtcgcg

SEQ ID NO: 952 - DNA, 19 bp, synthetic construct
  misc_feature 1..19: note = HLA-A shRNA
  source 1..19: mol_type = other DNA
gaggacctgcgctcttgga

SEQ ID NO: 953 - DNA, 19 bp, synthetic construct
  misc_feature 1..19: note = HLA-A shRNA
  source 1..19: mol_type = other DNA
gtgtgagacagctgccttg

SEQ ID NO: 954 - DNA, 19 bp, synthetic construct
  misc_feature 1..19: note = HLA-A shRNA
  source 1..19: mol_type = other DNA
ggcacctgcatgtgtctgt

SEQ ID NO: 955 - DNA, 19 bp, synthetic construct
  misc_feature 1..19: note = HLA-A shRNA
  source 1..19: mol_type = other DNA
tcatctttcctgttccaga

SEQ ID NO: 956 - DNA, 19 bp, synthetic construct
  misc_feature 1..19: note = HLA-A shRNA
  source 1..19: mol_type = other DNA
aggcacctgcatgtgtctg

SEQ ID NO: 957 - DNA, 19 bp, synthetic construct
  misc_feature 1..19: note = HLA-A shRNA
  source 1..19: mol_type = other DNA
agaggacctgcgctcttgg

SEQ ID NO: 958 - DNA, 19 bp, synthetic construct
  misc_feature 1..19: note = HLA-A shRNA
  source 1..19: mol_type = other DNA
acgcatatgactcaccacg

SEQ ID NO: 959 - RNA, 23 nt, synthetic construct
  misc_feature 1..23: note = shRNA flank
  source 1..23: mol_type = other RNA
acaccatgttgccagtctctagg

SEQ ID NO: 960 - RNA, 23 nt, synthetic construct
  misc_feature 1..23: note = shRNA flank
  source 1..23: mol_type = other RNA
tgatagcaatgtcagcagtgcct

SEQ ID NO: 961 - RNA, 23 nt, synthetic construct
  misc_feature 1..23: note = shRNA flank
  source 1..23: mol_type = other RNA
tattgctgttgacagtgagcgac

SEQ ID NO: 962 - RNA, 21 nt, synthetic construct
  misc_feature 1..21: note = shRNA flank
  source 1..21: mol_type = other RNA
tggcgtctggcccaaccacac

SEQ ID NO: 963 - RNA, 21 nt, synthetic construct
  misc_feature 1..21: note = shRNA flank
  source 1..21: mol_type = other RNA
gtaaggttgaccatactctac

SEQ ID NO: 964 - RNA, 12 nt, synthetic construct
  misc_feature 1..12: note = shRNA loop
  source 1..12: mol_type = other RNA
cttcctgtcaga

SEQ ID NO: 965 - RNA, 15 nt, synthetic construct
  misc_feature 1..15: note = shRNA loop
  source 1..15: mol_type = other RNA
cttccctttgtcaga

SEQ ID NO: 966 - RNA, 12 nt, synthetic construct
  misc_feature 1..12: note = shRNA loop
  source 1..12: mol_type = other RNA
gtgttattcttg

SEQ ID NO: 967 - RNA, 12 nt, synthetic construct
  misc_feature 1..12: note = shRNA loop
  source 1..12: mol_type = other RNA
gtgtcttaattg

SEQ ID NO: 968 - RNA, 12 nt, synthetic construct
  misc_feature 1..12: note = shRNA loop
  source 1..12: mol_type = other RNA
gtgttagtcttg

SEQ ID NO: 969 - skipped

SEQ ID NO: 970 - RNA, 12 nt, synthetic construct
  misc_feature 1..12: note = shRNA loop
  source 1..12: mol_type = other RNA
ggacatccaggg

SEQ ID NO: 971 - RNA, 15 nt, synthetic construct
  misc_feature 1..15: note = shRNA loop
  source 1..15: mol_type = other RNA
gtgaagccacagatg

SEQ ID NO: 972 - RNA, 10 nt, synthetic construct
  misc_feature 1..10: note = shRNA loop
  source 1..10: mol_type = other RNA
gattctaaaa

SEQ ID NO: 973 - protein, 23 aa, Homo sapiens
VIGFRILLLKVAGFNLLMTLRLW

SEQ ID NO: 974 - DNA, 69 bp, Homo sapiens; identical to SEQ ID NO: 271
  source 1..69: mol_type = unassigned DNA

SEQ ID NO: 975 - protein, 23 aa, Homo sapiens
TILYEILLGKATLYAVLVSALVL

SEQ ID NO: 976 - DNA, 69 bp, Homo sapiens; identical to SEQ ID NO: 273
  source 1..69: mol_type = unassigned DNA

SEQ ID NO: 977 - protein, 10 aa, Homo sapiens
MAMVKRKDSR

SEQ ID NO: 978 - DNA, 30 bp, Homo sapiens; identical to SEQ ID NO: 268
  source 1..30: mol_type = unassigned DNA

SEQ ID NO: 979 - DNA, 318 bp, synthetic construct
  misc_feature 1..318: note = HLA-E VL region
  source 1..318: mol_type = other DNA
cagatcgtgctgacccagagccccgccgtgatcagcgccagccccggcgagaaggtgatcctgacctgcagcgccagcagcagcgtgagctacatgcactggttccagcagaagcccggcaccagccccaagctgtggatctacggcaccagcaacctggccagcggcgtgcccgccaggttcagcggcggcggcagcggcaccagctacagcctgaccatcagcaggatggaggccgaggacgccgccacctactactgccagcagaggagcaggtaccccttcaccttcggcagcggcaccaagctggagatcaag

SEQ ID NO: 980 - DNA, 363 bp, synthetic construct
  misc_feature 1..363: note = HLA-E VH region
  source 1..363: mol_type = other DNA
caggtgcagctgagggagagcggccccagcctggtgaagcccagccagaccctgagcctgacctgcaccgtgagcggcttcagcctgaccagctacggcgtgcactgggtgaggcagccccccggcaagggcctggagtggctgggcgtgatctggaccggcggcaccaccaactacaacaccgccctgatgagcaggctgagcatcaccaaggacaacagcaagagccaggtgttcctgaagatgaacagcctgcagaccgacgacaccgccatctactactgcgccagggacggcgacagctacaacagggaggcctggttcgcctactggggccagggcaccctggtgaccgtgagcgcc

SEQ ID NO: 981 - protein, 247 aa, synthetic construct
  REGION 1..247: note = HLA-A02 scFv
MMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPRTSGGGTKLEIKGGGGSGGGGSGGGGSGGQVQLQQSGPELVKPGASVRISCKASGYTFTSYHIHWVKQRPGQGLEWIGWIYPGNVNTEYNEKFKGKATLTADKSSSTAYMHLSSLTSEDSAVYFCAREEITYAMDYWGQGTSVTVSSYG

SEQ ID NO: 982 - DNA, 20 bp, synthetic construct
  misc_feature 1..20: note = gRNA targeting sequence
  source 1..20: mol_type = other DNA
gagataatccttgccgtcgt

SEQ ID NO: 983 - protein, 516 aa, synthetic construct
  REGION 1..516: note = HLA-A03 inhibitory receptor
MDMRVPAQLLGLLLLWLRGARCEVKLEESGGGLVQPGGSMKLSCVASGFTFSNYWMNWVRQSPEKGLEWVAEIRLKSTNYATHYAESVKGRFTISRDDSKSSVYLQMNNLRAEDTGIYYCTTLITPDYWGQGTTLTVSSGGGGSGGGGSGGGGSGGDIVMTQSHKFMSTSVGDRVSITCKASQDVSTTVAWYQQKPGQSPKLLIYSASYRYTGVPDRFTGSGSGTDFTFTISSVQAEDLAVYYCQQHYSTPPTFGGGTKLEIKYGSQSSKPYLLTHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 984 - DNA, 1480 bp, synthetic construct
  misc_feature 1..1480: note = HLA-A03 inhibitory receptor
  source 1..1480: mol_type = other DNA
atggacatgagggtccccgctcagctcctggggctcctgctactctggctccgaggtgccagatgtgaagtgaagcttgaggagtctggaggaggcttggtgcaacctggaggatctatgaaactctcctgtgttgcctctggattcactttcagtaactactggatgaactgggtccgccagtctccagagaaggggcttgagtgggttgctgaaattagattgaaatctactaattatgcaacacattatgcggagtctgtgaaagggaggttcaccatctcaagagatgattccaaaagtagtgtctacctgcaaatgaacaacttaagagctgaagacactggcatttattactgtactactctgattacccctgactactggggccaaggcaccactctcacagtctcctcaggaggtggcggctctggaggcggaggtagcggaggtggaggctctggtggcgacattgtgatgacccagtctcacaaattcatgtccacatcagtaggagacagggtcagcatcacctgcaaggccagtcaggatgtgagtactactgtggcctggtatcagcagaaaccagggcaatctcctaaactactgatttattcggcatcctaccggtacactggagtccctgatcgcttcactggcagtggatctgggacggatttcactttcaccatcagcagtgtgcaggctgaagacctggcagtttattactgtcagcaacattatagtactcctcccacgttcggaggggggaccaagctggaaataaaatacggctcacagagctccaaaccctacctgctgactcaccccagtgaccccctggagctcgtggtctcaggaccgtctgggggccccagctccccgacaacaggccccacctccacatctggccctgaggaccagcccctcacccccaccgggtcggatccccagagtggtctgggaaggcacctgggggttgtgatcggcatcttggtggccgtcatcctactgctcctcctcctcctcctcctcttcctcatcctccgacatcgacgtcagggcaaacactggacatcgacccagagaaaggctgatttccaacatcctgcaggggctgtggggccagagcccacagacagaggcctgcagtggaggtccagcccagctgccgatgcccaggaagaaaacctctatgctgccgtgaagcacacacagcctgaggatggggtggagatggacactcggagcccacacgatgaagacccccaggcagtgacgtatgccgaggtgaaacactccagacctaggagagaaatggcctctcctccttccccactgtctggggaattcctggacacaaaggacagacaggcggaagaggacaggcagatggacactgaggctgctgcatctgaagccccccaggatgtgacctacgcccagctgcacagcttgaccctcagacgggaggcaactg

SEQ ID NO: 985 - protein, 522 aa, synthetic construct
  REGION 1..522: note = HLA-A0301 inhibitory receptor
MDMRVPAQLLGLLLLWLRGARCQVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARETPPSLGAFDIWGQGTMVTVSSGGGGSGGGGSGGGGSGGQSALTQPPSASGSPGQSVTISCTGTSSDVGGYNYVSWYQQHPGKAPKLMIYEVSKRPSGVPDRFSGSKSGNTASLTVSGLQAEDEADYYCSSYAGSNNWVFGGGTKLTVLYGSQSSKPYLLTHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 986 - DNA, 1566 bp, synthetic construct
  misc_feature 1..1566: note = HLA-A0301 inhibitory receptor
  source 1..1566: mol_type = other DNA
atggacatgagggtccccgctcagctcctggggctcctgctactctggctccgaggtgccagatgtcaggtgcagctggtgcagtctggcgccgaagtgaagaaacctggcgcctccgtgaaggtgtcctgcaaggccagcggctacaccttcaccagctacggcatcagctgggtccgccaggctcctggacagggactggaatggatgggctggatcagcgcctacaacggcaacaccaactacgcccagaaactgcagggcagagtgaccatgaccaccgacaccagcaccagcaccgcctacatggaacttcgaagcctgagaagcgacgacaccgccgtgtattactgtgcgagagaaacccccccctccctcggcgcttttgatatctggggccaagggacaatggtcaccgtgtcctcaggcggaggtggaagcggagggggaggatctggcggcggaggaagcggaggccagagcgccctgacacagcctcctagcgcgtctggcagccctggccagagcgtgaccatcagctgcaccggcaccagcagcgacgtgggcggctacaactacgtgtcctggtatcagcagcaccccggcaaggcccccaagctgatgatctacgaggtgtccaagcggcccagcggcgtgcccgatagattcagcggcagcaagagcggcaacaccgccagcctgaccgtgtctggactgcaggccgaggacgaggccgactactactgcagcagctatgcaggcagcaacaattgggtgttcggcggagggaccaagctgaccgtcctatacggctcacagagctccaaaccctacctgctgactcaccccagtgaccccctggagctcgtggtctcaggaccgtctgggggccccagctccccgacaacaggccccacctccacatctggccctgaggaccagcccctcacccccaccgggtcggatccccagagtggtctgggaaggcacctgggggttgtgatcggcatcttggtggccgtcatcctactgctcctcctcctcctcctcctcttcctcatcctccgacatcgacgtcagggcaaacactggacatcgacccagagaaaggctgatttccaacatcctgcaggggctgtggggccagagcccacagacagaggcctgcagtggaggtccagcccagctgccgatgcccaggaagaaaacctctatgctgccgtgaagcacacacagcctgaggatggggtggagatggacactcggagcccacacgatgaagacccccaggcagtgacgtatgccgaggtgaaacactccagacctaggagagaaatggcctctcctccttccccactgtctggggaattcctggacacaaaggacagacaggcggaagaggacaggcagatggacactgaggctgctgcatctgaagccccccaggatgtgacctacgcccagctgcacagcttgaccctcagacgggaggcaactgagcctcctccatcccaggaagggccctctccagctgtgcccagcatctacgccactctggccatccac

SEQ ID NO: 987 - protein, 518 aa, synthetic construct
  REGION 1..518: note = HLA-A11 inhibitory receptor
MDMRVPAQLLGLLLLWLRGARCEVQLVESGGGLVQPGGSLRLSCAASGFTFSSYWMHWVRQAPGKGLVWVSRINSDGSSTSYADSVKGRFTISRDNAKNTLYLQMNSLRAEDTAVYYCCLGVLLYNWFDPWGQGTLVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKYGSQSSKPYLLTHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 988 - DNA, 1554 bp, synthetic construct
  misc_feature 1..1554: note = HLA-A11 inhibitory receptor
  source 1..1554: mol_type = other DNA
atggacatgagggtccccgctcagctcctggggctcctgctactctggctccgaggtgccagatgtgaagtgcagctggtggaaagcggcggaggcctggtgcagcctggcggcagcctgagactgtcttgcgccgccagcggcttcaccttcagcagctactggatgcactgggtccgccaggcccctggcaagggactggtctgggtgtctcgaatcaacagcgacggcagcagcaccagctacgccgacagcgtgaagggccggttcaccatcagccgggacaacgccaagaacaccctgtacctgcagatgaacagcctgcgggccgaggacaccgccgtgtattactgttgtttgggtgttttattatacaactggttcgacccctggggccagggaaccctggtcaccgtgtcctcaggcggaggtggaagcggagggggaggatctggcggcggaggaagcggaggcgacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctcactttcggcggcggaacaaaggtggagatcaagtacggctcacagagctccaaaccctacctgctgactcaccccagtgaccccctggagctcgtggtctcaggaccgtctgggggccccagctccccgacaacaggccccacctccacatctggccctgaggaccagcccctcacccccaccgggtcggatccccagagtggtctgggaaggcacctgggggttgtgatcggcatcttggtggccgtcatcctactgctcctcctcctcctcctcctcttcctcatcctccgacatcgacgtcagggcaaacactggacatcgacccagagaaaggctgatttccaacatcctgcaggggctgtggggccagagcccacagacagaggcctgcagtggaggtccagcccagctgccgatgcccaggaagaaaacctctatgctgccgtgaagcacacacagcctgaggatggggtggagatggacactcggagcccacacgatgaagacccccaggcagtgacgtatgccgaggtgaaacactccagacctaggagagaaatggcctctcctccttccccactgtctggggaattcctggacacaaaggacagacaggcggaagaggacaggcagatggacactgaggctgctgcatctgaagccccccaggatgtgacctacgcccagctgcacagcttgaccctcagacgggaggcaactgagcctcctccatcccaggaagggccctctccagctgtgcccagcatctacgccactctggccatccac

SEQ ID NO: 989 - protein, 519 aa, synthetic construct
  REGION 1..519: note = HLA-B07 inhibitory receptor
MDMRVPAQLLGLLLLWLRGARCDVQLQESGPDLVKPSQSLSLTCTVTGYSITSGYSWHWIRQFPRNKLEWMGYIHFSGSTHYHPSLKSRISITRDTSKNQFFLQLNSVTTEDTATYYCARGGVVSHYAMDCWGQGTSVTVSSGGGGSGGGGSGGGGSGGDIQMTQSPASLSVSVGETVTITCRASENIYSNLAWYQQKQGKSPHLLVYAATYLPDGVPSRFSGSGSGTQYSLKINSLQSEDFGSYYCQHFWVTPYTFGGGTKVEIKYGSQSSKPYLLTHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 990 - DNA, 1557 bp, synthetic construct
  misc_feature 1..1557: note = HLA-B07 inhibitory receptor
  source 1..1557: mol_type = other DNA
atggacatgagggtccccgctcagctcctggggctcctgctactctggctccgaggtgccagatgtgatgtgcagcttcaggagtcaggacctgacctggtgaaaccttctcagtcactttcactcacctgcactgtcactggctactccatcaccagtggttatagctggcactggatccggcagtttccaagaaacaaactggaatggatgggctacatacacttcagtggtagcactcactaccatccatctctcaaaagtcgaatctctatcactcgagacacatccaagaaccagttcttcctgcagttgaattctgtgactactgaggacacagccacatattactgtgcaagagggggggtagtgtcccactatgctatggactgctggggtcagggaacctcagtcaccgtgtcctcaggaggtggcggctctggaggcggaggtagcggaggtggaggctctggtggcgacatccagatgactcagtctccagcctccctatctgtatctgtgggagaaactgtcaccatcacatgtcgagcaagtgagaatatttacagtaatttagcatggtatcagcagaaacagggaaaatctcctcacctcctggtctatgctgcaacatacttaccagatggtgtgccatcaaggttcagtggcagtggatcaggcacacaatattccctcaagatcaacagcctgcagtctgaagattttgggagttattactgtcaacatttttgggttactccgtacacgttcggaggggggaccaaggtggaaataaaatacggctcacagagctccaaaccctacctgctgactcaccccagtgaccccctggagctcgtggtctcaggaccgtctgggggccccagctccccgacaacaggccccacctccacatctggccctgaggaccagcccctcacccccaccgggtcggatccccagagtggtctgggaaggcacctgggggttgtgatcggcatcttggtggccgtcatcctactgctcctcctcctcctcctcctcttcctcatcctccgacatcgacgtcagggcaaacactggacatcgacccagagaaaggctgatttccaacatcctgcaggggctgtggggccagagcccacagacagaggcctgcagtggaggtccagcccagctgccgatgcccaggaagaaaacctctatgctgccgtgaagcacacacagcctgaggatggggtggagatggacactcggagcccacacgatgaagacccccaggcagtgacgtatgccgaggtgaaacactccagacctaggagagaaatggcctctcctccttccccactgtctggggaattcctggacacaaaggacagacaggcggaagaggacaggcagatggacactgaggctgctgcatctgaagccccccaggatgtgacctacgcccagctgcacagcttgaccctcagacgggaggcaactgagcctcctccatcccaggaagggccctctccagctgtgcccagcatctacgccactctggccatccac

SEQ ID NO: 991 - protein, 530 aa, synthetic construct
  REGION 1..530: note = HLA-E CAR
MEFGLSWLFLVAILKGVQCQVQLRESGPSLVKPSQTLSLTCTVSGFSLTSYGVHWVRQPPGKGLEWLGVIWTGGTTNYNTALMSRLSITKDNSKSQVFLKMNSLQTDDTAIYYCARDGDSYNREAWFAYWGQGTLVTVSAGGGGSGGGGSGGGGSGGQIVLTQSPAVISASPGEKVILTCSASSSVSYMHWFQQKPGTSPKLWIYGTSNLASGVPARFSGGGSGTSYSLTISRMEAEDAATYYCQQRSRYPFTFGSGTKLEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 992 - protein, 263 aa, synthetic construct
  REGION 1..263: note = HLA-E scFv
MEFGLSWLFLVAILKGVQCQVQLRESGPSLVKPSQTLSLTCTVSGFSLTSYGVHWVRQPPGKGLEWLGVIWTGGTTNYNTALMSRLSITKDNSKSQVFLKMNSLQTDDTAIYYCARDGDSYNREAWFAYWGQGTLVTVSAGGGGSGGGGSGGGGSGGQIVLTQSPAVISASPGEKVILTCSASSSVSYMHWFQQKPGTSPKLWIYGTSNLASGVPARFSGGGSGTSYSLTISRMEAEDAATYYCQQRSRYPFTFGSGTKLEIK

SEQ ID NO: 993 - protein, 244 aa, synthetic construct
  REGION 1..244: note = BB7.1 scFv
DVQLQESGPDLVKPSQSLSLTCTVTGYSITSGYSWHWIRQFPRNKLEWMGYIHFSGSTHYHPSLKSRISITRDTSKNQFFLQLNSVTTEDTATYYCARGGVVSHYAMDCWGQGTSVTVSSGGGGSGGGGSGGGGSGGDIQMTQSPASLSVSVGETVTITCRASENIYSNLAWYQQKQGKSPHLLVYAATYLPDGVPSRFSGSGSGTQYSLKINSLQSEDFGSYYCQHFWVTPYTFGGGTKVEIK

SEQ ID NO: 994 - protein, 241 aa, synthetic construct
  REGION 1..241: note = GAP A3
EVKLEESGGGLVQPGGSMKLSCVASGFTFSNYWMNWVRQSPEKGLEWVAEIRLKSTNYATHYAESVKGRFTISRDDSKSSVYLQMNNLRAEDTGIYYCTTLITPDYWGQGTTLTVSSGGGGSGGGGSGGGGSGGDIVMTQSHKFMSTSVGDRVSITCKASQDVSTTVAWYQQKPGQSPKLLIYSASYRYTGVPDRFTGSGSGTDFTFTISSVQAEDLAVYYCQQHYSTPPTFGGGTKLEIK

SEQ ID NO: 995 - protein, 11 aa, synthetic construct
  REGION 1..11: note = GAP A3 - CDRL1
KASQDVSTTVA

SEQ ID NO: 996 - protein, 7 aa, synthetic construct
  REGION 1..7: note = GAP A3 - CDRL2
SASYRYT

SEQ ID NO: 997 - protein, 9 aa, synthetic construct
  REGION 1..9: note = GAP A3 - CDRL3
QQHYSTPPT

SEQ ID NO: 998 - protein, 5 aa, synthetic construct
  REGION 1..5: note = GAP A3 - CDRH1
NYWMN

SEQ ID NO: 999 - protein, 19 aa, synthetic construct
  REGION 1..19: note = GAP A3 - CDRH2
EIRLKSTNYATHYAESVKG

SEQ ID NO: 1000 - protein, 6 aa, synthetic construct
  REGION 1..6: note = GAP A3 - CDRH3
LITPDY

SEQ ID NO: 1001 - protein, 107 aa, synthetic construct
  REGION 1..107: note = HLA-A*03 - VL
DIVMTQSHKFMSTSVGDRVSITCKASQDVSTTVAWYQQKPGQSPKLLIYSASYRYTGVPDRFTGSGSGTDFTFTISSVQAEDLAVYYCQQHYSTPPTFGGGTKLEIK

SEQ ID NO: 1002 - protein, 117 aa, synthetic construct
  REGION 1..117: note = HLA-A*03 - VH
EVKLEESGGGLVQPGGSMKLSCVASGFTFSNYWMNWVRQSPEKGLEWVAEIRLKSTNYATHYAESVKGRFTISRDDSKSSVYLQMNNLRAEDTGIYYCTTLITPDYWGQGTTLTVSS

SEQ ID NO: 1003 - protein, 500 aa, synthetic construct
  REGION 1..500: note = HLA-A*02 LIR1 blocker
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPRTSGGGTKLEIKGGGGSGGGGSGGGGSGGQVQLQQSGPELVKPGASVRISCKASGYTFTSYHIHWVKQRPGQGLEWIGWIYPGNVNTEYNEKFKGKATLTADKSSSTAYMHLSSLTSEDSAVYFCAREEITYAMDYWGQGTSVTVSSYGSQSSKPYLLTHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 1004 - protein, 494 aa, synthetic construct
  REGION 1..494: note = HLA-A*03 inhibitory receptor
EVKLEESGGGLVQPGGSMKLSCVASGFTFSNYWMNWVRQSPEKGLEWVAEIRLKSTNYATHYAESVKGRFTISRDDSKSSVYLQMNNLRAEDTGIYYCTTLITPDYWGQGTTLTVSSGGGGSGGGGSGGGGSGGDIVMTQSHKFMSTSVGDRVSITCKASQDVSTTVAWYQQKPGQSPKLLIYSASYRYTGVPDRFTGSGSGTDFTFTISSVQAEDLAVYYCQQHYSTPPTFGGGTKLEIKYGSQSSKPYLLTHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 1005 - DNA, 1548 bp, synthetic construct
  misc_feature 1..1548: note = HLA-A*03 inhibitory receptor
  source 1..1548: mol_type = other DNA
atggacatgagggtccccgctcagctcctggggctcctgctactctggctccgaggtgccagatgtgaagtgaagcttgaggagtctggaggaggcttggtgcaacctggaggatctatgaaactctcctgtgttgcctctggattcactttcagtaactactggatgaactgggtccgccagtctccagagaaggggcttgagtgggttgctgaaattagattgaaatctactaattatgcaacacattatgcggagtctgtgaaagggaggttcaccatctcaagagatgattccaaaagtagtgtctacctgcaaatgaacaacttaagagctgaagacactggcatttattactgtactactctgattacccctgactactggggccaaggcaccactctcacagtctcctcaggaggtggcggctctggaggcggaggtagcggaggtggaggctctggtggcgacattgtgatgacccagtctcacaaattcatgtccacatcagtaggagacagggtcagcatcacctgcaaggccagtcaggatgtgagtactactgtggcctggtatcagcagaaaccagggcaatctcctaaactactgatttattcggcatcctaccggtacactggagtccctgatcgcttcactggcagtggatctgggacggatttcactttcaccatcagcagtgtgcaggctgaagacctggcagtttattactgtcagcaacattatagtactcctcccacgttcggaggggggaccaagctggaaataaaatacggctcacagagctccaaaccctacctgctgactcaccccagtgaccccctggagctcgtggtctcaggaccgtctgggggccccagctccccgacaacaggccccacctccacatctggccctgaggaccagcccctcacccccaccgggtcggatccccagagtggtctgggaaggcacctgggggttgtgatcggcatcttggtggccgtcatcctactgctcctcctcctcctcctcctcttcctcatcctccgacatcgacgtcagggcaaacactggacatcgacccagagaaaggctgatttccaacatcctgcaggggctgtggggccagagcccacagacagaggcctgcagtggaggtccagcccagctgccgatgcccaggaagaaaacctctatgctgccgtgaagcacacacagcctgaggatggggtggagatggacactcggagcccacacgatgaagacccccaggcagtgacgtatgccgaggtgaaacactccagacctaggagagaaatggcctctcctccttccccactgtctggggaattcctggacacaaaggacagacaggcggaagaggacaggcagatggacactgaggctgctgcatctgaagccccccaggatgtgacctacgcccagctgcacagcttgaccctcagacgggaggcaactgagcctcctccatcccaggaagggccctctccagctgtgcccagcatctacgccactctggccatccac

SEQ ID NO: 1006 - protein, 496 aa, synthetic construct
  REGION 1..496: note = HLA-A*11 inhibitory receptor
EVQLVESGGGLVQPGGSLRLSCAASGFTFSSYWMHWVRQAPGKGLVWVSRINSDGSSTSYADSVKGRFTISRDNAKNTLYLQMNSLRAEDTAVYYCCLGVLLYNWFDPWGQGTLVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKYGSQSSKPYLLTHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 1007 - DNA, 1554 bp, synthetic construct; identical to SEQ ID NO: 988
  misc_feature 1..1554: note = HLA-A*11 inhibitory receptor
  source 1..1554: mol_type = other DNA

SEQ ID NO: 1008 - protein, 497 aa, synthetic construct
  REGION 1..497: note = HLA-B*07 inhibitory receptor
DVQLQESGPDLVKPSQSLSLTCTVTGYSITSGYSWHWIRQFPRNKLEWMGYIHFSGSTHYHPSLKSRISITRDTSKNQFFLQLNSVTTEDTATYYCARGGVVSHYAMDCWGQGTSVTVSSGGGGSGGGGSGGGGSGGDIQMTQSPASLSVSVGETVTITCRASENIYSNLAWYQQKQGKSPHLLVYAATYLPDGVPSRFSGSGSGTQYSLKINSLQSEDFGSYYCQHFWVTPYTFGGGTKVEIKYGSQSSKPYLLTHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 1009 - DNA, 732 bp, synthetic construct
  misc_feature 1..732: note = HLA-B*07 inhibitory receptor
  source 1..732: mol_type = other DNA
gatgtgcagcttcaggagtcaggacctgacctggtgaaaccttctcagtcactttcactcacctgcactgtcactggctactccatcaccagtggttatagctggcactggatccggcagtttccaagaaacaaactggaatggatgggctacatacacttcagtggtagcactcactaccatccatctctcaaaagtcgaatctctatcactcgagacacatccaagaaccagttcttcctgcagttgaattctgtgactactgaggacacagccacatattactgtgcaagagggggggtagtgtcccactatgctatggactgctggggtcagggaacctcagtcaccgtgtcctcaggaggtggcggctctggaggcggaggtagcggaggtggaggctctggtggcgacatccagatgactcagtctccagcctccctatctgtatctgtgggagaaactgtcaccatcacatgtcgagcaagtgagaatatttacagtaatttagcatggtatcagcagaaacagggaaaatctcctcacctcctggtctatgctgcaacatacttaccagatggtgtgccatcaaggttcagtggcagtggatcaggcacacaatattccctcaagatcaacagcctgcagtctgaagattttgggagttattactgtcaacatttttgggttactccgtacacgttcggaggggggaccaaggtggaaataaaa

SEQ ID NO: 1010 - protein, 13 aa, synthetic construct
  REGION 1..13: note = HLA-A*03 CDR H3
GGDLSHYYYYMDV

SEQ ID NO: 1011 - DNA, 3222 bp, synthetic construct
  misc_feature 1..3222: note = murine inhibitor receptor -T2A-activator receptor
  source 1..3222: mol_type = other DNA
atggacatgagggtccccgctcagctcctggggctcctgctactctggctccgaggtgccagatgtgatgttttgatgacccaaactccactctccctgcctgtcagtcttggagatcaagcctccatctcttgcagatctagtcagagcattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagtagagtggaggctgaggatctgggagtttattactgctttcaaggttcacatgttcctcggacgtccggtggaggcaccaagctggaaatcaaaggcggaggtggaagcggagggggaggatctggcggcggaggaagcggaggccaggtccagctgcagcagtctggacctgagctggtgaagcctggggcttcagtgaggatatcctgcaaggcttctggctacaccttcacaagttaccatatacattgggtgaagcagaggcctggacagggacttgagtggattggatggatttatcctggaaatgttaatactgagtacaatgagaagttcaagggcaaggccacactgactgcagacaaatcgtccagcacagcctacatgcacctcagcagcctgacctctgaggactctgcggtctatttctgtgccagagaggagattacctatgctatggactactggggtcaaggaacctcagtcaccgtgtcctcatacggctcacagagctccaaaccctacctgctgactcaccccagtgaccccctggagctcgtggtctcaggaccgtctgggggccccagctccccgacaacaggccccacctccacatctggccctgaggaccagcccctcacccccaccgggtcggatccccagagtggtctgggaaggcacctgggggttgtgatcggcatcttggtggccgtcatcctactgctcctcctcctcctcctcctcttcctcatcctccgacatcgacgtcagggcaaacactggacatcgacccagagaaaggctgatttccaacatcctgcaggggctgtggggccagagcccacagacagaggcctgcagtggaggtccagcccagctgccgatgcccaggaagaaaacctctatgctgccgtgaagcacacacagcctgaggatggggtggagatggacactcggagcccacacgatgaagacccccaggcagtgacgtatgccgaggtgaaacactccagacctaggagagaaatggcctctcctccttccccactgtctggggaattcctggacacaaaggacagacaggcggaagaggacaggcagatggacactgaggctgctgcatctgaagccccccaggatgtgacctacgcccagctgcacagcttgaccctcagacgggaggcaactgagcctcctccatcccaggaagggccctctccagctgtgcccagcatctacgccactctggccatccacggatccggagagggcagaggcagcctgctgacatgtggcgacgtggaagagaaccctggccccatggagtttgggctgagctggctttttcttgtggctattttaaaaggtgtccagtgtcaggtgcagctgagggagagcggccccagcctggtgaagcccagccagaccctgagcctgacctgcaccgtgagcggcttcagcctgaccagctacggcgtgcactgggtgaggcagccccccggcaagggcctggagtggctgggcgtgatctggaccggcggcaccaccaactacaacaccgccctgatgagcaggctgagcatcaccaaggacaacagcaagagccaggtgttcctgaagatgaacagcctgcagaccgacgacaccgccatctactactgcgccagggacggcgacagctacaacagggaggcctggttcgcctactggggccagggcaccctggtgaccgtgagcgccggcggaggtggaagcggagggggaggatctggcggcggaggaagcggaggccagatcgtgctgacccagagccccgccgtgatcagcgccagccccggcgagaaggtgatcctgacctgcagcgccagcagcagcgtgagctacatgcactggttccagcagaagcccggcaccagccccaagctgtggatctacggcaccagcaacctggccagcggcgtgcccgccaggttcagcggcggcggcagcggcaccagctacagcctgaccatcagcaggatggaggccgaggacgccgccacctactactgccagcagaggagcaggtaccccttcaccttcggcagcggcaccaagctggagatcaagaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttctgggtgctggtcgttgtgggcggcgtgctggcctgctacagcctgctggtgacagtggccttcatcatcttttgggtgaggagcaagcggagcagactgctgcacagcgactacatgaacatgaccccccggaggcctggccccacccggaagcactaccagccctacgcccctcccagggatttcgccgcctaccggagcaaacggggcagaaagaaactcctgtatatattcaaacaaccatttatgaggccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagcgtagaggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggactcagtacagccaccaaggacacctacgacgcccttcacatgcaggccctgccccctcgctag

SEQ ID NO: 1012 - protein, 1073 aa, synthetic construct
  REGION 1..1073: note = murine inhibitor receptor-T2A-activator receptor
MDMRVPAQLLGLLLLWLRGARCDVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPRTSGGGTKLEIKGGGGSGGGGSGGGGSGGQVQLQQSGPELVKPGASVRISCKASGYTFTSYHIHWVKQRPGQGLEWIGWIYPGNVNTEYNEKFKGKATLTADKSSSTAYMHLSSLTSEDSAVYFCAREEITYAMDYWGQGTSVTVSSYGSQSSKPYLLTHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIHGSGEGRGSLLTCGDVEENPGPMEFGLSWLFLVAILKGVQCQVQLRESGPSLVKPSQTLSLTCTVSGFSLTSYGVHWVRQPPGKGLEWLGVIWTGGTTNYNTALMSRLSITKDNSKSQVFLKMNSLQTDDTAIYYCARDGDSYNREAWFAYWGQGTLVTVSAGGGGSGGGGSGGGGSGGQIVLTQSPAVISASPGEKVILTCSASSSVSYMHWFQQKPGTSPKLWIYGTSNLASGVPARFSGGGSGTSYSLTISRMEAEDAATYYCQQRSRYPFTFGSGTKLEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 1013 - DNA, 3222 bp, synthetic construct
  misc_feature 1..3222: note = humanized inhibitor receptor-T2A-activator receptor
  source 1..3222: mol_type = other DNA
atggacatgagggtccccgctcagctcctggggctcctgctactctggctccgaggtgccagatgtcaggtgcagctggtgcagtctggggctgaggtgaagaagcctgggtcctcagtgaaggtttcctgcaaggcttctggatacaccttcactagctatcatatacattgggtgcgccaggcccccggacaagggcttgagtggatcggatggatctaccctggcaatgttaacacagaatataatgagaagttcaagggcaaagccaccattaccgcggacgaatccacgaacacagcctacatggagctgagcagcctgagatctgaagacacggctgtgtattactgtgcgagggaggaaattacctacgctatggactactggggccagggaaccctggtcaccgtgtcctcaggcggaggtggaagcggagggggaggatctggcggcggaggaagcggaggcgacattcaaatgacccagagcccatccaccctgagcgcatctgtaggtgaccgggtcaccatcacttgtagatccagtcagagtattgtacacagtaatgggaacacctatttggaatggtatcagcagaaaccaggtaaagccccaaaattgctcatctacaaagtctctaacagatttagtggtgtaccagccaggttcagcggttccggaagtggtactgaattcaccctcacgatctcctctctccagccagatgatttcgccacttattactgttttcaaggttcacatgtgccgcgcacattcggtcagggtactaaagtagaagtcaaatacggctcacagagctccaaaccctacctgctgactcaccccagtgaccccctggagctcgtggtctcaggaccgtctgggggccccagctccccgacaacaggccccacctccacatctggccctgaggaccagcccctcacccccaccgggtcggatccccagagtggtctgggaaggcacctgggggttgtgatcggcatcttggtggccgtcatcctactgctcctcctcctcctcctcctcttcctcatcctccgacatcgacgtcagggcaaacactggacatcgacccagagaaaggctgatttccaacatcctgcaggggctgtggggccagagcccacagacagaggcctgcagtggaggtccagcccagctgccgatgcccaggaagaaaacctctatgctgccgtgaagcacacacagcctgaggatggggtggagatggacactcggagcccacacgatgaagacccccaggcagtgacgtatgccgaggtgaaacactccagacctaggagagaaatggcctctcctccttccccactgtctggggaattcctggacacaaaggacagacaggcggaagaggacaggcagatggacactgaggctgctgcatctgaagccccccaggatgtgacctacgcccagctgcacagcttgaccctcagacgggaggcaactgagcctcctccatcccaggaagggccctctccagctgtgcccagcatctacgccactctggccatccacggatccggagagggcagaggcagcctgctgacatgtggcgacgtggaagagaaccctggccccatggagtttgggctgagctggctttttcttgtggctattttaaaaggtgtccagtgtcaggtgcagctgagggagagcggccccagcctggtgaagcccagccagaccctgagcctgacctgcaccgtgagcggcttcagcctgaccagctacggcgtgcactgggtgaggcagccccccggcaagggcctggagtggctgggcgtgatctggaccggcggcaccaccaactacaacaccgccctgatgagcaggctgagcatcaccaaggacaacagcaagagccaggtgttcctgaagatgaacagcctgcagaccgacgacaccgccatctactactgcgccagggacggcgacagctacaacagggaggcctggttcgcctactggggccagggcaccctggtgaccgtgagcgccggcggaggtggaagcggagggggaggatctggcggcggaggaagcggaggccagatcgtgctgacccagagccccgccgtgatcagcgccagccccggcgagaaggtgatcctgacctgcagcgccagcagcagcgtgagctacatgcactggttccagcagaagcccggcaccagccccaagctgtggatctacggcaccagcaacctggccagcggcgtgcccgccaggttcagcggcggcggcagcggcaccagctacagcctgaccatcagcaggatggaggccgaggacgccgccacctactactgccagcagaggagcaggtaccccttcaccttcggcagcggcaccaagctggagatcaagaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttctgggtgctggtcgttgtgggcggcgtgctggcctgctacagcctgctggtgacagtggccttcatcatcttttgggtgaggagcaagcggagcagactgctgcacagcgactacatgaacatgaccccccggaggcctggccccacccggaagcactaccagccctacgcccctcccagggatttcgccgcctaccggagcaaacggggcagaaagaaactcctgtatatattcaaacaaccatttatgaggccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagcgtagaggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggactcagtacagccaccaaggacacctacgacgcccttcacatgcaggccctgccccctcgctag

SEQ ID NO: 1014 - protein, 1866 aa, synthetic construct
  REGION 1..1866: note = humanized inhibitor receptor-T2A-activator receptor
MDMRVPAQLLGLLLLWLRGARCQVQLVQSGAEVKKPGSSVKVSCKASGYTFTSYHIHWVRQAPGQGLEWIGWIYPGNVNTEYNEKFKGKATITADESTNTAYMELSSLRSEDTAVYYCAREEITYAMDYWGQGTLVTVSSGGGGSGGGGSGGGGSGGDIQMTQSPSTLSASVGDRVTITCRSSQSIVHSNGNTYLEWYQQKPGKAPKLLIYKVSNRFSGVPARFSGSGSGTEFTLTISSLQPDDFATYYCFQGSHVPRTFGQGTKVEVKYGSQSSKPYLLTHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIHGSGEGRGSLLTCGDVEENPGPMEFGLSWLFLVAILKGVQCQVQLRESGPSLVKPSQTLSLTCTVSGFSLTSYGVHWVRQPPGKGLEWLGVIWTGGTTNYNTALMSRLSITKDNSKSQVFLKMNSLQTDDTAIYYCARDGDSYNREAWFAYWGQGTLVTVSAGGGGSGGGGSGGGGSGGQIVLTQSPAVISASPGEKVILTCSASSSVSYMHWFQQKPGTSPKLWIYGTSNLASGVPARFSGGGSGTSYSLTISRMEAEDAATYYCQQRSRYMDMRVPAQLLGLLLLWLRGARCQVQLVQSGAEVKKPGSSVKVSCKASGYTFTSYHIHWVRQAPGQGLEWIGWIYPGNVNTEYNEKFKGKATITADESTNTAYMELSSLRSEDTAVYYCAREEITYAMDYWGQGTLVTVSSGGGGSGGGGSGGGGSGGDIQMTQSPSTLSASVGDRVTITCRSSQSIVHSNGNTYLEWYQQKPGKAPKLLIYKVSNRFSGVPARFSGSGSGTEFTLTISSLQPDDFATYYCFQGSHVPRTFGQGTKVEVKYGSQSSKPYLLTHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIHGSGEGRGSLLTCGDVEENPGPMEFGLSWLFLVAILKGVQCQVQLRESGPSLVKPSQTLSLTCTVSGFSLTSYGVHWVRQPPGKGLEWLGVIWTGGTTNYNTALMSRLSITKDNSKSQVFLKMNSLQTDDTAIYYCARDGDSYNREAWFAYWGQGTLVTVSAGGGGSGGGGSGGGGSGGQIVLTQSPAVISASPGEKVILTCSASSSVSYMHWFQQKPGTSPKLWIYGTSNLASGVPARFSGGGSGTSYSLTISRMEAEDAATYYCQQRSRYPFTFGSGTKLEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 1015 - DNA, 51 bp, synthetic construct
  misc_feature 1..51: note = glycine serine linker
  source 1..51: mol_type = other DNA
ggcggaggtggaagcggagggggaggatctggcggcggaggaagcggaggc

SEQ ID NO: 1016 - DNA, 363 bp, synthetic construct; identical to SEQ ID NO: 980
  misc_feature 1..363: note = anti-HLA-E VH
  source 1..363: mol_type = other DNA

SEQ ID NO: 1017 - protein, 5 aa, synthetic construct
  REGION 1..5: note = anti-HLA-E CDRH1
SYGVH

SEQ ID NO: 1018 - protein, 16 aa, synthetic construct
  REGION 1..16: note = anti-HLA-E CDRH2
VIWTGGTTNYNTALMS

SEQ ID NO: 1019 - DNA, 588 bp, synthetic construct
  misc_feature 1..588: note = CD28-41BB-CD3epsilon intracellular domain
  source 1..588: mol_type = other DNA
aggagcaagcggagcagactgctgcacagcgactacatgaacatgaccccccggaggcctggccccacccggaagcactaccagccctacgcccctcccagggatttcgccgcctaccggagcaaacggggcagaaagaaactcctgtatatattcaaacaaccatttatgaggccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagcgtagaggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggactcagtacagccaccaaggacacctacgacgcccttcacatgcaggccctgccccctcgctag

SEQ ID NO: 1020 - DNA, 339 bp, synthetic construct
  misc_feature 1..339: note = CD3epsilon activation domain
  source 1..339: mol_type = other DNA
agagtgaagttcagcaggagcgcagacgcccccgcgtacaagcagggccagaaccagctctataacgagctcaatctaggacgaagagaggagtacgatgttttggacaagcgtagaggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggactcagtacagccaccaaggacacctacgacgcccttcacatgcaggccctgccccctcgctag

SEQ ID NO: 1021 - protein, 522 aa, synthetic construct
  REGION 1..522: note = murine HLA-A*02 inhibitor receptor
MDMRVPAQLLGLLLLWLRGARCDVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPRTSGGGTKLEIKGGGGSGGGGSGGGGSGGQVQLQQSGPELVKPGASVRISCKASGYTFTSYHIHWVKQRPGQGLEWIGWIYPGNVNTEYNEKFKGKATLTADKSSSTAYMHLSSLTSEDSAVYFCAREEITYAMDYWGQGTSVTVSSYGSQSSKPYLLTHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 1022 - DNA, 1569 bp, synthetic construct
  misc_feature 1..1569: note = murine HLA-A*02 inhibitor receptor
  source 1..1569: mol_type = other DNA
atggacatgagggtccccgctcagctcctggggctcctgctactctggctccgaggtgccagatgtgatgttttgatgacccaaactccactctccctgcctgtcagtcttggagatcaagcctccatctcttgcagatctagtcagagcattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagtagagtggaggctgaggatctgggagtttattactgctttcaaggttcacatgttcctcggacgtccggtggaggcaccaagctggaaatcaaaggcggaggtggaagcggagggggaggatctggcggcggaggaagcggaggccaggtccagctgcagcagtctggacctgagctggtgaagcctggggcttcagtgaggatatcctgcaaggcttctggctacaccttcacaagttaccatatacattgggtgaagcagaggcctggacagggacttgagtggattggatggatttatcctggaaatgttaatactgagtacaatgagaagttcaagggcaaggccacactgactgcagacaaatcgtccagcacagcctacatgcacctcagcagcctgacctctgaggactctgcggtctatttctgtgccagagaggagattacctatgctatggactactggggtcaaggaacctcagtcaccgtgtcctcatacggctcacagagctccaaaccctacctgctgactcaccccagtgaccccctggagctcgtggtctcaggaccgtctgggggccccagctccccgacaacaggccccacctccacatctggccctgaggaccagcccctcacccccaccgggtcggatccccagagtggtctgggaaggcacctgggggttgtgatcggcatcttggtggccgtcatcctactgctcctcctcctcctcctcctcttcctcatcctccgacatcgacgtcagggcaaacactggacatcgacccagagaaaggctgatttccaacatcctgcaggggctgtggggccagagcccacagacagaggcctgcagtggaggtccagcccagctgccgatgcccaggaagaaaacctctatgctgccgtgaagcacacacagcctgaggatggggtggagatggacactcggagcccacacgatgaagacccccaggcagtgacgtatgccgaggtgaaacactccagacctaggagagaaatggcctctcctccttccccactgtctggggaattcctggacacaaaggacagacaggcggaagaggacaggcagatggacactgaggctgctgcatctgaagccccccaggatgtgacctacgcccagctgcacagcttgaccctcagacgggaggcaactgagcctcctccatcccaggaagggccctctccagctgtgcccagcatctacgccactctggccatccactag

SEQ ID NO: 1023 - protein, 7 aa, synthetic construct
  REGION 1..7: note = anti-HLA-A*02 CDRL2
KVSNRFS

SEQ ID NO: 1024 - protein, 5 aa, synthetic construct
  REGION 1..5: note = anti-HLA-A*02 CDRH1
SYHIH

SEQ ID NO: 1025 - protein, 17 aa, synthetic construct
  REGION 1..17: note = anti-HLA-A*02 CDRH2
WIYPGNVNTEYNEKFKG

SEQ ID NO: 1026 - protein, 112 aa, synthetic construct
  REGION 1..112: note = anti-HLA-A*02 VL
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPRTSGGGTKLEIK

SEQ ID NO: 1027 - DNA, 336 bp, synthetic construct
  misc_feature 1..336: note = anti-HLA-A*02 VL
  source 1..336: mol_type = other DNA
gatgttttgatgacccaaactccactctccctgcctgtcagtcttggagatcaagcctccatctcttgcagatctagtcagagcattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagtagagtggaggctgaggatctgggagtttattactgctttcaaggttcacatgttcctcggacgtccggtggaggcaccaagctggaaatcaaa

SEQ ID NO: 1028 - protein, 118 aa, synthetic construct
  REGION 1..118: note = anti-HLA-A*02 VH
QVQLQQSGPELVKPGASVRISCKASGYTFTSYHIHWVKQRPGQGLEWIGWIYPGNVNTEYNEKFKGKATLTADKSSSTAYMHLSSLTSEDSAVYFCAREEITYAMDYWGQGTSVTVSS

SEQ ID NO: 1029 - DNA, 354 bp, synthetic construct
  misc_feature 1..354: note = anti-HLA-A*02 VH
  source 1..354: mol_type = other DNA
caggtccagctgcagcagtctggacctgagctggtgaagcctggggcttcagtgaggatatcctgcaaggcttctggctacaccttcacaagttaccatatacattgggtgaagcagaggcctggacagggacttgagtggattggatggatttatcctggaaatgttaatactgagtacaatgagaagttcaagggcaaggccacactgactgcagacaaatcgtccagcacagcctacatgcacctcagcagcctgacctctgaggactctgcggtctatttctgtgccagagaggagattacctatgctatggactactggggtcaaggaacctcagtcaccgtgtcctca

SEQ ID NO: 1030 - DNA, 189 bp, synthetic construct
  misc_feature 1..189: note = LIR1 hinge
  source 1..189: mol_type = other DNA
tacggctcacagagctccaaaccctacctgctgactcaccctagtgatcctctggagctcgtggtctcaggaccgtctggaggcccaagctctccgacaacaggccccacctccacatctggccctgaggaccagcccctcacacccaccgggtcggatcctcagagtggtctgggaagacacctggga

SEQ ID NO: 1031 - DNA, 69 bp, synthetic construct
  misc_feature 1..69: note = LIR1 transmembrane domain
  source 1..69: mol_type = other DNA
gttgtgatcggcatcttggtggccgtcatcctactgctcctcctcctgctcctgctcttcctcatcctc

SEQ ID NO: 1032 - DNA, 501 bp, synthetic construct
  misc_feature 1..501: note = LIR1 intracellular domain
  source 1..501: mol_type = other DNA
cgacatcgacgtcagggcaaacactggacatcgacccagagaaaggctgatttccaacatcctgcaggggctgtggggccagagcccacagacagaggcctgcagtggaggtccagcccagctgccgatgcccaggaagaaaacctctatgctgccgtgaagcacacacagcctgaggatggggtggagatggatactcggagcccacacgatgaagatccacaggcagtgacgtatgccgaggtgaaacactccagacctagaagggaaatggcctctcctccttccccactgtctggagagttcctggacacaaaggacagacaggcggaagaggacaggcagatggacactgaggctgctgcatctgaagctcctcaggatgtgacctacgcccagctgcacagcttgaccctcagacgggaggcaactgagcctcctccatcccaggaagggccctctccagctgtgcccagcatctacgccactctggccatccac

SEQ ID NO: 1033 - protein, 522 aa, synthetic construct
  REGION 1..522: note = humanized anti-HLA-A*02 inhibitor receptor
MDMRVPAQLLGLLLLWLRGARCQVQLVQSGAEVKKPGSSVKVSCKASGYTFTSYHIHWVRQAPGQGLEWIGWIYPGNVNTEYNEKFKGKATITADESTNTAYMELSSLRSEDTAVYYCAREEITYAMDYWGQGTLVTVSSGGGGSGGGGSGGGGSGGDIQMTQSPSTLSASVGDRVTITCRSSQSIVHSNGNTYLEWYQQKPGKAPKLLIYKVSNRFSGVPARFSGSGSGTEFTLTISSLQPDDFATYYCFQGSHVPRTFGQGTKVEVKYGSQSSKPYLLTHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 1034 - DNA, 1566 bp, synthetic construct
  misc_feature 1..1566: note = humanized anti-HAL-A*02 inhibitor receptor
  source 1..1566: mol_type = other DNA
atggatatgagagtgcctgcccagctgctcggactgctccttctgtggttgagaggagctcggtgccaggtgcagctggtgcagtctggggctgaggtgaagaagcctgggtcctcagtgaaggtttcctgcaaggcttctggatacaccttcactagctatcatatacattgggtgcgccaggcccccggacaagggcttgagtggatcggatggatctaccctggcaatgttaacacagaatataatgagaagttcaagggcaaagccaccattaccgcggacgaatccacgaacacagcctacatggagctgagcagcctgagatctgaagacacggctgtgtattactgtgcgagggaggaaattacctacgctatggactactggggccagggaaccctggtcaccgtgtcctcaggaggtggcggctctggaggcggaggtagcggaggtggaggctctggtggcgacattcaaatgacccagagcccatccaccctgagcgcatctgtaggtgaccgggtcaccatcacttgtagatccagtcagagtattgtacacagtaatgggaacacctatttggaatggtatcagcagaaaccaggtaaagccccaaaattgctcatctacaaagtctctaacagatttagtggtgtaccagccaggttcagcggttccggaagtggtactgaattcaccctcacgatctcctctctccagccagatgatttcgccacttattactgttttcaaggttcacatgtgccgcgcacattcggtcagggtactaaagtagaagtcaaatacggctcacagagctccaaaccctacctgctgactcaccctagtgatcctctggagctcgtggtctcaggaccgtctggaggcccaagctctccgacaacaggccccacctccacatctggccctgaggaccagcccctcacacccaccgggtcggatcctcagagtggtctgggaagacacctgggagttgtgatcggcatcttggtggccgtcatcctactgctcctcctcctgctcctgctcttcctcatcctccgacatcgacgtcagggcaaacactggacatcgacccagagaaaggctgatttccaacatcctgcaggggctgtggggccagagcccacagacagaggcctgcagtggaggtccagcccagctgccgatgcccaggaagaaaacctctatgctgccgtgaagcacacacagcctgaggatggggtggagatggatactcggagcccacacgatgaagatccacaggcagtgacgtatgccgaggtgaaacactccagacctagaagggaaatggcctctcctccttccccactgtctggagagttcctggacacaaaggacagacaggcggaagaggacaggcagatggacactgaggctgctgcatctgaagctcctcaggatgtgacctacgcccagctgcacagcttgaccctcagacgggaggcaactgagcctcctccatcccaggaagggccctctccagctgtgcccagcatctacgccactctggccatccac

SEQ ID NO: 1035 - protein, 112 aa, synthetic construct
  REGION 1..112: note = humanized anti-HLA-A*02 VL
DIQMTQSPSTLSASVGDRVTITCRSSQSIVHSNGNTYLEWYQQKPGKAPKLLIYKVSNRFSGVPARFSGSGSGTEFTLTISSLQPDDFATYYCFQGSHVPRTFGQGTKVEVK

SEQ ID NO: 1036 - DNA, 336 bp, synthetic construct
  misc_feature 1..336: note = humanized anti-HLA-A*02 VL
  source 1..336: mol_type = other DNA
gacattcaaatgacccagagcccatccaccctgagcgcatctgtaggtgaccgggtcaccatcacttgtagatccagtcagagtattgtacacagtaatgggaacacctatttggaatggtatcagcagaaaccaggtaaagccccaaaattgctcatctacaaagtctctaacagatttagtggtgtaccagccaggttcagcggttccggaagtggtactgaattcaccctcacgatctcctctctccagccagatgatttcgccacttattactgttttcaaggttcacatgtgccgcgcacattcggtcagggtactaaagtagaagtcaaa

SEQ ID NO: 1037 - protein, 118 aa, synthetic construct
  REGION 1..118: note = humanized anti-HLA-A*02 VH
QVQLVQSGAEVKKPGSSVKVSCKASGYTFTSYHIHWVRQAPGQGLEWIGWIYPGNVNTEYNEKFKGKATITADESTNTAYMELSSLRSEDTAVYYCAREEITYAMDYWGQGTLVTVSS

SEQ ID NO: 1038 - DNA, 354 bp, synthetic construct
  misc_feature 1..354: note = humanized anti-HLA-A*02 VH
  source 1..354: mol_type = other DNA
caggtgcagctggtgcagtctggggctgaggtgaagaagcctgggtcctcagtgaaggtttcctgcaaggcttctggatacaccttcactagctatcatatacattgggtgcgccaggcccccggacaagggcttgagtggatcggatggatctaccctggcaatgttaacacagaatataatgagaagttcaagggcaaagccaccattaccgcggacgaatccacgaacacagcctacatggagctgagcagcctgagatctgaagacacggctgtgtattactgtgcgagggaggaaattacctacgctatggactactggggccagggaaccctggtcaccgtgtcctca

SEQ ID NO: 1039-1043 - skipped (5 entries)

SEQ ID NO: 1044 - protein, 7 aa, synthetic construct
  REGION 1..7: note = HLA-C*07 CDR-H1
SNSAAWN

SEQ ID NO: 1045 - protein, 18 aa, synthetic construct
  REGION 1..18: note = HLA-C*07 CDR-H2
RTYYRSKWYNDYAVSVKS

SEQ ID NO: 1046 - DNA, 3156 bp, synthetic construct
  misc_feature 1..3156: note = inhibitor receptor-T2A-activator receptor
  source 1..3156: mol_type = other DNA
gatgttttgatgacccaaactccactctccctgcctgtcagtcttggagatcaagcctccatctcttgcagatctagtcagagcattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagtagagtggaggctgaggatctgggagtttattactgctttcaaggttcacatgttcctcggacgtccggtggaggcaccaagctggaaatcaaaggcggaggtggaagcggagggggaggatctggcggcggaggaagcggaggccaggtccagctgcagcagtctggacctgagctggtgaagcctggggcttcagtgaggatatcctgcaaggcttctggctacaccttcacaagttaccatatacattgggtgaagcagaggcctggacagggacttgagtggattggatggatttatcctggaaatgttaatactgagtacaatgagaagttcaagggcaaggccacactgactgcagacaaatcgtccagcacagcctacatgcacctcagcagcctgacctctgaggactctgcggtctatttctgtgccagagaggagattacctatgctatggactactggggtcaaggaacctcagtcaccgtgtcctcatacggctcacagagctccaaaccctacctgctgactcaccccagtgaccccctggagctcgtggtctcaggaccgtctgggggccccagctccccgacaacaggccccacctccacatctggccctgaggaccagcccctcacccccaccgggtcggatccccagagtggtctgggaaggcacctgggggttgtgatcggcatcttggtggccgtcatcctactgctcctcctcctcctcctcctcttcctcatcctccgacatcgacgtcagggcaaacactggacatcgacccagagaaaggctgatttccaacatcctgcaggggctgtggggccagagcccacagacagaggcctgcagtggaggtccagcccagctgccgatgcccaggaagaaaacctctatgctgccgtgaagcacacacagcctgaggatggggtggagatggacactcggagcccacacgatgaagacccccaggcagtgacgtatgccgaggtgaaacactccagacctaggagagaaatggcctctcctccttccccactgtctggggaattcctggacacaaaggacagacaggcggaagaggacaggcagatggacactgaggctgctgcatctgaagccccccaggatgtgacctacgcccagctgcacagcttgaccctcagacgggaggcaactgagcctcctccatcccaggaagggccctctccagctgtgcccagcatctacgccactctggccatccacggatccggagagggcagaggcagcctgctgacatgtggcgacgtggaagagaaccctggccccatggagtttgggctgagctggctttttcttgtggctattttaaaaggtgtccagtgtcaggtgcagctgagggagagcggccccagcctggtgaagcccagccagaccctgagcctgacctgcaccgtgagcggcttcagcctgaccagctacggcgtgcactgggtgaggcagccccccggcaagggcctggagtggctgggcgtgatctggaccggcggcaccaccaactacaacaccgccctgatgagcaggctgagcatcaccaaggacaacagcaagagccaggtgttcctgaagatgaacagcctgcagaccgacgacaccgccatctactactgcgccagggacggcgacagctacaacagggaggcctggttcgcctactggggccagggcaccctggtgaccgtgagcgccggcggaggtggaagcggagggggaggatctggcggcggaggaagcggaggccagatcgtgctgacccagagccccgccgtgatcagcgccagccccggcgagaaggtgatcctgacctgcagcgccagcagcagcgtgagctacatgcactggttccagcagaagcccggcaccagccccaagctgtggatctacggcaccagcaacctggccagcggcgtgcccgccaggttcagcggcggcggcagcggcaccagctacagcctgaccatcagcaggatggaggccgaggacgccgccacctactactgccagcagaggagcaggtaccccttcaccttcggcagcggcaccaagctggagatcaagaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttctgggtgctggtcgttgtgggcggcgtgctggcctgctacagcctgctggtgacagtggccttcatcatcttttgggtgaggagcaagcggagcagactgctgcacagcgactacatgaacatgaccccccggaggcctggccccacccggaagcactaccagccctacgcccctcccagggatttcgccgcctaccggagcaaacggggcagaaagaaactcctgtatatattcaaacaaccatttatgaggccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagcgtagaggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggactcagtacagccaccaaggacacctacgacgcccttcacatgcaggccctgccccctcgctag

SEQ ID NO: 1047 - protein, 1051 aa, synthetic construct
  REGION 1..1051: note = inhibitor receptor-T2A-activator receptor
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPRTSGGGTKLEIKGGGGSGGGGSGGGGSGGQVQLQQSGPELVKPGASVRISCKASGYTFTSYHIHWVKQRPGQGLEWIGWIYPGNVNTEYNEKFKGKATLTADKSSSTAYMHLSSLTSEDSAVYFCAREEITYAMDYWGQGTSVTVSSYGSQSSKPYLLTHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIHGSGEGRGSLLTCGDVEENPGPMEFGLSWLFLVAILKGVQCQVQLRESGPSLVKPSQTLSLTCTVSGFSLTSYGVHWVRQPPGKGLEWLGVIWTGGTTNYNTALMSRLSITKDNSKSQVFLKMNSLQTDDTAIYYCARDGDSYNREAWFAYWGQGTLVTVSAGGGGSGGGGSGGGGSGGQIVLTQSPAVISASPGEKVILTCSASSSVSYMHWFQQKPGTSPKLWIYGTSNLASGVPARFSGGGSGTSYSLTISRMEAEDAATYYCQQRSRYPFTFGSGTKLEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 1048 - DNA, 3156 bp, synthetic construct
  misc_feature 1..3156: note = inhibitor receptor-T2A-activator receptor
  source 1..3156: mol_type = other DNA
caggtgcagctggtgcagtctggggctgaggtgaagaagcctgggtcctcagtgaaggtttcctgcaaggcttctggatacaccttcactagctatcatatacattgggtgcgccaggcccccggacaagggcttgagtggatcggatggatctaccctggcaatgttaacacagaatataatgagaagttcaagggcaaagccaccattaccgcggacgaatccacgaacacagcctacatggagctgagcagcctgagatctgaagacacggctgtgtattactgtgcgagggaggaaattacctacgctatggactactggggccagggaaccctggtcaccgtgtcctcaggcggaggtggaagcggagggggaggatctggcggcggaggaagcggaggcgacattcaaatgacccagagcccatccaccctgagcgcatctgtaggtgaccgggtcaccatcacttgtagatccagtcagagtattgtacacagtaatgggaacacctatttggaatggtatcagcagaaaccaggtaaagccccaaaattgctcatctacaaagtctctaacagatttagtggtgtaccagccaggttcagcggttccggaagtggtactgaattcaccctcacgatctcctctctccagccagatgatttcgccacttattactgttttcaaggttcacatgtgccgcgcacattcggtcagggtactaaagtagaagtcaaatacggctcacagagctccaaaccctacctgctgactcaccccagtgaccccctggagctcgtggtctcaggaccgtctgggggccccagctccccgacaacaggccccacctccacatctggccctgaggaccagcccctcacccccaccgggtcggatccccagagtggtctgggaaggcacctgggggttgtgatcggcatcttggtggccgtcatcctactgctcctcctcctcctcctcctcttcctcatcctccgacatcgacgtcagggcaaacactggacatcgacccagagaaaggctgatttccaacatcctgcaggggctgtggggccagagcccacagacagaggcctgcagtggaggtccagcccagctgccgatgcccaggaagaaaacctctatgctgccgtgaagcacacacagcctgaggatggggtggagatggacactcggagcccacacgatgaagacccccaggcagtgacgtatgccgaggtgaaacactccagacctaggagagaaatggcctctcctccttccccactgtctggggaattcctggacacaaaggacagacaggcggaagaggacaggcagatggacactgaggctgctgcatctgaagccccccaggatgtgacctacgcccagctgcacagcttgaccctcagacgggaggcaactgagcctcctccatcccaggaagggccctctccagctgtgcccagcatctacgccactctggccatccacggatccggagagggcagaggcagcctgctgacatgtggcgacgtggaagagaaccctggccccatggagtttgggctgagctggctttttcttgtggctattttaaaaggtgtccagtgtcaggtgcagctgagggagagcggccccagcctggtgaagcccagccagaccctgagcctgacctgcaccgtgagcggcttcagcctgaccagctacggcgtgcactgggtgaggcagccccccggcaagggcctggagtggctgggcgtgatctggaccggcggcaccaccaactacaacaccgccctgatgagcaggctgagcatcaccaaggacaacagcaagagccaggtgttcctgaagatgaacagcctgcagaccgacgacaccgccatctactactgcgccagggacggcgacagctacaacagggaggcctggttcgcctactggggccagggcaccctggtgaccgtgagcgccggcggaggtggaagcggagggggaggatctggcggcggaggaagcggaggccagatcgtgctgacccagagccccgccgtgatcagcgccagccccggcgagaaggtgatcctgacctgcagcgccagcagcagcgtgagctacatgcactggttccagcagaagcccggcaccagccccaagctgtggatctacggcaccagcaacctggccagcggcgtgcccgccaggttcagcggcggcggcagcggcaccagctacagcctgaccatcagcaggatggaggccgaggacgccgccacctactactgccagcagaggagcaggtaccccttcaccttcggcagcggcaccaagctggagatcaagaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttctgggtgctggtcgttgtgggcggcgtgctggcctgctacagcctgctggtgacagtggccttcatcatcttttgggtgaggagcaagcggagcagactgctgcacagcgactacatgaacatgaccccccggaggcctggccccacccggaagcactaccagccctacgcccctcccagggatttcgccgcctaccggagcaaacggggcagaaagaaactcctgtatatattcaaacaaccatttatgaggccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagcgtagaggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggactcagtacagccaccaaggacacctacgacgcccttcacatgcaggccctgccccctcgctag

SEQ ID NO: 1049 - protein, 1051 aa, synthetic construct
  REGION 1..1051: note = inhibitor receptor-T2A-activator receptor
QVQLVQSGAEVKKPGSSVKVSCKASGYTFTSYHIHWVRQAPGQGLEWIGWIYPGNVNTEYNEKFKGKATITADESTNTAYMELSSLRSEDTAVYYCAREEITYAMDYWGQGTLVTVSSGGGGSGGGGSGGGGSGGDIQMTQSPSTLSASVGDRVTITCRSSQSIVHSNGNTYLEWYQQKPGKAPKLLIYKVSNRFSGVPARFSGSGSGTEFTLTISSLQPDDFATYYCFQGSHVPRTFGQGTKVEVKYGSQSSKPYLLTHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIHGSGEGRGSLLTCGDVEENPGPMEFGLSWLFLVAILKGVQCQVQLRESGPSLVKPSQTLSLTCTVSGFSLTSYGVHWVRQPPGKGLEWLGVIWTGGTTNYNTALMSRLSITKDNSKSQVFLKMNSLQTDDTAIYYCARDGDSYNREAWFAYWGQGTLVTVSAGGGGSGGGGSGGGGSGGQIVLTQSPAVISASPGEKVILTCSASSSVSYMHWFQQKPGTSPKLWIYGTSNLASGVPARFSGGGSGTSYSLTISRMEAEDAATYYCQQRSRYPFTFGSGTKLEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 1050 - protein, 500 aa, synthetic construct; identical to SEQ ID NO: 1003
  REGION 1..500: note = murine HLA-A*02 inhibitor receptor

SEQ ID NO: 1051 - DNA, 1503 bp, synthetic construct
  misc_feature 1..1503: note = murine HAL-A*02 inhibitor receptor
  source 1..1503: mol_type = other DNA
gatgttttgatgacccaaactccactctccctgcctgtcagtcttggagatcaagcctccatctcttgcagatctagtcagagcattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagtagagtggaggctgaggatctgggagtttattactgctttcaaggttcacatgttcctcggacgtccggtggaggcaccaagctggaaatcaaaggcggaggtggaagcggagggggaggatctggcggcggaggaagcggaggccaggtccagctgcagcagtctggacctgagctggtgaagcctggggcttcagtgaggatatcctgcaaggcttctggctacaccttcacaagttaccatatacattgggtgaagcagaggcctggacagggacttgagtggattggatggatttatcctggaaatgttaatactgagtacaatgagaagttcaagggcaaggccacactgactgcagacaaatcgtccagcacagcctacatgcacctcagcagcctgacctctgaggactctgcggtctatttctgtgccagagaggagattacctatgctatggactactggggtcaaggaacctcagtcaccgtgtcctcatacggctcacagagctccaaaccctacctgctgactcaccccagtgaccccctggagctcgtggtctcaggaccgtctgggggccccagctccccgacaacaggccccacctccacatctggccctgaggaccagcccctcacccccaccgggtcggatccccagagtggtctgggaaggcacctgggggttgtgatcggcatcttggtggccgtcatcctactgctcctcctcctcctcctcctcttcctcatcctccgacatcgacgtcagggcaaacactggacatcgacccagagaaaggctgatttccaacatcctgcaggggctgtggggccagagcccacagacagaggcctgcagtggaggtccagcccagctgccgatgcccaggaagaaaacctctatgctgccgtgaagcacacacagcctgaggatggggtggagatggacactcggagcccacacgatgaagacccccaggcagtgacgtatgccgaggtgaaacactccagacctaggagagaaatggcctctcctccttccccactgtctggggaattcctggacacaaaggacagacaggcggaagaggacaggcagatggacactgaggctgctgcatctgaagccccccaggatgtgacctacgcccagctgcacagcttgaccctcagacgggaggcaactgagcctcctccatcccaggaagggccctctccagctgtgcccagcatctacgccactctggccatccactag

SEQ ID NO: 1052 - protein, 500 aa, synthetic construct
  REGION 1..500: note = humanized HLA-A*02 inhibitor receptor
QVQLVQSGAEVKKPGSSVKVSCKASGYTFTSYHIHWVRQAPGQGLEWIGWIYPGNVNTEYNEKFKGKATITADESTNTAYMELSSLRSEDTAVYYCAREEITYAMDYWGQGTLVTVSSGGGGSGGGGSGGGGSGGDIQMTQSPSTLSASVGDRVTITCRSSQSIVHSNGNTYLEWYQQKPGKAPKLLIYKVSNRFSGVPARFSGSGSGTEFTLTISSLQPDDFATYYCFQGSHVPRTFGQGTKVEVKYGSQSSKPYLLTHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 1053 - DNA, 1500 bp, synthetic construct
  misc_feature 1..1500: note = humanized HLA-A*02 inhibitor receptor
  source 1..1500: mol_type = other DNA
caggtgcagctggtgcagtctggggctgaggtgaagaagcctgggtcctcagtgaaggtttcctgcaaggcttctggatacaccttcactagctatcatatacattgggtgcgccaggcccccggacaagggcttgagtggatcggatggatctaccctggcaatgttaacacagaatataatgagaagttcaagggcaaagccaccattaccgcggacgaatccacgaacacagcctacatggagctgagcagcctgagatctgaagacacggctgtgtattactgtgcgagggaggaaattacctacgctatggactactggggccagggaaccctggtcaccgtgtcctcaggaggtggcggctctggaggcggaggtagcggaggtggaggctctggtggcgacattcaaatgacccagagcccatccaccctgagcgcatctgtaggtgaccgggtcaccatcacttgtagatccagtcagagtattgtacacagtaatgggaacacctatttggaatggtatcagcagaaaccaggtaaagccccaaaattgctcatctacaaagtctctaacagatttagtggtgtaccagccaggttcagcggttccggaagtggtactgaattcaccctcacgatctcctctctccagccagatgatttcgccacttattactgttttcaaggttcacatgtgccgcgcacattcggtcagggtactaaagtagaagtcaaatacggctcacagagctccaaaccctacctgctgactcaccctagtgatcctctggagctcgtggtctcaggaccgtctggaggcccaagctctccgacaacaggccccacctccacatctggccctgaggaccagcccctcacacccaccgggtcggatcctcagagtggtctgggaagacacctgggagttgtgatcggcatcttggtggccgtcatcctactgctcctcctcctgctcctgctcttcctcatcctccgacatcgacgtcagggcaaacactggacatcgacccagagaaaggctgatttccaacatcctgcaggggctgtggggccagagcccacagacagaggcctgcagtggaggtccagcccagctgccgatgcccaggaagaaaacctctatgctgccgtgaagcacacacagcctgaggatggggtggagatggatactcggagcccacacgatgaagatccacaggcagtgacgtatgccgaggtgaaacactccagacctagaagggaaatggcctctcctccttccccactgtctggagagttcctggacacaaaggacagacaggcggaagaggacaggcagatggacactgaggctgctgcatctgaagctcctcaggatgtgacctacgcccagctgcacagcttgaccctcagacgggaggcaactgagcctcctccatcccaggaagggccctctccagctgtgcccagcatctacgccactctggccatccac

SEQ ID NO: 1054 - protein, 511 aa, synthetic construct
  REGION 1..511: note = anti-HLA-E activator receptor
QVQLRESGPSLVKPSQTLSLTCTVSGFSLTSYGVHWVRQPPGKGLEWLGVIWTGGTTNYNTALMSRLSITKDNSKSQVFLKMNSLQTDDTAIYYCARDGDSYNREAWFAYWGQGTLVTVSAGGGGSGGGGSGGGGSGGQIVLTQSPAVISASPGEKVILTCSASSSVSYMHWFQQKPGTSPKLWIYGTSNLASGVPARFSGGGSGTSYSLTISRMEAEDAATYYCQQRSRYPFTFGSGTKLEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 1055 - DNA, 1536 bp, synthetic construct
  misc_feature 1..1536: note = anti-HLA-E activator receptor
  source 1..1536: mol_type = other DNA
caggtgcagctgagggagagcggccccagcctggtgaagcccagccagaccctgagcctgacctgcaccgtgagcggcttcagcctgaccagctacggcgtgcactgggtgaggcagccccccggcaagggcctggagtggctgggcgtgatctggaccggcggcaccaccaactacaacaccgccctgatgagcaggctgagcatcaccaaggacaacagcaagagccaggtgttcctgaagatgaacagcctgcagaccgacgacaccgccatctactactgcgccagggacggcgacagctacaacagggaggcctggttcgcctactggggccagggcaccctggtgaccgtgagcgccggcggaggtggaagcggagggggaggatctggcggcggaggaagcggaggccagatcgtgctgacccagagccccgccgtgatcagcgccagccccggcgagaaggtgatcctgacctgcagcgccagcagcagcgtgagctacatgcactggttccagcagaagcccggcaccagccccaagctgtggatctacggcaccagcaacctggccagcggcgtgcccgccaggttcagcggcggcggcagcggcaccagctacagcctgaccatcagcaggatggaggccgaggacgccgccacctactactgccagcagaggagcaggtaccccttcaccttcggcagcggcaccaagctggagatcaagaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttctgggtgctggtcgttgtgggcggcgtgctggcctgctacagcctgctggtgacagtggccttcatcatcttttgggtgaggagcaagcggagcagactgctgcacagcgactacatgaacatgaccccccggaggcctggccccacccggaagcactaccagccctacgcccctcccagggatttcgccgcctaccggagcaaacggggcagaaagaaactcctgtatatattcaaacaaccatttatgaggccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagcgtagaggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggactcagtacagccaccaaggacacctacgacgcccttcacatgcaggccctgccccctcgctag

SEQ ID NO: 1056 - protein, 247 aa, synthetic construct; identical to SEQ ID NO: 63
  REGION 1..247: note = HLA-A*02 scFv

SEQ ID NO: 1057 - DNA, 741 bp, synthetic construct; identical to SEQ ID NO: 75
  misc_feature 1..741: note = HLA-A*02 scFv
  source 1..741: mol_type = other DNA

SEQ ID NO: 1058 - protein, 247 aa, synthetic construct; identical to SEQ ID NO: 67
  REGION 1..247: note = HLA-A*02 scFv

SEQ ID NO: 1059 - DNA, 741 bp, synthetic construct
  misc_feature 1..741: note = HLA-A*02 scFv
  source 1..741: mol_type = other DNA
caggtgcagctggtgcagtctggggctgaggtgaagaagcctgggtcctcagtgaaggtttcctgcaaggcttctggatacaccttcactagctatcatatacattgggtgcgccaggcccccggacaagggcttgagtggatcggatggatctaccctggcaatgttaacacagaatataatgagaagttcaagggcaaagccaccattaccgcggacgaatccacgaacacagcctacatggagctgagcagcctgagatctgaagacacggctgtgtattactgtgcgagggaggaaattacctacgctatggactactggggccagggaaccctggtcaccgtgtcctcaggaggtggcggctctggaggcggaggtagcggaggtggaggctctggtggcgacattcaaatgacccagagcccatccaccctgagcgcatctgtaggtgaccgggtcaccatcacttgtagatccagtcagagtattgtacacagtaatgggaacacctatttggaatggtatcagcagaaaccaggtaaagccccaaaattgctcatctacaaagtctctaacagatttagtggtgtaccagccaggttcagcggttccggaagtggtactgaattcaccctcacgatctcctctctccagccagatgatttcgccacttattactgttttcaaggttcacatgtgccgcgcacattcggtcagggtactaaagtagaagtcaaa

(19) United States
(12) Patent Application Publication
(10) Pub. No.: US 2025/0177445 A1
(43) Pub. Date: Jun. 5, 2025

(54) ENGINEERED RECEPTORS SPECIFIC TO HLA-E AND METHODS OF USE

(71) Applicant: A2 Biotherapeutics, Inc., Agoura Hills, CA (US)

(72) Inventors: Carl Alexander Kamb, Thousand Oaks, CA (US); Han Xu, Agoura Hills, CA (US); Agnes Hamburger, Newbury Park, CA (US); Jing-Ping Hsin, Tarzana, CA (US); Jee Young Mock, North Hollywood, CA (US); Julyun Oh, Camarillo, CA (US); Yuta Ando, Agoura Hills, CA (US); Diane Manry, Agoura Hills, CA (US)

(21) Appl. No.: 18/844,372

(22) PCT Filed: Mar. 8, 2023
(86) PCT No.: PCT/US2023/063931

§ 371 Date: Sep. 5, 2024

Related U.S. Application Data
(60) Provisional application No. 63/323,858, filed on Mar. 25, 2022.
(60) Provisional application No. 63/317,714, filed on Mar. 8, 2022.
(60) Provisional application No. 63/484,324, filed on Feb. 10, 2023.

(51) Int. Cl. A61K 35/17; A61K 40/11; A61K 40/31; A61K 40/42; A61P 35/00; C07K 14/705; C07K 14/725; C07K 16/28; C12N 5/0783; C12N 15/86

(52) U.S. Cl. CPC A61K 35/17; A61K 40/11; A61K 40/31; A61K 40/4213; A61P 35/00; C07K 14/7051; C07K 14/70517; C07K 14/70521; C07K 16/2833; C12N 5/0636; C12N 15/86; A61K 2239/13; A61K 2239/17; A61K 2239/21; C07K 2317/53; C07K 2317/565; C07K 2317/622; C07K 2319/03; C07K 2319/33; C12N 2740/16043

(57) ABSTRACT

The disclosure relates to immune cells comprising a dual receptor system responsive to loss of heterozygosity in a target cell, and methods of making and using same. The first receptor comprises activator receptor specific to an HLA-E antigen, and the second receptor comprises an inhibitory receptor specific to an antigen lost in cancer but not wild type cells, that inhibits activation of the immune cells by the first receptor.

### CROSS-REFERENCE TO RELATED APPLICATIONS

The present application claims priority to U.S. Provisional Patent Application Ser. No. 63/317,714, filed Mar. 8, 2022, U.S. Provisional Patent Application Ser. No. 63/323,858, filed Mar. 25, 2022, and U.S. Provisional Patent Application Ser. No. 63/484,324, filed Feb. 10, 2023. The entire content of each of the above-referenced applications is incorporated herein by reference in its entirety.

### INCORPORATION BY REFERENCE OF SEQUENCE LISTING

The sequence listing paragraph application contains a Sequence Listing which has been submitted in .XML format ST26 via EFS-WEB and is hereby incorporated by reference in its entirety. Said .XML copy, created on Mar. 7, 2023 is named 061250-538001WO_SeqList_ST26.xml and is 1.43 MB in size.

### BACKGROUND

Cell and antibody-based therapies are a powerful tool for the treatment of various diseases, particularly cancers. In conventional adoptive cell therapies, immune cells are engineered to express specific receptors, for example chimeric antigen receptors (CARs) or T cell receptors (TCRs), which direct the activity of the immune cells to cellular targets via interaction of the receptor with an antigen expressed by the target cell. Identification of suitable target molecules remains challenging, as many targets are expressed in normal tissues as well as cancers. Human leukocyte antigen-E (HLA-E) is a subunit of the major histocompatibility complex (MHC) class I (MHC-I). HLA-E is overexpressed by many types of cancers, as well as being broadly expressed in normal tissues. There is thus a need in the art for compositions and methods useful in the treatment of diseases, particularly cancers, that affect cells that utilize the expression of HLA-E.

### SUMMARY

The disclosure provides immune cells expressing a two-receptor system. The first receptor comprises an activator receptor that activates the immune cells in response to an HLA-E activator antigen (sometimes referred to herein as an activator antigen). The second receptor inhibits immune cell activity in response to an inhibitory antigen.

The disclosure provides an immune cell, comprising (a) an activator receptor comprising an extracellular antigen binding domain specific to a human leukocyte antigen-E (HLA-E) antigen expressed by a cancer cell; and (b) an inhibitory receptor comprising an extracellular antigen binding domain specific to a non-target antigen that is not expressed by the cancer cell.

In some embodiments of the immune cells of the disclosure, expression of the non-target antigen is lost in the cancer cell. In some embodiments, expression of the non-target antigen is lost due to a loss of heterozygosity in the cancer cell.

In some embodiments of the immune cells of the disclosure, the extracellular antigen binding domain of the second receptor specifically binds an allelic variant of a major histocompatibility complex (MHC) protein. In some embodiments, the extracellular antigen binding domain of the second receptor specifically binds an allelic variant of an HLA-A, HLA-B, or HLA-C protein. In some embodiments, the extracellular antigen binding domain of the second receptor specifically binds to HLA-A*01, HLA-A*02, HLA-A*03, HLA-A*11, HLA-B*07, or HLA-C*07.

In some embodiments of the immune cells of the disclosure, the non-target antigen is encoded by an HLA class I allele or is a minor histocompatibility antigen (MiHA).

In some embodiments of the immune cells of the disclosure, the cancer cell expresses HLA-E. In some embodiments, the HLA-E*01 comprises HLA-E*01:01 or HLA-E*01:03. In some embodiments, the cancer cell is a colorectal renal cancer cell, an ovarian cancer cell, a cervical cancer cell, a melanoma cancer cell, a urothelial cancer cell, a pancreatic cancer cell, a gastric cancer cell, a head and neck cancer cell, a lung cancer cell, a breast cancer cell or a lymphoma cell.

In some embodiments of the immune cells of the disclosure, the non-target antigen is expressed by healthy cells of a subject. In some embodiments, healthy cells of the subject express both the target antigen and non-target antigen. In some embodiments, the activator receptor and the inhibitory receptor together can specifically activate the immune cell in the presence of the cancer cell.

In some embodiments of the immune cells of the disclosure, the immune cell is a T cell, an NK cell or a macrophage. In some embodiments, the T cell is a CD8+CD4− T cell. In some embodiments, the T cell is a CD8− CD4+ T cell.

In some embodiments of the immune cells of the disclosure, the activator receptor is a T cell receptor (TCR) or a chimeric antigen receptor (CAR).

In some embodiments of the immune cells of the disclosure, the extracellular antigen binding domain of the activator receptor comprises an antibody fragment, a single chain Fv antibody fragment (scFv), a β chain variable domain (Vβ), or a TCR α chain variable domain and a TCR β chain variable domain. In some embodiments, the extracellular antigen binding domain of the activator receptor is an scFv. In some embodiments, the extracellular antigen binding domain of the activator receptor comprises a heavy chain variable (VH) region and a light chain variable (VL) region. In some embodiments, the VH region comprises complement determining regions (CDRs) sequences of GFSLTSY (SEQ ID NO: 4), WTGGT (SEQ ID NO: 5) and DGDSYNREAWFAY (SEQ ID NO: 6), or sequences having at most 1, 2, or 3 substitutions, deletions, or insertions relative to SEQ ID NOS: 4-6. In some embodiments, the VL region comprises CDRs of SASSSVSYMH (SEQ ID NO: 1), GTSNLAS (SEQ ID NO: 2) and QQRSRYPFT (SEQ ID NO: 3), having at most 1, 2, or 3 substitutions, deletions, or insertions relative to SEQ ID NOS: 1-3. In some embodiments, the VH region comprises a sequence of QVQLRESGPSLVKPSQTLSLTCTVSGFSLTSYGVHWVRQPPGKGLEWLGVIWTGG TTNYNTALMSRLSITKDNSKSQVFLKMNSLQTDDTAIYYCARDGDSYNREAWFA YWGQGTLVTVSA (SEQ ID NO: 8), or a sequence having at least 90%, at least 95%, at least 96%, at least 97%, at least 98%, or at least 99% identity thereto. In some embodiments, the VL region comprises a sequence of QIVLTQSPAVISASPGEKVILTCSASSSVSYMHWFQQKPGTSPKLWIYGTSNLASG VPARFSGGGSGTSYSLTISRMEAEDAATYYCQQRSRYPFTFGSGTKLEIK (SEQ ID NO: 7), or a sequence having at least 90%, at least 95%, at least 96%, at least 97%, at least 98%, or at least 99% identity thereto.

In some embodiments of the immune cells of the disclosure, the CAR comprises a hinge sequence isolated or derived from CD8, CD28, IgG1, or IgG4, or a synthetic hinge. In some embodiments, the CAR comprises a transmembrane domain isolated or derived from CD8 or CD28. In some embodiments, the CAR comprises an intracellular domain isolated or derived from CD28, 4-1BB or CD3z, or a combination thereof.

In some embodiments of the immune cells of the disclosure, the inhibitory receptor comprises a TCR or CAR.

In some embodiments of the immune cells of the disclosure, the extracellular antigen binding domain of the inhibitory receptor comprises an antibody fragment, a single chain Fv antibody fragment (scFv), a β chain variable domain (Vβ), or a TCR α chain variable domain and a TCR β chain variable domain. In some embodiments, the extracellular antigen binding domain of the inhibitory receptor comprises a heavy chain variable (VH) region and a light chain variable (VL) region. In some embodiments, the VH and VL regions comprise CDRs selected from the group of CDR sequences disclosed in Table 5, or CDR sequences having at most 1, 2, or 3 substitutions, deletions, or insertions relative to thereto. In some embodiments, the inhibitory receptor comprises an extracellular antigen binding domain selected from the group of sequences disclosed in Tables 3 and 4, or a sequence having at least 85%, at least 90%, at least 95%, at least 97%, or at least 99% identity thereto.

In some embodiments of the immune cells of the disclosure, the VH region comprises (a) a heavy chain CDR1 selected from the group consisting of SGGYYWS (SEQ ID NO: 10), TSGVGVG (SEQ ID NO: 11), SYAMH (SEQ ID NO: 12), SYDMH (SEQ ID NO: 13), and SYWMH (SEQ ID NO: 14); (b) a HC CDR2 sequence selected from the group consisting of YIYYSGSTYYNPSLKS (SEQ ID NO: 15), LIYWNDDKRYSPSLKS (SEQ ID NO: 16), WINAGNGNTKYSQKFQG (SEQ ID NO: 17), AIGTAGDTYYPGSVKG (SEQ ID NO: 18), and RINSDGSSTSYADSVKG (SEQ ID NO: 19); and (c) a HC CDR3 sequence selected from the group consisting of HYYYYSMDV (SEQ ID NO: 20), HYYYYYLDV (SEQ ID NO: 21), HYYYYMDV (SEQ ID NO: 22), HYYYYYMDV (SEQ ID NO: 23), KTTSFYFDY (SEQ ID NO: 24), RHMRLSCFDY (SEQ ID NO: 25), EGNGANPDAFDI (SEQ ID NO: 26), DLPGSYWYFDL (SEQ ID NO: 27), and GVLLYNWFDP (SEQ ID NO: 28). In some embodiments, the VL region comprises a LC CDR1 comprising a sequence of RASQSISSYLN (SEQ ID NO: 29), a LC CDR2 comprising a sequence of AASSLQS (SEQ ID NO: 30) and a LC CDR3 comprising a sequence of QQSYSTPLT (SEQ ID NO: 31). In some embodiments, the scFv comprises a sequence having at least 85%, at least 90%, at least 95%, at least 97%, at least 99% identity or is identical to a sequence selected from the group consisting of SEQ ID NOS: 52-60.

In some embodiments of the immune cells of the disclosure, the inhibitory receptor comprises a LILRB1 intracellular domain or a functional variant thereof. In some embodiments, the inhibitory receptor comprises LILRB1 hinge and transmembrane domains, or functional variants thereof. In some embodiments, the inhibitory receptor comprises a sequence having at least 85%, at least 90%, at least 95%, at least 97%, at least 99% identity or is identical to

(SEQ ID NO: 254)
YGSQSSKPYLLTHPSDPLELVVSGPSGGPSSPTTGPTSTSGPEDQPLTP
TGSDPQSGLGRHLGVVIGILVAVILLLLLLLLLFLILRHRRQGKHWTST
QRKADFQHPAGAVGPEPTDRGLQWRSSPAADAQEENLYAAVKHTQPEDG
VEMDTRSPHDEDPQAVTYAEVKHSRPRREMASPPSPLSGEFLDTKDRQA
EEDRQMDTEAAASEAPQDVTYAQLHSLTLRREATEPPPSQEGPSPAVPS
IYATLAIH.

In some embodiments of the immune cells of the disclosure, expression and/or function of an MHC Class I gene has been reduced or eliminated. In some embodiments, the MHC Class I gene is beta-2-microglobulin (B2M). In some embodiments, the immune cell further comprises a polynucleotide comprising an interfering RNA, the interfering RNA comprising a sequence complementary to a sequence of a B2M mRNA. In some embodiments, the immune cell comprises one or more modifications to a sequence encoding B2M, wherein the one or more modifications reduce the expression and/or eliminate the function of B2M. In some embodiments, the one or more modifications are introduced with a nucleic acid guided endonuclease in a complex with at least one guide nucleic acid (gNA) that specifically targets a sequence of the endogenous gene encoding B2M. In some embodiments, the MHC Class I gene is HLA-A*02. In some embodiments, the immune cell further comprises a polynucleotide comprising an interfering RNA, comprising a sequence complementary to a sequence of an HLA-A*02 mRNA. In some embodiments, the immune cell comprises one or more modifications to a sequence of an endogenous gene encoding HLA-A*02, wherein the one or modifications reduce the expression and/or eliminate the function of HLA-A*02. In some embodiments the one or more modifications are introduced with a nucleic acid guided endonuclease in a complex with at least one guide nucleic acid (gNA) that specifically targets a sequence of the endogenous gene encoding HLA-A*02.

In some embodiments of the immune cells of the disclosure, the immune cell is autologous. In some embodiments, the immune cell is allogeneic.

The disclosure provides a pharmaceutical composition comprising a therapeutically effective amount of the immune cells of the disclosure. In some embodiments, the pharmaceutical composition further comprises a pharmaceutically acceptable carrier, diluent or excipient.

The disclosure provides a pharmaceutical composition of the disclosure for use as a medicament in the treatment of cancer.

The disclosure provides a polynucleotide or polynucleotide system, comprising one or more polynucleotides comprising polynucleotide sequences encoding: (a) an activator receptor comprising an extracellular antigen binding domain specific to an human leukocyte antigen-E (HLA-E) antigen expressed by a cancer cell; and (b) an inhibitory receptor comprising an extracellular antigen binding domain specific to a non-target antigen that is not expressed by the cancer cell.

The disclosure provides a vector, comprising the polynucleotide or polynucleotide system of the disclosure.

The disclosure provides a method of selectively killing and/or inhibiting the proliferation of an HLA-E positive cell exhibiting loss-of-heterozygosity or loss of expression of a non-target antigen, comprising contracting the HLA-E positive cells with the immune cells of the disclosure.

The disclosure provides a method of treating an HLA-E positive cancer in a subject, comprising administering to the subject the immune cells of the disclosure.

The disclosure provides a method of making an immune cell therapy, comprising transforming immune cells with the polynucleotide, polynucleotide system, or vector of the disclosure.

The disclosure provides a kit comprising the immune cell or pharmaceutical composition of the disclosure.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A is a series of fluorescence activated cell sorting (FACS) plots showing HLA-E expression on HeLa cells. HLA-E was knocked out (KO) to generate an HLA-E null HeLa target cell line, which was then transfected with varying amounts of HLA-E mRNA.

FIG. 1B is a table (top) and pair of plots (bottom) showing activation of Jurkat cells by an HLA-E chimeric antigen receptor (CAR) when co-cultured with target cells expression the A antigen only (HLA-E expressing HeLa).

FIG. 2A is a series of FACS plots showing expression of HLA-A*02 on HeLa cells transfected with varying amounts of HLA-A*02 mRNA. HeLa cells are HLA-E positive and HLA-A*02 negative.

FIG. 2B is a table (top) and pair of plots (bottom) showing that activation of Jurkat cells expressing the HLA-E activator CAR C1024 is inhibited by an HLA-A*02 inhibitory receptor. NFAT-Luciferase Jurkat cells expressing the HLA-E activator CAR, either alone or in combination with an HLA-A*02 LIR1 inhibitory receptor (C1765), were co-cultured with HeLa cells (which are HLA-E positive) transfected with HLA-A*02 mRNA. HLA-A2 and HLA-A*02 are used interchangeably; RLU: relative luminescence units. The left plot shows the amount of HLA-A*02 mRNA used in transfection (X-axis) versus luminescence (Y-axis), the right plot shows HLA-A*02 molecules on HeLa cells as determined by Qifikit (X-axis) versus luminescence (Y-axis).

FIG. 3A is a series of plots showing expression of HLA-E on HeLa cells which HLA-E has been knocked out, and the cells have been transfected with 2000 ng to 0 ng of HLA-E mRNA, as indicated at left.

FIG. 3B is a pair of plots showing the percent (%) specific killing when primary T cells expressing either the HLA-E CAR, or the HLA-E CAR in combination with an HLA-A*02 LIR1 inhibitory receptor, were co-cultured with HeLa expressing variable amounts of HLA-E. Percent (%) specific killing is shown on the y-axis. HLA-E mRNA used in HeLa cell transfection is shown on the x-axis (left plot), or number of HLA-E molecules expressed by HeLa cells (x-axis, right plot).

FIG. 4A is a series of plots showing expression of HLA-A*02 on HeLa cells which have been transfected with 2000 ng to 0 ng of HLA-A*02 mRNA, as indicated at left.

FIG. 4B is a pair of plots showing the percent (%) specific killing when primary T cells expressing either the HLA-E CAR, or the HLA-E CAR in combination with an HLA-A*02 LIR1 inhibitory receptor, were co-cultured with HeLa expressing variable amounts of HLA-A*02 in the presence of a constant level of HLA-E. Percent (%) specific killing is shown on the y-axis. HLA-A*02 mRNA used in HeLa cell transfection is shown on the x-axis (left plot), or number of HLA-A*02 molecules expressed by HeLa cells (x-axis, right plot).

FIGS. 5A-5D are a series of plots that show Jurkat NFAT luciferase inhibition in Jurkat cells expressing an HLA-E activator CAR and an HLA-A*03 LIR1 inhibitory receptor (FIGS. 5A-5B), an HLA-A*11:01 LIR1 inhibitor receptor (FIG. 5C) or an HLA-B*07 inhibitor receptor (FIG. 5D). Jurkat cells were co-cultured with HeLa cells transfected with HLA-A*03 (FIGS. 5A-5B), HLA-A*11 (FIG. 5C) or HLA-B*07 (FIG. 5D) mRNA, and the relative luminescence was assessed after 6 hours.

FIG. 6A is a set of histograms showing expression of FLAG-epitope tagged HLA-A*03:01 mRNA titrated in HeLa cells, which was detected with anti-A*03:01 antibody.

FIG. 6B is a set of histograms showing expression of FLAG-epitope tagged HLA-A*11:01 mRNA titrated in HeLa cells, detected with anti-A*11:01 antibody.

FIG. 6C is a set of histograms showing expression of FLAG-epitope tagged HLA-B*07:02 mRNA titrated in HeLa cells, detected with anti-B*07:02 antibody (BB7.1).

FIG. 7A is a graph showing expression of HLA-E in tumor and normal tissues.

FIG. 7B is a plot showing correlation with HLA-E and HLA-A*02 mRNA expression.

FIG. 8 is a standard curve showing correlation with HLA-E (TPM) and HLA-E (molecules/cell).

FIG. 9 is a set of histograms showing expression of HLA-E CAR and Tmod in primary T cells.

FIG. 10A is a plot showing the percent (%) specific killing when HLA-E knocked out HeLa cells were treated with HLA-E dose-dependently. Percent (%) specific killing is shown on the y-axis. Number of HLA-E molecules expressed by HeLa cells is shown on the x-axis. A series of plots showing expression of HLA-E are shown on left.

FIG. 10B is a plot showing the percent (%) specific killing when HLA-A*02 knocked out HeLa cells were treated with HLA-A*02 dose-dependently. Percent (%) specific killing is shown on the y-axis. Number of HLA-A*02 molecules expressed by HeLa cells is shown on the x-axis. A series of plots showing expression of HLA-A*02 are shown on left.

FIG. 11 is a table showing the summary of sentivity results for Jurkat and primary T cell mRNA titration assay.

FIG. 12 is a plot showing the percent (%) specific killing when Target A (HLA-A(+) A*02(−)) or Target AB (HLA-A(+) A*02(+)) cells were co-cultured with HLA-E CAR or HLA-E Tmod cells. Percent (%) specific killing is shown on the y-axis. E:T ratio is shown on the x-axis.

FIG. 13A is a set of plots showing the percent (%) specific killing when Target A (HLA-E(+) A*02(−)) or Target AB (HLA-E(+) A*02(+)) cells were co-cultured with HLA-E CAR or HLA-E Tmod cells in various donor cells (D12333 is A*02(+); D163840 and D316069 are A*02(−)). Percent (%) specific killing is shown on the y-axis. E:T ratio is shown on the x-axis.

FIG. 13B is a table showing the summary of tumor vs. normal selectivity of HLA-E CAR and Tmond cells. TvN: tumor vs. normal; KO: knockout via CRSPR/Cas9; KD: knockdown via shRNA; CD56(−)CD4/8(+) are a mixture of T cells selected as CD56(−)CD4(+) or CD56(−)CD8(+).

FIG. 14 is a plot showing percent (%) specific killing when Target A (HLA-E(+) A*02(−)) and Target AB (HLA-E(+) A*02(+)) cells were mixed before they were co-cultured with HLA-E CAR or HLA-E Tmod cells. Percent (%) specific killing is shown on the y-axis. Donors are shown on the x-axis. E:T ratio was 1:1 for donors 1, 2, and 3, and 3:1 for Donor 4. Donors 1, 3, and 4 are A*02(+), and Donor 2 is A*02(−). B2M was knocked out by CRISPR in donors 1 and 2. B2M was knocked down by shRNA in donors 3 and 4.

FIG. 15A is a set of plots showing percent (%) specific killing when A375 Target A (HLA-E(+) A*02(−)) or Target AB (HLA-E(+) A*02(+)) cells were co-cultured with HLA-E CAR or HLA-E Tmod with the B2M shRNA module cells. E:T ratio is 1:1. Percent (%) specific killing is shown on the y-axis. Hours are shown on the x-axis. A375 cells were used as target cells.

FIG. 15B is a set of plots showing the percent (%) specific killing when D328844 (HLA-A*02(+)) donor cells were used. Percent (%) specific killing is shown on the y-axis. Hours are shown on the x-axis. A375 cells were used as target cells.

FIG. 16A is a schematic diagram showing the dual-flank design of in vivo xenograft experiment.

FIG. 16B is a series of plots showing the graft volume (mm²) over time post implant in “normal” (HLA-E(+) A*02(+)) on the right flank and “tumor” (HLA-E(+) A*02(−)) on the left flank. Dotted line indicates day of T-cell injection via tail vein. The graft volume (mm²) is shown on the y-axis. Days post graft implantation are shown on the x-axis.

FIG. 17A is a schematic diagram showing surrogate Tmod targeting mouse orthologs allow readout of whole-body responses to CAR and Tmod cells.

FIG. 17B is a set of histograms showing expression of surrogate Tmod activator and blocker.

FIG. 17C is a plot showing maximum percent (%) specific killing in E:T dose-response with MS751 target cells. Percent (%) specific killing is shown on the y-axis. E:T ratio is shown on the x-axis.

FIG. 18 is a series of plots showing the percent (%) survival (top) and T cells counts (bottom). Different doses of T cells were indicated in each column. Percent (%) specific killing is shown on the y-axis (top). Number of hCD3+ cells/μl blood of mice is shown on the y-axis (bottom). Days post graft implantation are shown on the x-axis (top and bottom).

FIG. 19A is a table showing H-2K^{d}/D^{b} Tmod cells do not induce a clinical observation.

FIG. 19B is a table showing clinical observation codes and severity codes used in FIG. 19A.

FIG. 20 is a plot showing Tmod+ cells (%). Tmod+ cells (%) is shown on the y-axis. Days are shown in the x-axis. Cells were CD56 depleted and CD4/8 enriched.

FIG. 21 is a series of plots showing the percent (%) specific killing in E:T dose-response at day 13 post transduction or day 28 post transduction. Percent (%) specific killing is shown on the y-axis. E:T ratio is shown on the x-axis. Cells were CD56 depleted and CD4/8 enriched.

FIG. 22A is a plot showing the percent (%) maximum specific killing when Target A (HLA-E(+) and A*02(−)) or Target AB (HLA-E(+) and A*02(+)) cells were co-cultured with HLA-E Tmod cells. Percent (%) specific killing is shown on the y-axis. E:T ratio is shown on the x-axis.

FIG. 22B is a plot showing the percent (%) maximum specific killing when Target A (HLA-E(+) and A*02(−)) or Target AB (HLA-E(+) and A*02(+)) cells were co-cultured with HLA-E Tmod cells over time at an E:T ratio of 3:1. Percent (%) specific killing is shown on the y-axis. Time in hours is shown on the x-axis.

FIG. 22C is a plot showing the percent (%) maximum specific killing when Target A (HLA-E(+) and A*02(−)) or Target AB (HLA-E(+) and A*02(+)) cells were co-cultured with HLA-E Tmod cells over time at an E:T ratio of 1:1. Percent (%) specific killing is shown on the y-axis. Time in hours is shown on the x-axis.

### DETAILED DESCRIPTION

Provided herein are compositions and methods for treating cancers using immune cells comprising a two-receptor system responsive to differences in gene expression of a ligand between cancer and normal, wild type cells. These differences in expression can be due to loss of heterozygosity in the cancer cells. Alternatively, the differences in expression can be because the gene expression is not expressed in cancer cells, or is expressed in cancer cells at a lower level than normal cells. The two-receptor system is expressed in immune cells, for example immune cells used in adoptive cell therapy, and targets activity of these immune cells to cancer cells exhibiting loss of heterozygosity or expression differences. In this two-receptor system, the first receptor (an activator receptor, sometimes referred to herein as an A module) activates, or promotes activation of the immune cells, while the second receptor (an inhibitory receptor, sometimes referred to herein as a blocker, or inhibitor receptor, or a B module) acts to inhibit activation of the immune cells by the first receptor. Each receptor contains a ligand-binding domain (LBD) that binds a specific ligand. Signals from the two receptors upon ligand binding are integrated by the immune cell. Differential expression of ligands for the first and second receptors in cancer and normal cells, for example through loss of heterozygosity of the locus encoding the inhibitory ligand in cancer cells, or differences in transcription levels, mediates activation of immune cells by target cancer cells that express the first activator ligand but not the second inhibitory ligand.

In particular embodiments of the compositions and methods provided herein, immune cells comprising the two-receptor system described herein are used to treat human leukocyte antigen-E (HLA-E) positive cancers. This includes HLA-E-positive cancers such as colorectal cancer, renal cancer, ovarian cancer, cervical cancer, melanoma, urothelial cancer, pancreatic cancer, gastric, head and neck cancer, lung cancer, breast cancer or lymphoma. In the case of HLA-E-positive cancers, the target antigen of the activator receptor is an HLA-E antigen, which is frequently overexpressed in many types of cancers.

Heterozygous gene loss in a subset of tumors can be exploited to protect patients from on-target, off-tumor toxicity. By pairing an activator receptor with an inhibitory receptor, the methods provided herein increase the specificity of adoptive cell therapies and decrease harmful effects associated with these therapies, such as dose-limited toxicity. Immune cells comprising the HLA-E activator receptor and, for example, an HLA-A*02 specific inhibitory receptor can selectively kill HLA-A*02(−) tumor cells. Thus, an HLA-E activator receptor paired with an inhibitory receptor is a solid tumor therapeutic candidate whose activity is directed by a gene deleted in tumor cells such that normal tissue may be protected from HLA-E-mediated cytotoxicity.

An exemplary target for the second inhibitory receptor is expressed on the surface of normal cells that express HLA-E, and is lost from cancer cells through loss of heterozygosity (LOH) or other mechanisms, leaving a single allelic form in cancer cells that can be distinguished from other alleles via an allele-specific ligand binding domain on the inhibitory receptor. Exemplary targets of the inhibitory receptor include, but are not limited to, Major Histocompatibility Complex (MHC) proteins such as human leukocyte antigen-A (HLA-A). HLA-B, HLA-C, and other HLAs. HLAs are encoding by variant genes, such as HLA-A*01, HLA-A*02, HLA-A*03, HLA-C*07, and others, which can be lost from HLA-E positive cancer cells through loss of heterozygosity. Alternatively, further exemplary targets of the inhibitory receptor include, but are not limited to, minor histocompatibility antigens described below.

In variations, the compositions and methods described herein may be used to kill target cells and/or treat subjects in which expression of the non-target antigen is partially or completely decreased by causes other than loss of heterozygosity, including but not limited to partial gene deletion, epigenetic silencing, and point mutations or truncating mutations in the sequence encoding the non-target antigen.

The present disclosure describes engineered receptors, such as chimeric antigen receptors (CARs) and engineered T cell receptors (TCRs), adoptive cell therapies, and methods of use thereof. The engineered receptors can target the HLA-E antigen and activate immune cells genetically altered to express the HLA-E targeting receptor. Immune cells expressing the HLA-E targeting activator receptors can be used in compositions as part of cell adoptive therapies in the treatment, for example, of cancers that are HLA-E positive. The immune cells expressing the HLA-E targeting activator receptors are also contemplated to further comprise a second inhibitory receptor. The second inhibitory receptor can prevent or inhibit the activation of an immune cell mediated by the activator receptor. For example, an immune cell expressing an activator receptor targeting HLA-E and an inhibitory receptor that specifically binds a separate antigen, e.g. an HLA-A MHC Class I antigen, will be activated in the presence of a cell expressing HLA-E, but will not be activated in the presence of a cell expressing both HLA-E and the HLA-A antigen that is the target of the inhibitory receptor. Selective activation and inhibition of the immune cell expressing both an activator and inhibitory receptor is useful for reducing toxicity in adoptive cell therapies. The inventors have found that this strategy can be employed using the HLA-E-targeting activator receptors and inhibitory receptors described herein. Both activating and inhibitory receptors can be, for example, chimeric antigen receptors (CARs) or T cell receptors (TCRs), or other suitable receptor architectures comprising an inhibitory intracellular domain.

### Definitions

Prior to setting forth this disclosure in more detail, it may be helpful to an understanding thereof to provide definitions of certain terms to be used herein.

Unless defined otherwise, all technical and scientific terms used herein have the same meaning as commonly understood by those of ordinary skill in the art to which the disclosure belongs. Although any methods and materials similar or equivalent to those described herein can be used in the practice or testing of particular embodiments, preferred embodiments of compositions, methods and materials are described herein. For the purposes of the present disclosure, the following terms are defined below. Additional definitions are set forth throughout this disclosure.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length ±15%, ±10%, ±9%, ±8%, ±7%, ±6%, ±5%, ±4%, ±3%, ±2%, or ±1% about a reference quantity, level, value, number, frequency, percentage, dimension, size, amount, weight or length.

As used herein, the term “isolated” means material that is substantially or essentially free from components that normally accompany it in its native state. In particular embodiments, the term “obtained” or “derived” is used synonymously with isolated.

The terms “subject,” “patient” and “individual” are used interchangeably herein to refer to a vertebrate, preferably a mammal, more preferably a human. Tissues, cells, and their progeny of a biological entity obtained in vivo or cultured in vitro are also encompassed. A “subject,” “patient” or “individual” as used herein, includes any animal that exhibits pain that can be treated with the vectors, compositions, and methods contemplated herein. Suitable subjects (e.g., patients) include laboratory animals (such as mouse, rat, rabbit, or guinea pig), farm animals, and domestic animals or pets (such as a cat or dog). Non-human primates and, preferably, human patients, are included.

As used herein “treatment” or “treating,” includes any beneficial or desirable effect, and may include even minimal improvement in symptoms. “Treatment” does not necessarily indicate complete eradication or cure of the disease or condition, or associated symptoms thereof.

As used herein, “prevent,” and similar words such as “prevented,” “preventing” etc., indicate an approach for preventing, inhibiting, or reducing the likelihood of a symptom of disease. It also refers to delaying the onset or recurrence of a disease or condition or delaying the occurrence or recurrence of the symptoms of a disease. As used herein, “prevention” and similar words also include reducing the intensity, effect, symptoms and/or burden of disease prior to onset or recurrence.

As used herein, the term “amount” refers to “an amount effective” or “an effective amount” of a virus to achieve a beneficial or desired prophylactic or therapeutic result, including clinical results.

The term “therapeutically effective amount” refers to an amount that is effective to “treat” a subject (e.g., a patient). A therapeutically effective amount of an agent (e.g., a cell) may vary according to factors such as the disease state, age, sex, and weight of the individual, and the ability of the virus or cell to elicit a desired response in the individual. A therapeutically effective amount is also one in which any toxic or detrimental effects of the agent are outweighed by the therapeutically beneficial effects.

An “increased” or “enhanced” amount of a response, e.g., physiological activity or cellular activity, is typically a “statistically significant” amount, and may include an increase that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the level of activity in an untreated cell.

A “decreased” or “reduced” amount of a response, e.g., physiological activity or cellular activity, is typically a “statistically significant” amount, and may include an decrease that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the level of activity in an untreated cell.

By “maintain,” or “preserve,” or “maintenance,” or “no change,” or “no substantial change,” or “no substantial decrease” refers generally to a response that is comparable to a response caused by either vehicle, or a control composition. A comparable response is one that is not significantly different or measurable different from the reference response.

The term “chimeric antigen receptors” or “CARs” as used herein, may refer to artificial T-cell receptors, chimeric T-cell receptors, or chimeric immunoreceptors, for example, and encompass engineered receptors that graft an artificial specificity onto a particular immune effector cell, such as a helper T cell (CD4+), cytotoxic T cell (CD8+) or NK cell. CARs may be employed to impart the specificity of a monoclonal antibody onto a T cell, thereby allowing a large number of specific T cells to be generated, for example, for use in adoptive cell therapy. In specific embodiments, CARs direct specificity of the cell to a tumor associated antigen such as an HLA-E antigen. In some embodiments, CARs comprise an intracellular signaling domain, a transmembrane domain, and an extracellular domain comprising an antigen-binding region. In some embodiments, CARs comprise fusions of single-chain variable fragments (scFvs) or scFabs derived from monoclonal antibodies, fused to a transmembrane domain and intracellular signaling domain(s). The fusion may also comprise a hinge. Either heavy-light (H-L) and light-heavy (L-H) scFvs may be used. The specificity of CAR designs may be derived from antigens of receptors (e.g., peptides). Depending on the type of intracellular domain, a CAR can be an activator receptor or an inhibitory receptor. In some embodiments, for example when the CAR is an activator receptor, the CAR comprises domains for additional co-stimulatory signaling, such as CD3ζ, FcR, CD27, CD28, CD137, DAP10, and/or OX40. In some embodiments, molecules can be co-expressed with the CAR, including co-stimulatory molecules, reporter genes for imaging (e.g., for positron emission tomography), gene products that conditionally ablate the T cells upon addition of a pro-drug, homing receptors, cytokines, and cytokine receptors. As used herein, characteristics attributed to a chimeric antigen receptor may be understood to refer to the receptor itself or to a host cell comprising the receptor.

As used herein, a “TCR”, sometimes also called a “TCR complex” or “TCR/CD3 complex” refers to a protein complex comprising a TCR alpha chain, a TCR beta chain, and one or more of the invariant CD3 chains (zeta, gamma, delta and epsilon), sometimes referred to as subunits. The TCR alpha and beta chains can be disulfide-linked to function as a heterodimer to bind to peptide-MHC complexes. Once the TCR alpha/beta heterodimer engages peptide-MHC, conformational changes in the TCR complex in the associated invariant CD3 subunits are induced, which leads to their phosphorylation and association with downstream proteins, thereby transducing a primary stimulatory signal. In an exemplary TCR complex, the TCR alpha and TCR beta polypeptides form a heterodimer, CD3 epsilon and CD3 delta form a heterodimer, CD3 epsilon and CD3 gamma for a heterodimer, and two CD3 zeta form a homodimer.

The term “stimulation” refers to a primary response induced by binding of a stimulatory domain or stimulatory molecule (e.g., a TCR/CD3 complex) with its cognate antigen thereby mediating a signal transduction event, such as, but not limited to, signal transduction via the TCR/CD3 complex. Stimulation can mediate altered expression of certain molecules, and/or reorganization of cytoskeletal structures, and the like.

The term “stimulatory molecule” or “stimulatory domain” refers to a molecule or portion thereof that, when natively expressed by a T-cell, provides the primary cytoplasmic signaling sequence(s) that regulate activation of the TCR complex in a stimulatory way for at least some aspect of the T-cell signaling pathway. TCR alpha and/or TCR beta chains of wild type TCR complexes do not contain stimulatory domains and require association with CD3 subunits such as CD3 zeta to initiate signaling. In one aspect, the primary stimulatory signal is initiated by, for instance, binding of a TCR/CD3 complex with a major histocompatibility complex (MHC) bound to peptide, and which leads to mediation of a T-cell response, including, but not limited to, proliferation, activation, differentiation, and the like. One or more stimulatory domains, as described herein, can be fused to the intracellular portion of any one or more subunits of the TCR complex, including TCR alpha, TCR beta, CD3 delta, CD3 gamma and CD3 epsilon.

As used herein, a “domain capable of providing a stimulatory signal” refers to any domain that, either directly or indirectly, can provide a stimulatory signal that enhances or increases the effectiveness of signaling mediated by the TCR complex to enhance at least some aspect of T-cell signaling. The domain capable of providing a stimulatory signal can provide this signal directly, for example with the domain capable of providing the stimulatory signal is a primary stimulatory domain or co-stimulatory domain. Alternatively, or in addition, the domain capable of providing the stimulatory signal can act indirectly. For example, the domain can be a scaffold that recruits stimulatory proteins to the TCR, or provide an enzymatic activity, such as kinase activity, that acts through downstream targets to provide a stimulatory signal.

As used herein, “activation” of an immune cell or an immune cell that is “activated” is an immune cell that can carry out one or more functions characteristic of an immune response. These functions include proliferation, release of cytokines, and cytotoxicity, i.e. killing of a target cell. Activated immune cells express markers that will be apparent to persons of skill in the art. For example, activated T cells can express one or more of CD69, CD71, CD25 and HLA-DR. An immune cell expressing an activator receptor (e.g. a HLA-E CAR) can be activated by the activator receptor when it becomes responsive to the binding of the receptor to a target antigen (e.g. HLA-E) expressed by the target cell. A “target antigen” can also be referred to as an “activator antigen” and may be isolated or expressed by a target cell. Activation of an immune cell expressing an inhibitory receptor can be prevented when the inhibitory receptor becomes responsive to the binding of a non-target antigen (e.g. HLA-A*02), even when the activator receptor is bound to the target activator ligand. A “non-target antigen” can also be referred to as an “inhibitory ligand” or a “blocker”, and may be isolated or expressed by a target cell.

As used herein, a “domain capable of providing an inhibitory signal” refers to any domain that, either directly or indirectly, can provide an inhibitory signal that inhibits or decreases the effectiveness signaling mediated by the TCR complex. The domain capable of providing an inhibitory signal can reduce, or block, totally or partially, at least some aspect of T-cell signaling or function. The domain capable of providing an inhibitory signal can provide this signal directly, for example with the domain capable of providing the inhibitory signal provides a primary inhibitory signal. Alternatively, or in addition, the domain capable of providing the stimulatory signal can act indirectly. For example, the domain can recruit additional inhibitory proteins to the TCR, or can provide an enzymatic activity that acts through downstream targets to provide an inhibitory signal.

As used herein, “intracellular domain” refers to the cytoplasmic or intracellular domain of a protein, such as a receptor, that interacts with the interior of the cell, and carries out a cytosolic function. As used herein, “cytosolic function” refers to a function of a protein or protein complex that is carried out in the cytosol of a cell. For example, intracellular signal transduction cascades are cytosolic functions.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. As another example, a range such as 95-99% identity, includes something with 95%, 96%, 97%, 98% or 99% identity, and includes subranges such as 96-99%, 96-98%, 96-97%, 97-99%, 97-98% and 98-99% identity. This applies regardless of the breadth of the range.

In general, “sequence identity” or “sequence homology” refers to an exact nucleotide-to-nucleotide or amino acid-to-amino acid correspondence of two polynucleotides or polypeptide sequences, respectively. Techniques for determining sequence identity include determining the nucleotide sequence of a polynucleotide and/or determining the amino acid sequence encoded thereby and comparing these sequences to a second nucleotide or amino acid sequence. Two or more sequences (polynucleotide or amino acid) can be compared by determining their “percent identity.” The percent identity of two sequences, whether nucleic acid or amino acid sequences, is the number of exact matches between two aligned sequences divided by the length of the shorter sequence and multiplied by 100. Percent identity may also be determined, for example, by comparing sequence information using the advanced BLAST computer program, including version 2.2.9, available from the National Institutes of Health. The BLAST program is based on the alignment method of Karlin and Altschul, Proc. Natl. Acad. Sci. USA 87:2264-2268 (1990) and as discussed in Altschul, et al., J. Mol. Biol. 215:403-410 (1990); Karlin And Altschul, Proc. Natl. Acad. Sci. USA 90:5873-5877 (1993); and Altschul et al., Nucleic Acids Res. 25:3389-3402 (1997). Briefly, the BLAST program defines identity as the number of identical aligned symbols (generally nucleotides or amino acids), divided by the total number of symbols in the shorter of the two sequences. The program may be used to determine percent identity over the entire length of the proteins being compared. Default parameters are provided to optimize searches with short query sequences in, for example, with the blastp program. The program also allows use of an SEG filter to mask-off segments of the query sequences as determined by the SEG program of Wootton and Federhen, Computers and Chemistry 17:149-163 (1993). Ranges of desired degrees of sequence identity are approximately 80% to 100% and integer values therebetween. Typically, the percent identities between a disclosed sequence and a claimed sequence are at least 80%, at least 85%, at least 90%, at least 95%, or at least 98%.

As used herein, a “subsequence” refers to a length of contiguous amino acids or nucleotides that form a part of a sequence described herein. A subsequence may be identical to a part of a full length sequence when aligned to the full length sequence, or less than 100% identical to the part of the full length sequence to which it aligns (e.g., 90% identical to 50% of the full sequence, or the like).

As used herein, a “polynucleotide system” refers to one or more polynucleotides. The one or more polynucleotides may be designed to work in concert for a particular application, or to produce a desired transformed cell.

The term “exogenous” is used herein to refer to any molecule, including nucleic acids, protein or peptides, small molecular compounds, and the like that originate from outside the organism. In contrast, the term “endogenous” refers to any molecule that originates from inside the organism (i.e., naturally produced by the organism).

A polynucleotide is “operably linked” to another polynucleotide when it is placed into a functional relationship with the other polynucleotide. For example, a promoter or enhancer is operably linked to a coding sequence if it affects the transcription of the sequence. A peptide is “operably linked” to another peptide when the polynucleotides encoding them are operably linked, preferably they are in the same open reading frame.

A “promoter” is a sequence of DNA needed to turn a gene on or off. Promoters are located immediately upstream and/or overlapping the transcription start site, and are usually between about one hundred to several hundred base pairs in length.

The term “MOI” is used herein to refer to multiplicity of infection, which is the ratio of agents (e.g. viral particles) to infection targets (e.g. cells).

In the present description, any concentration range, percentage range, ratio range, or integer range is to be understood to include the value of any integer within the recited range and, when appropriate, fractions thereof (such as one tenth and one hundredth of an integer), unless otherwise indicated. The term “about”, when immediately preceding a number or numeral, means that the number or numeral ranges plus or minus 10%.

As used herein, a “target cell” refers to a cell that is targeted by an adoptive cell therapy. For example, a target cell can be cancer cell, which can be killed by the transplanted immune cells of the adoptive cell therapy. Target cells of the disclosure express a target antigen such as HLA-E, and do not express a non-target antigen.

As used herein, a “non-target cell” refers to a cell that is not targeted by an adoptive cell therapy. For example, in an adoptive cell targeting cancer cells, normal, healthy, non-cancerous cells are non-target cells. Some, or all, non-target cells in a subject may express both the target antigen and the non-target antigen. Non-target cells in a subject may express the non-target antigen irrespective of whether or not these cells also express the target antigen.

Polymorphism refers to the presence of two or more variants of a nucleotide sequence in a population. A polymorphism may comprise one or more base changes, an insertion, a repeat, or a deletion. A polymorphism includes e.g. a simple sequence repeat (SSR) and a single nucleotide polymorphism (SNP), which is a variation, occurring when a single nucleotide of adenine (A), thymine (T), cytosine (C) or guanine (G) is altered.

As used herein, “a non-target allelic variant” refers to an allele of a gene whose product is expressed by non-target cells, but is not expressed by target cells. For example, a non-target allelic variant is an allele of a gene that is expressed by normal, non-cancer cells of subject, but not expressed by cancer cells of the subject. The expression of the non-target allelic variant can be lost in the cancer cells by any mechanism, including, but not limited to, loss of heterozygosity, mutation, or epigenetic modification of the gene encoding the non-target allelic variant.

As used herein, “specific to” or “specifically binds to” when used with respect to a ligand binding domain, such as an antigen binding domain, refers to a ligand binding domain that has a high specificity for a named target. Antibody specificity can viewed as a measure of the goodness of fit between the ligand binding domain and the corresponding ligand, or the ability of the ligand binding domain to discriminate between similar or even dissimilar ligands. In comparison with specificity, affinity is a measure of the strength of the binding between the ligand binding domain and ligand, such that a low-affinity ligand binding domain binds weakly and high-affinity ligand binding domain binds firmly. A ligand binding domain that is specific to a target allele is one that can discriminate between different alleles of a gene. For example, a ligand binding domain that is specific to HLA-A*02 will not bind, or bind more weakly to other HLA-A alleles such as HLA-A*01 or HLA-A*03. The person of skill in the art will appreciate that a ligand binding domain can be said to be specific to a particular target, and yet still have low levels of binding to one or more additional targets that do not affect its function in the receptor systems described herein.

As used herein, a “target antigen,” whether referred to using the term antigen or the name of a specific antigen, refers to an antigen expressed by a target cell, such as a cancer cell. Expression of target antigen is not limited to target cells. Target antigens may be expressed by both cancer cells and normal, non-cancer cells in a subject.

As used herein, a “non-target antigen” (or “blocker antigen”) whether referred to using the term antigen or the name of a specific antigen, refers to an antigen that is expressed by normal, non-cancer cells and is not expressed in cancer cells. This difference in expression allows the inhibitory receptor to inhibit immune cell activation in the presence of non-target cells, but not in the presence of target cells.

As used herein, “affinity” refers to strength of binding of a ligand to a single ligand binding site on a receptor, for example an antigen for the antigen binding domain of any of the receptors described herein. Ligand binding domains can have a weaker interaction (low affinity) with their ligand, or a stronger interaction (high affinity).

K_{D}, or dissociation constant, is a type of equilibrium constant that measures the propensity of a larger object to separate reversibly into smaller components, such as, for example, when a macromolecular complex comprising receptor and its cognate ligand separates into the ligand and the receptor. When the K_{D} is high, it means that a high concentration of ligand is need to occupy the receptor, and the affinity of the receptor for the ligand is low. Conversely, a low K_{D} means that the ligand has a high affinity for the receptor.

As used herein, a receptor that is “responsive” or “responsive to” refers to a receptor comprising an intracellular domain, that when bound by a ligand (i.e. antigen) generates a signal corresponding to the known function of the intracellular domain. An activator receptor bound to a target antigen can generate a signal that causes activation of an immune cell expressing the activator receptor. An inhibitory receptor bound to a non-target antigen can generate an inhibitory signal that prevents or reduces activation of an immune cell expressing the activator receptor. Responsiveness of receptors, and their ability to activate or inhibit immune cells expressing the receptors, can be assayed by any means known in the art and described herein, including, but not limited to, reporter assays and cytotoxicity assays.

Receptor expression on an immune cell can be verified by assays that report the presence of the activator receptors and inhibitory receptors described herein. For example, a population of immune cells can be stained with a labeled molecule (e.g. a fluorophore labeled receptor-specific antibody or a fluorophore-labeled receptor-specific ligand), and quantified using fluorescence activated cell sorting (FACS) flow cytometry. This method allows a percentage of immune cells in a population of immune cells to be characterized as expressing an activator receptor, an inhibitory receptor, or both receptors. The ratio of activator receptor and inhibitory receptors expressed by the immune cells described herein can be determined by, for example, digital droplet PCR. These approaches can be used to characterize the population of cells for the production and manufacturing of the immune cells, pharmaceutical compositions, and kits described herein. For the immune cells, pharmaceutical compositions, and kits described herein, it is understood that a suitable percentage of immune cells expressing both an activator receptor and an inhibitory receptor is determined specifically for the methods described herein. For example, a suitable percentage of immune cells expressing both an activator receptor and in inhibitory receptor can be at least 50%, at least 55%, at least 60%, at least 65%, at least 70%, at least 75%, at least 80%, at least 85%, at least 90%, or at least 95%. For example, a suitable percentage of immune cells expressing both an activator receptor and an inhibitory receptor can be at most 50%, at most 55%, at most 60%, at most 65%, at most 70%, at most 75%, at most 80%, at most 85%, at most 90%, or at most 95%. For example, a suitable ratio of activator receptor and inhibitory receptor in an immune cell can be about 5:1, about 4:1, about 3:1, about 2:1, about 1:1, about 1:2, about 1:3, about 1:4, or about 1:5. It is understood that purification, enrichment, and/or depletion steps can be used on populations of immune cells to meet suitable values for the immune cells, pharmaceutical compositions, and kits described herein.

A responsive receptor expressed by the immune cells described herein can be verified by assays that measure the generation of a signal expected to be generated by the intracellular domain of the receptor. Reporter cell lines, such as Jurkat-Luciferase NFAT cells (Jurkat cells), can be used to characterize a responsive receptor. Jurkat cells are derived from T cells and comprise a stably integrated nuclear factor of activated T-cells (NFAT)-inducible luciferase reporter system. NFAT is a family of transcription factors required for immune cell activation, whose activation can be used as a signaling marker for T cell activation. Jurkat cells can be transduced or transfected with the activator receptors and/or inhibitory receptors described herein. The activator receptor is responsive to the binding of a ligand if the Jurkat cell expresses a luciferase reporter gene, and the level of responsiveness can be determined by the level of reporter gene expression. The presence of luciferase can be determined using any known luciferase detection reagent, such as luciferin. An inhibitory receptor is responsive to the binding of a ligand if, when co-expressed with an activator receptor in Jurkat cells, it prevents a normally responsive immune cell from expressing luciferase in response to the activator receptor. For example, the responsiveness of an inhibitory receptor can be determined and quantified in a Jurkat cell expressing both an activator and an inhibitor by observing the following: 1) the Jurkat cell expresses luciferase in the presence of activator receptor ligand and absence of inhibitory receptor ligand; and 2) luciferase expression in the Jurkat cell is reduced or eliminated in the presence of both an activator receptor ligand and an inhibitory receptor ligand. This approach can be used to determine the sensitivity, potency, and selectivity of activator receptors and specific pairs of activator receptors and inhibitory receptors. The sensitivity, potency, and selectivity can be quantified by EC50 or IC50 values using dose-response experiments, where an activator receptor ligand and/or inhibitory receptor ligand is titrated into a culture of Jurkat cells expressing an activator receptor or a specific pair of activator and inhibitory receptors. Alternatively, the EC50 and IC50 values can be determined in a co-culture of immune cells (e.g. Jurkat cells or primary immune cells) expressing an activator receptor or a specific pair of activator and inhibitory receptors and target cells expressing an increasing amount of an activator ligand or inhibitor ligand. An increasing amount of activator ligand or inhibitor ligand can be accomplished in the target cell by, for example, titration of activator ligand or inhibitor ligand encoding mRNA into target cells, or use of target cells that naturally express different levels of the target ligands.

Activation of the immune cells described herein that express an activator receptor or specific pairs of activator and inhibitory receptors can be further determined by assays that measure the viability of a target cell following co-incubation with the immune cells. The immune cells, sometimes referred to as effector cells, are co-incubated with target cells that express an activator receptor ligand, an inhibitory receptor ligand, or both an activator and inhibitory receptor ligand. Following co-incubation, viability of the target cell is measured using any method to measure viability in a cell culture. For example, viability can be determined using a mitochondrial function assay that uses a tetrazolium salt substrate to measure active mitochondrial enzymes. Viability can also be determined using imaging based methods. Target cells can express a fluorescent protein, such as green fluorescent protein or red fluorescent protein. Reduction in total cell fluorescence indicates a reduction in viability of the target cell. A reduction in viability of the target cell following incubation with immune cells expressing an activator receptor or a specific pair of activator and inhibitory receptors is interpreted as target cell-mediated activation of the immune cell. A measure of the selectivity of the immune cells can also be determined using this approach. The immune cell expressing a pair of activator and inhibitory receptors is selective if the following is observed: 1) viability is reduced in target cells expressing the activator receptor ligand but not the inhibitory receptor ligand; 2) viability is not reduced in target cells expressing both an activator receptor ligand and an inhibitory receptor ligand. From these measurements, a “specific killing” value can be derived that quantifies the percentage of immune cell activation based on the reduction in viability of target cell as a percentage of a negative control (immune cells that do not express an activator receptor). Further, from these measurements a “selectivity ratio” value can be derived that represents the ratio of the specific killing observed in target cells expressing an activator receptor ligand in the absence of inhibitory receptor ligand to the specific killing observed in target cells expressing both an activator receptor ligand and an inhibitory receptor ligand. This approach can be used to characterize the population of cells for the production and manufacturing of the immune cells, pharmaceutical compositions, and kits described herein.

A suitable specific killing value for the immune cells, pharmaceutical compositions, and kits can be, for example, the following criteria: 1) at least 40%, at least 50%, at least 60%, at least 70%, at least 80%, at least 90%, at least 95%, at least 97% or at least 99% specific killing following a 48 hour co-incubation of immune cells and target cells expressing activator receptor ligand in the absence of inhibitory receptor ligand; and 2) less than or equal to 40%, less than or equal to 35%, less than or equal to 30%, less than or equal to 25%, less than or equal to 20%, less than or equal to 15%, less than or equal to 10%, less than or equal to 5%, less than or equal to 3% or less than or equal to 1% specific killing of target cell expressing both an activator receptor ligand and an inhibitory receptor ligand.

As a further example, a suitable specific killing value for the immune cells, pharmaceutical compositions and kits can be the following criteria: 1) between 30% and 99%, between 40% and 99%, between 50% and 99%, between 55% and 95%, between 60% and 95%, between 60% and 90%, between 50% and 80%, between 50% and 70% or between 50% and 60% of target cells expressing the activator ligand but not the inhibitor ligand are killed; and 2), between 1% and 40%, between 3% and 40%, between 5% and 40%, between 5% and 30%, between 10% and 30%, between 15% and 30% or between 5% and 20% of target cells expressing the activator ligand and the inhibitor ligand are killed.

As a still further example, a suitable specific killing value for the immune cells, pharmaceutical compositions, and kits can be, for example, the following criteria: 1) at least 50% specific killing following a 48 hour co-incubation of immune cells and target cells expressing activator receptor ligand in the absence of inhibitory receptor ligand; and 2) less than or equal to 20% specific killing of target cell expressing both an activator receptor ligand and an inhibitory receptor ligand. As a further example, the immune cells are capable of killing at least 30%, at least 40%, at least 50%, at least 60%, at least 70%, at least 80%, at least 90%, at least 95%, at least 97% or at least 99% of target cells expressing the activator ligand and not the inhibitor ligand over a period of 6 hours, 12 hours, 18 hours, 24 hours, 30 hours, 36 hours, 42 hours, 48 hours, 54 hours, or 60 hours, while killing less than 40%, less than 30%, less than 20%, less than 10%, less than 5%, less than 3% or less than 1% of target cells expressing the activator and inhibitor ligands over the same time period.

A suitable specific killing value of the target cell expressing an activator ligand in the absence of an inhibitory ligand value for the immune cells, pharmaceutical compositions, and kits can be, for example, at least about 50% to at least about 95%. A suitable specific killing value of the target cell expressing an activator ligand in the absence of an inhibitory ligand value for the immune cells, pharmaceutical compositions, and kits can be, for example, at least about 50%, at least about 55%, at least about 60%, at least about 65%, at least about 70%, at least about 75%, at least about 80%, at least about 85%, at least about 90%, or at least about 95%. A suitable specific killing value of the target cell expressing an activator ligand in the absence of an inhibitory ligand value for the immune cells, pharmaceutical compositions, and kits can be, for example, at most about 50%, at most about 55%, at most about 60%, at most about 65%, at most about 70%, at most about 75%, at most about 80%, at most about 85%, at most about 90%, or at most about 95%. A suitable specific killing value of target cells expressing both an activator receptor ligand and an inhibitory receptor ligand for the immune cells, pharmaceutical compositions, and kits can be can be less than about 50%, less than about 45%, less than about 40%, less than about 35%, less than about 30%, less than about 25%, less than about 20%, less than about 15%, less than about 10%, or less than about 5%. The suitable specific killing value for the immune cells, pharmaceutical compositions, and kits can be can be determined following about 6 hours, about 12 hours, about 18 hours, about 24, about 30 hours, about 36 hours, about 42 hours, about 48 hours, about 54 hours, about 60 hours, about 66 hours, or about 72 hours of co-incubation of immune cells with target cells.

A suitable specific killing value of the target cell expressing an activator ligand in the absence of an inhibitory ligand value for the immune cells, pharmaceutical compositions, and kits can be, for example, at least about 50% to at least about 95%. A suitable specific killing value of the target cell expressing an activator ligand in the absence of an inhibitory ligand value for the immune cells, pharmaceutical compositions, and kits can be, for example, at least about 50%, at least about 55%, at least about 60%, at least about 65%, at least about 70%, at least about 75%, at least about 80%, at least about 85%, at least about 90%, or at least about 95%. A suitable specific killing value of the target cell expressing an activator ligand in the absence of an inhibitory ligand value for the immune cells, pharmaceutical compositions, and kits can be, for example, at most about 50%, at most about 55%, at most about 60%, at most about 65%, at most about 70%, at most about 75%, at most about 80%, at most about 85%, at most about 90%, or at most about 95%. A suitable specific killing value of target cells expressing both an activator receptor ligand and an inhibitory receptor ligand for the immune cells, pharmaceutical compositions, and kits can be can be less than about 50%, less than about 45%, less than about 40%, less than about 35%, less than about 30%, less than about 25%, less than about 20%, less than about 15%, less than about 10%, or less than about 5%. The suitable specific killing value for the immune cells, pharmaceutical compositions, and kits can be can be determined following about 6 hours, about 12 hours, about 18 hours, about 24, about 30 hours, about 36 hours, about 42 hours, about 48 hours, about 54 hours, about 60 hours, about 66 hours, or about 72 hours of co-incubation of immune cells with target cells.

As used herein, the term “functional variant” refers to a protein that has one or more amino-acid substitutions, insertions, or deletions as compared to a parental protein, and which retains one or more desired activities of the parental protein. A functional variant may be a fragment of the protein (i.e. a variant having N- and/or C-terminal deletions) that retain the one or more desired activities of the parental protein.

All publications and patents mentioned herein are hereby incorporated by reference in their entirety as if each individual publication or patent was specifically and individually indicated to be incorporated by reference. In case of conflict, the present application, including any definitions herein, will control. However, mention of any reference, article, publication, patent, patent publication, and patent application cited herein is not, and should not be taken as an acknowledgment, or any form of suggestion, that they constitute valid prior art or form part of the common general knowledge in any country in the world.

### Engineered Receptors

The disclosure provides an activator receptor and an inhibitory receptor, both receptors comprising an extracellular region, the extracellular region of the activator receptor comprising a first antigen binding domain capable of an HLA-E antigen and activating or promoting activation of the receptor, which promotes activation of effector cells expressing the receptor. The disclosure further provides an inhibitory receptor comprising a second antigen binding domain capable of binding a second antigen, wherein binding of the second antigen by the second antigen binding domain inhibits, reduces, or prevents activation of effector cells even in the presence of HLA-E antigen. The first antigen, sometimes referred to herein as an antigen, may also be referred to as activator antigen. The second antigen may also be referred to as inhibitor antigen. The activator and inhibitory receptors that bind to these antigens may also be referred to generally herein as engineered receptors. Engineered receptors can refer to either chimeric antigen receptors (CARs) and T cell receptors (TCRs) described in the disclosure. The term “engineered receptor” may also refer to any receptor designed using the binding domains, hinge regions, transmembrane domains, and/or cytoplasmic domains described herein. Engineered receptors of the disclosure comprise fusion proteins, and are sometimes referred to herein as fusion proteins.

### Activator Receptors

The disclosure provides a first HLA-E antigen, an activator, and a first engineered receptor comprising the first antigen binding domain that binds to the first activator antigen. In some embodiments, the first engineered receptor is an activator receptor. In some embodiments, the activator receptor is a T cell receptor (TCR) or a chimeric antigen receptor (CAR). In some embodiments, the first activator antigen is an HLA-E antigen. In some embodiments, the HLA-E antigen comprises a sequence or subsequence that shares at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or is identical to a sequence or a subsequence of SEQ ID NO: 291 or 292.

As used herein, an “activator” or “activator antigen” refers to a first antigen that binds to a first, activator antigen binding domain (LBD) of an engineered receptor of the disclosure, such as a CAR or TCR, thereby mediating activation of a T cell expressing the activator receptor. The activator antigen is expressed by target cells, for example cancer cells, and may also be expressed more broadly than just the target cells. For example the activator can be expressed on some, or all types of normal, non-target cells such as epithelial cells.

The activator receptors of the disclosure can specifically bind to an HLA-E antigen. HLA-E, also called HLA class I histocompatibility antigen, alpha chain E (HLA-E) and MHC class I antigen E, is a protein encoded by the major histocompatibility complex, class I, E (HLA-E) gene. In the MHC class I complex, HLA-E is part of a heterodimer, with HLA-E providing the alpha chain, or heavy chain, and β-2 microglobulin (B2M) providing the light chain. MHC class I complexes comprising HLA-E are involved in antigen presentation on the cell surface, and are known to play a role in cell recognition by natural killer (NK) cells. HLA-E binds peptides derived from signal peptides of classical MHC class I molecules, such as HLA-A, B, C, and G. HLA-E is also known to present pathogen derived antigens in some cases. HLA-E is expressed in a variety of normal cells, including endothelial cells, blood cells such as B-cells, T-cells, granulocytes, monocytes, macrophages (peripheral blood mononuclear cells), Kupffer cells, Hofbauer cells, melanocytes, intestinal epithelial cells, basal glandular cells, glandular cells, keratinocytes, enterocytes, alveolar cells, pancreatic cells, and pericytes such as Ito cells. HLA-E is widely expressed in normal tissues, including, but not limited to, the colon, intestine, heart, vasculature, kidney, liver, lung, liver, pancreas, prostate, rectum, skin, testis, and blood. HLA-E is frequently expressed by ovarian, cervical and renal cancers, among others. To date, 298 alleles of HLA-E have been discovered. However, most alleles of HLA-E are either rare, or encode non-functional proteins (Kanevisky et al. Int. J. Mol. Sci. 2019, 20: 5496). Two alleles of HLA-E, HLA-E*01:01 and HLA-E*01:03 described below, cover more than 99% of the world population. These two alleles are distributed worldwide in roughly equal proportion.

In some embodiments, the antigen is an HLA-E antigen. HLA-E antigens include the HLA-E polypeptide alone, or in complex with B2M. HLA-E antigens also include HLA-E polypeptide in a complex with both B2M and an additional peptide (i.e., a peptide antigen). HLA-E binding domains of the disclosure can bind HLA-E polypeptide alone, in complex with B2M, and in complex with B2M and a peptide antigen.

All HLA-E isoforms, and sequences and subsequences thereof that act as HLA-E antigens are envisaged as within the scope of the disclosure. An exemplary HLA-E polypeptide comprises an HLA-E*01:01 allele of HLA-E. An exemplary HLA-E*01:01 polypeptide comprises a sequence of

| (SEQ ID NO: 291) | |
|---|---|
| 1 | MVDGTLLLLL SEALALTQTW AGSHSLKYFH TSVSRPGRGE PRFISVGYVD DTQFVRFDND |
| 61 | AASPRMVPRA PWMEQEGSEY WDRETRSARD TAQIFRVNLR TLRGYYNQSE AGSHTLQWMH |
| 121 | GCELGPDRRF LRGYEQFAYD GKDYLTLNED LRSWTAVDTA AQISEQKSND ASEAEHQRAY |
| 181 | LEDTCVEWLH KYLEKGKETL LHLEPPKTHV THHPISDHEA TLRCWALGFY PAEITLTWQQ |
| 241 | DGEGHTQDTE LVETRPAGDG TFQKWAAVVV PSGEEQRYTC HVQHEGLPEP VTLRWKPASQ |
| 301 | PTIPIVGIIA GLVLLGSVVS GAVVAAVIWR KKSSGGKGGS YSKAEWSDSA QGSESHSL. |

A still further exemplary HLA-E polypeptide comprises an HLA-E*01:03 allele of HLA-E. An exemplary HLA-E*01:03 polypeptide comprises a sequence of

| (SEQ ID NO: 292) | |
|---|---|
| 1 | MVDGTLLLLL SEALALTQTW AGSHSLKYFH TSVSRPGRGE PRFISVGYVD DTQFVRFDND |
| 61 | AASPRMVPRA PWMEQEGSEY WDRETRSARD TAQIFRVNLR TLRGYYNQSE AGSHTLQWMH |
| 121 | GCELGPDGRF LRGYEQFAYD GKDYLTLNED LRSWTAVDTA AQISEQKSND ASEAEHQRAY |
| 181 | LEDTCVEWLH KYLEKGKETL LHLEPPKTHV THHPISDHEA TLRCWALGFY PAEITLTWQQ |
| 241 | DGEGHTQDTE LVETRPAGDG TFQKWAAVVV PSGEEQRYTC HVQHEGLPEP VTLRWKPASQ |
| 301 | PTIPIVGIIA GLVLLGSVVS GAVVAAVIWR KKSSGGKGGS YSKAEWSDSA QGSESHSL. |

In SEQ ID NOS: 291 and 292, the signal peptide is underlined.

All HLA-E alleles are envisaged as within the scope of the instant disclosure. In some embodiments, the HLA-E antigen comprises a sequence or subsequence of SEQ ID NO: 291 or 292. In some embodiments, the HLA-E antigen comprises a sequence or subsequence having at least 80%, at least 85%, at least 90%, at least 95%, at least 97%, at least 99% or is identical to SEQ ID NO: 291 and an R at position 128 of SEQ ID NO: 291. In some embodiments, the HLA-E antigen comprises a sequence or subsequence having at least 80%, at least 85%, at least 90%, at least 95%, at least 97%, at least 99% or is identical to SEQ ID NO: 292 and an G at position 128 of SEQ ID NO: 292.

Any cancer comprising a cancer cell expressing an HLA-E antigen is an HLA-E positive cancer. In some embodiments, the HLA-E positive cancer comprises colorectal cancer, renal cancer, ovarian cancer, cervical cancer, melanoma, urothelial cancer, pancreatic cancer, gastric, head and neck cancer, lung cancer, breast cancer or lymphoma.

In some embodiments, the activator antigen is expressed by target cells and is not expressed by non-target cells (i.e. normal cells not targeted by the adoptive cell therapy). In some embodiments, the target cells are cancer cells and the non-target cells are non-cancerous cells.

In some embodiments, the activator antigen has high cell surface expression on the target cells. This high cell surface expression confers the ability to deliver large activation signals. Methods of measuring cell surface expression will be known to the person of ordinary skill in the art and include, but are not limited to, immunohistochemistry using an appropriate antibody against the activator antigen, followed by microscopy or fluorescence activated cell sorting (FACS).

The activator antigen is present on all target cells. In some embodiments, the target cells are cancer cells—such as HLA-E+ cancer cells, e.g., in HLA-E+ ovarian cancer.

In some embodiments, the activator antigen is present on a plurality of target cells. In some embodiments, the target cells are cancer cells. In some embodiments, the activator antigen is present on at least 50%, at least 60%, at least 70%, at least 80%, at least 85%, at least 90%, at least 95%, at least 96%, at least 97%, at least 98%, at least 99%, at least 99.5% or at least 99.9% of target cells. In some embodiments, the activator antigen is present on at least 95% target cells. In some embodiments, the activator antigen is present on at least 99% target cells.

In some embodiments, the first, activator antigen is expressed by a plurality of target cells and a plurality of non-target cells. In some embodiments, the plurality of non-target cells expresses both the first, activator antigen and the second inhibitor antigen.

### HLA-E Antigen Binding Domains

The disclosure provides chimeric antigen receptors (CARs) comprising a polypeptide. In some embodiments, the polypeptide comprises an extracellular HLA-E antigen binding domain, and the CAR is an activator CAR. Suitable antigen-binding domains include, but are not limited to antigen-binding domains from antibodies, antibody fragments, scFv, antigen-binding domains derived from T cell receptors, and the like. All forms of antigen-binding domains known in the art are envisaged as within the scope of the disclosure.

An “extracellular domain”, as used herein, refers to the extracellular portion of a protein. For example, the TCR alpha and beta chains each comprise an extracellular domain, which comprise a constant and a variable region involved in peptide-MHC recognition. The “extracellular domain” can also comprise a fusion domain, for example of fusions between additional domains capable of binding to and targeting a specific antigen and the endogenous extracellular domain of the TCR subunit.

The term “antibody,” as used herein, refers to a protein, or polypeptide sequences derived from an immunoglobulin molecule, which specifically binds to an antigen. Antibodies can be intact immunoglobulins of polyclonal or monoclonal origin, or fragments thereof and can be derived from natural or from recombinant sources.

The terms “antibody fragment” and “antibody binding domain” refer to at least one portion of an antibody, or recombinant variants thereof that contains the antigen-binding domain, i.e., an antigenic determining variable region of an intact antibody that is sufficient to confer recognition and specific binding of the antibody fragment to a target, such as an antigen and its defined epitope. Examples of antibody fragments include, but are not limited to, Fab, Fab′, F(ab′)2, and Fv fragments, single-chain (sc)Fv (“scFv”) antibody fragments, linear antibodies, single domain antibodies (abbreviated “sdAb”) (either VL or VH), camelid VHH domains, and multi-specific antibodies formed from antibody fragments.

The term “scFv” refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single polypeptide chain, and wherein the scFv retains the specificity of the intact antibody from which it is derived.

“Heavy chain variable region” or “VH” (or, in the case of single domain antibodies, e.g., nanobodies, “VHH”) with regard to an antibody refers to the fragment of the heavy chain that contains three CDRs interposed between flanking stretches known as framework regions, these framework regions are generally more highly conserved than the CDRs and form a scaffold to support the CDRs.

“Light chain variable region” or “VL” with regard to an antibody refers to the fragment of the light chain that contains three CDRs interposed between flanking stretches known as framework regions, these framework regions are generally more highly conserved than the CDRs and form a scaffold to support the CDRs.

Unless specified, as used herein a scFv may have the VL and VH variable regions in either order, e.g., with respect to the N-terminal and C-terminal ends of the polypeptide, the scFv may comprise VL-linker-VH or may comprise VH-linker-VL.

The term “antibody light chain,” refers to the smaller of the two types of polypeptide chains present in antibody molecules in their naturally occurring conformations. Kappa (“K”) and lambda (“λ”) light chains refer to the two major antibody light chain isotypes.

The term “recombinant antibody” refers to an antibody that is generated using recombinant DNA technology, such as, for example, an antibody expressed by a bacteriophage or yeast expression system. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using recombinant DNA or amino acid sequence technology which is available and well known in the art.

The term “Vβ domain”, “Vβ-only domain”, “β chain variable domain” or “single variable domain TCR (svd-TCR)” refers to an antigen binding domain that consists essentially of a single T Cell Receptor (TCR) beta variable domain that specifically binds to an antigen in the absence of a second TCR variable domain. The Vβ-only domain engages antigen using complementarity-determining regions (CDRs). Each Vβ-only domain contains three complement determining regions (CDR1, CDR2, and CDR3). Additional elements may be combined provided that the Vβ domain is configured to bind the epitope in the absence of a second TCR variable domain.

In some embodiments, the extracellular antigen binding domain of the first activator receptor comprises an antibody fragment, a single chain Fv antibody fragment (scFv), or a β chain variable domain (Vβ).

In some embodiments, the extracellular antigen binding domain of the first receptor comprises a TCR α chain variable domain and a TCR β chain variable domain.

In some embodiments, for example those embodiments wherein the receptor comprises a first and a second polypeptide, the antigen-binding domain is isolated or derived from a T cell receptor (TCR) extracellular domain or an antibody.

Any type of ligand binding domain that can regulate the activity of a receptor in a ligand dependent manner is envisaged as within the scope of the instant disclosure. In some embodiments, the ligand binding domain is an antigen binding domain. Exemplary antigen binding domains include, inter alia, scFv, SdAb, Vβ-only domains, and TCR antigen binding domains derived from the TCR α and β chain variable domains.

Any type of antigen binding domain is envisaged as within the scope of the instant disclosure.

For example, the first extracellular antigen binding domain may be part of a contiguous polypeptide chain including, for example, a Vβ-only domain, a single domain antibody fragment (sdAb) or heavy chain antibodies HCAb, a single chain antibody (scFv) derived from a murine, humanized or human antibodies (Harlow et al., 1999, In: Using Antibodies: A Laboratory Manual, Cold Spring Harbor Laboratory Press, N.Y.; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426). In some aspects, the first extracellular ligand binding domain comprises an antigen binding domain that comprises an antibody fragment. In further aspects, the first extracellular ligand binding domain comprises an antibody fragment that comprises a scFv or an sdAb.

In some embodiments, the antigen binding domain of the activator and/or inhibitory receptor comprises an scFv. In some embodiments, the scFv comprises a VL and VH region joined by a linker. In some embodiments, the linker comprises a glycine serine linker, for example GGGGSGGGGSGGGGSGG (SEQ ID NO: 293) which is encoded by GGCGGAGGTGGAAGCGGAGGGGGAGGATCTGGCGGCGGAGGAAGCGGAGGC (SEQ ID NO: 21977. In some embodiments, the scFv further comprises a signal sequence at the N terminus of the scFv. Exemplary signal sequences include MDMRVPAQLLGLLLLWLRGARC (SEQ ID NO: 294), which is encoded by

(SEQ ID NO: 295)
ATGGACATGAGGGTCCCCGCTCAGCTCCTGGGGCTCCTGCTACTCTGGC
TCCGAGGTGCCAGATGT.

In some embodiments, the extracellular antigen binding domain of the activator receptor comprises an antibody fragment, a single chain Fv antibody fragment (scFv), a β chain variable domain (Vβ), or a TCR α chain variable domain and a TCR β chain variable domain. In some embodiments, the extracellular antigen binding domain of the activator receptor comprises a heavy chain variable (VH) region and a light chain variable (VL) region. In some embodiments, the VH and VL regions comprise complement determining regions (CDRs) selected from the group of CDRs disclosed in Table 1.

TABLE 1
Exemplary HLA-E complementarity determining regions (CDRs)
HLA-E Light Chain CDR Sequences

| CDR-L1 | CDR-L2 | CDR-L3 |
|---|---|---|
| SASSSVSYMH | GTSNLAS | QQRSRYPFT |
| (SEQ ID NO: 1) | (SEQ ID NO: 2) | (SEQ ID NO: 3) |

HLA-E Heavy Chain CDR Sequences

| CDR-H1 | CDR-H2 | CDR-H3 |
|---|---|---|
| GFSLTSY | WTGGT | DGDSYNREAWFAY |
| (SEQ ID NO: 4) | (SEQ ID NO: 5) | (SEQ ID NO: 6) |

In some embodiments, the HLA-E antigen binding domain comprises a variable heavy (VH) and variable light (VL) region. For example, the HLA-E antigen binding domain is an scFv, antibody or antibody fragment comprising a heavy chain and a light chain, the heavy and light chains comprising a VH and VL region.

In some embodiments, the VH region comprises one or more CDR sequences selected from the group consisting of GFSLTSY (SEQ ID NO: 4), WTGGT (SEQ ID NO: 5), and DGDSYNREAWFAY (SEQ ID NO: 6), and the VL region comprises one or more CDR sequences selected from the group consisting of SASSSVSYMH (SEQ ID NO: 1), GTSNLAS (SEQ ID NO: 2) and QQRSRYPFT (SEQ ID NO: 3).

In some embodiments, the VH region comprises CDR sequences of GFSLTSY (SEQ ID NO: 4), WTGGT (SEQ ID NO: 5), and DGDSYNREAWFAY (SEQ ID NO: 6), and the VL region comprises CDR sequences of SASSSVSYMH (SEQ ID NO: 1), GTSNLAS (SEQ ID NO: 2) and QQRSRYPFT (SEQ ID NO: 3).

In some embodiments, the VH region comprises one or more CDR sequences selected from the group consisting of SYGVH (SEQ ID NO: 21979), VIWTGGTTNYNTALMS (SEQ ID NO: 21980), and DGDSYNREAWFAY (SEQ ID NO: 6), and the VL region comprises one or more CDR sequences selected from the group consisting of SASSSVSYMH (SEQ ID NO: 1), GTSNLAS (SEQ ID NO: 2) and QQRSRYPFT (SEQ ID NO: 3).

In some embodiments, the VH region comprises CDR sequences of SYGVH (SEQ ID NO: 21979), VIWTGGTTNYNTALMS (SEQ ID NO: 21980), and DGDSYNREAWFAY (SEQ ID NO: 6), and the VL region comprises CDR sequences of SASSSVSYMH (SEQ ID NO: 1), GTSNLAS (SEQ ID NO: 2) and QQRSRYPFT (SEQ ID NO: 3).

Exemplary full length HLA-E VH and VL regions are shown in Table 2 below.

TABLE 2
HLA-E VH and VL sequences

| | |
|---|---|
| VL region | QIVLTQSPAVISASPGEKVILTCSASSSVSYMHWFQQKPGTS |
| (Protein | PKLWIYGTSNLASGVPARFSGGGSGTSYSLTISRMEAEDAA |
| Sequence) | TYYCQQRSRYPFTFGSGTKLEIK (SEQ ID NO: 7) |
| VL region (DNA | CAGATCGTGCTGACCCAGAGCCCCGCCGTGATCAGCGCC |
| Sequence) | AGCCCCGGCGAGAAGGTGATCCTGACCTGCAGCGCCAGC |
| | AGCAGCGTGAGCTACATGCACTGGTTCCAGCAGAAGCCC |
| | GGCACCAGCCCCAAGCTGTGGATCTACGGCACCAGCAAC |
| | CTGGCCAGCGGCGTGCCCGCCAGGTTCAGCGGCGGCGGC |
| | AGCGGCACCAGCTACAGCCTGACCATCAGCAGGATGGAG |
| | GCCGAGGACGCCGCCACCTACTACTGCCAGCAGAGGAGC |
| | AGGTACCCCTTCACCTTCGGCAGCGGCACCAAGCTGGAG |
| | ATCAAG (SEQ ID NO: 979) |
| VH region | QVQLRESGPSLVKPSQTLSLTCTVSGFSLTSYGVHWVRQPP |
| (Protein | GKGLEWLGVIWTGGTTNYNTALMSRLSITKDNSKSQVFLK |
| Sequence) | MNSLQTDDTAIYYCARDGDSYNREAWFAYWGQGTLVTVS |
| | A (SEQ ID NO: 8) |
| VH region | CAGGTGCAGCTGAGGGAGAGCGGCCCCAGCCTGGTGAAG |
| (DNA sequence) | CCCAGCCAGACCCTGAGCCTGACCTGCACCGTGAGCGGC |
| | TTCAGCCTGACCAGCTACGGCGTGCACTGGGTGAGGCAG |
| | CCCCCCGGCAAGGGCCTGGAGTFVGGCTGGGCGTGATCT |
| | GGACCGGCGGCACCACCAACTACAACACCGCCCTGATGA |
| | GCAGGCTGAGCATCACCAAGGACAACAGCAAGAGCCAG |
| | GTGTTCCTGAAGATGAACAGCCTGCAGACCGACGACACC |
| | GCCATCTACTACTGCGCCAGGGACGGCGACAGCTACAAC |
| | AGGGAGGCCTGGTTCGCCTACTGGGGCCAGGGCACCCTG |
| | GTGACCGTGAGCGCC(SEQ ID NO: 980) |
| VH region | QVQLRESGPSLVKPSQTLSLTCTVSGFSLTSYGVHWVRQPP |
| (Protein | GKGLEWLGVIWTGGTTNYNTALMSRLSITKDNSKSQVFLK |
| Sequence) | MNSLQTDDTAIYYCARDGDSYNREAWFAYWGQGTLVTVS |
| | A (SEQ ID NO: 8) |
| VH region | CAGGTGCAGCTGAGGGAGAGCGGCCCCAGCCTGGTGAAG |
| (DNA sequence) | CCCAGCCAGACCCTGAGCCTGACCTGCACCGTGAGCGGC |
| | TTCAGCCTGACCAGCTACGGCGTGCACTGGGTGAGGCAG |
| | CCCCCCGGCAAGGGCCTGGAGTGGCTGGGCGTGATCTGG |
| | ACCGGCGGCACCACCAACTACAACACCGCCCTGATGAGC |
| | AGGCTGAGCATCACCAAGGACAACAGCAAGAGCCAGGT |
| | GTTCCTGAAGATGAACAGCCTGCAGACCGACGACACCGC |
| | CATCTACTACTGCGCCAGGGACGGCGACAGCTACAACAG |
| | GGAGGCCTGGTTCGCCTACTGGGGCCAGGGCACCCTGGT |
| | GACCGTGAGCGCC (SEQ ID NO: 21978) |

In Table 2, CDR sequences are bold and underlined.

In some embodiments, the HLA-E antigen binding domain comprises a VL region comprising SEQ ID NO: 7, or a sequence having at least 90%, at least 95%, at least 97%, at least 98% or at least 99% identity thereto. In some embodiments, the VL region comprises CDR sequences of SASSSVSYMH (SEQ ID NO: 1), GTSNLAS (SEQ ID NO: 2) and QQRSRYPFT (SEQ ID NO: 3) and has at least 90%, at least 95%, at least 97%, at least 98% or at least 99% identity to SEQ ID NO: 7. In some embodiments, the HLA-E antigen binding domain comprises a VL region comprising SEQ ID NO: 7. In some embodiments, the HLA-E antigen binding domain comprises a VH region comprising SEQ ID NO: 8, or a sequence having at least 90%, at least 95%, at least 97%, at least 98% or at least 99% identity thereto. In some embodiments, the VH region comprises CDR sequences of GFSLTSY (SEQ ID NO: 4), WTGGT (SEQ ID NO: 5), and DGDSYNREAWFAY (SEQ ID NO: 6), and has at least 90%, at least 95%, at least 97%, at least 98% or at least 99% identity to SEQ ID NO: 8. In some embodiments, the VH region comprises CDR sequences of SYGVH (SEQ ID NO: 21979), VIWTGGTTNYNTALMS (SEQ ID NO: 21980), and DGDSYNREAWFAY (SEQ ID NO: 6), and has at least 90%, at least 95%, at least 97%, at least 98% or at least 99% identity to SEQ ID NO: 8. In some embodiments, the HLA-E antigen binding domain comprises a VH region comprising SEQ ID NO: 8. In some embodiments, the HLA-E antigen binding domain comprises SEQ ID NO: 7 and SEQ ID NO: 8. In some embodiments, the HLA-E antigen binding domain comprises an antibody fragment, a single chain Fv antibody fragment (scFv), a β chain variable domain (Vβ), or a TCR α chain variable domain and a TCR β chain variable domain.

In some embodiments, the HLA-E antigen binding domains of the disclosure are specific to more than one HLA-E allele. For example, HLA-E antigen binding domains of the disclosure are pan-HLA-E, binding to all HLA-E alleles. Alternatively, HLA-E antigen binding domains are specific to HLA-E*01:01 or HLA-E*01:03. As a further alternative, HLA-E antigen binding domains specifically bind to both HLA-E*01:01 and HLA-E*01:03 alleles of HLA-E.

### Inhibitor Receptors

The disclosure provides a second antigen, an inhibitor, and a second inhibitory receptor comprising a second antigen binding domain that binds to the inhibitor antigen. In some embodiments, the second, inhibitory antigen is an antigen, and the second inhibitory receptor comprises an antigen binding domain that specifically recognizes the second, inhibitory antigen. In some embodiments, the second, inhibitory receptor is a CAR. In some embodiments, the second, inhibitory receptor is a TCR. Additional suitable inhibitory receptor architectures are envisaged as within the scope of the instant disclosure, and are described below.

In some embodiments, the inhibitory receptor comprises an extracellular antigen binding domain specific to a non-target antigen that has been lost in a cancer cell, such as an allelic variant of a gene. The non-target allelic variant can be lost in the cancer cell through any mechanism, such as, without limitation, epigenetic changes that effect non-target allelic variant expression, mutations to the gene encoding the non-target allelic variant, disruption of cellular signaling that regulates expression of the non-target allelic variant, chromosome loss, partial or complete deletion of the genomic locus, gene silencing through modification of nucleic acids or heterochromatin, or loss of expression through other mechanisms. In variations of the compositions and methods disclosed herein, the cells or subject treated may exhibit a loss of expression of the non-target allelic variant because of non-genetic changes. Accordingly the disclosure provides compositions and methods for killing cells and/or treating subject lacking expression of the non-target antigen from any cause, including but not limited to, loss of heterozygosity.

The disclosure provides a second inhibitory receptor comprising an extracellular region, the extracellular region comprising a second antigen binding domain capable of specifically binding to a second antigen that inhibits activation of effector cells expressing the first and second receptors, wherein the effector cells are activated by binding of the first antigen to the first activator receptor.

As used herein an “inhibitor” or “inhibitor antigen” refers to a second antigen that binds to a second, antigen binding domain (inhibitor LBD) of an engineered receptor of the disclosure, but inhibits activation of an immune cell expressing the engineered receptor. The inhibitor is not expressed by the target cells. The inhibitor antigen is also expressed in a plurality of normal, non-target cells, including normal, non-target cells that express the activator antigen, thereby protecting these cells from the cytotoxic effects of the adoptive cell therapy. Without wishing to be bound by theory, inhibitor antigens can block activation of the effector cells through a variety of mechanisms. For example, binding of the inhibitor antigen to the inhibitor LBD can block transmission of a signal that occurs upon binding of the activator antigen to the activator LBD that would, in the absence of the inhibitor, lead to activation of the immune cell expressing the engineered receptors described herein.

Alternatively, or in addition, binding of the inhibitor antigen to the second engineered receptor can cause loss of cell surface expression the first, activator receptor from the surface of the immune cells comprising the two receptor system described herein. Without wishing to be bound by theory, it is thought that immune cell engagement of activator and inhibitor antigens on normal cells causes the inhibitory receptor to cause removal of nearby activator receptor molecules from the immune cell surface. This process locally desensitizes the immune cell, reversibly raising its activation threshold. Immune cells that engage only the activator antigen on a target cell cause local activation signals which are unimpeded by signals from the second, inhibitory receptor. This local activation increases until release of cytotoxic granules leads to target cell selective cell death. However, modulation of surface receptor expression levels may not be the only mechanism by which inhibitory receptors inhibit activation of immune cells by the first activator receptor. Without wishing to be bound by theory, other mechanisms may come into play, including, but not limited to, cross-talk between activator and inhibitory receptor signaling pathways.

In some embodiments, the second antigen is not expressed by the target cells, and is expressed by the non-target cells. In some embodiments, the target cells are cancer cells and the non-target cells are non-cancerous cells.

In some embodiments, the second, inhibitor antigen is an antigen and the second antigen binding domain comprises an scFv domain.

In some embodiments, the second, inhibitor antigen binding domain comprises a Vβ-only antigen binding domain.

In some embodiments, the second, inhibitor antigen binding domain comprises an antigen binding domain isolated or derived from a T cell receptor (TCR). For example, the second, inhibitor antigen binding domain comprises TCR α and β chain variable domains.

In some embodiments, the second, inhibitor antigen binding domain is an antigen binding domain. Suitable antigen-binding domains include, but are not limited to antigen-binding domains from antibodies, antibody fragments, scFv, antigen-binding domains derived from T cell receptors, and the like.

The disclosure provides a second receptor, comprising an extracellular antigen binding domain specific to a non-target antigen that has been lost in a cancer cell, such as an allelic variant of a gene. The non-target allelic variant can be lost in the cancer cell through any mechanism, such as, without limitation, epigenetic changes that effect non-target allelic variant expression, mutations to the gene encoding the non-target allelic variant, disruption of cellular signaling that regulates expression of the non-target allelic variant, chromosome loss, partial or complete deletion of the genomic locus, gene silencing through modification of nucleic acids or heterochromatin, or loss of expression through other mechanisms. In variations of the compositions and methods disclosed herein, the cells or subject treated may exhibit a loss of expression of the non-target allelic variant because of non-genetic changes. Accordingly the disclosure provides compositions and methods for killing cells and/or treating subject lacking expression of the non-target antigen from any cause, including but not limited to, loss of heterozygosity.

The non-target antigen can be a protein, or an antigen peptide thereof in a complex with a major histocompatibility complex class I (MHC-I), where the non-target antigen comprises a polymorphism. Because the non-target antigen is polymorphic, loss of a single copy of the gene encoding the non-target antigen, which may occur through loss of heterozygosity in a cancer cell, yields a cancer cell that retains the other polymorphic variant of gene, but has lost the non-target antigen. For example, a subject having HLA-A*02 and HLA-A*01 alleles at the HLA locus may have a cancer in which only the HLA-A*02 allele is lost. As another example, a subject having HLA-A*03 and HLA-A*01 alleles at the HLA locus may have a cancer in which only the HLA-A*03 allele is lost. In such a subject, the HLA-A*01 protein remains present, but is not recognized by the inhibitory receptor of immune cells encountering the cancer cell, because the inhibitor receptor is designed to be specific to the HLA-A*02, HLA-A*03, or other non-target antigen. In normal non-malignant cells, the HLA-A*02, HLA-A*03, or other non-target antigen is present and inhibits activation of the engineered immune cell. In cancer cells having loss of heterozygosity, the HLA-A*02, HLA-A*03, or other allelic variant is lost. Immune cells engineered to express the inhibitory receptor do not receive an inhibitory signal from the inhibitory receptor, as the inhibitory receptor only responds to the HLA-A*02, HLA-A*03, or other non-target antigen, which is absent on cancer cells. By this mechanism, the immune cell is selectively activated, and selectively kills, cancer cells expressing HLA-E but having lost HLA-A*02 (or another non-target antigen) due to loss-of-heterozygosity. HLA-A is used here as an example. Similar polymorphic variation occurs in the population at other MHC genes and in other non-MHC genes as well.

The non-target antigen can be a protein, or an antigen peptide thereof in a complex with a major histocompatibility complex class I (MHC-I), where the non-target antigen comprises a polymorphism. Because the non-target antigen is polymorphic, loss of a single copy of the gene encoding the non-target antigen, which may occur through loss of heterozygosity in a cancer cell, yields a cancer cell that retains the other polymorphic variant of gene, but has lost the non-target antigen. For example, a subject having HLA-A*02 and HLA-A*01 alleles at the HLA locus may have a cancer in which only the HLA-A*02 allele is lost. In such a subject, the HLA-A*01 protein remains present, but is not recognized by the inhibitory receptor of immune cells encountering the cancer cell, because the inhibitor receptor is designed to be specific to the HLA-A*02 (or other non-target antigen). In normal non-malignant cells, the HLA-A*02 (or other non-target antigen) is present and inhibits activation of the engineered immune cell. In cancer cells having loss of heterozygosity, the HLA-A*02 allelic variant (or other non-target antigen) is lost. Immune cells engineered to express the inhibitory receptor do not receive an inhibitory signal from the inhibitory receptor, as the inhibitory receptor only responds to the HLA-A*02 (or other non-target antigen), which is absent on cancer cells. By this mechanism, the immune cell is selectively activated, and selectively kills, cancer cells expressing HLA-E but having lost HLA-A*02 (or another non-target antigen) due to loss-of-heterozygosity. In some embodiments, the non-target antigen comprises a human leukocyte antigen A*03 allele (HLA-A*03). In some embodiments, the non-target antigen comprises a human leukocyte antigen A*11 allele (HLA-A*11).

HLA-A is used here as an example. Similar polymorphic variation occurs in the population at other MHC genes and in other non-MHC genes as well.

The disclosure provides an inhibitory receptor, such as an inhibitory CAR or TCR. In some embodiments, the inhibitory receptor comprises an extracellular antigen binding domain specific to a non-target antigen that is not expressed by cancer cells due to a loss of heterozygosity in the cancer cells. In some embodiments, the non-target antigen comprises an HLA class I allele or a minor histocompatibility antigen (MiHA). In some embodiments, the HLA Class I allele comprises HLA-A, HLA-B, or HLA-C. In some embodiments, the HLA class I allele comprises HLA-A*02. In some embodiments, the HLA class I allele comprises HLA-A*11. In some embodiments, the HLA-A*02 or HLA-A*11 non-target antigen is expressed by healthy cells of a subject.

In some embodiments, the inhibitory receptor comprises an extracellular antigen binding domain specific to a non-target antigen that is not expressed by the cancer cell due to a loss of heterozygosity in the cancer cell.

Alternatively, or in addition, expression of activator and inhibitor targets may be correlated, i.e. the two are expressed at similar levels on non-target cells.

In some embodiments, the second, inhibitor antigen is a peptide antigen. In some embodiments, the second, inhibitor antigen is a peptide antigen complexed with a major histocompatibility (MHC) class I complex (peptide MHC, or pMHC). Inhibitor antigens comprising peptide antigens complexed with pMHC comprising any of HLA-A, HLA-B, or HLA-C are envisaged as within the scope of the disclosure.

In some embodiments, the non-target antigen comprises a Major Histocompatibility Complex (MHC) protein. In some embodiments, the MHC is MHC class I. In some embodiments, the MHC class I protein comprises a human leukocyte antigen (HLA) protein. In some embodiments, the non-target antigen comprises an allele of an HLA Class I protein selected from the group consisting of HLA-A, HLA-B, HLA-C, or HLA-E. In some embodiments, the HLA-A allele comprises HLA-A*01, HLA-A*02, HLA-A*03 or HLA-A*11. In some embodiments, the HLA-B allele comprises HLA-B*07. In some embodiments, the HLA-C allele comprises HLA-C*07.

In some embodiments, the non-target antigen comprises HLA-A. In some embodiments, the non-target antigen comprises an allele of HLA-A. In some embodiments, the allele of HLA-A comprises HLA-A*01, HLA-A*02, HLA-A*03 or HLA-A*11. In some embodiments, the non-target antigen comprises HLA-A*69.

In some embodiments, the non-target antigen comprises an allele HLA-B. In some embodiments, the allele of HLA-B comprises HLA-B*11.

In some embodiments, the non-target antigen comprises an allele of HLA-C. In some embodiments, the HLA-C allele comprises HLA-C*07.

In some embodiment, the inhibitor antigen is encoded by a gene that is absent or polymorphic in many tumors.

Methods of distinguishing the differential expression of inhibitor antigens between target and non-target cells will be readily apparent to the person or ordinary skill in the art. For example, the presence or absence of inhibitor antigens in non-target and target cells can be assayed by immunohistochemistry with an antibody that binds to the inhibitor antigen, followed by microscopy or FACS, RNA expression profiling of target cells and non-target cells, or DNA sequencing of non-target and target cells to determine if the genomic locus of the inhibitor antigen comprises mutations in either the target or non-target cells.

### Alleles Lost Due to Loss of Heterozygosity (LOH)

Homozygous deletions in primary tumors are rare and small, and therefore unlikely to yield target B candidates. For example, in an analysis of 2218 primary tumors across 21 human cancer types, the top four candidates were cyclin dependent kinase inhibitor 2A (CDKN2A), RB transcriptional corepressor 1 (RB1), phosphatase and tensin homolog (PTEN) and N3PB2. However, CDKN2A (P16) was deleted in only 5% homozygous deletion across all cancers. Homozygous HLA-A deletions were found in less than 0.2% of cancers (Cheng et al., Nature Comm. 8:1221 (2017)). In contrast, deletion of a single copy of a gene in cancer cells due to loss of hemizygosity occurs far more frequently.

In some embodiments, the second, inhibitor antigen comprises an allele of a gene that is lost in target cells due to loss of heterozygosity. In some embodiments, the target cells comprise cancer cells. Cancer cells undergo frequent genome rearrangements, including duplication and deletions. These deletions can lead to the deletion of one copy of one or more genes in the cancer cells.

As used herein, “loss of heterozygosity (LOH)” refers to a genetic change that occurs at high frequency in cancers, whereby one of the two alleles is deleted, leaving a single mono-allelic (hemizygous) locus.

### HLA Class I Alleles

In some embodiments, the second, inhibitor antigen comprises an HLA class I allele. The major histocompatibility complex (MHC) class I is a protein complex that displays antigens to cells of the immune system, triggering immune response. The Human Leukocyte Antigens (HLAs) corresponding to MHC class I are HLA-A, HLA-B, HLA-C, HLA-E, HLA-F and HLA-G.

In some embodiments, the second, inhibitor antigen comprises an HLA class I allele. In some embodiments, the second, inhibitor antigen comprises an allele of HLA class I that is lost in a target cell through LOH. HLA-A is a group of human leukocyte antigens (HLA) of the major histocompatibility complex (MHC) that are encoded by the HLA-A locus. HLA-A is one of three major types of human MHC class I cell surface receptors. The receptor is a heterodimer comprising a heavy a chain and smaller β chain. The α chain is encoded by a variant of HLA-A, while the β chain (β2-microglobulin) is invariant. There are several thousand HLA-A variants, all of which fall within the scope of the instant disclosure.

In some embodiments, the second, inhibitor antigen comprises an HLA-B allele. The HLA-B gene has many possible variations (alleles). Hundreds of versions (alleles) of the HLA-B gene are known, each of which is given a particular number (such as HLA-B27).

In some embodiments, the second, inhibitor antigen comprises an HLA-C allele. HLA-C belongs to the HLA class I heavy chain paralogues. This class I molecule is a heterodimer consisting of a heavy chain and a light chain (beta-2 microglobulin). Over one hundred HLA-C alleles have been described.

In some embodiments, the HLA class I allele has broad or ubiquitous RNA expression.

In some embodiments, the HLA class I allele has a known, or generally high minor allele frequency.

In some embodiments, the HLA class I allele does not require a peptide-MHC antigen, for example when the HLA class I allele is recognized by a pan-HLA antigen binding domain.

In some embodiments, the second inhibitor antigen comprises an HLA-A allele. In some embodiments the HLA-A allele comprises HLA-A*02. Various single variable domains known in the art or disclosed herein that bind to and recognize HLA-A*02 are suitable for use in embodiments. Such scFvs include, for example and without limitation, the following mouse and humanized scFv antibodies that bind HLA-A*02 in a peptide-independent way shown in Tables 3 and 4 below (complementarity determining regions underlined):

TABLE 3
HLA-A*02 scFv Sequences and Inhibitory receptor Constructs

| Protein Sequence | DNA Sequence |
|---|---|
| MDMRVPAQLLGLLLLWLRGARCDVLMTQTPLSLPVSLGDQASISCRS | (SEQ ID NO: 208) |
| SQSIVHSNGNTYLEWYLQKPGQSPKLLIYKVSNRFSGVPDRFSGSGSG | |
| TDFTLKISRVEAEDLGVYYCFQGSHVPRTSGGGTKLEIKGGGGSGGG | |
| GSGGGGSGGQVQLQQSGPELVKPGASVRISCKASGYTFTSYHIHWVK | |
| QRPGQGLEWIGWIYPGNVNTEYNEKFKGKATLTADKSSSTAYMHLSS | |
| LTSEDSAVYFCAREEITYAMDYWGQGTSVTVSS (SEQ ID NO: 207) | |
| MDMRVPAQLLGLLLLWLRGARCQVQLVQSGAEVKKPGSSVKVSCK | (SEQ ID NO: 210) |
| ASGYTFTSYHIHWVRQAPGQGLEWIGWIYPGNVNTEYNEKFKGKATI | |
| TADESTNTAYMELSSLRSEDTAVYYCAREEITYAMDYWGQGTLVTV | |
| SSGGGGSGGGGSGGGGSGGDIQMTQSPSTLSASVGDRVTITCRSSQSI | |
| VHSNGNTYLEWYQQKPGKAPKLLIYKVSNRFSGVPARFSGSGSGTEF | |
| TLTISSLQPDDFATYYCFQGSHVPRTFGQGTKVEVK (SEQ ID NO: 209) | |
| MDMRVPAQLLGLLLLWLRGARCQVQLVQSGAEVKKPGSSVKVSCK | (SEQ ID NO: 212) |
| ASGYTFTSYHMHWVRQAPGQGLEWIGYIYPGNVNTEYNEKFKGKAT | |
| LTADKSTNTAYMELSSLRSEDTAVYFCAREEITYAMDYWGQGTLVT | |
| VSSGGGGSGGGGSGGGGSGGDVQMTQSPSTLSASVGDRVTITCSSSQ | |
| SIVHSNGNTYMEWYQQKPGKAPKLLIYKVSNRFSGVPDRFSGSGSGT | |
| EFTLTISSLQPDDFATYYCHQGSHVPRTFGQGTKVEVK (SEQ ID NO: | |
| 211) | |
| DVLMTQTPLSLPVSLGDQASISCRSSQSIVHSNGNTYLEWYLQKPGQS | (SEQ ID NO: |
| PKLLIYKVSNRFSGVPDRFSGSGSGTDFTLKISRVEAEDLGVYYCFQG | 22019) |
| SHVPRTSGGGTKLEIKGGGGSGGGGSGGGGSGGQVQLQQSGPELVKP | |
| GASVRISCKASGYTFTSYHIHWVKQRPGQGLEWIGWIYPGNVNTEYN | |
| EKFKGKATLTADKSSSTAYMHLSSLTSEDSAVYFCAREEITYAMDYW | |
| GQGTSVTVSS (SEQ ID NO: 22018) | |
| QVQLVQSGAEVKKPGSSVKVSCKASGYTFTSYHIHWVRQAPGQGLE | (SEQ ID NO: |
| WIGWIYPGNVNTEYNEKFKGKATITADESTNTAYMELSSLRSEDTAV | 22021) |
| YYCAREEITYAMDYWGQGTLVTVSSGGGGSGGGGSGGGGSGGDIQ | |
| MTQSPSTLSASVGDRVTITCRSSQSIVHSNGNTYLEWYQQKPGKAPKL | |
| LIYKVSNRFSGVPARFSGSGSGTEFTLTISSLQPDDFATYYCFQGSHVP | |
| RTFGQGTKVEVK (SEQ ID NO: 22020) | |

In some embodiments, the second antigen binding domain comprises an scFv domain that binds to HLA-A*02 antigen. In some embodiments, the scFv domain comprises a sequence of SEQ ID NO: 207, 209, 211, 22018, or 22020, or sequences having at least 90%, at least 95%, at least 97%, at least 99% or is identical thereto. In some embodiments, the scFv domain comprises a sequence of SEQ ID NO: 207, 209 211, 22018, or 22020.

In some embodiments, the engineered receptor comprises a polynucleotide sequence of SEQ ID NO: 21973 or 22008, or sequences having at least 90%, at least 95%, at least 97%, at least 99% or is identical thereto. In some embodiments, the engineered receptor comprises a polynucleotide sequence of SEQ ID NO: 21973 or 22008. In some embodiments, the engineered receptor comprises a polypeptide sequence of SEQ ID NO: 21974 or 22009, or sequences having at least 90%, at least 95%, at least 97%, at least 99% or is identical thereto. In some embodiments, the engineered receptor comprises a polypeptide sequence of SEQ ID NO: 21974 or 22009.

In some embodiments, the engineered receptor comprises a polynucleotide sequence of SEQ ID NO: 21975 or 22010, or sequences having at least 90%, at least 95%, at least 97%, at least 99% or is identical thereto. In some embodiments, the engineered receptor comprises a polynucleotide sequence of SEQ ID NO: 21975 or 22010. In some embodiments, the engineered receptor comprises a polypeptide sequence of SEQ ID NO: 21976 or 22011, or sequences having at least 90%, at least 95%, at least 97%, at least 99% or is identical thereto. In some embodiments, the engineered receptor comprises a polypeptide sequence of SEQ ID NO: 21976 or 22011.

In some embodiments, the extracellular antigen binding domain of the inhibitor receptor comprises an antibody fragment, a single chain Fv antibody fragment (scFv), a β chain variable domain (VB), or a TCR α chain variable domain and a TCR β chain variable domain. In some embodiments, the extracellular antigen binding domain of the inhibitor receptor comprises a heavy chain variable (VH) region and a light chain variable (VL) region. In some embodiments, the VH and VL regions comprise complement determining regions (CDRs) selected from the group of CDRs disclosed in Table 3A.

TABLE 3A
Exemplary HLA-A*02 complementarity determining regions (CDRs)
murine HLA-A*02 Light Chain CDR Sequences

| CDR-L1 | CDR-L2 | CDR-L3 |
|---|---|---|
| RSSQSIVHSNGNTYLE | KVSNRFS (SEQ ID NO: | FQGSHVPRT (SEQ ID |
| (SEQ ID NO: 87) | 21985) | NO: 92) |

murine HLA-A*02 Heavy Chain CDR Sequences

| CDR-H1 | CDR-H2 | CDR-H3 |
|---|---|---|
| SYHIH (SEQ ID NO: | WIYPGNVNTEYNEKFKG | EEITYAMDY (SEQ ID |
| 21986) | (SEQ ID NO: 21987) | NO: 101) |

humanized HLA-A*02 Light Chain CDR Sequences

| CDR-L1 | CDR-L2 | CDR-L3 |
|---|---|---|
| RSSQSIVHSNGNTYLE | KVSNRFS (SEQ ID NO: | FQGSHVPRT (SEQ ID |
| (SEQ ID NO: 87) | 21985) | NO: 92) |

humanized HLA-A*02 Heavy Chain CDR Sequences

| CDR-H1 | CDR-H2 | CDR-H3 |
|---|---|---|
| SYHIH (SEQ ID NO: | WIYPGNVNTEYNEKFKG | EEITYAMDY (SEQ ID |
| 21986) | (SEQ ID NO: 21987) | NO: 101) |

In some embodiments, the HLA-A*02 antigen binding domain comprises a variable heavy (VH) and variable light (VL) region. For example, the HLA-A*02 antigen binding domain is an scFv, antibody or antibody fragment comprising a heavy chain and a light chain, the heavy and light chains comprising a VH and VL region.

In some embodiments, the VH region comprises one or more CDR sequences selected from the group consisting of SYHIH (SEQ ID NO: 21986), WIYPGNVNTEYNEKFKG (SEQ ID NO: 21987), and EEITYAMDY (SEQ ID NO: 101), and the VL region comprises one or more CDR sequences selected from the group consisting of RSSQSIVHSNGNTYLE (SEQ ID NO: 87), KVSNRFS (SEQ ID NO: 21985) and FQGSHVPRT (SEQ ID NO: 92).

In some embodiments, the VH region comprises CDR sequences of SYHIH (SEQ ID NO: 21986), WIYPGNVNTEYNEKFKG (SEQ ID NO: 21987), and EEITYAMDY (SEQ ID NO: 101), and the VL region comprises CDR sequences of RSSQSIVHSNGNTYLE (SEQ ID NO: 87), KVSNRFS (SEQ ID NO: 21985) and FQGSHVPRT (SEQ ID NO: 92).

Illustrative full length HLA-A*02 VH and VL regions are shown in Table 3B below.

TABLE 3B
HLA-A*02 VH and VL sequences

| | |
|---|---|
| murine VL | DVLMTQTPLSLPVSLGDQASISCRSSQSIVHSNGNTYLEWY |
| region (Protein | LQKPGQSPKLLIYKVSNRFSGVPDRFSGSGSGTDFTLKISRV |
| Sequence) | EAEDLGVYYCFQGSHVPRTSGGGTKLEIK (SEQ ID NO: |
| | 21988) |
| murine VL | GATGTTTTGATGACCCAAACTCCACTCTCCCTGCCTGTCA |
| region (DNA | GTCTTGGAGATCAAGCCTCCATCTCTTGCAGATCTAGTCA |
| Sequence) | GAGCATTGTACATAGTAATGGAAACACCTATTTAGAATG |
| | GTACCTGCAGAAACCAGGCCAGTCTCCAAAGCTCCTGAT |
| | CTACAAAGTTTCCAACCGATTTTCTGGGGTCCCAGACAGG |
| | TTCAGTGGCAGTGGATCAGGGACAGATTTCACACTCAAG |
| | ATCAGTAGAGTGGAGGCTGAGGATCTGGGAGTTTATTAC |
| | TGCTTTCAAGGTTCACATGTTCCTCGGACGTCCGGTGGAG |
| | GCACCAAGCTGGAAATCAAA (SEQ ID NO: 21989) |
| murine VH | QVQLQQSGPELVKPGASVRISCKASGYTFTSYHIHWVKQRP |
| region (Protein | GOGLEWIGWIYPGNVNTEYNEKFKGKATLTADKSSSTAY |
| Sequence) | MHLSSLTSEDSAVYFCAREEITYAMDYWGQGTSVTVSS |
| | (SEQ ID NO: 21990) |
| murine VH | CAGGTCCAGCTGCAGCAGTCTGGACCTGAGCTGGTGAAG |
| region (DNA | CCTGGGGCTTCAGTGAGGATATCCTGCAAGGCTTCTGGCT |
| sequence) | ACACCTTCACAAGTTACCATATACATTGGGTGAAGCAGA |
| | GGCCTGGACAGGGACTTGAGTGGATTGGATGGATTTATC |
| | CTGGAAATGTTAATACTGAGTACAATGAGAAGTTCAAGG |
| | GCAAGGCCACACTGACTGCAGACAAATCGTCCAGCACAG |
| | CCTACATGCACCTCAGCAGCCTGACCTCTGAGGACTCTGC |
| | GGTCTATTTCTGTGCCAGAGAGGAGATTACCTATGCTATG |
| | GACTACTGGGGTCAAGGAACCTCAGTCACCGTGTCCTCA |
| | (SEQ ID NO: 21991) |
| humanized VL | DIQMTQSPSTLSASVGDRVTITCRSSQSIVHSNGNTYLEWYQ |
| region (Protein | QKPGKAPKLLIYKVSNRFSGVPARFSGSGSGTEFTLTISSLQP |
| Sequence) | DDFATYYCFQGSHVPRTFGQGTKVEVK (SEQ ID NO: 21997) |
| humanized VL | GACATTCAAATGACCCAGAGCCCATCCACCCTGAGCGCA |
| region (DNA | TCTGTAGGTGACCGGGTCACCATCACTTGTAGATCCAGTC |
| Sequence) | AGAGTATTGTACACAGTAATGGGAACACCTATTTGGAAT |
| | GGTATCAGCAGAAACCAGGTAAAGCCCCAAAATTGCTCA |
| | TCTACAAAGTCTCTAACAGATTTAGTGGTGTACCAGCCAG |
| | GTTCAGCGGTTCCGGAAGTGGTACTGAATTCACCCTCACG |
| | ATCTCCTCTCTCCAGCCAGATGATTTCGCCACTTATTACT |
| | GTTTTCAAGGTTCACATGTGCCGCGCACATTCGGTCAGGG |
| | TACTAAAGTAGAAGTCAAA (SEQ ID NO: 21998) |
| humanized VH | QVQLVQSGAEVKKPGSSVKVSCKASGYTFTSYHIHWVRQA |
| region (Protein | PGQGLEWIGWIYPGNVNTEYNEKFKGKATITADESTNTAY |
| Sequence) | MELSSLRSEDTAVYYCAREEITYAMDYWGQGTLVTVSS |
| | (SEQ ID NO: 21999) |
| humanized VH | CAGGTGCAGCTGGTGCAGTCTGGGGCTGAGGTGAAGAAG |
| region (DNA | CCTGGGTCCTCAGTGAAGGTTTCCTGCAAGGCTTCTGGAT |
| Sequence) | ACACCTTCACTAGCTATCATATACATTGGGTGCGCCAGGC |
| | CCCCGGACAAGGGCTTGAGTGGATCGGATGGATCTACCC |
| | TGGCAATGTTAACACAGAATATAATGAGAAGTTCAAGGG |
| | CAAAGCCACCATTACCGCGGACGAATCCACGAACACAGC |
| | CTACATGGAGCTGAGCAGCCTGAGATCTGAAGACACGGC |
| | TGTGTATTACTGTGCGAGGGAGGAAATTACCTACGCTAT |
| | GGACTACTGGGGCCAGGGAACCCTGGTCACCGTGTCCTC |
| | A (SEQ ID NO: 22000) |

In Table 3B, CDR sequences are bold and underlined.

In some embodiments, the HLA-A*02 antigen binding domain comprises a VL region comprising SEQ ID NO: 21988, or a sequence having at least 90%, at least 95%, at least 97%, at least 98% or at least 99% identity thereto. In some embodiments, the VL region comprises CDR sequences of RSSQSIVHSNGNTYLE (SEQ ID NO: 87), KVSNRFS (SEQ ID NO: 21985) and FQGSHVPRT (SEQ ID NO: 92) and has at least 90%, at least 95%, at least 97%, at least 98% or at least 99% identity to SEQ ID NO: 21988. In some embodiments, the HLA-A*02 antigen binding domain comprises a VL region comprising SEQ ID NO: 21988. In some embodiments, the HLA-A*02 antigen binding domain comprises a VH region comprising SEQ ID NO: 21990, or a sequence having at least 90%, at least 95%, at least 97%, at least 98% or at least 99% identity thereto. In some embodiments, the VH region comprises CDR sequences of SYHIH (SEQ ID NO: 21986), WIYPGNVNTEYNEKFKG (SEQ ID NO: 21987), and EEITYAMDY (SEQ ID NO: 101), and has at least 90%, at least 95%, at least 97%, at least 98% or at least 99% identity to SEQ ID NO: 21990. In some embodiments, the HLA-A*02 antigen binding domain comprises a VH region comprising SEQ ID NO: 21990 and a VL region comprising SEQ ID NO: 21988. In some embodiments, the HLA-A*02 antigen binding domain comprises an antibody fragment, a single chain Fv antibody fragment (scFv), a β chain variable domain (Vβ), or a TCR α chain variable domain and a TCR β chain variable domain. In some embodiments, the HLA-A*02 antigen binding domain comprises an scFv comprising SEQ ID NO: 207, or a sequence having at least 90%, at least 95%, at least 97%, at least 98% or at least 99% identity thereto.

In some embodiments, the HLA-A*02 antigen binding domain comprises a VL region comprising SEQ ID NO: 21997, or a sequence having at least 90%, at least 95%, at least 97%, at least 98% or at least 99% identity thereto. In some embodiments, the VL region comprises CDR sequences of RSSQSIVHSNGNTYLE (SEQ ID NO: 87), KVSNRFS (SEQ ID NO: 21985) and FQGSHVPRT (SEQ ID NO: 92) and has at least 90%, at least 95%, at least 97%, at least 98% or at least 99% identity to SEQ ID NO: 21997. In some embodiments, the HLA-A*02 antigen binding domain comprises a VL region comprising SEQ ID NO: 21997. In some embodiments, the HLA-A*02 antigen binding domain comprises a VH region comprising SEQ ID NO: 21999, or a sequence having at least 90%, at least 95%, at least 97%, at least 98% or at least 99% identity thereto. In some embodiments, the VH region comprises CDR sequences of SYHIH (SEQ ID NO: 21986), WIYPGNVNTEYNEKFKG (SEQ ID NO: 21987), and EEITYAMDY (SEQ ID NO: 101), and has at least 90%, at least 95%, at least 97%, at least 98% or at least 99% identity to SEQ ID NO: 21999. In some embodiments, the HLA-A*02 antigen binding domain comprises a VH region comprising SEQ ID NO: 21999 and a VL region comprising SEQ ID NO: 21997. In some embodiments, the HLA-A*02 antigen binding domain comprises an antibody fragment, a single chain Fv antibody fragment (scFv), a β chain variable domain (Vβ), or a TCR α chain variable domain and a TCR β chain variable domain. In some embodiments, the HLA-A*02 antigen binding domain comprises an scFv comprising SEQ ID NO: 209 or 67, or a sequence having at least 90%, at least 95%, at least 97%, at least 98% or at least 99% identity thereto.

In some embodiments, the non-target antigen comprises HLA-A*01, HLA-A*02, HLA-A*03, HLA-A*11, HLA-B*07 or HLA-C*07. Various scFv domains that bind to or recognize the specified HLA alleles, for use in embodiments described herein, are described in Table 4. Such scFvs include, for example and without limitation, the following mouse and humanized scFv antibodies that bind HLA alleles in a peptide-independent way shown in Table 4 below (complementarity determining regions underlined):

TABLE 4
Additional scFv binding domains
HLA-A*02 antigen binding domains

| | |
|---|---|
| mouse: | scFv (mouse) |
| DVLMTQTPLSLPVSLGDQASISCRSSQSIVHSNGNTYLEWYLQK | DNA Sequence: |
| PGQSPKLLIYKVSNRFSGVPDRFSGSGSGTDFTLKISRVEAEDLG | SEQ ID NO: 75 |
| VYYCFQGSHVPRTSGGGTKLEIKGGGGSGGGGSGGGGSGGQV | |
| QLQQSGPELVKPGASVRISCKASGYTFTSYHIHWVKQRPGQGL | |
| EWIGWIYPGNVNTEYNEKFKGKATLTADKSSSTAYMHLSSLTS | |
| EDSAVYFCAREEITYAMDYWGQGTSVTVSS (SEQ ID NO: 63) | |
| MMTQTPLSLPVSLGDQASISCRSSQSIVHSNGNTYLEWYLQKPG | — |
| QSPKLLIYKVSNRFSGVPDRFSGSGSGTDFTLKISRVEAEDLGVY | |
| YCFQGSHVPRTSGGGTKLEIKGGGGSGGGGSGGGGSGGQVQL | |
| QQSGPELVKPGASVRISCKASGYTFTSYHIHWVKQRPGQGLEW | |
| IGWIYPGNVNTEYNEKFKGKATLTADKSSSTAYMHLSSLTSEDS | |
| AVYFCAREEITYAMDYWGQGTSVTVSSYG (SEQ ID NO: 21943) | |
| humanized: | scFv |
| QVQLVQSGAEVKKPGSSVKVSCKASGYTFTSYHIHWVRQAPG | (humanized) |
| QGLEWMGWIYPGNVNTEYNEKFKGKATITADKSTSTAYMELS | DNA Sequence: |
| SLRSEDTAVYYCAREEITYAMDYWGQGTTVTVSSGGGGSGGG | SEQ ID NO: 76 |
| GSGGGGSGGEIVLTQSPGTLSLSPGERATLSCRSSQSIVHSNGNT | |
| YLEWYQQKPGQAPRLLIYKVSNRFSGIPDRFSGSGSGTDFTLTIS | |
| RLEPEDFAVYYCFQGSHVPRTFGGGTKVEIK (SEQ ID NO: 64) | |
| humanized: | scFv |
| QVQLVQSGAEVKKPGSSVKVSCKASGYTFTSYHIHWVRQAPG | (humanized) |
| QGLEWMGWIYPGNVNTEYNEKFKGKATITADKSTSTAYMELS | DNA Sequence: |
| SLRSEDTAVYYCAREEITYAMDYWGQGTTVTVSSGGGGSGGG | SEQ ID NO: 77 |
| GSGGGGSGGDIVMTQTPLSLPVTPGEPASISCRSSQSIVHSNGNT | |
| YLEWYLQKPGQSPQLLIYKVSNRFSGVPDRFSGSGSGTDFTLKI | |
| SRVEAEDVGVYYCFQGSHVPRTFGGGTKVEIK | |
| (SEQ ID NO: 65) | |
| humanized: | scFv |
| EVQLVESGGGLVKPGGSLRLSCAASGYTFTSYHIHWVRQAPGK | (humanized) |
| GLEWVGWIYPGNVNTEYNEKFKGRFTISRDDSKNTLYLQMNSL | DNA Sequence: |
| KTEDTAVYYCAREEITYAMDYWGQGTTVTVSSGGGGSGGGGS | SEQ ID NO: 78 |
| GGGGSGGDIQMTQSPSSLSASVGDRVTITCRSSQSIVHSNGNTY | |
| LEWYQQKPGKAPKLLIYKVSNRFSGVPSRFSGSGSGTDFTLTISS | |
| LQPEDFATYYCFQGSHVPRTFGGGTKVEIK | |
| (SEQ ID NO: 66) | |
| humanized: | scFv |
| QVQLVQSGAEVKKPGSSVKVSCKASGYTFTSYHIHWVRQAPG | (humanized) |
| QGLEWIGWIYPGNVNTEYNEKFKGKATITADESTNTAYMELSS | DNA Sequence: |
| LRSEDTAVYYCAREEITYAMDYWGQGTLVTVSSGGGGSGGGG | SEQ ID NO: 79 |
| SGGGGSGGDIQMTQSPSTLSASVGDRVTITCRSSQSIVHSNGNT | |
| YLEWYQQKPGKAPKLLIYKVSNRFSGVPARFSGSGSGTEFTLTI | |
| SSLQPDDFATYYCFQGSHVPRTFGQGTKVEVK (SEQ ID NO: 67) | |
| humanized: | |
| QVQLVQSGAEVKKPGSSVKVSCKASGYTFTSYHMHWVRQAPG | scFv |
| QGLEWIGYIYPGNVNTEYNEKFKGKATLTADKSTNTAYMELSS | (humanized) |
| LRSEDTAVYFCAREEITYAMDYWGQGTLVTVSSGGGGSGGGG | DNA Sequence: |
| SGGGGSGGDVQMTQSPSTLSASVGDRVTITCSSSQSIVHSNGNT | SEQ ID NO: 80 |
| YMEWYQQKPGKAPKLLIYKVSNRFSGVPDRFSGSGSGTEFTLTI | |
| SSLQPDDFATYYCHQGSHVPRTFGQGTKVEVK (SEQ ID NO: 68) | |
| mouse: | scFv (mouse) |
| QVQLQQSGPELVKPGASVKMSCKASGYTFTSYHIQWVKQRPG | DNA Sequence: |
| QGLEWIGWIYPGDGSTQYNEKFKGKTTLTADKSSSTAYMLLSS | SEQ ID NO: 81 |
| LTSEDSAIYFCAREGTYYAMDYWGQGTSVTVSSGGGGSGGGG | |
| SGGGGSGGDVLMTQTPLSLPVSLGDQVSISCRSSQSIVHSNGNT | |
| YLEWYLQKPGQSPKLLIYKVSNRFSGVPDRFSGSGSGTDFTLKI | |
| SRVEAEDLGVYYCFQGSHVPRTFGGGTKLEIK (SEQ ID NO: 69) | |
| humanized: | scFv |
| QLQLQESGPGLVKPSETLSLTCTVSGYTFTSYHIQWIRQPPGKG | (humanized) |
| LEWIGWIYPGDGSTQYNEKFKGRATISVDTSKNQFSLNLDSVSA | DNA Sequence: |
| ADTAIYYCAREGTYYAMDYWGKGSTVTVSSGGGGSGGGGSG | SEQ ID NO: 82 |
| GGGSGGDIQMTQSPSSLSASVGDRVTITCRSSQSIVHSNGNTYL | |
| EWYQQKPGKAPKLLIYKVSNRFSGVPSRFSGSGSGTDFTFTISSL | |
| QPEDIATYYCFQGSHVPRTFGPGTKVDIK (SEQ ID NO: 70) | |
| humanized: | scFv |
| EVQLVQSGAELKKPGSSVKVSCKASGYTFTSYHIQWVKQAPGQ | (humanized) |
| GLEWIGWIYPGDGSTQYNEKFKGKATLTVDKSTNTAYMELSSL | DNA Sequence: |
| RSEDTAVYYCAREGTYYAMDYWGQGTLVTVSSGGGGSGGGG | SEQ ID NO: 83 |
| SGGGGSGGDIQMTQSPSTLSASVGDRVTITCRSSQSIVHSNGNT | |
| YLEWYQQKPGKAPKLLIYKVSNRFSGVPSRFSGSGSGTDFTLTI | |
| SSLQPDDFATYYCFQGSHVPRTFGQGTKVEVK (SEQ ID NO: 71) | |
| humanized: | scFv |
| QVQLVQSGAEVKKPGSSVKVSCKASGYTFTSYHIQWVRQAPG | (humanized) |
| QGLEWMGWIYPGDGSTQYNEKFKGRVTITADKSTSTAYMELS | DNA Sequence: |
| SLRSEDTAVYYCAREGTYYAMDYWGQGTTVTVSSGGGGSGG | SEQ ID NO: 84 |
| GGSGGGGSGGEIVLTQSPGTLSLSPGERATLSCRSSQSIVHSNGN | |
| TYLEWYQQKPGQAPRLLIYKVSNRFSGIPDRFSGSGSGTDFTLTI | |
| SRLEPEDFAVYYCFQGSHVPRTFGGGTKVEIK (SEQ ID NO: 72) | |
| humanized: | scFv |
| QVTLKQSGAEVKKPGSSVKVSCTASGYTFTSYHVSWVRQAPG | (humanized) |
| QGLEWLGRIYPGDGSTQYNEKFKGKVTITADKSMDTSFMELTS | DNA Sequence: |
| LTSEDTAVYYCAREGTYYAMDLWGQGTLVTVSSGGGGSGGG | SEQ ID NO: 85 |
| GSGGGGSGGEIVLTQSPGTLSLSPGERATLSCRSSQSIVHSNGNT | |
| YLAWYQQKPGQAPRLLISKVSNRFSGVPDRFSGSGSGTDFTLTI | |
| SRLEPEDFAVYYCQQGSHVPRTFGGGTKVEIK (SEQ ID NO: 73) | |
| humanized: | scFv |
| QVQLVQSGAEVKKPGASVKVSCKASGYTFTSYHMHWVRQAP | (humanized) |
| GQRLEWMGWIYPGDGSTQYNEKFKGKVTITRDTSASTAYMEL | DNA Sequence: |
| SSLRSEDTAVYYCAREGTYYAMDYWGQGTLVTVSSGGGGSGG | SEQ ID NO: 86 |
| GGSGGGGSGGDIVMTQTPLSLPVTPGEPASISCRSSQSIVHSNGN | |
| TYLDWYLQKPGQSPQLLIYKVSNRFSGVPDRFSGSGSGTDFTLK | |
| ISRVEAEDVGVYYCMQGSHVPRTFGGGTKVEIK (SEQ ID NO: | |
| 74) | |
| DVQLQESGPDLVKPSQSLSLTCTVTGYSITSGYSWHWIRQFPRN | BB7.1 scFv |
| KLEWMGYIHFSGSTHYHPSLKSRISITRDTSKNQFFLQLNSVTTE | |
| DTATYYCARGGVVSHYAMDCWGQGTSVTVSSGGGGSGGGGS | |
| GGGGSGGDIQMTQSPASLSVSVGETVTITCRASENIYSNLAWYQ | |
| QKQGKSPHLLVYAATYLPDGVPSRFSGSGSGTQYSLKINSLQSE | |
| DFGSYYCQHFWVTPYTFGGGTKVEIK (SEQ ID NO: 21955) | |

HLA-A*11 antigen binding domains

| | |
|---|---|
| QVQLQESGPGLVKPSQTLSLTCTVSGGSISSGGYYWSWIRQPPG | (SEQ ID NO: |
| KGLEWIGYIYYSGSTYYNPSLKSRVTISVDTSKNQFSLKLSSVTA | 223) |
| ADTAVYYCARHYYYYSMDVWGKGTTVTVSSGGGGSGGGGSG | |
| GGGSGGDIQMTQSPSSLSASVGDRVTITCRASQSISSYLNWYQQ | |
| KPGKAPKLLIYAASSLQSGVPSRFSGSGSGTDFTLTISSLQPEDF | |
| ATYYCQQSYSTPLTFGGGTKVEIK (SEQ ID NO: 52) | |
| QITLKESGPTLVKPTQTLTLTCTFSGFSLSTSGVGVGWIRQPPGK | (SEQ ID NO: |
| ALEWLALIYWNDDKRYSPSLKSRLTITKDTSKNQVVLTMTNM | 224) |
| DPVDTATYYCAHRHMRLSCFDYWGQGTLVTVSSGGGGSGGG | |
| GSGGGGSGGDIQMTQSPSSLSASVGDRVTITCRASQSISSYLNW | |
| YQQKPGKAPKLLIYAASSLQSGVPSRFSGSGSGTDFTLTISSLQP | |
| EDFATYYCQQSYSTPLTFGGGTKVEIK (SEQ ID NO: 53) | |
| QVQLVQSGAEVKKPGASVKVSCKASGYTFTSYAMHWVRQAP | (SEQ ID NO: |
| GQRLEWMGWINAGNGNTKYSQKFQGRVTITRDTSASTAYMEL | 225) |
| SSLRSEDTAVYYCAREGNGANPDAFDIWGQGTMVTVSSGGGG | |
| SGGGGSGGGGSGGDIQMTQSPSSLSASVGDRVTITCRASQSISS | |
| YLNWYQQKPGKAPKLLIYAASSLQSGVPSRFSGSGSGTDFTLTI | |
| SSLQPEDFATYYCQQSYSTPLTFGGGTKVEIK (SEQ ID NO: 54) | |
| EVQLVESGGGLVQPGGSLRLSCAASGFTFSSYDMHWVRQATG | (SEQ ID NO: |
| KGLEWVSAIGTAGDTYYPGSVKGRFTISRENAKNSLYLQMNSL | 226) |
| RAGDTAVYYCARDLPGSYWYFDLWGRGTLVTVSSGGGGSGG | |
| GGSGGGGSGGDIQMTQSPSSLSASVGDRVTITCRASQSISSYLN | |
| WYQQKPGKAPKLLIYAASSLQSGVPSRFSGSGSGTDFTLTISSLQ | |
| PEDFATYYCQQSYSTPLTFGGGTKVEIK (SEQ ID NO: 55) | |
| QVQLQESGPGLVKPSQTLSLTCTVSGGSISSGGYYWSWIRQPPG | (SEQ ID NO: |
| KGLEWIGYIYYSGSTYYNPSLKSRVTISVDTSKNQFSLKLSSVTA | 227) |
| ADTAVYYCARHYYYYYLDVWGKGTTVTVSSGGGGSGGGGSG | |
| GGGSGGDIQMTQSPSSLSASVGDRVTITCRASQSISSYLNWYQQ | |
| KPGKAPKLLIYAASSLQSGVPSRFSGSGSGTDFTLTISSLQPEDF | |
| ATYYCQQSYSTPLTFGGGTKVEIK (SEQ ID NO: 56 | |
| EVQLVESGGGLVQPGGSLRLSCAASGFTFSSYWMHWVRQAPG | (SEQ ID NO: |
| KGLVWVSRINSDGSSTSYADSVKGRFTISRDNAKNTLYLQMNS | 228) |
| LRAEDTAVYYCCLGVLLYNWFDPWGQGTLVTVSSGGGGSGG | |
| GGSGGGGSGGDIQMTQSPSSLSASVGDRVTITCRASQSISSYLN | |
| WYQQKPGKAPKLLIYAASSLQSGVPSRFSGSGSGTDFTLTISSLQ | |
| PEDFATYYCQQSYSTPLTFGGGTKVEIK (SEQ ID NO: 57) | |
| QVQLQESGPGLVKPSQTLSLTCTVSGGSISSGGYYWSWIRQPPG | (SEQ ID NO: |
| KGLEWIGYIYYSGSTYYNPSLKSRVTISVDTSKNQFSLKLSSVTA | 229) |
| ADTAVYYCARHYYYYMDVWGKGTTVTVSSGGGGSGGGGSG | |
| GGGSGGDIQMTQSPSSLSASVGDRVTITCRASQSISSYLNWYQQ | |
| KPGKAPKLLIYAASSLQSGVPSRFSGSGSGTDFTLTISSLQPEDF | |
| ATYYCQQSYSTPLTFGGGTKVEIK (SEQ ID NO: 58) | |
| QITLKESGPTLVKPTQTLTLTCTFSGFSLSTSGVGVGWIRQPPGK | (SEQ ID NO: |
| ALEWLALIYWNDDKRYSPSLKSRLTITKDTSKNQVVLTMTNM | 230) |
| DPVDTATYYCAHKTTSFYFDYWGQGTLVTVSSGGGGSGGGGS | |
| GGGGSGGDIQMTQSPSSLSASVGDRVTITCRASQSISSYLNWYQ | |
| QKPGKAPKLLIYAASSLQSGVPSRFSGSGSGTDFTLTISSLQPED | |
| FATYYCQQSYSTPLTFGGGTKVEIK (SEQ ID NO: 59) | |
| QVQLQESGPGLVKPSQTLSLTCTVSGGSISSGGYYWSWIRQPPG | (SEQ ID NO: |
| KGLEWIGYIYYSGSTYYNPSLKSRVTISVDTSKNQFSLKLSSVTA | 231) |
| ADTAVYYCARHYYYYYMDVWGKGTTVTVSSGGGGSGGGGS | |
| GGGGSGGDIQMTQSPSSLSASVGDRVTITCRASQSISSYLNWYQ | |
| QKPGKAPKLLIYAASSLQSGVPSRFSGSGSGTDFTLTISSLQPED | |
| FATYYCQQSYSTPLTFGGGTKVEIK (SEQ ID NO: 60) | |

HLA-B*07 antigen binding domains

| | |
|---|---|
| QVQLQESGPGLVKPSQTLSLTCTVSGYSITSGYSWHWIRQPPGK | 1.10 scFv |
| GLEWIGYIHFSGSTHYHPSLKSRVTISVDTSKNQFSLKLSSVTAA | |
| DTAVYYCARGGVVSHYAMDCWGQGTTVTVSSGGGGSGGGGS | |
| GGGGSGGDIQMTQSPSSLSASVGDRVTITCRASENIYSNLAWYQ | |
| QKPGKAPKLLIYAATYLPDGVPSRFSGSGSGTDFTLTISSLQPED | |
| FATYYCQHFWVTPYTFGGGTKVEIK (SEQ ID NO: 296) | |
| EVQLVESGGGLVKPGGSLRLSCAASGYSITSGYSWHWVRQAPG | 1.9 scFv |
| KGLEWVSYIHFSGSTHYHPSLKSRFTISRDNAKNSLYLQMNSLR | |
| AEDTAVYYCARGGVVSHYAMDCWGQGTTVTVSSGGGGSGGG | |
| GSGGGGSGGDIQMTQSPSSVSASVGDRVTITCRASENIYSNLAW | |
| YQQKPGKAPKLLIYAATYLPDGVPSRFSGSGSGTDFTLTISSLQP | |
| EDFATYYCQHFWVTPYTFGGGTKVEIK (SEQ ID NO: 297) | |
| EVQLVESGGGLVKPGGSLRLSCAASGYSITSGYSWHWVRQAPG | 1.8 scFv |
| KGLEWVGYIHFSGSTHYHPSLKSRFTISRDDSKNTLYLQMNSLK | |
| TEDTAVYYCARGGVVSHYAMDCWGQGTTVTVSSGGGGSGGG | |
| GSGGGGSGGEIVLTQSPATLSLSPGERATLSCRASENIYSNLAW | |
| YQQKPGQAPRLLIYAATYLPDGIPARFSGSGSGTDFTLTISSLEP | |
| EDFAVYYCQHFWVTPYTFGGGTKVEIK (SEQ ID NO: 298) | |
| QVQLQQSGPGLVKPSQTLSLTCAISGYSITSGYSWHWIRQSPSR | 1.7 scFv |
| GLEWLGYIHFSGSTHYHPSLKSRITINPDTSKNQFSLQLNSVTPE | |
| DTAVYYCARGGVVSHYAMDCWGQGTTVTVSSGGGGSGGGGS | |
| GGGGSGGEIVLTQSPATLSLSPGERATLSCRASENIYSNLAWYQ | |
| QKPGQAPRLLIYAATYLPDGIPARFSGSGSGTDFTLTISRLEPEDF | |
| AVYYCQHFWVTPYTFGGGTKVEIK (SEQ ID NO: 299) | |
| EVQLVESGGGLVKPGGSLRLSCAASGYSITSGYSWHWVRQAPG | 1.6 scFv |
| KGLEWVGYIHFSGSTHYHPSLKSRFTISRDDSKNTLYLQMNSLK | |
| TEDTAVYYCARGGVVSHYAMDCWGQGTTVTVSSGGGGSGGG | |
| GSGGGGSGGDIQMTQSPSSVSASVGDRVTITCRASENIYSNLAW | |
| YQQKPGKAPKLLIYAATYLPDGVPSRFSGSGSGTDFTLTISSLQP | |
| EDFATYYCQHFWVTPYTFGGGTKVEIK (SEQ ID NO: 300) | |
| EVQLVESGGGLVQPGGSLRLSCAASGYSITSGYSWHWVRQAPG | 1.5 scFv |
| KGLEWVSYIHFSGSTHYHPSLKSRFTISRDNSKNTLYLQMNSLR | |
| AEDTAVYYCARGGVVSHYAMDCWGQGTTVTVSSGGGGSGGG | |
| GSGGGGSGGDIQMTQSPSSLSASVGDRVTITCRASENIYSNLAW | |
| YQQKPGKAPKLLIYAATYLPDGVPSRFSGSGSGTDFTLTISSLQP | |
| EDFATYYCQHFWVTPYTFGGGTKVEIK (SEQ ID NO: 301) | |
| EVQLVESGGGLVKPGGSLRLSCAASGYSITSGYSWHWVRQAPG | 1.4 scFv |
| KGLEWVGYIHFSGSTHYHPSLKSRFTISRDDSKNTLYLQMNSLK | |
| TEDTAVYYCARGGVVSHYAMDCWGQGTTVTVSSGGGGSGGG | |
| GSGGGGSGGDIQMTQSPSSLSASVGDRVTITCRASENIYSNLAW | |
| YQQKPGKAPKLLIYAATYLPDGVPSRFSGSGSGTDFTLTISSLQP | |
| EDFATYYCQHFWVTPYTFGGGTKVEIK (SEQ ID NO: 302) | |
| QVQLQQWGAGLLKPSETLSLTCAVYGYSITSGYSWHWIRQPPG | 1.3 scFv |
| KGLEWIGYIHFSGSTHYHPSLKSRVTISVDTSKNQFSLKLSSVTA | |
| ADTAVYYCARGGVVSHYAMDCWGQGTTVTVSSGGGGSGGG | |
| GSGGGGSGGDIQMTQSPSSLSASVGDRVTITCRASENIYSNLAW | |
| YQQKPGKAPKLLIYAATYLPDGVPSRFSGSGSGTDFTLTISSLQP | |
| EDFATYYCQHFWVTPYTFGGGTKVEIK (SEQ ID NO: 303) | |
| QVQLQESGPGLVKPSQTLSLTCTVSGYSITSGYSWHWIRQHPGK | 1.2 scFv |
| GLEWIGYIHFSGSTHYHPSLKSRVTISVDTSKNQFSLKLSSVTAA | |
| DTAVYYCARGGVVSHYAMDCWGQGTTVTVSSGGGGSGGGGS | |
| GGGGSGGDIQMTQSPSSLSASVGDRVTITCRASENIYSNLAWYQ | |
| QKPGKAPKLLIYAATYLPDGVPSRFSGSGSGTDFTLTISSLQPED | |
| FATYYCQHFWVTPYTFGGGTKVEIK (SEQ ID NO: 304) | |
| QVQLQQSGPGLVKPSQTLSLTCAISGYSITSGYSWHWIRQSPSR | 1.1 scFv |
| GLEWLGYIHFSGSTHYHPSLKSRITINPDTSKNQFSLQLNSVTPE | |
| DTAVYYCARGGVVSHYAMDCWGQGTTVTVSSGGGGSGGGGS | |
| GGGGSGGDIQMTQSPSSLSASVGDRVTITCRASENIYSNLAWYQ | |
| QKPGKAPKLLIYAATYLPDGVPSRFSGSGSGTDFTLTISSLQPED | |
| FATYYCQHFWVTPYTFGGGTKVEIK (SEQ ID NO: 305) | |

HLA-C*07 antigen binding domains

| | |
|---|---|
| EVQLLESGGGLVQPGGSLRLSCAASGFTFSSYAMSWVRQAPGKGLE | C7-45 |
| WVSAISGSGGSTYYADSVKGRFTISRDNSKNTLYLQMNSLRAEDTAV | |
| YYCAVSFDWFDPWGQGTLVTVSSGGGGSGGGGSGGGGSGGDIQMT | |
| QSPSSLSASVGDRVTITCRASQSISSYLNWYQQKPGKAPKLLIYAASSL | |
| QSGVPSRFSGSGSGTDFTLTISSLQPEDFATYYCQQSYSTPLTFGGGTK | |
| VEIK (SEQ ID NO: 306) | |
| QVQLQESGPGLVKPSQTLSLTCTVSGGSISSGGYYWSWIRQPPGKGLE | C7-44 |
| WIGYIYYSGSTYYNPSLKSRVTISVDTSKNQFSLKLSSVTAADTAVYY | |
| CARERSISPYYYYYMDVWGKGTTVTVSSGGGGSGGGGSGGGGSGGD | |
| IQMTQSPSSLSASVGDRVTITCRASQSISSYLNWYQQKPGKAPKLLIYA | |
| ASSLQSGVPSRFSGSGSGTDFTLTISSLQPEDFATYYCQQSYSTPLTFG | |
| GGTKVEIK (SEQ ID NO: 307) | |
| QLQLQESGPGLVKPSETLSLTCTVSGGSISSSSYYWGWIRQPPGKGLE | C7-43 |
| WIGSIYYSGSTYYNPSLKSRVTISVDTSKNQFSLKLSSVTAADTAVYY | |
| CARDSVIWYWFDPWGQGTLVTVSSGGGGSGGGGSGGGGSGGQSVL | |
| TQPPSASGTPGQRVTISCSGSSSNIGSNTVNWYQQLPGTAPKLLIYSNN | |
| QRPSGVPDRFSGSKSGTSASLAISGLQSEDEADYYCAAWDDSLNGWV | |
| FGGGTKLTVL (SEQ ID NO: 308) | |
| QVQLQESGPGLVKPSQTLSLTCTVSGGSISSGGYYWSWIRQPPGKGLE | C7-42 |
| WIGYIYYSGSTYYNPSLKSRVTISVDTSKNQFSLKLSSVTAADTAVYY | |
| CAREEILPRLSYYYYMDVWGKGTTVTVSSGGGGSGGGGSGGGGSGG | |
| DIQMTQSPSSLSASVGDRVTITCRASQSISSYLNWYQQKPGKAPKLLIY | |
| AASSLQSGVPSRFSGSGSGTDFTLTISSLQPEDFATYYCQQSYSTPLTF | |
| GGGTKVEIK (SEQ ID NO: 309) | |
| QVQLVQSGSELKKPGASVKVSCKASGYTFTSYAMNWVRQAPGQGLE | C7-41 |
| WMGWINTNTGNPTYAQGFTGRFVFSFDTSVSTAYLQICSLKAEDTAV | |
| YYCARGGRAHSSWYFDLWGRGTLVTVSSGGGGSGGGGSGGGGSGG | |
| DIQMTQSPSSLSASVGDRVTITCRASQSISSYLNWYQQKPGKAPKLLIY | |
| AASSLQSGVPSRFSGSGSGTDFTLTISSLQPEDFATYYCQQSYSTPLTF | |
| GGGTKVEIK (SEQ ID NO: 310) | |
| QVQLQESGPGLVKPSQTLSLTCTVSGGSISSGGYYWSWIRQPPGKGLE | C7-40 |
| WIGYIYYSGSTYYNPSLKSRVTISVDTSKNQFSLKLSSVTAADTAVYY | |
| CARDRIKILPRLGYYYYMDVWGKGTTVTVSSGGGGSGGGGSGGGGS | |
| GGDIQMTQSPSSLSASVGDRVTITCRASQSISSYLNWYQQKPGKAPKL | |
| LIYAASSLQSGVPSRFSGSGSGTDFTLTISSLQPEDFATYYCQQSYSTPL | |
| TFGGGTKVEIK (SEQ ID NO: 311) | |
| QVQLQESGPGLVKPSQTLSLTCTVSGGSISSGGYYWSWIRQPPGKGLE | C7-39 |
| WIGYIYYSGSTYYNPSLKSRVTISVDTSKNQFSLKLSSVTAADTAVYY | |
| CARDTVIHYYYYMDVWGKGTTVTVSSGGGGSGGGGSGGGGSGGDI | |
| QMTQSPSSLSASVGDRVTITCRASQSISSYLNWYQQKPGKAPKLLIYA | |
| ASSLQSGVPSRFSGSGSGTDFTLTISSLQPEDFATYYCQQSYSTPLTFG | |
| GGTKVEIK (SEQ ID NO: 312) | |
| QVQLQESGPGLVKPSQTLSLTCTVSGGSISSGGYYWSWIRQPPGKGLE | C7-38 |
| WIGYIYYSGSTYYNPSLKSRVTISVDTSKNQFSLKLSSVTAADTAVYY | |
| CARDVIVEVFLSYYYYMDVWGKGTTVTVSSGGGGSGGGGSGGGGS | |
| GGDIQMTQSPSSLSASVGDRVTITCRASQSISSYLNWYQQKPGKAPKL | |
| LIYAASSLQSGVPSRFSGSGSGTDFTLTISSLQPEDFATYYCQQSYSTPL | |
| TFGGGTKVEIK (SEQ ID NO: 313) | |
| QVQLQESGPGLVKPSQTLSLTCTVSGGSISSGGYYWSWIRQPPGKGLE | C7-37 |
| WIGYIYYSGSTYYNPSLKSRVTISVDTSKNQFSLKLSSVTAADTAVYY | |
| CARDIFIHYYYYMDVWGKGTTVTVSSGGGGSGGGGSGGGGSGGDIQ | |
| MTQSPSSLSASVGDRVTITCRASQSISSYLNWYQQKPGKAPKLLIYAA | |
| SSLQSGVPSRFSGSGSGTDFTLTISSLQPEDFATYYCQQSYSTPLTFGG | |
| GTKVEIK (SEQ ID NO: 314) | |
| EVQLVESGGGLVQPGGSLRLSCAASGFTFSSYSMNWVRQAPGKGLE | C7-36 |
| WVSYISSSSSTIYYADSVKGRFTISRDNAKNSLYLQMNSLRAEDTAVY | |
| YCARDGTFYSYSPYYFDYWGQGTLVTVSSGGGGSGGGGSGGGGSGG | |
| DIQMTQSPSSLSASVGDRVTITCRASQSISSYLNWYQQKPGKAPKLLIY | |
| AASSLQSGVPSRFSGSGSGTDFTLTISSLQPEDFATYYCQQSYSTPLTF | |
| GGGTKVEIK (SEQ ID NO: 315) | |
| QVQLQESGPGLVKPSQTLSLTCTVSGGSISSGGYYWSWIRQPPGKGLE | C7-35 |
| WIGYIYYSGSTYYNPSLKSRVTISVDTSKNQFSLKLSSVTAADTAVYY | |
| CAREWIKILPRLGYYYYMDVWGKGTTVTVSSGGGGSGGGGSGGGGS | |
| GGDIQMTQSPSSLSASVGDRVTITCRASQSISSYLNWYQQKPGKAPKL | |
| LIYAASSLQSGVPSRFSGSGSGTDFTLTISSLQPEDFATYYCQQSYSTPL | |
| TFGGGTKVEIK (SEQ ID NO: 316) | |
| QVQLQESGPGLVKPSQTLSLTCTVSGGSISSGGYYWSWIRQPPGKGLE | C7-34 |
| WIGYIYYSGSTYYNPSLKSRVTISVDTSKNQFSLKLSSVTAADTAVYY | |
| CARDRSLYYYYYMDVWGKGTTVTVSSGGGGSGGGGSGGGGSGGDI | |
| QMTQSPSSLSASVGDRVTITCRASQSISSYLNWYQQKPGKAPKLLIYA | |
| ASSLQSGVPSRFSGSGSGTDFTLTISSLQPEDFATYYCQQSYSTPLTFG | |
| GGTKVEIK (SEQ ID NO: 317) | |
| QVQLQESGPGLVKPSQTLSLTCTVSGGSISSGGYYWSWIRQPPGKGLE | C7-33 |
| WIGYIYYSGSTYYNPSLKSRVTISVDTSKNQFSLKLSSVTAADTAVYY | |
| CARDKILAPNYYYYMDVWGKGTTVTVSSGGGGSGGGGSGGGGSGG | |
| DIQMTQSPSSLSASVGDRVTITCRASQSISSYLNWYQQKPGKAPKLLIY | |
| AASSLQSGVPSRFSGSGSGTDFTLTISSLQPEDFATYYCQQSYSTPLTF | |
| GGGTKVEIK (SEQ ID NO: 318) | |
| QVQLQESGPGLVKPSQTLSLTCTVSGGSISSGGYYWSWIRQPPGKGLE | C7-32 |
| WIGYIYYSGSTYYNPSLKSRVTISVDTSKNQFSLKLSSVTAADTAVYY | |
| CAREKSWKYFYYYYYYMDVWGKGTTVTVSSGGGGSGGGGSGGGG | |
| SGGDIQMTQSPSSLSASVGDRVTITCRASQSISSYLNWYQQKPGKAPK | |
| LLIYAASSLQSGVPSRFSGSGSGTDFTLTISSLQPEDFATYYCQQSYSTP | |
| LTFGGGTKVEIK (SEQ ID NO: 319) | |
| QVQLQESGPGLVKPSQTLSLTCTVSGGSISSGGYYWSWIRQPPGKGLE | C7-31 |
| WIGYIYYSGSTYYNPSLKSRVTISVDTSKNQFSLKLSSVTAADTAVYY | |
| CARENTSTIPYYYYYMDVWGKGTTVTVSSGGGGSGGGGSGGGGSGG | |
| DIQMTQSPSSLSASVGDRVTITCRASQSISSYLNWYQQKPGKAPKLLIY | |
| AASSLQSGVPSRFSGSGSGTDFTLTISSLQPEDFATYYCQQSYSTPLTF | |
| GGGTKVEIK (SEQ ID NO: 320) | |
| QVQLQESGPGLVKPSQTLSLTCTVSGGSISSGGYYWSWIRQPPGKGLE | C7-30 |
| WIGYIYYSGSTYYNPSLKSRVTISVDTSKNQFSLKLSSVTAADTAVYY | |
| CAREDVDKNTSTIYYYYYYMDVWGKGTTVTVSSGGGGSGGGGSGG | |
| GGSGGDIQMTQSPSSLSASVGDRVTITCRASQSISSYLNWYQQKPGKA | |
| PKLLIYAASSLQSGVPSRFSGSGSGTDFTLTISSLQPEDFATYYCQQSYS | |
| TPLTFGGGTKVEIK (SEQ ID NO: 321) | |
| QVQLVESGGGLVKPGGSLRLSCAASGFTFSDYYMSWIRQAPGKGLE | C7-29 |
| WVSYISSSGSTIYYADSVKGRFTISRDNAKNSLYLQMNSLRAEDTAVY | |
| YCARDGGDIVSSSAIYWYFDLWGRGTLVTVSSGGGGSGGGGSGGGG | |
| SGGAIQLTQSPSSLSASVGDRVTITCRASQGISSALAWYQQKPGKAPK | |
| LLIYDASSLESGVPSRFSGSGSGTDFTLTISSLQPEDFATYYCQQFNSYP | |
| LTFGGGTKVEIK (SEQ ID NO: 322) | |
| QVQLQESGPGLVKPSQTLSLTCTVSGGSISSGGYYWSWIRQPPGKGLE | C7-28 |
| WIGYIYYSGSTYYNPSLKSRVTISVDTSKNQFSLKLSSVTAADTAVYY | |
| CARDLILPPYYYYYMDVWGKGTTVTVSSGGGGSGGGGSGGGGSGG | |
| DIQMTQSPSSLSASVGDRVTITCRASQSISSYLNWYQQKPGKAPKLLIY | |
| AASSLQSGVPSRFSGSGSGTDFTLTISSLQPEDFATYYCQQSYSTPLTF | |
| GGGTKVEIK (SEQ ID NO: 323) | |
| QVQLQESGPGLVKPSQTLSLTCTVSGGSISSGGYYWSWIRQPPGKGLE | C7-27 |
| WIGYIYYSGSTYYNPSLKSRVTISVDTSKNQFSLKLSSVTAADTAVYY | |
| CARETWIKILPRYYYYYYYMDVWGKGTTVTVSSGGGGSGGGGSGG | |
| GGSGGDIQMTQSPSSLSASVGDRVTITCRASQSISSYLNWYQQKPGKA | |
| PKLLIYAASSLQSGVPSRFSGSGSGTDFTLTISSLQPEDFATYYCQQSYS | |
| TPLTFGGGTKVEIK (SEQ ID NO: 324) | |
| QVQLQESGPGLVKPSQTLSLTCTVSGGSISSGGYYWSWIRQPPGKGLE | C7-26 |
| WIGYIYYSGSTYYNPSLKSRVTISVDTSKNQFSLKLSSVTAADTAVYY | |
| CARDLSRYYYYYMDVWGKGTTVTVSSGGGGSGGGGSGGGGSGGDI | |
| QMTQSPSSLSASVGDRVTITCRASQSISSYLNWYQQKPGKAPKLLIYA | |
| ASSLQSGVPSRFSGSGSGTDFTLTISSLQPEDFATYYCQQSYSTPLTFG | |
| GGTKVEIK (SEQ ID NO: 325) | |
| EVQLVESGGGLVQPGGSLRLSCAASGFTFSSYSMNWVRQAPGKGLE | C7-25 |
| WVSYISSSSSTIYYADSVKGRFTISRDNAKNSLYLQMNSLRAEDTAVY | |
| YCAREHIVLCFDYWGQGTLVTVSSGGGGSGGGGSGGGGSGGDIQMT | |
| QSPSSLSASVGDRVTITCRASQGISSWLAWYQQKPEKAPKSLIYAASS | |
| LQSGVPSRFSGSGSGTDFTLTISSLQPEDFATYYCQQYNSYPLTFGGGT | |
| KVEIK (SEQ ID NO: 326) | |
| QVQLQESGPGLVKPSQTLSLTCTVSGGSISSGGYYWSWIRQPPGKGLE | C7-24 |
| WIGYIYYSGSTYYNPSLKSRVTISVDTSKNQFSLKLSSVTAADTAVYY | |
| CARDKILPRPYYYYYMDVWGKGTTVTVSSGGGGSGGGGSGGGGSG | |
| GDIQMTQSPSSLSASVGDRVTITCRASQSISSYLNWYQQKPGKAPKLLI | |
| YAASSLQSGVPSRFSGSGSGTDFTLTISSLQPEDFATYYCQQSYSTPLT | |
| FGGGTKVEIK (SEQ ID NO: 327) | |
| QVQLVQSGAEVKKPGASVKVSCKASGYTFTSYGISWVRQAPGQGLE | C7-23 |
| WMGWISAYNGNTNYAQKLQGRVTMTTDTSTSTAYMELRSLRSDDT | |
| AVYYCARGSNEYFQHWGQGTLVTVSSGGGGSGGGGSGGGGSGGQS | |
| ALTQPPSASGSPGQSVTISCTGTSSDVGGYNYVSWYQQHPGKAPKLM | |
| IYEVSKRPSGVPDRFSGSKSGNTASLTVSGLQAEDEADYYCSSYAGSN | |
| NWVFGGGTKLTVL (SEQ ID NO: 328) | |
| QVQLVQSGSELKKPGASVKVSCKASGYTFTSYAMNWVRQAPGQGLE | C7-22 |
| WMGWINTNTGNPTYAQGFTGRFVFSFDTSVSTAYLQICSLKAEDTAV | |
| YYCARGTSYWYFDLWGRGTLVTVSSGGGGSGGGGSGGGGSGGDIQ | |
| MTQSPSSLSASVGDRVTITCRASQSISSYLNWYQQKPGKAPKLLIYAA | |
| SSLQSGVPSRFSGSGSGTDFTLTISSLQPEDFATYYCQQSYSTPLTFGG | |
| GTKVEIK (SEQ ID NO: 329) | |
| QVQLQESGPGLVKPSQTLSLTCTVSGGSISSGGYYWSWIRQPPGKGLE | C7-21 |
| WIGYIYYSGSTYYNPSLKSRVTISVDTSKNQFSLKLSSVTAADTAVYY | |
| CAREEIVEVFYYYYMDVWGKGTTVTVSSGGGGSGGGGSGGGGSGG | |
| DIQMTQSPSSLSASVGDRVTITCRASQSISSYLNWYQQKPGKAPKLLIY | |
| AASSLQSGVPSRFSGSGSGTDFTLTISSLQPEDFATYYCQQSYSTPLTF | |
| GGGTKVEIK (SEQ ID NO: 330) | |
| EVQLLESGGGLVQPGGSLRLSCAASGFTFSSYAMSWVRQAPGKGLE | C7-20 |
| WVSAISGSGGSTYYADSVKGRFTISRDNSKNTLYLQMNSLRAEDTAV | |
| YYCAKVDDYYFDYWGQGTLVTVSSGGGGSGGGGSGGGGSGGDIQM | |
| TQSPSSLSASVGDRVTITCRASQSISSYLNWYQQKPGKAPKLLIYAASS | |
| LQSGVPSRFSGSGSGTDFTLTISSLQPEDFATYYCQQSYSTPLTFGGGT | |
| KVEIK (SEQ ID NO: 331) | |
| EVQLVESGGGLVQPGGSLRLSCAASGFTFSSYWMHWVRQAPGKGLV | C7-19 |
| WVSRINSDGSSTSYADSVKGRFTISRDNAKNTLYLQMNSLRAEDTAV | |
| YYCAWSTNILLSYTKAFDIWGQGTMVTVSSGGGGSGGGGSGGGGSG | |
| GDIQMTQSPSSLSASVGDRVTITCRASQSISSYLNWYQQKPGKAPKLLI | |
| YAASSLQSGVPSRFSGSGSGTDFTLTISSLQPEDFATYYCQQSYSTPLT | |
| FGGGTKVEIK (SEQ ID NO: 332) | |
| QVQLQESGPGLVKPSQTLSLTCTVSGGSISSGGYYWSWIRQPPGKGLE | C7-18 |
| WIGYIYYSGSTYYNPSLKSRVTISVDTSKNQFSLKLSSVTAADTAVYY | |
| CARDKTYYYYYYMDVWGKGTTVTVSSGGGGSGGGGSGGGGSGGDI | |
| QMTQSPSSLSASVGDRVTITCRASQSISSYLNWYQQKPGKAPKLLIYA | |
| ASSLQSGVPSRFSGSGSGTDFTLTISSLQPEDFATYYCQQSYSTPLTFG | |
| GGTKVEIK (SEQ ID NO: 333) | |
| QVQLQESGPGLVKPSQTLSLTCTVSGGSISSGGYYWSWIRQPPGKGLE | C7-17 |
| WIGYIYYSGSTYYNPSLKSRVTISVDTSKNQFSLKLSSVTAADTAVYY | |
| CAREKYFHDKYFHDYYYYYMDVWGKGTTVTVSSGGGGSGGGGSG | |
| GGGSGGDIQMTQSPSSLSASVGDRVTITCRASQSISSYLNWYQQKPGK | |
| APKLLIYAASSLQSGVPSRFSGSGSGTDFTLTISSLQPEDFATYYCQQS | |
| YSTPLTFGGGTKVEIK (SEQ ID NO: 334) | |
| QVQLQESGPGLVKPSQTLSLTCTVSGGSISSGGYYWSWIRQPPGKGLE | C7-16 |
| WIGYIYYSGSTYYNPSLKSRVTISVDTSKNQFSLKLSSVTAADTAVYY | |
| CARDTSVYYYYYMDVWGKGTTVTVSSGGGGSGGGGSGGGGSGGDI | |
| QMTQSPSSLSASVGDRVTITCRASQSISSYLNWYQQKPGKAPKLLIYA | |
| ASSLQSGVPSRFSGSGSGTDFTLTISSLQPEDFATYYCQQSYSTPLTFG | |
| GGTKVEIK (SEQ ID NO: 335) | |
| QVQLQESGPGLVKPSQTLSLTCTVSGGSISSGGYYWSWIRQPPGKGLE | C7-15 |
| WIGYIYYSGSTYYNPSLKSRVTISVDTSKNQFSLKLSSVTAADTAVYY | |
| CAREKILPYYYYYYMDVWGKGTTVTVSSGGGGSGGGGSGGGGSGG | |
| DIQMTQSPSSLSASVGDRVTITCRASQSISSYLNWYQQKPGKAPKLLIY | |
| AASSLQSGVPSRFSGSGSGTDFTLTISSLQPEDFATYYCQQSYSTPLTF | |
| GGGTKVEIK (SEQ ID NO: 336) | |
| EVQLVESGGGLVQPGGSLRLSCAASGFTFSSYSMNWVRQAPGKGLE | C7-14 |
| WVSYISSSSSTIYYADSVKGRFTISRDNAKNSLYLQMNSLRAEDTAVY | |
| YCAIQWIYIYINPRGFIFLHDAFDIWGQGTMVTVSSGGGGSGGGGSGG | |
| GGSGGQSVLTQPPSASGTPGQRVTISCSGSSSNIGSNTVNWYQQLPGT | |
| APKLLIYSNNQRPSGVPDRFSGSKSGTSASLAISGLQSEDEADYYCAA | |
| WDDSLNGWVFGGGTKLTVL (SEQ ID NO: 337) | |
| QVQLQQSGPGLVKPSQTLSLTCAISGDSVSSNSAAWNWIRQSPSRGLE | C7-13 |
| WLGRTYYRSKWYNDYAVSVKSRITINPDTSKNQFSLQLNSVTPEDTA | |
| VYYCAKEDVDFHHDAFDIWGQGTMVTVSSGGGGSGGGGSGGGGSG | |
| GDIQMTQSPSSLSASVGDRVTITCRASQSISSYLNWYQQKPGKAPKLLI | |
| YAASSLQSGVPSRFSGSGSGTDFTLTISSLQPEDFATYYCQQSYSTPLT | |
| FGGGTKVEIK (SEQ ID NO: 338) | |
| QVQLQESGPGLVKPSQTLSLTCTVSGGSISSGGYYWSWIRQPPGKGLE | C7-12 |
| WIGYIYYSGSTYYNPSLKSRVTISVDTSKNQFSLKLSSVTAADTAVYY | |
| CAREGVDKNTSTIYYYYYYMDVWGKGTTVTVSSGGGGSGGGGSGG | |
| GGSGGDIQMTQSPSSLSASVGDRVTITCRASQSISSYLNWYQQKPGKA | |
| PKLLIYAASSLQSGVPSRFSGSGSGTDFTLTISSLQPEDFATYYCQQSYS | |
| TPLTFGGGTKVEIK (SEQ ID NO: 339) | |
| EVQLVESGGGLVQPGGSLRLSCAASGFTFSSYSMNWVRQAPGKGLE | C7-11 |
| WVSYISSSSSTIYYADSVKGRFTISRDNAKNSLYLQMNSLRAEDTAVY | |
| YCARDRRGYFDLWGRGTLVTVSSGGGGSGGGGSGGGGSGGDIQMT | |
| QSPSSLSASVGDRVTITCRASQGISSWLAWYQQKPEKAPKSLIYAASS | |
| LQSGVPSRFSGSGSGTDFTLTISSLQPEDFATYYCQQYNSYPLTFGGGT | |
| KVEIK (SEQ ID NO: 340) | |
| EVQLVQSGAEVKKPGATVKISCKVSGYTFTDYYMHWVQQAPGKGLE | C7-10 |
| WMGLVDPEDGETIYAEKFQGRVTITADTSTDTAYMELSSLRSEDTAV | |
| YYCATGIHVDIRSMEDWFDPWGQGTLVTVSSGGGGSGGGGSGGGGS | |
| GGDIQMTQSPSSLSASVGDRVTITCRASQSISSYLNWYQQKPGKAPKL | |
| LIYAASSLQSGVPSRFSGSGSGTDFTLTISSLQPEDFATYYCQQSYSTPL | |
| TFGGGTKVEIK (SEQ ID NO: 341) | |
| QVQLQESGPGLVKPSQTLSLTCTVSGGSISSGGYYWSWIRQPPGKGLE | C7-9 |
| WIGYIYYSGSTYYNPSLKSRVTISVDTSKNQFSLKLSSVTAADTAVYY | |
| CARDIGTSYYYYMDVWGKGTTVTVSSGGGGSGGGGSGGGGSGGDIQ | |
| MTQSPSSLSASVGDRVTITCRASQSISSYLNWYQQKPGKAPKLLIYAA | |
| SSLQSGVPSRFSGSGSGTDFTLTISSLQPEDFATYYCQQSYSTPLTFGG | |
| GTKVEIK (SEQ ID NO: 342) | |
| QVQLQESGPGLVKPSQTLSLTCTVSGGSISSGGYYWSWIRQPPGKGLE | C7-8 |
| WIGYIYYSGSTYYNPSLKSRVTISVDTSKNQFSLKLSSVTAADTAVYY | |
| CAREVVEVFLYYYYYMDVWGKGTTVTVSSGGGGSGGGGSGGGGSG | |
| GDIQMTQSPSSLSASVGDRVTITCRASQSISSYLNWYQQKPGKAPKLLI | |
| YAASSLQSGVPSRFSGSGSGTDFTLTISSLQPEDFATYYCQQSYSTPLT | |
| FGGGTKVEIK (SEQ ID NO: 343) | |
| QVQLQESGPGLVKPSQTLSLTCTVSGGSISSGGYYWSWIRQPPGKGLE | C7-7 |
| WIGYIYYSGSTYYNPSLKSRVTISVDTSKNQFSLKLSSVTAADTAVYY | |
| CARDLYYYYYYYMDVWGKGTTVTVSSGGGGSGGGGSGGGGSGGDI | |
| QMTQSPSSLSASVGDRVTITCRASQSISSYLNWYQQKPGKAPKLLIYA | |
| ASSLQSGVPSRFSGSGSGTDFTLTISSLQPEDFATYYCQQSYSTPLTFG | |
| GGTKVEIK (SEQ ID NO: 344) | |
| QVQLQESGPGLVKPSQTLSLTCTVSGGSISSGGYYWSWIRQPPGKGLE | C7-6 |
| WIGYIYYSGSTYYNPSLKSRVTISVDTSKNQFSLKLSSVTAADTAVYY | |
| CARESWKYFYPRGSIFIHYYYYMDVWGKGTTVTVSSGGGGSGGGGS | |
| GGGGSGGDIQMTQSPSSLSASVGDRVTITCRASQSISSYLNWYQQKPG | |
| KAPKLLIYAASSLQSGVPSRFSGSGSGTDFTLTISSLQPEDFATYYCQQ | |
| SYSTPLTFGGGTKVEIK (SEQ ID NO: 345) | |
| QVQLQESGPGLVKPSQTLSLTCTVSGGSISSGGYYWSWIRQPPGKGLE | C7-5 |
| WIGYIYYSGSTYYNPSLKSRVTISVDTSKNQFSLKLSSVTAADTAVYY | |
| CARDRIVEVFYYYYMDVWGKGTTVTVSSGGGGSGGGGSGGGGSGG | |
| DIQMTQSPSSLSASVGDRVTITCRASQSISSYLNWYQQKPGKAPKLLIY | |
| AASSLQSGVPSRFSGSGSGTDFTLTISSLQPEDFATYYCQQSYSTPLTF | |
| GGGTKVEIK (SEQ ID NO: 346) | |
| QVQLQESGPGLVKPSQTLSLTCTVSGGSISSGGYYWSWIRQPPGKGLE | C7-4 |
| WIGYIYYSGSTYYNPSLKSRVTISVDTSKNQFSLKLSSVTAADTAVYY | |
| CAREKYFHDWLYYYYYMDVWGKGTTVTVSSGGGGSGGGGSGGGG | |
| SGGDIQMTQSPSSLSASVGDRVTITCRASQSISSYLNWYQQKPGKAPK | |
| LLIYAASSLQSGVPSRFSGSGSGTDFTLTISSLQPEDFATYYCQQSYSTP | |
| LTFGGGTKVEIK (SEQ ID NO: 347) | |
| QVQLQESGPGLVKPSQTLSLTCTVSGGSISSGGYYWSWIRQPPGKGLE | C7-3 |
| WIGYIYYSGSTYYNPSLKSRVTISVDTSKNQFSLKLSSVTAADTAVYY | |
| CARDLVDKNTSYYYYYMDVWGKGTTVTVSSGGGGSGGGGSGGGGS | |
| GGDIQMTQSPSSLSASVGDRVTITCRASQSISSYLNWYQQKPGKAPKL | |
| LIYAASSLQSGVPSRFSGSGSGTDFTLTISSLQPEDFATYYCQQSYSTPL | |
| TFGGGTKVEIK (SEQ ID NO: 348) | |
| QVQLVQSGAEVKKPGASVKVSCKASGYTFTSYGISWVRQAPGQGLE | C7-2 |
| WMGWISAYNGNTNYAQKLQGRVTMTTDTSTSTAYMELRSLRSDDT | |
| AVYYCARVQNEYFQHWGQGTLVTVSSGGGGSGGGGSGGGGSGGQS | |
| ALTQPPSASGSPGQSVTISCTGTSSDVGGYNYVSWYQQHPGKAPKLM | |
| IYEVSKRPSGVPDRFSGSKSGNTASLTVSGLQAEDEADYYCSSYAGSN | |
| NWVFGGGTKLTVL (SEQ ID NO: 349) | |
| QVQLVESGGGLVKPGGSLRLSCAASGFTFSDYYMSWIRQAPGKGLE | C7-1 |
| WVSYISSSGSTIYYADSVKGRFTISRDNAKNSLYLQMNSLRAEDTAVY | |
| YCATANWFDPWGQGTLVTVSSGGGGSGGGGSGGGGSGGDIQMTQS | |
| PSSVSASVGDRVTITCRASQGISSWLAWYQQKPGKAPKLLIYAASSLQ | |
| SGVPSRFSGSGSGTDFTLTISSLQPEDFATYYCQQANSFPLTFGGGTKV | |
| EIK (SEQ ID NO: 350) | |

HLA-A*03 antigen binding domain sequences

| | |
|---|---|
| QVQLVQSGAEVKKPGASVKVSCKASGYTFTSYGISWVRQAPG | 15 |
| QGLEWMGWISAYNGNTNYAQKLQGRVTMTTDTSTSTAYMEL | |
| RSLRSDDTAVYYCARERVSQRGAFDIWGQGTMVTVSSGGGGS | |
| GGGGSGGGGSGGDIQMTQSPSSLSASVGDRVTITCRASQSISSY | |
| LNWYQQKPGKAPKLLIYAASSLQSGVPSRFSGSGSGTDFTLTISS | |
| LQPEDFATYYCQQSYSTPLTFGGGTKVEIK (SEQ ID NO: 351) | |
| EVQLVESGGGLVQPGGSLRLSCAASGFTFSSYSMNWVRQAPGK | 16 |
| GLEWVSYISSSSSTIYYADSVKGRFTISRDNAKNSLYLQMNSLR | |
| AEDTAVYYCARGNPDKDPFDYWGQGTLVTVSSGGGGSGGGG | |
| SGGGGSGGDIQMTQSPSSLSASVGDRVTITCRASQSISSYLNWY | |
| QQKPGKAPKLLIYAASSLQSGVPSRFSGSGSGTDFTLTISSLQPE | |
| DFATYYCQQSYSTPLTFGGGTKVEIK (SEQ ID NO: 352) | |
| QVQLQESGPGLVKPSETLSLTCTVSGGSVSSGSYYWSWIRQPPG | 17 |
| KGLEWIGYIYYSGSTNYNPSLKSRVTISVDTSKNQFSLKLSSVTA | |
| ADTAVYYCARDFYCTNWYFDLWGRGTLVTVSSGGGGSGGGG | |
| SGGGGSGGDIQMTQSPSSLSASVGDRVTITCRASQSISSYLNWY | |
| QQKPGKAPKLLIYAASSLQSGVPSRFSGSGSGTDFTLTISSLQPE | |
| DFATYYCQQSYSTPLTFGGGTKVEIK (SEQ ID NO: 353) | |
| QVQLQESGPGLVKPSETLSLTCTVSGGSISSYYWSWIRQPPGKG | 18 |
| LEWIGYIYYSGSTNYNPSLKSRVTISVDTSKNQFSLKLSSVTAAD | |
| TAVYYCARESSSGSYWYFDLWGRGTLVTVSSGGGGSGGGGSG | |
| GGGSGGDIQMTQSPSSLSASVGDRVTITCRASQSISSYLNWYQQ | |
| KPGKAPKLLIYAASSLQSGVPSRFSGSGSGTDFTLTISSLQPEDF | |
| ATYYCQQSYSTPLTFGGGTKVEIK (SEQ ID NO: 354) | |
| EVQLVQSGAEVKKPGESLKISCKGSGYSFTSYWIGWVRQMPGK | 19 |
| GLEWMGIIYPGDSDTRYSPSFQGQVTISADKSISTAYLQWSSLK | |
| ASDTAMYYCARDSGYKYNLYYYYYYMDVWGKGTTVTVSSG | |
| GGGSGGGGSGGGGSGGDIQMTQSPSSLSASVGDRVTITCRASQ | |
| SISSYLNWYQQKPGKAPKLLIYAASSLQSGVPSRFSGSGSGTDF | |
| TLTISSLQPEDFATYYCQQSYSTPLTFGGGTKVEIK (SEQ ID NO: | |
| 355) | |
| QVQLVQSGAEVKKPGASVKVSCKASGYTFTSYGISWVRQAPG | 20 |
| QGLEWMGWISAYNGNTNYAQKLQGRVTMTTDTSTSTAYMEL | |
| RSLRSDDTAVYYCARGGDLSHYYYYMDVWGKGTTVTVSSGG | |
| GGSGGGGSGGGGSGGQTVVTQEPSLTVSPGGTVTLTCASSTGA | |
| VTSGYYPNWFQQKPGQAPRALIYSTSNKHSWTPARFSGSLLGG | |
| KAALTLSGVQPEDEAEYYCLLYYGGAQWVFGGGTKLTVL | |
| (SEQ ID NO: 356) | |
| QVQLVQSGAEVKKPGASVKVSCKASGYTFTSYGISWVRQAPG | 21 |
| QGLEWMGWISAYNGNTNYAQKLQGRVTMTTDTSTSTAYMEL | |
| RSLRSDDTAVYYCARENRRYNSCYYFDYWGQGTLVTVSSGGG | |
| GSGGGGSGGGGSGGDIQMTQSPSSLSASVGDRVTITCRASQSIS | |
| SYLNWYQQKPGKAPKLLIYAASSLQSGVPSRFSGSGSGTDFTLT | |
| ISSLQPEDFATYYCQQSYSTPLTFGGGTKVEIK (SEQ ID NO: 357) | |
| QVQLVQSGAEVKKPGASVKVSCKASGYTFTSYGISWVRQAPG | 22 |
| QGLEWMGWISAYNGNTNYAQKLQGRVTMTTDTSTSTAYMEL | |
| RSLRSDDTAVYYCARGGDLSHYYYYLDVWGKGTTVTVSSGG | |
| GGSGGGGSGGGGSGGQTVVTQEPSLTVSPGGTVTLTCASSTGA | |
| VTSGYYPNWFQQKPGQAPRALIYSTSNKHSWTPARFSGSLLGG | |
| KAALTLSGVQPEDEAEYYCLLYYGGAQWVFGGGTKLTVL | |
| (SEQ ID NO: 358) | |
| EVQLVESGGGLVQPGGSLRLSCAASGFTVSSNYMSWVRQAPG | 23 |
| KGLEWVSVIYSGGSTYYADSVKGRFTISRDNSKNTLYLQMNSL | |
| RAEDTAVYYCARATLLSLSYDAFDIWGQGTMVTVSSGGGGSG | |
| GGGSGGGGSGGDIQMTQSPSSLSASVGDRVTITCRASQSISSYL | |
| NWYQQKPGKAPKLLIYAASSLQSGVPSRFSGSGSGTDFTLTISSL | |
| QPEDFATYYCQQSYSTPLTFGGGTKVEIK (SEQ ID NO: 359) | |
| QVQLVQSGAEVKKPGASVKVSCKASGYTFTSYGISWVRQAPG | 24 |
| QGLEWMGWISAYNGNTNYAQKLQGRVTMTTDTSTSTAYMEL | |
| RSLRSDDTAVYYCARGGDLSHYYYMDVWGKGTTVTVSSGGG | |
| GSGGGGSGGGGSGGQTVVTQEPSLTVSPGGTVTLTCASSTGAV | |
| TSGYYPNWFQQKPGQAPRALIYSTSNKHSWTPARFSGSLLGGK | |
| AALTLSGVQPEDEAEYYCLLYYGGAQWVFGGGTKLTVL (SEQ | |
| ID NO: 360) | |
| EVQLVQSGAEVKKPGESLKISCKGSGYSFTSYWIGWVRQMPGK | 25 |
| GLEWMGIIYPGDSDTRYSPSFQGQVTISADKSISTAYLQWSSLK | |
| ASDTAMYYCARERDRWFDPWGQGTLVTVSSGGGGSGGGGSG | |
| GGGSGGDIQMTQSPSSLSASVGDRVTITCRASQSISSYLNWYQQ | |
| KPGKAPKLLIYAASSLQSGVPSRFSGSGSGTDFTLTISSLQPEDF | |
| ATYYCQQSYSTPLTFGGGTKVEIK (SEQ ID NO: 361) | |
| QVQLVQSGAEVKKPGASVKVSCKASGYTFTSYGISWVRQAPG | 26 |
| QGLEWMGWISAYNGNTNYAQKLQGRVTMTTDTSTSTAYMEL | |
| RSLRSDDTAVYYCARETPPSLGAFDIWGQGTMVTVSSGGGGSG | |
| GGGSGGGGSGGQSALTQPPSASGSPGQSVTISCTGTSSDVGGYN | |
| YVSWYQQHPGKAPKLMIYEVSKRPSGVPDRFSGSKSGNTASLT | |
| VSGLQAEDEADYYCSSYAGSNNWVFGGGTKLTVL (SEQ ID | |
| NO: 362) | |
| QLQLQESGPGLVKPSETLSLTCTVSGGSISSSSYYWGWIRQPPG | 27 |
| KGLEWIGSIYYSGSTYYNPSLKSRVTISVDTSKNQFSLKLSSVTA | |
| ADTAVYYCAREAYCLSDSYWYFDLWGRGTLVTVSSGGGGSG | |
| GGGSGGGGSGGQSVLTQPPSASGTPGQRVTISCSGSSSNIGSNT | |
| VNWYQQLPGTAPKLLIYSNNQRPSGVPDRFSGSKSGTSASLAIS | |
| GLQSEDEADYYCAAWDDSLNGWVFGGGTKLTVL (SEQ ID NO: | |
| 363) | |
| QVQLQESGPGLVKPSQTLSLTCTVSGGSISSGGYYWSWIRQPPG | 28 |
| KGLEWIGYIYYSGSTYYNPSLKSRVTISVDTSKNQFSLKLSSVTA | |
| ADTAVYYCARESWKYFYPRGYMDVWGKGTTVTVSSGGGGSG | |
| GGGSGGGGSGGDIQMTQSPSSLSASVGDRVTITCRASQSISSYL | |
| NWYQQKPGKAPKLLIYAASSLQSGVPSRFSGSGSGTDFTLTISSL | |
| QPEDFATYYCQQSYSTPLTFGGGTKVEIK (SEQ ID NO: 364) | |
| EVKLEESGGGLVQPGGSMKLSCVASGFTFSNYWMNWVRQSPE | GAP A3 |
| KGLEWVAEIRLKSTNYATHYAESVKGRFTISRDDSKSSVYLQM | |
| NNLRAEDTGIYYCTTLITPDYWGQGTTLTVSSGGGGSGGGGSG | |
| GGGSGGDIVMTQSHKFMSTSVGDRVSITCKASQDVSTTVAWY | |
| QQKPGQSPKLLIYSASYRYTGVPDRFTGSGSGTDFTFTISSVQAE | |
| DLAVYYCQQHYSTPPTFGGGTKLEIK (SEQ ID NO: 21956) | |

HLA-A*01 antigen binding domains

| | |
|---|---|
| QVQLVQSGAEVKKPGASVKVSCKASGYTFTSYGISWVRQAPG | A1-9 |
| QGLEWMGWISAYNGNTNYAQKLQGRVTMTTDTSTSTAYMEL | |
| RSLRSDDTAVYYCARGGWTAWYYYMDVWGKGTTVTVSSGG | |
| GGSGGGGSGGGGSGGQTVVTQEPSLTVSPGGTVTLTCASSTGA | |
| VTSGYYPNWFQQKPGQAPRALIYSTSNKHSWTPARFSGSLLGG | |
| KAALTLSGVQPEDEAEYYCLLYYGGAQWVFGGGTKLTVL | |
| (SEQ ID NO: 365) | |
| EVQLVESGGGLVQPGGSLRLSCAASGFTFSSYSMNWVRQAPGK | A1-8 |
| GLEWVSYISSSSSTIYYADSVKGRFTISRDNAKNSLYLQMNSLR | |
| AEDTAVYYCARAKYYYMDVWGKGTTVTVSSGGGGSGGGGSG | |
| GGGSGGQSVLTQPPSASGTPGQRVTISCSGSSSNIGSNTVNWYQ | |
| QLPGTAPKLLIYSNNQRPSGVPDRFSGSKSGTSASLAISGLQSED | |
| EADYYCAAWDDSLNGWVFGGGTKLTVL (SEQ ID NO: 366) | |
| QVQLQESGPGLVKPSQTLSLTCTVSGGSISSGGYYWSWIRQPPG | A1-7 |
| KGLEWIGYIYYSGSTYYNPSLKSRVTISVDTSKNQFSLKLSSVTA | |
| ADTAVYYCARDQVDKNTYYYYMDVWGKGTTVTVSSGGGGS | |
| GGGGSGGGGSGGDIQMTQSPSSLSASVGDRVTITCRASQSISSY | |
| LNWYQQKPGKAPKLLIYAASSLQSGVPSRFSGSGSGTDFTLTISS | |
| LQPEDFATYYCQQSYSTPLTFGGGTKVEIK (SEQ ID NO: 367) | |
| QVQLVESGGGLVKPGGSLRLSCAASGFTFSDYYMSWIRQAPGK | A1-6 |
| GLEWVSYISSSGSTIYYADSVKGRFTISRDNAKNSLYLQMNSLR | |
| AEDTAVYYCARACQLAEYFQHWGQGTLVTVSSGGGGSGGGG | |
| SGGGGSGGDIQMTQSPSSVSASVGDRVTITCRASQGISSWLAW | |
| YQQKPGKAPKLLIYAASSLQSGVPSRFSGSGSGTDFTLTISSLQP | |
| EDFATYYCQQANSFPLTFGGGTKVEIK (SEQ ID NO: 368) | |
| QVQLQESGPGLVKPSQTLSLTCTVSGGSISSGGYYWSWIRQPPG | A1-5 |
| KGLEWIGYIYYSGSTYYNPSLKSRVTISVDTSKNQFSLKLSSVTA | |
| ADTAVYYCARDRVDKNTSYYYMDVWGKGTTVTVSSGGGGSG | |
| GGGSGGGGSGGDIQMTQSPSSLSASVGDRVTITCRASQSISSYL | |
| NWYQQKPGKAPKLLIYAASSLQSGVPSRFSGSGSGTDFTLTISSL | |
| QPEDFATYYCQQSYSTPLTFGGGTKVEIK (SEQ ID NO: 369) | |
| QVQLQESGPGLVKPSDTLSLTCAVSGYSISSSNWWGWIRQPPG | A1-4 |
| KGLEWIGYIYYSGSTYYNPSLKSRVTMSVDTSKNQFSLKLSSVT | |
| AVDTAVYYCARRVQLKLVHWFDPWGQGTLVTVSSGGGGSGG | |
| GGSGGGGSGGDIQMTQSPSSLSASVGDRVTITCRASQSISSYLN | |
| WYQQKPGKAPKLLIYAASSLQSGVPSRFSGSGSGTDFTLTISSLQ | |
| PEDFATYYCQQSYSTPLTFGGGTKVEIK (SEQ ID NO: 370) | |
| QVQLVQSGAEVKKPGASVKVSCKASGYTFTSYDINWVRQATG | A1-3 |
| QGLEWMGWMNPNSGNTGYAQKFQGRVTMTRNTSISTAYMEL | |
| SSLRSEDTAVYYCATYYDYVTVFYFQHWGQGTLVTVSSGGGG | |
| SGGGGSGGGGSGGDIQMTQSPSSLSASVGDRVTITCRASQSISS | |
| YLNWYQQKPGKAPKLLIYAASSLQSGVPSRFSGSGSGTDFTLTI | |
| SSLQPEDFATYYCQQSYSTPLTFGGGTKVEIK (SEQ ID NO: 371) | |
| QLQLQESGSGLVKPSQTLSLTCAVSGGSISSGGYSWSWIRQPPG | A1-2 |
| KGLEWIGYIYHSGSTYYNPSLKSRVTISVDRSKNQFSLKLSSVT | |
| AADTAVYYCARESYPSFYAFDIWGQGTMVTVSSGGGGSGGGG | |
| SGGGGSGGDIQMTQSPSSLSASVGDRVTITCRASQSISSYLNWY | |
| QQKPGKAPKLLIYAASSLQSGVPSRFSGSGSGTDFTLTISSLQPE | |
| DFATYYCQQSYSTPLTFGGGTKVEIK (SEQ ID NO: 372) | |
| QITLKESGPTLVKPTQTLTLTCTFSGFSLSTSGVGVGWIRQPPGK | A1-1 |
| ALEWLALIYWNDDKRYSPSLKSRLTITKDTSKNQVVLTMTNM | |
| DPVDTATYYCAHSNMWSYSLNDYYFDYWGQGTLVTVSSGGG | |
| GSGGGGSGGGGSGGDIQMTQSPSSLSASVGDRVTITCRASQSIS | |
| SYLNWYQQKPGKAPKLLIYAASSLQSGVPSRFSGSGSGTDFTLT | |
| ISSLQPEDFATYYCQQSYSTPLTFGGGTKVEIK (SEQ ID NO: 373) | |

In some embodiments, the ligand binding domain of the second, inhibitory receptor comprises an scFv. In some embodiments, the scFv binds to HLA-A*01, HLA-A*02, HLA-A*03, HLA-A*11, HLA-B*07 or HLA-C*07, and comprises a sequence selected from the sequences set forth in Tables 3 and 4, or a sequence having at least 80%, at least 85%, at least 90%, at least 95%, at least 97% or at least 99% identity thereto. In some embodiments, the scFv binds to HLA-A*01, HLA-A*02, HLA-A*03, HLA-A*11, HLA-B*07 or HLA-C*07, and comprises a sequence selected from the group of sequences set forth in Tables 3 and 4. In some embodiments, the non-target antigen comprises HLA-A*01, and the non-target extracellular ligand binding domain of the second receptor comprises an HLA-A*01 scFv sequence comprising SEQ ID NOS: 365-373 as set forth in Table 4, or a sequence having at least 80%, at least 85%, at least 90%, at least 95%, at least 97% or at least 99% identity thereto. In some embodiments, the non-target antigen comprises HLA-A*02, and the non-target extracellular ligand binding domain of the second receptor comprises an HLA-A*02 scFv sequence comprising SEQ ID NOS: 63-74, 207, 209 or 211 as set forth in Table 3 or 4, or a sequence having at least 80%, at least 85%, at least 90%, at least 95%, at least 97% or at least 99% identity thereto. In some embodiments, the non-target antigen comprises HLA-A*03, and the non-target extracellular ligand binding domain of the second receptor comprises an HLA-A*03 scFv sequence comprising SEQ ID NOS: 351-364 as set forth in Table 4, or a sequence having at least 80%, at least 85%, at least 90%, at least 95%, at least 97% or at least 99% identity thereto. In some embodiments, the non-target antigen comprises HLA-A*11, and the non-target extracellular ligand binding domain of the second receptor comprises an HLA-A*11 scFv sequence comprising SEQ ID NOS: 52-60 as set forth in Table 4, or a sequence having at least 80%, at least 85%, at least 90%, at least 95%, at least 97% or at least 99% identity thereto. In some embodiments, the non-target antigen comprises HLA-B*07, and the non-target extracellular ligand binding domain of the second receptor comprises an HLA-B*07 scFv sequence comprising SEQ ID NOS: 296-305 as set forth in Table 4, or a sequence having at least 80%, at least 85%, at least 90%, at least 95%, at least 97% or at least 99% identity thereto. In some embodiments, the non-target antigen comprises HLA-C*07, and the non-target extracellular ligand binding domain of the second receptor comprises an HLA-C*07 scFv sequence comprising SEQ ID NOS: 306-350 as set forth in Table 4, or a sequence having at least 80%, at least 85%, at least 90%, at least 95%, at least 97% or at least 99% identity thereto.

Exemplary heavy chain and light chain CDRs (CDR-H1, CDR-H2 and CDR-H3, or CDR-L1, CDR-L2 and CDR-L3, respectively) for HLA antigen binding domains are shown in Table 5 below.

TABLE 5
HLA CDR Sequences

| | | | | | |
|---|---|---|---|---|---|
| CDR-L1 | CDR-L2 | CDR-L3 | CDR-H1 | CDR-H2 | CDR-H3 |

HLA-A*02 CDRs

| | | | | | |
|---|---|---|---|---|---|
| RSSQSIVHS | KVSNRF | FQGSHVP | ASGYTFT | WIYPGNVNT | EEITYAMDY |
| NGNTYLE | SGVPDR | RT (SEQ | SYHIH | EYNEKFKGK | (SEQ ID NO: |
| (SEQ ID NO: | (SEQ ID | ID NO: 92) | (SEQ ID | (SEQ ID NO: | 101) |
| 87) | NO: 90) | | NO: 95) | 98) | |
| RSSQSIVHS | KVSNRF | FQGSHVP | ASGYTFT | WIYPGNVNT | EEITYAMDY |
| NGNTYLE | SGVPAR | RT (SEQ | SYHIH | EYNEKFKGK | (SEQ ID NO: |
| (SEQ ID NO: | (SEQ ID | ID NO: 92) | (SEQ ID | (SEQ ID NO: | 101) |
| 87) | NO: 91) | | NO: 95) | 98) | |
| SSSQSIVHS | KVSNRFS | HQGSHVP | ASGYTFT | YIYPGNVNT | EEITYAMDY |
| NGNTYME | GVPDR | RT (SEQ | SYHMH | EYNEKFKGK | (SEQ ID NO: |
| (SEQ ID NO: | (SEQ ID | ID NO: 93) | (SEQ ID | (SEQ ID NO: | 101) |
| 88) | NO: 90) | | NO: 96) | 99) | |
| RSSQSIVHS | KVSNRF | MQGSHV | SGYTFTS | WIYPGDGST | EGTYYAMDY |
| NGNTYLD | SGVPDR | PRT (SEQ | YHMH | QYNEKFKG | (SEQ ID NO: |
| (SEQ ID NO: | (SEQ ID | ID NO: 94) | (SEQ ID | (SEQ ID NO: | 102) |
| 89) | NO: 90) | | NO: 97) | 100) | |
| RSSQSIVHS | KVSNRF | FQGSHVP | SYHIH | WIYPGNVNT | EEITYAMDY |
| NGNTYLE | S (SEQ ID | RT (SEQ | (SEQ ID | EYNEKFKG | (SEQ ID NO: |
| (SEQ ID NO: | NO: | ID NO: 92) | NO: | (SEQ ID NO: | 101) |
| 87) | 21985) | | 21986) | 21987) | |

HLA-A *03 CDRs

| | | | | | |
|---|---|---|---|---|---|
| RASQSISSY | AASSLQ | QQSYSTP | SYGIS | WISAYNGNT | ERVSQRGAFD |
| LN (SEQ ID | S (SEQ ID | LT (SEQ | (SEQ ID | NYAQKLQG | I (SEQ ID NO: |
| NO: 29) | NO: 30) | ID NO: 31) | NO: 374) | (SEQ ID NO: | 376) |
| | | | | 375) | |
| RASQSISSY | AASSLQ | QQSYSTP | SYSMN | YISSSSSTIYY | GNPDKDPFDY |
| LN (SEQ ID | S (SEQ ID | LT (SEQ | (SEQ ID | ADSVKG | (SEQ ID NO: |
| NO: 29) | NO: 30) | ID NO: 31) | NO: 377) | (SEQ ID NO: | 379) |
| | | | | 378) | |
| RASQSISSY | AASSLQ | QQSYSTP | SGSYYW | YIYYSGSTN | DFYCTNWYFD |
| LN (SEQ ID | S (SEQ ID | LT (SEQ | S (SEQ ID | YNPSLKS | L (SEQ ID NO: |
| NO: 29) | NO: 30) | ID NO: 31) | NO: 380) | (SEQ ID NO: | 382) |
| | | | | 381) | |
| RASQSISSY | AASSLQ | QQSYSTP | SYYWS | YIYYSGSTN | ESSSGSYWYF |
| LN (SEQ ID | S (SEQ ID | LT (SEQ | (SEQ ID | YNPSLKS | DL (SEQ ID NO: |
| NO: 29) | NO: 30) | ID NO: 31) | NO: 383) | (SEQ ID NO: | 384) |
| | | | | 381) | |
| RASQSISSY | AASSLQ | QQSYSTP | SYWIG | IIYPGDSDTR | DSGYKYNLYY |
| LN (SEQ ID | S (SEQ ID | LT (SEQ | (SEQ ID | YSPSFQG | YYYYMDV |
| NO: 29) | NO: 30) | ID NO: 31) | NO: 385) | (SEQ ID NO: | (SEQ ID NO: |
| | | | | 386) | 387) |
| ASSTGAVT | STSNKH | LLYYGG | SYGIS | WISAYNGNT | GGDLSHYYYY |
| SGYYPN | S (SEQ ID | AQWV | (SEQ ID | NYAQKLQG | MDV (SEQ ID |
| (SEQ ID NO: | NO: 389) | (SEQ ID | NO: 374) | (SEQ ID NO: | NO: 21972) |
| 388) | | NO: 390) | | 375) | |
| RASQSISSY | AASSLQ | QQSYSTP | SYGIS | WISAYNGNT | ENRRYNSCYY |
| LN (SEQ ID | S (SEQ ID | LT (SEQ | (SEQ ID | NYAQKLQG | FDY (SEQ ID |
| NO: 29) | NO: 30) | ID NO: 31) | NO: 374) | (SEQ ID NO: | NO: 391) |
| | | | | 375) | |
| ASSTGAVT | STSNKH | LLYYGG | SYGIS | WISAYNGNT | GGDLSHYYYY |
| SGYYPN | S (SEQ ID | AQWV | (SEQ ID | NYAQKLQG | LDV (SEQ ID |
| (SEQ ID NO: | NO: 389) | (SEQ ID | NO: 374) | (SEQ ID NO: | NO: 392) |
| 388) | | NO: 390) | | 375) | |
| RASQSISSY | AASSLQ | QQSYSTP | SNYMS | VIYSGGSTY | ATLLSLSYDA |
| LN (SEQ ID | S (SEQ ID | LT (SEQ | (SEQ ID | YADSVKG | FDI (SEQ ID |
| NO: 29) | NO: 30) | ID NO: 31) | NO: 393) | (SEQ ID NO: | NO: 395) |
| | | | | 394) | |
| ASSTGAVT | STSNKH | LLYYGG | SYGIS | WISAYNGNT | GGDLSHYYY |
| SGYYPN | S (SEQ ID | AQWV | (SEQ ID | NYAQKLQG | MDV (SEQ ID |
| (SEQ ID NO: | NO: 389) | (SEQ ID | NO: 374) | (SEQ ID NO: | NO: 396) |
| 388) | | NO: 390) | | 375) | |
| RASQSISSY | AASSLQ | QQSYSTP | SYWIG | IIYPGDSDTR | ERDRWFDP |
| LN (SEQ ID | S (SEQ ID | LT (SEQ | (SEQ ID | YSPSFQG | (SEQ ID NO: |
| NO: 29) | NO: 30) | ID NO: 31) | NO: 385) | (SEQ ID NO: | 397) |
| | | | | 386) | |
| TGTSSDVG | EVSKRP | SSYAGSN | SYGIS | WISAYNGNT | ETPPSLGAFDI |
| GYNYVS | S (SEQ ID | NWV | (SEQ ID | NYAQKLQG | (SEQ ID NO: |
| (SEQ ID NO: | NO: 399) | (SEQ ID | NO: 374) | (SEQ ID NO: | 401) |
| 398) | | NO: 400) | | 375) | |
| SGSSSNIGS | SNNQRP | AAWDDS | SSSYYW | SIYYSGSTYY | EAYCLSDSYW |
| NTVN (SEQ | S (SEQ ID | LNGWV | G (SEQ ID | NPSLKS (SEQ | YFDL (SEQ ID |
| ID NO: 402) | NO: 403) | (SEQ ID | NO: 405) | ID NO: 406) | NO: 407) |
| | | NO: 404) | | | |
| RASQSISSY | AASSLQ | QQSYSTP | SGGYYW | YIYYSGSTY | ESWKYFYPRG |
| LN (SEQ ID | S (SEQ ID | LT (SEQ | S (SEQ ID | YNPSLKS | YMDV (SEQ ID |
| NO: 29) | NO: 30) | ID NO: 31) | NO: 10) | (SEQ ID NO: | NO: 408) |
| | | | | 15) | |
| KASQDVST | SASYRY | QQHYSTP | NYWMN | EIRLKSTNY | LITPDY (SEQ |
| TVA (SEQ | T (SEQ | PT (SEQ | (SEQ ID | ATHYAESVK | ID NO: 21962) |
| ID NO: | ID NO: | ID NO: | NO: | G (SEQ ID | |
| 21957) | 21958) | 21959) | 21960) | NO: 21961) | |

HLA-B*07 CDRS

| | | | | | |
|---|---|---|---|---|---|
| RASENIYS | AATYLP | QHFWVT | SGYSWH | YIHFSGSTHY | GGVVSHYAM |
| NLA (SEQ | D (SEQ | PYT (SEQ | (SEQ ID | HPSLKS (SEQ | DC (SEQ ID NO: |
| ID NO: 409) | ID NO: | ID NO: | NO: 412) | ID NO: 413) | 414) |
| | 410) | 411) | | | |

HLA-A*11 CDRs

| | | | | | |
|---|---|---|---|---|---|
| RASQSISSY | AASSLQ | QQSYSTP | SGGYYW | YIYYSGSTY | HYYYYYMDV |
| LN (SEQ ID | S (SEQ ID | LT (SEQ | S (SEQ ID | YNPSLKS | (SEQ ID NO: 23) |
| NO: 29) | NO: 30) | ID NO: 31) | NO: 10) | (SEQ ID NO: | |
| | | | | 15) | |
| RASQSISSY | AASSLQ | QQSYSTP | TSGVGV | LIYWNDDKR | KTTSFYFDY |
| LN (SEQ ID | S (SEQ ID | LT (SEQ | G (SEQ ID | YSPSLKS | (SEQ ID NO: 24) |
| NO: 29) | NO: 30) | ID NO: 31) | NO: 11) | (SEQ ID NO: | |
| | | | | 16) | |
| RASQSISSY | AASSLQ | QQSYSTP | SGGYYW | YIYYSGSTY | HYYYYMDV |
| LN (SEQ ID | S (SEQ ID | LT (SEQ | S (SEQ ID | YNPSLKS | (SEQ ID NO: 22) |
| NO: 29) | NO: 30) | ID NO: 31) | NO: 10) | (SEQ ID NO: | |
| | | | | 15) | |
| RASQSISSY | AASSLQ | QQSYSTP | SYWMH | RINSDGSSTS | GVLLYNWFDP |
| LN (SEQ ID | S (SEQ ID | LT (SEQ | (SEQ ID | YADSVKG | (SEQ ID NO: 28) |
| NO: 29) | NO: 30) | ID NO: 31) | NO: 14) | (SEQ ID NO: | |
| | | | | 19) | |
| RASQSISSY | AASSLQ | QQSYSTP | SGGYYW | YIYYSGSTY | HYYYYYLDV |
| LN (SEQ ID | S (SEQ ID | LT (SEQ | S (SEQ ID | YNPSLKS | (SEQ ID NO: 21) |
| NO: 29) | NO: 30) | ID NO: 31) | NO: 10) | (SEQ ID NO: | |
| | | | | 15) | |
| RASQSISSY | AASSLQ | QQSYSTP | SYDMH | AIGTAGDTY | DLPGSYWYFD |
| LN (SEQ ID | S (SEQ ID | LT (SEQ | (SEQ ID | YPGSVKG | L (SEQ ID NO: |
| NO: 29) | NO: 30) | ID NO: 31) | NO: 13) | (SEQ ID NO: | 27) |
| | | | | 18) | |
| RASQSISSY | AASSLQ | QQSYSTP | SYAMH | WINAGNGNT | EGNGANPDAF |
| LN (SEQ ID | S (SEQ ID | LT (SEQ | (SEQ ID | KYSQKFQG | DI (SEQ ID NO: |
| NO: 29) | NO: 30) | ID NO: 31) | NO: 12) | (SEQ ID NO: | 26) |
| | | | | 17) | |
| RASQSISSY | AASSLQ | QQSYSTP | TSGVGV | LIYWNDDKR | RHMRLSCFDY |
| LN (SEQ ID | S (SEQ ID | LT (SEQ | G (SEQ ID | YSPSLKS | (SEQ ID NO: 25) |
| NO: 29) | NO: 30) | ID NO: 31) | NO: 11) | (SEQ ID NO: | |
| | | | | 16) | |
| RASQSISSY | AASSLQ | QQSYSTP | SGGYYW | YIYYSGSTY | HYYYYSMDV |
| LN (SEQ ID | S (SEQ ID | LT (SEQ | S (SEQ ID | YNPSLKS | (SEQ ID NO: 20) |
| NO: 29) | NO: 30) | ID NO: 31) | NO: 10) | (SEQ ID NO: | |
| | | | | 15) | |

HLA-C*07 CDRs

| | | | | | |
|---|---|---|---|---|---|
| RASQSISSY | AASSLQ | QQSYSTP | SYAMS | AISGSGGSTY | SFDWFDP |
| LN (SEQ ID | S (SEQ ID | LT (SEQ | (SEQ ID | YADSVKG | (SEQ ID NO: |
| NO: 29) | NO: 30) | ID NO: 31) | NO: 415) | (SEQ ID NO: | 417) |
| | | | | 416) | |
| RASQSISSY | AASSLQ | QQSYSTP | SGGYYW | YIYYSGSTY | ERSISPYYYYY |
| LN (SEQ ID | S (SEQ ID | LT (SEQ | S (SEQ ID | YNPSLKS | MDV (SEQ ID |
| NO: 29) | NO: 30) | ID NO: 31) | NO: 10) | (SEQ ID NO: | NO: 418) |
| | | | | 15) | |
| SGSSSNIGS | SNNQRP | AAWDDS | SSSYYW | SIYYSGSTYY | DSVIWYWFDP |
| NTVN (SEQ | S (SEQ ID | LNGWV | G (SEQ ID | NPSLKS (SEQ | (SEQ ID NO: |
| ID NO: 402) | NO: 403) | (SEQ ID | NO: 405) | ID NO: 406) | 419) |
| | | NO: 404) | | | |
| RASQSISSY | AASSLQ | QQSYSTP | SGGYYW | YIYYSGSTY | EEILPRLSYYY |
| LN (SEQ ID | S (SEQ ID | LT (SEQ | S (SEQ ID | YNPSLKS | YMDV (SEQ ID |
| NO: 29) | NO: 30) | ID NO: 31) | NO: 10) | (SEQ ID NO: | NO: 420) |
| | | | | 15) | |
| RASQSISSY | AASSLQ | QQSYSTP | SYAMN | WINTNTGNP | GGRAHSSWYF |
| LN (SEQ ID | S (SEQ ID | LT (SEQ | (SEQ ID | TYAQGFTG | DL (SEQ ID NO: |
| NO: 29) | NO: 30) | ID NO: 31) | NO: 421) | (SEQ ID NO: | 423) |
| | | | | 422) | |
| RASQSISSY | AASSLQ | QQSYSTP | SGGYYW | YIYYSGSTY | DRIKILPRLGY |
| LN (SEQ ID | S (SEQ ID | LT (SEQ | S (SEQ ID | YNPSLKS | YYYMDV (SEQ |
| NO: 29) | NO: 30) | ID NO: 31) | NO: 10) | (SEQ ID NO: | ID NO: 424) |
| | | | | 15) | |
| RASQSISSY | AASSLQ | QQSYSTP | SGGYYW | YIYYSGSTY | DTVIHYYYYM |
| LN (SEQ ID | S (SEQ ID | LT (SEQ | S (SEQ ID | YNPSLKS | DV (SEQ ID |
| NO: 29) | NO: 30) | ID NO: 31) | NO: 10) | (SEQ ID NO: | NO: 425) |
| | | | | 15) | |
| RASQSISSY | AASSLQ | QQSYSTP | SGGYYW | YIYYSGSTY | DVIVEVFLSY |
| LN (SEQ ID | S (SEQ ID | LT (SEQ | S (SEQ ID | YNPSLKS | YYYMDV (SEQ |
| NO: 29) | NO: 30) | ID NO: 31) | NO: 10) | (SEQ ID NO: | ID NO: 426) |
| | | | | 15) | |
| RASQSISSY | AASSLQ | QQSYSTP | SGGYYW | YIYYSGSTY | DIFIHYYYYM |
| LN (SEQ ID | S (SEQ ID | LT (SEQ | S (SEQ ID | YNPSLKS | DV (SEQ ID |
| NO: 29) | NO: 30) | ID NO: 31) | NO: 10) | (SEQ ID NO: | NO: 427) |
| | | | | 15) | |
| RASQSISSY | AASSLQ | QQSYSTP | SYSMN | YISSSSSTIYY | DGTFYSYSPY |
| LN (SEQ ID | S (SEQ ID | LT (SEQ | (SEQ ID | ADSVKG | YFDY (SEQ ID |
| NO: 29) | NO: 30) | ID NO: 31) | NO: 377) | (SEQ ID NO: | NO: 428) |
| | | | | 378) | |
| RASQSISSY | AASSLQ | QQSYSTP | SGGYYW | YIYYSGSTY | EWIKILPRLGY |
| LN (SEQ ID | S (SEQ ID | LT (SEQ | S (SEQ ID | YNPSLKS | YYYMDV (SEQ |
| NO: 29) | NO: 30) | ID NO: 31) | NO: 10) | (SEQ ID NO: | ID NO: 429) |
| | | | | 15) | |
| RASQSISSY | AASSLQ | QQSYSTP | SGGYYW | YIYYSGSTY | DRSLYYYYY |
| LN (SEQ ID | S (SEQ ID | LT (SEQ | S (SEQ ID | YNPSLKS | MDV (SEQ ID |
| NO: 29) | NO: 30) | ID NO: 31) | NO: 10) | (SEQ ID NO: | NO: 430) |
| | | | | 15) | |
| RASQSISSY | AASSLQ | QQSYSTP | SGGYYW | YIYYSGSTY | DKILAPNYYY |
| LN(SEQ ID | S (SEQ ID | LT (SEQ | S (SEQ ID | YNPSLKS | YMDV (SEQ ID |
| NO: 29) | NO: 30) | ID NO: 31) | NO: 10) | (SEQ ID NO: | NO: 431) |
| | | | | 15) | |
| RASQSISSY | AASSLQ | QQSYSTP | SGGYYW | YIYYSGSTY | EKSWKYFYY |
| LN (SEQ ID | S (SEQ ID | LT (SEQ | S (SEQ ID | YNPSLKS | YYYYMDV |
| NO: 29) | NO: 30) | ID NO: 31) | NO: 10) | (SEQ ID NO: | (SEQ ID NO: |
| | | | | 15) | 432) |
| RASQSISSY | AASSLQ | QQSYSTP | SGGYYW | YIYYSGSTY | ENTSTIPYYYY |
| LN (SEQ ID | S (SEQ ID | LT (SEQ | S (SEQ ID | YNPSLKS | YMDV (SEQ ID |
| NO: 29) | NO: 30) | ID NO: 31) | NO: 10) | (SEQ ID NO: | NO: 433) |
| | | | | 15) | |
| RASQSISSY | AASSLQ | QQSYSTP | SGGYYW | YIYYSGSTY | EDVDKNTSTI |
| LN (SEQ ID | S (SEQ ID | LT (SEQ | S (SEQ ID | YNPSLKS | YYYYYYMDV |
| NO: 29) | NO: 30) | ID NO: 31) | NO: 10) | (SEQ ID NO: | (SEQ ID NO: |
| | | | | 15) | 434) |
| RASQGISS | DASSLES | QQFNSYP | DYYMS | YISSSGSTIY | DGGDIVSSSAI |
| ALA (SEQ | (SEQ ID | LT (SEQ | (SEQ ID | YADSVKG | YWYFDL (SEQ |
| ID NO: 435) | NO: 436) | ID NO: | NO: 438) | (SEQ ID NO: | ID NO: 440) |
| | | 437) | | 439) | |
| RASQSISSY | AASSLQ | QQSYSTP | SGGYYW | YIYYSGSTY | DLILPPYYYY |
| LN (SEQ ID | S (SEQ ID | LT (SEQ | S (SEQ ID | YNPSLKS | YMDV (SEQ ID |
| NO: 29) | NO: 30) | ID NO: 31) | NO: 10) | (SEQ ID NO: | NO: 441) |
| | | | | 15) | |
| RASQSISSY | AASSLQ | QQSYSTP | SGGYYW | YIYYSGSTY | ETWIKILPRYY |
| LN (SEQ ID | S (SEQ ID | LT (SEQ | S (SEQ ID | YNPSLKS | YYYYYMDV |
| NO: 29) | NO: 30) | ID NO: 31) | NO: 10) | (SEQ ID NO: | (SEQ ID NO: |
| | | | | 15) | 442) |
| RASQSISSY | AASSLQ | QQSYSTP | SGGYYW | YIYYSGSTY | DLSRYYYYY |
| LN (SEQ ID | S (SEQ ID | LT (SEQ | S (SEQ ID | YNPSLKS | MDV (SEQ ID |
| NO: 29) | NO: 30) | ID NO: 31) | NO: 10) | (SEQ ID NO: | NO: 443) |
| | | | | 15) | |
| RASQGISS | AASSLQ | QQYNSYP | SYSMN | YISSSSSTIYY | EHIVLCFDY |
| WLA (SEQ | S (SEQ ID | LT (SEQ | (SEQ ID | ADSVKG | (SEQ ID NO: |
| ID NO: 444) | NO: 30) | ID NO: | NO: 377) | (SEQ ID NO: | 446) |
| | | 445) | | 378) | |
| RASQSISSY | AASSLQ | QQSYSTP | SGGYYW | YIYYSGSTY | DKILPRPYYY |
| LN (SEQ ID | S (SEQ ID | LT (SEQ | S (SEQ ID | YNPSLKS | YYMDV (SEQ |
| NO: 29) | NO: 30) | ID NO: 31) | NO: 10) | (SEQ ID NO: | ID NO: 447) |
| | | | | 15) | |
| TGTSSDVG | EVSKRP | SSYAGSN | SYGIS | WISAYNGNT | GSNEYFQH |
| GYNYVS | S (SEQ ID | NWV | (SEQ ID | NYAQKLQG | (SEQ ID NO: |
| (SEQ ID NO: | NO: 399) | (SEQ ID | NO: 374) | (SEQ ID NO: | 448) |
| 398) | | NO: 400) | | 375) | |
| RASQSISSY | AASSLQ | QQSYSTP | SYAMN | WINTNTGNP | GTSYWYFDL |
| LN (SEQ ID | S (SEQ ID | LT (SEQ | (SEQ ID | TYAQGFTG | (SEQ ID NO: |
| NO: 29) | NO: 30) | ID NO: 31) | NO: 421) | (SEQ ID NO: | 449) |
| | | | | 422) | |
| RASQSISSY | AASSLQ | QQSYSTP | SGGYYW | YIYYSGSTY | EEIVEVFYYY |
| LN (SEQ ID | S (SEQ ID | LT (SEQ | S (SEQ ID | YNPSLKS | YMDV (SEQ ID |
| NO: 29) | NO: 30) | ID NO: 31) | NO: 10) | (SEQ ID NO: | NO: 450) |
| | | | | 15) | |
| RASQSISSY | AASSLQ | QQSYSTP | SYAMS | AISGSGGSTY | VDDYYFDY |
| LN (SEQ ID | S (SEQ ID | LT (SEQ | (SEQ ID | YADSVKG | (SEQ ID NO: |
| NO: 29) | NO: 30) | ID NO: 31) | NO: 415) | (SEQ ID NO: | 451) |
| | | | | 416) | |
| RASQSISSY | AASSLQ | QQSYSTP | SYWMH | RINSDGSSTS | STNILLSYTKA |
| LN (SEQ ID | S (SEQ ID | LT (SEQ | (SEQ ID | YADSVKG | FDI (SEQ ID |
| NO: 29) | NO: 30) | ID NO: 31) | NO: 14) | (SEQ ID NO: | NO: 452) |
| | | | | 19) | |
| RASQSISSY | AASSLQ | QQSYSTP | SGGYYW | YIYYSGSTY | DKTYYYYYY |
| LN (SEQ ID | S (SEQ ID | LT (SEQ | S (SEQ ID | YNPSLKS | MDV (SEQ ID |
| NO: 29) | NO: 30) | ID NO: 31) | NO: 10) | (SEQ ID NO: | NO: 453) |
| | | | | 15) | |
| RASQSISSY | AASSLQ | QQSYSTP | SGGYYW | YIYYSGSTY | EKYFHDKYFH |
| LN (SEQ ID | S (SEQ ID | LT (SEQ | S (SEQ ID | YNPSLKS | DYYYYYMDV |
| NO: 29) | NO: 30) | ID NO: 31) | NO: 10) | (SEQ ID NO: | (SEQ ID NO: |
| | | | | 15) | 454) |
| RASQSISSY | AASSLQ | QQSYSTP | SGGYYW | YIYYSGSTY | DTSVYYYYY |
| LN (SEQ ID | S (SEQ ID | LT (SEQ | S (SEQ ID | YNPSLKS | MDV (SEQ ID |
| NO: 29) | NO: 30) | ID NO: 31) | NO: 10) | (SEQ ID NO: ) | NO: 455) |
| RASQSISSY | AASSLQ | QQSYSTP | SGGYYW | YIYYSGSTY | EKILPYYYYY |
| LN( SEQ ID | S (SEQ ID | LT (SEQ | S (SEQ ID | YNPSLKS | YMDV (SEQ ID |
| NO: 29) | NO: 30) | ID NO: 31) | NO: 10) | (SEQ ID NO: | NO: 456) |
| | | | | 15) | |
| SGSSSNIGS | SNNQRP | AAWDDS | SYSMN | YISSSSSTIYY | QWIYIYINPRG |
| NTVN (SEQ | S (SEQ ID | LNGWV | (SEQ ID | ADSVKG | FIFLHDAFDI |
| ID NO: 402) | NO: 403) | (SEQ ID | NO: 377) | (SEQ ID NO: | (SEQ ID NO: |
| | | NO: 404) | | 378) | 457) |
| RASQSISSY | AASSLQ | QQSYSTP | SNSAAW | RTYYRSKW | EDVDFHHDAF |
| LN (SEQ ID | S (SEQ ID | LT (SEQ | N (SEQ ID | YNDYAVSV | DI (SEQ ID NO: |
| NO: 29) | NO: 30) | ID NO: 31) | NO:22006) | KS (SEQ ID | 458) |
| | | | | NO: 22007) | |
| RASQSISSY | AASSLQ | QQSYSTP | SGGYYW | YIYYSGSTY | EGVDKNTSTI |
| LN (SEQ ID | S (SEQ ID | LT (SEQ | S (SEQ ID | YNPSLKS | YYYYYYMDV |
| NO: 29) | NO: 30) | ID NO: 31) | NO: 10) | (SEQ ID NO: | (SEQ ID NO: |
| | | | | 15) | 459) |
| RASQGISS | AASSLQ | QQYNSYP | SYSMN | YISSSSSTIYY | DRRGYFDL |
| WLA (SEQ | S (SEQ ID | LT (SEQ | (SEQ ID | ADSVKG | (SEQ ID NO: |
| ID NO: 444) | NO: 30) | ID NO: | NO: 377) | (SEQ ID NO: | 460) |
| | | 445) | | 378) | |
| RASQSISSY | AASSLQ | QQSYSTP | DYYMH | LVDPEDGETI | GIHVDIRSME |
| LN (SEQ ID | S (SEQ ID | LT (SEQ | (SEQ ID | YAEKFQG | DWFDP (SEQ |
| NO: 29) | NO: 30) | ID NO: 31) | NO: 461) | (SEQ ID NO: | ID NO: 463) |
| | | | | 462) | |
| RASQSISSY | AASSLQ | QQSYSTP | SGGYYW | YIYYSGSTY | DIGTSYYYYM |
| LN (SEQ ID | S (SEQ ID | LT (SEQ | S (SEQ ID | YNPSLKS | DV (SEQ ID |
| NO: 29) | NO: 30) | ID NO: 31) | NO: 10) | (SEQ ID NO: | NO: 464) |
| | | | | 15) | |
| RASQSISSY | AASSLQ | QQSYSTP | SGGYYW | YIYYSGSTY | EVVEVFLYYY |
| LN (SEQ ID | S (SEQ ID | LT (SEQ | S (SEQ ID | YNPSLKS | YYMDV (SEQ |
| NO: 29) | NO: 30) | ID NO: 31) | NO: 10) | (SEQ ID NO: | ID NO: 465) |
| | | | | 15) | |
| RASQSISSY | AASSLQ | QQSYSTP | SGGYYW | YIYYSGSTY | DLYYYYYYY |
| LN (SEQ ID | S (SEQ ID | LT (SEQ | S (SEQ ID | YNPSLKS | MDV (SEQ ID |
| NO: 29) | NO: 30) | ID NO: 31) | NO: 10) | (SEQ ID NO: | NO: 466) |
| | | | | 15) | |
| RASQSISSY | AASSLQ | QQSYSTP | SGGYYW | YIYYSGSTY | ESWKYFYPRG |
| LN (SEQ ID | S (SEQ ID | LT (SEQ | S (SEQ ID | YNPSLKS | SIFIHYYYYM |
| NO: 29) | NO: 30) | ID NO: 31) | NO: 10) | (SEQ ID NO: | DV (SEQ ID |
| | | | | 15) | NO: 467) |
| RASQSISSY | AASSLQ | QQSYSTP | SGGYYW | YIYYSGSTY | DRIVEVFYYY |
| LN (SEQ ID | S (SEQ ID | LT (SEQ | S (SEQ ID | YNPSLKS | YMDV (SEQ ID |
| NO: 29) | NO: 30) | ID NO: 31) | NO: 10) | (SEQ ID NO: | NO: 468) |
| | | | | 15) | |
| RASQSISSY | AASSLQ | QQSYSTP | SGGYYW | YIYYSGSTY | EKYFHDWLY |
| LN (SEQ ID | S (SEQ ID | LT (SEQ | S (SEQ ID | YNPSLKS | YYYYMDV |
| NO: 29) | NO: 30) | ID NO: 31) | NO: 10) | (SEQ ID NO: | (SEQ ID NO: |
| | | | | 15) | 469) |
| RASQSISSY | AASSLQ | QQSYSTP | SGGYYW | YIYYSGSTY | DLVDKNTSYY |
| LN (SEQ ID | S (SEQ ID | LT (SEQ | S (SEQ ID | YNPSLKS | YYYMDV (SEQ |
| NO: 29) | NO: 30) | ID NO: 31) | NO: 10) | (SEQ ID NO: | ID NO: 470) |
| | | | | 15) | |
| TGTSSDVG | EVSKRP | SSYAGSN | SYGIS | WISAYNGNT | VQNEYFQH |
| GYNYVS | S (SEQ ID | NWV | (SEQ ID | NYAQKLQG | (SEQ ID NO: |
| (SEQ ID NO: | NO: 399) | (SEQ ID | NO: 374) | (SEQ ID NO: | 471) |
| 398) | | NO: 400) | | 375) | |
| RASQGISS | AASSLQ | QQANSFP | DYYMS | YISSSGSTIY | ANWFDP (SEQ |
| WLA (SEQ | S (SEQ ID | LT (SEQ | (SEQ ID | YADSVKG | ID NO: 472) |
| ID NO: 444) | NO: 30) | ID NO: ) | NO: 438) | (SEQ ID NO: | |
| | | | | 439) | |

HLA-A*01 CDRs

| | | | | | |
|---|---|---|---|---|---|
| ASSTGAVT | STSNKH | LLYYGG | SYGIS | WISAYNGNT | GGWTAWYYY |
| SGYYPN | S (SEQ ID | AQWV | (SEQ ID | NYAQKLQG | MDV (SEQ ID |
| (SEQ ID NO: | NO: 389) | (SEQ ID | NO: 374) | (SEQ ID NO: | NO: 473) |
| 388) | | NO: 390) | | 375) | |
| SGSSSNIGS | SNNQRP | AAWDDS | SYSMN | YISSSSSTIYY | AKYYYMDV |
| NTVN (SEQ | S (SEQ ID | LNGWV | (SEQ ID | ADSVKG | (SEQ ID NO: |
| ID NO: 402) | NO: 403) | (SEQ ID | NO: 377) | (SEQ ID NO: | 474) |
| | | NO: 404) | | 378) | |
| RASQSISSY | AASSLQ | QQSYSTP | SGGYYW | YIYYSGSTY | DQVDKNTYY |
| LN (SEQ ID | S (SEQ ID | LT (SEQ | S (SEQ ID | YNPSLKS | YYMDV (SEQ |
| NO: ) | NO: 30) | ID NO: 31) | NO: 10) | (SEQ ID NO: | ID NO: 475) |
| | | | | 15) | |
| RASQGISS | AASSLQ | QQANSFP | DYYMS | YISSSGSTIY | ACQLAEYFQH |
| WLA (SEQ | S (SEQ ID | LT (SEQ | (SEQ ID | YADSVKG | (SEQ ID NO: |
| ID NO: 444) | NO: ) | ID NO: ) | NO: 438) | (SEQ ID NO: | 476) |
| | | | | 439) | |
| RASQSISSY | AASSLQ | QQSYSTP | SGGYYW | YIYYSGSTY | DRVDKNTSYY |
| LN (SEQ ID | S (SEQ ID | LT (SEQ | S (SEQ ID | YNPSLKS | YMDV (SEQ ID |
| NO: 29) | NO: 30) | ID NO: 31) | NO: 10) | (SEQ ID | NO: 477) |
| | | | | NO:15) | |
| RASQSISSY | AASSLQ | QQSYSTP | SSNWWG | YIYYSGSTY | RVQLKLVHW |
| LN (SEQ ID | S (SEQ ID | LT (SEQ | (SEQ | YNPSLKS | FDP (SEQ ID |
| NO: 29) | NO: 30) | ID NO: 31) | NO: 479) | (SEQ ID NO: | NO: 478) |
| | | | | 15) | |
| RASQSISSY | AASSLQ | QQSYSTP | SYDIN | WMNPNSGN | YYDYVTVFYF |
| LN (SEQ ID | S (SEQ ID | LT (SEQ | (SEQ ID | TGYAQKFQG | QH (SEQ ID |
| NO: 29) | NO: 30) | ID NO: 31) | NO: 480) | (SEQ ID NO: | NO: 482) |
| | | | | 481) | |
| RASQSISSY | AASSLQ | QQSYSTP | SGGYSW | YIYHSGSTY | ESYPSFYAFDI |
| LN (SEQ ID | S (SEQ ID | LT (SEQ | S (SEQ ID | YNPSLKS | (SEQ ID NO: |
| NO: 29) | NO: 30) | ID NO: 31) | NO: 483) | (SEQ ID NO: | 485) |
| | | | | 484) | |
| RASQSISSY | AASSLQ | QQSYSTP | TSGVGV | LIYWNDDKR | SNMWSYSLN |
| LN (SEQ ID | S (SEQ ID | LT (SEQ | G (SEQ ID | YSPSLKS | DYYFDY (SEQ |
| NO: 29) | NO: 30) | ID NO: 31) | NO: 11) | (SEQ ID NO: | ID NO: 482) |
| | | | | 16) | |

In some embodiments, the scFv comprises the complementarity determined regions (CDRs) of any one of SEQ ID NOS: 87-102. In some embodiments, the scFv comprises a sequence at least 95% identical to any one of SEQ ID NOS: 87-102. In some embodiments, the scFv comprises a sequence identical to any one of SEQ ID NOS: 87-102. In some embodiments, the heavy chain of the antibody comprises the heavy chain CDRs of any one of SEQ ID NOS: 95-102, and wherein the light chain of the antibody comprises the light chain CDRs of any one of SEQ ID NOS: 87-94. In some embodiments, the heavy chain of the antibody comprises a sequence at least 95% identical to the heavy chain portion of any one of SEQ ID NOS: 95-102, and wherein the light chain of the antibody comprises a sequence at least 95% identical to the light chain portion of any one of SEQ ID NOS: 87-94.

In some embodiments, the second, inhibitory antigen is HLA-A*02, and the inhibitory antigen binding domain comprises an HLA-A*02 antigen binding domain. In some embodiments, the second antigen binding domain binds HLA-A*02 independent of the peptide in a pMHC complex comprising HLA-A*02. In some embodiments, the HLA-A*02 antigen binding domain comprises an scFv domain. In some embodiments, the HLA-A*02 antigen binding domain comprises a sequence of any one of SEQ ID NOs: 63-74, 207, 209 or 211. In some embodiments, the HLA-A*02 antigen binding domain comprises a sequence at least 90%, at least 95% or at least 99% identical to a sequence of any one of SEQ ID NOs: 63-74, 207, 209 or 211.

In an illustrative embodiment, the non-target antigen comprises HLA-A*02, and the second inhibitory receptor comprises a sequence of SEQ ID NO: 21965, or a sequence having at least 90%, at least 95%, at least 97% or at least 99% identity thereto. In some embodiments, the non-target antigen comprises HLA-A*02 and the second inhibitory receptor comprises a sequence of SEQ ID NO: 21965.

In another illustrative embodiment, the non-target antigen comprises HLA-A*03, and the second inhibitory receptor comprises a sequence of SEQ ID NO: 21966, or a sequence having at least 90%, at least 95%, at least 97% or at least 99% identity thereto. In some embodiments, the non-target antigen comprises HLA-A*03 and the second inhibitory receptor comprises a sequence of SEQ ID NO: 21966. The corresponding nucleotide sequence comprises SEQ ID NO: 21967.

In another illustrative embodiment, the non-target antigen comprises HLA-B*07, and the second inhibitory receptor comprises a sequence of SEQ ID NO: 21970, or a sequence having at least 90%, at least 95%, at least 97% or at least 99% identity thereto. In some embodiments, the non-target antigen comprises HLA-B*07 and the second inhibitory receptor comprises a sequence of SEQ ID NO: 21970. The corresponding nucleotide sequence comprises SEQ ID NO: 21971.

In some embodiments, the second, inhibitory antigen is HLA-A*11, and the inhibitory antigen binding domain comprises an HLA-A*11 antigen binding domain. In another illustrative embodiment, the non-target antigen comprises HLA-A*11, and the second inhibitory receptor comprises a sequence of SEQ ID NO: 21968, or a sequence having at least 90%, at least 95%, at least 97% or at least 99% identity thereto. In some embodiments, the non-target antigen comprises HLA-A*11 and the second inhibitory receptor comprises a sequence of SEQ ID NO: 21968. The corresponding nucleotide sequence comprises SEQ ID NO: 21969.

In some embodiments, the HLA-A*11 antigen binding domain comprises a heavy chain (HC) comprising more CDRs selected from the group consisting of SEQ ID NOS: 10-28.

In some embodiments, the antigen binding domain comprises the antigen binding domain comprises (a) a heavy chain (HC) complementarity determining region 1 (CDR1) sequence selected from the group consisting of SGGYYWS (SEQ ID NO: 10), TSGVGVG (SEQ ID NO: 11), SYAMH (SEQ ID NO: 12), SYDMH (SEQ ID NO: 13), and SYWMH (SEQ ID NO: 14); (b) a HC CDR2 sequence selected from the group consisting of YIYYSGSTYYNPSLKS (SEQ ID NO: 15), LIYWNDDKRYSPSLKS (SEQ ID NO: 16), WINAGNGNTKYSQKFQG (SEQ ID NO: 17), AIGTAGDTYYPGSVKG (SEQ ID NO: 18), and RINSDGSSTSYADSVKG (SEQ ID NO: 19); and (c) a HC CDR3 sequence selected from the group consisting of HYYYYSMDV (SEQ ID NO: 20), HYYYYYLDV (SEQ ID NO: 21), HYYYYMDV (SEQ ID NO: 22), HYYYYYMDV (SEQ ID NO: 23), KTTSFYFDY (SEQ ID NO: 24), RHMRLSCFDY (SEQ ID NO: 25), EGNGANPDAFDI (SEQ ID NO: 26), DLPGSYWYFDL (SEQ ID NO: 27), and GVLLYNWFDP (SEQ ID NO: 28).

In some embodiments, the HLA-A*11 antigen binding domain comprises a heavy chain. In some embodiments the HC comprises a CDR1 sequence of SGGYYWS (SEQ ID NO: 10), a CDR2 sequence of YIYYSGSTYYNPSLKS (SEQ ID NO: 15), and a CDR3 sequence of HYYYYYMDV (SEQ ID NO: 23). In some embodiments, the HC comprises a CDR1 sequence of TSGVGVG (SEQ ID NO: 11), a CDR2 sequence of LIYWNDDKRYSPSLKS (SEQ ID NO: 16), and a CDR3 sequence of KTTSFYFDY (SEQ ID NO: 24). In some embodiments, the HC comprises a CDR1 sequence of SGGYYWS (SEQ ID NO: 10), a CDR2 sequence of YIYYSGSTYYNPSLKS (SEQ ID NO: 15), and a CDR3 sequence of HYYYYMDV (SEQ ID NO: 22). In some embodiments, the HC comprises a CDR1 sequence of SYWMH (SEQ ID NO: 14), a CDR2 sequence of RINSDGSSTSYADSVKG (SEQ ID NO: 19), and a CDR3 sequence of GVLLYNWFDP (SEQ ID NO: 28). In some embodiments, the HC comprises a CDR1 sequence of SGGYYWS (SEQ ID NO: 10), a CDR2 sequence of YIYYSGSTYYNPSLKS (SEQ ID NO: 15) and a CDR3 sequence of HYYYYYLDV (SEQ ID NO: 21). In some embodiments, the HC comprises a CDR1 sequence of SYDMH (SEQ ID NO: 13), a CDR2 sequence of AIGTAGDTYYPGSVKG (SEQ ID NO: 18), and a CDR3 sequence of HYYYYYLDV (SEQ ID NO: 21). In some embodiments, the HC comprises a CDR1 sequence of SYDMH (SEQ ID NO: 13), a CDR2 sequence of AIGTAGDTYYPGSVKG (SEQ ID NO: 18), and a CDR3 sequence of DLPGSYWYFDL (SEQ ID NO: 27). In some embodiments, the HC comprises a CDR1 sequence of SYAMH (SEQ ID NO: 12), a CDR2 sequence of WINAGNGNTKYSQKFQG (SEQ ID NO: 17) and a CDR3 sequence of EGNGANPDAFDI (SEQ ID NO: 26). In some embodiments, the HC comprises a CDR1 sequence of TSGVGVG (SEQ ID NO: 11), a CDR2 sequence of LIYWNDDKRYSPSLKS (SEQ ID NO: 16), and a CDR3 sequence of RHMRLSCFDY (SEQ ID NO: 25). In some embodiments, the HC comprises a CDR1 sequence of SGGYYWS (SEQ ID NO: 10), a CDR2 sequence of YIYYSGSTYYNPSLKS (SEQ ID NO: 15), and a CDR3 sequence of HYYYYSMDV (SEQ ID NO: 20). In some embodiments, the HLA-A*11 antigen binding domain further comprises a light chain.

In some embodiments, the HLA-A*11 antigen binding domain comprises a light chain. In some embodiments the LC comprises a light chain (LC) complementarity determining region 1 (CDR1) comprising a sequence of RASQSISSYLN (SEQ ID NO: 29), a LC CDR2 comprising a sequence of AASSLQS (SEQ ID NO: 30) and a LC CDR3 comprising a sequence of QQSYSTPLT (SEQ ID NO: 31).

In some embodiments, the HLA-A*11 antigen binding domain comprises a HC CDR1 comprising TSGVGVG (SEQ ID NO: 11), a HC CDR2 comprising LIYWNDDKRYSPSLKS (SEQ ID NO: 16), a HC CDR3 comprising KTTSFYFDY (SEQ ID NO: 24), a LC CDR1 comprising RASQSISSYLN (SEQ ID NO: 29), a LC CDR2 comprising AASSLQS (SEQ ID NO: 30), and a LC CDR3 comprising QQSYSTPLT (SEQ ID NO: 31).

In some embodiments, the HLA-A*11 antigen binding domain comprises a HC CDR1 comprising SYWMH (SEQ ID NO: 14), a HC CDR2 comprising RINSDGSSTSYADSVKG (SEQ ID NO: 19), a HC CDR comprising GVLLYNWFDP (SEQ ID NO: 28), a LC CDR1 comprising RASQSISSYLN (SEQ ID NO: 29), a LC CDR2 comprising AASSLQS (SEQ ID NO: 30), and a LC CDR3 comprising QQSYSTPLT (SEQ ID NO: 31).

In some embodiments, the non-target antigen comprises HLA-A. In some embodiments, the antigen binding domain of the second, inhibitory receptor comprises an HLA-A*01, HLA-A*02, HLA-A*03 or HLA-A*11 antigen binding domain comprising CDR sequences as set forth in Table 5.

In some embodiments, the non-target antigen comprises HLA-B. In some embodiments, the antigen binding domain of the second, inhibitory receptors comprises an HLA-B*07 antigen binding domain comprising CDR sequences as set forth in Table 5.

In some embodiments, the non-target antigen comprises HLA-C. In some embodiments, the antigen binding domain of the second, inhibitory receptors comprises an HLA-C*07antigen binding domain comprising CDR sequences as set forth in Table 5.

In some embodiments, the extracellular antigen binding domain of the second receptor specifically binds an allelic variant of an HLA-A, HLA-B, or HLA-C protein. In some embodiments, the extracellular antigen binding domain of the second receptor specifically binds to HLA-A*01, HLA-A*02, HLA-A*03, HLA-A*11, HLA-B*07, or HLA-C*07.

In some embodiments, the extracellular antigen binding domain of the second receptor specifically binds to HLA-A*01. In some embodiments, the extracellular antigen binding domain of the second receptor comprises HLA-A*01 complementarity determining regions (CDRs) CDR-L1, CDR-L2, CDR-L3, CDR-H1, CDR-H2, CDR-H3 as disclosed Table 5; or CDR sequences having at most 1, 2, or 3 substitutions, deletions, or insertions relative to the HLA-A*01 CDRs of Table 5.

In some embodiments, the extracellular antigen binding domain of the second receptor specifically binds to HLA-A*02. In some embodiments, the extracellular antigen binding domain of the second receptor comprises HLA-A*02 complementarity determining regions (CDRs) CDR-L1, CDR-L2, CDR-L3, CDR-H1, CDR-H2, CDR-H3 as disclosed Table 5; or CDR sequences having at most 1, 2, or 3 substitutions, deletions, or insertions relative to the HLA-A*02 CDRs of Table 5.

In some embodiments, the non-target antigen comprises HLA-A*03, and the ligand binding domain of the second receptor comprises an HLA-A*03 ligand binding domain. In some embodiments, the ligand binding domain binds HLA-A*03 independent of the peptide in a pMHC complex comprising HLA-A*03. In some embodiments, the HLA-A*03 ligand binding domain comprises an scFv domain. In some embodiments, the HLA-A*03 ligand binding domain comprises a sequence of any one of SEQ ID NOs: 351-364 or SEQ ID NO: 21956. In some embodiments, the HLA-A*03 ligand binding domain comprises a sequence at least 90%, at least 95%, at least 97% or at least 99% identical to a sequence of any one of SEQ ID NOs: 351-364 or SEQ ID NO: 21956.

In some embodiments, the non-target antigen comprises HLA-A*03, and the extracellular ligand binding domain of the second receptor comprises a sequence of SEQ ID NOs: 21956, or a sequence having at least 90%, at least 95%, at least 97%, or at least 99% identity thereto.

In some embodiments, the non-target antigen comprises HLA-A*03, and the extracellular ligand binding domain of the second receptor comprises a VL comprising a sequence of DIVMTQSHKFMSTSVGDRVSITCKASQDVSTTVAWYQQKPGQSPKLLIYSASYRY TGVPDRFTGSGSGTDFTFTISSVQAEDLAVYYCQQHYSTPPTFGGGTKLEIK (SEQ ID NO: 21963), or a sequence having at least 90%, at least 95%, at least 97%, or at least 99% identity thereto. In some embodiments, the extracellular ligand binding domain of the second receptor comprises a VH comprising a sequence of EVKLEESGGGLVQPGGSMKLSCVASGFTFSNYWMNWVRQSPEKGLEWVAEIRL KSTNYATHYAESVKGRFTISRDDSKSSVYLQMNNLRAEDTGIYYCTTLITPDYWG QGTTLTVSS (SEQ ID NO: 21964), or a sequence having at least 90%, at least 95%, at least 97%, or at least 99% identity thereto. In some embodiments, the VH and VL are separated by a linker, for example GGGGSGGGGSGGGGSGG (SEQ ID NO: 491). In some embodiments, the VH and VL are ordered, from N to C terminal, VH, linker and VL. In some embodiments, the VH and VL are ordered, from N to C terminal, VL, linker and VH.

In some embodiments, the HLA-A*03 extracellular ligand binding domain comprises the complementarity determined regions (CDRs) of any one of SEQ ID NOS: 10, 15, 374-408, 21957-21962, or 21972. In some embodiments, the extracellular ligand binding domain comprises a sequence at least 95% identical to any one of SEQ ID NOS: 10, 15, 374-408, 21957-21962, or 21972. In some embodiments, the HLA-A*03 antigen binding domain comprises a heavy chain and a light chain, and the heavy chain comprises CDRs selected from SEQ ID NOs: 10, 15, 374-387, 391-397, 104, 405-408, 21960-21962, and 21972 and the light chain comprises CDRs selected from SEQ ID NOs: 29-31, 388-390, 398-400, 402-404, and 21957-21959. In some embodiments, the extracellular ligand binding domain of the second receptor comprises complementarity determining regions (CDRs) CDR-L1, CDR-L2, CDR-L3, CDR-H1, CDR-H2, CDR-H3 of Table 5.

In some embodiments, the HLA-A*03 antigen binding domain comprises a heavy chain and a light chain, and the heavy chain comprises a sequence at least 95% identical to the heavy chain portion of any one of SEQ ID NOS: 351-364 or SEQ ID NO: 21956, and the light chain comprises a sequence at least 95% identical to the light chain portion of any one of SEQ ID NOS: 351-364 or SEQ ID NO: 21956.

In further embodiments of any of the antigen binding domains, each CDR sequence may have 1, 2, 3 or more substitutions, insertions, or deletions. CDR sequences may tolerate substitutions, deletions, or insertions. Using sequence alignment tools, routine experimentation, and known assays, those of skill in the art may generate and test variant sequences having 1, 2, 3, or more substitutions, insertions, or deletions in CDR sequences without undue experimentation.

In some embodiments, the HLA-A*11 antigen binding domain comprises a heavy chain and a light chain.

In some embodiments, the antigen binding domain comprises a variable heavy chain comprising a sequence of SEQ ID NOS: 32-40, or a sequence having at least 90%, at least 95%, at least 97%, at least 98% or at least 99% identity thereto. In some embodiments, the antigen binding domain comprises a variable heavy chain comprising a sequence of SEQ ID NO: 37, or a sequence having at least 90%, at least 95%, at least 97%, at least 98% or at least 99% identity thereto. In some embodiments, the antigen binding domain comprises a variable heavy chain comprising a sequence of SEQ ID NO: 39, or a sequence having at least 90%, at least 95%, at least 97%, at least 98% or at least 99% identity thereto. In some embodiments, the antigen binding domain comprises a variable heavy chain comprising a sequence of SEQ ID NOS: 32-40. In some embodiments, the antigen binding domain comprises a variable heavy chain comprising a sequence of SEQ ID NO: 37. In some embodiments, the antigen binding domain comprises a variable heavy chain comprising a sequence of SEQ ID NO: 39.

In some embodiments, the antigen binding domain comprises a variable light chain comprising a sequence of SEQ ID NO: 41, or a sequence having at least 90%, at least 95%, at least 97%, at least 98% or at least 99% identity thereto. In some embodiments, the antigen binding domain comprises a variable light chain comprising a sequence of SEQ ID NO: 41.

In some embodiments, the antigen binding domain comprises a variable heavy chain and a variable light chain. In some embodiments, the variable heavy chain comprises a sequence of SEQ ID NOS: 32-40, and the variable light chain comprises a sequence of SEQ ID NO: 41. In some embodiments, the variable heavy chain comprises a sequence of SEQ ID NO: 37, and the variable light chain comprises a sequence of SEQ ID NO: 41. In some embodiments, the variable heavy chain comprises a sequence of SEQ ID NO: 39, and the variable light chain comprises a sequence of SEQ ID NO: 41.

TABLE 6
Exemplary heavy and light chain sequences for HLA-A*11 antigen binding
domains

| Heavy Chain | | |
|---|---|---|
| Sequences | Protein Sequence | DNA Sequence |
| 9 | QVQLQESGPGLVKPSQTLSLTCTVSGGSISSGGYY | (SEQ ID NO: |
| | WSWIRQPPGKGLEWIGYIYYSGSTYYNPSLKSRV | 42) |
| | TISVDTSKNQFSLKLSSVTAADTAVYYCARHYYY | |
| | YSMDVWGKGTTVTVSS (SEQ ID NO: 32) | |
| 8 | QITLKESGPTLVKPTQTLTLTCTFSGFSLSTSGVGV | (SEQ ID NO: |
| | GWIRQPPGKALEWLALIYWNDDKRYSPSLKSRLT | 43) |
| | ITKDTSKNQVVLTMTNMDPVDTATYYCAHRHMR | |
| | LSCFDYWGQGTLVTVSS (SEQ ID NO: 33) | |
| 7 | QVQLVQSGAEVKKPGASVKVSCKASGYTFTSYA | (SEQ ID NO: |
| | MHWVRQAPGQRLEWMGWINAGNGNTKYSQKF | 44 |
| | QGRVTITRDTSASTAYMELSSLRSEDTAVYYCAR | |
| | EGNGANPDAFDIWGQGTMVTVSS (SEQ ID NO: 34) | |
| 6 | EVQLVESGGGLVQPGGSLRLSCAASGFTFSSYDM | (SEQ ID NO: |
| | HWVRQATGKGLEWVSAIGTAGDTYYPGSVKGRF | 45) |
| | TISRENAKNSLYLQMNSLRAGDTAVYYCARDLPG | |
| | SYWYFDLWGRGTLVTVSS (SEQ ID NO: 35) | |
| 5 | QVQLQESGPGLVKPSQTLSLTCTVSGGSISSGGYY | (SEQ ID NO: |
| | WSWIRQPPGKGLEWIGYIYYSGSTYYNPSLKSRV | 46) |
| | TISVDTSKNQFSLKLSSVTAADTAVYYCARHYYY | |
| | YYLDVWGKGTTVTVSS (SEQ ID NO: 36) | |
| 4 | EVQLVESGGGLVQPGGSLRLSCAASGFTFSSYWM | (SEQ ID NO: |
| | HWVRQAPGKGLVWVSRINSDGSSTSYADSVKGR | 47) |
| | FTISRDNAKNTLYLQMNSLRAEDTAVYYCCLGVL | |
| | LYNWFDPWGQGTLVTVSS (SEQ ID NO: 37) | |
| 3 | QVQLQESGPGLVKPSQTLSLTCTVSGGSISSGGYY | (SEQ ID NO: |
| | WSWIRQPPGKGLEWIGYIYYSGSTYYNPSLKSRV | 48 |
| | TISVDTSKNQFSLKLSSVTAADTAVYYCARHYYY | |
| | YMDVWGKGTTVTVSS (SEQ ID NO: 38) | |
| 2 | QITLKESGPTLVKPTQTLTLTCTFSGFSLSTSGVGV | (SEQ ID NO: |
| | GWIRQPPGKALEWLALIYWNDDKRYSPSLKSRLT | 49 |
| | ITKDTSKNQVVLTMTNMDPVDTATYYCAHKTTS | |
| | FYFDYWGQGTLVTVSS (SEQ ID NO: 39) | |
| 1 | QVQLQESGPGLVKPSQTLSLTCTVSGGSISSGGYY | (SEQ ID NO: |
| | WSWIRQPPGKGLEWIGYIYYSGSTYYNPSLKSRV | 50) |
| | TISVDTSKNQFSLKLSSVTAADTAVYYCARHYYY | |
| | YYMDVWGKGTTVTVSS (SEQ ID NO: 40) | |
| Light Chain | DIQMTQSPSSLSASVGDRVTITCRASQSISSYLNW | (SEQ ID NO: |
| Sequence | YQQKPGKAPKLLIYAASSLQSGVPSRFSGSGSGTD | 51) |
| | FTLTISSLQPEDFATYYCQQSYSTPLTFGGGTKVEI | |
| | K (SEQ ID NO: 41) | |

In some embodiments, provided herein is an antigen binding domain that specifically binds to MHC I comprising an α11 chain encoded by an HLA-A*11 allele, as described herein. In some embodiments, the HLA-A*11 antigen binding domain is a scFv. The scFv can be in either orientation, i.e. a variable heavy chain (VH)-linker-variable light chain (VL) or a VL-linker-VH orientation.

In some embodiments, the variable heavy chain of the scFv comprises a sequence selected from the group consisting of SEQ ID NOS: 32-40, or a sequence having at least 90%, at least 95%, at least 97%, at least 98% or at least 99% identity thereto, and the variable light chain of the scFv, comprises a sequence of SEQ ID NO: 41, or a sequence having at least 90%, at least 95%, at least 97%, at least 98% or at least 99% identity thereto. In some embodiments, the variable heavy chain of the scFv comprises a sequence selected from the group consisting of SEQ ID NOS: 32-40, and the variable light chain of the scFv comprises a sequence of SEQ ID NO: 41. In some embodiments, the variable heavy chain of the scFv comprises a sequence selected from the group consisting of SEQ ID NOS: 37 and 39, and the variable light chain of the scFv comprises a sequence of SEQ ID NO: 41.

In some embodiments, the scFv comprises a sequence selected from the group set forth in Tables 3 and 4, supra. In some embodiments, the scFv comprises a sequence selected from the group consisting of SEQ ID NOS: 52-60, or a sequence having at least 90%, at least 95%, at least 97%, at least 98% or at least 99% identity thereto. In some embodiments, the scFv comprises a sequence selected from the group consisting of SEQ ID NOS: 52-60. In some embodiments, the scFv comprises a sequence of SEQ ID NO:57 or SEQ ID NO: 59, or a sequence having at least 90%, at least 95%, at least 97%, at least 98% or at least 99% identity thereto. In some embodiments, the scFv comprises a sequence of SEQ ID NO: 57 or SEQ ID NO: 59.

### Minor Histocompatibility Antigens

In some embodiments, the second, inhibitor antigen comprises a minor histocompatibility antigen (MiHA). In some embodiments, the second, inhibitor antigen comprises an allele of a MiHA that is lost in a target cell through LOH.

MiHAs are peptides derived from proteins that contain nonsynonymous differences between alleles and are displayed by common HLA alleles. The non-synonymous differences can arise from SNPs, deletions, frameshift mutations or insertions in the coding sequence of the gene encoding the MiHA. Exemplary MiHAs can be about 9-12 amino acids in length and can bind to MHC class I and MHC class II proteins. Binding of the TCR to the MHC complex displaying the MiHA can activate T cells. The genetic and immunological properties of MiHAs will be known to the person of ordinary skill in the art. Candidate MiHAs are known peptides presented by known HLA class I alleles, are known to elicit T cell responses in the clinic (for example, in graft versus host disease, or transplant rejection, and allow for patient selection by simple SNP genotyping.

In some embodiments, the MiHA has broad or ubiquitous RNA expression.

In some embodiments, the MiHA has high minor allele frequency.

In some embodiments, the MiHA comprises a peptide derived from a Y chromosome gene.

In some embodiments, the second, inhibitory antigen comprises HA-1(H). In some embodiments, the second, inhibitory antigen binding is isolated or derived from a TCR. In some embodiments, the second, inhibitory antigen binding domain comprises TCR alpha and TCR beta variable domains. In some embodiments, the TCR alpha and TCR beta variable domains are separated by a self-cleaving polypeptide sequence. In some embodiments, the TCR alpha and TCR beta variable domains separated by a self-cleaving polypeptide sequence comprise SEQ ID NO: 196. In some embodiments, the TCR alpha and TCR beta variable domains separated by a self-cleaving polypeptide sequence comprise SEQ ID NO: 196, or a sequence having at least 90%, at least 95%, or at least 99% identity thereto. In some embodiments, the TCR alpha and TCR beta variable domains are encoded by a sequence of SEQ ID NO: 202, or a sequence having at least 80% identity, at least 90%, at least 95%, or at least 99% identity thereto. In some embodiments, the TCR alpha variable domain comprises SEQ ID NO: 198 or a sequence having at least 90%, at least 95%, or at least 99% identity thereto. In some embodiments, the TCR beta variable domain comprises SEQ ID NO: 199 or a sequence having at least 90%, at least 95%, or at least 99% identity thereto.

Illustrative antigen binding domains that selectively bind to HA-1 variant H peptide (VLHDDLLEA (SEQ ID NO: 195)) are provided in SEQ ID NOs: 202-206. TCR alpha and TCR beta sequences are separated by a P2A self-cleaving polypeptide of sequence ATNFSLLKQAGDVEENPGP (SEQ ID NO: 196) with an N terminal GSG linker.

### Loss of Y Chromosome Antigens

In some embodiments, the second, inhibitor antigen comprises a Y chromosome gene, i.e. peptide encoded by a gene on the Y chromosome. In some embodiments, the second, inhibitor antigen comprises a peptide encoded by a Y chromosome gene that is lost in target cells through loss of Y chromosome (LoY). For example, about a third of the characterized MiHAs come from the Y chromosome. The Y chromosome contains over 200 protein coding genes, all of which are envisaged as within the scope of the instant disclosure.

As used herein, “loss of Y”, or “LoY” refers a genetic change that occurs at high frequency in tumors whereby one copy of part or all of the Y chromosome is deleted, leading to a loss of Y chromosome encoded gene(s).

Loss of Y chromosome is known to occur in certain cancers. For example, there is a reported 40% somatic loss of Y chromosome in renal clear cell cancers (Arseneault et al., Sci. Rep. 7: 44876 (2017)). Similarly, clonal loss of the Y chromosome was reported in 5 out of 31 in male breast cancer subjects (Wong et al., Oncotarget 6(42):44927-40 (2015)). Loss of the Y chromosome in tumors from male patients has been described as a “consistent feature” of head and neck cancer patients (el-Naggar et al., Am J Clin Pathol 105(1):102-8 (1996)). Further, Y chromosome loss was associated with X chromosome disomy in four of seven male patients with gastric cancer (Saal et al., Virchows Arch B Cell Pathol (1993)). Thus, Y chromosome genes can be lost in a variety of cancers, and can be used as inhibitor antigens with the engineered receptors of the instant disclosure targeting cancer cells.

### Components of Activator Receptors

In some embodiments, the either the first, activator receptor is a chimeric antigen receptor (CAR). In some embodiments, the first and second engineered receptors are chimeric antigen receptors. All CAR architectures are envisaged as within the scope of the instant disclosure. In some embodiments, the first, activator receptor is a T cell receptor (TCR).

The term “chimeric antigen receptors (CARs)” as used herein, may refer to artificial receptors derived from T-cell receptors and encompasses engineered receptors that graft an artificial specificity onto a particular immune effector cell. CARs may be employed to impart the specificity of a monoclonal antibody onto a T cell, thereby allowing a large number of specific T cells to be generated, for example, for use in adoptive cell therapy. In specific embodiments, CARs direct specificity of the cell to a tumor associated antigen, for example. Exemplary CARs comprise an intracellular activation domain, a transmembrane domain, and an extracellular domain comprising a tumor associated antigen binding region. In some embodiments, CARs further comprise a hinge domain. In particular aspects, CARs comprise fusions of single-chain variable fragments (scFv) derived from monoclonal antibodies, fused to a CD3 transmembrane domain and endodomain. The specificity of other CAR designs may be derived from ligands of receptors (e.g., peptides). In certain cases, CARs comprise domains for additional co-stimulatory signaling, such as CD3, 4-1BB, FcR, CD27, CD28, CD137, DAP10, and/or OX40. In some cases, molecules can be co-expressed with the CAR, including co-stimulatory molecules, reporter genes for imaging, gene products that conditionally ablate the T cells upon addition of a pro-drug, homing receptors, cytokines, and cytokine receptors.

In some embodiments, the extracellular antigen binding domain of the first receptor is fused to the extracellular domain of a CAR.

### Extracellular Domains

In some embodiments, the extracellular domain of the CAR comprises an antigen binding domains described supra.

### Hinge Region

In some embodiments, the CARs of the present disclosure comprise a hinge region. Incorporation of a hinge region can affect cytokine production from CAR-T cells and improve expansion of CAR-T cells in vivo. Exemplary hinges can be isolated or derived from IgG, CD28 or CD8, among others, for example IgG1.

In some embodiments, the hinge is isolated or derived from CD8a or CD28. In some embodiments, the CD8a hinge comprises an amino acid sequence having at least 80% identity, at least 90% identity, at least 95% identity, at least 99% identity or is identical to a sequence of TTTPAPRPPTPAPTIASQPLSLRPEACRPAAGGAVHTRGLDFACD (SEQ ID NO: 219). In some embodiments, the CD8a hinge comprises SEQ ID NO: 219. In some embodiments, the CD8a hinge consists essentially of SEQ ID NO: 219. In some embodiments, the CD8a hinge is encoded by a nucleotide sequence having at least 80% identity, at least 90% identity, at least 95% identity, at least 99% identity or is identical to a sequence of ACCACGACGCCAGCGCCGCGACCACCAACACCGGCGCCCACCATCGCGTCGC AGCCCCTGTCCCTGCGCCCAGAGGCGTGCCGGCCAGCGGCGGGGGGCGCAGT GCACACGAGGGGGCTGGACTTCGCCTGTGAT (SEQ ID NO: 220). In some embodiments, the CD8a hinge is encoded by SEQ ID NO: 220.

In some embodiments, the CD28 hinge comprises an amino acid sequence having at least 80% identity, at least 90% identity, at least 95% identity, at least 99% identity or is identical to a sequence of CTIEVMYPPPYLDNEKSNGTIIHVKGKHLCPSPLFPGPSKP (SEQ ID NO: 221). In some embodiments, the CD28 hinge comprises or consists essentially of SEQ ID NO: 221. In some embodiments, the CD28 hinge is encoded by a nucleotide sequence having at least 80% identity, at least 90% identity, at least 95% identity, at least 99% identity or is identical to a sequence of:

(SEQ ID NO: 222)
TGTACCATTGAAGTTATGTATCCTCCTCCTTACCTAGACAATGAGAAGA
GCAATGGAACCATTATCCATGTGAAAGGGAAACACCTTTGTCCAAGTCC
CCTATTTCCCGGACCTTCTAAGCCC.

In some embodiments, the CD28 hinge is encoded by SEQ ID NO: 222.

In some embodiments, the activator receptor comprises a hinge sequence isolated or derived from CD8, CD28, IgG1, or IgG4, or a synthetic hinge.

### Transmembrane Domain

The CARs of the present disclosure can be designed to comprise a transmembrane domain that is fused to the extracellular domain or hinge domain of the CAR. In some embodiments, the transmembrane domain that naturally is associated with one of the domains in the CAR is used. For example, a CAR comprising a CD28 co-stimulatory domain might also use a CD28 transmembrane domain. In some instances, the transmembrane domain can be selected or modified by amino acid substitution to avoid binding of such domains to the transmembrane domains of the same or different surface membrane proteins to minimize interactions with other members of the receptor complex.

The transmembrane domain may be derived either from a natural or from a synthetic source. Where the source is natural, the domain may be derived from any membrane-bound or transmembrane protein. Transmembrane regions may be isolated or derived from (i.e. comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154, or from an immunoglobulin such as IgG4. Alternatively the transmembrane domain may be synthetic, in which case it will comprise predominantly hydrophobic residues such as leucine and valine. In some embodiments, a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the CAR. A glycine-serine doublet provides a particularly suitable linker.

In some embodiments of the CARs of the disclosure, the CARs comprise a CD28 transmembrane domain. In some embodiments, the CD28 transmembrane domain comprises an amino acid sequence having at least 80% identity, at least 90% identity, at least 95% identity, at least 99% identity or is identical to a sequence of FWVLVVVGGVLACYSLLVTVAFIIFWV (SEQ ID NO: 232). In some embodiments, the CD28 transmembrane domain comprises or consists essentially of SEQ ID NO: 232. In some embodiments, the CD28 transmembrane domain is encoded by a nucleotide sequence having at least 80% identity, at least 90% identity, at least 95% identity, at least 99% identity or is identical to a sequence of

(SEQ ID NO: 233)
TTCTGGGTGCTGGTCGTTGTGGGCGGCGTGCTGGCCTGCTACAGCCTGC
TGGTGACAGTGGCCTTCATCATCTTTTGGGTG.

In some embodiments, the CD28 transmembrane domain is encoded by SEQ ID NO: 233. In some embodiments, the CD28 transmembrane domain is encoded by a nucleotide sequence having at least 80% identity, at least 90% identity, at least 95% identity, at least 99% identity or is identical to a sequence of TTCTGGGTGCTGGTCGTTGTGGGCGGCGTGCTGGCCTGCTACAGCCTGCTGGT GACAGTGGCCTTCATCATCTTTTGGGTG (SEQ ID NO: 486). In some embodiments, the CD28 transmembrane domain is encoded by SEQ ID NO: 486.

In some embodiments of the CARs of the disclosure, the CARs comprise an IL-2Rbeta transmembrane domain. In some embodiments, the IL-2Rbeta transmembrane domain comprises an amino acid sequence having at least 80% identity, at least 90% identity, at least 95% identity, at least 99% identity or is identical to a sequence of IPWLGHLLVGLSGAFGFIILVYLLI (SEQ ID NO: 234). In some embodiments, the IL-2Rbeta transmembrane domain comprises or consists essentially of SEQ ID NO: 234. In some embodiments, the IL-2Rbeta transmembrane domain is encoded by a nucleotide sequence having at least 80% identity, at least 90% identity, at least 95% identity, at least 99% identity or is identical to a sequence of

(SEQ ID NO: 235)
ATTCCGTGGCTCGGCCACCTCCTCGTGGGCCTCAGCGGGGCTTTTGGCT
TCATCATCTTAGTGTACTTGCTGATC.

In some embodiments, the IL-2Rbeta transmembrane domain is encoded by SEQ ID NO: 235.

In some embodiments, the CAR comprises a transmembrane domain isolated or derived from CD8. In some embodiments, the CD8 transmembrane domain comprises an amino acid sequence having at least 80% identity, at least 90% identity, at least 95% identity, at least 99% identity or is identical to a sequence of IYIWAPLAGTCGVLLLSLVIT (SEQ ID NO: 236). In some embodiments, the CD8 transmembrane domain comprises a sequence of SEQ ID NO: 236. In some embodiments, the CD8 transmembrane domain is encoded by a nucleotide sequence having at least 80% identity, at least 90% identity, at least 95% identity, at least 99% identity or is identical to a sequence of

(SEQ ID NO: 237)
ATCTACATCTGGGCGCCCTTGGCCGGGACTTGTGGGGTCCTTCTCCTGT
CACTGGTTATCACC.

### Cytoplasmic Domain

The disclosure provides an activator receptor comprising an extracellular antigen binding domain specific to an HLA-E antigen. In some embodiments, the activator receptor is a T cell receptor (TCR) or a chimeric antigen receptor (CAR). In some embodiments, the CAR comprises an intracellular domain isolated or derived from CD28, 4-1BB or CD3z, or a combination thereof.

The cytoplasmic domain or otherwise the intracellular signaling domain of the CARs of the instant invention is responsible for activation of at least one of the normal effector functions of the immune cell in which the CAR has been placed. The term “effector function” refers to a specialized function of a cell. Effector functions of a regulatory T cell, for example, include the suppression or downregulation of induction or proliferation of effector T cells. Thus the term “intracellular signaling domain” refers to the portion of a protein which transduces the effector function signal and directs the cell to perform a specialized function. While usually the entire intracellular signaling domain can be employed, in many cases it is not necessary to use the entire domain. To the extent that a truncated portion of the intracellular signaling domain is used, such truncated portion may be used in place of the intact chain as long as it transduces the effector function signal. In some cases, multiple intracellular domains can be combined to achieve the desired functions of the CAR-T cells of the instant disclosure. The term intracellular signaling domain is thus meant to include any truncated portion of one or more intracellular signaling domains sufficient to transduce the effector function signal.

Examples of intracellular signaling domains for use in the CARs of the instant disclosure include the cytoplasmic sequences of the T cell receptor (TCR) and co-receptors that act in concert to initiate signal transduction following antigen receptor engagement, as well as any derivative or variant of these sequences and any synthetic sequence that has the same functional capability.

Accordingly, the intracellular domain of CARs of the instant disclosure comprises at least one cytoplasmic activation domain. In some embodiments, the intracellular activation domain ensures that there is T-cell receptor (TCR) signaling necessary to activate the effector functions of the CAR T-cell. In some embodiments, the at least one cytoplasmic activation is a CD247 molecule (CD3ζ) activation domain, a stimulatory killer immunoglobulin-like receptor (KIR) KIR2DS2 activation domain, or a DNAX-activating protein of 12 kDa (DAP12) activation domain. In some embodiments, the CD3ζ activation domain comprises an amino acid sequence having at least 80% identity, at least 90% identity, at least 95% identity, at least 99% identity or is identical to a sequence of

(SEQ ID NO: 238)
RVKFSRSADAPAYKQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR.

In some embodiments, the CD3ζ activation domain comprises or consists essentially of SEQ ID NO: 238. In some embodiments, the CD3ζ activation domain is encoded by a nucleotide sequence having at least 80% identity, at least 90% identity, at least 95% identity, at least 99% identity or is identical to a sequence of AGAGTGAAGTTCAGCAGGAGCGCAGACGCCCCCGCGTACAAGCAGGGCCAGA ACCAGCTCTATAACGAGCTCAATCTAGGACGAAGAGAGGAGTACGATGTTTTG GACAAGCGTAGAGGCCGGGACCCTGAGATGGGGGGAAAGCCGAGAAGGAAG AACCCTCAGGAAGGCCTGTACAATGAACTGCAGAAAGATAAGATGGCGGAGG CCTACAGTGAGATTGGGATGAAAGGCGAGCGCCGGAGGGGCAAGGGGCACG ATGGCCTTTACCAGGGACTCAGTACAGCCACCAAGGACACCTACGACGCCCTT CACATGCAGGCCCTGCCCCCTCGC (SEQ ID NO: 239). In some embodiments, the CD3ζ activation domain is encoded by SEQ ID NO: 239.

In some embodiments, the CD3ζ activation domain is encoded by a nucleotide sequence having at least 80% identity, at least 90% identity, at least 95% identity, at least 99% identity or is identical to a sequence of SEQ ID NO: 21982. In some embodiments, the CD3ζ activation domain is encoded by SEQ ID NO: 21982.

It is known that signals generated through the TCR alone are often insufficient for full activation of the T cell and that a secondary or co-stimulatory signal is also required. Thus, T cell activation can be said to be mediated by two distinct classes of cytoplasmic signaling sequence: those that initiate antigen-dependent primary activation through the TCR (primary cytoplasmic signaling sequences) and those that act in an antigen-independent manner to provide a secondary or co-stimulatory signal (secondary cytoplasmic signaling sequences).

Primary cytoplasmic signaling sequences regulate primary activation of the TCR complex either in a stimulatory way, or in an inhibitory way. Primary cytoplasmic signaling sequences that act in a stimulatory manner may contain signaling motifs which are known as immunoreceptor tyrosine-based activation motifs or ITAMs. In some embodiments, the ITAM contains a tyrosine separated from a leucine or an isoleucine by any two other amino acids (YxxL) (SEQ ID NO: 240).

In some embodiments, the cytoplasmic domain contains 1, 2, or 3 ITAMs. In some embodiments, the cytoplasmic domain contains 1 ITAM. In some embodiments, the cytoplasmic domain contains 2 ITAMs. In some embodiments, the cytoplasmic domain contains 3 ITAMs. In some embodiments, the cytoplasmic domain contains 4 ITAMs. In some embodiments, the cytoplasmic domain contains 5 ITAMs.

In some embodiments, the cytoplasmic domain is a CD3ζ activation domain. In some embodiments, CD3ζ activation domain comprises a single ITAM. In some embodiments, CD3ζ activation domain comprises two ITAMs. In some embodiments, CD3ζactivation domain comprises three ITAMs.

In some embodiments, the CD3ζ activation domain comprising a single ITAM comprises an amino acid sequence having at least 80% identity, at least 90% identity, at least 95% identity, at least 99% identity or is identical to a sequence of RVKFSRSADAPAYQQGQNQLYNELNLGRREEYDVLHMQALPPR (SEQ ID NO: 241). In some embodiments, the CD3ζ activation domain comprises SEQ ID NO: 241. In some embodiments, the CD3ζ activation domain comprising a single ITAM consists essentially of an amino acid sequence of SEQ ID NO: 241. In some embodiments, the CD3ζ activation domain comprising a single ITAM is encoded by a nucleotide sequence having at least 80% identity, at least 90% identity, at least 95% identity, at least 99% identity or is identical to a sequence of AGAGTGAAGTTCAGCAGGAGCGCAGACGCCCCCGCGTACCAGCAGGGCCAGA ACCAGCTCTATAACGAGCTCAATCTAGGACGAAGAGAGGAGTACGATGTTTTG CACATGCAGGCCCTGCCCCCTCGC (SEQ ID NO: 242). In some embodiments, the CD3ζ activation domain is encoded by SEQ ID NO: 242.

Further examples of ITAM containing primary cytoplasmic signaling sequences that can be used in the CARs of the instant disclosure include those derived from TCRζ, FcRγ, FcRβ, CD3γ, CD3δ, CD3ε, CD3ζ, CD5, CD22, CD79a, CD79b, and CD66d. It is particularly preferred that cytoplasmic signaling molecule in the CAR of the instant invention comprises a cytoplasmic signaling sequence derived from CD3ζ.

### Co-Stimulatory Domain

In some embodiments, the cytoplasmic domain of the CAR can be designed to comprise the CD3ζ signaling domain by itself or combined with any other desired cytoplasmic domain(s) useful in the context of the CAR of the instant disclosure. For example, the cytoplasmic domain of the CAR can comprise a CD3ζ chain portion and a co-stimulatory domain. The co-stimulatory domain refers to a portion of the CAR comprising the intracellular domain of a costimulatory molecule. A costimulatory molecule is a cell surface molecule other than an antigen receptor or its antigens that is required for an efficient response of lymphocytes to an antigen. Examples of such molecules include the co-stimulatory domain selected from the group consisting of IL-2Rβ, Fc Receptor gamma (FcRγ), Fc Receptor beta (FcRβ), CD3g molecule gamma (CD3γ), CD3δ, CD3ε, CD5 molecule (CD5), CD22 molecule (CD22), CD79a molecule (CD79a), CD79b molecule (CD79b), carcinoembryonic antigen related cell adhesion molecule 3 (CD66d), CD27 molecule (CD27), CD28 molecule (CD28), TNF receptor superfamily member 9 (4-1BB), TNF receptor superfamily member 4 (OX40), TNF receptor superfamily member 8 (CD30), CD40 molecule (CD40), programmed cell death 1 (PD-1), inducible T cell costimulatory (ICOS), lymphocyte function-associated antigen-1 (LFA-1), CD2 molecule (CD2), CD7 molecule (CD7), TNF superfamily member 14 (LIGHT), killer cell lectin like receptor C2 (NKG2C) and CD276 molecule (B7-H3) c-stimulatory domains, or functional fragments thereof. In some embodiments, the intracellular domains of CARs of the instant disclosure comprise at least one co-stimulatory domain. In some embodiments, the co-stimulatory domain is isolated or derived from CD28.

The cytoplasmic domains within the cytoplasmic signaling portion of the CARs of the instant disclosure may be linked to each other in a random or specified order. Optionally, a short oligo- or polypeptide linker, for example between 2 and 10 amino acids in length may form the linkage. A glycine-serine doublet provides an example of a suitable linker.

In some embodiments, the intracellular domains of CARs of the instant disclosure comprise at least one co-stimulatory domain. In some embodiments, the co-stimulatory domain is isolated or derived from CD28. In some embodiments, the CD28 co-stimulatory domain comprises an amino acid sequence having at least 80% identity, at least 90% identity, at least 95% identity, at least 99% identity or is identical to a sequence of

(SEQ ID NO: 243)
RSKRSRLLHSDYMNMTPRRPGPTRKHYQPYAPPRDFAAYRS.

In some embodiments, the CD28 co-stimulatory domain comprises or consists essentially of SEQ ID NO: 243. In some embodiments, the CD28 co-stimulatory domain is encoded by a nucleotide sequence having at least 80% identity, at least 90% identity, at least 95% identity, at least 99% identity or is identical to a sequence of AGGAGCAAGCGGAGCAGACTGCTGCACAGCGACTACATGAACATGACCCCCC GGAGGCCTGGCCCCACCCGGAAGCACTACCAGCCCTACGCCCCTCCCAGGGAT TTCGCCGCCTACCGGAGC (SEQ ID NO: 244). In some embodiments, the CD28 co-stimulatory domain is encoded by SEQ ID NO: 244.

In some embodiments, the co-stimulatory domain is isolated or derived from 4-1BB. In some embodiments, the 4-1BB co-stimulatory domain comprises an amino acid sequence having at least 80% identity, at least 90% identity, at least 95% identity, at least 99% identity or is identical to a sequence of KRGRKKLLYIFKQPFMRPVQTTQEEDGCSCRFPEEEEGGCEL (SEQ ID NO: 487). In some embodiments, the 4-1BB co-stimulatory domain s encoded by a nucleotide sequence having at least 80% identity, at least 90% identity, at least 95% identity, at least 99% identity or is identical to a sequence of

(SEQ ID NO: 488)
AAACGGGGCAGAAAGAAACTCCTGTATATATTCAAACAACCATTTATGA
GGCCAGTACAAACTACTCAAGAGGAAGATGGCTGTAGCTGCCGATTTCC
AGAAGAAGAAGAAGGAGGATGTGAACTG.

In some embodiments, the intracellular domain of the CARs of the instant disclosure comprises an interleukin-2 receptor beta-chain (IL-2Rbeta or IL-2R-beta) cytoplasmic domain. In some embodiments, the IL-2Rbeta domain is truncated. In some embodiments, the IL-2Rbeta cytoplasmic domain comprises one or more STAT5-recruitment motifs. In some embodiments, the CAR comprises one or more STAT5-recruitment motifs outside the IL-2Rbeta cytoplasmic domain.

In some embodiments, the IL-2-Rbeta intracellular domain comprises an amino acid sequence having at least 80% identity, at least 90% identity, at least 95% identity, at least 99% identity or is identical to a sequence of

(SEQ ID NO: 245)
NCRNTGPWLKKVLKCNTPDPSKFFSQLSSEHGGDVQKWLSSPFPSSSFS
PGGLAPEISPLEVLERDKVTQLLPLNTDAYLSLQELQGQDPTHLV.

In some embodiments, the IL2R-beta intracellular domain comprises or consists essentially of SEQ ID NO: 245. In some embodiments, the IL-2R-beta intracellular domain is encoded by a nucleotide sequence having at least 80% identity, at least 90% identity, at least 95% identity, at least 99% identity or is identical to a sequence of

(SEQ ID NO: 246)
AACTGCAGGAACACCGGGCCATGGCTGAAGAAGGTCCTGAAGTGTAACA
CCCCAGACCCCTCGAAGTTCTTTTCCCAGCTGAGCTCAGAGCATGGAGG
CGACGTCCAGAAGTGGCTCTCTTCGCCCTTCCCCTCATCGTCCTTCAGC
CCTGGCGGCCTGGCACCTGAGATCTCGCCACTAGAAGTGCTGGAGAGGG
ACAAGGTGACGCAGCTGCTCCCCCTGAACACTGATGCCTACTTGTCTCT
CCAAGAACTCCAGGGTCAGGACCCAACTCACTTGGTG.

In some embodiments, the IL-2R-beta intracellular domain is encoded by SEQ ID NO: 246.

In an embodiment, the IL-2R-beta cytoplasmic domain comprises one or more STAT5-recruitment motifs. Exemplary STAT5-recruitment motifs are provided by Passerini et al. (2008) STAT5-signaling cytokines regulate the expression of FOXP3 in CD4+CD25+ regulatory T cells and CD4+CD25+ effector T cells. International Immunology, Vol. 20, No. 3, pp. 421-431, and by Kagoya et al. (2018) A novel chimeric antigen receptor containing a JAK-STAT signaling domain mediates superior antitumor effects. Nature Medicine doi:10.1038/nm.4478.

In some embodiments, the STAT5-recruitment motif(s) consists of the sequence Tyr-Leu-Ser-Leu (SEQ ID NO: 247).

In some embodiments, the CAR comprises an intracellular domain isolated or derived from CD28, 4-1BB and/or CD3z, or a combination thereof. In some embodiments, the intracellular domain of the CAR comprises a CD28 co-stimulatory domain, a 4-1BB costimulatory domain, and a CD3ζ activation domain. In some embodiments, the intracellular domain of the CAR comprises a sequence of RSKRSRLLHSDYMNMTPRRPGPTRKHYQPYAPPRDFAAYRSKRGRKKLLYIFKQP FMRPVQTTQEEDGCSCRFPEEEEGGCELRVKFSRSADAPAYKQGQNQLYNELNL GRREEYDVLDKRRGRDPEMGGKPRRKNPQEGLYNELQKDKMAEAYSEIGMKGE RRRGKGHDGLYQGLSTATKDTYDALHMQALPPR (SEQ ID NO: 489), or a sequence having at least 80% identity, at least 90% identity, at least 95% identity, at least 99% identity thereto.

In some embodiments, the intracellular domain of the CAR is encoded by: AGGAGCAAGCGGAGTCGACTGCTGCACAGCGACTACATGAACATGACCCCCC GGAGGCCTGGCCCCACCCGGAAGCACTACCAGCCCTACGCCCCTCCCAGGGAT TTCGCCGCCTACCGGAGCAAACGGGGCAGAAAGAAACTCCTGTATATATTCAA ACAACCATTTATGAGGCCAGTACAAACTACTCAAGAGGAAGATGGCTGTAGC TGCCGATTTCCAGAAGAAGAAGAAGGAGGATGTGAACTGAGAGTGAAGTTCA GCAGGAGCGCAGACGCCCCCGCGTACAAGCAGGGCCAGAACCAGCTCTATAA CGAGCTCAATCTAGGACGAAGAGAGGAGTACGATGTTTTGGACAAGCGTAGA GGCCGGGACCCTGAGATGGGGGGAAAGCCGAGAAGGAAGAACCCTCAGGAA GGCCTGTACAATGAACTGCAGAAAGATAAGATGGCGGAGGCCTACAGTGAGA TTGGGATGAAAGGCGAGCGCCGGAGGGGCAAGGGGCACGATGGCCTTTACCA GGGACTCAGTACAGCCACCAAGGACACCTACGACGCCCTTCACATGCAGGCC CTGCCCCCTCGC (SEQ ID NO: 490), or a sequence having at least 80% identity, at least 90% identity, at least 95% identity, at least 99% identity thereto. In some embodiments, the intracellular domain of the CAR is encoded by SEQ ID NO: 490.

In some embodiments, the intracellular domain of the CAR is encoded by: (SEQ ID NO: 21981), or a sequence having at least 80% identity, at least 90% identity, at least 95% identity, at least 99% identity thereto. In some embodiments, the intracellular domain of the CAR is encoded by SEQ ID NO: 21981.

Further Exemplary domains derived from CD28, 4-1BB and/or CD3z are provided as SEQ ID NOs: 248-253. In some embodiments, the CAR comprises an intracellular domain comprising a sequence of SEQ ID NOS: 248, 250 or 252, or a sequence having at least 80%, at least 85%, at least 90%, at least 95%, at least 97%, at least 99%, or that is identical thereto. In some embodiments, the CAR comprises an intracellular domain comprising a sequence of SEQ ID NOS: 248, 250 or 252.

The cytoplasmic domains within the cytoplasmic signaling portion of the CARs of the instant disclosure may be linked to each other in a random or specified order. Optionally, a short oligo- or polypeptide linker, for example between 2 and 10 amino acids in length may form the linkage. A glycine-serine doublet provides an example of a suitable linker. An exemplary linker comprises a sequence of GGGGSGGGGSGGGGSGG (SEQ ID NO: 491).

The cytoplasmic domains within the cytoplasmic signaling portion of the CARs of the instant disclosure may be linked to each other in a random or specified order. Optionally, a short oligo- or polypeptide linker, for example between 2 and 10 amino acids in length may form the linkage. A glycine-serine doublet provides an example of a suitable linker.

### T Cell Receptors (TCRs)

The disclosure provides a first, activator receptor and immune cells comprising same. In some embodiments, the first receptor is a T cell receptor (TCR).

Exemplary TCRs comprising intracellular domains for use in the instant disclosure are described in PCT/US2020/045250 filed on Sep. 6, 2020, the contents of which are incorporated herein by reference.

As used herein, a “TCR”, sometimes also called a “TCR complex” or “TCR/CD3 complex” refers to a protein complex comprising a TCR alpha chain, a TCR beta chain, and one or more of the invariant CD3 chains (zeta, gamma, delta and epsilon), sometimes referred to as subunits. The TCR alpha and beta chains can be disulfide-linked to function as a heterodimer to bind to peptide-MHC complexes. Once the TCR alpha/beta heterodimer engages peptide-MHC, conformational changes in the TCR complex in the associated invariant CD3 subunits are induced, which leads to their phosphorylation and association with downstream proteins, thereby transducing a primary stimulatory signal. In an exemplary TCR complex, the TCR alpha and TCR beta polypeptides form a heterodimer, CD3 epsilon and CD3 delta form a heterodimer, CD3 epsilon and CD3 gamma for a heterodimer, and two CD3 zeta form a homodimer.

Any suitable ligand binding domain may be fused to an extracellular domain, hinge domain or transmembrane of the TCRs described herein. For example, the ligand binding domain can be an antigen binding domain of an antibody or TCR, or comprise an antibody fragment, a Vβ only domain, a linear antibody, a single-chain variable fragment (scFv), or a single domain antibody (sdAb).

### Components of Inhibitory Receptors

The disclosure provides a second receptor that is an inhibitory receptor. The inhibitory receptor may comprise an extracellular antigen binding domain that binds to and recognizes the non-target antigen or a peptide derivative thereof in a MHC-I complex. In some embodiments, the second inhibitory receptor is a chimeric antigen receptor or a TCR. Any suitable inhibitory receptor architecture is envisaged as within the scope of the instant disclosure.

In some embodiments, for example in the second inhibitory receptors of the disclosure which provide an inhibitory signal, the inhibitory signal is transmitted through the intracellular domain of the receptor. In some embodiments, the inhibitory receptor comprises an inhibitory intracellular domain. In some embodiments, the inhibitory receptor is a CAR comprising an inhibitory intracellular domain. In some embodiments, the inhibitory receptor is a TCR comprising an inhibitory intracellular domain. Exemplary inhibitory TCR are described in WO2021096868, filed on Nov. 10, 2020, PCT/US2020/045228 filed on Sep. 6, 2020, PCT/US2020/064607, filed on Dec. 11, 2020, PCT/US2021/029907, filed on Apr. 29, 2021, the contents of each of which are incorporated herein by reference.

The term “inhibitory receptor,” as used herein refers to a ligand binding domain that is fused to an intracellular signaling domain capable of transducing an inhibitory signal that inhibits or suppresses the immune activity of an immune cell. Inhibitory receptors have immune cell inhibitory potential, and are distinct and distinguishable from CARs, which are receptors with immune cell activating potential. For example, CARs are activating receptors as they include intracellular stimulatory and/or co-stimulatory domains. Inhibitory receptors are inhibiting receptors that contain intracellular inhibitory domains.

As used herein “inhibitory signal” refers to signal transduction or changes in protein expression in an immune cell resulting in suppression of an immune response (e.g., decrease in cytokine production or reduction of immune cell activation). Inhibition or suppression of an immune cell can selective and/or reversible, or not selective and/or reversible. Inhibitory receptors are responsive to non-target antigens (e.g. HLA-A*02). For example, when a non-target antigen (e.g. HLA-A*02) binds to or contacts the inhibitory receptor, the inhibitory receptor is responsive and activates an inhibitory signal in the immune cell expressing the inhibitory receptor upon binding of the non-target antigen by the extracellular ligand binding domain of the inhibitory receptor.

Inhibitory receptors of the disclosure may comprise an extracellular ligand binding domain. Any type of ligand binding domain that can regulate the activity of a receptor in a ligand dependent manner is envisaged as within the scope of the instant disclosure.

In some embodiments, the ligand binding domain is an antigen binding domain. Exemplary antigen binding domains include, inter alia, scFv, SdAb, Vβ-only domains, and TCR antigen binding domains derived from the TCR α and β chain variable domains.

Any type of antigen binding domain is envisaged as within the scope of the instant disclosure.

In some embodiments, the extracellular ligand binding domain of the second receptor is an scFv.

In some embodiments, the extracellular ligand binding domain of the second receptor is fused to the extracellular domain of an inhibitory CAR.

In some embodiments, the inhibitory receptors of the present disclosure comprise an extracellular hinge region. Exemplary hinges can be isolated or derived from IgD and CD8 domains, for example IgG1. In some embodiments, the hinge is isolated or derived from CD8a or CD28.

The inhibitory receptors of the present disclosure can be designed to comprise a transmembrane domain that is fused to the extracellular domain of the inhibitory receptor. In some instances, the transmembrane domain can be selected or modified by amino acid substitution to avoid binding of such domains to the transmembrane domains of the same or different surface membrane proteins to minimize interactions with other members of the receptor complex.

The transmembrane domain may be derived either from a natural or from a synthetic source. Where the source is natural, the domain may be derived from any membrane-bound or transmembrane protein. Transmembrane regions may be isolated or derived from (i.e. comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154, or from an immunoglobulin such as IgG4. Alternatively the transmembrane domain may be synthetic, in which case it will comprise predominantly hydrophobic residues such as leucine and valine. In some embodiments, a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the intracellular domain of the inhibitory receptor. A glycine-serine doublet provides a particularly suitable linker.

The disclosure provides an inhibitory receptor comprising an intracellular domain. The intracellular domain of the inhibitory receptors of the instant disclosure is responsible for inhibiting activation of the immune cells comprising the inhibitory receptor, which would otherwise be activated in response to activation signals by the first receptor. In some embodiments, the inhibitory intracellular domain comprises an immunoreceptor tyrosine-based inhibitory motif (ITIM). In some embodiments, the inhibitory intracellular domain comprising an ITIM can be isolated or derived from an immune checkpoint inhibitor such as CTLA-4 and PD-1. CTLA-4 and PD-1 are immune inhibitory receptors expressed on the surface of T cells, and play a pivotal role in attenuating or terminating T cell responses.

In some embodiments, an inhibitory intracellular domain is isolated from human tumor necrosis factor related apoptosis inducing ligand (TRAIL) receptor and CD200 receptor 1. In some embodiments, the TRAIL receptor comprises TR10A, TR10B or TR10D.

In some embodiments, an inhibitory intracellular domain is isolated from phosphoprotein membrane anchor with glycosphingolipid microdomains 1 (PAG1). In some embodiments, an inhibitory intracellular domain is isolated from leukocyte immunoglobulin like receptor B1 (LILRB1).

In some embodiments, the inhibitory domain is isolated or derived from a human protein, for example a human TRAIL receptor, CTLA-4, PD-1, PAG1 or LILRB1 protein.

In some embodiments, the inhibitory domain comprises an intracellular domain, a transmembrane or a combination thereof. In some embodiments, the inhibitory domain comprises an intracellular domain, a transmembrane domain, a hinge region or a combination thereof.

In some embodiments, the inhibitory domain is isolated or derived from killer cell immunoglobulin like receptor, three Ig domains and long cytoplasmic tail 2 (KIR3DL2), killer cell immunoglobulin like receptor, three Ig domains and long cytoplasmic tail 3 (KIR3DL3), leukocyte immunoglobulin like receptor B1 (LIR1, also called LIR-1 and LILRB1), programmed cell death 1 (PD-1), Fc gamma receptor JIB (FcgRIIB), killer cell lectin like receptor K1 (NKG2D), CTLA-4, a domain containing a synthetic consensus ITIM, a ZAP70 SH2 domain (e.g., one or both of the N and C terminal SH2 domains), or ZAP70 KI_K369A (kinase inactive ZAP70).

In some embodiments, the inhibitory domain is isolated or derived from a human protein.

In some embodiments, the second, inhibitory receptor comprises an inhibitory domain. In some embodiments, the second, inhibitory receptor comprises an inhibitory intracellular domain and/or an inhibitory transmembrane domain. In some embodiments, the inhibitory intracellular domain is fused to an intracellular domain of an inhibitory receptor. In some embodiments, the inhibitory intracellular domain is fused to the transmembrane domain of an inhibitory receptor.

In some embodiments, the second, inhibitory receptor comprises a cytoplasmic domain, a transmembrane domain, and an extracellular domain or a portion thereof isolated or derived isolated or derived from the same protein, for example an ITIM containing protein. In some embodiments, the second, inhibitory receptor comprises a hinge region isolated or derived from isolated or derived from the same protein as the intracellular domain and/or transmembrane domain, for example an ITIM containing protein.

In some embodiments, the second receptor is a TCR comprising an inhibitory domain (an inhibitory TCR). In some embodiments, the inhibitory TCR comprises an inhibitory intracellular domain and/or an inhibitory transmembrane domain. In some embodiments, the inhibitory intracellular domain is fused to the intracellular domain of TCR alpha, TCR beta, CD3 delta, CD3 gamma or CD3 epsilon or a portion thereof a TCR. In some embodiments, the inhibitory intracellular domain is fused to the transmembrane domain of TCR alpha, TCR beta, CD3 delta, CD3 gamma or CD3 epsilon.

In some embodiments, the second receptor is a TCR comprising an inhibitory domain (an inhibitory TCR).

Components of LILRB1-based Receptors

The present disclosure describes inhibitory receptors having one or more domains from Leukocyte immunoglobulin-like receptor subfamily B member 1 (LILRB1, or LIR1). In some embodiments, the inhibitory receptor comprises a LILRB1 intracellular domain or a functional variant thereof.

Leukocyte immunoglobulin-like receptor subfamily B member 1 (LILRB1), also known as Leukocyte immunoglobulin-like receptor B1, as well as ILT2, LIR1, MIR7, PIRB, CD85J, ILT-2 LIR-1, MIR-7 and PIR-B, is a member of the leukocyte immunoglobulin-like receptor (LIR) family. The LILRB1 protein belongs to the subfamily B class of LIR receptors. These receptors contain two to four extracellular immunoglobulin domains, a transmembrane domain, and two to four cytoplasmic immunoreceptor tyrosine-based inhibitory motifs (ITIMs). The LILRB1 receptor is expressed on immune cells, where it binds to MHC class I molecules on antigen-presenting cells and transduces a negative signal that inhibits stimulation of an immune response. LILRB1 is thought to regulate inflammatory responses, as well as cytotoxicity, and to play a role in limiting auto-reactivity. Multiple transcript variants encoding different isoforms of LILRB1 exist, all of which are contemplated as within the scope of the instant disclosure.

In various embodiments, a chimeric antigen receptor is provided, comprising a polypeptide, wherein the polypeptide comprises one or more of: an LILRB1 hinge domain or functional fragment or variant thereof, an LILRB1 transmembrane domain or a functional variant thereof, and an LILRB1 intracellular domain or an intracellular domain comprising at least one, or at least two immunoreceptor tyrosine-based inhibitory motifs (ITIMs), wherein each ITIM is independently selected from NLYAAV (SEQ ID NO: 256), VTYAEV (SEQ ID NO: 257), VTYAQL (SEQ ID NO: 258), and SIYATL (SEQ ID NO: 259).

### Intracellular Domain

The disclosure provides inhibitory receptors, the inhibitory receptors comprising a polypeptide. In some embodiments, the polypeptide comprises an intracellular domain. In some embodiments, the inhibitory receptor comprises a LILRB1 intracellular domain or a functional variant thereof.

As used herein an “immunoreceptor tyrosine-based inhibitory motif” or “ITIM” refers to a conserved sequence of amino acids with a consensus sequence of S/I/V/LxYxxI/V/L (SEQ ID NO: 260), or the like, that is found in the cytoplasmic tails of many inhibitory receptors of the immune system. After ITIM-possessing inhibitory receptors interact with their antigen, the ITIM motif is phosphorylated, allowing the inhibitory receptor to recruit other enzymes, such as the phosphotyrosine phosphatases SHP-1 and SHP-2, or the inositol-phosphatase called SHIP.

In some embodiments, the polypeptide comprises an intracellular domain comprising at least one immunoreceptor tyrosine-based inhibitory motif (ITIM), at least two ITIMs, at least 3 ITIMs, at least 4 ITIMs, at least 5 ITIMs or at least 6 ITIMs. In some embodiments, the intracellular domain has 1, 2, 3, 4, 5, or 6 ITIMs.

In some embodiments, the polypeptide comprises an intracellular domain comprising at least one ITIM, or at least two ITIMs selected from the group of ITIMs consisting of NLYAAV (SEQ ID NO: 256), VTYAEV (SEQ ID NO: 257), VTYAQL (SEQ ID NO: 258), and SIYATL (SEQ ID NO: 259).

In some embodiments, the intracellular domain comprises both ITIMs NLYAAV (SEQ ID NO: 256) and VTYAEV (SEQ ID NO: 257). In some embodiments, the intracellular domain comprises a sequence at least 95% identical to SEQ ID NO: 260. In some embodiments, the intracellular domain comprises or consists essentially of a sequence identical to SEQ ID NO: 281.

In some embodiments, the intracellular domain comprises both ITIMs VTYAEV (SEQ ID NO: 257) and VTYAQL (SEQ ID NO: 258). In some embodiments, the intracellular domain comprises a sequence at least 95% identical to SEQ ID NO: 261. In some embodiments, the intracellular domain comprises or consists essentially of a sequence identical to SEQ ID NO: 261.

In some embodiments, the intracellular domain comprises both ITIMs VTYAQL (SEQ ID NO: 258) and SIYATL (SEQ ID NO: 259). In some embodiments, the intracellular domain comprises a sequence at least 95% identical to SEQ ID NO: 262. In some embodiments, the intracellular domain comprises or consists essentially of a sequence identical to SEQ ID NO: 262.

In some embodiments, the intracellular domain comprises the ITIMs NLYAAV (SEQ ID NO: 256), VTYAEV (SEQ ID NO: 257), and VTYAQL (SEQ ID NO: 258). In some embodiments, the intracellular domain comprises a sequence at least 95% identical to SEQ ID NO: 263. In some embodiments, the intracellular domain comprises or consists essentially of a sequence identical to SEQ ID NO: 263.

In some embodiments, the intracellular domain comprises the ITIMs VTYAEV (SEQ ID NO: 257), VTYAQL (SEQ ID NO: 258), and SIYATL (SEQ ID NO: 259). In some embodiments, the intracellular domain comprises a sequence at least 95% identical to SEQ ID NO: 264. In some embodiments, the intracellular domain comprises or consists essentially of a sequence identical to SEQ ID NO: 264.

In some embodiments, the intracellular domain comprises the ITIMs NLYAAV (SEQ ID NO: 256), VTYAEV (SEQ ID NO: 257), VTYAQL (SEQ ID NO: 258), and SIYATL (SEQ ID NO: 259). In embodiments, the intracellular domain comprises a sequence at least 95% identical to SEQ ID NO: 265. In some embodiments, the intracellular domain comprises or consists essentially of a sequence identical to SEQ ID NO: 265.

In some embodiments, the intracellular domain comprises a sequence at least 95% identical to the LILRB1 intracellular domain (SEQ ID NO: 266). In some embodiments, the intracellular domain comprises or consists essentially of a sequence identical to the LILRB1 intracellular domain (SEQ ID NO: 266).

LILRB1 intracellular domains or functional variants thereof of the disclosure can have at least 1, at least 2, at least 4, at least 4, at least 5, at least 6, at least 7, or at least 8 ITIMs. In some embodiments, the LILRB1 intracellular domain or functional variant thereof has 2, 3, 4, 5, or 6 ITIMs.

In particular embodiments, the polypeptide comprises an intracellular domain comprising at least two, three, four, five, or six immunoreceptor tyrosine-based inhibitory motifs (ITIMs), wherein each ITIM is independently selected from NLYAAV (SEQ ID NO: 256), VTYAEV (SEQ ID NO: 257), VTYAQL (SEQ ID NO: 258), and SIYATL (SEQ ID NO: 259).

In particular embodiments, the polypeptide comprises an intracellular domain comprising three immunoreceptor tyrosine-based inhibitory motifs (ITIMs), wherein each ITIM is independently selected from NLYAAV (SEQ ID NO: 256), VTYAEV (SEQ ID NO: 257), VTYAQL (SEQ ID NO: 258), and SIYATL (SEQ ID NO: 259).

In particular embodiments, the polypeptide comprises an intracellular domain comprising four immunoreceptor tyrosine-based inhibitory motifs (ITIMs), wherein each ITIM is independently selected from NLYAAV (SEQ ID NO: 256), VTYAEV (SEQ ID NO: 257), VTYAQL (SEQ ID NO: 258), and SIYATL (SEQ ID NO: 259).

In particular embodiments, the polypeptide comprises an intracellular domain comprising five immunoreceptor tyrosine-based inhibitory motifs (ITIMs), wherein each ITIM is independently selected from NLYAAV (SEQ ID NO: 256), VTYAEV (SEQ ID NO: 257), VTYAQL (SEQ ID NO: 258), and SIYATL (SEQ ID NO: 259).

In particular embodiments, the polypeptide comprises an intracellular domain comprising six immunoreceptor tyrosine-based inhibitory motifs (ITIMs), wherein each ITIM is independently selected from NLYAAV (SEQ ID NO: 256), VTYAEV (SEQ ID NO: 257), VTYAQL (SEQ ID NO: 258), and SIYATL (SEQ ID NO: 259).

In particular embodiments, the polypeptide comprises an intracellular domain comprising at least seven immunoreceptor tyrosine-based inhibitory motifs (ITIMs), wherein each ITIM is independently selected from NLYAAV (SEQ ID NO: 256), VTYAEV (SEQ ID NO: 257), VTYAQL (SEQ ID NO: 258), and SIYATL (SEQ ID NO: 259).

### Transmembrane Domain

The disclosure provides inhibitory receptors the receptors comprising a polypeptide. In some embodiments, the polypeptide comprises a transmembrane domain. In some embodiments, the inhibitory receptor comprises a LILRB1 hinge domain, or a functional variant thereof. In some embodiments, the inhibitory receptor comprises a LILRB1 transmembrane domain, or a functional variant thereof. In some embodiments, the inhibitory receptor comprises LILRB1 hinge and transmembrane domains, or functional variants thereof.

A “transmembrane domain”, as used herein, refers to a domain of a protein that spans membrane of the cell. Transmembrane domains typically consist predominantly of non-polar amino acids, and may traverse the lipid bilayer once or several times. Transmembrane domains usually comprise alpha helices, a configuration which maximizes internal hydrogen bonding.

Transmembrane domains isolated or derived from any source are envisaged as within the scope of the fusion proteins of the disclosure.

In particular embodiments, the polypeptide comprises an LILRB1 transmembrane domain or a functional variant thereof.

In some embodiments, the LILRB1 transmembrane domain or a functional variant thereof comprises a sequence at least 95% identical, at least 96% identical, at least 97% identical, at least 98% identical or at least 99% to SEQ ID NO: 269. In some embodiments, the LILRB1 transmembrane domain or a functional variant thereof comprises a sequence at least 95% identical to SEQ ID NO: 269. In some embodiments, the LILRB1 transmembrane domain comprises a sequence identical to SEQ ID NO: 269. In embodiments, the LILRB1 transmembrane domain consists essentially of a sequence identical to SEQ ID NO: 269.

In some embodiments of the chimeric antigen receptors of the disclosure, the transmembrane domain is not a LILRB1 transmembrane domain. In some embodiments, the transmembrane domain is one that is associated with one of the other domains of the fusion protein, or isolated or derived from the same protein as one of the other domains of the fusion protein.

The transmembrane domain may be derived either from a natural or from a recombinant source. Where the source is natural, the domain may be derived from any membrane-bound or transmembrane protein. Exemplary transmembrane domains may include at least the transmembrane region(s) of e.g., the alpha, beta or zeta chain of the TCR, CD3 delta, CD3 epsilon or CD3 gamma, CD28, CD3 epsilon, CD45, CD4, CD5, CD8, CD9, CD16, CD22, CD33, CD3γ, CD64, CD80, CD86, CD134, CD137, CD154.

In some embodiments, the transmembrane domain can be attached to the extracellular region chimeric antigen receptor, e.g., the antigen-binding domain or antigen binding domain, via a hinge, e.g., a hinge from a human protein. For example, in some embodiments, the hinge can be a human immunoglobulin (Ig) hinge, e.g., an IgG4 hinge, a CD8a hinge or an LILRB1 hinge.

### Hinge Domain

The disclosure provides inhibitory receptors, the receptors comprising a polypeptide. In some embodiments, the polypeptide comprises a hinge domain. In some embodiments, the hinge domain is a LILRB1 hinge domain or a functional variant thereof.

The LILRB1 protein has four immunoglobulin (Ig) like domains termed D1, D2, D3 and D4. In some embodiments, the LILRB1 hinge domain comprises an LILRB1 D3D4 domain or a functional variant thereof. In some embodiments, the LILRB1 D3D4 domain comprises a sequence at least 95%, at least 96%, at least 97%, at least 98%, at least 99% or identical to SEQ ID NO: 274. In some embodiments, the LILRB1 D3D4 domain comprises or consists essentially of SEQ ID NO: 274.

In some embodiments, the polypeptide comprises the LILRB1 hinge domain or functional fragment or variant thereof. In embodiments, the LILRB1 hinge domain or functional fragment or variant thereof comprises a sequence at least 95%, at least 96%, at least 97%, at least 98%, at least 99% identical or identical to SEQ ID NO: 275, SEQ ID NO: 274, or SEQ ID NO: 276. In embodiments, the LILRB1 hinge domain or functional fragment or variant thereof comprises a sequence at least 95% identical to SEQ ID NO: 275, SEQ ID NO: 274, or SEQ ID NO: 276.

In some embodiments, the LILRB1 hinge domain comprises a sequence identical to SEQ ID NO: 275, SEQ ID NO: 274, or SEQ ID NO: 276.

In some embodiments, the LILRB1 hinge domain consists essentially of a sequence identical to SEQ ID NO: 275, SEQ ID NO: 274, or SEQ ID NO: 276.

In some embodiments the chimeric antigen receptors of the disclosure, the polypeptide comprises a hinge that is not isolated or derived from LILRB1.

In some embodiments, the hinge is isolated or derived from CD8a or CD28. In some embodiments, the CD8a hinge comprises an amino acid sequence having at least 80% identity, at least 90% identity, at least 95% identity, at least 99% identity or is identical to a sequence of SEQ ID NO: 219. In some embodiments, the CD8a hinge comprises SEQ ID NO: 219. In some embodiments, the CD8a hinge consists essentially of SEQ ID NO: 219. In some embodiments, the CD8a hinge is encoded by a nucleotide sequence having at least 80% identity, at least 90% identity, at least 95% identity, at least 99% identity or is identical to a sequence of SEQ ID NO: 220.

In some embodiments, the CD28 hinge comprises an amino acid sequence having at least 80% identity, at least 90% identity, at least 95% identity, at least 99% identity or is identical to a sequence of SEQ ID NO: 221. In some embodiments, the CD28 hinge comprises or consists essentially of SEQ ID NO: 221. In some embodiments, the CD28 hinge is encoded by a nucleotide sequence having at least 80% identity, at least 90% identity, at least 95% identity, at least 99% identity or is identical to a sequence of SEQ ID NO: 222.

### Combinations of LILRB1 Domains

In some embodiments, the inhibitory receptors of the disclosure comprise a polypeptide comprising more than one LILRB1 domain or functional equivalent thereof. For example, in some embodiments, the polypeptide comprises an LILRB1 transmembrane domain and intracellular domain, or an LILRB1 hinge domain, transmembrane domain and intracellular domain.

In particular embodiments, the polypeptide comprises an LILRB1 hinge domain or functional fragment or variant thereof, and the LILRB1 transmembrane domain or a functional variant thereof. In some embodiments, the polypeptide comprises a sequence at least 95% identical, at least 96% identical, at least 97% identical, at least 98% identical, at least 99% identical or identical to SEQ ID NO: 277. In some embodiments, the polypeptide comprises a sequence at least 95% identical to SEQ ID NO: 277. In some embodiments, the polypeptide comprises a sequence identical to SEQ ID NO: 277.

In further embodiments, the polypeptide comprises: the LILRB1 transmembrane domain or a functional variant thereof, and an LILRB1 intracellular domain and/or an intracellular domain comprising at least one immunoreceptor tyrosine-based inhibitory motif (ITIM), wherein the ITIM is selected from NLYAAV (SEQ ID NO: 256), VTYAEV (SEQ ID NO: 257), VTYAQL (SEQ ID NO: 258), and SIYATL (SEQ ID NO: 259). In some embodiments, the polypeptide comprises the LILRB1 transmembrane domain or a functional variant thereof, and an LILRB1 intracellular domain and/or an intracellular domain comprising at least two ITIM, wherein each ITIM is independently selected from NLYAAV (SEQ ID NO: 256), VTYAEV (SEQ ID NO: 257), VTYAQL (SEQ ID NO: 258), and SIYATL (SEQ ID NO: 259).

In some embodiments, the polypeptide comprises a LILRB1 transmembrane domain and intracellular domain. In some embodiments, the polypeptide comprises a sequence at least 95% identical, at least 96% identical, at least 97% identical, at least 98% identical, at least 99% identical or identical to SEQ ID NO: 278. In some embodiments, the polypeptide comprises a sequence at least 95% identical to SEQ ID NO: 278. In some embodiments, the polypeptide comprises a sequence identical to SEQ ID NO: 278.

In preferred embodiments, the polypeptide comprises: an LILRB1 hinge domain or functional fragment or variant thereof; an LILRB1 transmembrane domain or a functional variant thereof; and an LILRB1 intracellular domain and/or an intracellular domain comprising at least two immunoreceptor tyrosine-based inhibitory motifs (ITIMs), wherein each ITIM is independently selected from NLYAAV (SEQ ID NO: 256), VTYAEV (SEQ ID NO: 257), VTYAQL (SEQ ID NO: 258), and SIYATL (SEQ ID NO: 259).

In some embodiments, the polypeptide comprises a sequence at least 95% identical to SEQ ID NO: 254 or SEQ ID NO: 279, or at least 99% identical to SEQ ID NO: 254 or SEQ ID NO: 279, or identical to SEQ ID NO: 254 or SEQ ID NO: 279.

In some embodiments, the polypeptide comprises a sequence at least 99% identical to SEQ ID NO: 277, or at least 99% identical to SEQ ID NO: 277, or identical to SEQ ID NO: 277.

In some embodiments, the polypeptide comprises a sequence at least 99% identical to SEQ ID NO: 278, or at least 99% identical to SEQ ID NO: 278, or identical to SEQ ID NO: 278.

In some embodiments, the inhibitory receptor comprises a LIRLRB1 hinge, transmembrane and intracellular domain. In some embodiments, the LIRLRB1 hinge, transmembrane and intracellular domain comprises a sequence of SEQ ID NO: 254, or a sequence at 90%, at least 95% identical, at least 96% identical, at least 97% identical, at least 98% identical, at least 99% identical or identical thereto.

TABLE 6B
Exemplary Inhibitory Receptor Hinge, Transmembrane and Intracellular
Domains

| Protein Sequence | DNA Sequence |
|---|---|
| YGSQSSKPYLLTHPS | TACGGCTCACAGAGCTCCAAACCCTACCTGCTGACTCACCCCA |
| DPLELVVSGPSGGPS | GTGACCCCCTGGAGCTCGTGGTCTCAGGACCGTCTGGGGGCC |
| SPTTGPTSTSGPEDQ | CCAGCTCCCCGACAACAGGCCCCACCTCCACATCTGGCCCTGA |
| PLTPTGSDPQSGLGR | GGACCAGCCCCTCACCCCCACCGGGTCGGATCCCCAGAGTGG |
| HLGVVIGILVAVILL | TCTGGGAAGGCACCTGGGGGTTGTGATCGGCATCTTGGTGGC |
| LLLLLLLFLILRHRR | CGTCATCCTACTGCTCCTCCTCCTCCTCCTCCTCTTCCTCATCC |
| QGKHWTSTQRKAD | TCCGACATCGACGTCAGGGCAAACACTGGACATCGACCCAGA |
| FQHPAGAVGPEPTD | GAAAGGCTGATTTCCAACATCCTGCAGGGGCTGTGGGGCCAG |
| RGLQWRSSPAADAQ | AGCCCACAGACAGAGGCCTGCAGTGGAGGTCCAGCCCAGCTG |
| EENLYAAVKHTQPE | CCGATGCCCAGGAAGAAAACCTCTATGCTGCCGTGAAGCACA |
| DGVEMDTRSPHDED | CACAGCCTGAGGATGGGGTGGAGATGGACACTCGGAGCCCAC |
| PQAVTYAEVKHSRP | ACGATGAAGACCCCCAGGCAGTGACGTATGCCGAGGTGAAAC |
| RREMASPPSPLSGEF | ACTCCAGACCTAGGAGAGAAATGGCCTCTCCTCCTTCCCCACT |
| LDTKDRQAEEDRQ | GTCTGGGGAATTCCTGGACACAAAGGACAGACAGGCGGAAG |
| MDTEAAASEAPQDV | AGGACAGGCAGATGGACACTGAGGCTGCTGCATCTGAAGCCC |
| TYAQLHSLTLRREA | CCCAGGATGTGACCTACGCCCAGCTGCACAGCTTGACCCTCA |
| TEPPPSQEGPSPAVP | GACGGGAGGCAACTGAGCCTCCTCCATCCCAGGAAGGGCCCT |
| SIYATLAIH (SEQ ID | CTCCAGCTGTGCCCAGCATCTACGCCACTCTGGCCATCCACTA |
| NO: 254) | G (SEQ ID NO: 255) |

Additional sequences for the LILRB31 based inhibitory receptors of the disclosure are shown in Table 7 below.

TABLE 7
Polypeptide Sequences for Elements of Illustrative Inhibitory Receptors

| Name | Sequence |
|---|---|
| LILRB1 | MTPILTVLICLGLSLGPRTHVQAGHLPKPTLWAEPGSVITQGSP |
| | VTLRCQGGQETQEYRLYREKKTALWITRIPQELVKKGQFPIPSI |
| | TWEHAGRYRCYYGSDTAGRSESSDPLELVVTGAYIKPTLSAQP |
| | SPVVNSGGNVILQCDSQVAFDGFSLCKEGEDEHPQCLNSQPHA |
| | RGSSRAIFSVGPVSPSRRWWYRCYAYDSNSPYEWSLPSDLLEL |
| | LVLGVSKKPSLSVQPGPIVAPEETLTLQCGSDAGYNRFVLYKD |
| | GERDFLQLAGAQPQAGLSQANFTLGPVSRSYGGQYRCYGAHN |
| | LSSEWSAPSDPLDILIAGQFYDRVSLSVQPGPTVASGENVTLLC |
| | QSQGWMQTFLLTKEGAADDPWRLRSTYQSQKYQAEFPMGPV |
| | TSAHAGTYRCYGSQSSKPYLLTHPSDPLELVVSGPSGGPSSPT |
| | TGPTSTSGPEDQPLTPTGSDPQSGLGRHLGVVIGILVAVILLLLL |
| | LLLLFLILRHRRQGKHWTSTQRKADFQHPAGAVGPEPTDRGL |
| | QWRSSPAADAQEENLYAAVKHTQPEDGVEMDTRSPHDEDPQ |
| | AVTYAEVKHSRPRREMASPPSPLSGEFLDTKDRQAEEDRQMD |
| | TEAAASEAPQDVTYAQLHSLTLRREATEPPPSQEGPSPAVPSIY |
| | ATLAIHPSQEGPSPAVPSIYATLAIH |
| | SEQ ID NO: 280 |
| LILRB1 hinge- | YGSQSSKPYLLTHPSDPLELVVSGPSGGPSSPTTGPTSTSGPED |
| transmembrane- | QPLTPTGSDPQSGLGRHLGVVIGILVAVILLLLLLLLLFLILRH |
| intracellular | RRQGKHWTSTQRKADFQHPAGAVGPEPTDRGLQWRSSPAAD |
| domain | AQEENLYAAVKHTQPEDGVEMDTRSPHDEDPQAVTYAEVKH |
| | SRPRREMASPPSPLSGEFLDTKDRQAEEDRQMDTEAAASEAPQ |
| | DVTYAQLHSLTLRREATEPPPSQEGPSPAVPSIYATLAIH |
| | SEQ ID NO: 254 |
| LILRB1 hinge- | VVSGPSGGPSSPTTGPTSTSGPEDQPLTPTGSDPQSGLGRHLGV |
| transmembrane- | VIGILVAVILLLLLLLLLFLILRHRRQGKHWTSTQRKADFQHP |
| intracellular | AGAVGPEPTDRGLQWRSSPAADAQEENLYAAVKHTQPEDGV |
| domain (w/o | EMDTRSPHDEDPQAVTYAEVKHSRPRREMASPPSPLSGEFLDT |
| YGSQSSKPYLL | KDRQAEEDRQMDTEAAASEAPQDVTYAQLHSLTLRREATEPP |
| THPSDPLEL) | PSQEGPSPAVPSIYATLAIH |
| | SEQ ID NO: 279 |
| LILRB1 hinge | YGSQSSKPYLLTHPSDPLELVVSGPSGGPSSPTTGPTSTSGPED |
| domain | QPLTPTGSDPQSGLGRHLG |
| | SEQ ID NO: 275 |
| LILRB1 | VVIGILVAVILLLLLLLLLFLIL |
| transmembrane | SEQ ID NO: 269 |
| domain | |
| LILRB1 | RHRRQGKHWTSTQRKADFQHPAGAVGPEPTDRGLQWRSSPA |
| intracellular | ADAQEENLYAAVKHTQPEDGVEMDTRSPHDEDPQAVTYAEV |
| domain | KHSRPRREMASPPSPLSGEFLDTKDRQAEEDRQMDTEAAASEA |
| | PQDVTYAQLHSLTLRREATEPPPSQEGPSPAVPSIYATLAIH |
| | SEQ ID NO: 266 |
| ITIM1 | NLYAAV |
| | SEQ ID NO: 256 |
| ITIM2 | VTYAEV |
| | SEQ ID NO: 257 |
| ITIM3 | VTYAQL |
| | SEQ ID NO: 258 |
| ITIM4 | SIYATL |
| | SEQ ID NO: 259 |
| ITIM1-2 | NLYAAVKHTQPEDGVEMDTRSPHDEDPQAVTYAEV |
| | SEQ ID NO: 281 |
| ITIM2-3 | VTYAEVKHSRPRREMASPPSPLSGEFLDTKDRQAEEDRQMDT |
| | EAAASEAPQDVTYAQL |
| | SEQ ID NO: 261 |
| ITIM3-4 | VTYAQLHSLTLRREATEPPPSQEGPSPAVPSIYATL |
| | SEQ ID NO: 262 |
| ITIM1-3 | NLYAAVKHTQPEDGVEMDTRSPHDEDPQAVTYAEVKHSRPR |
| | REMASPPSPLSGEFLDTKDRQAEEDRQMDTEAAASEAPQDVT |
| | YAQL |
| | SEQ ID NO: 263 |
| ITIM2-4 | VTYAEVKHSRPRREMASPPSPLSGEFLDTKDRQAEEDRQMDT |
| | EAAASEAPQDVTYAQLHSLTLRREATEPPPSQEGPSPAVPSIYA |
| | TL |
| | SEQ ID NO: 264 |
| ITIM1-4 | NLYAAVKHTQPEDGVEMDTRSPHDEDPQAVTYAEVKHSRPR |
| | REMASPPSPLSGEFLDTKDRQAEEDRQMDTEAAASEAPQDVT |
| | YAQLHSLTLRREATEPPPSQEGPSPAVPSIYATL |
| | SEQ ID NO: 265 |
| D3D4 domain | YGSQSSKPYLLTHPSDPLEL |
| | SEQ ID NO: 274 |
| Short hinge | VVSGPSGGPSSPTTGPTSTSGPEDQPLTPTGSDPQSGLGRHLG |
| | SEQ ID NO: 276 |
| Hinge (iTIM | YGSQSSKPYLLTHPSDPLELVVSGPSGGPSSPTTGPTSTSGPEDQ |
| hinge) | PLTPTGSDPQSGLGRHLGV (SEQ ID NO: 282) |
| Short hinge 2 | VVSGPSGGPSSPTTGPTSTSGPEDQPLTPTGSDPQSGLGRHLGV |
| | (SEQ ID NO: 283) |
| Long hinge 1 | AGSGGSGGSGGSPVPSTPPTPSPSTPPTPSPSGGSGNSSGSGGSP |
| | VPSTPPTPSPSTPPTPSPSASV (SEQ ID NO: 284) |
| Long hinge 2 | AGSGGSGGSGGSPVPSTPPTNSSSTPPTPSPSPVPSTPPTNSSSTP |
| | PTPSPSPVPSTPPTNSSSTPPTPSPSASV (SEQ ID NO: 285) |
| 2X short hinge | VVSGPSGGPSSPTTGPTSTSGPEDQPLTPTGSDPQSGLGRHVVS |
| | GPSGGPSSPTTGPTSTSGPEDQPLTPTGSDPQSGLGRHLGV |
| | (SEQ ID NO: 286) |
| Hinge (truncated) | TTGPTSTSGPEDQPLTPTGSDPQSGLGRHLGV (SEQ ID NO: 287) |
| Hinge- | YGSQSSKPYLLTHPSDPLELVVSGPSGGPSSPTTGPTSTSGPED |
| transmembrane | QPLTPTGSDPQSGLGRHLGVVIGILVAVILLLLLLLLLFLIL |
| | SEQ ID NO: 277 |
| Transmembrane- | VVIGILVAVILLLLLLLLLFLILRHRRQGKHWTSTQRKADFQ |
| intracellular | HPAGAVGPEPTDRGLQWRSSPAADAQEENLYAAVKHTQPED |
| domain | GVEMDTRSPHDEDPQAVTYAEVKHSRPRREMASPPSPLSGEFL |
| | DTKDRQAEEDRQMDTEAAASEAPQDVTYAQLHSLTLRREATE |
| | PPPSQEGPSPAVPSIYATLAIH |
| | SEQ ID NO: 278 |

### Assays

Provided herein are assays that can be used to measure the activity of the engineered receptors of the disclosure.

The activity of engineered receptors can be assayed using a cell line engineered to express a reporter of receptor activity such as a luciferase reporter. Exemplary cell lines include Jurkat T cells, although any suitable cell line known in the art may be used. For example, Jurkat cells expressing a luciferase reporter under the control of an NFAT promoter can be used as effector cells. Expression of luciferase by this cell line reflects TCR-mediated signaling.

The reporter cells can be transfected with each of the various fusion protein constructs, combinations of fusion protein constructs or controls described herein.

Expression of the fusion proteins in reporter cells can be confirmed by using fluorescently labeled MHC tetramers, for example Alexa Fluor 647-labeled MHC tetramer, to detect expression of the fusion protein.

To assay the activity of engineered receptors, target cells are loaded with antigen, or transfected with an RNA encoding an antigen, prior to exposure to the effector cells comprising the reporter and the engineered receptor. For example, target cells can be loaded with antigen at least 12, 14, 16, 18, 20, 22 or 24 hours prior to exposure to effector cells. Exemplary target cells include A375 cells, although any suitable cells known in the art may be used. The effector cells can then be co-cultured with target cells for a suitable period of time, for example 6 hours. Luciferase is then measured by luminescence reading after co-culture. Luciferase luminescence can be normalized to maximum and minimum intensity to allow comparison of activating peptide concentrations for each engineered receptor construct.

Provided herein are methods of determining the relative EC50 of engineered receptors of the disclosure. As used herein, “EC50” refers to the concentration of an inhibitor or agent where the response (or binding) is reduced by half EC50s of engineered receptors of the disclosure refer to concentration of antigen where binding of the engineered receptor to the antigen is reduced by half Binding of the antigen, or probe to the engineered receptor can be measured by staining with labeled peptide or labeled peptide-MHC complex. EC50 can be obtained by nonlinear regression curve fitting of reporter signal with peptide titration. Probe binding and EC50 can be normalized to the levels of benchmark TCR without a fusion protein.

### Polynucleotides and Vectors

The disclosure provides a polynucleotide system, comprising one or more polynucleotides comprising polynucleotide sequences encoding: an activator receptor comprising an extracellular antigen binding domain specific to an HLA-E antigen; and an inhibitory receptor comprising an extracellular antigen binding domain specific to a non-target antigen that is not expressed by the cancer cell due to a loss of heterozygosity in the cancer cell.

The disclosure provides vectors comprising the polynucleotide system described herein.

The disclosure provides polynucleotides encoding the sequence(s) of the activator and inhibitory receptors described herein. In some embodiments, the sequence of the activating and/or inhibitory receptor, or a fusion protein of the activator and/or inhibitory receptor is operably linked to a promoter. In some embodiments, the sequence encoding the activator receptor, or a polypeptide thereof, is operably linked to a first promoter, and the sequence encoding an inhibitory receptor, or a fusion protein thereof, is operably linked to a second promoter.

The disclosure provides vectors comprising the polynucleotides described herein.

The disclosure provides vectors encoding the coding sequence or sequences of any of the engineered receptors described herein. In some embodiments, the sequence of the activating and/or inhibitory receptor is operably linked to a promoter. In some embodiments, the sequence encoding the activating receptor is operably linked to a first promoter, and the sequence encoding an inhibitory receptor is operably linked to a second promoter.

In some embodiments, the activating receptor is encoded by a first vector and the inhibitory receptor is encoded by second vector. In some embodiments, both engineered receptors are encoded by a single vector.

In some embodiments, the first receptor is encoded by a first vector and the second receptor is encoded by a second vector. In some embodiments, both receptors are encoded by a single vector. In some embodiments, the first and/or second vector comprises an shRNA, for example a B2M shRNA.

In some embodiments, both receptors are encoded by a single vector. In some embodiments the vector comprises an shRNA, for example a B2M shRNA.

In some embodiments, the activator and inhibitory receptors are encoded by a single vector. Methods of encoding multiple polypeptides using a single vector will be known to persons of ordinary skill in the art, and include, inter alia, encoding multiple polypeptides under control of different promoters, or, if a single promoter is used to control transcription of multiple polypeptides, use of sequences encoding internal ribosome entry sites (IRES) and/or self-cleaving peptides. Exemplary self-cleaving peptides include T2A, P2A, E2A and F2A self-cleaving peptides. In some embodiments, the T2A self-cleaving peptide comprises a sequence of EGRGSLLTCGDVEENPGP (SEQ ID NO: 288). In some embodiments, the P2A self-cleaving peptide comprises a sequence of ATNFSLLKQAGDVEENPGP (SEQ ID NO: 196). In some embodiments, the E2A self-cleaving peptide comprises a sequence of QCTNYALLKLAGDVESNPGP (SEQ ID NO: 289). In some embodiments, the F2A self-cleaving peptide comprises a sequence of VKQTLNFDLLKLAGDVESNPGP (SEQ ID NO: 290). Any of the foregoing can also include an N terminal GSG linker. For example, a T2A self-cleaving peptide can also comprise a sequence of GSGEGRGSLLTCGDVEENPGP (SEQ ID NO: 492), which can be encoded by a sequence of

(SEQ ID NO: 493)
GGATCCGGAGAGGGCAGAGGCAGCCTGCTGACATGTGGCGACGTGGAAG
AGAACCCTGGCCCC.

In some embodiments, the vector is an expression vector, i.e. for the expression of the engineered receptors of the disclosure in a suitable cell.

Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

The expression of natural or synthetic nucleic acids encoding engineered receptors is typically achieved by operably linking a nucleic acid encoding the fusion protein or portions thereof to a promoter, and incorporating the construct into an expression vector. The vectors can be suitable for replication and integration eukaryotes. Typical cloning vectors contain transcription and translation terminators, initiation sequences, and promoters useful for regulation of the expression of the desired nucleic acid sequence.

The polynucleotides encoding the fusion proteins can be cloned into a number of types of vectors. For example, the polynucleotides can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to cells, such as immune cells, in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

Additional promoter elements, e.g., enhancers, regulate the frequency of transcriptional initiation. Typically, these are located in the region 30-110 basepairs (bp) upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In order to assess the expression of an engineered receptor, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected or transduced cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York). One method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

Regardless of the method used to introduce exogenous nucleic acids into a host cell or otherwise expose a cell to the inhibitor of the present invention, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

### Immune Cells

Provided herein are immune cells comprising the polynucleotides, vectors, fusion proteins and engineered receptors described herein.

The disclosure provides an immune cell responsive to loss of heterozygosity in a cancer cell, comprising: an activator receptor comprising an extracellular antigen binding domain specific to an HLA-E antigen; and an inhibitory receptor comprising an extracellular antigen binding domain specific to a non-target antigen that is not expressed by the cancer cell due to a loss of heterozygosity in the cancer cell. Alternatively, the inhibitory receptor comprises an extracellular antigen binding domain specific to a non-target antigen whose expression has been lost, or reduced below what is required for receptor activation, by the cancer cell through means other than loss of heterozygosity. For example, mutagenesis of the gene encoding the non-target antigen, or epigenetic modification can also lead to loss of non-target antigen expression in cancer cells.

In some embodiments, the non-target antigen is lost in the cancer cell through loss of heterozygosity or loss of expression. In some embodiments, the non-target antigen is an HLA class I allele or a minor histocompatibility antigen (MiHA). In some embodiments, the HLA class I allele comprises HLA-A, HLA-B, or HLA-C. In some embodiments, the HLA class I allele comprises HLA-A*02. In some embodiments, the HLA class I allele comprises HLA-A*11. In some embodiments, the cancer cell expresses HLA-E. In some embodiments, the cancer cell is a colorectal cancer cell, renal cancer cell, an ovarian cancer cell, a cervical cancer cell, a melanoma cancer cell, a urothelial cancer cell, a pancreatic cancer cell, a gastric cancer cell, a head and neck cancer cell, a lung cancer cell, a breast cancer cell or a lymphoma cell.

In some embodiments, the HLA-A*02 or HLA-A*11 non-target antigen is expressed by healthy cells of a subject. In some embodiments, the healthy cells of the subject express both the target antigen and the HLA-A*02 or HLA-A*11 non-target antigen. In some embodiments, the activator receptor and the inhibitory receptor together specifically activate the immune cell in the presence of the cancer cell.

In some embodiments, the immune cell is a T cell. In some embodiments, the T cell is a CD8+CD4− T cell. In some embodiments, the T cell is a CD8− CD4+ T cell. In some embodiments, the immune cell is autologous. In some embodiments, the immune cell is allogeneic.

As used herein, the term “immune cell” refers to a cell involved in the innate or adaptive (acquired) immune systems. Exemplary innate immune cells include phagocytic cells such as neutrophils, monocytes and macrophages, Natural Killer (NK) cells, polymophonuclear leukocytes such as neutrophils eosinophils and basophils and mononuclear cells such as monocytes, macrophages and mast cells. Immune cells with roles in acquired immunity include lymphocytes such as T-cells and B-cells.

As used herein, a “T-cell” refers to a type of lymphocyte that originates from a bone marrow precursor that develops in the thymus gland. There are several distinct types of T-cells which develop upon migration to the thymus, which include, helper CD4+ T-cells, cytotoxic CD8+ T cells, memory T cells, regulatory CD4+ T-cells and stem memory T-cells. Different types of T-cells can be distinguished by the ordinarily skilled artisan based on their expression of markers. Methods of distinguishing between T-cell types will be readily apparent to the ordinarily skilled artisan.

In some embodiments, the engineered immune cell expresses the first and second receptors at a ratio of about 100:1 to 1:100 of first receptor to second receptor. In some embodiments, the engineered immune cell expresses the first and second receptors at a ratio of about 50:1 to 1:50 of first receptor to second receptor. In some embodiments, the engineered immune cell expresses the first and second receptors at a ratio of about 10:1 to 1:10 of first receptor to second receptor. In some embodiments, the engineered immune cell expresses the first and second receptors at a ratio of about 5:1 to 1:5 of first receptor to second receptor. In some embodiments, the engineered immune cell expresses the first and second receptors at a ratio of about 3:1 to 1:3 of first receptor to second receptor. In some embodiments, the engineered immune cell expresses the first and second receptors at a ratio of about 2:1 to 1:2 of first receptor to second receptor. In some embodiments, the engineered immune cell expresses the first and second receptors at a ratio of about 1:1.

In some embodiments, the engineered immune cell comprising the engineered receptors of the disclosure is a T cell. In some embodiments, the T cell is an effector T cell or a regulatory T cell.

In some embodiments, the first receptor and the second receptor together specifically activate the immune cell in the presence of the target cell.

In some embodiments, the immune cell is selected form the group consisting of T cells, B cells and Natural Killer (NK) cells. In some embodiments, the immune cell is a gamma delta (T6) T cell. In some embodiments, the immune cell is an invariant T cell. In some embodiments, the immune cell is an invariant natural killer T cell (iNKT cell). In some embodiments, the immune cell is a T cell, an NK cell or a macrophage. In some embodiments, the immune cell is a B cell. In some embodiments, the immune cell is a Natural Killer (NK) cell. In some embodiments, the immune cell is CD8−. In some embodiments, the immune cell is CD8+. In some embodiments, the immune cell is CD4+. In some embodiments, the immune cell is CD4−. In some embodiments, the immune cell is CD8−/CD4+. In some embodiments, the immune cell is a CD8+/CD4− T cell.

In some embodiments, the immune cell is non-natural. In some embodiments, the immune cell is isolated.

Methods transforming populations of immune cells, such as T cells, with the vectors of the instant disclosure will be readily apparent to the person of ordinary skill in the art. For example, CD3+ T cells can be isolated from PBMCs using a CD3+ T cell negative isolation kit (Miltenyi), according to manufacturer's instructions. T cells can be cultured at a density of 1×10\hat{ }6 cells/mL in X-Vivo 15 media supplemented with 5% human A/B serum and 1% Pen/strep in the presence of CD3/28 Dynabeads (1:1 cell to bead ratio) and 300 Units/mL of IL-2 (Miltenyi). After 2 days, T cells can be transduced with viral vectors, such as lentiviral vectors using methods known in the art. In some embodiments, the viral vector is transduced at a multiplicity of infection (MOI) of 5. Cells can then be cultured in IL-2 or other cytokines such as combinations of IL-7/15/21 for an additional 5 days prior to enrichment. Methods of isolating and culturing other populations of immune cells, such as B cells, or other populations of T cells, will be readily apparent to the person of ordinary skill in the art. Although this method outlines a potential approach it should be noted that these methodologies are rapidly evolving. For example excellent viral transduction of peripheral blood mononuclear cells can be achieved after 5 days of growth to generate a >99% CD3+ highly transduced cell population.

Methods of activating and culturing populations of T cells comprising the engineered TCRs, CARs, fusion proteins or vectors encoding the fusion proteins of the instant disclosure, will be readily apparent to the person of ordinary skill in the art.

Whether prior to or after genetic modification of T cells to express an engineered TCR, the T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041, 10,040,846; and U.S. Pat. Appl. Pub. No. 2006/0121005.

In some embodiments, T cells of the instant disclosure are expanded and activated in vitro. Generally, the T cells of the instant disclosure are expanded in vitro by contact with a surface having attached thereto an agent that stimulates a CD3/TCR complex associated signal and an antigen that stimulates a co-stimulatory molecule on the surface of the T cells. In particular, T cell populations may be stimulated as described herein, such as by contact with an anti-CD3 antibody. For co-stimulation of an accessory molecule on the surface of the T cells, an antigen that binds the accessory molecule is used. For example, a population of T cells can be contacted with an anti-CD3 antibody and an anti-CD28 antibody, under conditions appropriate for stimulating proliferation of the T cells. To stimulate proliferation of either CD4+ T cells or CD8+ T cells, an anti-CD3 antibody and an anti-CD28 antibody can be used. Examples of an anti-CD28 antibody include 9.3, B-T3, XR-CD28 (Diaclone, Besangon, France) can be used as can other methods commonly known in the art (Berg et al., Transplant Proc. 30(8):3975-3977, 1998; Haanen et al., J. Exp. Med. 190(9):13191328, 1999; Garland et al., J. Immunol Meth. 227(1-2):53-63, 1999).

In some embodiments, the primary stimulatory signal and the co-stimulatory signal for the T cell may be provided by different protocols. For example, the agents providing each signal may be in solution or coupled to a surface. When coupled to a surface, the agents may be coupled to the same surface (i.e., in “cis” formation) or to separate surfaces (i.e., in “trans” formation). Alternatively, one agent may be coupled to a surface and the other agent in solution. In some embodiments, the agent providing the co-stimulatory signal is bound to a cell surface and the agent providing the primary activation signal is in solution or coupled to a surface. In certain embodiments, both agents can be in solution. In another embodiment, the agents may be in soluble form, and then cross-linked to a surface, such as a cell expressing Fc receptors or an antibody or other binding agent which will bind to the agents. In this regard, see for example, U.S. Patent Application Publication Nos. 20040101519 and 20060034810 for artificial antigen presenting cells (aAPCs) that are contemplated for use in activating and expanding T cells in the present invention.

In some embodiments, the two agents are immobilized on beads, either on the same bead, i.e., “cis,” or to separate beads, i.e., “trans.” By way of example, the agent providing the primary activation signal is an anti-CD3 antibody or an antigen-binding fragment thereof and the agent providing the co-stimulatory signal is an anti-CD28 antibody or antigen-binding fragment thereof, and both agents are co-immobilized to the same bead in equivalent molecular amounts. In one embodiment, a 1:1 ratio of each antibody bound to the beads for CD4+ T cell expansion and T cell growth is used. In some embodiments, the ratio of CD3:CD28 antibody bound to the beads ranges from 100:1 to 1:100 and all integer values there between. In one aspect of the present invention, more anti-CD28 antibody is bound to the particles than anti-CD3 antibody, i.e., the ratio of CD3:CD28 is less than one. In certain embodiments of the invention, the ratio of anti CD28 antibody to anti CD3 antibody bound to the beads is greater than 2:1.

Ratios of particles to cells from 1:500 to 500:1 and any integer values in between may be used to stimulate T cells or other target cells. As those of ordinary skill in the art can readily appreciate, the ratio of particles to cells may depend on particle size relative to the target cell. For example, small sized beads could only bind a few cells, while larger beads could bind many. In certain embodiments the ratio of cells to particles ranges from 1:100 to 100:1 and any integer values in-between and in further embodiments the ratio comprises 1:9 to 9:1 and any integer values in between, can also be used to stimulate T cells. In some embodiments, a ratio of 1:1 cells to beads is used. One of skill in the art will appreciate that a variety of other ratios may be suitable for use in the present invention. In particular, ratios will vary depending on particle size and on cell size and type.

In further embodiments of the present invention, the cells, such as T cells, are combined with agent-coated beads, the beads and the cells are subsequently separated, and then the cells are cultured. In an alternative embodiment, prior to culture, the agent-coated beads and cells are not separated but are cultured together. In a further embodiment, the beads and cells are first concentrated by application of a force, such as a magnetic force, resulting in increased ligation of cell surface markers, thereby inducing cell stimulation.

By way of example, cell surface proteins may be ligated by allowing paramagnetic beads to which anti-CD3 and anti-CD28 are attached to contact the T cells. In one embodiment the cells (for example, CD4+ T cells) and beads (for example, DYNABEADS CD3/CD28 T paramagnetic beads at a ratio of 1:1) are combined in a buffer. Again, those of ordinary skill in the art can readily appreciate any cell concentration may be used. In certain embodiments, it may be desirable to significantly decrease the volume in which particles and cells are mixed together (i.e., increase the concentration of cells), to ensure maximum contact of cells and particles. For example, in one embodiment, a concentration of about 2 billion cells/ml is used. In another embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. In some embodiments, cells that are cultured at a density of 1×10⁶ cells/mL are used.

In some embodiments, the mixture may be cultured for several hours (about 3 hours) to about 14 days or any hourly integer value in between. In another embodiment, the beads and T cells are cultured together for 2-3 days. Conditions appropriate for T cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-7, IL-4, IL-7, GM-CSF, IL-10, IL-12, IL-15, TGFβ, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. In some embodiments, the media comprises X-VIVO-15 media supplemented with 5% human A/B serum, 1% penicillin/streptomycin (pen/strep) and 300 Units/ml of IL-2 (Miltenyi).

The T cells are maintained under conditions necessary to support growth, for example, an appropriate temperature (e.g., 37° C.) and atmosphere (e.g., air plus 5% CO2).

In some embodiments, the T cells comprising engineered TCRs of the disclosure are autologous. Prior to expansion and genetic modification, a source of T cells is obtained from a subject. Immune cells such as T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain embodiments of the present invention, any number of T cell lines available in the art, may be used. In certain embodiments of the present invention, T cells can be obtained from a unit of blood collected from a subject using any number of techniques known to the skilled artisan, such as Ficoll™ separation.

In some embodiments, cells from the circulating blood of an individual are obtained by apheresis. The apheresis product typically contains lymphocytes, including T cells, monocytes, granulocytes, B cells, other nucleated white blood cells, red blood cells, and platelets. In some embodiments, the cells collected by apheresis may be washed to remove the plasma fraction and to place the cells in an appropriate buffer or media for subsequent processing steps. In some embodiments, the cells are washed with phosphate buffered saline (PBS). In alternative embodiments, the wash solution lacks calcium and may lack magnesium or may lack many if not all divalent cations. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca2±free, Mg2±free PBS, PlasmaLyte A, or other saline solution with or without buffer. Alternatively, the undesirable components of the apheresis sample may be removed and the cells directly resuspended in culture media.

In some embodiments, immune cells such as T cells are isolated from peripheral blood lymphocytes by lysing the red blood cells and depleting the monocytes, for example, by centrifugation through a PERCOLL™ gradient or by counterflow centrifugal elutriation. Specific subpopulations of immune cells, such as T cells, B cells, or CD4+ T cells can be further isolated by positive or negative selection techniques. For example, in one embodiment, T cells are isolated by incubation with anti-CD4-conjugated beads, for a time period sufficient for positive selection of the desired T cells.

Enrichment of an immune cell population, such as a T cell population, by negative selection can be accomplished with a combination of antibodies directed to surface markers unique to the negatively selected cells. One method is cell sorting and/or selection via negative magnetic immune-adherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD 14, CD20, CD 11b, CD 16, HLA-DR, and CD8.

For isolation of a desired population of immune cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads.

In some embodiments, the cells may be incubated on a rotator for varying lengths of time at varying speeds at either 2-10° C. or at room temperature.

T cells for stimulation, or PBMCs from which immune cells such as T cells are isolated, can also be frozen after a washing step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

The disclosure provides an immune cell expressing the activator and/or inhibitory receptors described herein, wherein the immune cell has reduced expression and/or function the major histocompatibility (MHC) class I complex.

In some embodiments, the immune cell is autologous. For example, the immune cells is isolated or derived from same subject who will receive the cell as part of a therapeutic regimen. It can be advantageous to modify autologous immune cells to have reduced expression and/or function of MHC class I with the inhibitor receptor is specific to an MHC class I antigen. Without wishing to be bound by theory, modification of autologous immune cells to have reduced expression and/or function of MHC class I reduces binding of the inhibitor receptor by MHC class I expressed by the immune cells, either in cis or in trans.

In some embodiments, the immune cell is all allogeneic. Allogeneic immune cells can be derived from a donor other than the subject to which the immune cells will be administered. Allogeneic immune cells have been commonly referred to in cell therapy as “off-the-shelf” or “universal” because of the possibility for allogeneic cells to be prepared and stored for use in subjects of a variety of genotypes.

Any suitable methods of reducing expression and/or function the MHC class I complex are envisaged as within the scope of the instant disclosure, and include, inter alia, expression of interfering RNAs that knock down one or more RNAs encoding MHC class I components, or modifications of genes encoding MHC class I components. Methods of reducing expression and/or function of the MHC class I complex described herein are suitable for use with both allogeneic and autologous immune cells.

The major histocompatibility complex (MHC) is a locus on the vertebrate genome that encodes a set of polypeptides required for the adaptive immune system. Among these are MHC class I polypeptides that include HLA-A, HLA-B, and HLA-C and alleles thereof. MHC class I alleles are highly polymorphic and expressed in all nucleated cells. MHC class I polypeptides encoded by HLA-A, HLA-B, and HLA-C and alleles thereof form heterodimers with β2 microglobulin (B2M) and present in complex with antigens on the surface of cells. As referred to herein, an MHC class I gene or polypeptide may refer to any polypeptide found in the MHC or the corresponding gene encoding said polypeptide. In some embodiments, the immune cells of the disclosure are inactivated by an inhibitor ligand comprising an MHC class I polypeptide, e.g. HLA-A, HLA-B, and HLA-C and alleles thereof. HLA-A alleles can be, for example and without limitation, HLA-A*02, HLA-A*02:01, HLA-A*02:01:01, HLA-A*02:01:01:01, and/or any gene that encodes protein identical or similar to HLA-A*02 protein. Thus, to prevent autocrine signaling/binding as described herein, it is desirable to eliminate or reduce expression of polypeptides encoded by HLA-A, HLA-B, and HLA-C and alleles thereof in the immune cells.

Immune Cells with Reduced MHC Class I Polypeptide Expression

In some embodiments, the immune cells described herein are modified to inactivate, or reduce or eliminate expression or function of an endogenous gene encoding an allele of an endogenous MHC class I polypeptide. In some embodiments, the gene encoding the MHC class I polypeptide is HLA-A, HLA-B, and/or HLA-C. HLA-A, HLA-B and HLA-C are encoded by the HLA-A, HLA-B and HLA-C loci. Each of HLA-A, HLA-B and HLA-C includes many variant alleles, all of which are envisaged as within the scope of the instant disclosure. In some embodiments, the gene encoding the MHC class I polypeptide is HLA-A. In some embodiments, the gene encoding the MHC class I polypeptide is HLA-A*02. In some embodiments, the gene encoding the MHC class I polypeptide is HLA-A*02:01. In some embodiments, the gene encoding the MHC class I polypeptide is HLA-A*02:01:01. In some embodiments, the gene encoding the MHC class I polypeptide is HLA-A*02:01:01:01.

In some embodiments, the genetically engineered immune cells described herein are modified to reduce or eliminate expression of the B2M gene product. The beta-2 microglobulin (B2M) gene encodes a protein that associates with the major histocompatibility complex (MHC) class I, i.e. MHC-I complex. The MHC-I complex is required for presentation of antigens on the cell surface. The MHC-I complex is disrupted and non-functional when the B2M is deleted (Wang D et al. Stem Cells Transl Med. 4:1234-1245 (2015)). Furthermore, the B2M gene can be disrupted with high efficiency using gene editing techniques known in the art (Ren et al. Clin. Cancer Res. 23:2255-2266 (2017)). Reducing or eliminating B2M can reduce, or eliminate functional MHC I on the surface of the immune cell.

The disclosure provides gene editing systems for editing an endogenous target gene in an immune cell. The disclosure provides interfering RNAs specific to sequences of target genes. Gene editing systems such as CRISPR/Cas systems, TALENs and zinc fingers can be used to generate double strand breaks, which, through gene repair mechanisms such as homology directed repair or non-homologous end joining (NHEJ), can be used to introduce mutations. NHEJ after resection of the ends of the break, or improper end joining, can be used to introduce deletions. In some embodiments, the target gene comprises a gene encoding a subunit of the MHC-I complex.

In some embodiments, modifying the gene encoding the MHC class I polypeptide comprises deleting all or a portion of the gene. In some embodiments, modifying the gene encoding the MHC class I polypeptide comprises introducing a mutation in the gene. In some embodiments, the mutation comprises a deletion, insertion, substitution, or frameshift mutation. In some embodiments, modifying the gene comprises using a nucleic acid guided endonuclease.

Gene sequences for the target genes described herein are known in the art. The sequences can be found at public databases, such as NCBI GenBank or the NCBI nucleotide database. Sequences may be found using gene identifiers, for example, the HLA-A gene has NCBI Gene ID: 3105, the HLA-B gene has NCBI Gene ID: 3106, the HLA-C gene has NCBI Gene ID: 3107, and the B2M gene has NCBI Gene ID: 567 and NCBI Reference Sequence: NC_000015.10. Gene sequences may also be found by searching public databases using keywords. For example, HLA-A alleles may be found in the NCBI nucleotide database by searching keywords, “HLA-A*02”, “HLA-A*02:01”, “HLA-A*02:01:01”, or “HLA-A*02:01:01:01.” These sequences can be used for targeting in various gene editing techniques known in the art. Table 8 provides non-limiting illustrative sequences of HLA-A allele and B2M gene sequences targeted for modification as described herein.

TABLE 8
Exemplary Target Gene Sequences

| | |
|---|---|
| B2M mRNA | (SEQ ID NO: 494) |
| B2M Gene (GenBank: 567) | (SEQ ID NO: 495) |
| HLA-A*02:01:01:01 sequence encoding mRNA | (SEQ ID NO: 496) |
| HLA-A*02 (GenBank: LK021978.1) | (SEQ ID NO: 497) |

The person of ordinary skill in the art will appreciate that T can be substituted for U to convert an RNA sequence to a DNA sequence and vice versa, and both are envisaged as target gene sequences of the disclosure.

In some embodiments, a target gene is edited in the immune cells described herein using a nucleic acid guided endonuclease. Exemplary nucleic acid guided endonucleases include Class II endonucleases, such as CRISPR/Cas9.

“CRISPR” or “CRISPR gene editing” as used herein refers to a set of clustered regularly interspaced short palindromic repeats, or a system comprising such a set of repeats. “Cas”, as used herein, refers to a CRISPR-associated protein. A “CRISPR/Cas” system refers to a system derived from CRISPR and Cas which can be used to silence, knock out, or mutate a target gene.

CRISPR/Cas systems are classified by class and by type. Class 2 systems currently represent a single interference protein that is categorized into three distinct types (types II, V and VI). Any class 2 CRISPR/Cas system suitable for gene editing, for example a type II, a type V or a type VI system, is envisaged as within the scope of the instant disclosure.

The CRISPR sequence, sometimes called a CRISPR locus, comprises alternating repeats and spacers. In a naturally-occurring CRISPR, the spacers usually comprise sequences foreign to the bacterium such as a plasmid or phage sequence. As described herein, spacer sequences may also be referred to as “targeting sequences.” In CRISPR/Cas systems for a genetic engineering, the spacers are derived from the target gene sequence (the gNA).

An exemplary Class 2 type II CRISPR system relies on the protein Cas9, which is a nuclease with two active cutting sites, one for each strand of the double helix. Combining Cas9 and modified CRISPR locus RNA can be used in a system for gene editing. Pennisi (2013) Science 341: 833-836. In some embodiments, the Cas protein used to modify the immune cells is Cas9.

The CRISPR/Cas system can thus be used to edit a target gene, such as a gene targeted for editing in the immune cells described herein, by adding or deleting a base pair, or introducing a premature stop which thus decreases expression of the target. The CRISPR/Cas system can alternatively be used like RNA interference, turning off a target gene in a reversible fashion. In a mammalian cell, for example, the RNA can guide the Cas protein to a target gene promoter, sterically blocking RNA polymerases.

A Cas protein may be derived from any bacterial or archaeal Cas protein. Any suitable CRISPR/Cas system is envisaged as within the scope of the instant disclosure.

Artificial CRISPR/Cas systems can be generated which inhibit a target gene, using technology known in the art, e.g., that described in U.S. Publication No. 20140068797, and Cong (2013) Science 339: 819-823. Other artificial CRISPR/Cas systems that are known in the art may also be generated which inhibit a target gene, e.g., that described in Tsai (2014) Nature Biotechnol., 32:6 569-576, U.S. Pat. Nos. 8,871,445; 8,865,406; 8,795,965; 8,771,945; and 8,697,359. Methods of designing suitable gNAs for a particular Cas protein will be known by persons of ordinary skill in the art.

The present disclosure provides gene-targeting guide nucleic acids (gNAs) that can direct the activities of an associated polypeptide (e.g., nucleic acid guided endonuclease) to a specific target gene sequence within a target nucleic acid genome. The genome-targeting nucleic acid can be an RNA. A genome-targeting RNA is referred to as a “guide RNA” or “gRNA” herein. A guide RNA can comprise at least a targeting sequence that hybridizes to a target nucleic acid sequence of interest, and a CRISPR repeat sequence. In some Type II systems, the gRNA also comprises a second RNA called the tracrRNA sequence, also referred to herein as a “scaffold” sequence. In the Type II guide RNA (gRNA), the CRISPR repeat sequence and scaffold sequence hybridize to each other to form a duplex. In the Type V guide RNA (gRNA), the crRNA forms a duplex. In both systems, the duplex can bind a site-directed polypeptide, such that the guide RNA and site-directed polypeptide form a complex. The gene-targeting nucleic acid can provide target specificity to the complex by virtue of its association with the site-directed polypeptide. The gene-targeting nucleic acid thus can direct the activity of the site-directed polypeptide.

In some embodiments, the disclosure provides a guide RNA comprising a targeting sequence and a guide RNA scaffold sequence, wherein the targeting sequence is complementary to the sequence of a target gene.

Exemplary guide RNAs include targeting sequences of about 15-20 bases. As is understood by the person of ordinary skill in the art, each gRNA can be designed to include a targeting sequence complementary to its genomic target sequence.

The gene targeting nucleic acid can be a double-molecule guide RNA. The gene targeting nucleic acid can be a single-molecule guide RNA. The gene targeting nucleic acid can be any known configuration of guide RNA known in the art, such as, for example, including paired gRNA, or multiple gRNAs used in a single step.

A double-molecule guide RNA can comprise two strands of RNA. The first strand comprises a sequence in the 5′ to 3′ direction, an optional spacer extension sequence, a targeting sequence and a minimum CRISPR repeat sequence. The second strand can comprise a minimum tracrRNA sequence (complementary to the minimum CRISPR repeat sequence), a 3′ tracrRNA sequence and an optional tracrRNA extension sequence.

In some embodiments, guide RNA or single-molecule guide RNA (sgRNA) can comprise a targeting sequence and a scaffold sequence. In some embodiments, the scaffold sequence is a Cas9 gRNA sequence. In some embodiments, the scaffold sequence is encoded by a DNA sequence that comprises a sequence that shares at least 90%, at least 95%, at least 96%, at least 97%, at least 98%, or at least 99% identity to

(SEQ ID NO: 498)
GTTTTAGAGCTAGAAATAGCAAGTTAAAATAAGGCTAGTCCGTTATCAA
CTTGAAAAAGTGGCACCGAGTCGGTGCTTTTTTT.

In some embodiments, the scaffold sequence is encoded by a DNA sequence that comprises

(SEQ ID NO: 498)
GTTTTAGAGCTAGAAATAGCAAGTTAAAATAAGGCTAGTCCGTTATCAA
CTTGAAAAAGTGGCACCGAGTCGGTGCTTTTTTT.

In some embodiments, for example those embodiments where the CRISPR/Cas system is a Cas9 system, the sgRNA can comprise a 20 nucleotide targeting sequence at the 5′ end of the sgRNA sequence. The sgRNA can comprise a less than a 20 nucleotide targeting sequence at the 5′ end of the sgRNA sequence. The sgRNA can comprise a more than 20 nucleotide targeting sequence at the 5′ end of the sgRNA sequence. The sgRNA can comprise a variable length targeting sequence with 17-30 nucleotides at the 5′ end of the sgRNA sequence.

Suitable scaffold sequences, and arrangement of scaffold targeting sequences, will depend on choice of endonuclease, and will be known to persons of skill in the art.

A single-molecule guide RNA (sgRNA) in a Type II system, e.g. Cas9, can comprise, in the 5′ to 3′ direction, a minimum CRISPR repeat sequence and a targeting sequence.

The targeting sequence of a gRNA hybridizes to a sequence in a target nucleic acid of interest. The targeting sequence of a genome-targeting nucleic acid can interact with a target nucleic acid in a sequence-specific manner via hybridization (i.e., base pairing). The nucleotide sequence of the targeting sequence can vary depending on the sequence of the target nucleic acid of interest.

In a Cas9 system described herein, the targeting sequence can be designed to hybridize to a target nucleic acid that is located 5′ of the reverse complement of a PAM of the Cas9 enzyme used in the system. The targeting sequence may perfectly match the target sequence or may have mismatches. Each CRISPR/Cas system protein may have a particular PAM sequence, in a particular orientation and position, so that it recognizes in a target DNA. For example, S. pyogenes Cas9 recognizes in a target nucleic acid a PAM that comprises the sequence 5′-NRG-3′, where R comprises either A or G, where N is any nucleotide and N is immediately 3′ of the target nucleic acid sequence targeted by the targeting sequence. Selection of appropriate PAM sequences will be apparent to the person of ordinary skill in the art.

The target sequence is complementary to, and hybridizes with, the targeting sequence of the gRNA. The target nucleic acid sequence can comprise 20 nucleotides. The target nucleic acid can comprise less than 20 nucleotides. The target nucleic acid can comprise more than 20 nucleotides. The target nucleic acid can comprise at least: 5, 10, 15, 16, 17, 18, 19, 20, 21, 22, 23, 24, 25, 30 or more nucleotides. In some embodiments, for example those embodiments where the CRISPR/Cas system is a Cas9 system, the target nucleic acid sequence can comprise 20 nucleotides immediately 5′ of the first nucleotide of the reverse complement of the PAM sequence. This target nucleic acid sequence is often referred to as the PAM strand or a target strand, and the complementary nucleic acid sequence is often referred to the non-PAM strand or non-target strand. One of skill in the art would recognize that the targeting sequence hybridizes to the non-PAM strand of the target nucleic acid, see e.g., US20190185849A1.

The targeting sequence can be designed or chosen using computer programs known to persons of ordinary skill in the art. Available computer programs can take as input NCBI gene IDs, official gene symbols, Ensembl Gene IDs, genomic coordinates, or DNA sequences, and create an output file containing sgRNAs targeting the appropriate genomic regions designated as input (e.g., Doench et al. Nat Biotechnol. 34:184-191 (2016)). The disclosure provides guide RNAs comprising a targeting sequence. In some embodiments, the guide RNA further comprises a guide RNA scaffold sequence. In some embodiments, the targeting sequence is complementary to the sequence of a target gene selected from the group consisting of HLA-A, HLA-B, HLA-C, B2M or an allele thereof. In some embodiments, the target gene is an HLA-A gene. In some embodiments, the target gene is an HLA-B gene. In some embodiments, the target gene is an HLA-C gene. In some embodiments the target gene is HLA-A, HLA-B, HLA-C, or a combination thereof. In some embodiments, targeting sequence comprises a sequence that shares about 90%, about 95%, about 96%, about 97%, about 98%, about 99% identity to or is identical to a sequence disclosed in Table 10.

In some embodiments, the gNAs specifically target a sequence of HLA-A*02 alleles. For example, the gRNAs specifically target, and hybridize to, a sequence shared by all HLA-A*02 alleles, but that is not shared by HLA-A*02 and HLA-A*03 alleles. In some embodiments, the gNAs specifically target a sequence of HLA-A*02:01 alleles. In some embodiments, the gNAs specifically target a sequence of HLA-A*02:01:01 alleles. In some embodiments, the gNAs specifically target a sequence of HLA-A*02:01:01:01 alleles.

In some embodiments, the gNAs specifically target a coding DNA sequence of HLA-A*02.

In some embodiments, the gNAs specifically target a coding DNA sequence that is shared by more than 1000 HLA-A*02 alleles. In some embodiments, the gNAs that specifically target a coding DNA sequence in greater than 1000 HLA-A*02 alleles comprise a sequence that shares about 90%, about 95%, about 96%, about 97%, about 98%, about 99% identity or is identical to a sequence selected from SEQ ID NOs: 499-618.

The sequences in SEQ ID NOs: 499-618 are presented as DNA sequences. The skilled artisan will understand that thymine (T) can be replaced with uracil (U) in any DNA sequence including those set forth in SEQ ID NOs: 499-618, to arrive at the corresponding RNA sequence.

The sequences disclosed in SEQ ID NOs: 499-618 include the corresponding genomic sequences, inclusive of the PAM sequence (NGG), with the exception of SEQ ID NOS: 499-504. The skilled artisan will understand that the targeting sequence of the gRNA does not include three 3′ terminal nucleotides of the sequences in SEQ ID NOs: 499-618, which represent the corresponding PAM site for the gRNA.

The disclosure provides gNAs comprising a targeting sequence specific to the B2M gene. In some embodiments, the gNAs specifically target the coding sequence (CDS) sequence of the B2M gene. In some embodiments, the gNA comprises a sequence that targets the B2M gene promoter sequence.

In some embodiments the gNA comprise a targeting sequence and a gNA scaffold sequence. In some embodiments, the targeting sequence comprises a sequence set forth in SEQ ID NOs: 619-732, or a sequence shares about 90%, about 95%, about 96%, about 97%, about 98%, about 99% identity thereto.

In some embodiments, the targeting sequence is complementary to a sequence of the B2M gene. In some embodiments, the B2M gene comprises a sequence that shares about 90%, about 95%, about 96%, about 97%, about 98%, about 99% identity to the B2M sequence set forth in Table 8.

In some embodiments, the immune cells described herein are edited using TALEN gene editing.

“TALEN” or “TALEN gene editing” refers to a transcription activator-like effector nuclease, which is an artificial nuclease used to edit a target gene.

In some embodiments, a target gene is edited in the immune cells described herein using ZFN gene editing.

“ZFN” or “Zinc Finger Nuclease” or “ZFN gene editing” refer to a zinc finger nuclease, an artificial nuclease which can be used to edit a target gene.

Like a TALEN, a ZFN must dimerize to cleave DNA. Thus, a pair of ZFNs are required to target non-palindromic DNA sites. The two individual ZFNs must bind opposite strands of the DNA with their nucleases properly spaced apart.

ZFNs specific to sequences in a target gene can be constructed using any method known in the art.

In some embodiments, the expression and of function of one or more MCH-I components are reduced using RNA interference. “RNAi” or “RNA interference” refers to the process of sequence-specific post-transcriptional gene silencing, mediated by double-stranded RNA (dsRNA). Duplex RNAs such as siRNA (small interfering RNA), miRNA (micro RNA), shRNA (short hairpin RNA), ddRNA (DNA-directed RNA), piRNA (Piwi-interacting RNA), or rasiRNA (repeat associated siRNA) and modified forms thereof are all capable of mediating RNA interference. These dsRNA molecules may be commercially available or may be designed and prepared based on known sequence information. The anti-sense strand of these molecules can include RNA, DNA, PNA, or a combination thereof. DNA/RNA chimeric polynucleotides include, but are not limited to, a double-strand polynucleotide composed of DNA and RNA that inhibits the expression of a target gene. dsRNA molecules can also include one or more modified nucleotides, as described herein, which can be incorporated on either or both strands.

As used herein with respect to RNA interference, “target gene” or “target sequence” refers to a gene or gene sequence whose corresponding RNA is targeted for degradation through the RNAi pathway using dsRNAs or siRNAs as described herein. Exemplary target gene sequences are shown in Table 8. To target a gene, for example using an siRNA, the siRNA comprises an anti-sense region complementary to, or substantially complementary to, at least a portion of the target gene or sequence, and sense strand complementary to the anti-sense strand. The double stranded RNA molecule of the disclosure may be in the form of any type of RNA interference molecule known in the art. In some embodiments, the double stranded RNA molecule is a small interfering RNA (siRNA). In other embodiments, the double stranded RNA molecule is a short hairpin RNA (shRNA) molecule. In other embodiments, the double stranded RNA molecule is a Dicer substrate that is processed in a cell to produce an siRNA. In other embodiments the double stranded RNA molecule is part of a microRNA precursor molecule.

In some embodiments, the shRNA is a length to be suitable as a Dicer substrate, which can be processed to produce a RISC active siRNA molecule. See, e.g., Rossi et al., US2005/0244858.

A Dicer substrate double stranded RNA (e.g. a shRNA) can be of a length sufficient that it is processed by Dicer to produce an active siRNA, and may further include one or more of the following properties: (i) the Dicer substrate shRNA can be asymmetric, for example, having a 3′ overhang on the anti-sense strand, (ii) the Dicer substrate shRNA can have a modified 3′ end on the sense strand to direct orientation of Dicer binding and processing of the dsRNA to an active siRNA, for example the incorporation of one or more DNA nucleotides, and (iii) the first and second strands of the Dicer substrate ds RNA can be from 21-30 bp in length.

In some embodiments, the interfering RNAs comprise a sequence complementary to a sequence of a B2M mRNA. In some embodiments, the interfering RNA is capable of inducing RNAi-mediated degradation of the B2M mRNA. In some embodiments, the B2M mRNA sequence comprises a coding sequence. In some embodiments, the B2M mRNA sequence comprises an untranslated region.

In some embodiments, the interfering RNAs comprise a sequence complementary to a sequence of an HLA-A*02 mRNA. In some embodiments, the interfering RNA is capable of inducing RNAi-mediated degradation of the HLA-A*02 mRNA. In some embodiments, the HLA-A*02 mRNA sequence comprises a coding sequence. In some embodiments, the HLA-A*02 mRNA sequence comprises an untranslated region.

In some embodiments, the interfering RNAs comprise a sequence complementary to a sequence of an HLA-A*03 mRNA. In some embodiments, the interfering RNA is capable of inducing RNAi-mediated degradation of the HLA-A*03 mRNA. In some embodiments, the HLA-A*03 mRNA sequence comprises a coding sequence. In some embodiments, the HLA-A*03 mRNA sequence comprises an untranslated region.

In some embodiments, the interfering RNA is a short hairpin RNA (shRNA). In some embodiments, the shRNA comprises a first sequence, having from 5′ to 3′ end a sequence complementary to the B2M mRNA; and a second sequence, having from 5′ to 3′ end a sequence complementary to the first sequence, wherein the first sequence and second sequence form the shRNA.

In some embodiments, the first sequence is 18, 19, 20, 21, or 22 nucleotides. In some embodiments, the first sequence is complementary to a sequence selected from the sequences set forth in Tables 9 and 10. In some embodiments, the first sequence has GC content greater than or equal to 25% and less than 60%. In some embodiments, the first sequence is complementary to a sequence selected from the sequences set forth in Tables 9 and 10. In some embodiments, the first sequence does not comprise four nucleotides of the same base or a run of seven C or G nucleotide bases. In some embodiments, the first sequence is 21 nucleotides.

In some cases, the first sequence may have 100% identity, i.e. complete identity, homology, complementarity to the target nucleic acid sequence. In other cases, there may be one or more mismatches between the first sequence and the target nucleic acid sequence. For example, there may be 1, 2, 3, 4, 5, 6, or 7 mismatches between the sense region and the target nucleic acid sequence.

An exemplary sequence encoding a B2M shRNA comprises a sequence of GCACTCAAAGCTTGTTAAGATCGAAATCTTAACAAGCTTTGAGTGC (SEQ ID NO: 871), or a sequence having at least 90%, at least 95%, at least 97% or at least 99% identity thereto. A further exemplary sequence encoding a B2M shRNA comprises a sequence of GTTAACTTCCAATTTACATACCGAAGTATGTAAATTGGAAGTTAAC (SEQ ID NO: 872), or a sequence having at least 90%, at least 95%, at least 97% or at least 99% identity thereto.

In some embodiments, the interfering RNAs comprise a sequence complementary to a sequence of an HLA-A*02 mRNA. In some embodiments, the interfering RNA is capable of inducing RNAi-mediated degradation of the HLA-A*02 mRNA. In some embodiments, the HLA-A*02 mRNA sequence comprises a coding sequence. In some embodiments, the HLA-A*02 mRNA sequence comprises an untranslated region.

In some embodiments, the interfering RNA is a short hairpin RNA (shRNA). In some embodiments, the shRNA comprises a first sequence, having from 5′ to 3′ end a sequence complementary to the HLA-A*02 mRNA; and a second sequence, having from 5′ to 3′ end a sequence complementary to the first sequence, wherein the first sequence and second sequence form the shRNA.

In some embodiments, the first sequence and second sequence are separated by a linker, sometimes referred to as a loop. In some embodiments, both the first sequence and the second sequence are encoded by one single-stranded RNA or DNA vector. In some embodiments, the loop is between the first and second sequences. In these embodiments, and the first sequence and the second sequence hybridize to form a duplex region. The first sequence and second sequence are joined by a linker sequence, forming a “hairpin” or “stem-loop” structure. The shRNA can have complementary first sequences and second sequences at opposing ends of a single stranded molecule, so that the molecule can form a duplex region with the complementary sequence portions, and the strands are linked at one end of the duplex region by a linker (i.e. loop sequence). The linker, or loop sequence, can be either a nucleotide or non-nucleotide linker. The linker can interact with the first sequence, and optionally, second sequence through covalent bonds or non-covalent interactions.

Any suitable nucleotide loop sequence is envisaged as within the scope of the disclosure. An shRNA of this disclosure may include a nucleotide, non-nucleotide, or mixed nucleotide/non-nucleotide linker that joins the first sequence of the shRNA to the second sequence of the shRNA. A nucleotide loop sequence can be >2 nucleotides in length, for example about 4, 5, 6, 7, 8, 9, 10, 11, 12, 13, 14, or 15 nucleotides in length. Illustrative loop sequences are disclosed in Table 10.

In some embodiments, the shRNA further comprises a 5′ flank sequence and a 3′ flank sequence. In some embodiments, wherein the 5′ flank sequence is joined to the 5′ end of the first sequence, and wherein the 3′ flank sequence is joined to the 3′ end of the second sequence.

Without wishing to be bound by theory, it is thought that flanking shRNA stem loop sequence with 5′ and 3′ sequences similar to those found in microRNAs can target the shRNA for processing by the endogenous microRNA processing machinery, increasing the effectiveness of shRNA processing. Alternatively, or in addition, flanking sequences may increase shRNA compatibility with polymerase II or polymerase III promoters, leading to more effective regulation of shRNA expression.

In some embodiments, the 5′ flank sequence is selected from the sequences set forth in Table 9. Illustrative flank sequence are shown in Table 9.

TABLE 9
Illustrative flank sequences

| SEQ ID NO | 5′ Flank Sequence |
|---|---|
| NA | GG |
| 959 | ACACCAUGUUGCCAGUCUCUAGG |
| 960 | UGAUAGCAAUGUCAGCAGUGCCU |
| 961 | UAUUGCUGUUGACAGUGAGCGAC |
| SEQ ID NO | 3′ Flank Sequence |
| 962 | UGGCGUCUGGCCCAACCACAC |
| 963 | GUAAGGUUGACCAUACUCUAC |

In some embodiments, the first and second sequence are present on a single stranded polynucleotide, wherein the first sequence and second sequence are separated by 4, 5, 6, 7, 8, 9, 10, 11, 12, 13, 14, or 15 nucleotides, wherein the 4, 5, 6, 7, 8, 9, 10, 11, 12, 13, 14, or 15 nucleotides form a loop region in the shRNA. In some embodiments, the loop region comprises a sequence selected from the sequences set forth in Table 10.

TABLE 10
Illustrative loop region sequences

| SEQ ID NO | Loop Region Sequence |
|---|---|
| 22001 | CGAA |
| 22002 | UUCAAGA |
| 22003 | AUAUUCA |
| 22004 | UGUGCUGUC |
| 22005 | CUCGAG |
| 964 | CUUCCUGUCAGA |
| 965 | CUUCCCUUUGUCAGA |
| 966 | GUGUUAUUCUUG |
| 967 | GUGUCUUAAUUG |
| 968 | GUGUUAGUCUUG |
| 969 | UCAAGAG |
| 970 | GGACAUCCAGGG |
| 971 | GUGAAGCCACAGAUG |
| 972 | GAUUCUAAAA |

shRNAs of the disclosure may be generated exogenously by chemical synthesis, by in vitro transcription, or by cleavage of longer double-stranded RNA with Dicer or another appropriate nuclease with similar activity. Chemically synthesized siRNAs, produced from protected ribonucleoside phosphoramidites using a conventional DNA/RNA synthesizer, may be obtained from commercial suppliers such as Millipore Sigma (Houston, Tex.), Ambion Inc. (Austin, Tex.). Invitrogen (Carlsbad, Calif.), or Dharmacon (Lafayette, Colo.). siRNAs can be purified by extraction with a solvent or resin, precipitation, electrophoresis, chromatography, or a combination thereof, for example. Alternatively, siRNAs may be used with little if any purification to avoid losses due to sample processing.

In some embodiments, shRNAs of the disclosure can be produced using an expression vector into which a nucleic acid encoding the double stranded RNA has been cloned, for example under control of a suitable promoter.

### Pharmaceutical Compositions

The disclosure provides pharmaceutical compositions comprising immune cells expressing the engineered receptors of the disclosure and a pharmaceutically acceptable diluent, carrier or excipient. In some embodiments, the pharmaceutical composition is for use as a medicament in the treatment of cancer. In some embodiments, the cancer is breast cancer, bladder cancer, ovarian cancer, gastric cancer, salivary duct carcinoma, non-small cell lung cancer, pancreatic cancer, or colon cancer. In some embodiments, the cancer is breast cancer. In some embodiments, the cancer is gastric cancer.

Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; and preservatives.

### Methods

Provided herein are methods of selectively killing an HLA-E positive cell exhibiting loss-of-heterozygosity or loss of expression at an allele encoding a non-target antigen; and/or of inhibiting the proliferation of an HLA-E positive cell exhibiting loss-of-heterozygosity at an allele encoding a non-target antigen. The methods may be performed in vitro or in vivo. As used herein, the term “contacting” refers to mixing cell populations in culture, administering a cellular therapy to a subject, or otherwise causing a target cell population (e.g. HLA-E positive cells) to come into contact with another cell population. Contacting can thus refer to both in vitro and in vivo methods.

Provided herein are methods of treating a subject in need thereof, comprising administering to the subject a therapeutically effective amount of a composition comprising immune cells comprising the engineered receptors of the disclosure.

In some embodiments, the method comprises treating an HLA-E positive cancer in a subject identified as having or suspected of having a loss of heterozygosity at an allele encoding a non-target antigen in the HLA-E positive cancer, comprising administering to the subject the immune cells described herein. In some embodiments, the immune cells are autologous. In some embodiments, the immune cells are allogeneic.

The disclosure provides methods of making an immune cell therapy. In some embodiments, the method comprises transforming immune cells with the polynucleotide system described herein. In some embodiments, the polynucleotide system comprises one or more polynucleotides comprising polynucleotide sequences encoding: an activator receptor comprising an extracellular antigen binding domain specific to an HLA-E antigen; and an inhibitory receptor comprising an extracellular antigen binding domain specific to a non-target antigen that is not expressed by the cancer cell due to a loss of heterozygosity in the cancer cell.

In some embodiments, the method of treating a subject comprises providing immune cells from a subject suffering from or at risk for HLA-E positive cancer. In some embodiments, the method comprises transducing the immune cell with the polynucleotide system described herein.

The current method for adoptive cell therapy using autologous cells includes isolating immune cells from patient blood, performing a series of modifications on the isolated cells, and administering the cells to a patient (Papathanasiou et al. Cancer Gene Therapy. 27:799-809 (2020)). Providing immune cells from a subject suffering from or at risk for cancer or a hematological malignancy requires isolation of immune cell from the patient's blood, and can be accomplished through methods known in the art, for example, by leukapheresis. During leukapheresis, blood from a subject is extracted and the peripheral blood mononuclear cells (PBMCs) are separated, and the remainder of the blood is returned to the subject's circulation. The PBMCs are stored either frozen or cryopreserved as a sample of immune cells and provided for further processing steps, such as, e.g. the modifications described herein.

In some embodiments, the method of treating a subject described herein comprises modifications to immune cells from the subject comprising a series of modifications comprising enrichment, activation, genetic modification, expansion, formulation, and cryopreservation.

The disclosure provides enrichment steps that can be, for example, washing and fractionating methods known in the art for preparation of subject PBMCs for downstream procedures, e.g. the modifications described herein. For example, without limitation, methods can include devices to remove gross red blood cells and platelet contaminants, systems for size-based cell fractionation for the depletion of monocytes and the isolation of lymphocytes, and/or systems that allow the enrichment of specific subsets of T cells, such as, e.g. CD4+, CD8+, CD25+, or CD62L+ T cells. Following the enrichment steps, a target sub-population of immune cells will be isolated from the subject PMBCs for further processing. Those skilled in the art will appreciate that enrichment steps, as provided herein, may also encompass any newly discovered method, device, reagent or combination thereof.

The disclosure provides activation steps that can be any method known in the art to induce activation of immune cells, e.g. T cells, required for their ex vivo expansion. Immune cell activation can be achieved, for example, by culturing the subject immune cells in the presence of dendritic cells, culturing the subject immune cells in the presence of artificial antigen-presenting cells (AAPCs), or culturing the immune cells in the presence of irradiated K562-derived AAPCs. Other methods for activating subject immune cells can be, for example, culturing the immune cells in the presence of isolated activating factors and compositions, e.g. beads, surfaces, or particles functionalized with activating factors. Activating factors can include, for example, antibodies, e.g. anti-CD3 and/or anti-CD28 antibodies. Activating factors can also be, for example, cytokines, e.g. interleukin (IL)-2 or IL-21. Activating factors can also be costimulatory molecules, such as, for example, CD40, CD40L, CD70, CD80, CD83, CD86, CD137L, ICOSL, GITRL, and CD134L. Those skilled in the art will appreciate that activating factors, as provided herein, may also encompass any newly discovered activating factor, reagent, composition, or combination thereof that can activate immune cells.

The disclosure provides genetic modification steps for modifying the subject immune cells. In some embodiments, the genetic modification comprises transducing the immune cell with an engineered receptor. In some embodiments, the method comprises transducing the immune cell with a first vector comprising a sequence encoding the activator receptor and a second vector comprising a sequence encoding the inhibitory receptor, thereby producing an immune cell expressing the activator and inhibitory receptors.

The disclosure provides expansion steps for the genetically modified subject immune cells. Genetically modified subject immune cells can be expanded in any immune cell expansion system known in the art to generate therapeutic doses of immune cells for administration. For example, bioreactor bags for use in a system comprising controller pumps, and probes that allow for automatic feeding and waste removal can be used for immune cell expansion. Cell culture flasks with gas-permeable membranes at the base may be used for immune cell expansion. Any such system known in the art that enables expansion of immune cells for clinical use is encompassed by the expansion step provided herein. Immune cells are expanded in culture systems in media formulated specifically for expansion. Expansion can also be facilitated by culturing the immune cell of the disclosure in the presence of activation factors as described herein. Those skilled in the art will appreciate that expansion steps, as provided herein, may also encompass any newly discovered culture systems, media, or activating factors that can be used to expand immune cells.

The disclosure provides formulation and cryopreservation steps for the expanded genetically modified subject immune cells. Formulation steps provided include, for example, washing away excess components used in the preparation and expansion of immune cells of the methods of treatment described herein. Any pharmaceutically acceptable formulation medium or wash buffer compatible with immune cell known in the art may be used to wash, dilute/concentration immune cells, and prepare doses for administration. Formulation medium can be acceptable for administration of the immune cells, such as, for example crystalloid solutions for intravenous infusion. Cryopreservation can optionally be used to store immune cells long-term. Cryopreservation can be achieved using known methods in the art, including for example, storing cells in a cryopreservation medium containing cryopreservation components. Cryopreservation components can include, for example, dimethyl sulfoxide or glycerol. Immune cells stored in cryopreservation medium can be cryopreserved by reducing the storage temperature to −80° C. to −180° C.

In some embodiments, the method comprises administering immune cells described herein. In some embodiments, the method comprises administering a conditioning regimen prior to administering the immune cells described herein. In some embodiments, the conditioning regimen is lymphodepletion. A lymphodepletion regimen can include, for example, administration of alemtuzumab, cyclophosphamide, benduamustin, rituximab, pentostatin, and/or fludarabine. Lymphodepletion regimen can be administered in one or more cycles until the desired outcome of reduced circulating immune cells.

In some embodiments, the conditioning regimen comprises administering an agent that specifically targets, and reduces or eliminates CD52+ cells in the subject, and the immune cells are modified to reduce or eliminate CD52 expression.

In some embodiments, the method of treatment comprises determining the HLA germline type of the subject. In some embodiments, determining the HLA germline type comprises determining the presence of HLA-A*01, HLA-A*02, HLA-A*03, HLA-A*11, HLA-B*07 or HLA-C*07 heterozygosity. In some embodiments, the HLA germline type is determined in bone marrow.

In some embodiments, the method of treatment comprises determining the level of expression of an activator antigen, e.g. an HLA-E antigen. In some embodiments, the level of expression of an activator antigen is determined in tumor tissue samples from the subject. In some embodiments, the expression level of an activator antigen is determined using next generation sequencing. In some embodiments, the expression level of an activator antigen is determined using RNA sequencing. In some embodiments, the level of an activator antigen is determined using immunohistochemistry.

In some embodiments, the method of treatment comprises determining if cells of the cancer do not express, or have lost expression of the inhibitor antigen. Suitable methods include taking a biopsy of the cancer followed by, inter alia, PCR amplification of inhibitor antigen sequences (RT-PCR, ddPCR and the like), sequencing genomic DNA of the cancer cells, and immunohistochemistry based methods to determine expression of the inhibitor antigen in cancer cells.

In some embodiments, the method of treatment comprises administering a therapeutically effective dose of immune cells in a subject in need thereof, wherein the subject is determined to be HLA germline HLA-A*01, HLA-A*02, HLA-A*03, HLA-A*11, HLA-B*07 or HLA-C*07 heterozygous and have tumor tissue with activator expression and loss of HLA-A*01, HLA-A*02, HLA-A*03, HLA-A*11, HLA-B*07 or HLA-C*07. In some embodiments, the method of treatment comprises administering a therapeutically effective dose of immune cells in a subject in need thereof, wherein the subject is determined to be HLA germline HLA-A*11 heterozygous and have tumor tissue with activator expression and loss of HLA-A*11.

In some embodiments, a therapeutically effective dose of the immune cells described herein are administered. In some embodiments, the immune cells of the disclosure are administered by intravenous injection. In some embodiments, the immune cells of the disclosure are administered by intraperitoneal injection. In some embodiments, a therapeutically effective dose comprises about 0.5×10⁶ cells, about 1×10⁶ cells, about 2×10⁶ cells, about 3×10⁶ cells, 4×10⁶ cells, about 5×10⁶ cells, about 6×10⁶ cells, about 7×10⁶ cells, about 8×10⁶ cells, about 9×10⁶ cells, about 1×10⁷, about 2×10⁷, about 3×10⁷, about 4×10⁷, about 5×10⁷, about 6×10⁷, about 7×10⁷, about 8×10⁷, about 9×10⁷, 1×10⁸ cells, about 2×10⁸ cells, about 3×10⁸ cells, about 4×10⁸ cells, about 5×10⁸ cells, or about 6×10⁸ cells. In some embodiments, a therapeutically effective dose comprises about 0.5×10⁶ cells to about 6×10⁸ cells, about 1×10⁶ cells to about 5×10⁸ cells, about 2×10⁶ cells to about 5×10⁸ cells, about 3×10⁶ cells to about 4×108 cells, about 4×10⁶ cells to about 3×10⁸ cells, about 5×10⁶ cells to about 2×108 cells, about 6×10⁶ cells to about 1×10⁸ cells, about 7×10⁶ cells to about 9×10⁷ cells, about 8×10⁶ cells to about 8×10⁷ cells, about 9×10⁶ cells to about 7×10⁷ cells, about 1×10⁷ cells to about 6×10⁷ cells, or about 2×10⁷ cells to about 5×10⁷ cells. In some embodiments, a therapeutically effective dose comprises about 0.5×10⁶ cells to about 6×10⁸ cells. The term “about” as referred to in a therapeutically dose, can be, for example, ±0.5×10⁶ cells, ±0.5×10⁷ cells, or ±0.5×10⁸ cells.

In some embodiments, the subject in need thereof has cancer. In some embodiments, the cancer is a HLA-E positive cancer. Cancer is a disease in which abnormal cells divide without control and spread to nearby tissue. In some embodiments, the cancer comprises a liquid tumor or a solid tumor. Exemplary liquid tumors include leukemias and lymphomas. Exemplary solid tumors include sarcomas and carcinomas. Cancers can arise in virtually an organ in the body, including blood, bone marrow, lung, breast, colon, rectal, bone, central nervous system, pancreas, prostate and ovary. Further cancers that are solid tumors include, for example, prostate cancer, testicular cancer, breast cancer, brain cancer, pancreatic cancer, colon cancer, thyroid cancer, stomach cancer, lung cancer, ovarian cancer, Kaposi's sarcoma, skin cancer, squamous cell skin cancer, renal cancer, head and neck cancers, throat cancer, squamous carcinomas that form on the moist mucosal linings of the nose, mouth, throat, bladder cancer, osteosarcoma, cervical cancer, endometrial cancer, esophageal cancer, liver cancer, and kidney cancer. In some embodiments, the condition treated by the methods described herein is metastasis of melanoma cells, prostate cancer cells, testicular cancer cells, breast cancer cells, brain cancer cells, pancreatic cancer cells, colon cancer cells, thyroid cancer cells, stomach cancer cells, lung cancer cells, ovarian cancer cells, Kaposi's sarcoma cells, skin cancer cells, renal cancer cells, head or neck cancer cells, throat cancer cells, squamous carcinoma cells, bladder cancer cells, osteosarcoma cells, cervical cancer cells, endometrial cancer cells, esophageal cancer cells, liver cancer cells, or kidney cancer cells.

Any cancer wherein a plurality of the cancer cells express the first, activator antigen and do not express the second, inhibitor antigen is envisaged as within the scope of the instant disclosure. For example, HLA-E positive cancers that do not express the inhibitor antigen that can be treated using the methods described herein include colorectal cancer, renal cancer, an ovarian cancer, cervical cancer, melanoma, urothelial cancer, pancreatic cancer, gastric cancer, head and neck cancer, lung cancer, breast cancer and lymphoma.

Administration of the immune cells or pharmaceutical compositions described herein can reduce the size of a tumor in the subject. In some embodiments, the size of the tumor is reduced by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, relative to the size of the tumor before administration of the immune cells or pharmaceutical compositions. In some embodiments, the tumor is eliminated.

Administration of the immune cells or pharmaceutical compositions described herein can arrest the growth of a tumor in the subject. For example, the immune cells or pharmaceutical compositions can kill tumor cells, so that the tumor stops growing, or is reduced in size. In some cases, immune cells or pharmaceutical compositions can prevent formation of additional tumors, or reduce the total number of tumors in the subject.

Administration of the immune cells or pharmaceutical compositions described herein can result in selective killing of a cancer cell but not a wild-type cell in the subject. In some embodiments, about 60% of the cells killed are cancer cells, about 65% of the cells killed are cancer cells, about 70% of the cells killed are cancer cells, about 75% of the cells killed are cancer cells, about 80% of the cells killed are cancer cells, about 85% of the cells killed are cancer cells, about 90% of the cells killed are cancer cells, about 95% of the cells killed are cancer cells, or about 100% of the cells killed are cancer cells.

Administration of the immune cells or pharmaceutical compositions described herein can result in the killing of about 40%, about 50%, about 60%, about 70%, about 80%, about 90% or all of the cancer cells of the subject.

Administration of the immune cells or pharmaceutical compositions described herein can result in fewer side effects for the subject than administration of an otherwise equivalent immune cell comprising the first activator receptor but no second inhibitory receptor. For example, administering the immune cells or pharmaceutical compositions described herein can reduce dose limited toxicity relative to the HLA-E CAR, or HLA-E TCR administered without the second inhibitory receptor.

Methods of genotyping cancer cells and normal cells from a subject for the presence or absence of SNPs will be readily apparent to persons of ordinary skill in the art. SNP genotyping methods include, inter alia, PCR based methods such as dual-probe TaqMan assays, array-based hybridization methods and sequencing.

Methods of measuring the expression of the target antigen in cancer or wild-type cells from a subject will be readily apparent to persons of ordinary skill in the art. These include, inter alia, methods of measuring RNA expression such as RNA sequencing and reverse transcription polymerase chain reaction (RT-PCR), as well as methods of measuring protein expression such as immunohistochemistry based methods. Methods of measuring loss of heterozygosity in a plurality of cancer cells, include, inter alia, high throughput sequencing of genomic DNA extracted from cancer cells using methods known in the art.

In some embodiments, the immune cells are T cells.

In some embodiments, the immune cells are allogeneic or autologous.

In some embodiments, the second receptor increases the specificity of the immune cells for the HLA-E-positive cancer cells compared to immune cells that express the first receptor but do not express the second receptor. In some embodiments, the immune cells have reduced side effects compared to immune cells that express the first receptor but do not express the second receptor.

Treating cancer can result in a reduction in size of a tumor. A reduction in size of a tumor may also be referred to as “tumor regression”. Preferably, after treatment, tumor size is reduced by 5% or greater relative to its size prior to treatment; more preferably, tumor size is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Size of a tumor may be measured by any reproducible means of measurement. The size of a tumor may be measured as a diameter of the tumor.

Treating cancer can result in a reduction in tumor volume. Preferably, after treatment, tumor volume is reduced by 5% or greater relative to its size prior to treatment; more preferably, tumor volume is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Tumor volume may be measured by any reproducible means of measurement.

Treating cancer results in a decrease in number of tumors. Preferably, after treatment, tumor number is reduced by 5% or greater relative to number prior to treatment; more preferably, tumor number is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. Number of tumors may be measured by any reproducible means of measurement. The number of tumors may be measured by counting tumors visible to the naked eye or at a specified magnification. Preferably, the specified magnification is 2×, 3×, 4×, 5×, 10×, or 50×.

Treating cancer can result in a decrease in number of metastatic lesions in other tissues or organs distant from the primary tumor site. Preferably, after treatment, the number of metastatic lesions is reduced by 5% or greater relative to number prior to treatment; more preferably, the number of metastatic lesions is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. The number of metastatic lesions may be measured by any reproducible means of measurement. The number of metastatic lesions may be measured by counting metastatic lesions visible to the naked eye or at a specified magnification. Preferably, the specified magnification is 2×, 3×, 4×, 5×, 10×, or 50×.

Treating cancer can result in an increase in average survival time of a population of treated subjects in comparison to a population receiving carrier alone. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in an increase in average survival time of a population of treated subjects in comparison to a population of untreated subjects. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in increase in average survival time of a population of treated subjects in comparison to a population receiving monotherapy with a drug that is not a compound of the present invention, or a pharmaceutically acceptable salt, prodrug, metabolite, analog or derivative thereof. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in a decrease in the mortality rate of a population of treated subjects in comparison to a population receiving carrier alone. Treating cancer can result in a decrease in the mortality rate of a population of treated subjects in comparison to an untreated population. Treating cancer can result in a decrease in the mortality rate of a population of treated subjects in comparison to a population receiving monotherapy with a drug that is not a compound of the present invention, or a pharmaceutically acceptable salt, prodrug, metabolite, analog or derivative thereof. Preferably, the mortality rate is decreased by more than 2%; more preferably, by more than 5%; more preferably, by more than 10%; and most preferably, by more than 25%. A decrease in the mortality rate of a population of treated subjects may be measured by any reproducible means. A decrease in the mortality rate of a population may be measured, for example, by calculating for a population the average number of disease-related deaths per unit time following initiation of treatment with an active compound. A decrease in the mortality rate of a population may also be measured, for example, by calculating for a population the average number of disease-related deaths per unit time following completion of a first round of treatment with an active compound.

Treating cancer can result in a decrease in tumor growth rate. Preferably, after treatment, tumor growth rate is reduced by at least 5% relative to number prior to treatment; more preferably, tumor growth rate is reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Tumor growth rate may be measured by any reproducible means of measurement. Tumor growth rate can be measured according to a change in tumor diameter per unit time.

Treating cancer can result in a decrease in tumor regrowth. Preferably, after treatment, tumor regrowth is less than 5%; more preferably, tumor regrowth is less than 10%; more preferably, less than 20%; more preferably, less than 30%; more preferably, less than 40%; more preferably, less than 50%; even more preferably, less than 50%; and most preferably, less than 75%. Tumor regrowth may be measured by any reproducible means of measurement. Tumor regrowth is measured, for example, by measuring an increase in the diameter of a tumor after a prior tumor shrinkage that followed treatment. A decrease in tumor regrowth is indicated by failure of tumors to reoccur after treatment has stopped.

Treating or preventing a cell proliferative disorder can result in a reduction in the rate of cellular proliferation. Preferably, after treatment, the rate of cellular proliferation is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The rate of cellular proliferation may be measured by any reproducible means of measurement. The rate of cellular proliferation is measured, for example, by measuring the number of dividing cells in a tissue sample per unit time.

Treating or preventing a cell proliferative disorder can result in a reduction in the proportion of proliferating cells. Preferably, after treatment, the proportion of proliferating cells is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The proportion of proliferating cells may be measured by any reproducible means of measurement. Preferably, the proportion of proliferating cells is measured, for example, by quantifying the number of dividing cells relative to the number of nondividing cells in a tissue sample. The proportion of proliferating cells can be equivalent to the mitotic index.

Treating or preventing a cell proliferative disorder can result in a decrease in size of an area or zone of cellular proliferation. Preferably, after treatment, size of an area or zone of cellular proliferation is reduced by at least 5% relative to its size prior to treatment; more preferably, reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Size of an area or zone of cellular proliferation may be measured by any reproducible means of measurement. The size of an area or zone of cellular proliferation may be measured as a diameter or width of an area or zone of cellular proliferation.

Treating or preventing a cell proliferative disorder can result in a decrease in the number or proportion of cells having an abnormal appearance or morphology. Preferably, after treatment, the number of cells having an abnormal morphology is reduced by at least 5% relative to its size prior to treatment; more preferably, reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. An abnormal cellular appearance or morphology may be measured by any reproducible means of measurement. An abnormal cellular morphology can be measured by microscopy, e.g., using an inverted tissue culture microscope. An abnormal cellular morphology can take the form of nuclear pleiomorphism.

### Dosage and Administration

The immune cells and of the present disclosure may be administered in a number of ways depending upon whether local or systemic treatment is desired.

In some embodiments, administration is parenteral.

Methods for administration of cells for adoptive cell therapy are known and may be used in connection with the provided methods and compositions. For example, adoptive T cell therapy methods are described, e.g., in US Patent Application Publication No. 2003/0170238 to Gruenberg et al and U.S. Pat. No. 4,690,915 to Rosenberg.

The compositions of the disclosure are suitable for parenteral administration. As used herein, “parenteral administration” of a pharmaceutical composition includes any route of administration characterized by physical breaching of a tissue of a subject and administration of the pharmaceutical composition through the breach in the tissue, thus generally resulting in the direct administration into the blood stream, into muscle, or into an internal organ. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal, intravenous, intraarterial, intrathecal, intraventricular, intraurethral, intracranial, intratumoral, intrasynovial injection or infusions; and kidney dialytic infusion techniques. In some embodiments, parenteral administration of the compositions of the present disclosure comprises intravenous or intraarterial administration.

The disclosure provides pharmaceutical compositions comprising a plurality of immune cells of the disclosure, and a pharmaceutically acceptable carrier, diluent or excipient.

Formulations of a pharmaceutical composition suitable for parenteral administration typically generally comprise of immune cells combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampoules or in multi-dose containers containing a preservative. Formulations for parenteral administration include, but are not limited to, suspensions, solutions, emulsions in oily or aqueous vehicles, pastes, and the like. Such formulations may further comprise one or more additional ingredients including, but not limited to, suspending, stabilizing, or dispersing agents. Parenteral formulations also include aqueous solutions which may contain excipients such as salts, carbohydrates and buffering agents. Exemplary parenteral administration forms include solutions or suspensions in sterile aqueous solutions, for example, aqueous propylene glycol or dextrose solutions. Such dosage forms can be suitably buffered, if desired. Formulations for parenteral administration may be formulated to be immediate and/or modified release. Modified release formulations include delayed-, sustained-, pulsed-, controlled-, targeted and programmed release.

In some embodiments, the formulated composition comprising the immune cells is suitable for administration via injection. In some embodiments, the formulated composition comprising the immune cells is suitable for administration via infusion.

The pharmaceutical compositions of the present disclosure, which may conveniently be presented in unit dosage form, may be prepared according to conventional techniques well known in the pharmaceutical industry. Such techniques include the step of bringing into association the immune cells with the pharmaceutical carrier(s) or excipient(s), such as liquid carriers.

Aqueous suspensions may further contain substances that increase the viscosity of the suspension including, for example, sodium carboxymethylcellulose, sorbitol and/or dextran. The suspension may also contain stabilizers.

The compositions of the present disclosure may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present disclosure, such as dyes, preservatives, antioxidants, opacifiers, thickening agents and stabilizers. However, such materials, when added, should not unduly interfere with the biological activities of the immune cells of the compositions of the present disclosure.

The formulation or composition may also contain more than one active ingredient useful for the particular indication, disease, or condition being treated with the immune cells, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents.

The pharmaceutical composition in some aspects can employ time-released, delayed release, and sustained release delivery systems such that the delivery of the composition occurs prior to, and with sufficient time to cause, sensitization of the site to be treated. Many types of release delivery systems are available and known. Such systems can avoid repeated administrations of the composition, thereby increasing convenience to the subject and the physician.

Administration can be effected in one dose, continuously or intermittently throughout the course of treatment. Single or multiple administrations can be carried out with the dose level and pattern being selected by the treating physician.

The pharmaceutical composition in some embodiments contains the immune cells in amounts effective to treat or prevent a cancer, such as a therapeutically effective or prophylactically effective amount. Therapeutic or prophylactic efficacy in some embodiments is monitored by periodic assessment of treated subjects. For repeated administrations over days, weeks or months, depending on the condition, the treatment can be repeated until a desired suppression of cancer signs or symptoms occurs. However, other dosage regimens may be useful and can be determined. The desired dosage can be delivered by a single bolus administration or infusion of the composition or by multiple bolus administrations or infusions of the composition.

The cells or population of cells can be administrated in one or more doses. In some embodiments, an effective amount of cells can be administrated as a single dose. In some embodiments, an effective amount of cells can be administrated as more than one doses over a period time. Timing of administration is within the judgment of a managing physician and depends on the clinical condition of the patient.

The cells or population of cells may be obtained from any source, such as a blood bank or a donor, or the patient themselves.

An effective amount means an amount which provides a therapeutic or prophylactic benefit. The dosage administered will be dependent upon the age, health and weight of the recipient, kind of concurrent treatment, if any, frequency of treatment and the nature of the effect desired. In some embodiments, an effective amount of cells or composition comprising those cells are administrated parenterally. In some embodiments, administration can be an intravenous administration. In some embodiments, administration can be directly done by injection within a tumor.

For purposes of the disclosure, an assay, which comprises, for example, comparing the extent to which target cells are lysed or one or more cytokines are secreted by immune cells expressing the receptors, upon administration of a given dose of such immune cells to a mammal, among a set of mammals of which is each given a different dose of the immune cells, can be used to determine a starting dose to be administered to a mammal.

In some embodiments, the cells are administered as part of a combination treatment, such as simultaneously with or sequentially with, in any order, another therapeutic intervention, such as an antibody or engineered cell or receptor or agent, such as a cytotoxic or therapeutic agent. The immune cells of the disclosure are in some embodiments are co-administered with one or more additional therapeutic agents or in connection with another therapeutic intervention, either simultaneously or sequentially in any order. In some contexts, the immune cells are co-administered with another therapy sufficiently close in time such that the immune cell populations enhance the effect of one or more additional therapeutic agents, or vice versa. In some embodiments, the immune cells are administered prior to the one or more additional therapeutic agents. In some embodiments, the immune cells are administered after to the one or more additional therapeutic agents.

In embodiments, a lymphodepleting chemotherapy is administered to the subject prior to, concurrently with, or after administration (e.g., infusion) of adoptive immune cells. In an example, the lymphodepleting chemotherapy is administered to the subject prior to administration of the immune cells. For example, the lymphodepleting chemotherapy ends 1-4 days (e.g., 1, 2, 3, or 4 days) prior to adoptive cell infusion. In embodiments, multiple doses of adoptive cells are administered, e.g., as described herein. In embodiments, a lymphodepleting chemotherapy is administered to the subject prior to, concurrently with, or after administration (e.g., infusion) of the immune cells described herein. Examples of lymphodepletion include, but may not be limited to, nonmyeloablative lymphodepleting chemotherapy, myeloablative lymphodepleting chemotherapy, total body irradiation, etc. Examples of lymphodepleting agents include, but are not limited to, antithymocyte globulin, anti-CD3 antibodies, anti-CD4 antibodies, anti-CD8 antibodies, anti-CD52 antibodies, anti-CD2 antibodies, TCRαβ blockers, anti-CD20 antibodies, anti-CD19 antibodies, Bortezomib, rituximab, anti-CD 154 antibodies, rapamycin, CD3 immunotoxin, fludarabine, cyclophosphamide, busulfan, melphalan, Mabthera, Tacrolimus, alefacept, alemtuzumab, OKT3, OKT4, OKT8, OKT11, fingolimod, anti-CD40 antibodies, anti-BR3 antibodies, Campath-111, anti-CD25 antibodies, calcineurin inhibitors, mycophenolate, and steroids, which may be used alone or in combination. As a further example, a lymphodepletion regimen can include, administration of alemtuzumab, cyclophosphamide, benduamustin, rituximab, pentostatin, and/or fludarabine. Lymphodepletion regimen can be administered in one or more cycles until the desired outcome of reduced circulating immune cells. In some embodiments, the lymphodepletion comprises administering an agent that specifically targets, and reduces or eliminates CD52+ cells in the subject, and the immune cells are modified to reduce or eliminate CD52 expression.

In some embodiments, an immune stimulating therapy is administered to the subject prior to, concurrently with, or after administration (e.g. infusion) of adoptive immune cells. In some embodiments, the immune stimulating therapy comprises homeostatic cytokines. In some embodiments, the immune stimulating therapy comprises immune-stimulatory molecules. In some embodiments, the immune stimulating therapy comprises IL-2, IL-7, IL-12, IL-15, IL-21, IL-9, or a functional fragment thereof. In some embodiments, the immune stimulating therapy comprises IL-2, IL-7, IL-12, IL-15, IL-21, IL-9, or combinations thereof. In some embodiments, the immune stimulating therapy comprises IL-2, or a functional fragment thereof.

Methods for adoptive cell therapy using autologous cells includes isolating immune cells from patient blood, performing a series of modifications on the isolated cells including transducing the cells with one or more vectors encoding the dual receptor system described herein, and administering the cells to a patient. Providing immune cells from a subject suffering from or at risk for cancer or a hematological malignancy requires isolation of immune cell from the patient's blood, and can be accomplished through methods known in the art, for example, by leukapheresis. During leukapheresis, blood from a subject is extracted and the peripheral blood mononuclear cells (PBMCs) are separated, and the remainder of the blood is returned to the subject's circulation. The PBMCs are stored either frozen or cryopreserved as a sample of immune cells and provided for further processing steps, such as, e.g. the modifications described herein.

In some embodiments, the method of treating a subject described herein comprises modifications to immune cells from the subject comprising a series of modifications comprising enrichment and/or depletion, activation, genetic modification, expansion, formulation, and cryopreservation.

The disclosure provides enrichment and/or depletion steps that can be, for example, washing and fractionating methods known in the art for preparation of subject PBMCs for downstream procedures, e.g. the modifications described herein. For example, without limitation, methods can include devices to remove gross red blood cells and platelet contaminants, systems for size-based cell fractionation for the depletion of monocytes and the isolation of lymphocytes, and/or systems that allow the enrichment of specific subsets of T cells, such as, e.g. CD4+, CD8+, CD25+, or CD62L+ T cells. Following the enrichment steps, a target sub-population of immune cells will be isolated from the subject PMBCs for further processing. Those skilled in the art will appreciate that enrichment steps, as provided herein, may also encompass any newly discovered method, device, reagent or combination thereof.

The disclosure provides activation steps that can be any method known in the art to induce activation of immune cells, e.g. T cells, required for their ex vivo expansion. Immune cell activation can be achieved, for example, by culturing the subject immune cells in the presence of dendritic cells, culturing the subject immune cells in the presence of artificial antigen-presenting cells (AAPCs), or culturing the immune cells in the presence of irradiated K562-derived AAPCs. Other methods for activating subject immune cells can be, for example, culturing the immune cells in the presence of isolated activating factors and compositions, e.g. beads, surfaces, or particles functionalized with activating factors. Activating factors can include, for example, antibodies, e.g. anti-CD3 and/or anti-CD28 antibodies. Activating factors can also be, for example, cytokines, e.g. interleukin (IL)-2 or IL-21. Activating factors can also be costimulatory molecules, such as, for example, CD40, CD40L, CD70, CD80, CD83, CD86, CD137L, ICOSL, GITRL, and CD134L. Those skilled in the art will appreciate that activating factors, as provided herein, may also encompass any newly discovered activating factor, reagent, composition, or combination thereof that can activate immune cells.

The disclosure provides genetic modification steps for modifying the subject immune cells. In some embodiments, the genetic modification comprises transducing the immune cell with a vector comprising a shRNA described herein complementary to B2M or HLA-A. In some embodiments, the genetic modification comprises modifying the genome of the immune cells to induce mutations in B2M or HLA-A using CRISPR/Cas mediated genome engineering. In some embodiments, the method comprises transducing the immune cell with one or more vectors encoding the activator and inhibitory receptors, thereby producing immune cells expressing the activator and inhibitory receptors.

The disclosure provides expansion steps for the genetically modified subject immune cells. Genetically modified subject immune cells can be expanded in any immune cell expansion system known in the art to generate therapeutic doses of immune cells for administration. For example, bioreactor bags for use in a system comprising controller pumps, and probes that allow for automatic feeding and waste removal can be used for immune cell expansion. Cell culture flasks with gas-permeable membranes at the base may be used for immune cell expansion. Any such system known in the art that enables expansion of immune cells for clinical use is encompassed by the expansion step provided herein. Immune cells are expanded in culture systems in media formulated specifically for expansion. Expansion can also be facilitated by culturing the immune cell of the disclosure in the presence of activation factors as described herein. Those skilled in the art will appreciate that expansion steps, as provided herein, may also encompass any newly discovered culture systems, media, or activating factors that can be used to expand immune cells.

The disclosure provides formulation and cryopreservation steps for the expanded genetically modified subject immune cells. Formulation steps provided include, for example, washing away excess components used in the preparation and expansion of immune cells of the methods of treatment described herein. Any pharmaceutically acceptable formulation medium or wash buffer compatible with immune cell known in the art may be used to wash, dilute/concentration immune cells, and prepare doses for administration. Formulation medium can be acceptable for administration of the immune cells, such as, for example crystalloid solutions for intravenous infusion.

Cryopreservation can optionally be used to store immune cells long-term. Cryopreservation can be achieved using known methods in the art, including for example, storing cells in a cryopreservation medium containing cryopreservation components. Cryopreservation components can include, for example, dimethyl sulfoxide or glycerol. Immune cells stored in cryopreservation medium can be cryopreserved by reducing the storage temperature to −80° C. to −196° C.

In some embodiments, the method of treatment comprises determining the HLA germline type of the subject. In some embodiments, the HLA germline type is determined in bone marrow.

In some embodiments, the method of treatment comprises determining the level of expression of HLA-E. In some embodiments, the level of expression of HLA-E is determined in tumor tissue samples from the subject. In some embodiments, the expression level of HLA-E is determined using next generation sequencing. In some embodiments, the expression level of HLA-E is determined using RNA sequencing. In some embodiments, the level of HLA-E is determined using immunohistochemistry.

In some embodiments, the method of treatment comprises administering a therapeutically effective dose of immune cells comprising an HLA-A*02 inhibitory receptor to a subject in need thereof, wherein the subject is determined to be HLA germline HLA-A*02 heterozygous and have cancer cells with loss of HLA-A*02. In some embodiments, the method of treatment comprises administering a therapeutically effective dose of immune cells comprising an HLA-A*01, HLA-A*03, HLA-A*07, HLA-C*07, or HLA-B*07 inhibitory receptor to a subject in need thereof, wherein the subject is determined to be HLA germline HLA-A*01, HLA-A*03, HLA-A*07, HLA-C*07, or HLA-B*07 heterozygous and have cancer cells with loss of HLA-A*01, HLA-A*03, HLA-A*07, HLA-C*07, or HLA-B*07, respectively.

In various embodiments, the disclosure provides method of treatment of heterozygous HLA-A*02 patients with malignancies that express HLA-E and have lost HLA-A*02 expression; and/or of treatment of heterozygous HLA-A*02 adult patients with recurrent unresectable or metastatic solid tumors that express HLA-E and have lost HLA-A*02 expression.

In various embodiments, the disclosure provides method of treatment of heterozygous HLA-A*03 patients with malignancies that express HLA-E and have lost HLA-A*03 expression; and/or of treatment of heterozygous HLA-A*03 adult patients with recurrent unresectable or metastatic solid tumors that express HLA-E and have lost HLA-A*03 expression.

In some embodiments, a therapeutically effective dose of the immune cells described herein are administered. In some embodiments, the immune cells of the disclosure are administered by intravenous injection. In some embodiments, the immune cells of the disclosure are administered by intraperitoneal injection. In some embodiments, a therapeutically effective dose comprises about 0.5×10⁶ cells, about 1×10⁶ cells, about 2×10⁶ cells, about 3×10⁶ cells, 4×10⁶ cells, about 5×10⁶ cells, about 6×10⁶ cells, about 7×10⁶ cells, about 8×10⁶ cells, about 9×10⁶ cells, about 1×10⁷, about 2×10⁷, about 3×10⁷, about 4×10⁷, about 5×10⁷, about 6×10⁷, about 7×10⁷, about 8×10⁷, about 9×10⁷, about 1×10⁸ cells, about 2×10⁸ cells, about 3×10⁸ cells, about 4×10⁸ cells, about 5×10⁸ cells, about 6×10⁸ cells, about 7×10⁸ cells, about 8×10⁸ cells, about 9×10⁸ cells, about 1×10⁹ cells, about 2×10⁹ cells, about 3×10⁹ cells, about 3×10⁹ cells, about 4×10⁹ cells, about 5×10⁹ cells, about 5×10⁹ cells, about 6×10⁹ cells, about 7×10⁹ cells, about 8×10⁹ cells, about 9×10⁹ cells, about 1×10¹⁰ cells, about 2×10¹⁰ cells, about 3×10¹⁰ cells, about 4×10¹⁰ cells, about 5×10¹⁰ cells, about 6×10¹⁰ cells, about 7×10¹⁰ cells, about 8×10¹⁰ cells, or about 9×10¹⁰ cells.

In some embodiments, a therapeutically effective dose comprises about 0.5×10⁶ cells to about 9×10¹⁰ cells, about 1×10⁶ cells to about 5×10¹⁰ cells, about 2×10⁶ cells to about 5×10⁹ cells, about 3×10⁶ cells to about 5×10⁹ cells, about 4×10⁶ cells to about 3×10⁹ cells, about 5×10⁶ cells to about 2×10⁹ cells, about 6×10⁶ cells to about 1×10⁹ cells, 0.5×10⁶ cells to about 6×10⁹ cells, about 1×10⁶ cells to about 5×10⁹ cells, about 2×10⁶ cells to about 5×10⁹ cells, about 3×10⁶ cells to about 4×10⁹ cells, about 4×10⁶ cells to about 3×10⁹ cells, about 5×10⁶ cells to about 2×10⁹ cells, about 6×10⁶ cells to about 1×10⁹ cells, 0.5×10⁶ cells to about 6×10⁸ cells, about 1×10⁶ cells to about 5×10⁸ cells, about 2×10⁶ cells to about 5×10⁸ cells, about 3×10⁶ cells to about 4×10⁸ cells, about 4×10⁶ cells to about 3×10⁸ cells, about 5×10⁶ cells to about 2×10⁸ cells, about 6×10⁶ cells to about 1×10⁸ cells, about 7×10⁶ cells to about 9×10⁸ cells, about 8×10⁶ cells to about 8×10⁸ cells, about 9×10⁶ cells to about 7×10⁸ cells, about 1×10⁷ cells to about 6×10⁸ cells, about 2×10⁷ cells to about 5×10⁸ cells, about 7×10⁶ cells to about 9×10⁷ cells, about 8×10⁶ cells to about 8×10⁷ cells, about 9×10⁶ cells to about 7×10⁷ cells, about 1×10⁷ cells to about 6×10⁷ cells, or about 2×10⁷ cells to about 5×10⁷ cells.

In some embodiments, a therapeutically effective dose comprises about 0.5×10⁵ cells to about 9×10¹⁰ cells. In some embodiments, a therapeutically effective dose comprises about 0.5×10⁶ cells to about 1×10¹⁰ cells. In some embodiments, a therapeutically effective dose comprises about 0.5×10⁶ cells to about 5×10⁹ cells. In some embodiments, a therapeutically effective dose comprises about 0.5×10⁶ cells to about 1×10⁹ cells. In some embodiments, a therapeutically effective dose comprises about 0.5×10⁶ cells to about 6×10⁸ cells. In some embodiments, a therapeutically effective dose comprises about 0.5×10⁶ cells to about 9×10¹⁰ cells. In some embodiments, a therapeutically effective dose comprises about 0.5×10⁷ cells to about 1×10¹⁰ cells. In some embodiments, a therapeutically effective dose comprises about 0.5×10⁷ cells to about 5×10⁹ cells. In some embodiments, a therapeutically effective dose comprises about 0.5×10⁷ cells to about 1×10⁹ cells. In some embodiments, a therapeutically effective dose comprises about 0.5×10⁷ cells to about 6×10⁸ cells. In some embodiments, a therapeutically effective dose comprises about 0.5×10⁸ cells to about 9×10¹⁰ cells. In some embodiments, a therapeutically effective dose comprises about 0.5×10⁸ cells to about 1×10¹⁰ cells. In some embodiments, a therapeutically effective dose comprises about 0.5×10⁸ cells to about 5×10⁹ cells. In some embodiments, a therapeutically effective dose comprises about 0.5×10⁸ cells to about 1×10⁹ cells. The term “about” as referred to in a therapeutically dose, can be, for example, ±0.5×10⁶ cells, 0.5×10⁷ cells, or 0.5×10⁸ cells.

### Kits and Articles of Manufacture

The disclosure provides kits and articles of manufacture comprising the polynucleotides and vectors encoding the engineered receptors described herein, and immune cells edited using gene editing systems described herein and comprising the engineered receptors described herein. In some embodiments, the kit comprises articles such as vials, syringes and instructions for use.

In some embodiments, the kit comprises a polynucleotide or vector comprising a sequence encoding one or more engineered receptors of the disclosure.

In some embodiments, the kit comprises a plurality of immune cells comprising an engineered receptor as described herein. In some embodiments, the plurality of immune cells comprises a plurality of T cells.

### EXAMPLES

### Example 1: Methods

### Cell Culture

Jurkat cells encoding an NFAT luciferase reporter were obtained from BPS Bioscience. In culture, Jurkat cells were maintained in RPMI media supplemented with 10% FBS, 1% Pen/Strep and 0.4 mg/mL G418/Geneticin. HeLa cells were maintained as suggested by ATCC. B2M was knocked out (KO) using CRISPR/Cas9 to avoid self-activation, using a gRNA with a targeting sequence of AGGGTAGGAGAGACTCACGC (SEQ ID NO: 621) and standard methods.

### Jurkat Cell Transfection

Jurkat cells (NFAT-luc; B2M KO) were transiently transfected via 100 μL or 20 μl format 4D Nucleofactor (Lonza) according to manufacturer's protocol. Cotransfection was performed with 1-3 μg of activator receptor construct and 1-3 μg of inhibitory receptor construct per 1e6 cells and recovered in RPMI media supplemented with 20% heat-inactivated FBS and 0.1% Pen/Strep.

### Jurkat-NFAT-Luciferase Activation Studies

Briefly, Jurkat NFAT-Firefly-Luciferase cells were transfected with HLA-E activator (HLA-E scFv comprising a light chain variable region of SEQ ID NO: 7 and a heavy chain variable region of SEQ ID NO: 8, a CD8 hinge, CD28 transmembrane domain, and CD28/41BB/CD3 intracellular domain, third generation CAR) and an HLA-A*02 inhibitory (blocker) receptor (HLA-A*02 scFv of SEQ ID NO: 63, and LILRB1 hinge, transmembrane and intracellular domains, see SEQ ID NO: 264) constructs using Lonza 4D Nucleofector™ (AAF-1002B) or Neon™ transfection systems (ThermoFisher, MPK5000). Target HeLa cells, or target HeLa cells transfected (10,000 cells/well) with the indicated amount of HLA-A*02 mRNA synthesized as described below were added to transfected Jurkat-NFAT-Firefly-Luciferase cells (12,000 cells/well) to a final volume of 20 mL in 384-well plates (Corning 3570). After a 6-hour incubation at 37° C., the One-Step™ Luciferase firefly assay system (BPS Bioscience, 60690) was used to determine luminescence intensity on a Tecan Infinite® M1000.

### Primary T Cell Transduction and Culture

Primary T cells were stimulated with TransAct and transduced with the HLA-E CAR lentivirus, or with lentiviruses encoding the HLA-E CAR and an HLA-A*02 inhibitory receptor on the following day. After 24-hour transduction, CRISPR/Cas9 was used to knock out the B2M gene in the primary T cells as described above. Untransduced primary T cells in which the B2M gene had been knocked out by CRISPR/Cas9 were used as the control in the cytotoxicity assay. The populations of cells that were B2M- and positive for either the HLA-E CAR, or the HLA-E CAR and the HLA-A*02 inhibitory receptor, were confirmed by flow cytometry. Cells were purified to enrich for HLA-E activator and HLA-A2 inhibitory receptor expression. Purified cells were cultured in X-VIVO 15 medium containing 1% human serum and 300 U/ml recombinant human IL-2.

### Cytotoxicity Assay in Primary T Cells

HeLa cells expressing rLuc-RFP were transfected with HLA-E*0103 or HLA-A*02 mRNA and cultured in Lymphol medium containing 1% human serum. To investigate the sensitivity of HLA-E CAR activator and HLA-A*02 LIR1 blocker, HLA-E KO and wild-type HeLa cells (which are HLA-A*02 negative) were used, respectively. On day following transfection, 4000 primary T cells were co-cultured with 4000 transfected HeLa cells, which were plated in 384-well plates. Images of surviving HeLa cells were taken every 2 hours for at least 48 hours by Incucyte. Cytotoxicity of primary T cells was measured by specific killing (%).

In Vitro Transcription of mRNA

HLA-A*02 or HLA-E sequences were synthesized with a T7 promoter in the 5′ end together with 5′ and 3′ synthetic UTR sequences. In vitro transcription was done in 1×IVT buffer (40 mM Tris, 10 mM DTT, 2 mM Spermidine, 0.002% Triton X-100, and 27 mM MgCl₂) with T7 RNA polymerase (NEB, M0251S), Inorganic pyrophosphatase (NEB, M2403S) and murine RNase inhibitor (NEB, M0314S). In addition, 500 ng PCR purified template, 5 mM CleanCap Cap1 AG trimer, 5 mM each of ATP, CTP, GTP and pseudo-uridine triphosphate (pseudo-UTP) were added, and the transcription reaction was incubated at 37° C. for 2 hours, followed by addition of DNase I (NEB, M0303S) for additional 15 minutes incubation at 37° C.

The In vitro transcription reaction was finalized by the addition of polyA tailing with polyA enzyme (NEB, M0276S), and incubation at 37° C. for 30 minutes. The mRNA was then cleaned using the NEB Monarch kit. The purified mRNA was treated with Antarctic phosphatase (NEB, M0289S) for 1 hour at 37° C. in 1× Antarctic phosphatase buffer (NEB). mRNA was then purified again using the NEB Monarch kit. mRNA concentrations were measured by Nanodrop. The quality of mRNA was accessed by gel electrophoresis. The final in vitro transcribed mRNA was aliquoted and stored at −80° C. until shortly before use.

### Beta-2Microglobulin (B2M) Knockout

Jurkat cells were transfected with pre-complexed Cas9:sgRNA complexes, with an sgRNA specific to B2M. Jurkat cells without gRNA transfection and B2M stable knockout cells served as positive and negative controls, respectively. Four days after transfection, cells were stained for either HLA-A*02 expression or expression of Class I HLA using fluorescently labeled antibodies and subject to FACS analysis to confirm B2M knockout. The B2M gRNA had a targeting sequence of sequence AGGGTAGGAGAGACTCACGC (SEQ ID NO: 621).

### HLA-E Knockout in HeLa Cells

HeLa cells were transfected with pre-complexed Cas9:sgRNA complexes, with an sgRNA specific to HLA-E. Four days after transfection, cells were stained for HLA-E expression using fluorescently labeled antibody and subject to FACS analysis to confirm HLA-E knockout. HeLa cells with or without HLA-E stable knockout served as parental cells for HLA-E and HLA-A mRNA titration to determine EC50 and IC50, respectively. The GLA gRNA had a targeting sequence of GAGATAATCCTTGCCGTCGT (SEQ TD NO: 21944).

Sequences of the HLA-E activator are provided in Table 11 below.

TABLE 11
HLA-E Activator sequences

| Feature | Sequence |
|---|---|
| Full length | MEFGLSWLFLVAILKGVQCQVQLRESGPSLVKPSQTLSLTCTVS |
| activator | GFSLTSYGVHWVRQPPGKGLEWLGVIWTGGTTNYNTALMSRL |
| receptor | SITKDNSKSQVFLKMNSLQTDDTAIYYCARDGDSYNREAWFAY |
| | WGQGTLVTVSAGGGGSGGGGSGGGGSGGQIVLTQSPAVISASP |
| | GEKVILTCSASSSVSYMHWFQQKPGTSPKLWIYGTSNLASGVP |
| | ARFSGGGSGTSYSLTISRMEAEDAATYYCQQRSRYPFTFGSGTK |
| | LEIKTTTPAPRPPTPAPTIASQPLSLRPEACRPAAGGAVHTRGLD |
| | FACDFWVLVVVGGVLACYSLLVTVAFIIFWVRSKRSRLLHSDY |
| | MNMTPRRPGPTRKHYQPYAPPRDFAAYRSKRGRKKLLYIFKQP |
| | FMRPVQTTQEEDGCSCRFPEEEEGGCELRVKFSRSADAPAYKQ |
| | GQNQLYNELNLGRREEYDVLDKRRGRDPEMGGKPRRKNPQEG |
| | LYNELQKDKMAEAYSEIGMKGERRRGKGHDGLYQGLSTATKD |
| | TYDALHMQALPPR (SEQ ID NO: 21953) |
| scFv | MEFGLSWLFLVAILKGVQCQVQLRESGPSLVKPSQTLSLTCTVS |
| | GFSLTSYGVHWVRQPPGKGLEWLGVIWTGGTTNYNTALMSRL |
| | SITKDNSKSQVFLKMNSLQTDDTAIYYCARDGDSYNREAWFAY |
| | WGQGTLVTVSAGGGGSGGGGSGGGGSGGQIVLTQSPAVISASP |
| | GEKVILTCSASSSVSYMHWFQQKPGTSPKLWIYGTSNLASGVP |
| | ARFSGGGSGTSYSLTISRMEAEDAATYYCQQRSRYPFTFGSGTK |
| | LEIK (SEQ ID NO: 21954) |
| Full length | QVQLRESGPSLVKPSQTLSLTCTVSGFSLTSYGVHWVRQPPGK |
| HLA-E | GLEWLGVIWTGGTTNYNTALMSRLSITKDNSKSQVFLKMNSLQ |
| activator | TDDTAIYYCARDGDSYNREAWFAYWGQGTLVTVSAGGGGSG |
| receptor | GGGSGGGGSGGQIVLTQSPAVISASPGEKVILTCSASSSVSYMH |
| | WFQQKPGTSPKLWIYGTSNLASGVPARFSGGGSGTSYSLTISRM |
| | EAEDAATYYCQQRSRYPFTFGSGTKLEIKTTTPAPRPPTPAPTIA |
| | SQPLSLRPEACRPAAGGAVHTRGLDFACDFWVLVVVGGVLAC |
| | YSLLVTVAFIIFWVRSKRSRLLHSDYMNMTPRRPGPTRKHYQP |
| | YAPPRDFAAYRSKRGRKKLLYIFKQPFMRPVQTTQEEDGCSCR |
| | FPEEEEGGCELRVKFSRSADAPAYKQGQNQLYNELNLGRREEY |
| | DVLDKRRGRDPEMGGKPRRKNPQEGLYNELQKDKMAEAYSEI |
| | GMKGERRRGKGHDGLYQGLSTATKDTYDALHMQALPPR (SEQ |
| | ID NO: 22016) |
| Full length | SEQ ID NO: 22017 |
| HLA-E | |
| activator | |
| receptor | |
| (DNA | |
| sequence) | |
| VL domain | SEQ ID NO: 7 |
| VH domain | SEQ ID NO: 8 |
| CD8α hinge | SEQ ID NO: 219 |
| CD28 TM | SEQ ID NO: 232 |
| domain | |
| CD28 co- | SEQ ID NO: 243 |
| stimulatory | |
| domain | |
| 4-1BB | SEQ ID NO: 487 |
| costimulatory | |
| domain | |
| CD3z | SEQ ID NO: 238 |
| intracellular | |
| domain | |
| Intracellular | SEQ ID NO: 489 |
| domain | |

### Example 2: Activity of an HLA-E Chimeric Antigen Receptor in Jurkat Cells

An HLA-E scFv comprising a light chain variable region of SEQ ID NO: 7 and a heavy chain variable region of SEQ ID NO: 8 was used to generate a CAR with a CD8 hinge, CD28 transmembrane domain, and CD28/41BB/CD3 intracellular domain (third generation CAR) which was used as an activator responsive to HLA-E.

HLA-E was knocked out in HeLa cells as described above, to generate an HLA-E null target cell line.

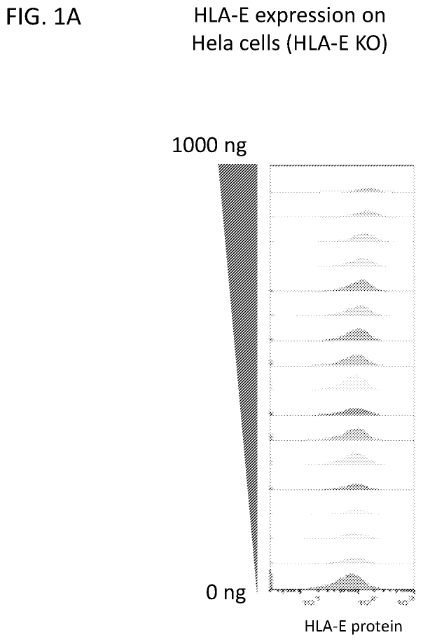

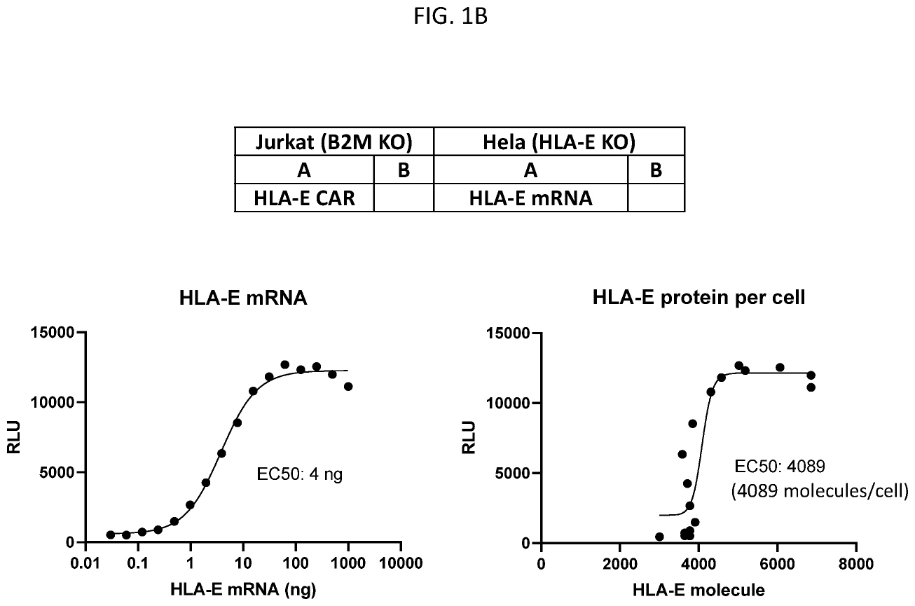

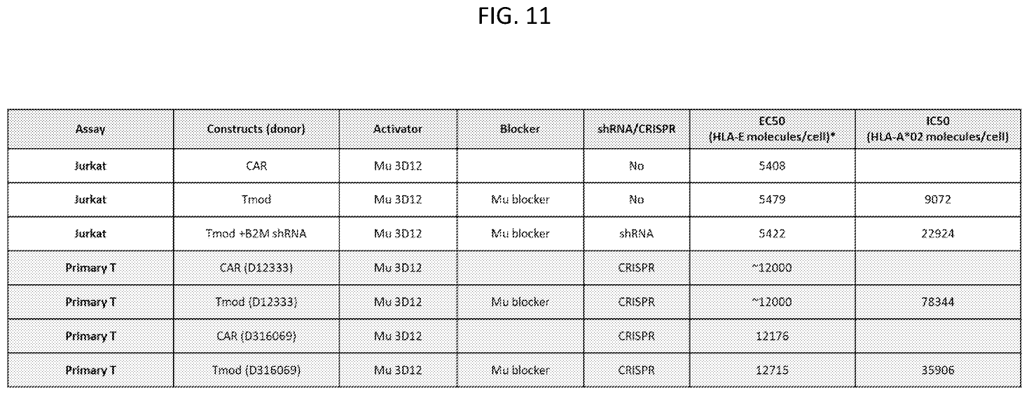

In order to determine the sensitivity of the HLA-E CAR, HLA-E mRNA at a series of amounts ranging from 0.01 to 10,000 ng was used to transfect HLA-E null HeLa cells (12,000 cells/well) in a final volume of 20 mL in 384-well plates (Corning 3570). HeLa cells were co-cultured with an HLA-E CAR expressing Jurkat cells that also expressed the NFAT-Luciferase reported. The level of activation was quantified by the intensity of luminescence upon Jurkat activation (FIG. 11n). The HLA-E density on HeLa cells at each mRNA dosage was determined by flow cytometry using Qifikit, and is shown in FIG. 1A.

### Example 3: Inhibition of HLA-E CAR by an HLA-A*02 Inhibitory Receptor in Jurkat Cells

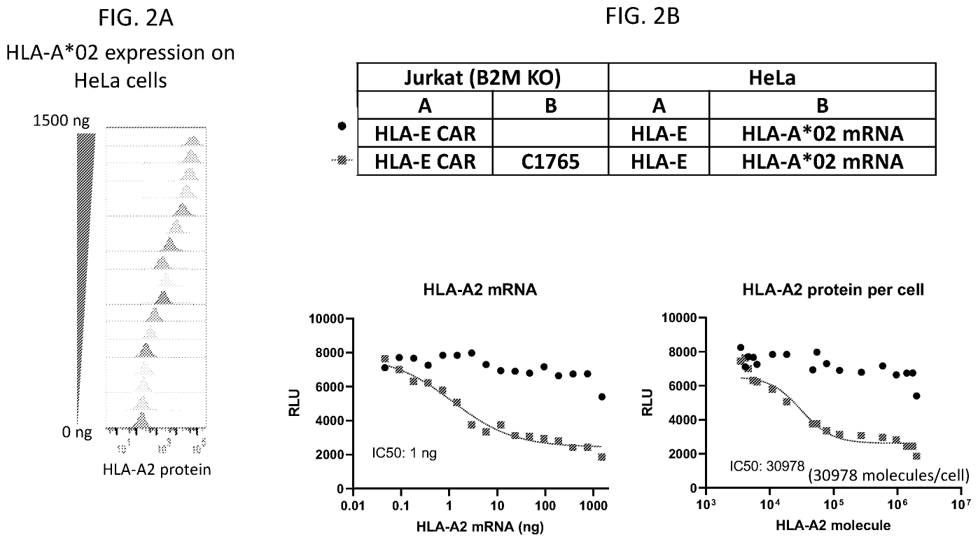

The degree of blocking of HLA-E CAR activity by an HLA-A*02 inhibitory receptor was determined (which has an HLA-A*02 antigen binding domain comprising SEQ ID NO: 63 and a LILRB1 hinge, transmembrane and intracellular domains, SEQ ID NO: 264. Wild-type HeLa cells, which are HLA-E+, were transfected with HLA-A*02 mRNA at a series of amounts as shown in FIG. 2A. HeLa cells expressing different levels of HLA-A*02 were cocultured with NFAT-Luciferase Jurkat cells expressing either the HLA-E CAR alone, or the HLA-E CAR in combination with the HLA-A*02 inhibitory receptor. The level of blocking was quantified by the decrease of intensity of luminescence of the Jurkat cells, as shown in FIG. 2B. HLA-A*02 density on HeLa cells at each mRNA amount was determined by flow cytometry using Qifikit (FIG. 2A).

### Example 4: Sensitivity of the HLA-E CAR Activator in Primary T Cells

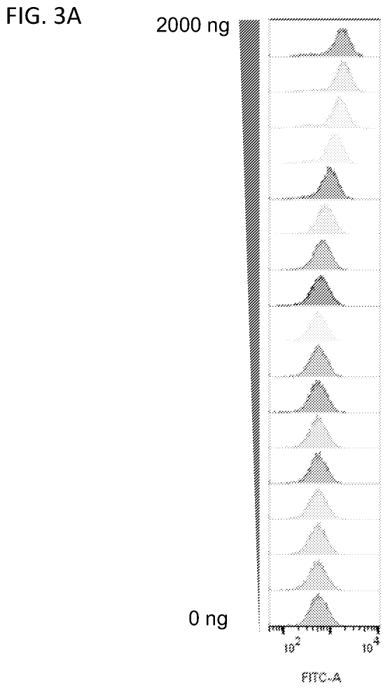

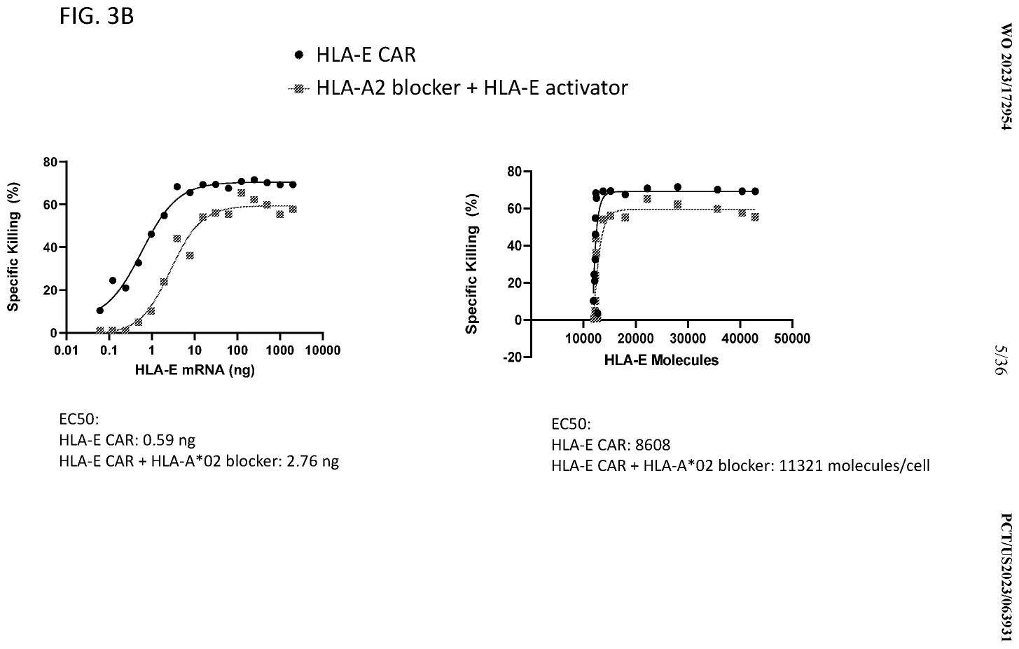

Specific killing was measured to determine sensitivity of HLA-E activator CAR. HLA-E KO cells expressing RFP were transfected with a series of HLA-E mRNA (from 2000 ng to 0 ng, in 2× dilutions, FIG. 3A). Histograms of HLA-E surface expression, as measured by flow cytometry, confirmed that a series of levels of HLA-E expression were generated depending upon the amount of mRNA used in transfection, with 2000 ng mRNA in transfection generating the highest amount of HLA-E expression. Primary T cells expressing the HLA-E CAR and the HLA-E CAR plus HLA-A2 blocker were co-cultured with target HeLa cells. Images of survivors of target cells were acquired and curves of specific killing were fitted to obtain EC50. HLA-E molecules per HeLa cell were measured using QIFIKIT® (quantitative analysis kit from Agilent®). Both the HLA-E CAR and CT 1188 show a similar EC50, ˜10,000 HLA-E molecules. The results are shown in FIG. 3B.

### Example 5: Sensitivity of the HLA-E CAR/HLA-A*02 Blocker Pair in Primary T Cells

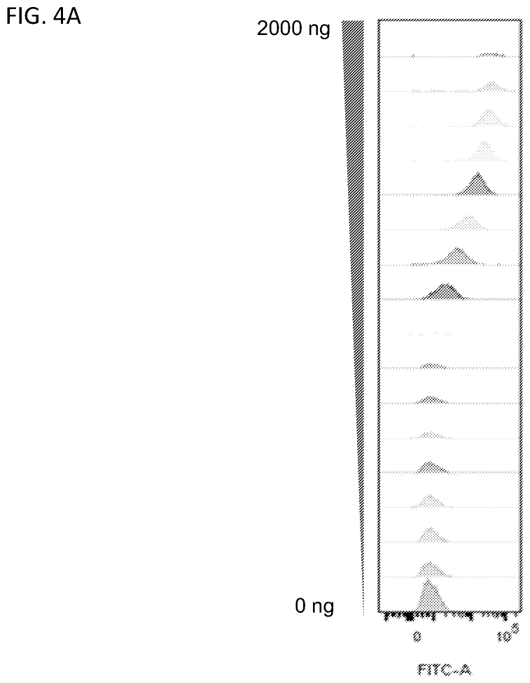

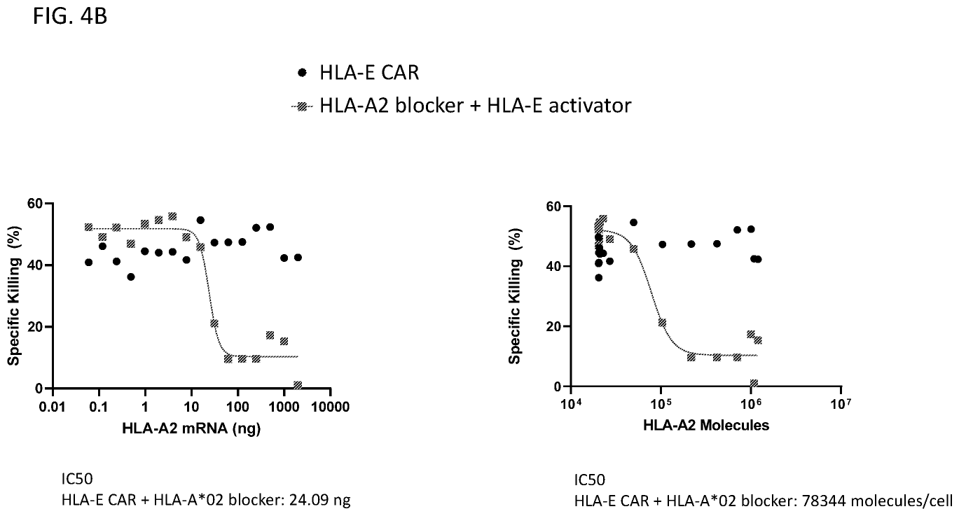

HeLa cells (endogenously negative for HLA-A*02, and positive for HLA-E) expressing RFP were transfected with a series of HLA-A2 mRNA (from 2000 ng to 0 ng in a 2× dilution), as shown in FIG. 4A. Expression of surface HLA-A*02, as measured by flow cytometry, was plotted. The histograms confirm that the highest levels of HLA-A*02 expression were seen when 2000 ng mRNA was used in transfection. FIG. 4B shows the specific killing by primary T cells of HeLa cells transfected with variable amounts of HLA-A*02 as shown in FIG. 4A. Specific killing was assayed as described in Example 4. Primary T cells that were transduced with the HLA-E activator CAR, or the HLA-E CAR in combination with the HLA-A*02 LIR1 inhibitor receptor. % specific killing was plotted against the amount of HLA-A*02 mRNA used in HeLa cell transfection (FIG. 4B, left) or total HLA-A*02 molecules (FIG. 4B, right plot), and curves were fitted to obtain IC50 (˜80,000 HLA-A2 molecules) for the HLA-A*02 inhibitory receptor. The number of HLA-A*02 molecules per HeLa cell was measured using an Agilent® QIFIKIT® quantitative analysis kit.

### Example 6: Activity of HLA-E CAR when Paired with HLA-A*03, HLA-A*011 and HLA-B*07 Inhibitory Receptors in Jurkat Cells

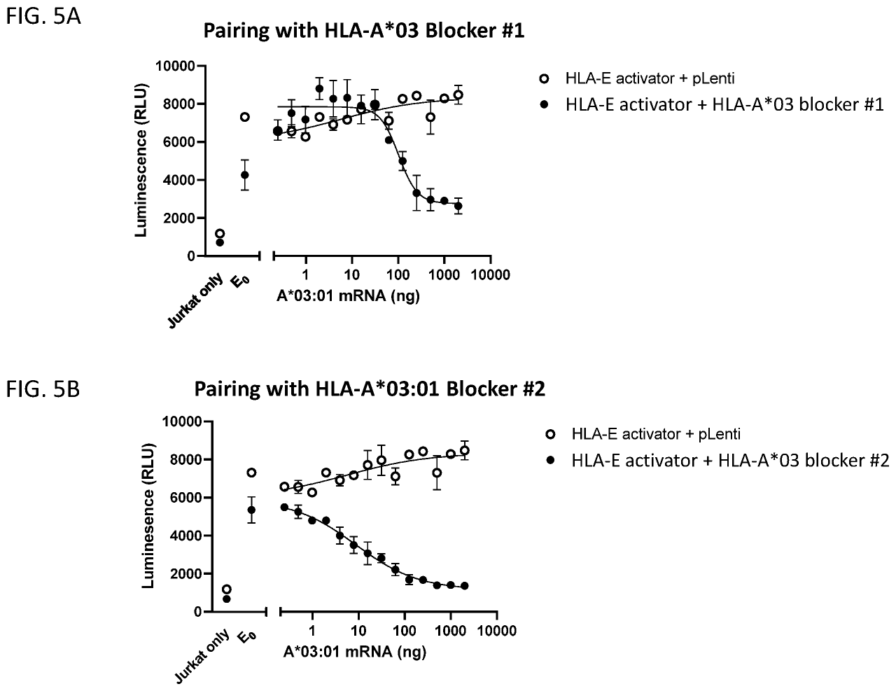

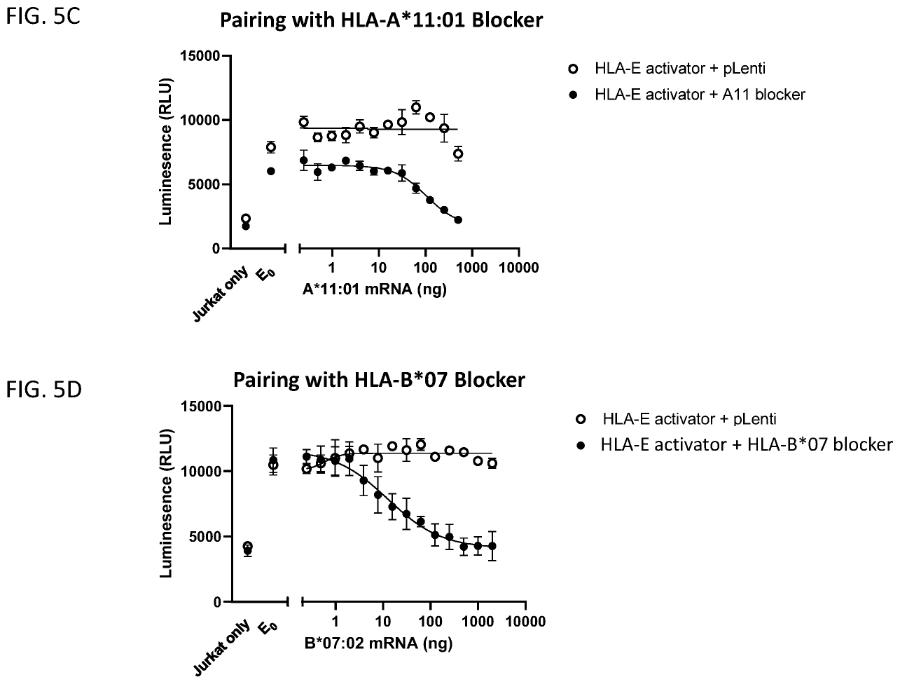

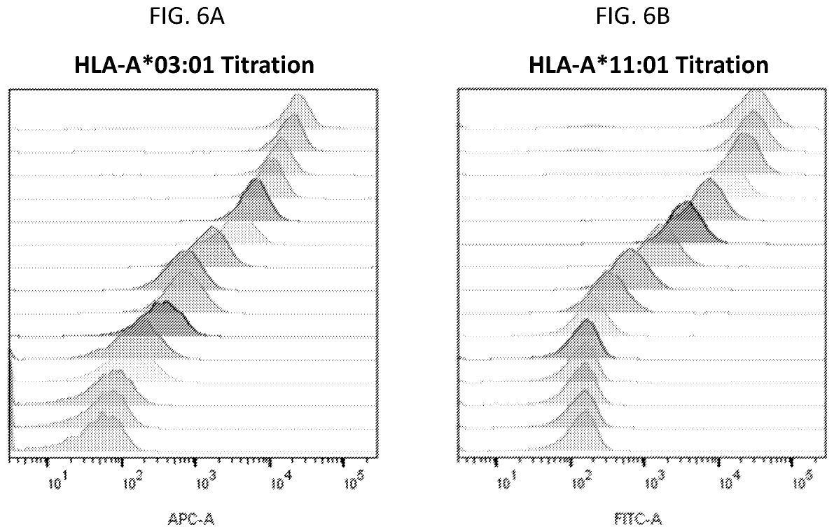

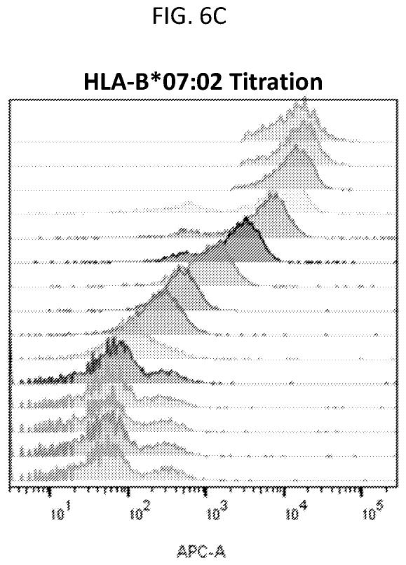

Jurkat NFAT luciferase (JNL) cells in which β-2 microglobulin (B2M) had been knocked out using CRISPR/Cas9 as described above (B2M KO) were used to assay Jurkat cell inhibition mediated by HLA-A*03, HLA-A*011 and HLA-B*07 LIR1 inhibitory receptors. B2M KO Jurkat NFAT luciferase cells were transiently transfected with HLA-E CAR and a negative control vector (open circles, FIGS. 5A-5D), or with the HLA-E CAR and either an HLA-A*03:01, HLA-A*11:01, or HLA-B*07:02 inhibitor receptor (closed circles, FIGS. 5A-5D, sequences provided in Table 12 below). Jurkat cell inhibition by the inhibitor receptors, in the presence of the HLA-E CAR and its cognate ligand, was measured using an HLA-A*03:01, HLA-A*11:01, or HLA-B*07:02 mRNA titration assay. In brief, HeLa cells were transfected with serially-diluted amounts of mRNA encoding HLA-A*03:01, HLA-A*11:01, or HLA-B*07:02 mRNA, as shown in FIGS. 6A-6C, respectively. The functional response of the Jurkat cells was assessed after a 6-hour co-culture with transfected HeLa cell, using 5,000 B2M KO JNL cells and 5,000 HeLa cells per assay condition. The results, as seen in FIGS. 5A-5D, show that HLA-A*03:01, HLA-A*11:01, and HLA-B*07:02 inhibitor receptors can all block activation of the HLA-E CAR in the presence of the titrated corresponding ligand expressed by HeLa cells. However, in the absence of the inhibitor receptor, the HLA-E CAR mediates robust activation of Jurkat cells (FIGS. 5A-5D, open circles).

TABLE 12
HLA-A*02, HLA-A*03, HLA-A*11:01 and HLA-B*07 inhibitor receptor
sequences

| Construct | Sequence |
|---|---|
| HLA-A*03 | MDMRVPAQLLGLLLLWLRGARCEVKLEESGGGLVQPGGSMKLSCVAS |
| LIR1 blocker | GFTFSNYWMNWVRQSPEKGLEWVAEIRLKSTNYATHYAESVKGRFTI |
| #1, Protein | SRDDSKSSVYLQMNNLRAEDTGIYYCTTLITPDYWGQGTTLTVSSGG |
| sequence | GGSGGGGSGGGGSGGDIVMTQSHKFMSTSVGDRVSITCKASQDVSTT |
| | VAWYQQKPGQSPKLLIYSASYRYTGVPDRFTGSGSGTDFTFTISSVQ |
| | AEDLAVYYCQQHYSTPPTFGGGTKLEIKYGSQSSKPYLLTHPSDPLE |
| | LVVSGPSGGPSSPTTGPTSTSGPEDQPLTPTGSDPQSGLGRHLGVVI |
| | GILVAVILLLLLLLLLFLILRHRRQGKHWTSTQRKADFQHPAGAVGP |
| | EPTDRGLQWRSSPAADAQEENLYAAVKHTQPEDGVEMDTRSPHDEDP |
| | QAVTYAEVKHSRPRREMASPPSPLSGEFLDTKDRQAEEDRQMDTEAA |
| | ASEAPQDVTYAQLHSLTLRREATEPPPSQEGPSPAVPSIYATLAIH |
| | (SEQ ID NO: 21945) |
| HLA-A*03 | SEQ ID NO: 21946 |
| LIR1 blocker | |
| #1, DNA | |
| sequence | |
| HLA- | MDMRVPAQLLGLLLLWLRGARCQVQLVQSGAEVKKPGASVKVSCKAS |
| A*03:01 LIR1 | GYTFTSYGISWVRQAPGQGLEWMGWISAYNGNTNYAQKLQGRVTMTT |
| blocker #2, | DTSTSTAYMELRSLRSDDTAVYYCARETPPSLGAFDIWGQGTMVTVS |
| Protein | SGGGGSGGGGSGGGGSGGQSALTQPPSASGSPGQSVTISCTGTSSDV |
| sequence | GGYNYVSWYQQHPGKAPKLMIYEVSKRPSGVPDRESGSKSGNTASLT |
| | VSGLQAEDEADYYCSSYAGSNNWVFGGGTKLTVLYGSQSSKPYLLTH |
| | PSDPLELVVSGPSGGPSSPTTGPTSTSGPEDQPLTPTGSDPQSGLGR |
| | HLGVVIGILVAVILLLLLLLLLFLILRHRRQGKHWTSTQRKADFQHP |
| | AGAVGPEPTDRGLQWRSSPAADAQEENLYAAVKHTQPEDGVEMDTRS |
| | PHDEDPQAVTYAEVKHSRPRREMASPPSPLSGEFLDTKDRQAEEDRQ |
| | MDTEAAASEAPQDVTYAQLHSLTLRREATEPPPSQEGPSPAVPSIYA |
| | TLAIH |
| | (SEQ ID NO: 21947) |
| HLA- | SEQ ID NO: 21948 |
| A*03:01 LIR1 | |
| blocker #2, | |
| DNA | |
| sequence | |
| HLA-A*11 | MDMRVPAQLLGLLLLWLRGARCEVQLVESGGGLVQPGGSLRLSCAAS |
| LIR1 blocker, | GFTFSSYWMHWVRQAPGKGLVWVSRINSDGSSTSYADSVKGRFTISR |
| Protein | DNAKNTLYLQMNSLRAEDTAVYYCCLGVLLYNWEDPWGQGTLVTVSS |
| sequence | GGGGSGGGGSGGGGSGGDIQMTQSPSSLSASVGDRVTITCRASQSIS |
| | SYLNWYQQKPGKAPKLLIYAASSLQSGVPSRESGSGSGTDFTLTISS |
| | LQPEDFATYYCQQSYSTPLTFGGGTKVEIKYGSQSSKPYLLTHPSDP |
| | LELVVSGPSGGPSSPTTGPTSTSGPEDQPLTPTGSDPQSGLGRHLGV |
| | VIGILVAVILLLLLLLLLFLILRHRRQGKHWTSTQRKADFQHPAGAV |
| | GPEPTDRGLQWRSSPAADAQEENLYAAVKHTQPEDGVEMDTRSPHDE |
| | DPQAVTYAEVKHSRPRREMASPPSPLSGEFLDTKDRQAEEDRQMDTE |
| | AAASEAPQDVTYAQLHSLTLRREATEPPPSQEGPSPAVPSIYATLAI |
| | H |
| | (SEQ ID NO: 21949) |
| HLA-A*11 | SEQ ID NO: 21950 |
| LIR1 blocker, | |
| DNA | |
| sequence | |
| HLA-B*07 | MDMRVPAQLLGLLLLWLRGARCDVQLQESGPDLVKPSQSLSLTCTVT |
| LIR1 blocker, | GYSITSGYSWHWIRQFPRNKLEWMGYIHFSGSTHYHPSLKSRISITR |
| Protein | DTSKNQFFLQLNSVTTEDTATYYCARGGVVSHYAMDCWGQGTSVTVS |
| sequence | SGGGGSGGGGSGGGGSGGDIQMTQSPASLSVSVGETVTITCRASENI |
| | YSNLAWYQQKQGKSPHLLVYAATYLPDGVPSRESGSGSGTQYSLKIN |
| | SLQSEDFGSYYCQHFWVTPYTFGGGTKVEIKYGSQSSKPYLLTHPSD |
| | PLELVVSGPSGGPSSPTTGPTSTSGPEDQPLTPTGSDPQSGLGRHLG |
| | VVIGILVAVILLLLLLLLLFLILRHRRQGKHWTSTQRKADFQHPAGA |
| | VGPEPTDRGLQWRSSPAADAQEENLYAAVKHTQPEDGVEMDTRSPHD |
| | EDPQAVTYAEVKHSRPRREMASPPSPLSGEFLDTKDRQAEEDRQMDT |
| | EAAASEAPQDVTYAQLHSLTLRREATEPPPSQEGPSPAVPSIYATLA |
| | IH |
| | (SEQ ID NO: 21951) |
| HLA-B*07 | SEQ ID NO: 21952 |
| LIR1 blocker, | |
| DNA | |
| sequence | |
| HLA-A*02 | DVLMTQTPLSLPVSLGDQASISCRSSQSIVHSNGNTYLEWYLQ |
| LIR1 blocker, | KPGQSPKLLIYKVSNRFSGVPDRFSGSGSGTDFTLKISRVEAED |
| protein | LGVYYCFQGSHVPRTSGGGTKLEIKGGGGSGGGGSGGGGSGG |
| sequence | QVQLQQSGPELVKPGASVRISCKASGYTFTSYHIHWVKQRPGQ |
| | GLEWIGWIYPGNVNTEYNEKFKGKATLTADKSSSTAYMHLSS |
| | LTSEDSAVYFCAREEITYAMDYWGQGTSVTVSSYGSQSSKPYL |
| | LTHPSDPLELVVSGPSGGPSSPTTGPTSTSGPEDQPLTPTGSDPQ |
| | SGLGRHLGVVIGILVAVILLLLLLLLLFLILRHRRQGKHWTSTQ |
| | RKADFQHPAGAVGPEPTDRGLQWRSSPAADAQEENLYAAVK |
| | HTQPEDGVEMDTRSPHDEDPQAVTYAEVKHSRPRREMASPPS |
| | PLSGEFLDTKDRQAEEDRQMDTEAAASEAPQDVTYAQLHSLT |
| | LRREATEPPPSQEGPSPAVPSIYATLAIH (SEQ ID NO: 21965) |
| murine HLA- | DVLMTQTPLSLPVSLGDQASISCRSSQSIVHSNGNTYLEWYLQ |
| A*02 LIR1 | KPGQSPKLLIYKVSNRFSGVPDRFSGSGSGTDFTLKISR VEAED |
| blocker, | LGVYYCFQGSHVPRTSGGGTKLEIKGGGGSGGGGSGGGGSGG |
| protein | QVQLQQSGPELVKPGASVRISCKASGYTFTSYHIHWVKQRPGQ |
| sequence | GLEWIGWIYPGNVNTEYNEKFKGKATLTADKSSSTAYMHLSS |
| | LTSEDSAVYFCAREEITYAMDYWGQGTSVTVSSYGSQSSKPYL |
| | LTHPSDPLELVVSGPSGGPSSPTTGPTSTSGPEDQPLTPTGSDPQ |
| | SGLGRHLGVVIGILVAVILLLLLLLLLFLILRHRRQGKHWTSTQ |
| | RKADFQHPAGAVGPEPTDRGLQWRSSPAADAQEENLYAAVK |
| | HTQPEDGVEMDTRSPHDEDPQAVTYAEVKHSRPRREMASPPS |
| | PLSGEFLDTKDRQAEEDRQMDTEAAASEAPQDVTYAQLHSLT |
| | LRREATEPPPSQEGPSPAVPSIYATLAIH (SEQ ID NO: 22012) |
| murine HLA- | SEQ ID NO: 22013 |
| A*02 LIR1 | |
| blocker, DNA | |
| sequence | |
| humanized | QVQLVQSGAEVKKPGSSVKVSCKASGYTFTSYHIHWVRQAPG |
| HLA-A*02 | QGLEWIGWIYPGNVNTEYNEKFKGKATITADESTNTAYMELS |
| LIR1 blocker, | SLRSEDTAVYYCAREEITYAMDYWGQGTLVTVSSGGGGSGG |
| protein | GGSGGGGSGGDIQMTQSPSTLSASVGDRVTITCRSSQSIVHSNG |
| sequence | NTYLEWYQQKPGKAPKLLIYKVSNRFSGVPARFSGSGSGTEFT |
| | LTISSLQPDDFATYYCFQGSHVPRTFGQGTKVEVKYGSQSSKP |
| | YLLTHPSDPLELVVSGPSGGPSSPTTGPTSTSGPEDQPLTPTGSD |
| | PQSGLGRHLGVVIGILVAVILLLLLLLLLFLILRHRRQGKHWTS |
| | TQRKADFQHPAGAVGPEPTDRGLQWRSSPAADAQEENLYAA |
| | VKHTQPEDGVEMDTRSPHDEDPQAVTYAEVKHSRPRREMASP |
| | PSPLSGEFLDTKDRQAEEDRQMDTEAAASEAPQDVTYAQLHS |
| | LTLRREATEPPPSQEGPSPAVPSIYATLAIH (SEQ ID NO: 22014) |
| humanized | SEQ ID NO: 22015 |
| HLA-A*02 | |
| LIR1 blocker, | |
| DNA | |
| sequence | |

### Example 7: HLA-E Tmod Cells Exhibit Potent, Selective Cytotoxicity In Vitro and In Vivo

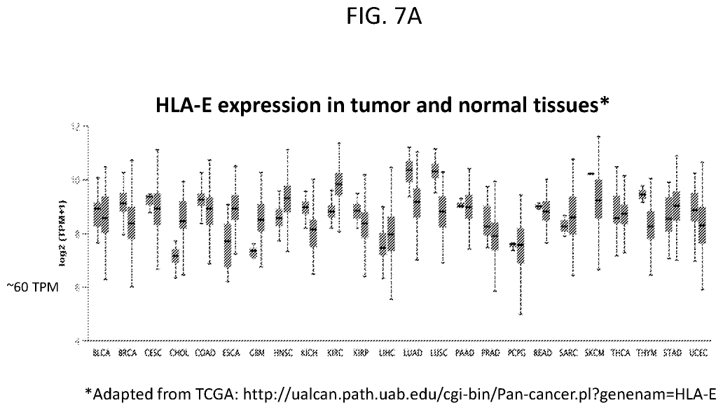

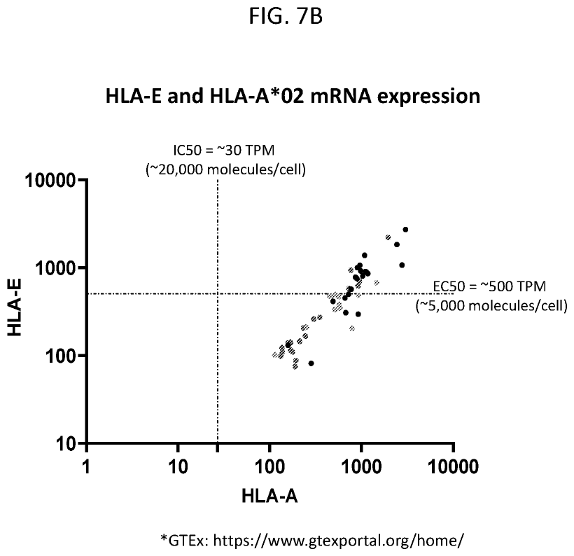

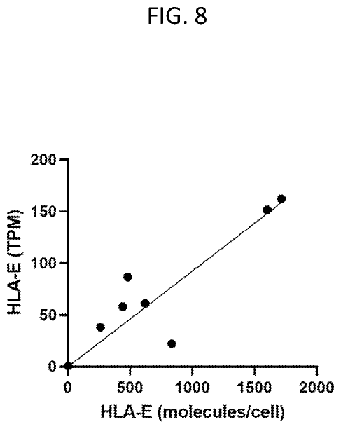

The HLA-E expression in tumor and normal tissues was determined as shown in FIG. 7A. Left bars on each column are tumor samples with median and quartile delimiters while right bars on each column are normal adjacent tissues. Abbreviations are tumor types according to the TCGA convention. HLA-E expression is also correlate with HLA-A expression (FIG. 7B). Red dots represent core battery organs, black dots other organs, and orange dots supplementary to the core battery organs. In order to determine the HLA-E surface protein levels (molecules/cell), mRNA levels (TPM) were converted using a QIFIKIT® quantitative analysis kit (FIG. 8).

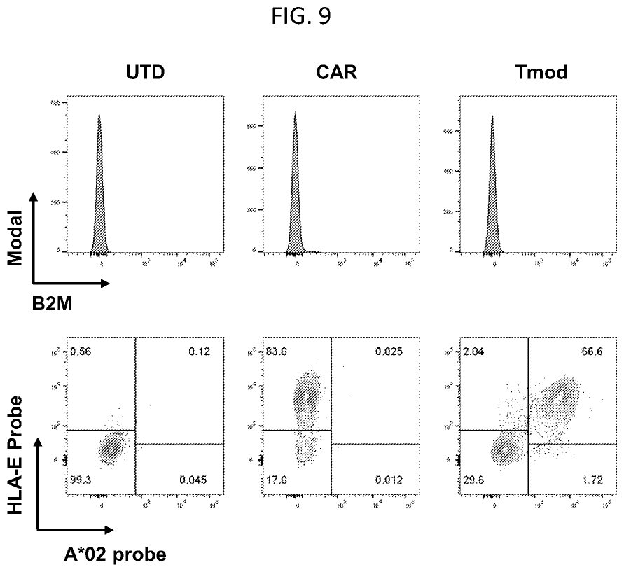

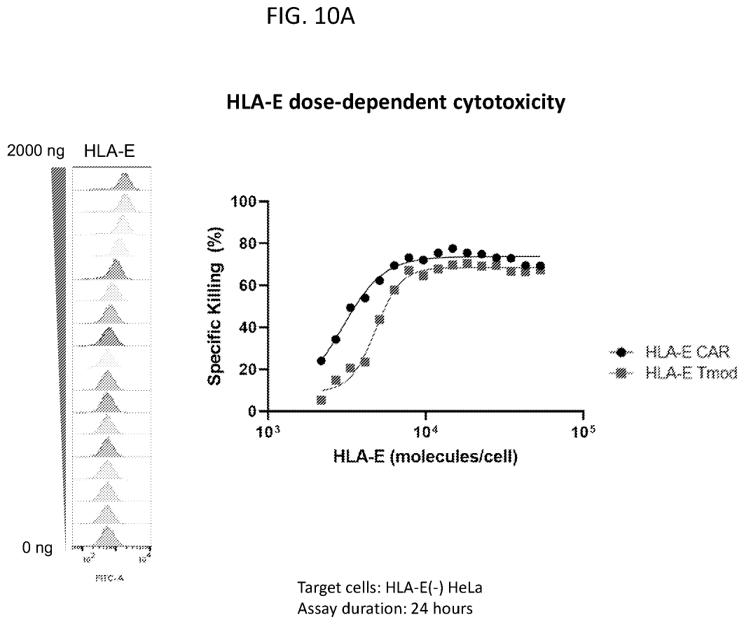

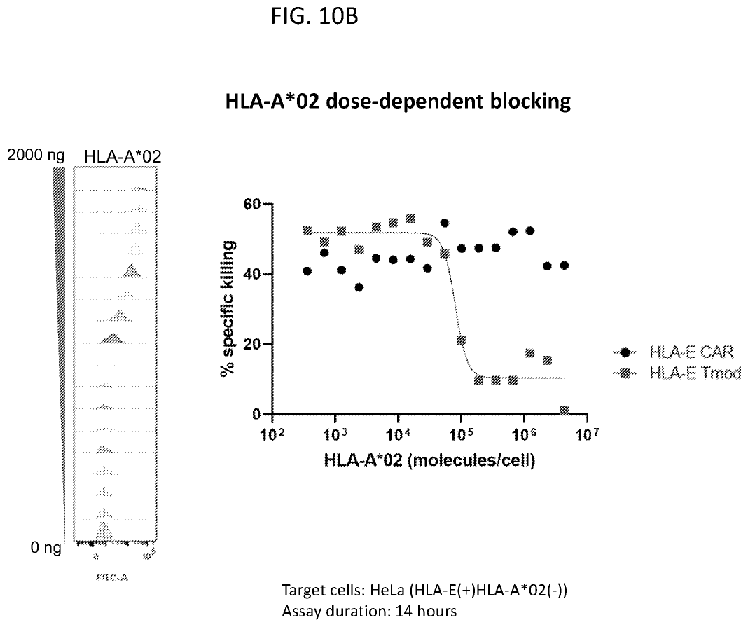

The histograms confirm the expression of HLA-E CAR and Tmod modules in primary T cells (FIG. 9). Briefly, B2M null 4809 donor cells were transduced with CAR or Tmod constructs. HLA-E activator was stained with biotinylated recombinant HLA-E probe tetramerized with PE-streptavidin (HLA-E probe) and HLA-A*02 blockers were stained with biotinylated recombinant HLA-A*02 tetramerized with streptavidin (A2 probe). Specific killing was measured to determine the sensitivity of HLA-E CAR and HLA-E Tmod. HLA-E CAR or HLA-E Tmod expressing primary T cells were treated with HLA-E null HeLa cells (FIG. 10A) or HLA-A*02 null HeLa cells (FIG. 10B) dose-dependently. The results of HLA-E Tmod expressing primary T cells are shown in FIGS. 10A-10B. Dose-response curves were generated by mRNA titration of either HLA-E (FIG. 10A) or A*02 (FIG. 10B). The summary of sensitivity for Jurkat and primary T ell mRNA titartaon assays are shown in FIG. 11.

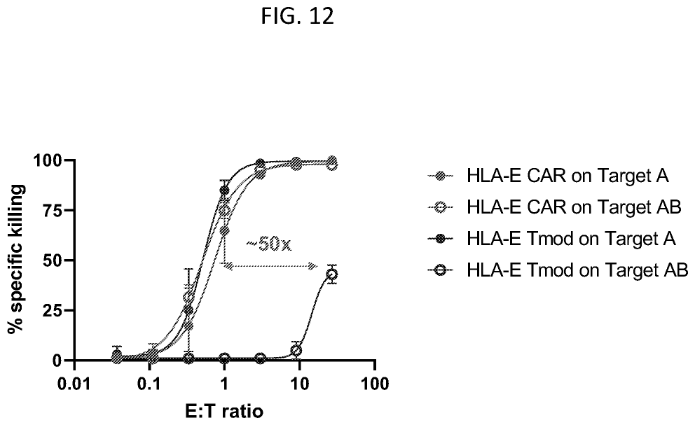

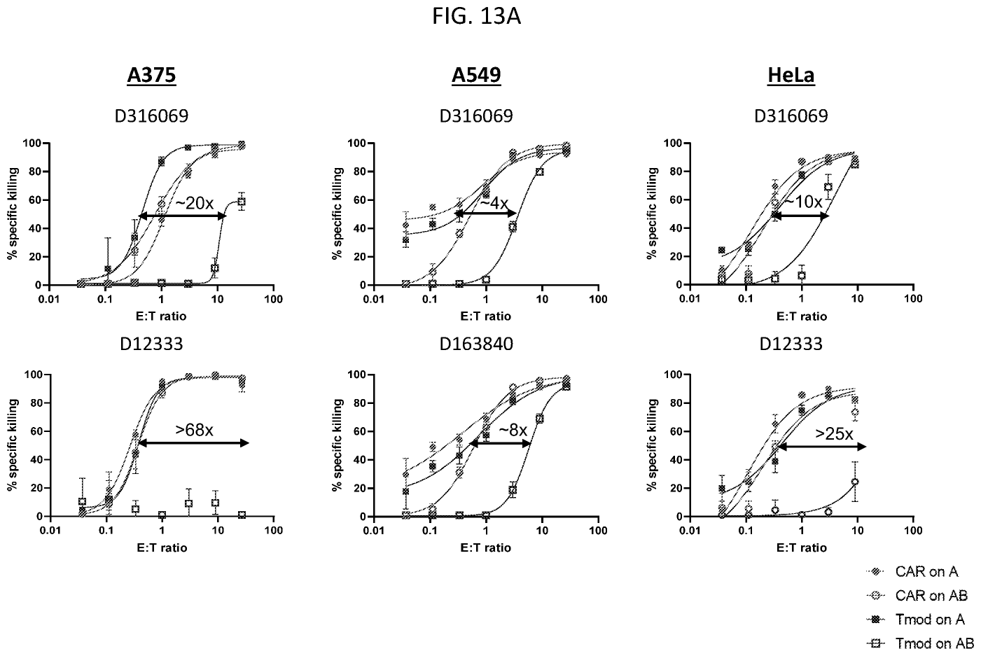

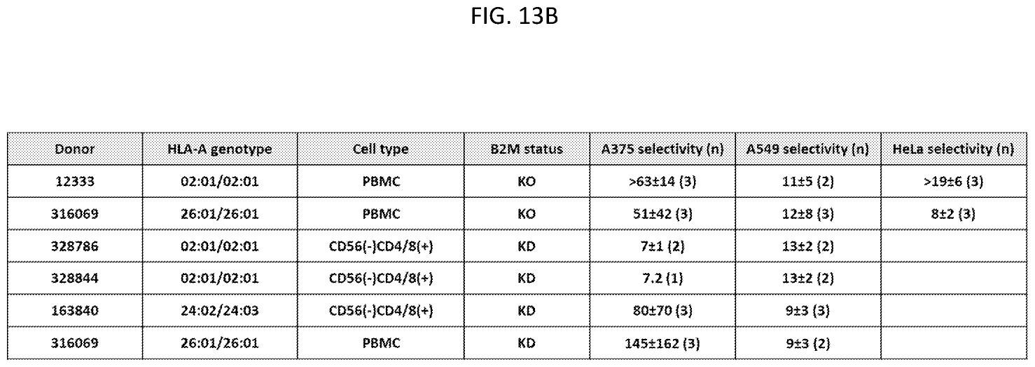

Primary HLA-E CAR or HLA-E Tmod cells were co-cultured with Target A (HLA-A(+) A*02(−)) or Target AB (HLA-A(+) A*02(+)) cells in different ratios for 48 hours and % specific killing was plotted. The result, as seen in FIG. 12, shows that HLA-E Tmod cells display >10× tumor vs. normal selectivity in primary T cell. Additional donors and target cell lines are further tested and the results are shown in FIG. 13A and summarized in FIG. 13B. Donor D12333 is A*02(+). Donors D163840 and D316069 are A*02(−).

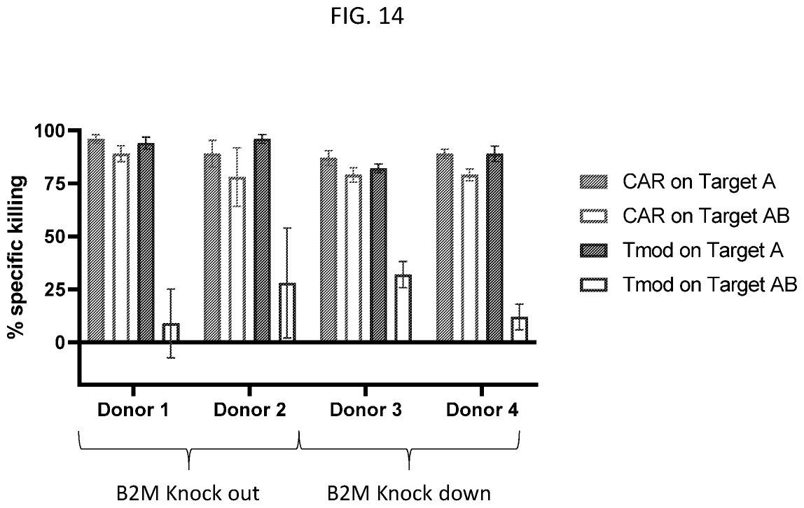

Specific killing was measured to determine tumor-selectivity of HLA-E Tmod cells when tumor and normal target cells are mixed. Target A (HLA-E(+) A*02(−)) and Target AB (HLA-E(+) A*02(+)) cells were mixed before they were co-cultured with HLA-E CAR or HLA-E Tmod cells in various conditions. E:T ratio was 1:1 for donors 1, 2, and 3, and 3:1 for Donor 4. Donors 1, 3, and 4 are A*02(+), and Donor 2 is A*02(−). B2M was knocked out by CRISPR in donors 1 and 2. B2M was knocked down by shRNA in donors 3 and 4 (FIG. 14).

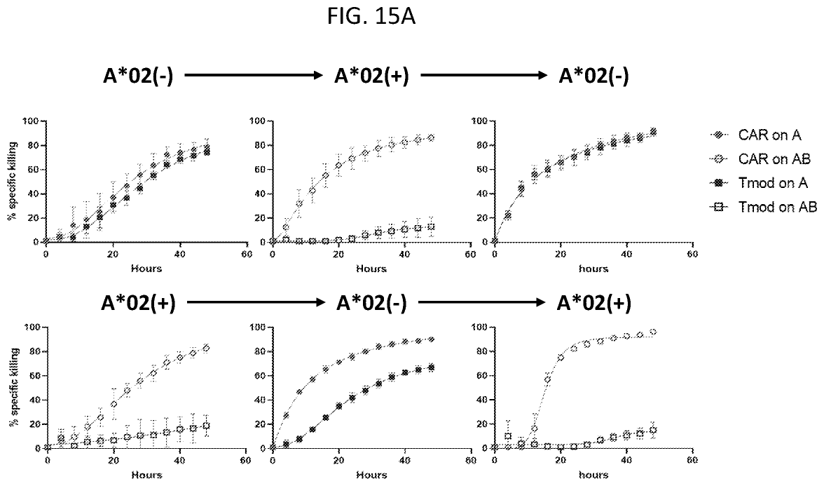

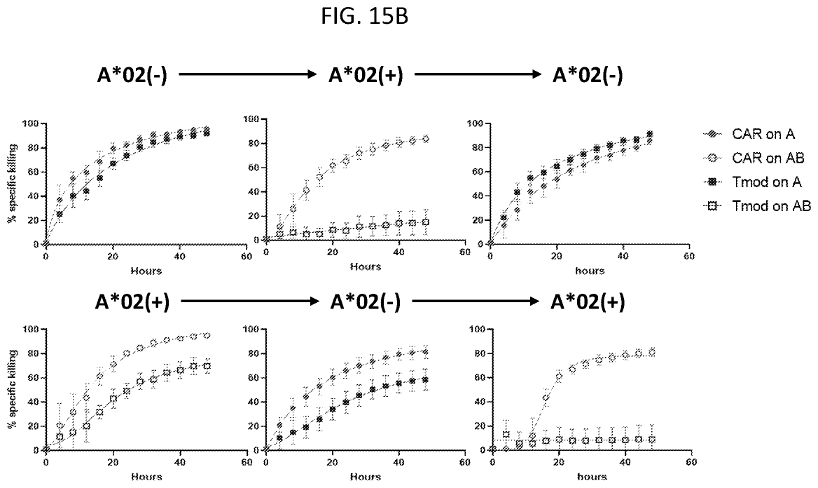

A375 Target A (HLA-E(+) A*02(−)) or Target AB (HLA-E(+) A*02(+)) cells were co-cultured with HLA-E CAR or HLA-E Tmod with the B2M shRNA module cells and measured % specific killing to demonstrated reversible activation (FIG. 15A). Additional D328844 (HLA-A*02(+)) donor cells were used for further experiment (FIG. 15B) and resulted that HLA-E Tmod cells show selective and reversible tumor cell cytotoxicity in serial co-cultures.

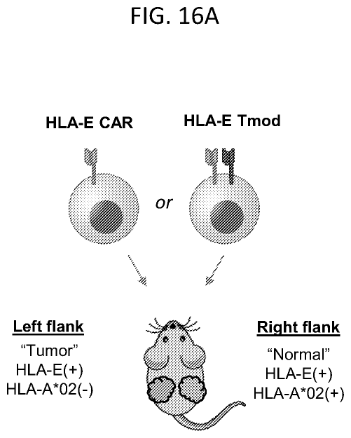

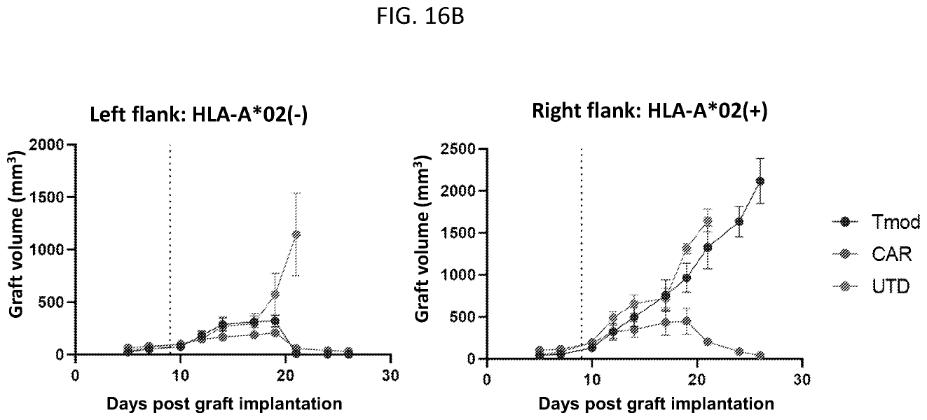

Selective cytotoxicity was observed in vivo xenograft experiments. NSG mice (n=4/cohort) bearing two A375 xenografts, “normal” (HLA-E(+) A*02(+)) on the right flank and “tumor” (HLA-E(+) A*02(−)) on the left flank, were infused with T cells (dose=2E7 cell/mouse) via tail vein injection when the tumors were ˜100 mm3 in volume (FIG. 16A). Xenograft volumes are plotted over time post implant; dotted line indicates day of T-cell injection. Consistent with the in vitro selectivity results, the HLA-E CAR-Ts were unable to distinguish between tumor and normal grafts, while the HLA-E Tmod cells displayed highly specific killing of the tumor (FIG. 16B).

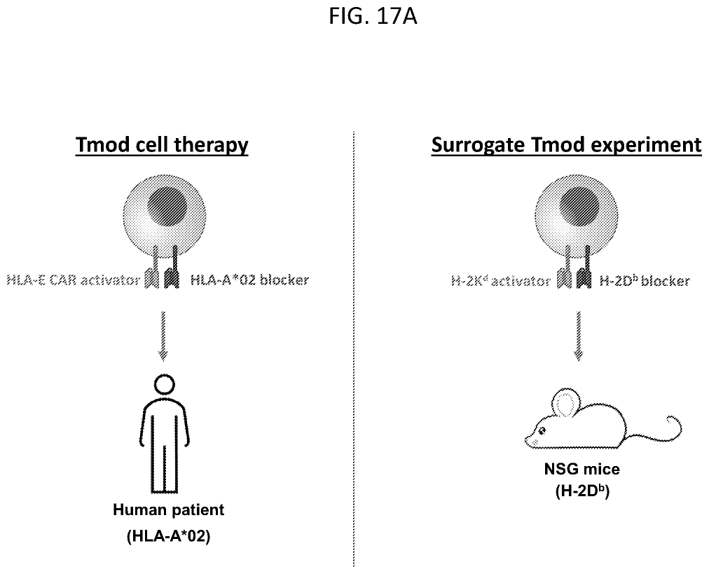

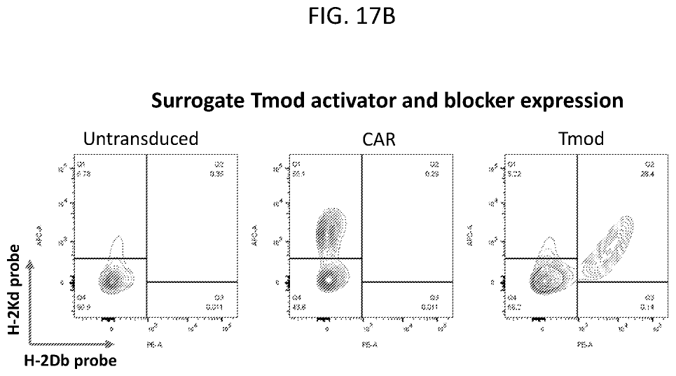

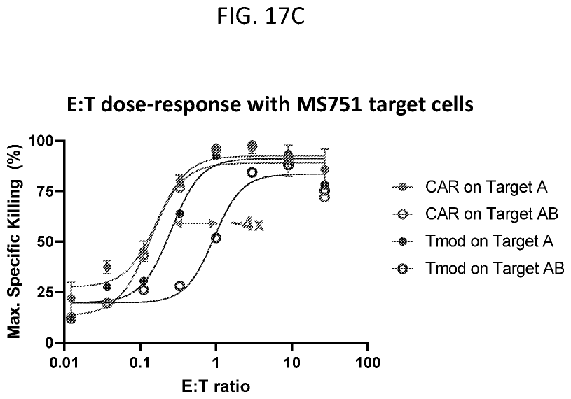

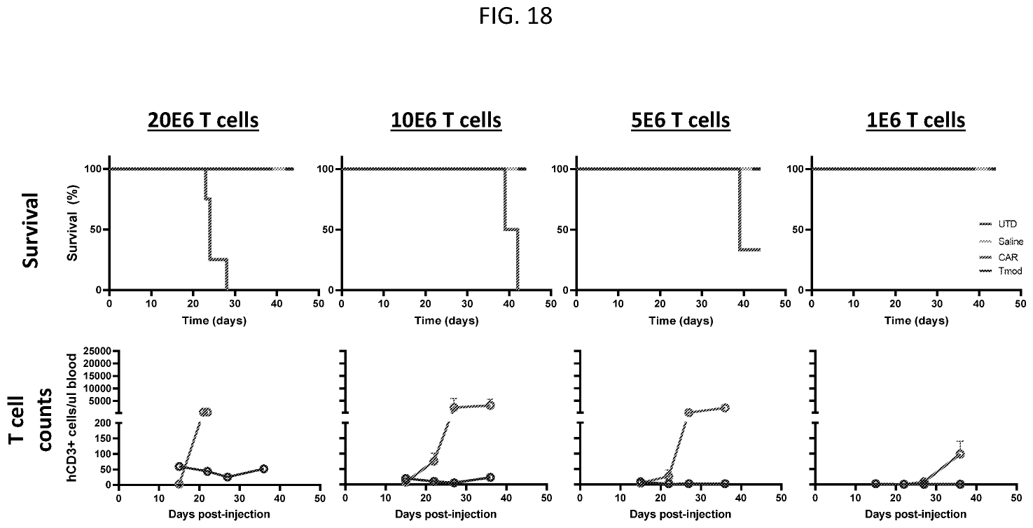

H-2D^{b} scFv was identified that it functioned effectively as a blocker in the context of the LIR-1 backbone. Also H-2K^{d}, a mouse H-2 class I antigen, can be viewed in this context as a mouse ortholog of HLA-E, characterized as an activator, and paired with the H-2D^{b} blocker (FIG. 17A). Expression of surrogate Tmod activator and blocker, as measured by flow cytometry, was plotted (FIG. 17B). Using primary T cell assays in vitro, the H-2K^{d}/D^{b} Tmod system displayed decent potency and a selectivity window of ˜4× compared to the H-2K^{d} CAR alone (FIG. 17C).

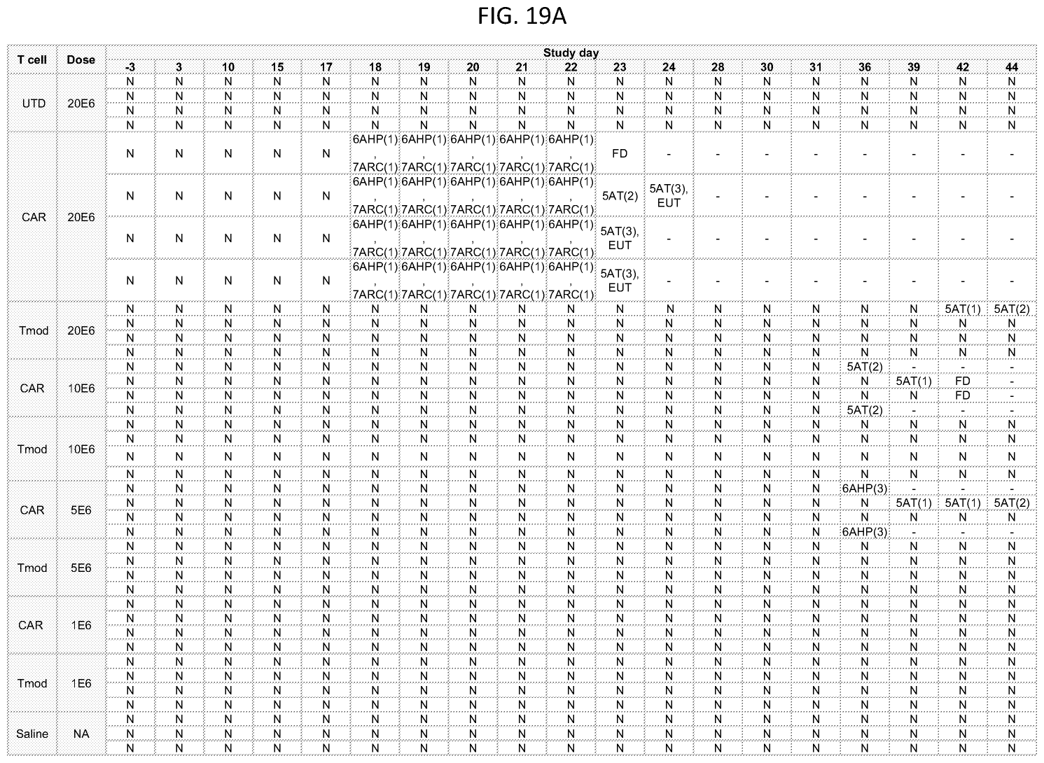

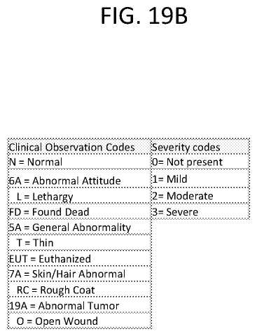

The addition of the H-2D^{b} blocker protected NSG mice significantly from toxicity caused by the H-2K^{d} CAR. At the maximum dose of 20E6 T cells and a dose 2× lowere, H-2K^{d} CAR-Ts killed all mice in the cohort (n=5), whereas H-2K^{d}/D^{b} Tmod cells survived. At a dose 4× below the top dose, half the mice survived in the H-2K^{d} CAR cohort study 6 weeks post T cell infusion. Using human T cell measurements in mouse blood as an indicator of response, Tmod protected the mice over the 20× dose-range tested (FIGS. 19A-19B). Thus, the surrogate HLA-E Tmod construct that targets mouse orthologs protected mice from auto-toxicity supporting the notion that the Tmod system provides a general solution to the problem of tumor selectivity even with tumor antigens that are broadly expressed on normal tissues.

### Example 8: HLA-E Tmod Maintains High Level of Receptor Expression and Function for >4 Weeks; >90% Transduced Cells at the End of Expansion; Reduced Need for T Cell Purification for Allogeneic Treatment

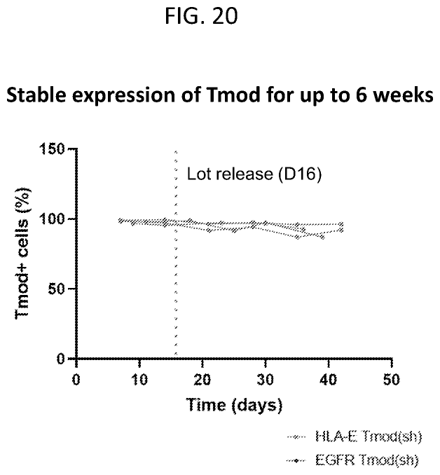

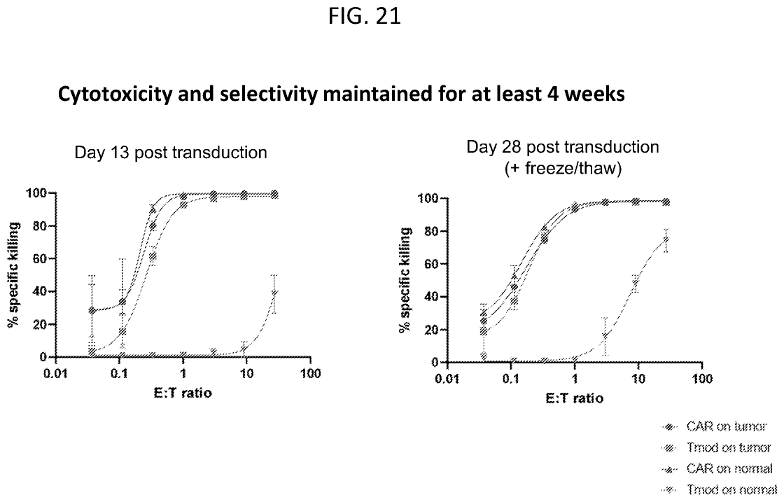

The level of expression of Tmod+ cells were measured up to 6 weeks and % specific killing was plotted in different E:T ratio at day 13 and day 28 post transduction. The results, as seen in FIGS. 20 and 21, show stable expression of Tmod for up to 6 weeks and its cytoxicity and selectivity maintained for at least 4 weeks.

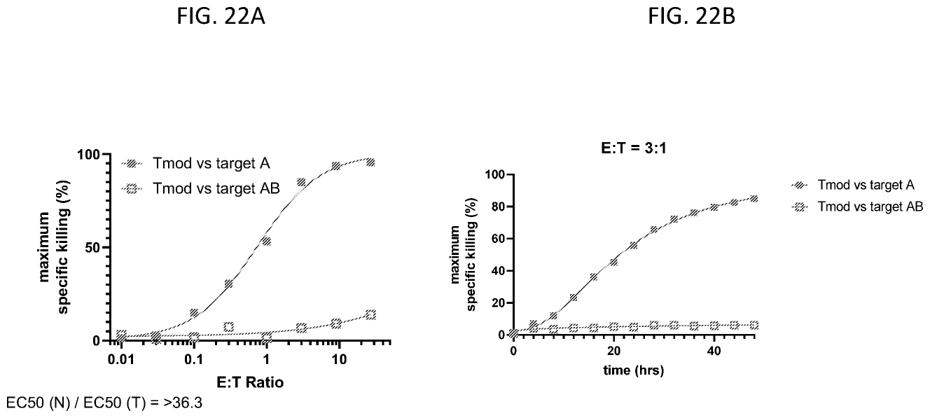

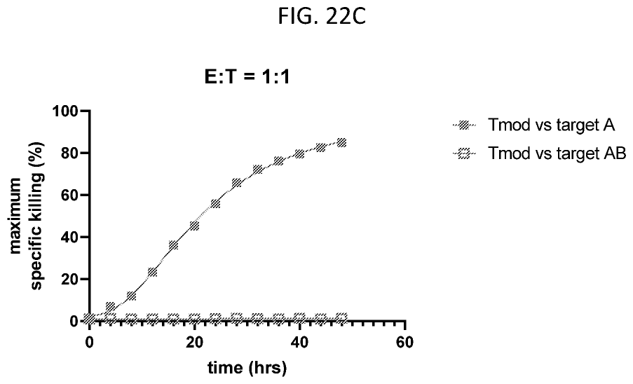

An E:T ratio titration assay was performed. Tmod engineered cells comprising an anti-HLA-E 3D12 activator receptor and HLA-A*02 PA2.1.14 inhibitory recptor were co-cultured with Target A (HLA-E(+) and A*02(−)) or Target AB (HLA-E(+) and A*02(+)) cells at different E:T ratios and % maximum specific killing was plotted (FIG. 22A). The anti-HLA-E/HLA-A*02 T mod engineered cells were evaluated over time at E:T ratio of 3:1 (FIG. 22B) or E:T ratio of 1:1 (FIG. 22C). The activator and inhibitory recpetor were expressed as a single vector in an Alstem virus. Primary T cells from Donor 327596 and A375 target cells were used in the study. The data show robust cytotoxicity and selectivity of anti-HLA-E/HLA-A*02 Tmod engineered cells.

What is claimed is:

1. An immune cell, comprising:
a. an activator receptor comprising an extracellular antigen binding domain specific to a human leukocyte antigen E (HLA-E) antigen expressed by a cancer cell; and
b. an inhibitory receptor comprising an extracellular antigen binding domain specific to a non-target antigen that is not expressed by the cancer cell.

2. The method of claim 1, wherein expression of the non-target antigen is lost in the cancer cell.

3. The method of claim 2, wherein expression of the non-target antigen is lost due to a loss of heterozygosity in the cancer cell.

4. The method of any one of claims 1-3, wherein the extracellular antigen binding domain of the second receptor specifically binds an allelic variant of a major histocompatibility complex (MHC) protein.

5. The immune cell of any one of claims 1-4, wherein the extracellular antigen binding domain of the second receptor specifically binds an allelic variant of an HLA-A, HLA-B, or HLA-C protein.

6. The immune cell of any one of claims 1-5, wherein the extracellular antigen binding domain of the second receptor specifically binds to HLA-A*01, HLA-A*02, HLA-A*03, HLA-A*11, HLA-B*07, or HLA-C*07.

7. The immune cell of any one of claims 1-3, wherein the non-target antigen is encoded by an HLA class I allele.

8. The immune cell of any one of claims 1-7, wherein the cancer cell expresses HLA-E.

9. The immune cell of claim 8, wherein the HLA-E*01 comprises HLA-E*01:01 or HLA-E*01:03.

10. The immune cell of any one of claims 1-9, wherein the cancer cell is a colorectal renal cancer cell, an ovarian cancer cell, a cervical cancer cell, a melanoma cancer cell, a urothelial cancer cell, a pancreatic cancer cell, a gastric cancer cell, a head and neck cancer cell, a lung cancer cell, a breast cancer cell or a lymphoma cell.

11. The immune cell of any one of claims 1-10, wherein the non-target antigen is expressed by healthy cells of a subject.

12. The immune cell of any one of claims 1-11, wherein healthy cells of the subject express both the target antigen and non-target antigen.

13. The immune cell of any one of claims 1-12, wherein the activator receptor and the inhibitory receptor together can specifically activate the immune cell in the presence of the cancer cell.

14. The immune cell of any one of claims 1-13, wherein the immune cell is a T cell, an NK cell or a macrophage.

15. The immune cell of claim 14, wherein the T cell is a CD8+CD4− T cell, or wherein the T cell is a CD8− CD4+ T cell.

16. The immune cell of any one of claims 1-15, wherein the activator receptor is a T cell receptor (TCR) or a chimeric antigen receptor (CAR).

17. The immune cell of claim 16, wherein the extracellular antigen binding domain of the activator receptor comprises an antibody fragment, a single chain Fv antibody fragment (scFv), a β chain variable domain (Vβ), or a TCR α chain variable domain and a TCR β chain variable domain.

18. The immune cell of claim 16, wherein the extracellular antigen binding domain of the activator receptor is an scFv.

19. The immune cell of claim 17 or 18, wherein the extracellular antigen binding domain of the activator receptor comprises a heavy chain variable (VH) region and a light chain variable (VL) region.

20. The immune cell of claim 19, wherein the VH region of the activator receptor comprises complement determining region (CDR) sequences of SYGVH (SEQ ID NO: 21979), VIWTGGTTNYNTALMS (SEQ ID NO: 21980), or GFSLTSY (SEQ ID NO: 4), WTGGT (SEQ ID NO: 5) and DGDSYNREAWFAY (SEQ ID NO: 6), or sequences having at most 1, 2, or 3 substitutions, deletions, or insertions relative to SEQ ID NOS: 4-6, 21979, or 21980.

21. The immune cell of claim 19 or 20, wherein the VL region of the activator receptor comprises CDRs of SASSSVSYMH (SEQ ID NO: 1), GTSNLAS (SEQ ID NO: 2) and QQRSRYPFT (SEQ ID NO: 3), having at most 1, 2, or 3 substitutions, deletions, or insertions relative to SEQ ID NOS: 1-3.

22. The immune cell of any one of claims 19-21, wherein the VH region of the activator receptor comprises a sequence of QVQLRESGPSLVKPSQTLSLTCTVSGFSLTSYGVHWVRQPPGKGLEWLGVIWTGG TTNYNTALMSRLSITKDNSKSQVFLKMNSLQTDDTAIYYCARDGDSYNREAWFA YWGQGTLVTVSA (SEQ ID NO: 8), or a sequence having at least 90%, at least 95%, at least 96%, at least 97%, at least 98%, or at least 99% identity thereto.

23. The immune cell of any one of claims 19-22, wherein the VL region of the activator receptor comprises a sequence of QIVLTQSPAVISASPGEKVILTCSASSSVSYMHWFQQKPGTSPKLWIYGTSNLASG VPARFSGGGSGTSYSLTISRMEAEDAATYYCQQRSRYPFTFGSGTKLEIK (SEQ ID NO: 7), or a sequence having at least 90%, at least 95%, at least 96%, at least 97%, at least 98%, or at least 99% identity thereto.

24. The immune cell of any one of claims 16-23, wherein the CAR comprises a hinge sequence isolated or derived from CD8, CD28, IgG1, or IgG4, or a synthetic hinge.

25. The immune cell of any one of claims 16-24, wherein the CAR comprises a transmembrane domain isolated or derived from CD8 or CD28.

26. The immune cell of any one of claims 16-25, wherein the CAR comprises an intracellular domain isolated or derived from CD28, 4-1BB or CD3z, or a combination thereof.

27. The immune cell of any one of claims 1-26, wherein the inhibitory receptor comprises a TCR or CAR.

28. The immune cell of any one of claims 1-27, wherein the extracellular antigen binding domain of the inhibitory receptor comprises an antibody fragment, a single chain Fv antibody fragment (scFv), a β chain variable domain (Vβ), or a TCR α chain variable domain and a TCR β chain variable domain.

29. The immune cell of any one of claims 1-28, wherein the extracellular antigen binding domain of the inhibitory receptor comprises a heavy chain variable (VH) region and a light chain variable (VL) region.

30. The immune cell of claim 29 wherein the VH and VL regions of the inhibitory receptor comprise CDRs selected from the group of CDR sequences disclosed in Table 5, or CDR sequences having at most 1, 2, or 3 substitutions, deletions, or insertions relative to thereto.

31. The immune cell of any one of claims 1-30, wherein the inhibitory receptor comprises an extracellular antigen binding domain selected from the group of sequences disclosed in Tables 3 and 4, or a sequence having at least 85%, at least 90%, at least 95%, at least 97%, or at least 99% identity thereto.

32. The immune cell of any one of claims 1-31, wherein the inhibitory receptor comprises a LILRB1 intracellular domain or a functional variant thereof.

33. The immune cell of any one of claims 1-32, wherein the inhibitory receptor comprises LILRB1 hinge and transmembrane domains, or functional variants thereof.

34. The immune cell of claim 32 or 33, wherein the VH region of the inhibitory receptor comprises (a) a heavy chain CDR1 selected from the group consisting of SGGYYWS (SEQ ID NO: 10), TSGVGVG (SEQ ID NO: 11), SYAMH (SEQ ID NO: 12), SYDMH (SEQ ID NO: 13), SYWMH (SEQ ID NO: 14), and SYHIH (SEQ ID NO: 21986); (b) a HC CDR2 sequence selected from the group consisting of YIYYSGSTYYNPSLKS (SEQ ID NO: 15), LIYWNDDKRYSPSLKS (SEQ ID NO: 16), WINAGNGNTKYSQKFQG (SEQ ID NO: 17), AIGTAGDTYYPGSVKG (SEQ ID NO: 18), RINSDGSSTSYADSVKG (SEQ ID NO: 19), and WIYPGNVNTEYNEKFKG (SEQ ID NO: 21987); and (c) a HC CDR3 sequence selected from the group consisting of HYYYYSMDV (SEQ ID NO: 20), HYYYYYLDV (SEQ ID NO: 21), HYYYYMDV (SEQ ID NO: 22), HYYYYYMDV (SEQ ID NO: 23), KTTSFYFDY (SEQ ID NO: 24), RHMRLSCFDY (SEQ ID NO: 25), EGNGANPDAFDI (SEQ ID NO: 26), DLPGSYWYFDL (SEQ ID NO: 27), GVLLYNWFDP (SEQ ID NO: 28), and EEITYAMDY (SEQ ID NO: 101).

35. The immune cell of any one of claims 32-34, wherein the VL region comprises a LC CDR1 comprising a sequence of RSSQSIVHSNGNTYLE (SEQ ID NO: 87) or RASQSISSYLN (SEQ ID NO: 29), a LC CDR2 comprising a sequence of KVSNRFS (SEQ ID NO: 21985) or AASSLQS (SEQ ID NO: 30) and a LC CDR3 comprising a sequence of FQGSHVPRT (SEQ ID NO: 92) or QQSYSTPLT (SEQ ID NO: 31).

36. The immune cell of any one of claims 32-35, wherein the scFv of the inhibitory receptor comprises a sequence having at least 85%, at least 90%, at least 95%, at least 97%, at least 99% identity or is identical to a sequence selected from the group consisting of SEQ ID NOS: 52-60.

37. The immune cell of any one of claims 1-36, wherein the inhibitory receptor comprises a sequence having at least 85%, at least 90%, at least 95%, at least 97%, at least 99% identity or is identical to
(SEQ ID NO: 254)
YGSQSSKPYLLTHPSDPLELVVSGPSGGPSSPTTGPTSTSGPEDQPLTP
TGSDPQSGLGRHLGVVIGILVAVILLLLLLLLLFLILRHRRQGKHWTST
QRKADFQHPAGAVGPEPTDRGLQWRSSPAADAQEENLYAAVKHTQPEDG
VEMDTRSPHDEDPQAVTYAEVKHSRPRREMASPPSPLSGEFLDTKDRQA
EEDRQMDTEAAASEAPQDVTYAQLHSLTLRREATEPPPSQEGPSPAVPS
IYATLAIH.

38. The immune cell of any one of claims 1-37, wherein expression and/or function of a MHC Class I gene has been reduced or eliminated.

39. The immune cell of claim 38, wherein the MHC Class I gene is beta-2-microglobulin (B2M).

40. The immune cell of claim 39, further comprising a polynucleotide comprising an interfering RNA, the interfering RNA comprising a sequence complementary to a sequence of a B2M mRNA.

41. The immune cell of claim 39, comprising one or more modifications to a sequence encoding B2M, wherein the one or more modifications reduce the expression and/or eliminate the function of B2M.

42. The immune cell of claim 41, wherein the one or more modifications are introduced with a nucleic acid guided endonuclease in a complex with at least one guide nucleic acid (gNA) that specifically targets a sequence of the endogenous gene encoding B2M.

43. The immune cell of claim 38, wherein the MHC Class I gene is HLA-A*02.

44. The immune cell of claim 43, further comprising a polynucleotide comprising an interfering RNA, comprising a sequence complementary to a sequence of an HLA-A*02 mRNA.

45. The immune cell of claim 43, comprising one or more modifications to a sequence of an endogenous gene encoding HLA-A*02, wherein the one or modifications reduce the expression and/or eliminate the function of HLA-A*02.

46. The immune cell of claim 45, wherein the one or more modifications are introduced with a nucleic acid guided endonuclease in a complex with at least one guide nucleic acid (gNA) that specifically targets a sequence of the endogenous gene encoding HLA-A*02.

47. The immune cell of any one of claims 1-46, wherein the immune cell is autologous.

48. The immune cell of any one of claims 1-46, wherein the immune cell is allogeneic.

49. A pharmaceutical composition, comprising a therapeutically effective amount of the immune cells of any one of claims 1-48.

50. The pharmaceutical composition of claim 49, further comprising a pharmaceutically acceptable carrier, diluent or excipient.

51. The pharmaceutical composition of claim 49 or 50, for use as a medicament in the treatment of cancer.

52. A polynucleotide or polynucleotide system, comprising one or more polynucleotides comprising polynucleotide sequences encoding:
a. an activator receptor comprising an extracellular antigen binding domain specific to an human leukocyte antigen E (HLA-E) antigen expressed by a cancer cell; and
b. an inhibitory receptor comprising an extracellular antigen binding domain specific to a non-target antigen that is not expressed by the cancer cell.

53. A vector, comprising the polynucleotide or polynucleotide system of claim 52.

54. A method of selectively killing and/or inhibiting the proliferation of an HLA-E positive cell exhibiting loss-of-heterozygosity or loss of expression of a non-target antigen, comprising contracting the HLA-E positive cells with the immune cells of any one of claims 1-48.

55. A method of treating an HLA-E positive cancer in a subject, comprising administering to the subject the immune cells of any one of claims 1-48.

56. A method of making an immune cell therapy, comprising transforming immune cells with the polynucleotide or polynucleotide system of claim 52 or the vector of claim 53.

57. The method of claim 56, wherein the method comprises, after the transforming step, expanding the immune cells ex vivo without stimulatory factors.

58. A kit comprising the immune cell of any one of claims 1-48 or the pharmaceutical composition of any one of claims 49-51.

59. The kit of claim 58, further comprising instructions for use.

60. A method of making an immune cell therapy, comprising:
a. transforming immune cells with the polynucleotide or polynucleotide system of claim 52 or the vector of claim 53; and
b. reducing or eliminating expression and/or function of beta-2-microglobulin (B2M).
